(19) United States
(12) Patent Application Publication
(10) Pub. No.: US 2009/0275021 A1
(43) Pub. Date: Nov. 5, 2009

(54) NOVEL NUCLEOTIDE AND AMINO ACID SEQUENCES, AND ASSAYS AND METHODS OF USE THEREOF FOR DIAGNOSIS

(76) Inventors: Osnat Sella-Tavor, Kfar-Kish (IL); Sarah Pollock, Tel-Aviv (IL); Lily Bazak, Givatayim (IL); Elena Tsypkin, Tel-Aviv (IL); Shira Wallach, Hod-hasharon (IL); Eve Montia, Rehovot (IL); Shirley Sameah-Greenwald, Kfar Saba (IL)

Correspondence Address: FENNEMORE CRAIG, 3003 NORTH CENTRAL AVENUE, SUITE 2600, PHOENIX, AZ, 85012, US

(21) Appl. No.: 12/095,110

(22) PCT Filed: Nov. 27, 2006
(86) PCT No.: PCT/IL06/01365

§ 371 Date: Dec. 9, 2008

Related U.S. Application Data
(60) Provisional application No. 60/751,378, filed on Dec. 19, 2005.

(30) Foreign Application Priority Data
Nov. 27, 2005 (IL) 172204
Jan. 16, 2006 (IL) 173173
Aug. 1, 2006 (IL) 177215
Dec. 8, 2006 (IL) 172460

(51) Int. Cl. C12Q 1/68; C12N 15/11; C07K 14/00; C07K 16/18; C12N 15/00; C07H 21/04; C12N 5/06; C12P 21/02; G01N 33/53

(52) U.S. Cl. 435/6; 536/23.5; 530/350; 530/387.9; 435/320.1; 435/325; 435/69.1; 435/7.23; 536/24.33

(57) ABSTRACT

Novel splice variants, amino acid sequences and nucleotide sequences thereof, and methods of using same.

### FIELD OF THE INVENTION

The present invention is related to novel nucleotide acid sequences, and assays and methods of use thereof.

### BACKGROUND OF THE INVENTION

Diagnostic markers are important for early diagnosis of many diseases, as well as predicting response to treatment, monitoring treatment and determining prognosis of such diseases.

Nucleic Acid Testing (NAT) is a subset of molecular diagnostic markers, based on testing for the presence of a nucleic acid sequence in a sample, associated with a certain condition (most often a clinical pathology). The sample could be a body fluid, a tissue sample, a body secretion or any other sample obtained from a patient which could contain the targeted nucleic acids.

Traditionally, NAT diagnosis has been used for the diagnosis of infectious diseases. Particularly, it has been used for the diagnosis of HIV, Hepatitis C Virus (HCV), Hepatitis B Virus (HBV), Chlamydia trachomatis, Neisseria gonorrhoeae and Mycobacteria tuberculosis. In recent years NAT diagnosis has expanded to noninfectious diseases, for example, for the diagnosis of prostate cancer based on DD3 (PCA3). DD3 (PCA3) is a very prostate cancer-specific gene. It has shown a great diagnostic value for prostate cancer by measuring quantitavely the DD3 (PCA3) transcript in urine sediments obtained after prostatic massage. DD3 (PCA3) is a non-coding transcript, therefore diagnosis in the protein level is not possible. More NAT markers for more cancers in addition to prostate cancer are currently pursued.

NAT diagnostic markers have at least four advantages on protein based diagnostic modalities:

1) They are likely to be more sensitive and specific (as has been shown for diagnostic kits for HIV and HCV). This finding could be related to at least two things:
a. The test analyte could be amplified (e.g. with PCR)
b. The detection method is sequence specific rather than epitope specific
2) They allow diagnosis even if a differentially expressed transcript is non-coding (as in the case of DD3(PCA3))
3) The research tools for the discovery of novel NAT markers are much more advanced and robust than for protein markers (e.g. advanced DNA chip technology compared with protein chip technology)
4) NAT analytes are sometimes found in body secretions and/or body fluids and therefore could replace the need for a tissue biopsy when a serum marker is not available.

However, NAT markers suffer from a few disadvantages including:

1) The analyte itself is quite an unstable molecule (certainly when compared with a protein).
2) The analyte itself is by nature not physiologically secreted, therefore it is not always easily found in samples.

NAT markers development for noninfectious diseases was not pursued for a long time, which was mostly a result of expensive and not fully developed detection methods on one hand and intellectual property barriers on the other. With the advance in technology and expiration of key patents in the field, the industry is investing more and more resources in that direction and it seems that NAT based tests are going to be much more prevalent for noninfectious diseases in the future.

Another example of diagnostic markers which are used for diagnosis of many different diseases are serum markers. Such serum markers typically encompass secreted proteins and/or peptides; however, some serum markers may be released to the blood upon tissue lysis, such as from myocardial infarction (for example Troponin-I). Serum markers can also be used as risk factors for disease (for example base-line levels of CRP, as a predictor of cardiovascular disease), to monitor disease activity and progression (for example, determination of CRP levels to monitor acute phase inflammatory response) and to predict and monitor drug response (for example, as shredded fragments of the protein Erb-B2).

Immunohistochemistry (IHC) is the study of distribution of an antigen of choice in a sample based on specific antibody-antigen binding, typically on tissue slices. The antibody features a label which can be detected, for example as a stain which is detectable under a microscope. The tissue slices are prepared by being fixed. IHC is therefore particularly suitable for antibody-antigen reactions that are not disturbed or destroyed by the process of fixing the tissue slices.

IHC permits determining the localization of binding, and hence mapping of the presence of the antigen within the tissue and even within different compartments in the cell. Such mapping can provide useful diagnostic information, including:

1) the histological type of the tissue sample
2) the presence of specific cell types within the sample
3) information on the physiological and/or pathological state of cells (e.g. which phase of the cell-cycle they are in)
4) the presence of disease related changes within the sample
5) differentiation between different specific disease subtypes where it is already known the tissue is of disease state (for example, the differentiation between different tumor types when it is already known the sample was taken from cancerous tissue).
IHC information is valuable for more than diagnosis. It can also be used to determine prognosis and therapy treatment (as in the case of HER-2 in breast cancer) and monitor disease.

IHC protein markers could be from any cellular location. Most often these markers are membrane proteins but secreted proteins or intracellular proteins (including intranuclear) can be used as an IHC marker too.

IHC has at least two major disadvantages. It is performed on tissue samples and therefore a tissue sample has to be collected from the patient, which most often requires invasive procedures like biopsy associated with pain, discomfort, hospitalization and risk of infection. In addition, the interpretation of the result is observer dependant and therefore subjective. There is no measured value but rather only an estimation (on a scale of 1-4) of how prevalent the antigen on target is.

### SUMMARY OF THE INVENTION

The present invention provides, in different embodiments, many novel nucleic acid and amino acid sequences, which may optionally be used as diagnostic markers.

In some embodiments the present invention overcomes deficiencies of the background art by providing novel variants that are suitable for use with NAT and/or nucleic acid hybridization methods and assays, which may optionally be used as diagnostic markers. Collectively, methods and assays that are suitable for detecting a nucleic acid sequence (oligonucleotides) are referred to herein as “oligonucleotide detection technologies”, including but not limited to NAT and hybridization technologies. The markers of the present invention may optionally be used with any such oligonucleotide detection technology.

In some embodiments, the present invention provides a number of different variants of known proteins, which are expressed in serum and may optionally be used as diagnostic markers, which in some embodiments, are serum markers, or in other embodiments, are IHC markers. The present invention therefore overcomes the many deficiencies of the background art with regard to the need to obtain tissue samples and subjective interpretations of results. In one embodiment, tissue specific markers are identifible in serum or plasma. In some embodiments, a simple blood test can provide qualitative and/or quantitative indicators for expression of a desired marker, for example, serving as an indicator for various diseases and/or pathological conditions. The markers presented in the present invention can also potentially be used for in-vivo imaging applications.

The diseases for which such variants may be useful as diagnostic markers are described in greater detail below. The variants themselves are described by “cluster” or by gene, as these variants are splice variants of known proteins. In some embodiments, the term “marker-detectable disease” refers to a disease that may be detected by a particular marker, with regard to the description of such diseases below. In some embodiments, the markers of the present invention, alone or in combination, show a high degree of differential detection between disease and non-disease states.

The present invention therefore also relates to diagnostic assays for disease detection optionally and preferably in a sample taken from a subject (patient), which is more preferably some type of blood sample or body secretion sample. The assays are optionally NAT (nucleic acid amplification technology)-based assays, such as PCR for example (or variations thereof such as real-time PCR for example). The assays may also optionally encompass nucleic acid hybridization assays. The assays may optionally be qualitative or quantitative.

The present invention relates, in some embodiments, to diagnostic assays for disease detection, which in some embodiments, utilizes a biological sample taken from a subject (patient), which for example may comprise a body fluid or secretion including but not limited to seminal plasma, blood, serum, urine, prostatic fluid, seminal fluid, semen, the external secretions of the skin, respiratory, intestinal, and genitourinary tracts, tears, cerebrospinal fluid, sputum, saliva, milk, peritoneal fluid, pleural fluid, cyst fluid, secretions of the breast ductal system (and/or lavage thereof), broncho alveolar lavage, lavage of the reproductive system, lavage of any other part of the body or system in the body, samples of any organ including but not limited to lung, colon, ovarian and/or breast tissue, stool or a tissue sample, or any combination thereof. In some embodiments, the term encompasses samples of in vivo cell culture constituents. The sample can optionally be diluted with a suitable eluant before contacting the sample to an antibody and/or performing any other diagnostic assay.

In some embodiments of the present invention, nucleic acids, or polypeptides, having a sequence as described herein, or homologues thereof.

In some embodiments, the terms “homology”, “homologue” or “homologous”, in any instance, indicate that the sequence referred to, whether an amino acid sequence, or a nucleic acid sequence, exhibits, in one embodiment at least 70% correspondence with the indicated sequence. In another embodiment, the amino acid sequence or nucleic acid sequence exhibits at least 72% correspondence with the indicated sequence. In another embodiment, the amino acid sequence or nucleic acid sequence exhibits at least 75% correspondence with the indicated sequence. In another embodiment, the amino acid sequence or nucleic acid sequence exhibits at least 77% correspondence with the indicated sequence. In another embodiment, the amino acid sequence or nucleic acid sequence exhibits at least 80% correspondence with the indicated sequence. In another embodiment, the amino acid sequence or nucleic acid sequence exhibits at least 82% correspondence with the indicated sequence. In another embodiment, the amino acid sequence or nucleic acid sequence exhibits at least 85% correspondence with the indicated sequence. In another embodiment, the amino acid sequence or nucleic acid sequence exhibits at least 87% correspondence with the indicated sequence. In another embodiment, the amino acid sequence or nucleic acid sequence exhibits at least 90% correspondence with the indicated sequence. In another embodiment, the amino acid sequence or nucleic acid sequence exhibits at least 92% correspondence with the indicated sequence. In another embodiment, the amino acid sequence or nucleic acid sequence exhibits at least 95% or more correspondence with the indicated sequence. In another embodiment, the amino acid sequence or nucleic acid sequence exhibits 95%-100% correspondence to the indicated sequence. Similarly, in some embodiments, the reference to a correspondence to a particular sequence includes both direct correspondence, as well as homology to that sequence as herein defined.

In some embodiments, this invention provides an isolated polynucleotide comprising a nucleic acid having a sequence corresponding to, or homologous to that set forth in SEQ ID NOs: 1-3, 87-92, 155-158, 187-193, 334, 346, 421, 440-449.

In some embodiments, this invention provides an isolated polynucleotide segment comprising a nucleic acid having a sequence corresponding to, or homologous to that set forth in SEQ ID NOs: 4-28, 53-72, 93-132, 159-172, 194-204, 347-349, 450-461, 481-484.

In some embodiments, this invention provides an isolated protein or polypeptide having an amino acid sequence corresponding to, or homologous to that set forth in SEQ ID NOs: 33-36, 140, 142-144, 183-186, 213-214, 217, 355, 469-471, 474-475.

In some embodiments, the proteins or polypeptides of this invention comprise chimeric protein or polypeptides.

In some embodiments, the terms “chimeric protein or polypeptide”, or “chimera” refers to an assembly or a string of amino acids in a particular sequence, or nucleotides encoding the same, respectively, which does not correspond to the sequence of the known (wild type) polypeptide or protein, or nucleic acid, respectively. In some embodiments, the variants of this invention are derived by the by the assembly or stringing of amino acids or polynucleotides encoding the same, from two exons, or an exon and an intron, or fragments thereof, or segments having sequences with the indicated homology.

In some embodiments, the isolated chimeric proteins or polypeptides of the invention may comprise an amino acid sequence corresponding to or homologous to that set forth in SEQ ID NO: 33-36, 139-144, 183-186, 213-217, 355, 469-476.

In some embodiments, such isolated chimeric proteins or polypeptides may comprise an amino acid sequence corresponding to or homologous to that set forth in AA383349_P4 (SEQ ID NO: 33).

In some embodiments, the isolated chimeric proteins or polypeptides of the invention may comprise an amino acid sequence corresponding to or homologous to that as set forth in AA383349_P4 (SEQ ID NO: 33), comprising a amino acid sequence being at least about 90% homologous to amino acids 151-440 of Q96EZ7_HUMAN (SEQ ID NO: 30), which also corresponds to amino acids 1-290 of AA383349_P4 (SEQ ID NO: 33).

In some embodiments, the isolated chimeric proteins or polypeptides of the invention may comprise an amino acid sequence corresponding to or homologous to that as set forth in AA383349_P4 (SEQ ID NO: 33), comprising a first amino acid sequence being at least about 90% homologous to amino acids 1-17 of NP_{—}005158 (SEQ ID NO: 31), which also corresponds to amino acids 1-17 of AA383349_P4 (SEQ ID NO: 33), a second amino acid sequence being at least about 70%, optionally at least about 80%, preferably at least about 85%, more preferably at least about 90% and most preferably at least about 95% homologous to a polypeptide having the sequence VEILQDPSPPPLCLPTTPGTPDSSDPSHLLGPQSCWSSQ (SEQ ID NO: 494) corresponding to amino acids 18-56 of AA383349_P4 (SEQ ID NO: 33), and a third amino acid sequence being at least about 90% homologous to amino acids 18-251 of NP_{—}005158 (SEQ ID NO: 31), which also corresponds to amino acids 57-290 of AA383349_P4 (SEQ ID NO: 33), wherein said first amino acid sequence, second amino acid sequence and third amino acid sequence are contiguous and in a sequential order.

In some embodiments, this invention provides an isolated polypeptide encoding for an edge portion of AA383349_P4 (SEQ ID NO: 33), comprising an amino acid sequence being at least about 70%, optionally at least about 80%, preferably at least about 85%, more preferably at least about 90% and most preferably at least about 95% homologous to the sequence VEILQDPSPPPLCLPTTPGTPDSSDPSHLLGPQSCWSSQ (SEQ ID NO: 494) of AA383349_P4 (SEQ ID NO: 33).

In some embodiments, such isolated chimeric proteins or polypeptides may comprise an amino acid sequence corresponding to or homologous to that set forth in AA383349_P5 (SEQ ID NO: 34).

In some embodiments, the isolated chimeric proteins or polypeptides of the invention may comprise an amino acid sequence corresponding to or homologous to that as set forth in AA383349_P5 (SEQ ID NO: 34), comprising a amino acid sequence being at least about 90% homologous to amino acids 1-457 of Q6DKJ7_HUMAN (SEQ ID NO:429), which also corresponds to amino acids 1-457 of AA383349_P5 (SEQ ID NO: 34).

In some embodiments, the isolated chimeric proteins or polypeptides of the invention may comprise an amino acid sequence corresponding to or homologous to that as set forth in AA383349_P5 (SEQ ID NO: 34), comprising a first amino acid sequence being at least about 70%, optionally at least 80%, preferably at least 85%, more preferably at least 90% and most preferably at least 95%, homologous to a polypeptide having the sequence MLNRVRSAVAHLVSSGG (SEQ ID NO: 495) corresponding to amino acids 1-17 of AA383349_P5 (SEQ ID NO: 34), and a second amino acid sequence being at least about 90% homologous to amino acids 1-440 of Q96EZ7_HUMAN (SEQ ID NO: 30), which also corresponds to amino acids 18-457 of AA383349_P5 (SEQ ID NO: 34), wherein said first amino acid sequence and second amino acid sequence are contiguous and in a sequential order.

In some embodiments, this invention provides an isolated polypeptide encoding for a head of AA383349_P5 (SEQ ID NO: 34), comprising a polypeptide being at least 70%, optionally at least about 80%, preferably at least about 85%, more preferably at least about 90% and most preferably at least about 95% homologous to the sequence MLNRVRSAVAHLVSSGG (SEQ ID NO: 495) of AA383349_P5 (SEQ ID NO: 34).

In some embodiments, the isolated chimeric proteins or polypeptides of the invention may comprise an amino acid sequence corresponding to or homologous to that as set forth in AA383349_P5 (SEQ ID NO: 34), comprising a first amino acid sequence being at least about 70%, optionally at least 80%, preferably at least 85%, more preferably at least 90% and most preferably at least 95%, homologous to a polypeptide having the sequence MLNRVRSAVAHLVSSGGAPPPRPKSPDLPNAASAPPAAAPEAPRSPPAKAGSGSATPAKAVEARASFSRPT FLQLSPGGLRRADDHAGRAVQSPPDTGRRLPWSTGYAEVINAGKSRHNEDQACCEVVYVEGRRSVTGVP REPSRGQGLCFYYWGLFDGHAGGGAAE (SEQ ID NO: 496) corresponding to amino acids 1-167 of AA383349_P5 (SEQ ID NO: 34), a second amino acid sequence being at least about 90% homologous to amino acids 1-17 of NP_{—}005158 (SEQ ID NO: 31), which also corresponds to amino acids 168-184 of AA383349_P5 (SEQ ID NO: 34), a third amino acid sequence being at least about 70%, optionally at least 80%, preferably at least 85%, more preferably at least 90% and most preferably at least 95% homologous to a polypeptide having the sequence VEILQDPSPPPLCLPTTPGTPDSSDPSHLLGPQSCWSSQ (SEQ ID NO: 494) corresponding to amino acids 185-223 of AA383349_P5 (SEQ ID NO: 34), and a fourth amino acid sequence being at least about 90% homologous to amino acids 18-251 of NP_{—}005158 (SEQ ID NO: 31), which also corresponds to amino acids 224-457 of AA383349_P5 (SEQ ID NO: 34), wherein said first amino acid sequence, second amino acid sequence, third amino acid sequence and fourth amino acid sequence are contiguous and in a sequential order.

In some embodiments, this invention provides an isolated polypeptide encoding for a head of AA383349_P5 (SEQ ID NO: 34), comprising a polypeptide being at least 70%, optionally at least about 80%, preferably at least about 85%, more preferably at least about 90% and most preferably at least about 95% homologous to the sequence

(SEQ ID NO: 496) of AA383349_P5 (SEQ ID NO: 34)

MLNRVRSAVAHLVSSGGAPPPRPKSPDLPNAASAPPAAAPEAPRSPPAKA
GSGSATPAKAVEARASFSRPTFLQLSPGGLRRADDHAGRAVQSPPDTGRR
LPWSTGYAEVINAGKSRHNEDQACCEVVYVEGRRSVTGVPREPSRGQGLC
FYYWGLFDGHAGGGAAE.

In some embodiments, this invention provides an isolated polypeptide encoding for an edge portion of AA383349_P5 (SEQ ID NO: 34), comprising an amino acid sequence being at least about 70%, optionally at least about 80%, preferably at least about 85%, more preferably at least about 90% and most preferably at least about 95% homologous to the sequence VEILQDPSPPPLCLPTTPGTPDSSDPSHLLGPQSCWSSQ (SEQ ID NO: 494) of AA383349_P5 (SEQ ID NO: 34).

In some embodiments, such isolated chimeric proteins or polypeptides may comprise an amino acid sequence corresponding to or homologous to that set forth in AA383349_P12 (SEQ ID NO: 36).

In some embodiments, the isolated chimeric proteins or polypeptides of the invention may comprise an amino acid sequence corresponding to or homologous to that as set forth in AA383349_P12 (SEQ ID NO: 36), comprising a first amino acid sequence being at least about 90% homologous to amino acids 1-349 of Q6DKJ7_HUMAN (SEQ ID NO:429), which also corresponds to amino acids 1-349 of AA383349_P12 (SEQ ID NO: 36), and a second amino acid sequence LG corresponding to amino acids 350-351 of AA383349_P12 (SEQ ID NO: 36), wherein said first amino acid sequence and second amino acid sequence are contiguous and in a sequential order.

In some embodiments, the isolated chimeric proteins or polypeptides of the invention may comprise an amino acid sequence corresponding to or homologous to that as set forth in AA383349_P12 (SEQ ID NO: 36), comprising a first amino acid sequence being at least about 70%, optionally at least 80%, preferably at least 85%, more preferably at least 90% and most preferably at least 95%, homologous to a polypeptide having the sequence MLNRVRSAVAHLVSSGG (SEQ ID NO: 495) corresponding to amino acids 1-17 of AA383349_P12 (SEQ ID NO: 36), a second amino acid sequence being at least about 90% homologous to amino acids 1-332 of Q96EZ7_HUMAN (SEQ ID NO: 30), which also corresponds to amino acids 18-349 of AA383349_P12 (SEQ ID NO: 36), and a third amino acid sequence LG corresponding to amino acids 350-351 of AA383349_P12 (SEQ ID NO: 36), wherein said first amino acid sequence, second amino acid sequence and third amino acid sequence are contiguous and in a sequential order.

In some embodiments, this invention provides an isolated polypeptide encoding for a head of AA383349_P12 (SEQ ID NO: 36), comprising a polypeptide being at least 70%, optionally at least about 80%, preferably at least about 85%, more preferably at least about 90% and most preferably at least about 95% homologous to the sequence MLNRVRSAVAHLVSSGG (SEQ ID NO: 495) of AA383349_P12 (SEQ ID NO: 36).

In some embodiments, the isolated chimeric proteins or polypeptides of the invention may comprise an amino acid sequence corresponding to or homologous to that as set forth in H13410_P4 (SEQ ID NO: 139), comprising a first amino acid sequence being at least about 90% homologous to amino acids 1-515 of Q6UXG2_HUMAN (SEQ ID NO: 137), which also corresponds to amino acids 1-515 of H13410_P4 (SEQ ID NO: 139), and a second amino acid sequence being at least about 70%, optionally at least 80%, preferably at least 85%, more preferably at least 90% and most preferably at least 95% homologous to a polypeptide having the sequence VRFPFFAR (SEQ ID NO: 499) corresponding to amino acids 516-523 of H13410_P4 (SEQ ID NO: 139), wherein said first amino acid sequence and second amino acid sequence are contiguous and in a sequential order.

In some embodiments, this invention provides an isolated polypeptide encoding for an edge portion of H13410_P4 (SEQ ID NO: 139), comprising an amino acid sequence being at least about 70%, optionally at least about 80%, preferably at least about 85%, more preferably at least about 90% and most preferably at least about 95% homologous to the sequence VRFPFFAR (SEQ ID NO: 499) of H13410_P4 (SEQ ID NO: 139).

In some embodiments, such isolated chimeric proteins or polypeptides may comprise an amino acid sequence corresponding to or homologous to that set forth in H13410_P8 (SEQ ID NO: 140).

In some embodiments, the isolated chimeric proteins or polypeptides of the invention may comprise an amino acid sequence corresponding to or homologous to that as set forth in H13410_P8 (SEQ ID NO: 140), comprising a first amino acid sequence being at least about 90% homologous to amino acids 1-876 of Q6UXG2_HUMAN (SEQ ID NO: 137), which also corresponds to amino acids 1-876 of H13410_P8 (SEQ ID NO: 140), and a second amino acid sequence being at least about 70%, optionally at least 80%, preferably at least 85%, more preferably at least 90% and most preferably at least 95% homologous to a polypeptide having the sequence VGFPLIGHFHERREWEI (SEQ ID NO: 500) corresponding to amino acids 877-893 of H13410_P8 (SEQ ID NO: 140), wherein said first amino acid sequence and second amino acid sequence are contiguous and in a sequential order.

In some embodiments, this invention provides an isolated polypeptide encoding for an edge portion of H13410_P8 (SEQ ID NO: 140), comprising an amino acid sequence being at least about 70%, optionally at least about 80%, preferably at least about 85%, more preferably at least about 90% and most preferably at least about 95% homologous to the sequence VGFPLIGHFHERREWEI (SEQ ID NO: 500) of H13410_P8 (SEQ ID NO: 140).

In some embodiments, the isolated chimeric proteins or polypeptides of the invention may comprise an amino acid sequence corresponding to or homologous to that as set forth in H13410_P8 (SEQ ID NO: 140), comprising a first amino acid sequence being at least about 90% homologous to amino acids 1-622 of NP_{—}065826 (SEQ ID NO:344), which also corresponds to amino acids 1-622 of H13410_P8 (SEQ ID NO: 140), a bridging amino acid P corresponding to amino acid 623 of H13410_P8 (SEQ ID NO: 140), a second amino acid sequence being at least about 90% homologous to amino acids 624-876 of NP_{—}065826 (SEQ ID NO:344), which also corresponds to amino acids 624-876 of H13410_P8 (SEQ ID NO: 140), and a third amino acid sequence being at least about 70%, optionally at least 80%, preferably at least 85%, more preferably at least 90% and most preferably at least 95% homologous to a polypeptide having the sequence VGFPLIGHFHERREWEI (SEQ ID NO: 500) corresponding to amino acids 877-893 of H13410_P8 (SEQ ID NO: 140), wherein said first amino acid sequence, bridging amino acid, second amino acid sequence and third amino acid sequence are contiguous and in a sequential order.

In some embodiments, the isolated chimeric proteins or polypeptides of the invention may comprise an amino acid sequence corresponding to or homologous to that as set forth in H13410_P8 (SEQ ID NO: 140), comprising a first amino acid sequence being at least about 70%, optionally at least 80%, preferably at least 85%, more preferably at least 90% and most preferably at least 95%, homologous to a polypeptide having the sequence MAEPGHSHHLSARVRGRTERRIPRLWRLLLWAGTAFQVTQGTGPELHACKESEYHYEYTACDSTGSRWR VAVPHTPGLCTSLPDPVKGTEC SFSCNAGEFLDMKDQSCKPCAEGRYSLGTGIRFDEWDELPHGFASL SAN MELDDSAAESTGNCTSSKWVPRGDYIASNTDECTATLMYAVNLKQSGTVNFEYYYPDSSIIFEFFVQNDQ CQPNADDSRWMKTTEKGWEFHSVELNRGNNVLYWRTTAFSVW KVPKPVLVRNIAITGVAYTSECFPCK PGTYADKQGSSFCKLCPANSYSNKGETSCHQCDPDKYSEKGSSSCNVRPACTDKDYFYTHTACDANGET QL (SEQ ID NO: 501) corresponding to amino acids 1-350 of H13410_P8 (SEQ ID NO: 140), a second amino acid sequence being at least about 90% homologous to amino acids 1-526 of Q8N1G3_HUMAN (SEQ ID NO: 136), which also corresponds to amino acids 351-876 of H13410_P8 (SEQ ID NO: 140), and a third amino acid sequence being at least about 70%, optionally at least 80%, preferably at least 85%, more preferably at least 90% and most preferably at least 95% homologous to a polypeptide having the sequence VGFPLIGHFHERREWEI (SEQ ID NO: 500) corresponding to amino acids 877-893 of H13410_P8 (SEQ ID NO: 140), wherein said first amino acid sequence, second amino acid sequence and third amino acid sequence are contiguous and in a sequential order.

In some embodiments, this invention provides an isolated polypeptide encoding for a head of H13410_P8 (SEQ ID NO: 140), comprising a polypeptide being at least 70%, optionally at least about 80%, preferably at least about 85%, more preferably at least about 90% and most preferably at least about 95% homologous to the sequence

(SEQ ID NO: 501)

MAEPGHSHHLSARVRGRTERRIPRLWRLLLWAGTAFQVTQGTGPELHACK
ESEYHYEYTACDSTGSRWRVAVPHTPGLCTSLPDPVKGTECSFSCNAGEF
LDMKDQSCKPCAEGRYSLGTGIRFDEWDELPHGFASLSANMELDDSAAES
TGNCTSSKWVPRGDYIASNTDECTATLMYAVNLKQSGTVNFEYYYPDSSI
IFEFFVQNDQCQPNADDSRWMKTTEKGWEFHSVELNRGNNVLYWRTTAFS
VWTKVPKPVLVRNIAITGVAYTSECFPCKPGTYADKQGSSFCKLCPANSY
SNKGETSCHQCDPDKYSEKGSSSGNVRPACTDKDYFYTHTACDANGETQL
of

(SEQ ID NO: 140)

H13410_P8.

In some embodiments, the isolated chimeric proteins or polypeptides of the invention may comprise an amino acid sequence corresponding to or homologous to that as set forth in H13410_P8 (SEQ ID NO: 140), comprising a first amino acid sequence being at least about 70%, optionally at least 80%, preferably at least 85%, more preferably at least 90% and most preferably at least 95%, homologous to a polypeptide having the sequence MAEPGHSHHLSARVRGRTERRIPRLWRLLLWAGTAFQVTQGTGPELHACKESEYHYEYTACDSTGSRWR VAVPHTPGLCTSLPDPVKGTECSFSCNAGEFLDMKDQSCKPCAEGRYSLGTGIRFDEWDELPHGFASLSAN MELDDSAAESTGNCTSSKWVPRGDYIASNTDECTATLMYAVNLKQSGTVNFEYYYPDSSIIFEFFVQNDQ CQPNADDSRWMKTTEKGWEFHSVELNRGNNVLYWRTTAFSVWTKVPKPVLVRNIAITGVAYTSECFPCK PGTYADKQGSSFCKLCPANSYSNKGETSCHQCDPDKYSEKGSSSCNVRPACTDKDYFYTHTACDANGET QL (SEQ ID NO: 501) corresponding to amino acids 1-350 of H13410_P8 (SEQ ID NO: 140), a second amino acid sequence being at least about 90% homologous to amino acids 1-272 of Q5T5C9_HUMAN (SEQ ID NO: 138), which also corresponds to amino acids 351-622 of H13410_P8 (SEQ ID NO: 140), a bridging amino acid P corresponding to amino acid 623 of H13410_P8 (SEQ ID NO: 140), a third amino acid sequence being at least about 90% homologous to amino acids 274-526 of Q5T5C9_HUMAN (SEQ ID NO: 138), which also corresponds to amino acids 624-876 of H13410_P8 (SEQ ID NO: 140), and a fourth amino acid sequence being at least about 70%, optionally at least 80%, preferably at least 85%, more preferably at least 90% and most preferably at least 95% homologous to a polypeptide having the sequence VGFPLIGHFHERREWEI (SEQ ID NO: 500) corresponding to amino acids 877-893 of H13410_P8 (SEQ ID NO: 140), wherein said first amino acid sequence, second amino acid sequence, bridging amino acid, third amino acid sequence and fourth amino acid sequence are contiguous and in a sequential order.

In some embodiments, the isolated chimeric proteins or polypeptides of the invention may comprise an amino acid sequence corresponding to or homologous to that as set forth in H13410_P8 (SEQ ID NO: 140), comprising a first amino acid sequence being at least about 70%, optionally at least 80%, preferably at least 85%, more preferably at least 90% and most preferably at least 95%, homologous to a polypeptide having the sequence MAEPGHSHHLSARVRGRTERRIPRLWRLLLWAGTAFQVTQGTGPELHACKESEYHYEYTACDSTGSRWR VAVPHTPGLCTSLPDPVKGTECSFSCNAGEFLDMKDQSCKPCAEGRYSLGTGIRFDEWDELPHGFASLSAN MELDDSAAESTGNCTSSKWVPRGDYIASNTDECTATLMYAVNLKQSGTVNFEYYYPDSSIIFEFFVQNDQ CQPNADDSRWMKTTEKGWEFHSVELNRGNNVLYWRTTAFSVWTKVPKPVLVRNIAITGVAYTSECFPCK PGTYADKQGSSFCKLCPANSYSNKGETSCHQCDPDKYSEKGSSSCNVRPACTDKDYFYTHTACDANGET QLMYKWAKPKICSEDLEGAVKLPASGVKTHCPPCNPGFFKTNNSTCQPCPYGSYSNGSDCTRCPAGTEPA VGFEYK (SEQ ID NO: 502) corresponding to amino acids 1-424 of H13410_P8 (SEQ ID NO: 140), a second amino acid sequence being at least about 90% homologous to amino acids 1-452 of Q9P2M2_HUMAN (SEQ ID NO: 135), which also corresponds to amino acids 425-876 of H13410_P8 (SEQ ID NO: 140), and a third amino acid sequence being at least about 70%, optionally at least 80%, preferably at least 85%, more preferably at least 90% and most preferably at least 95% homologous to a polypeptide having the sequence VGFPLIGHFHERREWEI (SEQ ID NO: 500) corresponding to amino acids 877-893 of H13410_P8 (SEQ ID NO: 140), wherein said first amino acid sequence, second amino acid sequence and third amino acid sequence are contiguous and in a sequential order.

In some embodiments, this invention provides an isolated polypeptide encoding for a head of H13410_P8 (SEQ ID NO: 140), comprising a polypeptide being at least 70%, optionally at least about 80%, preferably at least about 85%, more preferably at least about 90% and most preferably at least about 95% homologous to the sequence

(SEQ ID NO: 502)

MAEPGHSHHLSARVRGRTERRIPRLWRLLLWAGTAFQVTQGTGPELHACK
ESEYHYEYTACDSTGSRWRVAVPHTPGLCTSLPDPVKGTECSFSCNAGEF
LDMKDQSCKPCAEGRYSLGTGIRFDEWDELPHGFASLSANMELDDSAAES
TGNCTSSKWVPRGDYIASNTDECTATLMYAVNLKQSGTVNFEYYYPDSSI
IFEFFVQNDQCQPNADDSRWMKTTEKGWEFHSVELNRGNNVLYWRTTAFS
VWTKVPKPVLVRNIAITGVAYTSECFPCKPGTYADKQGSSFCKLCPANSY
SNKGETSCHQCDPDKYSEKGSSSCNVRPACTDKDYFYTHTACDANGETQL
MYKWAKPKICSEDLEGAVKLPASGVKTHCPPCNPGFFKTNNSTCQPCPYG
SYSNGSDCTRCPAGTEPAVGFEYK
of

(SEQ ID NO: 140)

H13410_P8.

In some embodiments, the isolated chimeric proteins or polypeptides of the invention may comprise an amino acid sequence corresponding to or homologous to that as set forth in H13410_P9 (SEQ ID NO: 141), comprising a first amino acid sequence being at least about 90% homologous to amino acids 1-936 of Q6UXG2_HUMAN (SEQ ID NO: 137), which also corresponds to amino acids 1-936 of H13410_P9 (SEQ ID NO: 141), and a second amino acid sequence being at least about 70%, optionally at least 80%, preferably at least 85%, more preferably at least 90% and most preferably at least 95% homologous to a polypeptide having the sequence YMLRYWSVGOMDRVGLLIRENI (SEQ ID NO: 503) corresponding to amino acids 937-957 of H13410_P9 (SEQ ID NO: 141), wherein said first amino acid sequence and second amino acid sequence are contiguous and in a sequential order.

In some embodiments, this invention provides an isolated polypeptide encoding for an edge portion of H13410_P9 (SEQ ID NO: 141), comprising an amino acid sequence being at least about 70%, optionally at least about 80%, preferably at least about 85%, more preferably at least about 90% and most preferably at least about 95% homologous to the sequence YMLRYWSVGMDRVGLLIRENI (SEQ ID NO: 503) of H13410_P9 (SEQ ID NO: 141).

In some embodiments, the isolated chimeric proteins or polypeptides of the invention may comprise an amino acid sequence corresponding to or homologous to that as set forth in H13410_P9 (SEQ ID NO: 141), comprising a first amino acid sequence being at least about 90% homologous to amino acids 1-622 of NP_{—}065826 (SEQ ID NO:344), which also corresponds to amino acids 1-622 of H13410_P9 (SEQ ID NO: 141), a bridging amino acid P corresponding to amino acid 623 of H13410_P9 (SEQ ID NO: 141), a second amino acid sequence being at least about 90% homologous to amino acids 624-936 of NP_{—}065826 (SEQ ID NO:344), which also corresponds to amino acids 624-936 of H13410_P9 (SEQ ID NO: 141), and a third amino acid sequence being at least about 70%, optionally at least 80%, preferably at least 85%, more preferably at least 90% and most preferably at least 95% homologous to a polypeptide having the sequence YMLRYWSVGMDRVGLLIRENI (SEQ ID NO: 503) corresponding to amino acids 937-957 of H13410_P9 (SEQ ID NO: 141), wherein said first amino acid sequence, bridging amino acid, second amino acid sequence and third amino acid sequence are contiguous and in a sequential order.

In some embodiments, the isolated chimeric proteins or polypeptides of the invention may comprise an amino acid sequence corresponding to or homologous to that as set forth in H13410_P9 (SEQ ID NO: 141), comprising a first amino acid sequence being at least about 70%, optionally at least 80%, preferably at least 85%, more preferably at least 90% and most preferably at least 95%, homologous to a polypeptide having the sequence MAEPGHSHHLSARVRGRTERRIPRLWRLLLWAGTAFQVTQGTGPELHACKESEYHYEYTACDSTGSRWR VAVPHTPGLCTSLPDPVKGTECSFSCNAGEFLDMKDQSCKPCAEGRYSLGTGIRFDEWDELPHGFASLSAN MELDDSAAESTGNCTSSKWVPRGDYIASNTDECTATLMYAVNLKQSGTVNFEYYYPDSSIIFEFFVQNDQ CQPNADDSRWMKTTEKGWEFHSVELNRGNNVLYWRTTAFSVWTKVPKPVLVRNIAITGVAYTSECFPCK PGTYADKQGSSFCKLCPANSYSNKGETSCHQCDPDKYSEKGSSSCNVRPACTDKDYFYTHTACDANGET QL (SEQ ID NO: 501) corresponding to amino acids 1-350 of H13410_P9 (SEQ ID NO: 141), a second amino acid sequence being at least about 90% homologous to amino acids 1-586 of Q8N1G3_HUMAN (SEQ ID NO: 136), which also corresponds to amino acids 351-936 of H13410_P9 (SEQ ID NO: 141), and a third amino acid sequence being at least about 70%, optionally at least 80%, preferably at least 85%, more preferably at least 90% and most preferably at least 95% homologous to a polypeptide having the sequence YMLRYWSVGMDRVGLLIRENI (SEQ ID NO: 503) corresponding to amino acids 937-957 of H13410_P9 (SEQ ID NO: 141), wherein said first amino acid sequence, second amino acid sequence and third amino acid sequence are contiguous and in a sequential order.

In some embodiments, the isolated chimeric proteins or polypeptides of the invention may comprise an amino acid sequence corresponding to or homologous to that as set forth in H13410_P9 (SEQ ID NO: 141), comprising a first amino acid sequence being at least about 70%, optionally at least 80%, preferably at least 85%, more preferably at least 90% and most preferably at least 95%, homologous to a polypeptide having the sequence MAEPGHSHHLSARVRGRTERRIPRLWRLLLWAGTAFQVTQGTGPELHACKESEYHYEYTACDSTGSRWR VAVPHTPGLCTSLPDPVKGTECSFSCNAGEFLDMKDQSCKPCAEGRYSLGTGIRFDEWDELPHGFASLSAN MELDDSAAESTGNCTSSKWVPRGDYIASNTDECTATLMYAVNLKQSGTVNFEYYYPDSSIIFEFFVQNDQ CQPNADDSRWMKTTEKGWEFHSVELNRGNNVLYWRTTAFSVWTKVPKPVLVRNIAITGVAYTSECFPCK PGTYADKQGSSFCKLCPANSYSNKGETSCHQCDPDKYSEKGSSSCNVRPACTDKDYFYTHTACDANGET QL (SEQ ID NO: 501) corresponding to amino acids 1-350 of H13410_P9 (SEQ ID NO: 141), a second amino acid sequence being at least about 90% homologous to amino acids 1-272 of Q5T5C9_HUMAN (SEQ ID NO: 138), which also corresponds to amino acids 351-622 of H13410_P9 (SEQ ID NO: 141), a bridging amino acid P corresponding to amino acid 623 of H13410_P9 (SEQ ID NO: 141), a third amino acid sequence being at least about 90% homologous to amino acids 274-586 of Q5T5C9_HUMAN (SEQ ID NO: 138), which also corresponds to amino acids 624-936 of H13410_P9 (SEQ ID NO: 141), and a fourth amino acid sequence being at least about 70%, optionally at least 80%, preferably at least 85%, more preferably at least 90% and most preferably at least 95% homologous to a polypeptide having the sequence YMLRYWSVGMDRVGLLIRENI (SEQ ID NO: 503) corresponding to amino acids 937-957 of H13410_P9 (SEQ ID NO: 141), wherein said first amino acid sequence, second amino acid sequence, bridging amino acid, third amino acid sequence and fourth amino acid sequence are contiguous and in a sequential order.

In some embodiments, the isolated chimeric proteins or polypeptides of the invention may comprise an amino acid sequence corresponding to or homologous to that as set forth in H13410_P9 (SEQ ID NO: 141), comprising a first amino acid sequence being at least about 70%, optionally at least 80%, preferably at least 85%, more preferably at least 90% and most preferably at least 95%, homologous to a polypeptide having the sequence MEPGHSHHILSARVRGRTERRIPRLWRLLLWAGTAFQVTQGTGPELHACKESEYHYEYTACDSTGSRWR VAVPHTPGLCTSLPDPVKGTECSFSCNAGEFLDMKDQSCKPCAEGRYSLGTGIRFDEWDELPHGFASLSAN MELDDSAAESTGNCTSSKWVPRGDYIASNTDECTATLMYAVNLKQSGTVNFEYYYPDSSIIFEFFVQNDQ CQPNADDSRWMKTTEKGWEFHSVELNRGNNVLYWRTTAFSVWTKVPKPVLVRNIAITGVAYTSECFPCK PGTYADKQGSSFCKLCPANSYSNKGETSCHQCDPDKYSEKGSSSCNVRPACTDKDYFYTHTACDANGET QLMYKWAKPKICSEDLEGAVKLPASGVKTHCPPCNPGFFKTNNSTCQPCPYGSYSNGSDCTRCPAGTEPA VGFEYK (SEQ ID NO: 502) corresponding to amino acids 1-424 of H13410_P9 (SEQ ID NO: 141), a second amino acid sequence being at least about 90% homologous to amino acids 1-512 of Q9P2M2_HUMAN (SEQ ID NO: 135), which also corresponds to amino acids 425-936 of H13410_P9 (SEQ ID NO: 141), and a third amino acid sequence being at least about 70%, optionally at least 80%, preferably at least 85%, more preferably at least 90% and most preferably at least 95% homologous to a polypeptide having the sequence YMLRYWSVGMDRVGLLIRENI (SEQ ID NO: 503) corresponding to amino acids 937-957 of H13410_P9 (SEQ ID NO: 141), wherein said first amino acid sequence, second amino acid sequence and third amino acid sequence are contiguous and in a sequential order.

In some embodiments, this invention provides an isolated polypeptide encoding for a head of H13410_P9 (SEQ ID NO: 141), comprising a polypeptide being at least 70%, optionally at least about 80%, preferably at least about 85%, more preferably at least about 90% and most preferably at least about 95% homologous to the sequence

(SEQ ID NO: 502) of H13410_P9 (SEQ ID NO: 141)

MAEPGHSHHLSARVRGRTERRIPRLWRLLLWAGTAFQVTQGTGPELHACK
ESEYHYEYTACDSTGSRWRVAVPHTPGLCTSLPDPVKGTECSFSCNAGEF
LDMKDQSCKPCAEGRYSLGTGIRFDEWDELPHGFASLSANMELDDSAAES
TGNCTSSKWVPRGDYIASNTDECTATLMYAVNLKQSGTVNFEYYYPDSSI
IFEFFVQNDQCQPNADDSRWMKTTEKGWEFHSVELNRGNNVLYWRTTAFS
VWTKVPKPVLVRNIAITGVAYTSECFPCKPGTYADKQGSSFCKLCPANSY
SNKGETSCHQCDPDKYSEKGSSSCNVRPACTDKDYFYTHTACDANGETQL
MYKWAKPKICSEDLEGAVKLPASGVKTHCPPCNPGFFKTNNSTCQPCPYG
SYSNGSDCTRCPAGTEPAVGFEYK.

In some embodiments, such isolated chimeric proteins or polypeptides may comprise an amino acid sequence corresponding to or homologous to that set forth in H61775_P18 (SEQ ID NO:355).

In some embodiments, the isolated chimeric proteins or polypeptides of the invention may comprise an amino acid sequence corresponding to or homologous to that as set forth in H61775_P18 (SEQ ID NO:355), comprising a first amino acid sequence being at least about 90% homologous to amino acids 1-33 of NP_{—}065840 (SEQ ID NO: 351), which also corresponds to amino acids 1-33 of H61775_P18 (SEQ ID NO:355), a bridging amino acid G corresponding to amino acid 34 of H61775_P18 (SEQ ID NO:355), a second amino acid sequence being at least about 90% homologous to amino acids 35-83 of NP_{—}065840 (SEQ ID NO: 351), which also corresponds to amino acids 35-83 of H61775_P18 (SEQ ID NO:355), and a third amino acid sequence being at least about 70%, optionally at least 80%, preferably at least 85%, more preferably at least 90% and most preferably at least 95% homologous to a polypeptide having the sequence

(SEQ ID NO: 505)

KSSPQVGGREVSARKVGWVESHKASYERLCPCSDLISSSAALTLLNKHGC
APKQC

corresponding to amino acids 84-138 of H61775_P18 (SEQ ID NO:355), wherein said first amino acid sequence, bridging amino acid, second amino acid sequence and third amino acid sequence are contiguous and in a sequential order.

In some embodiments, this invention provides an isolated polypeptide encoding for an edge portion of H61775_P18 (SEQ ID NO:355), comprising an amino acid sequence being at least about 70%, optionally at least about 80%, preferably at least about 85%, more preferably at least about 90% and most preferably at least about 95% homologous to the sequence

(SEQ ID NO: 355)

KSSPQVGGREVSARKVGWVESHKASYERLCPCSDLISSSAALTLLNKHGC
APKQC of H61775_P18.

In some embodiments, the isolated chimeric proteins or polypeptides of the invention may comprise an amino acid sequence corresponding to or homologous to that as set forth in H61775_P18 (SEQ ID NO:355), comprising a first amino acid sequence being at least about 90% homologous to amino acids 11-93 of Q9P2J2_HUMAN (SEQ ID NO: 352), which also corresponds to amino acids 1-83 of H61775_P18 (SEQ ID NO:355), and a second amino acid sequence being at least about 70%, optionally at least 80%, preferably at least 85%, more preferably at least 90% and most preferably at least 95% homologous to a polypeptide having the sequence KSSPQVGGREVSARKVGWVESHKASYERLCPCSDLISSSAALTLLNKHGCAPKQC (SEQ ID NO: 505) corresponding to amino acids 84-138 of H61775_P18 (SEQ ID NO:355), wherein said, first amino acid sequence and second amino acid sequence are contiguous and in a sequential order.

In some embodiments, such isolated chimeric proteins or polypeptides may comprise an amino acid sequence corresponding to or homologous to that set forth in HUMPAX8A_P33 (SEQ ID NO: 183).

In some embodiments, the isolated chimeric proteins or polypeptides of the invention may comprise an amino acid sequence corresponding to or homologous to that as set forth in HUMPAX8A_P33 (SEQ ID NO: 183), comprising a first amino acid sequence being at least about 90% homologous to amino acids 1-201 of Q06710-5 (SEQ ID NO: 178), which also corresponds to amino acids 1-201 of HUMPAX8A_P33 (SEQ ID NO: 183), and a second amino acid sequence being at least about 70%, optionally at least 80%, preferably at least 85%, more preferably at least 90% and most preferably at least 95% homologous to a polypeptide having the sequence ECWDRRAAGGAREQFAQALMGTETLPLMIWDFVGALHVTVTCPVPQLAKAPGPTWLALFLPPQ (SEQ ID NO: 506) corresponding to amino acids 202-264 of HUMPAX8A_P33 (SEQ ID NO: 183), wherein said first amino acid sequence and second amino acid sequence are contiguous and in a sequential order.

In some embodiments, this invention provides an isolated polypeptide encoding for an edge portion of HUMPAX8A_P33 (SEQ ID NO: 183), comprising an amino acid sequence being at least about 70%, optionally at least about 80%, preferably at least about 85%, more preferably at least about 90% and most preferably at least about 95% homologous to the sequence

(SEQ ID NO: 506)

ECWDRRAAGGAREQFAQALMGTETLPLMIWDFVGALHVTVTCPVPQLAKA
PGPTWLALFLPPQ
of

(SEQ ID NO: 183)

HUMPAX8A_P33.

In some embodiments, the isolated chimeric proteins or polypeptides of the invention may comprise an amino acid sequence corresponding to or homologous to that as set forth in HUMPAX8A_P36 (SEQ ID NO: 184), comprising a first amino acid sequence being at least about 90% homologous to amino acids 1-259 of PAX8_HUMAN (SEQ ID NO:422), which also corresponds to amino acids 1-259 of HUMPAX8A_P36 (SEQ ID NO: 184), and a second amino acid sequence being at least about 70%, optionally at least 80%, preferably at least 85%, more preferably at least 90% and most preferably at least 95% homologous to a polypeptide having the sequence VRSWALGGEQGGQGPEKVYSPNEPVLHQSSWKLHQWAQGTCLMQLQA (SEQ ID NO: 507) corresponding to amino acids 260-306 of HUMPAX8A_P36 (SEQ ID NO: 184), wherein said first amino acid sequence and second amino acid sequence are contiguous and in a sequential order.

In some embodiments, this invention provides an isolated polypeptide encoding for an edge portion of HUMPAX8A_P36 (SEQ ID NO: 184), comprising an amino acid sequence being at least about 70%, optionally at least about 80%, preferably at least about 85%, more preferably at least about 90% and most preferably at least about 95% homologous to the sequence

(SEQ ID NO: 507)

VRSWALGGEQGGQGPEKVYSPNEPVLHQSSWKLHQWAQGTCLMQLQA
of

(SEQ ID NO: 184)

HUMPAX8A_P36.

In some embodiments, such isolated chimeric proteins or polypeptides may comprise an amino acid sequence corresponding to or homologous to that set forth in HUMPAX8A_P37 (SEQ ID NO: 185).

In some embodiments, the isolated chimeric proteins or polypeptides of the invention may comprise an amino acid sequence corresponding to or homologous to that as set forth in HUMPAX8A_P37 (SEQ ID NO: 185), comprising a first amino acid sequence being at least about 90% homologous to amino acids 1-63 of PAX8_HUMAN (SEQ ID NO:422), which also corresponds to amino acids 1-63 of HUMPAX8A_P37 (SEQ ID NO: 185), a bridging amino acid S corresponding to amino acids 64 4 of HUMPAX8A_P37 (SEQ ID NO: 185), a second amino acid sequence being at least about 90% homologous to amino acids 64-259 of PAX8_HUMAN (SEQ ID NO:422), which also corresponds to amino acids 65-260 of HUMPAX8A_P37 (SEQ ID NO: 185), and a third amino acid sequence being at least about 70%, optionally at least 80%, preferably at least 85%, more preferably at least 90% and most preferably at least 95% homologous to a polypeptide having the sequence VRSWALGGEQGGQGPEKVYSPNEPVLHQSSWKLHQWAQGTCLMQLQA (SEQ ID NO: 507) corresponding to amino acids 261-307 of HUMPAX8A_P37 (SEQ ID NO: 185), wherein said first amino acid sequence, bridging amino acid, second amino acid sequence, and third amino acid sequence are contiguous and in a sequential order.

In some embodiments, such isolated chimeric proteins or polypeptides may comprise an amino acid sequence corresponding to or homologous to that set forth in T58132_P0 (SEQ ID NO: 213).

In some embodiments, the isolated chimeric proteins or polypeptides of the invention may comprise an amino acid sequence corresponding to or homologous to that as set forth in T58132_P0 (SEQ ID NO: 213), comprising a first amino acid sequence being at least about 70%, optionally at least 80%, preferably at least 85%, more preferably at least 90% and most preferably at least 95%, homologous to a polypeptide having the sequence AAWKAGSGSAGNCEADCEGKGALLSPPGFYHQKRGQTSII (SEQ ID NO: 508) corresponding to amino acids 1-40 of T58132_P0 (SEQ ID NO: 213), and a second amino acid sequence being at least about 90% homologous to amino acids 1-181 of Q6L9U4_HUMAN (SEQ ID NO:425), which also corresponds to amino acids 41-221 of T58132_P0 (SEQ ID NO: 213), wherein said first amino acid sequence and second amino acid sequence are contiguous and in a sequential order.

In some embodiments, this invention provides an isolated polypeptide encoding for a head of T58132_P0 (SEQ ID NO: 213), comprising a polypeptide being at least 70%, optionally at least about 80%, preferably at least about 85%, more preferably at least about 90% and most preferably at least about 95% homologous to the sequence

(SEQ ID NO: 508)

AAWKAGSGSAGNCEADCEGKGALLSPPGFYHQKRGQTSII
of

(SEQ ID NO: 213)

T58132_P0.

In some embodiments, the isolated chimeric proteins or polypeptides of the invention may comprise an amino acid sequence corresponding to or homologous to that as set forth in T58132_P0 (SEQ ID NO: 213), comprising a first amino acid sequence being at least about 70%, optionally at least 80%, preferably at least 85%, more preferably at least 90% and most preferably at least 95%, homologous to a polypeptide having the sequence AAWKAGSGSAGNCEADCEGKGALLSPPGFYHQKRGQTSII (SEQ ID NO: 508) corresponding to amino acids 1-40 of T58132_P0 (SEQ ID NO: 213), a second amino acid sequence being at least about 90% homologous to amino acids 1-82 of Q68G75_HUMAN (SEQ ID NO: 209), which also corresponds to amino acids 41-122 of T58132_P0 (SEQ ID NO: 213), a bridging amino acid E corresponding to amino acid 123 of T58132_P0 (SEQ ID NO: 213), and a third amino acid sequence being at least about 90% homologous to amino acids 84-181 of Q68G75_HUMAN (SEQ ID NO: 209), which also corresponds to amino acids 124-221 of T58132_P0 (SEQ ID NO: 213), wherein said first amino acid sequence, second amino acid sequence, bridging amino acid and third amino acid sequence are contiguous and in a sequential order.

In some embodiments, the isolated chimeric proteins or polypeptides of the invention may comprise an amino acid sequence corresponding to or homologous to that as set forth in T58132_P0 (SEQ ID NO: 213), comprising a first amino acid sequence being at least about 70%, optionally at least 80%, preferably at least 85%, more preferably at least 90% and most preferably at least 95%, homologous to a polypeptide having the sequence AAWKAGSGSAGNCEADCEGKGALLSPPGFYHQKRGQTSII (SEQ ID NO: 508) corresponding to amino acids 1-40 of T58132_P0 (SEQ ID NO: 213), a second amino acid sequence being at least about 90% homologous to amino acids 1-27 of Q6L9U2_HUMAN (SEQ ID NO: 212), which also corresponds to amino acids 41-67 of T58132_P0 (SEQ ID NO: 213), a third amino acid sequence being at least about 70%, optionally at least 80%, preferably at least 85%, more preferably at least 90% and most preferably at least 95% homologous to a polypeptide having the sequence PSTRKLYEKKLVQLLVSPPCAPPVMNGPRELDGAQDSDDSEE (SEQ ID NO: 509) corresponding to amino acids 68-109 of T58132_P0 (SEQ ID NO: 213), and a fourth amino acid sequence being at least about 90% homologous to amino acids 29-140 of Q6L9U2_HUMAN (SEQ ID NO: 212), which also corresponds to amino acids 110-221 of T58132_P0 (SEQ ID NO: 213), wherein said first amino acid sequence, second amino acid sequence, third amino acid sequence and fourth amino acid sequence are contiguous and in a sequential order.

In some embodiments, this invention provides an isolated polypeptide encoding for an edge portion of T58132_P0 (SEQ ID NO: 213), comprising an amino acid sequence being at least about 70%, optionally at least about 80%, preferably at least about 85%, more preferably at least about 90% and most preferably at least about 95% homologous to the sequence PSTRKLYEKKLVQLLVSPPCAPPVMNGPRELDGAQDSDDSEE (SEQ ID NO: 509) of T58132_P0 (SEQ ID NO: 213).

In some embodiments, such isolated chimeric proteins or polypeptides may comprise an amino acid sequence corresponding to or homologous to that set forth in T58132_P1 (SEQ ID NO: 214).

In some embodiments, the isolated chimeric proteins or polypeptides of the invention may comprise an amino acid sequence corresponding to or homologous to that as set forth in T58132_P1 (SEQ ID NO: 214), comprising a first amino acid sequence being at least about 70%, optionally at least 80%, preferably at least 85%, more preferably at least 90% and most preferably at least 95%, homologous to a polypeptide having the sequence AAWKAGSGSAGNCEADCEGKGALLSPPGFYHQKRGQTSII (SEQ ID NO: 508) corresponding to amino acids 1-40 of T58132_P1 (SEQ ID NO: 214), and a second amino acid sequence being at least about 90% homologous to amino acids 1-108 of NP_{—}001001552 (SEQ ID NO: 206), which also corresponds to amino acids 41-148 of T58132_P1 (SEQ ID NO: 214), wherein said first amino acid sequence and second amino acid sequence are contiguous and in a sequential order.

In some embodiments, the isolated chimeric proteins or polypeptides of the invention may comprise an amino acid sequence corresponding to or homologous to that as set forth in T58132_P12 (SEQ ID NO: 216), comprising a first amino acid sequence being at least about 90% homologous to amino acids 1-27 of Q6L9U4_HUMAN (SEQ ID NO:425), which also corresponds to amino acids 1-27 of T58132_P12 (SEQ ID NO: 216), a second bridging amino acid sequence comprising of Q, and a third amino acid sequence being at least about 90% homologous to amino acids 70-181 of Q6L9U4 HUMAN (SEQ ID NO:425), which also corresponds to amino acids 29-140 of T58132_P12 (SEQ ID NO: 216), wherein said first amino acid sequence, second amino acid sequence and third amino acid sequence are contiguous and in a sequential order.

In some embodiments, this invention provides an isolated polypeptide encoding for an edge portion of T58132_P12 (SEQ ID NO: 216), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least 3 amino acids comprise LQL having a structure as follows (numbering according to T58132_P12 (SEQ ID NO: 216)): a sequence starting from any of amino acid numbers 27−x to 27; and ending at any of amino acid numbers 29+((n−3)−x), in which x varies from 0 to n−3.

In some embodiments, the isolated chimeric proteins or polypeptides of the invention may comprise an amino acid sequence corresponding to or homologous to that as set forth in T58132_P12 (SEQ ID NO: 216), comprising a first amino acid sequence being at least about 90% homologous to amino acids 1-27 of Q68G75_HUMAN (SEQ ID NO: 209), which also corresponds to amino acids 1-27 of T58132_P12 (SEQ ID NO: 216), a second bridging amino acid sequence comprising of Q, a third amino acid sequence being at least about 90% homologous to amino acids 70-82 of Q68G75_HUMAN (SEQ ID NO: 209), which also corresponds to amino acids 29-41 of T58132_P12 (SEQ ID NO: 216), a bridging amino acid E corresponding to amino acid 42 of T58132_P12 (SEQ ID NO: 216), and a fourth amino acid sequence being at least about 90% homologous to amino acids 84-181 of Q68G75_HUMAN (SEQ ID NO: 209), which also corresponds to amino acids 43-140 of T58132_P12 (SEQ ID NO: 216), wherein said first amino acid sequence, second amino acid sequence, third amino acid sequence, bridging amino acid and fourth amino acid sequence are contiguous and in a sequential order.

In some embodiments, such isolated chimeric proteins or polypeptides may comprise an amino acid sequence corresponding to or homologous to that set forth in T86235_P4 (SEQ ID NO:469).

In some embodiments, the isolated chimeric proteins or polypeptides of the invention may comprise an amino acid sequence corresponding to or homologous to that as set forth in T86235_P4 (SEQ ID NO:469), comprising a first amino acid sequence being at least about 90% homologous to amino acids 1-223 of TAST_HUMAN (SEQ ID NO: 463), which also corresponds to amino acids 1-223 of T86235_P4 (SEQ ID NO:469), a bridging amino acid R corresponding to amino acid 224 of T86235_P4 (SEQ ID NO:469), a second amino acid sequence being at least about 90% homologous to amino acids 225-388 of TAST_HUMAN (SEQ ID NO: 463), which also corresponds to amino acids 225-388 of T86235_P4 (SEQ ID NO:469), and a third amino acid sequence being at least about 70%, optionally at least 80%, preferably at least 85%, more preferably at least 90% and most preferably at least 95% homologous to a polypeptide having the sequence VSIRSFPLLRSLALCCQPGGPGV (SEQ ID NO: 510) corresponding to amino acids 389-411 of T86235_P4 (SEQ ID NO:469), wherein said first amino acid sequence, bridging amino acid, second amino acid sequence and third amino acid sequence are contiguous and in a sequential order.

In some embodiments, this invention provides an isolated polypeptide encoding for an edge portion of T86235_P4 (SEQ ID NO:469), comprising an amino acid sequence being at least about 70%, optionally at least about 80%, preferably at least about 85%, more preferably at least about 90% and most preferably at least about 95% homologous to the sequence VSIRSFPLLRSLALCCQPGGPGV (SEQ ID NO: 510) of T86235_P4 (SEQ ID NO:469).

In some embodiments, the isolated chimeric proteins or polypeptides of the invention may comprise an amino acid sequence corresponding to or homologous to that as set forth in T86235_P4 (SEQ ID NO:469), comprising a first amino acid sequence being at least about 90% homologous to amino acids 1-388 of Q8N5B2_HUMAN (SEQ ID NO: 467), which also corresponds to amino acids 1-388 of T86235_P4 (SEQ ID NO:469), and a second amino acid sequence being at least about 70%, optionally at least 80%, preferably at least 85%, more preferably at least 90% and most preferably at least 95% homologous to a polypeptide having the sequence VSIRSFPLLRSLALCCQPGGPGV (SEQ ID NO: 510) corresponding to amino acids 389-411 of T86235_P4 (SEQ ID NO:469), wherein said first amino acid sequence and second amino acid sequence are contiguous and in a sequential order.

In some embodiments, the isolated chimeric proteins or polypeptides of the invention may comprise an amino acid sequence corresponding to or homologous to that as set forth in T86235_P4 (SEQ ID NO:469), comprising a first amino acid sequence being at least about 90% homologous to amino acids 1-112 of Q6PJU7_HUMAN (SEQ ID NO: 439), which also corresponds to amino acids 1-112 of T86235_P4 (SEQ ID NO:469), and a second amino acid sequence being at least about 70%, optionally at least 80%, preferably at least 85%, more preferably at least 90% and most preferably at least 95% homologous to a polypeptide having the sequence EAPGTIEFVADPAALATILSGEGVKSCHLGRQPSLAKRVLVRGSQGGTTQRVQGVRASAYLAPRTPTHRLD PARASCFSRLEGPGPRGRTLCPQRLQALISPSGPSFHPSTRPSFQELRRETAGSSRTSVSQASGLLLETPVQPA FSLPKGEREVVTHSDEGGVASLGLAQRVPLRENREMSHTRDSHDSHLMPSPAPVAQPLPGHVVPCPSPFGR AQRVPSPGPPTLTSYSVLRRLTVQPKTRFTPMPSTPRVQQAQWLRGVSPQSCSEDPALPWVSIRSFPLLRSL ALCCQPGGPGV (SEQ ID NO: 511) corresponding to amino acids 113-411 of T86235_P4 (SEQ ID NO:469), wherein said first amino acid sequence and second amino acid sequence are contiguous and in a sequential order.

In some embodiments, this invention provides an isolated polypeptide encoding for an edge portion of T86235_P4 (SEQ ID NO:469), comprising an amino acid sequence being at least about 70%, optionally at least about 80%, preferably at least about 85%, more preferably at least about 90% and most preferably at least about 95% homologous to the sequence

(SEQ ID NO: 511)

EAPGTIEFVADPAALATILSGEGVKSCHLGRQPSLAKRVLVRGSQGGTTQ
RVQGVRASAYLAPRTPTHRLDPARASCFSRLEGPGPRGRTLCPQRLQALI
SPSGPSFHPSTRPSFQELRRETAGSSRTSVSQASGLLLETPVQPAFSLPK
GEREVVTHSDEGGVASLGLAQRVPLRENREMSHTRDSHDDHLMPSPAPVA
QPLPGHVVPCPSPFGRAQRVPSPGPPTLTSYSVLRRLTVQPKTRFTPMPS
TPRVQQAQWLRGVSPQSCSEDPALPWVSIRSFPLLRSLALCCQPGGPGV
of

(SEQ ID NO: 469)

T86235_P4.

In some embodiments, such isolated chimeric proteins or polypeptides may comprise an amino acid sequence corresponding to or homologous to that set forth in T86235_P5 (SEQ ID NO:470).

In some embodiments, the isolated chimeric proteins or polypeptides of the invention may comprise an amino acid sequence corresponding to or homologous to that as set forth in T86235_P5 (SEQ ID NO:470), comprising a first amino acid sequence being at least about 90% homologous to amino acids 1-223 of TAST_HUMAN (SEQ ID NO: 463), which also corresponds to amino acids 1-223 of T86235_P5 (SEQ ID NO:470), a bridging amino acid R corresponding to amino acid 224 of T86235_P5 (SEQ ID NO:470), a second amino acid sequence being at least about 90% homologous to amino acids 225-433 of TAST_HUMAN (SEQ ID NO: 463), which also corresponds to amino acids 225-433 of T86235_P5 (SEQ ID NO:470), and a third amino acid sequence being at least about 70%, optionally at least 80%, preferably at least 85%, more preferably at least 90% and most preferably at least 95% homologous to a polypeptide having the sequence VSLCGQQL (SEQ ID NO: 512) corresponding to amino acids 434-441 of T86235_P5 (SEQ ID NO:470), wherein said first amino acid sequence, bridging amino acid, second amino acid sequence and third amino acid sequence are contiguous and in a sequential order.

In some embodiments, this invention provides an isolated polypeptide encoding for an edge portion of T86235_P5 (SEQ ID NO:470), comprising an amino acid sequence being at least about 70%, optionally at least about 80%, preferably at least about 85%, more preferably at least about 90% and most preferably at least about 95% homologous to the sequence VSLCGQQL (SEQ ID NO: 512) of T86235_P5(SEQ ID NO:470).

In some embodiments, the isolated chimeric proteins or polypeptides of the invention may comprise an amino acid sequence corresponding to or homologous to that as set forth in T86235_P5 (SEQ ID NO:470), comprising a first amino acid sequence being at least about 90% homologous to amino acids 1-433 of Q8N5B2_HUMAN (SEQ ID NO: 467), which also corresponds to amino acids 1-433 of T86235_P5 (SEQ ID NO:470), and a second amino acid sequence being at least about 70%, optionally at least 80%, preferably at least 85%, more preferably at least 90% and most preferably at least 95% homologous to a polypeptide having the sequence VSLCGQQL (SEQ ID NO: 512) corresponding to amino acids 434-441 of T86235_P5 (SEQ ID NO:470), wherein said first amino acid sequence and second amino acid sequence are contiguous and in a sequential order.

In some embodiments, the isolated chimeric proteins or polypeptides of the invention may comprise an amino acid sequence corresponding to or homologous to that as set forth in T86235_P5 (SEQ ID NO:470), comprising a first amino acid sequence being at least about 90% homologous to amino acids 1-112 of Q6PJU7_HUMAN (SEQ ID NO: 439), which also corresponds to amino acids 1-112 of T86235_P5 (SEQ ID NO:470), and a second amino acid sequence being at least about 70%, optionally at least 80%, preferably at least 85%, more preferably at least 90% and most preferably at least 95% homologous to a polypeptide having the sequence EAPGTIEFVADPAALATILSGEGVKSCHLGRQPSLAKRVLVRGSQGGTTQRVQGVRASAYLAPRTPTHRLD PARASCFSRLEGPGPRGRTLCPQRLQALISPSGPSFHPSTRPSFQELRRETAGSSRTSVSQASGLLLETPVQPA FSLPKGEREVVTHSDEGGVASLGLAQRVPLRENREMSHTRDSHDSHLMPSPAPVAQPLPGHVVPCPSPFGR AQRVPSPGPPTLTSYSVLRRLTVQPKTRFTPMPSTPRVQQAQWLRGVSPQSCSEDPALPWEQVAVRLFDQE SCIRSLEGSGKPPVATPSGPHSNRTPSLQEVKIQVSLCGQQL (SEQ ID NO: 513) corresponding to amino acids 113-441 of T86235_P5 (SEQ ID NO:470), wherein said first amino acid sequence and second amino acid sequence are contiguous and in a sequential order.

In some embodiments, this invention provides an isolated polypeptide encoding for an edge portion of T86235_P5 (SEQ ID NO:470), comprising an amino acid sequence being at least about 70%, optionally at least about 80%, preferably at least about 85%, more preferably at least about 90% and most preferably at least about 95% homologous to the sequence

(SEQ ID NO: 513)

EAPGTIEFVADPAALATILSGEGVKSCHLGRQPSLAKRVLVRGSQGGTTQ
RVQGVRASAYLAPRTPTHRLDPARASCFSRLEGPGPRGRTLCPQRLQALI
SPSGPSFHPSTRPSFQELRRETAGSSRTSVSQASGLLLETPVQPAFSLPK
GEREVVTHSDEGGVASLGLAQRVPLRENREMSHTRDSHDSHLMPSPAPVA
QPLPGHVVPCPSPFGRAQRVPSPGPPTLTSYSVLRRLTVQPKTRFTPMPS
TPRVQQAQWLRGVSPQSCSEDPALPWEQVAVRLFDQESCIRSLEGSGKPP
VATPSGPHSNRTPSLQEVKIQVSLCGQQL
of

(SEQ ID NO: 470)

T86235_P5.

In some embodiments, such isolated chimeric proteins or polypeptides may comprise an amino acid sequence corresponding to or homologous to that set forth in T86235_P6 (SEQ ID NO:471).

In some embodiments, the isolated chimeric proteins or polypeptides of the invention may comprise an amino acid sequence corresponding to or homologous to that as set forth in T86235_P6 (SEQ ID NO:471), comprising a first amino acid sequence being at least about 90% homologous to amino acids 1-223 of TAST_HUMAN (SEQ ID NO: 463), which also corresponds to amino acids 1-223 of T86235_P6 (SEQ ID NO:471), a bridging amino acid R corresponding to amino acid 224 of T86235_P6 (SEQ ID NO:471), a second amino acid sequence being at least about 90% homologous to amino acids 225-576 of TAST_HUMAN (SEQ ID NO: 463), which also corresponds to amino acids 225-576 of T86235_P6 (SEQ ID NO:471), a third amino acid sequence being at least about 90% homologous to amino acids 606-699 of TAST_HUMAN (SEQ ID NO: 463), which also corresponds to amino acids 577-670 of T86235_P6 (SEQ ID NO:471), a fourth amino acid sequence being at least about 70%, optionally at least 80%, preferably at least 85%, more preferably at least 90% and most preferably at least 95% homologous to a polypeptide having the sequence GKELAGKECEHKRSSPHCELHTCPAPTPGNLMLPHLPPWPSLALPQEEGRGCTSSPVLLIGLAVGGGGGED STWWKYRTPDLPLNFPCPS (SEQ ID NO: 514) corresponding to amino acids 671-760 of T86235_P6 (SEQ ID NO:471), and a fifth amino acid sequence being at least about 90% homologous to amino acids 700-778 of TAST_HUMAN (SEQ ID NO: 463), which also corresponds to amino acids 761-839 of T86235_P6 (SEQ ID NO:471), wherein said first amino acid sequence, bridging amino acid, second amino acid sequence, third amino acid sequence, fourth amino acid sequence and fifth amino acid sequence are contiguous and in a sequential order.

In some embodiments, the isolated chimeric proteins or polypeptides of the invention may comprise an amino acid sequence corresponding to or homologous to that as set forth in an edge portion of T86235_P6 (SEQ ID NO:471), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EE, having a structure as follows: a sequence starting from any of amino acid numbers 576−x to 576; and ending at any of amino acid numbers 577+((n−2)−x), in which x varies from 0 to n−2.

In some embodiments, this invention provides an isolated polypeptide encoding for an edge portion of T86235_P6 (SEQ ID NO:471), comprising an amino acid sequence being at least about 70%, optionally at least about 80%, preferably at least about 85%, more preferably at least about 90% and most preferably at least about 95% homologous to the sequence

(SEQ ID NO: 514)

GKELAGKECEHKRSSPHCELHTCPAPTPGNLMLPHIPPWPSLALPQEEGR
GCTSSPVLLIGLAVGGGGGEDSTWWKYRTPDLPLNFPCPS
of

(SEQ ID NO: 471)

T86235_P6.

In some embodiments, the isolated chimeric proteins or polypeptides of the invention may comprise an amino acid sequence corresponding to or homologous to that as set forth in T86235_P6 (SEQ ID NO:471), comprising a first amino acid sequence being at least about 90% homologous to amino acids 1-576 of Q8N5B2_HUMAN (SEQ ID NO: 467), which also corresponds to amino acids 1-576 of T86235_P6 (SEQ ID NO:471), a second amino acid sequence being at least about 90% homologous to amino acids 606-699 of Q8N5B2_HUMAN (SEQ ID NO: 467), which also corresponds to amino acids 577-670 of T86235_P6 (SEQ ID NO:471), a third amino acid sequence being at least about 70%, optionally at least 80%, preferably at least 85%, more preferably at least 90% and most preferably at least 95% homologous to a polypeptide having the sequence GKELAGKECEHKRSSPHCELHTCPAPTPGNLMLPHLPPWPSLALPQEEGRGCTSSPVLLIGLAVGGGGGED STWWKYRTPDLPLNFPCPS (SEQ ID NO: 514) corresponding to amino acids 671-760 of T86235_P6 (SEQ ID NO:471), and a fourth amino acid sequence being at least about 90% homologous to amino acids 700-778 of Q8N5B2_HUMAN (SEQ ID NO: 467), which also corresponds to amino acids 761-839 of T86235_P6 (SEQ ID NO:471), wherein said first amino acid sequence, second amino acid sequence, third amino acid sequence and fourth amino acid sequence are contiguous and in a sequential order.

In some embodiments, the isolated chimeric proteins or polypeptides of the invention may comprise an amino acid sequence corresponding to or homologous to that as set forth in an edge portion of T86235_P6 (SEQ ID NO:471), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EE, having a structure as follows: a sequence starting from any of amino acid numbers 576−x to 576; and ending at any of amino acid numbers 577+((n−2)−x), in which x varies from 0 to n−2.

In some embodiments, the isolated chimeric proteins or polypeptides of the invention may comprise an amino acid sequence corresponding to or homologous to that as set forth in T86235_P6 (SEQ ID NO:471), comprising a first amino acid sequence being at least about 90% homologous to amino acids 1-112 of Q6PJU7_HUMAN (SEQ ID NO: 439), which also corresponds to amino acids 1-112 of T86235_P6 (SEQ ID NO:471), and a second amino acid sequence being at least about 70%, optionally at least 80%, preferably at least 85%, more preferably at least 90% and most preferably at least 95% homologous to a polypeptide having the sequence EAPGTIEFVADPAALATILSGEGVKSCHLGRQPSLAKRVLVRGSQGGTTQRVQGVRASAYLAPRTPTHRLD PARASCFSRLEGPGPRGRTLCPQRLQALISPSGPSFHPSTRPSFQELRRETAGSSRTSVSQASGLLLETPVQPA FSLPKGEREVVTHSDEGGVASLGLAQRVPLRENREMSHTRDSHDSHLMPSPAPVAQPLPGHVVPCPSPFGR AQRVPSPGPPTLTSYSVLRRLTVQPKTRFTPMPSTPRVQQAQWLRGVSPQSCSEDPALPWEQVAVRLFDQE SCIRSLEGSGKPPVATPSGPHSNRTPSLQEVKIQRIGILQQLLRQEVEGLVGGQCVPLNGGSSLDMVELQPL LTEISRTLNATEHNSGTSHLPGLLKHSGLPKPCLPEECGEPQPCPPAEPGPPEAFCRSEPEIPEPSLQEQLEVP EPYPPAEPRPLESCCRSEPEIPESSRQEQLEEQLEVPEPCPPAEPGPLQPSTQGQSGPPGPCPRVELGASEPCT LEHRSLESSLPPCCSQWAPATTSLIFSSQHPLCASPPICSLQSLRPPAGQAGKELAGKECEHKRSSPHCELHT CPAPTPGNLMLPHLPPWPSLALPQEEGRGCTSSPVLLIGLAVGGGGGEDSTWWKYRTPDLPLNFPCPSGLS NLAPRTLALRERLKSCLTAIHCFBEARLDDECAFYTSRAPPSGPTRVCTNPVATLLEWQDALCFIPVGSAAP QGSP (SEQ ID NO: 515) corresponding to amino acids 113-839 of T86235_P6 (SEQ ID NO:471), wherein said first amino acid sequence and second amino acid sequence are contiguous and in a sequential order.

In some embodiments, this invention provides an isolated polypeptide encoding for an edge portion of T86235_P6 (SEQ ID NO:471), comprising an amino acid sequence being at least about 70%, optionally at least about 80%, preferably at least about 85%, more preferably at least about 90% and most preferably at least about 95% homologous to the sequence

(SEQ ID NO: 515)

EAPGTIEFVADPAALATILSGEGVKSCHLGRQPSLAKRVLVRGSQGGTTQ
RVQGVRASAYLAPRTPTHRLDPARASCFSRLEGPGPRGRTLCPQRLQALI
SPSGPSFHPSTRPSFQELRRETAGSSRTSVSQASGLLLETPVQPAFSLPK
GEREVVTHSDEGGVASLGLAQRVPLRENREMSHTRDSHDSHLMPSPAPVA
QPLPGHVVPCPSPFGRAQRVPSPGPPTLTSYSVLRRLTVQPKTRFTPMPS
TPRVQQAQWLRGVSPQSCSEDPALPWEQVAVRLFDQESCIRSLEGSGKPP
VATPSGPHSNRTPSLQEVKIQRIGILQQLLRQEVEGLVGGQCVPLNGGSS
LDMVELQPLLTEISRTLNATEHNSGTSHLPGLLKHSGLPKPCLPEECGEP
QPCPPAEPGPPEAFCRSEPEIPEPSLQEQLEVPEPYPPAEPRPLESCCRS
EPEIPESSRQEQLEEQLEVPEPCPPAEPGPLQPSTQGQSGPPGPCPRVEL
GASEPCTLEHRSLESSLPPCCSQWAPATTSLIFSSQHPLCASPPICSLQS
LRPPAGQAGKELAGKECEHKRSSPHCELHTCPAPTPGNLMLPHLPPWPSL
ALPQEEGRGCTSSPVLLIGLAVGGGGGEDSTWWKYRTPDLPLNFPCPSGL
SNLAPRTLALRERLKSCLTAIHCFHEARLDDECAFYTSRAPPSGPTRVCT
NPVATLLEWQDALCFIPVGSAAPQGSP
of

(SEQ ID NO: 471)

T86235_P6.

In some embodiments, the isolated chimeric proteins or polypeptides of the invention may comprise an amino acid sequence corresponding to or homologous to that as set forth in T86235_P7 (SEQ ID NO:472), comprising a first amino acid sequence being at least about 90% homologous to amino acids 1-223 of TAST_HUMAN (SEQ ID NO: 463), which also corresponds to amino acids 1-223 of T86235_P7 (SEQ ID NO:472), a bridging amino acid R corresponding to amino acid 224 of T86235_P7 (SEQ ID NO:472), a second amino acid sequence being at least about 90% homologous to amino acids 225-699 of TAST_HUMAN (SEQ ID NO: 463), which also corresponds to amino acids 225-699 of T86235_P7 (SEQ ID NO:472), a third amino acid sequence being at least about 70%, optionally at least 80%, preferably at least 85%, more preferably at least 90% and most preferably at least 95% homologous to a polypeptide having the sequence GKELAGKECEHKRSSPHCELHTCPAPTPGNLMLPHLPPWPSLALPQEEGRGCTSSPVLLIGLAVGGGGGED STWWKYRTPDLPLNFPCPS (SEQ ID NO: 514) corresponding to amino acids 700-789 of T86235_P7 (SEQ ID NO:472), and a fourth amino acid sequence being at least about 90% homologous to amino acids 700-778 of TAST_HUMAN (SEQ ID NO: 463), which also corresponds to amino acids 790-868 of T86235_P7 (SEQ ID NO:472), wherein said first amino acid sequence, bridging amino acid, second amino acid sequence, third amino acid sequence and fourth amino acid sequence are contiguous and in a sequential order.

In some embodiments, this invention provides an isolated polypeptide encoding for an edge portion of T86235_P7 (SEQ ID NO:472), comprising an amino acid sequence being at least about 70%, optionally at least about 80%, preferably at least about 85%, more preferably at least about 90% and most preferably at least about 95% homologous to the sequence

(SEQ ID NO: 514)

GKELAGKECEHKRSSPHCELHTCPAPTPGNLMLPHLPPWPSLALPQEEGR
GCTSSPVLLIGLAVGGGGGEDSTWWKYRTPDLPLNFPCPS
of

(SEQ ID NO: 472)

T86235_P7.

In some embodiments, the isolated chimeric proteins or polypeptides of the invention may comprise an amino acid sequence corresponding to or homologous to that as set forth in T86235_P7 (SEQ ID NO:472), comprising a first amino acid sequence being at least about 90% homologous to amino acids 1-699 of Q8N5B2_HUMAN (SEQ ID NO: 467), which also corresponds to amino acids 1-699 of T86235_P7 (SEQ ID NO:472), a second amino acid sequence being at least about 70%, optionally at least 80%, preferably at least 85%, more preferably at least 90% and most preferably at least 95% homologous to a polypeptide having the sequence GKELAGKECEHKRSSPHCELHTCPAPTPGNLMLPHLPPWPSLALPQEEGRGCTSSPVLLIGLAVGGGGGED STWWKYRTPDLPLNFPCPS (SEQ ID NO: 514) corresponding to amino acids 700-789 of T86235_P7 (SEQ ID NO:472), and a third amino acid sequence being at least about 90% homologous to amino acids 700-778 of known protein(s) □8N5B2_HUMAN (SEQ ID NO: 467), which also corresponds to amino acids 790-868 of T86235_P7 (SEQ ID NO:472), wherein said first amino acid sequence, second amino acid sequence and third amino acid sequence are contiguous and in a sequential order.

In some embodiments, this invention provides an isolated polypeptide encoding for an edge portion of T86235_P7 (SEQ ID NO:472), comprising an amino acid sequence being at least about 70%, optionally at least about 80%, preferably at least about 85%, more preferably at least about 90% and most preferably at least about 95% homologous to the sequence

(SEQ ID NO: 514)

GKELAGKECEHKRSSPHCELHTCPAPTPGNLMLPHLPPWPSLALPQEEGR
GCTSSPVLLIGLAVGGGGGEDSTWWKYRTPDLPLNFPCPS
of

(SEQ ID NO: 472)

T86235_P7.

In some embodiments, the isolated chimeric proteins or polypeptides of the invention may comprise an amino acid sequence corresponding to or homologous to that as set forth in T86235_P7 (SEQ ID NO:472), comprising a first amino acid sequence being at least about 90% homologous to amino acids 1-112 of Q6PJU7_HUMAN (SEQ ID NO: 439), which also corresponds to amino acids 1-112 of T86235_P7 (SEQ ID NO:472), and a second amino acid sequence being at least about 70%, optionally at least 80%, preferably at least 85%, more preferably at least 90% and most preferably at least 95% homologous to a polypeptide having the sequence EAPGTIEFVADPAALATILSGEGVKSCHLGRQPSLAKRVLVRGSQGGTTQRVQGVRASAYLAPRTPTHRLD PARASCFSRLEGPGPRGRTLCPQRLQALISPSGPSFHPSTRPSFQELRRETAGSSRTSVSQASGLLLETPVQPA FSLPKGEREVVTHSDEGGVASLGLAQRVPLRENREMSHTRDSHDSHLMPSPAPVAQPLPGHVVPCPSPFGR AQRVPSPGPPTLTSYSVLRRLTVQPKTRFTPMPSTPRVQQAQWLRGVSPQSCSEDPALPWEQVAVRLFDQE SCIRSLEGSGKPPVATPSGPHSNRTPSLQEVKIQRIGILQQLLRQEVEGLVGGQCVPLNGGSSLDMVELQPL LTEISRTLNATEHNSGTSHLPGLLKHSGLPKPCLPEECGEPQPCPPAEPGPPEAFCRSEPEIPEPSLQEQLEVP EPYPPAEPRPLESCCRSEPEIPESSRQEQLEVPEPCPPAEPRPLESYCRIEPEIPESSRQEQLEVPEPCPPAEPGP LQPSTQGQSGPPGPCPRVELGASEPCTLEHRSLESSLPPCCSQWAPATTSLIFSSQHPLCASPPICSLQSLRPP AGQAGKELAGKECEHKRSSPHCELHTCPAPTPGNLMLPHLPPWPSLALPQEEGRGCTSSPVLLIGLAVGGG GGEDSTWWKYRTPDLPLNFPCPSGLSNLAPRTLALRERLKSCLTAIHCFHEARLDDECAFYTSRAPPSGPT RVCTNPVATLLEWQDALCFIPVGSAAPQGSP (SEQ ID NO: 517) corresponding to amino acids 113-868 of T86235_P7 (SEQ ID NO:472), wherein said first amino acid sequence and second amino acid sequence are contiguous and in a sequential order.

In some embodiments, this invention provides an isolated polypeptide encoding for an edge portion of T86235_P7 (SEQ ID NO:472), comprising an amino acid sequence being at least about 70%, optionally at least about 80%, preferably at least about 85%, more preferably at least about 90% and most preferably at least about 95% homologous to the sequence

(SEQ ID NO: 517)

EAPGTIEFVADPAALATILSGEGVKSCHLGRQPSLAKRVLVRGSQGGTTQ
RVQGVRASAYLAPRTPTHRLDPARASCFSRLEGPGPRGRTLCPQRLQALI
SPSGPSFHPSTRPSFQELRRETAGSSRTSVSQASGLLLETPVQPAFSLPK
GEREVVTHSDEGGVASLGLAQRVPLRENREMSHTRDSHDSHLMPSPAPVA
QPLPGHVVPGPSPFGRAQRVPSPGPPTLTSYSVLRRLTVQPKTRFTPMPS
TPRVQQAQWLRGVSPQSCSEDPALPWEQVAVRLFDQESCIRSLEGSGKPP
VATPSGPHSNRTPSLQEVKIQRIGILQQLLRQEVEGLVGGQCVPLNGGSS
LDMVELQPLLTEISRTLNATEHNSGTSHLPGLLKHSGLPKPCLPEECGEP
QPCPPAEPGPPEAFCRSEPEIPEPSLQEQLEVPEPYPPAEPRPLESCCRS
EPEIPESSRQEQLEVPEPCPPAEPRPLESYCRIEPEIPESSRQEQLEVPE
PCPPAEPGPLQPSTQGQSGPPGPCPRVELGASEPCTLEHRSLESSLPPCC
SQWAPATTSLIFSSQHPLCASPPICSLQSLRPPAGQAGKELAGKECEHKR
SSPHCELHTCPAPTPGNLMLPHLPPWPSLALPQEEGRGCTSSPVLLIGLA
VGGGGGEDSTWWKYRTPDLPLNFPCPSGLSNLAPRTLALRERLKSCLTAI
HCFHEARLDDECAFYTSRAPPSGPTRVCTNPVATLLEWQDALCFIPVGSA
APQGSP
of

(SEQ ID NO: 506)

T86235_P7.

In some embodiments, the isolated chimeric proteins or polypeptides of the invention may comprise an amino acid sequence corresponding to or homologous to that as set forth in T86235_P11 (SEQ ID NO:473), comprising a first amino acid sequence being at least about 90% homologous to amino acids 1-223 of TAST_HUMAN (SEQ ID NO: 463), which also corresponds to amino acids 1-223 of T86235_P11 (SEQ ID NO:473), a bridging amino acid R corresponding to amino acid 224 of T86235_P11 (SEQ ID NO:473), a second amino acid sequence being at least about 90% homologous to amino acids 225-576 of TAST_HUMAN (SEQ ID NO: 463), which also corresponds to amino acids 225-576 of T86235_P11 (SEQ ID NO:473), a third amino acid sequence being at least about 90% homologous to amino acids 606-764 of TAST_HUMAN (SEQ ID NO: 463), which also corresponds to amino acids 577-735 of T86235_P11 (SEQ ID NO:473), and a fourth amino acid sequence being at least about 70%, optionally at least 80%, preferably at least 85%, more preferably at least 90% and most preferably at least 95% homologous to a polypeptide having the sequence VRLQPTAQLWLHSEPDGRWGTGTGGHLGFFTERSAGRLGYSARLAEL (SEQ ID NO: 518) corresponding to amino acids 736-782 of T86235_P11 (SEQ ID NO:473), wherein said first amino acid sequence, bridging amino acid, second amino acid sequence, third amino acid sequence and fourth amino acid sequence are contiguous and in a sequential order.

In some embodiments, the isolated chimeric proteins or polypeptides of the invention may comprise an amino acid sequence corresponding to or homologous to that as set forth in an edge portion of T86235_P11 (SEQ ID NO:473), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EE, having a structure as follows: a sequence starting from any of amino acid numbers 576−x to 576; and ending at any of amino acid numbers 577+((n−2)−x), in which x varies from 0 to n−2.

In some embodiments, this invention provides an isolated polypeptide encoding for an edge portion of T86235_P11 (SEQ ID NO:473), comprising an amino acid sequence being at least about 70%, optionally at least about 80%, preferably at least about 85%, more preferably at least about 90% and most preferably at least about 95% homologous to the sequence VRLQPTAQLWLHSEPDGRWGTGTGGHLGFFTERSAGRLGYSARLAEL (SEQ ID NO: 518) of T86235_P11 (SEQ ID NO:473).

In some embodiments, the isolated chimeric proteins or polypeptides of the invention may comprise an amino acid sequence corresponding to or homologous to that as set forth in T86235_P11 (SEQ ID NO:473), comprising a first amino acid sequence being at least about 90% homologous to amino acids 1-576 of Q8N5B2_HUMAN (SEQ ID NO: 467), which also corresponds to amino acids 1-576 of T86235_P11 (SEQ ID NO:473), a second amino acid sequence being at least about 90% homologous to amino acids 606-764 of Q8N5B2_HUMAN (SEQ ID NO: 467), which also corresponds to amino acids 577-735 of T86235_P11 (SEQ ID NO:473), and a third amino acid sequence being at least about 70%, optionally at least 80%, preferably at least 85%, more preferably at least 90% and most preferably at least 95% homologous to a polypeptide having the sequence VRLQPTAQLWLHSEPDGRWGTGTGGHLGFFTERSAGRLGYSARLAEL (SEQ ID NO: 518) corresponding to amino acids 736-782 of T86235_P11 (SEQ ID NO:473), wherein said first amino acid sequence, second amino acid sequence and third amino acid sequence are contiguous and in a sequential order.

In some embodiments, the isolated chimeric proteins or polypeptides of the invention may comprise an amino acid sequence corresponding to or homologous to that as set forth in an edge portion of T86235P11 (SEQ ID NO:473), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EE, having a structure as follows: a sequence starting from any of amino acid numbers 576−x to 576; and ending at any of amino acid numbers 577+((n−2)−x), in which x varies from 0 to n−2.

In some embodiments, this invention provides an isolated polypeptide encoding for an edge portion of T86235_P111 (SEQ ID NO:473), comprising an amino acid sequence being at least about 70%, optionally at least about 80%, preferably at least about 85%, more preferably at least about 90% and most preferably at least about 95% homologous to the sequence VRLQPTAQLWLHSEPDGRWGTGTGGHLGFFTERSAGRLGYSARLAEL (SEQ ID NO: 518) of T86235_P111 (SEQ ID NO:473).

In some embodiments, the isolated chimeric proteins or polypeptides of the invention may comprise an amino acid sequence corresponding to or homologous to that as set forth in T86235_P11 (SEQ ID NO:473), comprising a first amino acid sequence being at least about 90% homologous to amino acids 1-112 of Q6PJU7_HUMAN (SEQ ID NO: 439), which also corresponds to amino acids 1-112 of T86235_P11 (SEQ ID NO:473), and a second amino acid sequence being at least about 70%, optionally at least 80%, preferably at least 85%, more preferably at least 90% and most preferably at least 95% homologous to a polypeptide having the sequence EAPGTIEFVADPAALATILSGEGVKSCHLGRQPSLAKRVLVRGSQGGTTQRVQGVRASAYLAPRTPTHRLD PARASCFSRLEGPGPRGRTLCPQRLQALISPSGPSFHPSTRPSFQELRRETAGSSRTSVSQASGLLLETPVQPA FSLPKGEREVVTHSDEGGVASLGLAQRVPLRENREMSHTRDSHDSHLMPSPAPVAQPLPGHVVPCPSPFGR AQRVPSPGPPTLTSYSVLRRLTVQPKTRFTPMPSTPRVQQAQWLRGVSPQSCSEDPALPWEQVAVRLFDQE SCIRSLEGSGKPPVATPSGPHSNRTPSLQEVKIQRIGILQQLLRQEVEGLVGGQCVPLNGGSSLDMVELQPL LTEISRTLNATEHNSGTSHLPGLLKHSGLPKPCLPEECGEPQPCPPAEPGPPEAFCRSEPEIPEPSLQEQLEVP EPYPPAEPRPLESCCRSEPEIPESSRQEQLEEQLEVPEPCPPAEPGPLQPSTQGQSGPPGPCPRVELGASEPCT LEHRSLESSLPPCCSQWAPATTSLIFSSQHPLCASPPICSLQSLRPPAGQAGLSNLAPRTLALRERLKSCLTAI HCFHEARLDDECAFYTSRAPPSGPTRVCTNPVATLLEWQDALVRLQPTAQLWLHSEPDGRWGTGTOGHL GFFTERSAGRLGYSARLAEL (SEQ ID NO: 519) corresponding to amino acids 113-782 of T86235_P11 (SEQ ID NO:473), wherein said first amino acid sequence and second amino acid sequence are contiguous and in a sequential order.

In some embodiments, this invention provides an isolated polypeptide encoding for an edge portion of T86235_P11 (SEQ ID NO:473), comprising an amino acid sequence being at least about 70%, optionally at least about 80%, preferably at least about 85%, more preferably at least about 90% and most preferably at least about 95% homologous to the sequence

(SEQ ID NO: 519)

EAPGTIEFVADPAALATILSGEGVKSCHLGRQPSLAKRVLVRGSQGGTTQ
RVQGVRASAYLAPRTPTHRLDPARASCFSRLEGPGPRGRTLCPQRLQALI
SPSGPSFHPSTRPSFQELRRETAGSSRTSVSQASGLLLETPVQPAFSLPK
GEREVVTHSDEGGVASLGLAQRVPLRENREMSHTRDSHDSHLMPSPAPVA
QPLPGHVVPCPSPFGRAQRVPSPGPPTLTSYSVLRRLTVQPKTRFTPMPS
TPRVQQAQWLRGVSPQSCSEDPALPWEQVAVRLFDQESCIRSLEGSGKPP
VATPSGPHSNRTPSLQEVKAQRIGILQQLLRQEVEGLVGGQCVPLNGGSS
LDMVELQPLLTEISRTLNATEHNSGTSHLPGLLKHSGLPKPCLPEECGEP
QPCPPAEPGPPEAFCRSEPEIPEPSLQEQLEVPEPYPPAEPRPLESCCRS
EPEIPESSRQEQLEEQLEVPEPCPPAEPGPLQPSTQGQSGPPGPCPRVEL
GASEPCTLEHRSLESSLPPCCSQWAPATTSLIFSSQHPLCASPPICSLQS
LRPPAGQAGLSNLAPRTLALRERLKSCLTAIHCFHEARLDDECAFYTSRA
PPSGPTRVCTNPVATLLEWQDALVRIQPTAQLWLHSEPDGRWGTGTGGHL
GFFTERSAGRLGYSARLAEL
of

(SEQ ID NO: 473)

T86235_P11.

In some embodiments, such isolated chimeric proteins or polypeptides may comprise an amino acid sequence corresponding to or homologous to that set forth in T86235_P15 (SEQ ID NO:474).

In some embodiments, the isolated chimeric proteins or polypeptides of the invention may comprise an amino acid sequence corresponding to or homologous to that as set forth in T86235_P15 (SEQ ID NO:474), comprising a first amino acid sequence being at least about 90% homologous to amino acids 1-223 of TAST_HUMAN (SEQ ID NO: 463), which also corresponds to amino acids 1-223 of T86235_P15 (SEQ ID NO:474), a bridging amino acid R corresponding to amino acid 224 of T86235_P15 (SEQ ID NO:474), a second amino acid sequence being at least about 90% homologous to amino acids 225-576 of TAST_HUMAN (SEQ ID NO: 463), which also corresponds to amino acids 225-576 of T86235_P15 (SEQ ID NO:474), a third amino acid sequence being at least about 90% homologous to amino acids 606-699 of TAST_HUMAN (SEQ ID NO: 463), which also corresponds to amino acids 577-670 of T86235_P15 (SEQ ID NO:474), a fourth amino acid sequence being at least about 70%, optionally at least 80%, preferably at least 85%, more preferably at least 90% and most preferably at least 95% homologous to a polypeptide having the sequence GKELAGKECEHKRSSPHCELHTCPAPTPGNLMLPHLPPWPSLALPQEEGRGCTSSPVLLIGLAVGGGGGED STWWKYRTPDLPLNFPCPS (SEQ ID NO: 514) corresponding to amino acids 671-760 of T86235_P15 (SEQ ID NO:474), a fifth amino acid sequence being at least about 90% homologous to amino acids 700-764 of TAST_HUMAN (SEQ ID NO: 463), which also corresponds to amino acids 761-825 of T86235_P15 (SEQ ID NO:474), and a sixth amino acid sequence being at least about 70%, optionally at least 80%, preferably at least 85%, more preferably at least 90% and most preferably at least 95% homologous to a polypeptide having the sequence VRLQPTAQLWLHSEPDGRWGTGTGGHLGFFTERSAGRLGYSARLAEL (SEQ ID NO: 518) corresponding to amino acids 826-872 of T86235_P15 (SEQ ID NO:474), wherein said first amino acid sequence, bridging amino acid, second amino acid sequence, third amino acid sequence, fourth amino acid sequence, fifth amino acid sequence and sixth amino acid sequence are contiguous and in a sequential order.

In some embodiments, the isolated chimeric proteins or polypeptides of the invention may comprise an amino acid sequence corresponding to or homologous to that as set forth in an edge portion of T86235_P15 (SEQ ID NO:474), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EE, having a structure as follows: a sequence starting from any of amino acid numbers 576−x to 576; and ending at any of amino acid numbers 577+((n−2)−x), in which x varies from 0 to n−2.

In some embodiments, this invention provides an isolated polypeptide encoding for an edge portion of T86235_P1_{—}5 (SEQ ID NO:474), comprising an amino acid sequence being at least about 70%, optionally at least about 80%, preferably at least about 85%, more preferably at least about 90% and most preferably at least about 95% homologous to the sequence

(SEQ ID NO: 514)

GKELAGKECEHKRSSPHCELHTCPAPTPGNLMLPHLPPWPSLALPQEEGR
GCTSSPVLLIGLAVGGGGGEDSTWWKYRTPDLPLNFPCPS
of

(SEQ ID NO: 474)

T86235_P15.

In some embodiments, this invention provides an isolated polypeptide encoding for an edge portion of T86235_P15 (SEQ ID NO:474), comprising an amino acid sequence being at least about 70%, optionally at least about 80%, preferably at least about 85%, more preferably at least about 90% and most preferably at least about 95% homologous to the sequence VRLQPTAQLWLHSEPDGRWGTGTGGHLGFFTERSAGRLGYSARLAEL (SEQ ID NO: 518) of T86235_P15 (SEQ ID NO:474).

In some embodiments, the isolated chimeric proteins or polypeptides of the invention may comprise an amino acid sequence corresponding to or homologous to that as set forth in T86235_P15 (SEQ ID NO:474), comprising a first amino acid sequence being at least about 90% homologous to amino acids 1-576 of Q8N5B2_HUMAN (SEQ ID NO: 467), which also corresponds to amino acids 1-576 of T86235_P15 (SEQ ID NO:474), a second amino acid sequence being at least about 90% homologous to amino acids 606-699 of Q8N5B2_HUMAN (SEQ ID NO: 467), which also corresponds to amino acids 577-670 of T86235_P15 (SEQ ID NO:474), a third amino acid sequence being at least about 70%, optionally at least 80%, preferably at least 85%, more preferably at least 90% and most preferably at least 95% homologous to a polypeptide having the sequence GKELAGKECEHKRSSPHCELHTCPAPTPGNLMLPHLPPWPSLALPQEEGRGCTSSPVLLIGLAVGGGGGED STWWKYRTPDLPLNFPCPS (SEQ ID NO: 514) corresponding to amino acids 671-760 of T86235_P15 (SEQ ID NO:474), a fourth amino acid sequence being at least about 90% homologous to amino acids 700-764 of Q8N5B2_HUMAN (SEQ ID NO: 467), which also corresponds to amino acids 761-825 of T86235_P15 (SEQ ID NO:474), and a fifth amino acid sequence being at least about 70%, optionally at least 80%, preferably at least 85%, more preferably at least 90% and most preferably at least 95% homologous to a polypeptide having the sequence VRLQPTAQLWLHSEPDGRWGTGTGGHLGFFTERSAGRLGYSARLAEL (SEQ ID NO: 518) corresponding to amino acids 826-872 of T86235_P15 (SEQ ID NO:474), wherein said first amino acid sequence, second amino acid sequence, third amino acid sequence, fourth amino acid sequence and fifth amino acid sequence are contiguous and in a sequential order.

In some embodiments, the isolated chimeric proteins or polypeptides of the invention may comprise an amino acid sequence corresponding to or homologous to that as set forth in T86235_P15 (SEQ ID NO:474), comprising a first amino acid sequence being at least about 90% homologous to amino acids 1-112 of Q6PJU7_HUMAN (SEQ ID NO: 439), which also corresponds to amino acids 1-112 of T86235_P15 (SEQ ID NO:474), and a second amino acid sequence being at least about 70%, optionally at least 80%, preferably at least 85%, more preferably at least 90% and most preferably at least 95% homologous to a polypeptide having the sequence EAPGTIEFVADPAALATILSGEGVKSCHLGRQPSLAKRVLVRGSQGGTTQRVQGVRASAYLAPRTPTHRLD PARASCFSRLEGPGPRGRTLCPQRLQALISPSGPSFHPSTRPSFQELRRETAGSSRTSVSQASGLLLETPVQPA FSLPKGEREVVTHSDEGGVASLGLAQRVPLRENREMSHTRDSHDSHLMPSPAPVAQPLPGHVVPCPSPFGR AQRVPSPGPPTLTSYSVLRRLTVQPKTRFTPMPSTPRVQQAQWLRGVSPQSCSEDPALPWEQVAVRLFDQE SCIRSLEGSGKPPVATPSGPHSNRTPSLQEVKIQRIGILQQLLRQEVEGLVGGQCVPLNGGSSLDMVELQPL LTEISRTLNATEHNSGTSHLPGLLKHSGLPKPCLPEECGEPQPCPPAEPGPPEAFCRSEPEIPEPSLQEQLEVP EPYPPAEPRPLESCCRSEPEIPESSRQEQLEEQLEVPEPCPPAEPGPLQPSTQGQSGPPGPCPRVELGASEPCT LEHRSLESSLPPCCSQWAPATTSLIFSSQHPLCASPPICSLQSLRPPAGQAGKELAGKECEHKRSSPHCELHT CPAPTPGNLMLPHLPPWPSLALPQEEGRGCTSSPVLLIGLAVGGGGGEDSTWWKYRTPDLPLNFPCPSGLS NLAPRTLALRERLKSCLTAIHCFHEARLDDECAFYTSRAPPSGPTIRVCTNPVATLLEWQDALVRLQPTAQL WLHSEPDGRWGTGTGGHLGFFTERSAGRLGYSARLAEL (SEQ ID NO: 522) corresponding to amino acids 113-872 of T86235_P15 (SEQ ID NO:474), wherein said first amino acid sequence and second amino acid sequence are contiguous and in a sequential order.

In some embodiments, this invention provides an isolated polypeptide encoding for an edge portion of T86235_P15 (SEQ ID NO:474), comprising an amino acid sequence being at least about 70%, optionally at least about 80%, preferably at least about 85%, more preferably at least about 90% and most preferably at least about 95% homologous to the sequence

(SEQ ID NO: 522)

EAPGTIEFVADPAALATILSGEGVKSCTLGRQPSLAKRVLVRGSQGGTTQ
RVQGVRASAYLAPRTPTHRLDPARASCFSRLEGPGPRGRTLCPQRLQALI
SPSGPSFHPSTRPSFQELRRETAGSSRTSVSQASGLLLETPVQPAFSLPK
GEREVVTHSDEGGVASLGLAQRVPLRENREMSHTRDSHDSHLMPSPAPVA
QPLPGHVVPCPSPFGRAQRVPSPGPPTLTSYSVLRRLTVQPKTRFTPMPS
TPRVQQAQWLRGVSPQSCSEDPALPWEQVAVRLFDQESCIRSLEGSGKPP
VATPSGPHSNRTPSLQEVKIQRIGILQQLLRQEVEGLVGGQGVPLNGGSS
LDMVELQPLLTEISRTLNATEHNSGTSHLPGLLKHSGLPKPCLPEECGEP
QPCPPAEPGPPEAFCRSEPEIPEPSLQEQLEVPEPYPPAEPRPLESCCRS
EPEIPESSRQEQLEEQLEVPEPCPPAEPGPLQPSTQGQSGPPGPCPRVEL
GASEPCTLEHRSLESSLPPCCSQWAPATTSLIFSSQHPLCASPPICSLQS
LRPPAGQAGKELAGKECEHKRSSPHCELHTCPAPTPGNLMLPHLPPWPSL
ALPQEEGRGCTSSPVLLIGLAVGGGGGEDSTWWKYRTPDLPLNFPCPSGL
SNLAPRTLALRERLKSCLTAIHCFHEARLDDECAFYTSRAPPSGPTRVCT
NPVATLLEWQDALVRLQPTAQLWLHSEPDGRWGTGTGGHLGFFTERSAGR
LGYSARLAEL
of

(SEQ ID NO: 474)

T86235_P15.

In some embodiments, such isolated chimeric proteins or polypeptides may comprise an amino acid sequence corresponding to or homologous to that set forth in T86235_P21 (SEQ ID NO:475).

In some embodiments, the isolated chimeric proteins or polypeptides of the invention may comprise an amino acid sequence corresponding to or homologous to that as set forth in T86235_P21 (SEQ ID NO:475), comprising a first amino acid sequence being at least about 70%, optionally at least 80%, preferably at least 85%, more preferably at least 90% and most preferably at least 95%, homologous to a polypeptide having the sequence KKKKKISQIQLNESLAHLVPQSSYARGPIPLSLPPSFSFSSFLA (SEQ ID NO: 523) corresponding to amino acids 1-44 of T86235_P21 (SEQ ID NO:475), a second amino acid sequence being at least about 90% homologous to amino acids 113-223 of TAST_HUMAN (SEQ ID NO: 463), which also corresponds to amino acids 45-155 of T86235_P21 (SEQ ID NO:475), a bridging amino acid R corresponding to amino acid 156 of T86235_P21 (SEQ ID NO:475), a third amino acid sequence being at least about 90% homologous to amino acids 225-576 of TAST_HUMAN (SEQ ID NO: 463), which also corresponds to amino acids 157-508 of T86235_P21 (SEQ ID NO:475), and a fourth amino acid sequence being at least about 90% homologous to amino acids 606-778 of TAST_HUMAN (SEQ ID NO: 463), which also corresponds to amino acids 509-681 of T86235_P21 (SEQ ID NO:475), wherein said first amino acid sequence, second amino acid sequence, bridging amino acid, third amino acid sequence and fourth amino acid sequence are contiguous and in a sequential order.

In some embodiments, this invention provides an isolated polypeptide encoding for a head of T86235_P21 (SEQ ID NO:475), comprising a polypeptide being at least 70%, optionally at least about 80%, preferably at least about 85%, more preferably at least about 90% and most preferably at least about 95% homologous to the sequence KKKKKISQIQLNESLAHLVPQSSYARGPIPLSLPPSFSFSSFLA (SEQ ID NO: 523) of T86235_P21 (SEQ ID NO:475).

In some embodiments, the isolated chimeric proteins or polypeptides of the invention may comprise an amino acid sequence corresponding to or homologous to that as set forth in an edge portion of T86235_P21 (SEQ ID NO:475), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EE, having a structure as follows: a sequence starting from any of amino acid numbers 508−x to 508; and ending at any of amino acid numbers 509+((n−2)−x), in which x varies from 0 to n−2.

In some embodiments, the isolated chimeric proteins or polypeptides of the invention may comprise an amino acid sequence corresponding to or homologous to that as set forth in T86235_P21 (SEQ ID NO:475), comprising a first amino acid sequence being at least about 70%, optionally at least 80%, preferably at least 85%, more preferably at least 90% and most preferably at least 95%, homologous to a polypeptide having the sequence KKKKKISQIQLNESLAHLVPQSSYARGPIPLSLPPSFSFSSFLA (SEQ ID NO: 523) corresponding to amino acids 1-44 of T86235_P21 (SEQ ID NO:475), a second amino acid sequence being at least about 90% homologous to amino acids 113-576 of Q8N5B2_HUMAN (SEQ ID NO: 467), which also corresponds to amino acids 45-508 of T86235_P21 (SEQ ID NO:475), and a third amino acid sequence being at least about 90% homologous to amino acids 606-778 of Q8N5B2_HUMAN (SEQ ID NO: 467), which also corresponds to amino acids 509-681 of T86235_P21 (SEQ ID NO:475), wherein said first amino acid sequence, second amino acid sequence and third amino acid sequence are contiguous and in a sequential order.

In some embodiments, the isolated chimeric proteins or polypeptides of the invention may comprise an amino acid sequence corresponding to or homologous to that as set forth in T86235_P26 (SEQ ID NO:476), comprising a first amino acid sequence being at least about 90% homologous to amino acids 465-576 of TAST_HUMAN (SEQ ID NO: 463), which also corresponds to amino acids 1-112 of T86235_P26 (SEQ ID NO:476), and a second amino acid sequence being at least about 90% homologous to amino acids 606-778 of TAST_HUMAN (SEQ ID NO: 463), which also corresponds to amino acids 113-285 of T86235_P26 (SEQ ID NO:476), wherein said, first amino acid sequence and second amino acid sequence are contiguous and in a sequential order.

In some embodiments, the isolated chimeric proteins or polypeptides of the invention may comprise an amino acid sequence corresponding to or homologous to that as set forth in an edge portion of T86235_P26 (SEQ ID NO:476), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EE, having a structure as follows: a sequence starting from any of amino acid numbers 112−x to 112; and ending at any of amino acid numbers 113+((n−2)−x), in which x varies from 0 to n−2.

In some embodiments, the isolated chimeric proteins or polypeptides of the invention may comprise an amino acid sequence corresponding to or homologous to that as set forth in T86235_P26 (SEQ ID NO:476), comprising a first amino acid sequence being at least about 90% homologous to amino acids 465-576 of Q8N5B2_HUMAN (SEQ ID NO: 467), which also corresponds to amino acids 1-112 of T86235_P26 (SEQ ID NO:476), and a second amino acid sequence being at least about 90% homologous to amino acids 606-778 of Q8N5B2_HUMAN (SEQ ID NO: 467), which also corresponds to amino acids 113-285 of T86235_P26 (SEQ ID NO:476), wherein said, first amino acid sequence and second amino acid sequence are contiguous and in a sequential order.

In some embodiments, the term “polypeptide” is to be understood to refer to a molecule comprising from at least 2 to several thousand or more amino acids. The term “polypeptide” is to be understood to include, inter alia, native peptides (either degradation products, synthetically synthesized peptides or recombinant peptides), peptidomimetics, such as peptoids and semipeptoids or peptide analogs, which may comprise, for example, any desirable modification, including, inter alia, modifications rendering the peptides more stable while in a body or more capable of penetrating into cells, or others as will be appreciated by one skilled in the art. Such modifications include, but are not limited to N terminus modification, C terminus modification, peptide bond modification, backbone modifications, residue modification, or others. Inclusion of such peptides within the polypeptides of this invention may produce a polypeptide sharing identity with the polypeptides described herein, for example, those provided in the sequence listing.

Methods for preparing, isolating, deriving, etc., the polypeptides of this invention are well known in the art. In some embodiments, the polypeptides of this invention comprise variants of known proteins. For example, and in some embodiments, the polypeptides of this invention comprise splice variants of native proteins expressed in a given subject. In some embodiments, the polypeptides may be obtained through known protein evolution techniques available in the art. In some embodiments, the polypeptides of this invention may be obtained via rational design, based on a particular native polypeptide sequence.

In some embodiments, this invention provides for antibodies or antibody fragments specifically interacting with or recognizing a polypeptide of this invention.

In one embodiment, the antibody recognizes one or more epitopes (antigen determinants) contained within the polypeptides of this invention. In some embodiments, reference to the antibody property of “specific interaction” or “recognition” is to be understood as including covalent and/or non-covalent associations, and with a variance of affinity over several orders of magnitude. Such terms are to be understood as relative, with respect to an index molecule, for which the antibody is though to have little to no specific interaction or recognition.

In one embodiment, the antibodies will specifically interact or recognize a particular antigen determinant. In some embodiments, the antibodies or antibody fragments of this invention will recognize or interact with a polypeptide or protein of the invention, and will not substantially recognize or interact with other molecules, even when present in the same sample, such as a biological sample. In some embodiments, the antibodies of this invention have a specificity such that the specific interaction with or binding to the antigen is at least about 2, or in some embodiments, at least about 5, or in some embodiments, at least about 10-fold greater than interaction or binding observed under the same reaction conditions with a molecule that does not include the antigenic determinant.

The antibodies may be useful, in some embodiments, in detecting qualitative and/or quantitative changes in expression of the polypeptides or polynucleotides of this invention. In some embodiments, changes in expression are associated with a particular disease or disorder, such that detection of such changes comprises a diagnostic method of this invention.

In one embodiment, this invention provides a diagnostic kit for detecting a disease, comprising reagents which detect qualitative and/or quantitative changes in expression of a polypeptide or polynucleotide of this invention.

Optionally, the kit comprises a NAT-based technology; optionally and preferably, the kit further comprises at least one nucleotide probe or primer, alternatively and optionally this kit comprises at least one primer pair capable of selectively hybridizing to a nucleic acid sequence as described herein; alternatively and optionally, said kit further comprises at least one oligonucleotide capable of selectively hybridizing to a nucleic acid sequence according to any of the above claims.

Alternatively and optionally, the kit comprises an antibody according to any of the above claims, optionally and preferably, the kit further comprises at least one reagent for performing an ELISA or a Western blot.

In some embodiments, this invention provides a diagnostic method, for example, a method of detection of a polypeptide or polynucleotide of this invention, whereby expression, or relative changes in expression of the polypeptide or polynucleotide herald the onset, severity, or prognosis of an individual with regard to a particular disease, disorder or condition.

In some embodiments, such detection may comprise detection of specific expression of a splice variant, or other polypeptide or polynucleotide of this invention, via any means known in the art, and as described herein.

In some embodiments, detection of the following genes and/or any expressed transcripts, and/or proteins, preferably including any splice variants, forms part of the diagnostic methods of this invention: AA383349, H61775, AA583399, H13410, T86235HUMPAX8A, N31842, T58132, or any combination thereof.

In some embodiments, detection of AA383349_T6 (SEQ ID NO: 334), AA383349_T7 (SEQ ID NO: 1), AA383349_T16 (SEQ ID NO: 2), AA383349_T17 (SEQ ID NO: 3), AA383349_P4 (SEQ ID NO: 33), AA383349_P5 (SEQ ID NO: 34), AA383349_P11 (SEQ ID NO: 35), AA383349_P12 (SEQ ID NO: 36), H61775_T22 (SEQ ID NO:345), H61775T23 (SEQ ID NO:346), H61775_P17 (SEQ ID NO:354), H61775_P18 (SEQ ID NO:355), H13410_T8 (SEQ ID NO: 87), H13410_T12 (SEQ ID NO: 88), H13410_T18 (SEQ ID NO: 89), H13410_T21 (SEQ ID NO: 90), H13410_T27 (SEQ ID NO: 91), H13410_T35 (SEQ ID NO: 92), H13410_P4 (SEQ ID NO: 139), H13410_P8 (SEQ ID NO: 140), H13410_P9 (SEQ ID NO: 141), H13410_P20 (SEQ ID NO: 142), H13410_P46 (SEQ ID NO: 143), H13410_P47 (SEQ ID NO: 144), and known H13410 amino acid (SEQ ID NOs:133-138) and/or nucleic acid sequences (SEQ ID NOs:420, 491), T86235_T5 (SEQ ID NO:440), T86235_T6 (SEQ ID NO:441), T86235_T7 (SEQ ID NO:442), T86235_T11 (SEQ ID NO:443), T86235_T13 (SEQ ID NO:444), T86235_T15 (SEQ ID NO:445), T86235_T18 (SEQ ID NO:446), T86235_T26 (SEQ ID NO:447), T86235_T32 (SEQ ID NO:448), T86235_T36 (SEQ ID NO:449), T86235_P4 (SEQ ID NO:469), T86235_P5 (SEQ ID NO:470), T86235_P6 (SEQ ID NO:471), T86235_P7 (SEQ ID NO:472), T86235_P1 (SEQ ID NO:473), T86235_P15 (SEQ ID NO:474), T86235_P21 (SEQ ID NO:475), T86235_P26 (SEQ ID NO:476), T86235_P30 (SEQ ID NO:477) and WT, N31842_T4 (SEQ ID NO:478), N31842_T5 (SEQ ID NO:479), N31842_T6 (SEQ ID NO:480), N31842_P3 (SEQ ID NO:488), N31842_P4 (SEQ ID NO:489), N31842_P5 (SEQ ID NO:490), or relative changes in expression and/or level of these variants is indicative of the presence, onset, severity or prognosis, and/or staging, and/or progression of breast cancer in a subject. In some embodiments, the polypeptides, polynucleotides and/or methods of this invention may be useful in the treatment selection and monitoring, diagnosis or prognosis assessment of breast cancer.

In some embodiments, detection of AA583399_T0 (SEQ ID NO: 37), AA583399_T1 (SEQ ID NO: 38), AA583399_T2 (SEQ ID NO: 39), AA583399_T3 (SEQ ID NO: 40), AA583399_T4 (SEQ ID NO: 41), AA583399_T5 (SEQ ID NO: 42), AA583399_T6 (SEQ ID NO: 43), AA583399_T7 (SEQ ID NO: 44), AA583399_T8 (SEQ ID NO: 45), AA583399_T9 (SEQ ID NO: 46), AA583399_T10 (SEQ ID NO: 47), AA583399_T11 (SEQ ID NO: 48), AA583399_T12 (SEQ ID NO: 49), AA583399_T15 (SEQ ID NO: 50), AA583399_T16 (SEQ ID NO: 51), AA583399_T17 (SEQ ID NO: 52), AA583399_P3 (SEQ ID NO: 77), AA583399_P2 (SEQ ID NO: 78), AA583399_P4 (SEQ ID NO: 79), AA583399_P5 (SEQ ID NO: 80), AA583399_P6 (SEQ ID NO: 81), AA583399_P8 (SEQ ID NO: 82), AA583399_P10 (SEQ ID NO: 83), AA583399_P11 (SEQ ID NO: 84), AA583399_P12 (SEQ ID NO: 85), AA583399_P14 (SEQ ID NO: 86), and known AA583399 amino acid (SEQ ID NOs:73-76) and/or nucleic acid sequences (SEQ ID NO:433), H13410_T8 (SEQ ID NO: 87), H13410_T12 (SEQ ID NO: 88), H13410_T18 (SEQ ID NO: 89), H13410_T21 (SEQ ID NO: 90), H13410_T27 (SEQ ID NO: 91), H13410_T35 (SEQ ID NO: 92), H13410_P4 (SEQ ID NO: 139), H13410_P8 (SEQ ID NO: 140), H13410_P9 (SEQ ID NO: 141), H13410_P20 (SEQ ID NO: 142), H13410_P46 (SEQ ID NO: 143), H13410_P47 (SEQ ID NO: 144), and known H13410 amino acid (SEQ ID NOs:133-138) and/or nucleic acid sequences (SEQ ID NOs:420, 491), T86235_T5 (SEQ ID NO:440), T86235_T6 (SEQ ID NO:441), T86235_T7 (SEQ ID NO:442), T86235T111 (SEQ ID NO:443), T86235_T13 (SEQ ID NO:444), T86235_T15 (SEQ ID NO:445), T86235_T18 (SEQ ID NO:446), T86235_T26 (SEQ ID NO:447), T86235_T32 (SEQ ID NO:448), T86235_T36 (SEQ ID NO:449), T86235_P4 (SEQ ID NO:469), T86235_P5 (SEQ ID NO:470), T86235_P6 (SEQ ID NO:471), T86235_P7 (SEQ ID NO:472), T86235_P11 (SEQ ID NO:473), T86235_P15 (SEQ ID NO:474), T86235_P21 (SEQ ID NO:475), T86235_P26 (SEQ ID NO:476), T86235_P30 (SEQ ID NO:477), and WT, H61775_P18 (SEQ ID NO:355), HUMPAX8A_T21 (SEQ ID NO: 421), HUMPAX8A_T24 (SEQ ID NO: 156), HUMPAX8A_T26 (SEQ ID NO: 157), HUMPAX8A_T30 (SEQ ID NO: 158), HUMPAX8A_P33 (SEQ ID NO: 183), HUMPAX8A_P36 (SEQ ID NO: 184), HUMPAX8A_P37 (SEQ ID NO: 185), HUMPAX8A_P40 (SEQ ID NO: 186), N31842_T4 (SEQ ID NO:478), N31842_T5 (SEQ ID NO:479), N31842_T6 (SEQ ID NO:480), N31842_P3 (SEQ ID NO:488), N31842_P4 (SEQ ID NO:489), N31842_P5 (SEQ ID NO:490), T58132_T2 (SEQ ID NO: 187), T58132_T4 (SEQ ID NO: 188), T58132_T7 (SEQ ID NO: 189), T58132_T9 (SEQ ID NO: 190), T58132_T10 (SEQ ID NO: 191), T58132_T11 (SEQ ID NO: 192), T58132_T12 (SEQ ID NO: 193), T58132_P0 (SEQ ID NO: 213), T58132_P1 (SEQ ID NO: 214), T58132_P2 (SEQ ID NO: 215), T58132_P12 (SEQ ID NO: 216), T58132_P13 (SEQ ID NO: 217), or relative changes in expression and/or level of these variants, their products or certain variants thereof is indicative of the presence, onset, severity or prognosis, and/or staging, and/or progression of ovarian cancer in a subject. In some embodiments, the polypeptides, polynucleotides and/or methods of this invention may be useful in the treatment selection and monitoring, diagnosis or prognosis assessment of ovarian cancer.

In some embodiments, detection of T58132_T2 (SEQ ID NO: 187), T58132_T4 (SEQ ID NO: 188), T58132_T7 (SEQ ID NO: 189), T58132_T9 (SEQ ID NO: 190), T58132_T10 (SEQ ID NO: 191), T58132_T11 (SEQ ID NO: 192), T58132_T12 (SEQ ID NO: 193), T58132_P0 (SEQ ID NO: 213), T58132_P1 (SEQ ID NO: 214), T58132_P2 (SEQ ID NO: 215), T58132_P12 (SEQ ID NO: 216), T58132_P13 (SEQ ID NO: 217), or relative changes in expression and/or level of these variants, their products or certain variants thereof is indicative of the presence, onset, severity or prognosis, and/or staging, and/or progression of colon cancer in a subject. In some embodiments, the polypeptides, polynucleotides and/or methods of this invention may be useful in the treatment selection and monitoring, diagnosis or prognosis assessment of colon cancer.

In some embodiments, detection of AA583399_T0 (SEQ ID NO: 37), AA583399_T1 (SEQ ID NO: 38), AA583399_T2 (SEQ ID NO: 39), AA583399_T3 (SEQ ID NO: 40), AA583399_T4 (SEQ ID NO: 41), AA583399_T5 (SEQ ID NO: 42), AA583399_T6 (SEQ ID NO: 43), AA583399_T7 (SEQ ID NO: 44), AA583399_T8 (SEQ ID NO: 45), AA583399_T9 (SEQ ID NO: 46), AA583399T10 (SEQ ID NO: 47), AA583399_T11 (SEQ ID NO: 48), AA583399_T12 (SEQ ID NO: 49), AA583399_T15 (SEQ ID NO: 50), AA583399_T16 (SEQ ID NO: 51), AA583399_T17 (SEQ ID NO: 52), AA583399_P3 (SEQ ID NO: 77), AA583399_P2 (SEQ ID NO: 78), AA583399_P4 (SEQ ID NO: 79), AA583399_P5 (SEQ ID NO: 80), AA583399_P6 (SEQ ID NO: 81), AA583399_P8 (SEQ ID NO: 82), AA583399_P10 (SEQ ID NO: 83), AA583399_P11 (SEQ ID NO: 84), AA583399_P12 (SEQ ID NO: 85), AA583399_P14 (SEQ ID NO: 86), and known AA583399 amino acid (SEQ ID NOs:73-76) and/or nucleic acid sequences (SEQ ID NO:433), H13410_T8 (SEQ ID NO: 87), H13410_T12 (SEQ ID NO: 88), H13410_T18 (SEQ ID NO: 89), H13410_T21 (SEQ ID NO: 90), H13410_T27 (SEQ ID NO: 91), H13410_T35 (SEQ ID NO: 92), H13410_P4 (SEQ ID NO: 139), H13410_P8 (SEQ ID NO: 140), H13410_P9 (SEQ ID NO: 141), H13410_P20 (SEQ ID NO: 142), H13410_P46 (SEQ ID NO: 143), H13410_P47 (SEQ ID NO: 144), and known H13410 amino acid (SEQ ID NOs:133-138) and/or nucleic acid sequences (SEQ ID NOs:420, 491), T86235_T5 (SEQ ID NO:440), T86235_T6 (SEQ ID NO:441), T86235_T7 (SEQ ID NO:442), T86235_T11 (SEQ ID NO:443), T86235_T13 (SEQ ID NO:444), T86235_T15 (SEQ ID NO:445), T86235_T18 (SEQ ID NO:446), T86235_T26 (SEQ ID NO:447), T86235_T32 (SEQ ID NO:448), T86235_T36 (SEQ ID NO:449), T86235_P4 (SEQ ID NO:469), T86235_P5 (SEQ ID NO:470), T86235_P6 (SEQ ID NO:471), T86235_P7 (SEQ ID NO:472), T86235_P11 (SEQ ID NO:473), T86235_P15 (SEQ ID NO:474), T86235_P21 (SEQ ID NO:475), T86235_P26 (SEQ ID NO:476), and known T86235 amino acid (SEQ ID NOs:437, 438, 462-468) and/or nucleic acid sequences (SEQ ID NOs:436, 438), H61775_P18 (SEQ ID NO:355), N31842_P3 (SEQ ID NO:488), T58132_T2 (SEQ ID NO: 187), T58132_T4 (SEQ ID NO: 188), T58132_T7 (SEQ ID NO: 189), T58132_T9 (SEQ ID NO: 190), T58132_T10 (SEQ ID NO: 191), T58132_T11 (SEQ ID NO: 192), T58132_T12 (SEQ ID NO: 193), T58132_P0 (SEQ ID NO: 213), T58132_P1 (SEQ ID NO: 214), T58132_P2 (SEQ ID NO: 215), T58132_P12 (SEQ ID NO: 216), T58132_P13 (SEQ ID NO: 217), or relative changes in expression and/or level of these variants, their products or certain variants thereof is indicative of the presence, onset, severity or prognosis, and/or staging, and/or progression of lung cancer in a subject. In some embodiments, the polypeptides, polynucleotides and/or methods of this invention may be useful in the treatment selection and monitoring, diagnosis or prognosis assessment of lung cancer. In some embodiments, detection of N31842_T4 (SEQ ID NO:478), N31842_T5 (SEQ ID NO:479), N31842_T6 (SEQ ID NO:480), N31842_P3 (SEQ ID NO:488), N31842_P4 (SEQ ID NO:489), N31842_P5 (SEQ ID NO:490), or relative changes in expression of these variants, their products or certain variants thereof is indicative of the presence, onset, severity or prognosis and/or staging and or progression of renal disease and/or condition including but not limited to any type of renal damage; renal cancer, including but not limited to renal cell carcinoma; polycystic kidney disease; Diabetes induced nephropathy; Chronic Kidney Disease; Total Kidney Failure; Autoimmune nephropathy, including but not limited to Systemic lupus erythematosus (SLE), Goodpasture's syndrome, IgA nephropathy; Hereditary Nephritis—(Alport Syndrome); Infection-related Glomerular Disease, including but not limited to Acute post-streptococcal glomerulonephritis (PSGN), Bacterial endocarditis, HIV; Glomerulosclerosis; Focal segmental glomenilosclerosis (FSGS); Membranous nephropathy; Minimal change disease (MCD) in a subject. In some embodiments, the polypeptides, polynucleotides and/or methods of this invention may be useful in the treatment selection and monitoring, diagnosis or prognosis assessment of any type of renal disease.

The polypeptides and/or polynucleotides of this invention may serve as markers or indicators of disease initiation, severity and/or response to treatment, for the indicated disease, disorder or condition, and their use as such is to be considered part of this invention, and part of the methods of this invention.

In some embodiments, the phrase “marker-detectable disease is a particular cluster marker detectable disease” refers to the fact that any polynucleotides and/or polypeptides of this invention can be used to detect the indicated disease, or assess the parameters of the disease, etc., as described herein. In some embodiments, a particular cluster will be useful for the diagnosis, assessment and prognostic indications regarding the indicated disease disorder or condition.

In one embodiment, the marker-detectable disease is cluster AA383349, or cluster H61775 marker-detectable disease and is a cancer, including but not limited to breast cancer, breast cancer invasion and metastasis.

In another embodiment, the marker-detectable disease is a cluster AA583399 marker-detectable disease and is a cancer, including but not limited to ovary cancer, lung cancer, ovary and lung cancer invasion and metastasis.

In another embodiment, the marker-detectable disease is cluster H13410, cluster T86235 or H61775_P18 (SEQ ID NO:355) marker-detectable disease and is a cancer, including but not limited to ovary cancer, lung cancer, breast cancer, ovary, lung and breast cancer invasion and metastasis.

In another embodiment, the marker-detectable disease is a cluster HUMPAX8A marker-detectable disease and is a cancer, including but not limited to ovary cancer, ovary cancer invasion and metastasis.

In another embodiment, the marker-detectable disease is a cluster N31842 marker-detectable disease and is a cancer, including but not limited to ovary cancer, lung cancer, breast cancer, ovary, lung and breast cancer invasion and metastasis.

In another embodiment, the marker-detectable disease is a cluster T58132 marker-detectable disease and is a cancer, including but not limited to colon cancer, ovary cancer, lung cancer, colon, ovary and lung cancer invasion and metastasis.

In another embodiment, the marker-detectable disease is a cluster N31842 marker-detectable disease and is a renal disease and/or condition including but not limited to any type of renal damage; renal cancer, including but not limited to renal cell carcinoma; polycystic kidney disease; Diabetes induced nephropathy; Chronic Kidney Disease; Total Kidney Failure; Autoimmune nephropathy, including but not limited to Systemic lupus erythematosus (SLE), Goodpasture's syndrome, IgA nephropathy; Hereditary Nephritis—(Alport Syndrome); Infection-related Glomerular Disease, including but not limited to Acute post-streptococcal glomerulonephritis (PSGN), Bacterial endocarditis, HIV; Glomerulosclerosis; Focal segmental glomerulosclerosis (FSGS); Membranous nephropathy; Minimal change disease (MCD). In another embodiment, the marker-detectable disease is a cluster T58132 marker-detectable disease and is a cancer, including but not limited ovary cancer, lung cancer, colon cancer, ovary, colon and lung cancer invasion and metastasis.

In another embodiment, the following clusters: H13410, HUMPAX8A, N31842, T58132, showing age and/or stage differential diagnostic capability, the present invention provides diagnostic methods, kits and assays for diagnosis, assessment and prognostic indications regarding the indicated disease disorder or condition, according to the age of the subject and/or stage of the condition, as described herein.

With regard to lung cancer, the disease (and/or diagnostic method to be performed) comprises, in some embodiments, one or more of invasive or metastatic lung cancer; squamous cell lung carcinoma, lung adenocarcinoma, carcinoid, small cell lung cancer or non-small cell lung cancer; detection of overexpression in lung metastasis (vs. primary tumor); detection of overexpression in lung cancer, for example non small cell lung cancer, for example adenocarcinoma, squamous cell cancer or carcinoid, or large cell carcinoma; identification of a metastasis of unknown origin which originated from a primary lung cancer; assessment of a malignant tissue residing in the lung that is from a non-lung origin, including but not limited to: osteogenic and soft tissue sarcomas; colorectal, uterine, cervix and corpus tumors; head and neck, breast, testis and salivary gland cancers; melanoma; and bladder and kidney tumors; distinguishing between different types of lung cancer, therefore potentially affect treatment choice (e.g. small cell vs. non small cell tumors); analysis of unexplained dyspnea and/or chronic cough and/or hemoptysis; differential diagnosis of the origin of a pleural effusion; diagnosis of conditions which have similar symptoms, signs and complications as lung cancer and where the differential diagnosis between them and lung cancer is of clinical importance including but not limited to: non-malignant causes of lung symptoms and signs, including but not limited to: lung lesions and infiltrates, wheeze, stridor, tracheal obstruction, esophageal compression, dysphagia, recurrent laryngeal nerve paralysis, hoarseness, phrenic nerve paralysis with elevation of the hemidiaphragm and Horner syndrome; or detecting a cause of any condition suggestive of a malignant tumor including but not limited to anorexia, cachexia, weight loss, fever, hypercalcemia, hypophosphatemia, hyponatremia, syndrome of inappropriate secretion of antidiuretic hormone, elevated ANP, elevated ACTH, hypokalemia, clubbing, neurologic-myopathic syndromes and thrombophlebitis.

In some embodiments, the polypeptides and/or polynucleotides of this invention may be useful as potential markers for lung cancer, alone or in combination with one or more alternative polynucleotides or polypeptides described herein, and/or in combination with known markers for lung cancer, including but not limited to CEA, CA15-3, Beta-2-microglobulin, CA19-9, TPA, and/or in combination with the known protein(s) for the variant marker as described herein.

With regard to breast cancer, for the detection of the disease (and/or for the diagnostic method to be performed) the polypeptides and/or polynucleotides may be useful in determining a probable outcome in breast cancer; identification of a metastasis of unknown origin which originated from a primary breast cancer tumor; assessing lymphadenopathy, and in particular axillary lymphadenopathy; distinguishing between different types of breast cancer, therefore potentially affect treatment choice (e.g. as HER-2); differentially diagnosing between a benign and malignant breast mass; as a tool in the assessment of conditions affecting breast skin (e.g. Paget's disease) and their differentiation from breast cancer; differential diagnosis of breast pain or discomfort resulting from either breast cancer or other possible conditions (e.g. mastitis, Mondors syndrome); non-breast cancer conditions which have similar symptoms, signs and complications as breast cancer and where the differential diagnosis between them and breast cancer is of clinical importance including but not limited to: abnormal mammogram and/or nipple retraction and/or nipple discharge due to causes other than breast cancer, including but not limited to benign breast masses, melanoma, trauma and technical and/or anatomical variations; determining a cause of any condition suggestive of a malignant tumor including but not limited to anorexia, cachexia, weight loss, fever, hypercalcemia, paraneoplastic syndrome; or determining a cause of lymphadenopathy, weight loss and other signs and symptoms associated with breast cancer but originate from diseases different from breast cancer including but not limited to other malignancies, infections and autoimmune diseases.

Each variant marker of the present invention described herein as potential marker for breast cancer, might optionally be used alone or in combination with one or more other variant breast cancer described herein, and/or in combination with known markers for breast cancer, including but not limited to Calcitonin, CA15-3 (Mucin1), CA27-29, TPA, a combination of CA 15-3 and CEA, CA 27.29 (monoclonal antibody directed against MUC1), Estrogen 2 (beta), HER-2 (c-erbB2), and/or in combination with the known protein(s) for the variant marker as described herein.

It is to be understood that any polynucleotide or polypeptide of this invention may be useful as a marker for a disease, disorder or condition, and such use is to be considered a part of this invention.

With regard to colon cancer, the disease (and/or diagnostic method to be performed) optionally and preferably comprises one or more of invasive or metastatic colon cancer.

Each marker of the present invention described herein as potential marker for colorectal cancer, might optionally be used alone or in combination with one or more other variant colorectal cancer described herein, and/or in combination with known markers for colorectal cancer, including but not limited to CEA, CA19-9, CA50, and/or in combination with the known protein(s) for the variant marker as described herein.

With regard to ovarian cancer, the polypeptides and/or polynucleotide may be used in the diagnosis, treatment or prognostic assessment of invasive or metastatic ovarian cancer; correlating stage and malignant potential; identification of a metastasis of unknown origin which originated from a primary ovarian cancer; differential diagnosis between benign and malignant ovarian cysts; diagnosing a cause of infertility, for example differential diagnosis of various causes thereof; detecting of one or more non-ovarian cancer conditions that may elevate serum levels of ovary related markers, including but not limited to: cancers of the endometrium, cervix, fallopian tubes, pancreas, breast, lung and colon; nonmalignant conditions such as pregnancy, endometriosis, pelvic inflammatory disease and uterine fibroids; diagnosing conditions which have similar symptoms, signs and complications as ovarian cancer and where the differential diagnosis between them and ovarian cancer is of clinical importance including but not limited to: non-malignant causes of pelvic mass, including, but not limited to: benign (functional) ovarian cyst, uterine fibroids, endometriosis, benign ovarian neoplasms and inflammatory bowel lesions; determining a cause of any condition suggestive of a malignant tumor including but not limited to anorexia, cachexia, weight loss, fever, hypercalcemia, skeletal or abdominal pain, paraneoplastic syndrome, or ascites.

In some embodiments, the polypeptides and/or polynucleotides of this invention may be used in the diagnosis, treatment or prognostic assessment of ovarian cancer, alone or in combination with one or more polypeptides and/or polynucleotides of this invention, and/or in combination with known markers for ovarian cancer, including but not limited to CEA, CA125 (Mucin 16), CA72-4TAG, CA-50, CA 54-61, CA-195 and CA 19-9 in combination with CA-125, and/or in combination with the known protein(s) associated with the indicated polypeptide or polynucleotide, as described herein.

With regard to renal disease, and/or condition, the polypeptides and/or polynucleotides of this invention may be used for the diagnosis, treatment selection and monitoring, or assessment of prognosis of any type of renal disease, and/or condition including but not limited to any type of renal damage; renal cancer, including but not limited to renal cell carcinoma; polycystic kidney disease; Diabetes induced nephropathy; Chronic Kidney Disease; Total Kidney Failure; Autoimmune nephropathy, including but not limited to Systemic lupus erythematosus (SLE), Goodpasture's syndrome, IgA nephropathy; Hereditary Nephritis (Alport Syndrome); Infection-related Glomerular Disease, including but not limited to Acute post-streptococcal glomerulonephritis (PSGN), Bacterial endocarditis, HIV; Glomerulosclerosis; Focal segmental glomerulosclerosis (FSGS); Membranous nephropathy; Minimal change disease (MCD).

In some embodiments, the polypeptides/polynucleotides of this invention may be used for the diagnosis, treatment selection and monitoring, or assessment of prognosis of any type of renal disease, and/or condition in conjunction with other screening procedures and/or other markers, including but not limited to urinary protein, creatinine or creatinine clearance, and/or markers used for the diagnosis or assessment of prognosis of renal cancer, specifically of renal cell carcinoma, including but not limited to vascular endothelial growth factor, interleukin-12, the soluble interleukin-2 receptor, intercellular adhesion molecule-1, human chorionic gonadotropin beta, insulin-like growth factor-1 receptor, Carbonic anhydrase 9 (CA 9), endostatin, Thymidine phosphorylase or combinations thereof.

Detecting specific expression may in some embodiments be performed with a NAT-based technology (optionally comprising at least one nucleotide probe or primer), and/or with an immunoassay (optionally comprising an antibody according to any of the embodiments described herein).

In some embodiments, this invention provides a method of detecting, treating and/or assessing prognosis of a disease, disorder or condition, comprising detecting polypeptides and/or polynucleotides of this invention. In some embodiments, such methods are also referred to herein as methods of screening for variant-detectable disease, whereby the detection of variant expression serves as an indicator for the disease. In some embodiments, such detection may make use of a biomarker, antibody or any method or assay as described herein.

In some embodiments, this invention provides a method for screening for a disease, comprising detecting expression of:
a. a polypeptide having an amino acid sequence as set forth in SEQ ID NOs: 33-36, 77-86, 139-144, 183-186, 213-217, 354-355, 469-477, 488-490, or a homologue or fragment thereof;
b. a polypeptide comprising a bridge, edge portion, tail, or head portion, wherein said polypeptide has an amino acid sequence as set forth in SEQ ID NOs:494-523, 531-534 or a homologue or fragment thereof;
c. a polynucleotide having a nucleic acid sequence as set forth in SEQ ID NOs: 1-3, 37-52, 87-92, 155-158, 187-193, 334, 345-346, 421, 440-449, 478-480, or a homologue or fragment thereof;
d. a polynucleotide comprising a node having a nucleic acid sequence as set forth in SEQ ID NOs: 4-28, 53-72, 93-132, 159-171, 194-204, 347-349, 450-461, 481-484;
e. an antibody capable of specifically binding to at least one epitope of a polypeptide comprising an amino acid sequence as set forth in SEQ ID NOs: 33-36, 77-86, 139-144, 183-186, 213-217, 354-355, 469-477, 488-490, 494-523, 531-534;
f. an oligonucleotide having a nucleic acid sequence as set forth in SEQ ID NOs:234, 237, 240, 243, 267, 270, 273, 276, 279, 285, 288, 292-296, 358, 361, 364, 367, 370, 385, 394, 397, 400, 403, 492, 493, 524-530;
g. a primer pair, comprising a pair of isolated oligonucleotides capable of specifically hybridizing to at least a portion of a polynucleotide having a nucleic acid sequence as set forth in SEQ ID NOs: 234, 237, 240, 243, 267, 270, 273, 276, 279, 285, 288, 358, 361, 364, 367, 370, 385, 394, 397, 400, 403; or homologous thereto;
h. a primer pair, comprising a pair of isolated oligonucleotides capable of specifically hybridizing to at least a portion of a polynucleotide having a nucleic acid sequence as set forth in SEQ ID NOs:232-233, 235-236, 238-239, 241-242, 265-266, 268-269, 271-272, 274-275, 277-278, 283-284, 286-287, 356-357, 359-360, 362-363, 365-366, 368-369, 383-384, 392-393, 395-396, 398-399, 401-402,
   whereby qualitative or quantitative differences in expression as compared to an index sample is indicator for the treatment, diagnosis or assessment of prognosis of the disease, disorder or condition.

In some embodiments, a method of this invention may make use of a polynucleotide, polypeptide, vector, antibody, biomarker, or combination thereof, as described herein, including any embodiments thereof.

In some embodiments, the methods of this invention may be conducted on a cell or tissue or body fluid sample isolated from a subject having, predisposed to, or suspected of having the disease disorder or condition. In some embodiments, the methods are directed to the monitoring of disease progression and/or treatment efficacy and/or relapse of the indicated disease, disorder or condition.

In another embodiment, this invention provides methods for the selection of a particular therapy, or optimization of a given therapy for a disease, disorder or condition, the method comprising quantitatively and/or qualitatively determining or assessing expression of the polypeptides and/or polynucleotides, whereby differences in expression from an index sample, or a sample taken from a subject prior to the initiation of the therapy, or during the course of therapy, is indicative of the efficacy, or optimal activity of the therapy.

In another embodiment, this invention provides an isolated nucleic acid molecule encoding for a splice variant according to the present invention, having a nucleotide sequence as set forth in any one of the sequences listed herein, or a sequence complementary thereto. In another embodiment, this invention provides an isolated nucleic acid molecule, having a nucleotide sequence as set forth in any one of the sequences listed herein, or a sequence complementary thereto. In another embodiment, this invention provides an oligonucleotide of at least about 12 nucleotides, specifically hybridizable with the nucleic acid molecules of this invention. In another embodiment, this invention provides vectors, cells, liposomes and compositions comprising the isolated nucleic acids of this invention.

In another embodiment, this invention provides a method for detecting a splice variant nucleic acid sequence in a biological sample, comprising: hybridizing the isolated nucleic acid molecules or oligonucleotide fragments of at least about 12 nucleotides thereof to a nucleic acid material of a biological sample and detecting a hybridization complex; wherein the presence of a hybridization complex correlates with the presence of a splice variant nucleic acid sequence in the biological sample.

According to optional but preferred embodiments of the present invention, any marker according to the present invention may optionally be used alone or combination. Such a combination may optionally comprise a plurality of markers described herein, optionally including any subcombination of markers, and/or a combination featuring at least one other marker, for example a known marker. Furthermore, such a combination may optionally and preferably be used as described above with regard to determining a ratio between a quantitative or semi-quantitative measurement of any marker described herein to any other marker described herein, and/or any other known marker, and/or any other marker. With regard to such a ratio between any marker described herein (or a combination thereof) and a known marker, more preferably the known marker comprises the “known protein” as described in greater detail below with regard to each cluster or gene.

According to preferred embodiments of the present invention, there is provided a method for detecting a Marker-detectable disease, comprising detecting specific expression of a splice variant as described herein.

According to preferred embodiments of the present invention, there is provided a method for screening for variant-detectable disease, comprising detecting cells affected by a Marker-detectable disease with a biomarker or a method or assay as described herein.

According to preferred embodiments of the present invention, there is provided a method for diagnosing a marker-detectable disease, comprising detecting cells affected by Marker-detectable disease with a biomarker or a method or assay according to any of the above claims.

According to preferred embodiments of the present invention, there is provided a method for monitoring disease progression and/or treatment efficacy and/or relapse of Marker-detectable disease, comprising detecting cells affected by Marker-detectable disease with a biomarker or a method or assay according to any of the above claims.

According to preferred embodiments of the present invention, there is provided a method of selecting a therapy for a marker-detectable disease, comprising detecting cells affected by a marker-detectable disease with a biomarker or a method or assay according to any of the above claims and selecting a therapy according to said detection.

There is also optionally provided a method for screening for variant-detectable disease, comprising detecting cells affected by a Marker-detectable disease with a biomarker or a method or assay according to any of the above embodiments.

There is also optionally provided a method for diagnosing a marker-detectable disease, comprising detecting cells affected by Marker-detectable disease with a biomarker or a method or assay according to any of the above embodiments.

There is also optionally provided a method for monitoring disease progression and/or treatment efficacy and/or relapse of Marker-detectable disease, comprising detecting cells affected by Marker-detectable disease with a biomarker or a method or assay according to any of the above embodiments.

There is also optionally provided a method of selecting a therapy for a marker-detectable disease, comprising detecting cells affected by a marker-detectable disease with a biomarker or a method or assay according to any of the above embodiments and selecting a therapy according to said detection.

According to some embodiments of the present invention, any of the above nucleic acid and/or amino acid sequences further comprises any sequence having at least about 70%, at least about 80%, at least about 90%, least about 95% homology to the polynucleotides herein described.

The nucleic acid sequences and/or amino acid sequences shown herein as embodiments of the present invention relate, in some embodiments, to their isolated form, as isolated polynucleotides (including for all transcripts), oligonucleotides (including for all segments, amplicons and primers), peptides (including for all tails, bridges, insertions or heads, optionally including other antibody epitopes as described herein) and/or polypeptides (including for all proteins). It should be noted that the terms “oligonucleotide” and “polynucleotide”, or “peptide” and “polypeptide”, may optionally be used interchangeably.

All technical and scientific terms used herein should be understood to have the meaning commonly understood by a person skilled in the art to which this invention belongs, as well as any other specified description. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). All of these are hereby incorporated by reference as if fully set forth herein.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows a schematic description of the cancer biomarker selection engine.

FIG. 2 shows a schematic illustration, depicting grouping of transcripts of a given cluster based on presence or absence of unique sequence regions.

FIG. 3 shows a schematic summary of quantitative real-time PCR analysis.

FIG. 4 is schematic presentation of the oligonucleotide based microarray fabrication.

FIG. 5 is schematic summary of the oligonucleotide based microarray experimental flow.

FIG. 6 shows cancer and cell-line vs. normal tissue expression for cluster AA383349, demonstrating overexpression in epithelial malignant tumors.

FIG. 7 is a histogram showing over expression of the Homo sapiens protein phosphatase 2a, catalytic subunit, zeta isoform (PPP2CZ) transcripts for transcripts detectable by or according to seg34WT-AA383349 (SEQ ID NO: 358) in cancerous Breast samples relative to the normal samples. FIG. 7A shows the results on a testing panel as described in Table 1_{—}3, while FIG. 7B shows the results on an enlarged panel presented in Table 1_{—}7.

FIG. 8 shows the results of real time PCR measurement of expression of transcripts detectable by or according to AA383349_seg34WT (SEQ ID NO: 358). FIG. 8A shows the results on normal panel as described in Table 1_{—}9, while FIG. 8B shows the results on an enlarged panel presented in Table 1_{—}10.

FIG. 9 is a histogram showing over expression of the protein phosphatase 2a AA383349 transcripts which are detectable by amplicon as depicted in sequence name AA383349_seg32 (SEQ ID NO: 361) in normal and cancerous Breast tissues. FIG. 9A shows the results on a testing panel as described in Table 1_{—}3, while FIG. 9B shows the results on an enlarged panel presented in Table 1_{—}7.

FIG. 10 is a histogram showing Expression of protein phosphatase 2a AA383349 transcripts which are detectable by amplicon as depicted in sequence name AA383349_seg32 (SEQ ID NO: 361) in different normal tissues. FIG. 10A shows the results on normal panel as described in Table 1_{—}9, while FIG. 10B shows the results on an enlarged panel presented in Table 1_{—}10.

FIG. 11 shows cancer and cell-line vs. normal tissue expression of cluster AA583399, demonstrating overexpression in brain malignant tumors, epithelial malignant tumors, a mixture of malignant tumors from different tissues and gastric carcinoma.

FIG. 12 is a histogram showing over expression of the Homo sapiens myeloma overexpressed gene (in a subset of t(11;14) positive multiple myelomas) (MYEOV) transcripts detectable by or according to AA583399_seg1 (SEQ ID NO: 364) in cancerous Lung samples relative to the normal samples. FIG. 12A shows the results on a testing panel as described in Table 1_{—}2, while FIG. 12B shows the results on an enlarged panel presented in Table 1_{—}6.

FIG. 13 is a histogram showing over expression of the Homo sapiens myeloma overexpressed gene (in a subset of t(11;14) positive multiple myelomas) (MYEOV) transcripts in cancerous Ovary samples relative to the normal samples for transcripts detectable by or according to AA583399_seg1 (SEQ ID NO: 364).

FIG. 14 shows the result of expression of Homo sapiens myeloma overexpressed gene (in a subset of t(11;14) positive multiple myelomas) (MYEOV) AA583399 transcripts which are detectable by amplicon as depicted in sequence name AA583399_seg1 (SEQ ID NO: 364) in different normal tissues. FIG. 14A shows the results on normal panel as described in Table 1_{—}9, while FIGS. 14B-C show the results on an enlarged panel presented in Table 1_{—}10. FIG. 14B presents the expression of each sample relative to the median of the colon samples; FIG. 14C presents the expression of each sample relative to the median of the lung samples.

FIG. 15 is a histogram showing over expression of the Homo sapiens myeloma overexpressed gene (in a subset of t(11;14) positive multiple myelomas) (MYEOV) transcripts detectable by or according to AA583399 seg17 (SEQ ID NO: 367) in cancerous Lung samples relative to the normal samples. FIG. 15A shows the results on a testing panel as described in Table 1_{—}2, while FIG. 15B shows the results on an enlarged panel presented in Table 1_{—}6.

FIG. 16 is a histogram showing over expression of the Homo sapiens myeloma overexpressed gene (in a subset of t(11;14) positive multiple myelomas) (MYEOV) transcripts detectable by or according to AA583399_seg17 (SEQ ID NO: 367) in cancerous Ovary samples relative to the normal samples. FIG. 16A shows the results on a testing panel as described in Table 1_{—}1, while FIG. 16B shows the results on an enlarged panel presented in Table 1_{—}5.

FIG. 17 shows expression of Homo sapiens myeloma overexpressed gene (in a subset of t(11;14) positive multiple myelomas) (MYEOV) AA583399 transcripts which are detectable by amplicon as depicted in sequence name AA583399 seg17 (SEQ ID NO: 367) in different normal tissues. FIG. 17A shows the results on normal panel as described in Table 1_{—}9, while FIGS. 17B-C show the results on an enlarged panel presented in Table 1_{—}10. FIG. 17B presents the expression of each sample relative to the median of the lung samples; FIG. 17C presents the expression of each sample relative to the median of the colon samples.

FIG. 18 is a histogram showing over expression of the Homo sapiens myeloma overexpressed gene (in a subset of t(11;14) positive multiple myelomas) (MYEOV) transcripts detectable by or according to AA583399_seg30-32 (SEQ ID NO: 370) in cancerous Lung samples relative to the normal samples.

FIG. 19 is a histogram showing over expression of the Homo sapiens myeloma overexpressed gene (in a subset of t(11;14) positive multiple myelomas) (MYEOV) transcripts detectable by or according to AA583399_seg30-32 (SEQ ID NO: 370) in cancerous Ovary samples relative to the normal samples.

FIG. 20 shows Expression of Homo sapiens myeloma overexpressed gene (in a subset of t(11;14) positive multiple myelomas) (MYEOV) AA583399 transcripts which are detectable by amplicon as depicted in sequence name AA583399 seg30-32 (SEQ ID NO: 370) in different normal tissues.

FIG. 21 is a histogram showing over expression of Homo sapiens myeloma overexpressed gene (in a subset of t(11;14) positive multiple myelomas) (MYEOV) AA583399 transcripts which are detectable by amplicon as depicted in sequence name AA583399_seg17 (SEQ ID NO: 367) in normal and cancerous Colon tissues.

FIG. 22 is a histogram showing down regulation of Homo sapiens myeloma overexpressed gene (in a subset of t(11;14) positive multiple myelomas) (MYEOV) AA583399 transcripts which are detectable by amplicon as depicted in sequence name AA583399 seg17 (SEQ ID NO: 367) in cancerous Breast samples relative to the normal samples.

FIG. 23 shows cancer and cell-line vs. normal tissue expression for cluster H13410.

FIG. 24 is a histogram showing over expression of Homo sapiens maba1 transcripts detectable by or according to H13410_seg10WTF2R2 (SEQ ID NO: 234) in cancerous lung samples relative to the normal samples.

FIG. 25 is a histogram showing over expression of the Homo sapiens maba1 transcripts detectable by or according to H13410_seg10WTF2R2 (SEQ ID NO: 234) in cancerous Ovary samples relative to the normal samples. FIG. 25A shows the results on a testing panel as described in Table 1_{—}1, while FIGS. 25B and 25C show the results on an enlarged panel presented in Table 1_{—}5. In FIG. 25B the samples are arranged according to the patient's age, and in FIG. 25C samples are arranged according to the stage of cancer of the patient.

FIG. 26 shows expression of Homo sapiens maba1 H13410 transcripts which are detectable by amplicon as depicted in sequence name H13410_seg10WTF2R2 (SEQ ID NO: 234) in different normal tissues. FIG. 26A shows the results on normal panel as described in Table 1_{—}9, while FIGS. 26B-D show the results on an enlarged panel presented in Table 1_{—}10. FIG. 26B presents the expression of each sample relative to the median of the breast samples; FIG. 26C presents the expression of each sample relative to the median of the ovary samples, FIG. 26D presents the expression of each sample relative to the median of the lung samples.

FIG. 27 is a histogram showing over expression of the Homo sapiens maba1 H13410 transcripts which are detectable by amplicon as depicted in sequence name H13410_seg12 (SEQ ID NO: 237) in normal and cancerous Breast tissues. FIG. 27A shows the results on a testing panel as described in Table 1_{—}3, while FIGS. 27B and 27C show the results on an enlarged panel presented in Table 1_{—}7. In FIG. 27B the cancer samples are arranged according to the stage of the cancer from the patient, and in FIG. 27C the samples are arranged according to patient's age.

FIG. 28 is a histogram showing over expression of the Homo sapiens maba1 (SEQ ID NO: 133) H13410 transcripts which are detectable by amplicon as depicted in sequence name H13410_seg12 (SEQ ID NO: 237) in cancerous Lung samples relative to the normal samples.

FIG. 29 is a histogram showing over expression of the Homo sapiens maba1 (SEQ ID NO: 133) H13410 transcripts which are detectable by amplicon as depicted in sequence name H13410_seg33 (SEQ ID NO: 240) in normal and cancerous Breast tissues. FIG. 29A shows the results on a testing panel as described in Table 1_{—}3, while FIG. 29B show the results on an enlarged panel presented in Table 1_{—}7.

FIG. 30 is a histogram showing over expression of the Homo sapiens maba1 (SEQ ID NO: 133) H13410 transcripts which are detectable by amplicon as depicted in sequence name H13410_seg33 (SEQ ID NO: 240) in cancerous Lung samples relative to the normal samples.

FIG. 31 is a histogram showing over expression of the Expression of Homo sapiens maba1 H13410 transcripts which are detectable by amplicon as depicted in sequence name H13410_seg58 (SEQ ID NO: 243) in normal and cancerous Breast tissues. FIG. 31A shows the results on a testing panel as described in Table 1_{—}3, while FIG. 31B show the results on an enlarged panel presented in Table 1_{—}7. In FIG. 31B—the cancer samples are arranged according to the stage of the cancer from the patient, and in FIG. 31C the samples are arranged according to the age of the patient.

FIG. 32 is a histogram showing over expression of the Homo sapiens maba1 (SEQ ID NO: 133) H13410 transcripts which are detectable by amplicon as depicted in sequence name H13410_seg58 (SEQ ID NO: 243) in cancerous Lung samples relative to the normal samples.

FIG. 33 is a histogram showing the Expression of Homo sapiens maba1 H13410 transcripts which are detectable by amplicon as depicted in sequence name H13410_seg58 (SEQ ID NO: 243) in different normal tissues. FIG. 33A shows the results on normal panel as described in Table 1_{—}9, while FIGS. 33B-D show the results on an enlarged panel presented in Table 1_{—}10. FIG. 33B presents the expression of each sample relative to the median of the breast samples; FIG. 33C presents the expression of each sample relative to the median of the ovary samples, FIG. 33D presents the expression of each sample relative to the median of the lung samples.

FIG. 34 is a histogram showing the expression of Homo sapiens maba1 H13410 transcripts which are detectable by amplicon as depicted in sequence name H13410_seg12 (SEQ ID NO: 237) in different normal tissues. FIG. 34A shows the results on normal panel as described in Table 1_{—}9, while FIGS. 34B-D show the results on an enlarged panel presented in Table 1_{—}10. FIG. 34B presents the expression of each sample relative to the median of the breast samples; FIG. 34C presents the expression of each sample relative to the median of the ovary samples, FIG. 34D presents the expression of each sample relative to the median of the lung samples.

FIG. 35 is a histogram showing the expression of Homo sapiens maba1 H13410 transcripts which are detectable by amplicon as depicted in sequence name H13410_seg33 (SEQ ID NO: 240) in different normal tissues. FIG. 35A shows the results on normal panel as described in Table 1_{—}9, while FIGS. 35B-D show the results on an enlarged panel presented in Table 1_{—}10. FIG. 355B presents the expression of each sample relative to the median of the breast samples; FIG. 35C presents the expression of each sample relative to the median of the ovary samples, FIG. 35D presents the expression of each sample relative to the median of the lung samples.

FIG. 36 is a histogram showing the expression of Homo sapiens maba1 (KIAA1324) H13410 transcripts which are detectable by amplicon as depicted in sequence name H13410_seg12 (SEQ ID NO: 237) in normal and cancerous Ovary tissues. FIG. 36A shows the results on a testing panel as described in Table 1_{—}1, while FIGS. 36B-C show the results on an enlarged panel presented in Table 1_{—}5. In FIG. 36B the samples are arranged according to the patient's age, and in FIG. 36C samples are arranged according to the stage of the cancer from the patient.

FIG. 37 is a histogram showing the expression of Homo sapiens maba1 (KIAA1324) H13410 transcripts which are detectable by amplicon as depicted in sequence name H13410_seg33 (SEQ ID NO: 240) in normal and cancerous Ovary tissues. FIG. 37A shows the results on a testing panel as described in Table 1_{—}1, while FIGS. 37B-C show the results on an enlarged panel presented in Table 1_{—}5. In FIG. 37B the samples are arranged according to the patient's age, and in FIG. 37C samples are arranged according to the patient's stage.

FIG. 38 is a histogram showing the expression of Homo sapiens maba1 (KIAA1324) H13410 transcripts which are detectable by amplicon as depicted in sequence name H13410_seg58 (SEQ ID NO: 243) in normal and cancerous Ovary tissues. FIG. 38A shows the results on a testing panel as described in Table 1_{—}1, while FIGS. 388B-C show the results on an enlarged panel presented in Table 1_{—}5. In FIG. 388B the samples are arranged according to the patient's age, and in FIG. 38C samples are arranged according to the patient's stage.

FIG. 39 is a histogram showing over expression of the above-indicated Homo sapiens maba1 (KIAA1324) H13410 transcripts which are detectable by amplicon as depicted in sequence name H13410_junc20-27F2R2 (SEQ ID NO: 385) in cancerous Breast samples relative to the normal samples.

FIG. 40 is a histogram showing over expression of the above-indicated Homo sapiens maba1 (KIAA1324) H13410 transcripts which are detectable by amplicon as depicted in sequence name H13410_junc20-27F2R2 (SEQ ID NO: 385) in different normal samples.

FIG. 41 is a histogram showing over expression of the above-indicated Homo sapiens maba1 (KIAA1324) H13410 transcripts which are detectable by amplicon as depicted in sequence name H13410_junc20-27F2R2 (SEQ ID NO: 385) in cancerous Ovary samples relative to the normal samples.

FIG. 42 shows cancer and cell-line vs. normal tissue expression in cluster H61775, showing overexpression in brain malignant tumors and a mixture of malignant tumors from different tissues.

FIG. 43 is a histogram showing over expression of the Homo sapiens immunoglobulin superfamily, member 9 (IGSF9) transcripts detectable by or according to H61775 seg8 (SEQ ID NO: 267) in cancerous Breast samples relative to the normal samples. FIG. 43A shows the results on a testing panel as described in Table 1_{—}3, while FIG. 43B shows the results on an enlarged panel presented in Table 1_{—}7.

FIG. 44 is a histogram showing over expression of the Homo sapiens immunoglobulin superfamily, member 9 (IGSF9) transcripts detectable by or according to H61775_seg12WT (SEQ ID NO: 270) in cancerous Ovary samples relative to the normal samples. FIG. 44A shows the results on a testing panel as described in Table 1_{—}1, while FIG. 44B shows the results on an enlarged panel presented in Table 1_{—}5.

FIG. 45 is a histogram showing over expression of the Homo sapiens immunoglobulin superfamily, member 9 (IGSF9) transcripts detectable by or according to H61775_seg12WT (SEQ ID NO: 270) in cancerous Lung samples relative to the normal samples. FIG. 45A shows the results on a testing panel as described in Table 1_{—}2, while FIG. 45B shows the results on an enlarged panel presented in Table 1_{—}6.

FIG. 46 is a histogram showing over expression of the Homo sapiens immunoglobulin superfamily, member 9 (IGSF9) transcripts detectable by or according to H61775_seg12WT (SEQ ID NO: 270) in cancerous Breast samples relative to the normal samples.

FIG. 47 shows expression of Homo sapiens immunoglobulin superfamily, member 9 (IGSF9) H61775 transcripts which are detectable by amplicon as depicted in sequence name H61775_seg12WT (SEQ ID NO: 270) in different normal tissues. FIG. 47A shows the results on normal panel as described in Table 1_{—}9, while FIGS. 47B-C show the results on an enlarged panel presented in Table 10. FIG. 47B presents the expression of each sample relative to the median of the ovary samples; FIG. 47C presents the expression of each sample relative to the median of the lung samples.

FIG. 48 is a histogram showing the expression of Homo sapiens immunoglobulin superfamily, member 9 (IGSF9) H61775 transcripts which are detectable by amplicon as depicted in sequence name H61775_seg8F2R2 (SEQ ID NO: 267) in normal and cancerous Lung tissues.

FIG. 49 is a histogram showing the Expression of Homo sapiens immunoglobulin superfamily, member 9 (IGSF9) H61775 transcripts which are detectable by amplicon as depicted in sequence name H61775_seg8F2R2 (SEQ ID NO: 267) in different normal tissues.

FIG. 50 is a histogram showing the Expression of Homo sapiens immunoglobulin superfamily, member 9 (IGSF9) H61775 transcripts which are detectable by amplicon as depicted in sequence name H61775_seg8F2R2 (SEQ ID NO: 267) in normal and cancerous Ovary tissues.

FIG. 51 is a histogram showing expression of Homo sapiens immunoglobulin superfamily, member 9 (IGSF9) H61775 transcripts which are detectable by amplicon as depicted in sequence name H61775_seg5-6F1R1 (SEQ ID NO: 394) in normal and cancerous Ovary tissues.

FIG. 52 is a histogram showing expression of Homo sapiens immunoglobulin superfamily, member 9 (IGSF9) H61775 transcripts which are detectable by amplicon as depicted in sequence name H61775_seg5-6F1R1 (SEQ ID NO: 394) in normal and cancerous lung tissues.

FIG. 53 is a histogram showing expression of Homo sapiens immunoglobulin superfamily, member 9 (IGSF9) H61775 transcripts which are detectable by amplicon as depicted in sequence name H61775_seg5-6F1R1 (SEQ ID NO: 394) in different normal tissues.

FIG. 54 is a histogram showing expression of Homo sapiens immunoglobulin superfamily, member 9 (IGSF9) H61775 transcripts which are detectable by amplicon as depicted in sequence name H61775_seg5-6F1R1 (SEQ ID NO: 394) in normal and cancerous breast tissues.

FIG. 55 shows cancer and cell-line vs. normal tissue expression for cluster HUMPAX8A, showing overexpression in ovarian carcinoma, a mixture of malignant tumors from different tissues, uterine malignancies and epithelial malignant tumors.

FIG. 56 is a histogram showing over-expression of Homo sapiens paired box gene 8 (PAX8)HUMPAX8A transcripts which are detectable by amplicon as depicted in sequence name HUMPAX8A seg23 (SEQ ID NO: 397) in normal and cancerous ovary tissues. FIG. 56A shows the results on a testing panel as described in Table 1_{—}1, while FIG. 56B shows the results on an enlarged panel presented in Table 1_{—}5.

FIG. 57 is a histogram showing over-expression of Homo sapiens paired box gene 8 (PAX8)HUMPAX8A transcripts which are detectable by amplicon as depicted in sequence name HUMPAX8A seg23 (SEQ ID NO: 397) in different normal tissues.

FIG. 58 shows cancer and cell-line vs. normal tissue expression for cluster N31842, showing overexpression in a mixture of malignant tumors from different tissues and epithelial malignant tumors.

FIG. 59 is a histogram showing over expression of the Homo sapiens homeo box C10 (HOXC10) transcripts detectable by or according to N31842_seg8WT (SEQ ID NO: 400) in cancerous lung samples relative to the normal samples. FIG. 59A shows the results on a testing panel as described in Table 1_{—}2, while FIG. 59B shows the results on an enlarged panel presented in Table 1_{—}6.

FIG. 60 shows expression of Homo sapiens homeo box C10 (HOXC10) N31842 transcripts which are detectable by amplicon as depicted in sequence name N31842_seg8 (SEQ ID NO: 400) in different normal tissues.

FIG. 61 is a histogram showing over expression of the Homo sapiens homeo box C10(HOXC10) transcripts detectable by or according to N31842_seg8 (SEQ ID NO: 400) in cancerous breast samples relative to the normal samples.

FIG. 62 is a histogram showing over expression of the Homo sapiens homeo box C10 (HOXC10) transcripts detectable by or according to N31842_seg6 (SEQ ID NO: 403) in cancerous Breast samples relative to the normal samples. FIG. 62A shows the results on a testing panel as described in Table 1_{—}3, while

FIG. 62B shows the results on an enlarged panel presented in Table 1_{—}7.

FIG. 63 shows expression of Homo sapiens homeo box C10 (HOXC10) N3 1842 transcripts which are detectable by amplicon as depicted in sequence name N31842_seg6 (SEQ ID NO: 403) in different normal tissues.

FIG. 63A shows the results on normal panel as described in Table 1_{—}9, while FIG. 63B shows the results on an enlarged panel presented in Table 1_{—}10.

FIG. 64 is a histogram showing overexpression of Homo sapiens homeo box C10 (HOXC10) N31842 transcripts which are detectable by amplicon as depicted in sequence name N31842_-seg6 (SEQ ID NO: 403) in normal and cancerous ovary tissues.

FIG. 65 is a histogram showing overexpression of Homo sapiens homeo box C10 (HOXC10) N31842 transcripts which are detectable by amplicon as depicted in sequence name N31842_seg6 (SEQ ID NO: 403) in normal and cancerous lung tissues.

FIG. 66 is a histogram showing over expression of the Homo sapiens LEM domain containing 1 (LEMD1) T58132 transcripts which are detectable by amplicon as depicted in sequence name T58132_seg6F2R2 (SEQ ID NO: 273) in normal and cancerous colon tissues. FIG. 66A shows the results on a testing panel as described in Table 1_{—}4, while FIG. 66B shows the results on an enlarged panel presented in Table 1_{—}8.

FIG. 67 is a histogram showing expression of Homo sapiens LEM domain containing 1 (LEMD1) T58132 transcripts which are detectable by amplicon as depicted in sequence name T58132_seg6F2R2 (SEQ ID NO: 273) in different normal tissues. FIG. 67A shows the results on normal panel as described in Table 1_{—}9, while FIGS. 67B-C show the results on an enlarged panel presented in Table 1_{—}10. FIG. 67B presents the expression of each sample relative to the median of the ovary samples; FIG. 67C presents the expression of each sample relative to the median of the colon samples.

FIG. 68 is a histogram showing over expression of the Homo sapiens LEM domain containing 1 (LEMD1) T58132 transcripts which are detectable by amplicon as depicted in sequence name T58132_seg8F2R2 (SEQ ID NO: 276) in normal and cancerous Colon tissues. FIG. 68A shows the results on a testing panel as described in Table 1_{—}4, while FIG. 689B shows the results on an enlarged panel presented in Table 1_{—}8.

FIG. 69 is a histogram showing over expression of the Homo sapiens LEM domain containing 1 (LEMD1) T58132 transcripts which are detectable by amplicon as depicted in sequence name T58132_seg21 (SEQ ID NO: 279) in normal and cancerous Colon tissues. FIG. 69A shows the results on a testing panel as described in Table 1_{—}4, while FIG. 69B shows the results on an enlarged panel presented in Table 1_{—}8.

FIG. 70 is a histogram showing the expression of Homo sapiens LEM domain containing 1 (LEMD1) T58132 transcripts which are detectable by amplicon as depicted in sequence name T58132_seg6F2R2 (SEQ ID NO: 273) in normal and cancerous Lung tissues. FIG. 70A shows the results on a testing panel as described in Table 1_{—}2, while FIG. 70B shows the results on an enlarged panel presented in Table 1_{—}6.

FIG. 71 is a histogram showing the expression of Homo sapiens LEM domain containing 1 (LEMD1) T58132 transcripts which are detectable by amplicon as depicted in sequence name T58132_seg6F2R2 (SEQ ID NO: 273) in normal and cancerous ovarian tissues. FIG. 71A shows the results on a testing panel as described in Table 1_{—}1, while FIG. 71B shows the results on an enlarged panel presented in Table 1_{—}5.

FIG. 72A is a histogram showing the expression of Homo sapiens LEM domain containing 1 (LEMD1) T58132 transcripts which are detectable by amplicon as depicted in sequence name T58132_seg8F2R2 (SEQ ID NO: 276) in normal and cancerous Lung tissues. FIG. 72A shows the results on a testing panel as described in Table 1_{—}2, while FIG. 72B shows the results on an enlarged panel presented in Table 1_{—}6.

FIG. 73 is a histogram showing the expression of Homo sapiens LEM domain containing 1 (LEMD1) T58132 transcripts which are detectable by amplicon as depicted in sequence name T58132_seg8F2R2 (SEQ ID NO: 276) in different normal tissues. FIG. 73A shows the results on normal panel as described in Table 1_{—}9, while FIGS. 73B-C show the results on an enlarged panel presented in Table 1_{—}10. FIG. 73B presents the expression of each sample relative to the median of the ovary samples; FIG. 73C presents the expression of each sample relative to the median of the colon samples.

FIG. 74 is a histogram showing the expression of Homo sapiens LEM domain containing 1 (LEMD1) T58132 transcripts which are detectable by amplicon as depicted in sequence name T58132_seg8F2R2 (SEQ ID NO: 276) in normal and cancerous ovary tissues. FIG. 74A shows the results on a testing panel as described in Table 1_{—}1, while FIG. 74B shows the results on an enlarged panel presented in Table 1_{—}5.

FIG. 75 is a histogram showing the expression of Homo sapiens LEM domain containing 1 (LEMD1) T58132 transcripts which are detectable by amplicon as depicted in sequence name T58132_seg21 (SEQ ID NO: 279) in normal and cancerous Lung tissues. FIG. 75A shows the results on a testing panel as described in Table 1_{—}2, while FIG. 75B shows the results on an enlarged panel presented in Table 1_{—}6.

FIG. 76 is a histogram showing the expression of Homo sapiens LEM domain containing 1 (LEMD1) T58132 transcripts which are detectable by amplicon as depicted in sequence name T58132_seg21 (SEQ ID NO: 279) in different normal tissues. FIG. 76A shows the results on normal panel as described in Table 1_{—}9, while FIGS. 76B-C show the results on an enlarged panel presented in Table 1_{—}10. FIG. 76B presents the expression of each sample relative to the median of the ovary samples; FIG. 76C presents the expression of each sample relative to the median of the colon samples.

FIG. 77 is a histogram showing the expression of Homo sapiens LEM domain containing 1 (LEMD1) T58132 transcripts which are detectable by amplicon as depicted in sequence name T58132_seg21 (SEQ ID NO: 279) in normal and cancerous ovary tissues. FIG. 77A shows the results on a testing panel as described in Table 1_{—}1, while FIG. 77B shows the results on an enlarged panel presented in Table 1_{—}5.

FIG. 78 shows cancer and cell-line vs. normal tissue expression for cluster T86235, showing overexpression in brain malignant tumors, a mixture of malignant tumors from different tissues, skin malignancies and epithelial malignant tumors.

FIG. 79 is a histogram showing over expression of the Homo sapiens trophinin associated protein (tastin) (TROAP) transcripts detectable by or according to T86235_seg44 (SEQ ID NO: 285) in cancerous Breast samples relative to the normal samples. FIG. 79A shows the results on a testing panel as described in Table 1_{—}3, while

FIG. 79B shows the results on an enlarged panel presented in Table 1_{—}7.

FIG. 80 is a histogram showing over expression of the Homo sapiens trophinin associated protein (tastin) (TROAP) transcripts detectable by or according to T86235_seg44 (SEQ ID NO: 285) in cancerous Ovary samples relative to the normal samples. FIG. 80A shows the results on a testing panel as described in Table 1_{—}1, while

FIG. 80B shows the results on an enlarged panel presented in Table 1_{—}5.

FIG. 81 shows expression of Homo sapiens trophinin associated protein (tastin) (TROAP) T86235 transcripts which are detectable by amplicon as depicted in sequence name T86235_seg44 (SEQ ID NO: 285) in different normal tissues. FIG. 81A shows the results on normal panel as described in Table 1_{—}9, while FIGS. 81B-D show the results on an enlarged panel presented in Table 1_{—}10. FIG. 81B presents the expression of each sample relative to the median of the lung samples; FIG. 81C presents the expression of each sample relative to the median of the ovary samples; FIG. 81D presents the expression of each sample relative to the median of the breast samples.

FIG. 82 is a histogram showing over expression of the Homo sapiens trophinin associated protein (tastin) (TROAP) transcripts detectable by or according to T86235_seg46-47WT (SEQ ID NO: 288) in cancerous Breast samples relative to the normal samples. FIG. 82 shows the results on a testing panel as described in Table 1_{—}3, while FIG. 82B shows the results on an enlarged panel presented in Table 1_{—}7.

FIG. 83 is a histogram showing over expression of the Homo sapiens trophinin associated protein (tastin) (TROAP) transcripts detectable by or according to T86235_seg46-47WT (SEQ ID NO: 288) in cancerous Lung samples relative to the normal samples. FIG. 83A shows the results on a testing panel as described in Table 1_{—}2, while FIG. 83B shows the results on an enlarged panel presented in Table 1_{—}6.

FIG. 84 is a histogram showing over expression of the Homo sapiens trophinin associated protein (tastin) (TROAP) transcripts detectable by or according to T86235_seg46-47WT (SEQ ID NO: 288) in cancerous Ovary samples relative to the normal samples. FIG. 84A shows the results on a testing panel as described in Table 1_{—}1, while FIG. 84B shows the results on an enlarged panel presented in Table 1_{—}5.

FIG. 85 shows expression of Homo sapiens trophinin associated protein (tastin) (TROAP) T86235 transcripts which are detectable by amplicon as depicted in sequence name T86235_seg46-47WT (SEQ ID NO: 288) in different normal tissues. FIG. 85A shows the results on normal panel as described in Table 1_{—}9, while FIGS. 85B-D show the results on an enlarged panel presented in Table 1_{—}10. FIG. 85B presents the expression of each sample relative to the median of the lung samples; FIG. 85C presents the expression of each sample relative to the median of the ovary samples; FIG. 85D presents the expression of each sample relative to the median of the breast samples.

FIG. 86 is a histogram showing over expression of the Homo sapiens trophinin associated protein (tastin) (TROAP) T86235 transcripts which are detectable by amplicon as depicted in sequence name T86235_seg44 (SEQ ID NO: 285) in normal and cancerous Lung tissues.

### DESCRIPTION OF EMBODIMENTS

The present invention provides, in some embodiments, polynucleotides and polypeptides and uses thereof, as further described herein. Polynucleotides and polypeptides described herein, in some embodiments, represent variants, which may optionally be used as diagnostic markers.

In some embodiments, these variants are useful as diagnostic markers for certain diseases, and as such the term “marker-detectable” or “variant-detectable” with regard to diseases is to be understood as encompassing use of the described polynucleotides and/or polypeptides.

In some embodiments, certain diseases are associated with differential expression, qualitatively or quantitatively, of the polynucleotides and polypeptides of this invention. Assessment of such expression, in turn, may in some embodiments, serve as a marker for a particular disease state, susceptibility, pathogenesis, etc., including any desired disease-specific event, whose analysis is useful, as will be appreciated by one skilled in the art. In one embodiment, such use as a marker is also referred to herein as the polynucleotides and polypeptides being “variant disease markers”.

The polynucleotides and polypeptides of the present invention, alone or in combination, in some embodiments, can be used for, and in some embodiments are a part of the methods of prognosis, prediction, screening, early diagnosis, staging, therapy selection and treatment monitoring of a marker-detectable disease. For example, in some embodiments, these markers may be used for the staging of disease in a patient (for example if the disease features cancer) and/or monitoring the progression of the disease. Furthermore, the markers of the present invention, alone or in combination, can be used for detection of the source of metastasis found in anatomical places other than the originating tissue, again in the example of cancer. Also, one or more of the markers may optionally be used in combination with one or more other disease markers (other than those described herein).

Biomolecular sequences (amino acid and/or nucleic acid sequences) uncovered using the methodology of the present invention and described herein can be efficiently utilized, in some embodiments, as tissue or pathological markers and/or as drugs or drug targets for treating or preventing a disease.

In some embodiments, these markers are released to the bloodstream under conditions of a particular disease, and/or are otherwise expressed at a much higher level and/or specifically expressed in tissue or cells afflicted with or demonstrating the disease. In some embodiments, the measurement of these markers, alone or in combination, in patient samples provides information that the diagnostician can correlate with a probable diagnosis of a particular disease and/or a condition that is indicative of a higher risk for a particular disease.

The present invention provides, in some embodiments, diagnostic assays for a marker-detectable disease and/or an indicative condition, and methods of use of such markers for detection of marker-detectable disease and/or an indicative condition, for example in a sample taken from a subject (patient), which in some embodiments, is a blood sample.

Some embodiments of this invention have been exemplified herein wherein cellular localization was determined via the use of four different software programs: (i) tmhmm (from Center for Biological Sequence Analysis, Technical University of Denmark DTU, www.cbs.dtu.dk/services/TMHMM/TMHMM2.0b.guide.php) or (ii) tmpred (from EMBnet, maintained by the ISREC Bionformatics group and the LICR Information Technology Office, Ludwig Institute for Cancer Research, Swiss Institute of Bioinformatics, www.ch.embnet.org/software/TMPRED_form.html) for transmembrane region prediction; (iii) signalp_hmm or (iv) signalp_nn (both from Center for Biological Sequence Analysis, Technical University of Denmark DTU, www.cbs.dtu.dk/services/SignalP/background/prediction.php) for signal peptide prediction. The terms “signalp_hmm” and “signalp_nn” refer to two modes of operation for the program SignalP: hmm refers to Hidden Markov Model, while nn refers to neural networks. Localization was also determined through manual inspection of known protein localization and/or gene structure, and the use of heuristics by the individual inventor. In some cases for the manual inspection of cellular localization prediction inventors used the ProLoc computational platform [Einat Hazkani-Covo, Erez Levanon, Galit Rotman, Dan Graur and Amit Novik; (2004) “Evolution of multicellularity in metazoa: comparative analysis of the subcellular localization of proteins in Saccharomyces, Drosophila and Caenorhabditis.” Cell Biology International 2004; 28(3):171-8.], which predicts protein localization based on various parameters including, protein domains (e.g., prediction of trans-membranous regions and localization thereof within the protein), pI, protein length, amino acid composition, homology to pre-annotated proteins, recognition of sequence patterns which direct the protein to a certain organelle (such as, nuclear localization signal, NLS, mitochondria localization signal), signal peptide and anchor modeling and using unique domains from Pfam that are specific to a single compartment.

Information is given in the text with regard to SNPs (single nucleotide polymorphisms). A description of the abbreviations is as follows. “T->C”, for example, means that the SNP results in a change at the position given in the table from T to C. Similarly, “M->Q”, for example, means that the SNP has caused a change in the corresponding amino acid sequence, from methionine (M) to glutamine (Q). If, in place of a letter at the right hand side for the nucleotide sequence SNP, there is a space, it indicates that a frameshift has occurred. A frameshift may also be indicated with a hyphen (-). A stop codon is indicated with an asterisk at the right hand side (*). As part of the description of an SNP, a comment may be found in parentheses after the above description of the SNP itself. This comment may include an FTId, which is an identifier to a SwissProt entry that was created with the indicated SNP. An FTId is a unique and stable feature identifier, which allows construction of links directly from position-specific annotation in the feature table to specialized protein-related databases. The FTId is always the last component of a feature in the description field, as follows: FTId=XXX_number, in which XXX is the 3-letter code for the specific feature key, separated by an underscore from a 6-digit number. In the table of the amino acid mutations of the wild type proteins of the selected splice variants of the invention, the header of the first column is “SNP position(s) on amino acid sequence”, representing a position of a known mutation on amino acid sequence. SNPs may optionally be used as diagnostic markers according to the present invention, alone or in combination with one or more other SNPs and/or any other diagnostic marker. Embodiments of the present invention comprise such SNPs, including but not limited to novel SNPs on the known (WT or wild type) protein sequences given below, as well as novel nucleic acid and/or amino acid sequences formed through such SNPs, and/or any SNP on a variant amino acid and/or nucleic acid sequence described herein.

Some embodiments of this invention have been exemplified herein wherein homology to known proteins was determined by Smith-Waterman version 5.1.2 using special (non default) parameters as follows:

model=sw.model

### GAPEXT=0

### GAPOP=100.0

MATRIX=blosum100

Some embodiments of this invention have been exemplified herein wherein overexpression of a cluster in cancer was a determination based on ESTs. A key to the p values with regard to the analysis of such overexpression is as follows:
library-based statistics: P-value without including the level of expression in cell-lines (P1)
library based statistics: P-value including the level of expression in cell-lines (P2)
EST clone statistics: P-value without including the level of expression in cell-lines (SP1)
EST clone statistics: predicted overexpression ratio without including the level of expression in cell-lines (R3)
EST clone statistics: P-value including the level of expression in cell-lines (SP2)
EST clone statistics: predicted overexpression ratio including the level of expression in cell-lines (R4)

Library-based statistics refer to statistics over an entire library, while EST clone statistics refer to expression only for ESTs from a particular tissue or cancer.

Some embodiments of this invention have been exemplified herein wherein overexpression of a cluster in cancer was a determination based on microarray use. As a microarray reference, in the specific segment paragraphs, the unabbreviated tissue name was used as the reference to the type of chip for which expression was measured. There are two types of microarray results: those from microarrays prepared according to a design by the present inventors, for which the microarray fabrication procedure is described in detail in Materials and Experimental Procedures section herein; and those results from microarrays using Affymetrix technology. As a microarray reference, in the specific segment paragraphs, the unabbreviated tissue name was used as the reference to the type of chip for which expression was measured. For microarrays prepared according to a design by the present inventors, the probe name begins with the name of the cluster (gene), followed by an identifying number. These probes are listed below with their respective sequences.

>HUMPAX8A_0_0_18307

(SEQ ID NO: 524)

TTTCCAGATGGATGGGTTCCAAAAGGGATGACAGAGGGACCTTCTGAGCT
>T86235_0_0_57380

(SEQ ID NO: 525)

AAGGCTTGGCTACAGTGCAAGGTTGGCTGAGCTGTGACAAGGTCTTCTCT
>T86235_0_0_57365

(SEQ ID NO: 526)

CATGGTAACACGGCCTCCATGGCTGAGTAGGGGACTAGGAAGGGTAAAAG
>HUMPAX8A_0_0_18296

(SEQ ID NO: 527)

AAAGACTCTGCAGCTCTGGCAGAACCACTAAGCTCTCAGCTCCTAGTCAT
>AA383349_0_8_0

(SEQ ID NO: 528)

TCCCTGAGGTACGAGTGTATGACCTGACACAATATGAGCACTGGCCAGAT
>T86235_0_7463

(SEQ ID NO: 492)

AGAAAGTAGCTCCAGCAGCACCCGAGAGGGTCAGGAGAAAAGCGGAGGAA
>N31842_0_5_42

(SEQ ID NO: 493)

CCTCCAGATGCCTGGCTTGGATGGCGTTGGAACAGGGCATTTGGAATGTT

Oligonucleotide microarray results taken from Affymetrix data were from chips available from Affymetrix Inc, Santa Clara, Calif., USA (see for example data regarding the Human Genome U133 (HG-U133) Set at www.affymetrix.com/products/arrays/specific/hgu133.affx; GeneChip Human Genome U133A 2.0 Array at www.affymetrix.com/products/arrays/specific/hgu133av2.affx; and Human Genome U133 Plus 2.0 Array at www.affymetrix.com/products/arrays/specific/hgu133plus.affx). The probe names follow the Affymetrix naming convention. The data is available from NCBI Gene Expression Omnibus (see www.ncbi.nlm.nih.gov/projects/geo/ and Edgar et al, Nucleic Acids Research, 2002, Vol. 30, No. 1 207-210). The dataset (including results) is available from www.ncbi.nlm.nih.gov/geo/query/acc.cgi?acc=GSE1133 for the Series GSE1133 database (published on March 2004); a reference to these results is as follows: Su et al (Proc Natl Acad Sci USA. 2004 Apr. 20; 101(16):6062-7. Epub 2004 Apr. 9).

Oligonucleotide microarray results taken from Affymetrix data were from chips available from Affymetrix Inc, Santa Clara, Calif., USA (see for example data regarding the Human Genome U133 (HG-U133) Set at www.affymetrix.com/products/arrays/specific/hgu133.affx; GeneChip Human Genome U133A 2.0 Array at www.affymetrix.com/products/arrays/specific/hgu133av2.affx; and Human Genome U133 Plus 2.0 Array at www.affymetrix.com/products/arrays/specific/hgu133plus.affx). The probe names follow the Affymetrix naming convention. The data is available from NCBI Gene Expression Omnibus (see www.ncbi.nlm.nih.gov/projects/geo/ and Edgar et al, Nucleic Acids Research, 2002, Vol. 30, No. 1 207-210). The dataset (including results) is available from www.ncbi.nlm.nih.gov/geo/query/acc.cgi?acc=GSE1133 for the Series GSE1133 database (published on March 2004); a reference to these results is as follows: Su et al (Proc Natl Acad Sci USA. 2004 Apr. 20; 101(16):6062-7. Epub 2004 Apr. 9).

The following list of abbreviations for tissues was used in the TAA histograms. The term “TAA” stands for “Tumor Associated Antigen”, and the TAA histograms, given in the text, represent the cancerous tissue expression pattern as predicted by the biomarkers selection engine, as described in detail in examples 1-5 below (the first word is the abbreviation while the second word is the full name):

(“BONE”, “bone”);
(“COL”, “colon”);
(“EPI”, “epithelial”);
(“GEN”, “general”);
(“LIVER”, “liver”);
(“LUN”, “lung”);
(“LYMPH”, “lymph nodes”);
(“MARROW”, “bone marrow”);
(“OVA”, “ovary”);
(“PANCREAS”, “pancreas”);
(“PRO”, “prostate”);
(“STOMACH”, “stomach”);
(“TCELL”, “T cells”);

### (“THYROID”, “Thyroid”);

(“MAM”, “breast”);
(“BRAIN”, “brain”);
(“UTERUS”, “uterus”);
(“SKIN”, “skin”);
(“KIDNEY”, “kidney”);
(“MUSCLE”, “muscle”);
(“ADREN”, “adrenal”);
(“HEAD”, “head and neck”);
(“BLADDER”, “bladder”);

It should be noted that the terms “segment”, “seg” and “node” (abbreviated as “N” in the names of nodes) are used interchangeably in reference to nucleic acid sequences of the present invention, they refer to portions of nucleic acid sequences that were shown to have one or more properties as described herein. They are also the building blocks that were used to construct complete nucleic acid sequences as described in greater detail elsewhere herein. Optionally and preferably, they are examples of oligonucleotides which are embodiments of the present invention, for example as amplicons, hybridization units and/or from which primers and/or complementary oligonucleotides may optionally be derived, and/or for any other use.

In some embodiments, the phrase “disease” refers to its commonly understood meaning, and includes, inter alia, any type of pathology and/or damage, including both chronic and acute damage, as well as a progress from acute to chronic damage.

In some embodiments, the phrase “marker” in the context of the present invention refers to a nucleic acid fragment, a peptide, or a polypeptide, which is differentially present in a sample taken from patients (subjects) having one of the herein-described diseases or conditions, as compared to a comparable sample taken from subjects who do not have one the above-described diseases or conditions.

In some embodiments, the phrase “differentially present” refers to differences in the quantity or quality of a marker present in a sample taken from patients having one of the herein-described diseases or conditions as compared to a comparable sample taken from patients who do not have one of the herein-described diseases or conditions. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the other sample. It should be noted that if the marker is detectable in one sample and not detectable in the other, then such a marker can be considered to be differentially present. Optionally, a relatively low amount of up-regulation may serve as the marker, as described herein. One of ordinary skill in the art could easily determine such relative levels of the markers; further guidance is provided in the description of each individual marker below.

In some embodiments, the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

In some embodiments, the phrase “qualitative” when in reference to differences in expression levels of a polynucleotide, polypeptide or cluster as described herein, refers to the presence versus absence of expression, or in some embodiments, the temporal regulation of expression, or in some embodiments, the timing of expression, or in some embodiments, the variant expressed, or in some embodiments, any post-translational modifications to the expressed molecule, and others, as will be appreciated by one skilled in the art. In some embodiments, the phrase “quantitative” when in reference to differences in expression levels of a polynucleotide, polypeptide or cluster as described herein, refers to absolute differences in quantity of expression, as determined by any means, known in the art, or in other embodiments, relative differences, which may be statistically significant, or in some embodiments, when viewed as a whole or over a prolonged period of time, etc., indicate a trend in terms of differences in expression.

In some embodiments, the term “diagnosing” refers to classifying a disease or a symptom, determining a severity of the disease, monitoring disease progression, forecasting an outcome of a disease and/or prospects of recovery. The term “detecting” may also optionally encompass any of the above.

Diagnosis of a disease according to the present invention can, in some embodiments, be effected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject, as described in greater detail below.

In some embodiments, the term “level” refers to expression levels of RNA and/or protein or to DNA copy number of a marker of the present invention.

Typically the level of the marker in a biological sample obtained from the subject is different (i.e., increased or decreased) from the level of the same variant in a similar sample obtained from a healthy individual (examples of biological samples are described herein).

Numerous well known tissue or fluid collection methods can be utilized to collect the biological sample from the subject in order to determine the level of DNA, RNA and/or polypeptide of the variant of interest in the subject.

Examples include, but are not limited to, fine needle biopsy, needle biopsy, core needle biopsy and surgical biopsy (e.g., brain biopsy), and lavage. Regardless of the procedure employed, once a biopsy/sample is obtained the level of the variant can be determined and a diagnosis can thus be made.

Determining the level of the same variant in normal tissues of the same origin is preferably effected along-side to detect an elevated expression and/or amplification and/or a decreased expression, of the variant as opposed to the normal tissues.

In some embodiments, the term “test amount” of a marker refers to an amount of a marker in a subject's sample that is consistent with a diagnosis of a particular disease or condition. A test amount can be either in absolute amount (e.g., microgram/ml) or a relative amount (e.g., relative intensity of signals).

In some embodiments, the term “control amount” of a marker can be any amount or a range of amounts to be compared against a test amount of a marker. For example, a control amount of a marker can be the amount of a marker in a patient with a particular disease or condition or a person without such a disease or condition. A control amount can be either in absolute amount (e.g., microgram/ml) or a relative amount (e.g., relative intensity of signals).

In some embodiments, the term “detect” refers to identifying the presence, absence or amount of the object to be detected.

In some embodiments, the term “label” includes any moiety or item detectable by spectroscopic, photo chemical, biochemical, immunochemical, or chemical means. For example, useful labels include 32P, 35S, fluorescent dyes, electron-dense reagents, enzymes (e.g., as commonly used in an ELISA), biotin-streptavadin, dioxigenin, haptens and proteins for which antisera or monoclonal antibodies are available, or nucleic acid molecules with a sequence complementary to a target. The label often generates a measurable signal, such as a radioactive, chromogenic, or fluorescent signal, that can be used to quantify the amount of bound label in a sample. The label can be incorporated in or attached to a primer or probe either covalently, or through ionic, van der Waals or hydrogen bonds, e.g., incorporation of radioactive nucleotides, or biotinylated nucleotides that are recognized by streptavadin. The label may be directly or indirectly detectable. Indirect detection can involve the binding of a second label to the first label, directly or indirectly. For example, the label can be the ligand of a binding partner, such as biotin, which is a binding partner for streptavadin, or a nucleotide sequence, which is the binding partner for a complementary sequence, to which it can specifically hybridize. The binding partner may itself be directly detectable, for example, an antibody may be itself labeled with a fluorescent molecule. The binding partner also may be indirectly detectable, for example, a nucleic acid having a complementary nucleotide sequence can be a part of a branched DNA molecule that is in turn detectable through hybridization with other labeled nucleic acid molecules (see, e.g., P. D. Fahrlander and A. Klausner, Bio/Technology 6:1165 (1988)). Quantitation of the signal is achieved by, e.g., scintillation counting, densitometry, or flow cytometry.

Exemplary detectable labels, optionally and preferably for use with immunoassays, include but are not limited to magnetic beads, fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

“Immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” or “specifically interacts or binds” when referring to a protein or peptide (or other epitope), refers, in some embodiments, to a binding reaction that is determinative of the presence of the protein in a heterogeneous population of proteins and other biologics. Thus, under designated immunoassay conditions, the specified antibodies bind to a particular protein at least two times greater than the background (non-specific signal) and do not substantially bind in a significant amount to other proteins present in the sample. Specific binding to an antibody under such conditions may require an antibody that is selected for its specificity for a particular protein. For example, polyclonal antibodies raised to seminal basic protein from specific species such as rat, mouse, or human can be selected to obtain only those polyclonal antibodies that are specifically immunoreactive with seminal basic protein and not with other proteins, except for polymorphic variants and alleles of seminal basic protein. This selection may be achieved by subtracting out antibodies that cross-react with seminal basic protein molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

In another embodiment, the present invention relates to bridges, tails, heads and/or insertions, and/or analogs, homologs and derivatives of such peptides. Such bridges, tails, heads and/or insertions are described in greater detail below with regard to the Examples.

In some embodiments, the term “tail” refers to a peptide sequence at the end of an amino acid sequence that is unique to a splice variant according to the present invention. Therefore, a splice variant having such a tail may optionally be considered as a chimera, in that at least a first portion of the splice variant is typically highly homologous (often 100% identical) to a portion of the corresponding known protein, while at least a second portion of the variant comprises the tail.

In some embodiments, the term “bead” refers to a peptide sequence at the beginning of an amino acid sequence that is unique to a splice variant according to the present invention. Therefore, a splice variant having such a head may optionally be considered as a chimera, in that at least a first portion of the splice variant comprises the head, while at least a second portion is typically highly homologous (often 100% identical) to a portion of the corresponding known protein.

In some embodiments, the term “an edge portion” refers to a connection between two portions of a splice variant according to the present invention that were not joined in the wild type or known protein. An edge may optionally arise due to a join between the above “known protein” portion of a variant and the tail, for example, and/or may occur if an internal portion of the wild type sequence is no longer present, such that two portions of the sequence are now contiguous in the splice variant that were not contiguous in the known protein. A “bridge” may optionally be an edge portion as described above, but may also include a join between a head and a “known protein” portion of a variant, or a join between a tail and a “known protein” portion of a variant, or a join between an insertion and a “known protein” portion of a variant.

In some embodiments, a bridge between a tail or a head or a unique insertion, and a “known protein” portion of a variant, comprises at least about 10 amino acids, or in some embodiments at least about 20 amino acids, or in some embodiments at least about 30 amino acids, or in some embodiments at least about 40 amino acids, in which at least one amino acid is from the tail/head/insertion and at least one amino acid is from the “known protein” portion of a variant. In some embodiments, the bridge may comprise any number of amino acids from about 10 to about 40 amino acids (for example, 10, 11, 12, 13 . . . 37, 38, 39, 40 amino acids in length, or any number in between).

It should be noted that a bridge cannot be extended beyond the length of the sequence in either direction, and it should be assumed that every bridge description is to be read in such manner that the bridge length does not extend beyond the sequence itself.

Furthermore, bridges are described with regard to a sliding window in certain contexts below. For example, certain descriptions of the bridges feature the following format: a bridge between two edges (in which a portion of the known protein is not present in the variant) may optionally be described as follows: a bridge portion of CONTIG-NAME_P1 (representing the name of the protein), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise XX (2 amino acids in the center of the bridge, one from each end of the edge), having a structure as follows (numbering according to the sequence of CONTIG-NAME_P1): a sequence starting from any of amino acid numbers 49−x to 49 (for example); and ending at any of amino acid numbers 50+((n−2)−x) (for example), in which x varies from 0 to n−2. In this example, it should also be read as including bridges in which n is any number of amino acids between 10-50 amino acids in length. Furthermore, the bridge polypeptide cannot extend beyond the sequence, so it should be read such that 49−x (for example) is not less than 1, nor 50+((n−2)−x) (for example) greater than the total sequence length.

In another embodiment, this invention provides isolated nucleic acid molecules, which in some embodiments encode for splice variants, having a nucleotide sequence as set forth in any one of the sequences listed herein, being homologous to such sequences, at a percent as described herein, or a sequence complementary thereto. In another embodiment, this invention provides an oligonucleotide of at least about 12 nucleotides, which specifically hybridizes with the nucleic acid molecules of this invention. In another embodiment, this invention provides vectors, cells, liposomes and compositions comprising the isolated nucleic acids or polypeptides of this invention, as appropriate.

In another embodiment, this invention provides a method for detecting the polypeptides of this invention in a biological sample, comprising: contacting a biological sample with an antibody specifically recognizing a splice variant according to the present invention under conditions whereby the antibody specifically interacts with the splice variant in the biological sample but do not recognize known corresponding proteins (wherein the known protein is discussed with regard to its splice variant(s) in the Examples below), and detecting said interaction; wherein the presence of an interaction correlates with the presence of a splice variant in the biological sample.

In another embodiment, this invention provides a method for detecting a polynucleotide of this invention in a biological sample, comprising: hybridizing the isolated nucleic acid molecules or oligonucleotide fragments of at least about a minimum length to a nucleic acid material of a biological sample and detecting a hybridization complex; wherein the presence of a hybridization complex correlates with the presence of a the polynucleotide in the biological sample.

In some embodiments of the present invention, the polypeptides/polynucleotides described herein are non-limiting examples of markers for diagnosing marker-detectable disease and/or an indicative condition. Each polypeptide/polynucleotide marker of the present invention can be used alone or in combination, for various uses, including but not limited to, prognosis, prediction, screening, early diagnosis, determination of progression, therapy selection and treatment monitoring of marker-detectable disease and/or an indicative condition, including a transition from an indicative condition to marker-detectable disease.

According to some embodiments of the present invention, any marker according to the present invention may optionally be used alone or combination. Such a combination may optionally comprise a plurality of markers described herein, optionally including any subcombination of markers, and/or a combination featuring at least one other marker, for example a known marker. Furthermore, such a combination may optionally and preferably be used as described above with regard to determining a ratio between a quantitative or semi-quantitative measurement of any marker described herein to any other marker described herein, and/or any other known marker, and/or any other marker. With regard to such a ratio between any marker described herein (or a combination thereof) and a known marker, more preferably the known marker comprises the “known protein” as described in greater detail below with regard to each cluster or gene.

In some embodiments of the present invention, there are provided of methods, uses, devices and assays for the diagnosis of a disease or condition. Optionally a plurality of biomarkers (or markers) may be used with the present invention. The plurality of markers may optionally include a plurality of markers described herein, and/or one or more known markers. The plurality of markers is preferably then correlated with the disease or condition. For example, such correlating may optionally comprise determining the concentration of each of the plurality of markers, and individually comparing each marker concentration to a threshold level. Optionally, if the marker concentration is above or below the threshold level (depending upon the marker and/or the diagnostic test being performed), the marker concentration correlates with the disease or condition. Optionally and preferably, a plurality of marker concentrations correlate with the disease or condition.

Alternatively, such correlating may optionally comprise determining the concentration of each of the plurality of markers, calculating a single index value based on the concentration of each of the plurality of markers, and comparing the index value to a threshold level.

Also alternatively, such correlating may optionally comprise determining a temporal change in at least one of the markers, and wherein the temporal change is used in the correlating step.

Also alternatively, such correlating may optionally comprise determining whether at least “X” number of the plurality of markers has a concentration outside of a predetermined range and/or above or below a threshold (as described above). The value of “X” may optionally be one marker, a plurality of markers or all of the markers; alternatively or additionally, rather than including any marker in the count for “X”, one or more specific markers of the plurality of markers may optionally be required to correlate with the disease or condition (according to a range and/or threshold).

Also alternatively, such correlating may optionally comprise determining whether a ratio of marker concentrations for two markers is outside a range and/or above or below a threshold. Optionally, if the ratio is above or below the threshold level and/or outside a range, the ratio correlates with the disease or condition.

Optionally, a combination of two or more these correlations may be used with a single panel and/or for correlating between a plurality of panels.

Optionally, the method distinguishes a disease or condition with a sensitivity of at least 70% at a specificity of at least 85% when compared to normal subjects. As used herein, sensitivity relates to the number of positive (diseased) samples detected out of the total number of positive samples present; specificity relates to the number of true negative (non-diseased) samples detected out of the total number of negative samples present. Preferably, the method distinguishes a disease or condition with a sensitivity of at least 80% at a specificity of at least 90% when compared to normal subjects. More preferably, the method distinguishes a disease or condition with a sensitivity of at least 90% at a specificity of at least 90% when compared to normal subjects. Also more preferably, the method distinguishes a disease or condition with a sensitivity of at least 70% at a specificity of at least 85% when compared to subjects exhibiting symptoms that mimic disease or condition symptoms.

A marker panel may be analyzed in a number of fashions well known to those of skill in the art. For example, each member of a panel may be compared to a “normal” value, or a value indicating a particular outcome. A particular diagnosis/prognosis may depend upon the comparison of each marker to this value; alternatively, if only a subset of markers are outside of a normal range, this subset may be indicative of a particular diagnosis/prognosis. The skilled artisan will also understand that diagnostic markers, differential diagnostic markers, prognostic markers, time of onset markers, disease or condition differentiating markers, etc., may be combined in a single assay or device. Markers may also be commonly used for multiple purposes by, for example, applying a different threshold or a different weighting factor to the marker for the different purpose(s).

In one embodiment, the panels comprise markers for the following purposes: diagnosis of a disease; diagnosis of disease and indication if the disease is in an acute phase and/or if an acute attack of the disease has occurred; diagnosis of disease and indication if the disease is in a non-acute phase and/or if a non-acute attack of the disease has occurred; indication whether a combination of acute and non-acute phases or attacks has occurred; diagnosis of a disease and prognosis of a subsequent adverse outcome; diagnosis of a disease and prognosis of a subsequent acute or non-acute phase or attack; disease progression (for example for cancer, such progression may include for example occurrence or recurrence of metastasis).

The above diagnoses may also optionally include differential diagnosis of the disease to distinguish it from other diseases, including those diseases that may feature one or more similar or identical symptoms.

In certain embodiments, one or more diagnostic or prognostic indicators are correlated to a condition or disease by merely the presence or absence of the indicator(s). In other embodiments, threshold level(s) of a diagnostic or prognostic indicator(s) can be established, and the level of the indicator(s) in a patient sample can simply be compared to the threshold level(s). The sensitivity and specificity of a diagnostic and/or prognostic test depends on more than just the analytical “quality” of the test—they also depend on the definition of what constitutes an abnormal result. In practice, Receiver Operating Characteristic curves, or “ROC” curves, are typically calculated by plotting the value of a variable versus its relative frequency in “normal” and “disease” populations, and/or by comparison of results from a subject before, during and/or after treatment. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition.

The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

ROC curves can be used even when test results don't necessarily give an accurate number. As long as one can rank results, one can create an ROC curve. For example, results of a test on “disease” samples might be ranked according to degree (say 1=low, 2=normal, and 3=high). This ranking can be correlated to results in the “normal” population, and a ROC curve created. These methods are well known in the art (see for example Hanley et al., Radiology 143: 29-36 (1982), incorporated by reference as if fully set forth herein).

One or more markers may lack diagnostic or prognostic value when considered alone, but when used as part of a panel, such markers may be of great value in determining a particular diagnosis/prognosis. In some embodiments, particular thresholds for one or more markers in a panel are not relied upon to determine if a profile of marker levels obtained from a subject are indicative of a particular diagnosis/prognosis. Rather, the present invention may utilize an evaluation of the entire marker profile by plotting ROC curves for the sensitivity of a particular panel of markers versus 1-(specificity) for the panel at various cutoffs. In these methods, a profile of marker measurements from a subject is considered together to provide a global probability (expressed either as a numeric score or as a percentage risk) that an individual has had a disease, is at risk for developing such a disease, optionally the type of disease which the individual has had or is at risk for, and so forth etc. In such embodiments, an increase in a certain subset of markers may be sufficient to indicate a particular diagnosis/prognosis in one patient, while an increase in a different subset of markers may be sufficient to indicate the same or a different diagnosis/prognosis in another patient. Weighting factors may also be applied to one or more markers in a panel, for example, when a marker is of particularly high utility in identifying a particular diagnosis/prognosis, it may be weighted so that at a given level it alone is sufficient to signal a positive result. Likewise, a weighting factor may provide that no given level of a particular marker is sufficient to signal a positive result, but only signals a result when another marker also contributes to the analysis.

In some embodiments, markers and/or marker panels are selected to exhibit at least 70% sensitivity, more preferably at least 80% sensitivity, even more preferably at least 85% sensitivity, still more preferably at least 90% sensitivity, and most preferably at least 95% sensitivity, combined with at least 70% specificity, more preferably at least 80% specificity, even more preferably at least 85% specificity, still more preferably at least 90% specificity, and most preferably at least 95% specificity. In some embodiments, both the sensitivity and specificity are at least 75%, more preferably at least 80%, even more preferably at least 85%, still more preferably at least 90%, and most preferably at least 95%. Sensitivity and/or specificity may optionally be determined as described above, with regard to the construction of ROC graphs and so forth, for example.

According to some embodiments of the present invention, individual markers and/or combinations (panels) of markers may optionally be used for diagnosis of time of onset of a disease or condition. Such diagnosis may optionally be useful for a wide variety of conditions, preferably including those conditions with an abrupt onset.

The phrase “determining the prognosis” as used herein refers to methods by which the skilled artisan can predict the course or outcome of a condition in a patient. The term “prognosis” does not refer to the ability to predict the course or outcome of a condition with 100% accuracy, or even that a given course or outcome is more likely to occur than not. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. For example, in individuals not exhibiting the condition, the chance of a given outcome may be about 3%. In some embodiments, a prognosis is about a 5% chance of a given outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, and about a 95% chance. The term “about” in this context refers to +/−1%.

The skilled artisan will understand that associating a prognostic indicator with a predisposition to an adverse outcome is a statistical analysis. For example, a marker level of greater than 80 pg/mL may signal that a patient is more likely to suffer from an adverse outcome than patients with a level less than or equal to 80 pg/mL, as determined by a level of statistical significance. Additionally, a change in marker concentration from baseline levels may be reflective of patient prognosis, and the degree of change in marker level may be related to the severity of adverse events. Statistical significance is often determined by comparing two or more populations, and determining a confidence interval and/or a p value. See, e.g., Dowdy and Wearden, Statistics for Research, John Wiley & Sons, New York, 1983. In one embodiment the confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001. Exemplary statistical tests for associating a prognostic indicator with a predisposition to an adverse outcome are described hereinafter.

In other embodiments, a threshold degree of change in the level of a prognostic or diagnostic indicator can be established, and the degree of change in the level of the indicator in a patient sample can simply be compared to the threshold degree of change in the level. A preferred threshold change in the level for markers of the invention is about 5%, about 10%, about 15%, about 20%, about 25%, about 30%, about 50%, about 75%, about 100%, and about 150%. The term “about” in this context refers to +/−10%. In yet other embodiments, a “nomogram” can be established, by which a level of a prognostic or diagnostic indicator can be directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

Exemplary, non-limiting methods and systems for identification of suitable biomarkers for marker panels are now described. Methods and systems for the identification of one or more markers for the diagnosis, and in particular for the differential diagnosis, of disease have been described previously. Suitable methods for identifying markers useful for the diagnosis of disease states are described in detail in U.S. patent application no. 2004-0126767, entitled METHOD AND SYSTEM FOR DISEASE DETECTION USING MARKER COMBINATIONS, filed Dec. 27, 2002, hereby incorporated by reference in its entirety as if fully set forth herein. One skilled in the art will also recognize that univariate analysis of markers can be performed and the data from the univariate analyses of multiple markers can be combined to form panels of markers to differentiate different disease conditions.

In developing a panel of markers useful in diagnosis, data for a number of potential markers may be obtained from a group of subjects by testing for the presence or level of certain markers. The group of subjects is divided into two sets, and preferably the first set and the second set each have an approximately equal number of subjects. The first set includes subjects who have been confirmed as having a disease or, more generally, being in a first condition state. For example, this first set of patients may be those that have recently had a disease and/or a particular type of the disease. The confirmation of this condition state may be made through more rigorous and/or expensive testing, preferably according to a previously defined diagnostic standard. Hereinafter, subjects in this first set will be referred to as “diseased”.

The second set of subjects are simply those who do not fall within the first set. Subjects in this second set may be “non-diseased;” that is, normal subjects. Alternatively, subjects in this second set may be selected to exhibit one symptom or a constellation of symptoms that mimic those symptoms exhibited by the “diseased” subjects.

The data obtained from subjects in these sets includes levels of a plurality of markers. Preferably, data for the same set of markers is available for each patient. This set of markers may include all candidate markers which may be suspected as being relevant to the detection of a particular disease or condition. Actual known relevance is not required. Embodiments of the methods and systems described herein may be used to determine which of the candidate markers are most relevant to the diagnosis of the disease or condition. The levels of each marker in the two sets of subjects may be distributed across a broad range, e.g., as a Gaussian distribution. However, no distribution fit is required.

As noted above, a marker often is incapable of definitively identifying a patient as either diseased or non-diseased. For example, if a patient is measured as having a marker level that falls within the overlapping region, the results of the test will be useless in diagnosing the patient. An artificial cutoff may be used to distinguish between a positive and a negative test result for the detection of the disease or condition. Regardless of where the cutoff is selected, the effectiveness of the single marker as a diagnosis tool is unaffected. Changing the cutoff merely trades off between the number of false positives and the number of false negatives resulting from the use of the single marker. The effectiveness of a test having such an overlap is often expressed using a ROC (Receiver Operating Characteristic) curve as described above.

As discussed above, the measurement of the level of a single marker may have limited usefulness. The measurement of additional markers provides additional information, but the difficulty lies in properly combining the levels of two potentially unrelated measurements. In the methods and systems according to embodiments of the present invention, data relating to levels of various markers for the sets of diseased and non-diseased patients may be used to develop a panel of markers to provide a useful panel response. The data may be provided in a database such as Microsoft Access, Oracle, other SQL databases or simply in a data file. The database or data file may contain, for example, a patient identifier such as a name or number, the levels of the various markers present, and whether the patient is diseased or non-diseased.

Next, an artificial cutoff region may be initially selected for each marker. The location of the cutoff region may initially be selected at any point, but the selection may affect the optimization process described below. In this regard, selection near a suspected optimal location may facilitate faster convergence of the optimizer. In an embodiment method, the cutoff region is initially centered about the center of the overlap region of the two sets of patients. In one embodiment, the cutoff region may simply be a cutoff point. In other embodiments, the cutoff region may have a length of greater than zero. In this regard, the cutoff region may be defined by a center value and a magnitude of length. In practice, the initial selection of the limits of the cutoff region may be determined according to a pre-selected percentile of each set of subjects. For example, a point above which a pre-selected percentile of diseased patients are measured may be used as the right (upper) end of the cutoff range.

Each marker value for each patient may then be mapped to an indicator. The indicator is assigned one value below the cutoff region and another value above the cutoff region. For example, if a marker generally has a lower value for non-diseased patients and a higher value for diseased patients, a zero indicator will be assigned to a low value for a particular marker, indicating a potentially low likelihood of a positive diagnosis. In other embodiments, the indicator may be calculated based on a polynomial. The coefficients of the polynomial may be determined based on the distributions of the marker values among the diseased and non-diseased subjects.

The relative importance of the various markers may be indicated by a weighting factor. The weighting factor may initially be assigned as a coefficient for each marker. As with the cutoff region, the initial selection of the weighting factor may be selected at any acceptable value, but the selection may affect the optimization process. In this regard, selection near a suspected optimal location may facilitate faster convergence of the optimizer. In an embodiment method, acceptable weighting coefficients may range between zero and one, and an initial weighting coefficient for each marker may be assigned as 0.5. In one embodiment, the initial weighting coefficient for each marker may be associated with the effectiveness of that marker by itself. For example, a ROC curve may be generated for the single marker, and the area under the ROC curve may be used as the initial weighting coefficient for that marker.

Next, a panel response may be calculated for each subject in each of the two sets. The panel response is a function of the indicators to which each marker level is mapped and the weighting coefficients for each marker. One advantage of using an indicator value rather than the marker value is that an extraordinarily high or low marker levels do not change the probability of a diagnosis of diseased or non-diseased for that particular marker. Typically, a marker value above a certain level generally indicates a certain condition state. Marker values above that level indicate the condition state with the same certainty. Thus, an extraordinarily high marker value may not indicate an extraordinarily high probability of that condition state. The use of an indicator which is constant on one side of the cutoff region eliminates this concern.

The panel response may also be a general function of several parameters including the marker levels and other factors including, for example, race and gender of the patient. Other factors contributing to the panel response may include the slope of the value of a particular marker over time. For example, a patient may be measured when first arriving at the hospital for a particular marker. The same marker may be measured again an hour later, and the level of change may be reflected in the panel response. Further, additional markers may be derived from other markers and may contribute to the value of the panel response. For example, the ratio of values of two markers may be a factor in calculating the panel response.

Having obtained panel responses for each subject in each set of subjects, the distribution of the panel responses for each set may now be analyzed. An objective function may be defined to facilitate the selection of an effective panel. The objective function should generally be indicative of the effectiveness of the panel, as may be expressed by, for example, overlap of the panel responses of the diseased set of subjects and the panel responses of the non-diseased set of subjects. In this manner, the objective function may be optimized to maximize the effectiveness of the panel by, for example, minimizing the overlap.

In a some embodiment, the ROC curve representing the panel responses of the two sets of subjects may be used to define the objective function. For example, the objective function may reflect the area under the ROC curve. By maximizing the area under the curve, one may maximize the effectiveness of the panel of markers. In other embodiments, other features of the ROC curve may be used to define the objective function. For example, the point at which the slope of the ROC curve is equal to one may be a useful feature. In other embodiments, the point at which the product of sensitivity and specificity is a maximum, sometimes referred to as the “knee,” may be used. In an embodiment, the sensitivity at the knee may be maximized. In further embodiments, the sensitivity at a predetermined specificity level may be used to define the objective function. Other embodiments may use the specificity at a predetermined sensitivity level may be used. In still other embodiments, combinations of two or more of these ROC-curve features may be used.

It is possible that one of the markers in the panel is specific to the disease or condition being diagnosed. When such markers are present at above or below a certain threshold, the panel response may be set to return a “positive” test result. When the threshold is not satisfied, however, the levels of the marker may nevertheless be used as possible contributors to the objective function.

An optimization algorithm may be used to maximize or minimize the objective function. Optimization algorithms are well-known to those skilled in the art and include several commonly available minimizing or maximizing functions including the Simplex method and other constrained optimization techniques. It is understood by those skilled in the art that some minimization functions are better than others at searching for global minimums, rather than local minimums. In the optimization process, the location and size of the cutoff region for each marker may be allowed to vary to provide at least two degrees of freedom per marker. Such variable parameters are referred to herein as independent variables. In one embodiment, the weighting coefficient for each marker is also allowed to vary across iterations of the optimization algorithm. In various embodiments, any permutation of these parameters may be used as independent variables.

In addition to the above-described parameters, the sense of each marker may also be used as an independent variable. For example, in many cases, it may not be known whether a higher level for a certain marker is generally indicative of a diseased state or a non-diseased state. In such a case, it may be useful to allow the optimization process to search on both sides. In practice, this may be implemented in several ways. For example, in one embodiment, the sense may be a truly separate independent variable which may be flipped between positive and negative by the optimization process. Alternatively, the sense may be implemented by allowing the weighting coefficient to be negative.

The optimization algorithm may be provided with certain constraints as well. For example, the resulting ROC curve may be constrained to provide an area-under-curve of greater than a particular value. ROC curves having an area under the curve of 0.5 indicate complete randomness, while an area under the curve of 1.0 reflects perfect separation of the two sets. Thus, a minimum acceptable value, such as 0.75, may be used as a constraint, particularly if the objective function does not incorporate the area under the curve. Other constraints may include limitations on the weighting coefficients of particular markers. Additional constraints may limit the sum of all the weighting coefficients to a particular value, such as 1.0.

The iterations of the optimization algorithm generally vary the independent parameters to satisfy the constraints while minimizing or maximizing the objective function. The number of iterations may be limited in the optimization process. Further, the optimization process may be terminated when the difference in the objective function between two consecutive iterations is below a predetermined threshold, thereby indicating that the optimization algorithm has reached a region of a local minimum or a maximum.

Thus, the optimization process may provide a panel of markers including weighting coefficients for each marker and cutoff regions for the mapping of marker values to indicators. In order to develop lower-cost panels which require the measurement of fewer marker levels, certain markers may be eliminated from the panel. In this regard, the effective contribution of each marker in the panel may be determined to identify the relative importance of the markers. In one embodiment, the weighting coefficients resulting from the optimization process may be used to determine the relative importance of each marker. The markers with the lowest coefficients may be eliminated.

Individual panel response values may also be used as markers in the methods described herein. For example, a panel may be constructed from a plurality of markers, and each marker of the panel may be described by a function and a weighting factor to be applied to that marker (as determined by the methods described above). Each individual marker level is determined for a sample to be tested, and that level is applied to the predetermined function and weighting factor for that particular marker to arrive at a sample value for that marker. The sample values for each marker are added together to arrive at the panel response for that particular sample to be tested. For a “diseased” and “non-diseased” group of patients, the resulting panel responses may be treated as if they were just levels of another disease marker.

Measures of test accuracy may be obtained as described in Fischer et al., Intensive Care Med. 29: 1043-51, 2003 (hereby incorporated by reference as if fully set forth herein), and used to determine the effectiveness of a given marker or panel of markers. These measures include sensitivity and specificity, predictive values, likelihood ratios, diagnostic odds ratios, and ROC curve areas. As discussed above, suitable tests may exhibit one or more of the following results on these various measures: at least 75% sensitivity, combined with at least 75% specificity; ROC curve area of at least 0.7, more preferably at least 0.8, even more preferably at least 0.9, and most preferably at least 0.95; and/or a positive likelihood ratio (calculated as sensitivity/(1-specificity)) of at least 5, more preferably at least 10, and most preferably at least 20, and a negative likelihood ratio (calculated as (1-sensitivity)/specificity) of less than or equal to 0.3, more preferably less than or equal to 0.2, and most preferably less than or equal to 0.1.

According to embodiments of the present invention, a splice variant protein or a fragment thereof, or a splice variant nucleic acid sequence or a fragment thereof, may be featured as a biomarker for detecting marker-detectable disease and/or an indicative condition, such that a biomarker may optionally comprise any of the above.

According to still other embodiments, the present invention optionally and preferably encompasses any amino acid sequence or fragment thereof encoded by a nucleic acid sequence corresponding to a splice variant protein as described herein. Any oligopeptide or peptide relating to such an amino acid sequence or fragment thereof may optionally also (additionally or alternatively) be used as a biomarker, including but not limited to the unique amino acid sequences of these proteins that are depicted as tails, heads, insertions, edges or bridges. The present invention also optionally encompasses antibodies capable of recognizing, and/or being elicited by, such oligopeptides or peptides.

The present invention also optionally and preferably encompasses any nucleic acid sequence or fragment thereof, or amino acid sequence or fragment thereof, corresponding to a splice variant of the present invention as described above, optionally for any application.

Non-limiting examples of methods or assays are described below.

The present invention also relates to kits based upon such diagnostic methods or assays.

### Nucleic Acid Sequences and Oligonucleotides

Various embodiments of the present invention encompass nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or artificially induced, either randomly or in a targeted fashion.

The present invention encompasses nucleic acid sequences described herein; fragments thereof, sequences hybridizable therewith, sequences homologous thereto [e.g., at least 50%, at least 55%, at least 60%, at least 65%, at least 70%, at least 75%, at least 80%, at least 85%, at least 95% or more say 100% identical to the nucleic acid sequences set forth below], sequences encoding similar polypeptides with different codon usage, altered sequences characterized by mutations, such as deletion, insertion or substitution of one or more nucleotides, either naturally occurring or man induced, either randomly or in a targeted fashion. The present invention also encompasses homologous nucleic acid sequences (i.e., which form a part of a polynucleotide sequence of the present invention) which include sequence regions unique to the polynucleotides of the present invention.

In cases where the polynucleotide sequences of the present invention encode previously unidentified polypeptides, the present invention also encompasses novel polypeptides or portions thereof, which are encoded by the isolated polynucleotide and respective nucleic acid fragments thereof described hereinabove.

A “nucleic acid fragment” or an “oligonucleotide” or a “polynucleotide” are used herein interchangeably to refer to a polymer of nucleic acids. A polynucleotide sequence of the present invention refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is composed of genomic and cDNA sequences. A composite sequence can include some exonal sequences required to encode a polypeptide, as well as some intronic sequences interposing therebetween. The intronic sequences can be of any source, including of other genes, and typically will include conserved splicing signal sequences. Such intronic sequences may further include cis acting expression regulatory elements.

Preferred embodiments of the present invention encompass oligonucleotide probes.

An example of an oligonucleotide probe which can be utilized by the present invention is a single stranded polynucleotide which includes a sequence complementary to the unique sequence region of any variant according to the present invention, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Alternatively, an oligonucleotide probe of the present invention can be designed to hybridize with a nucleic acid sequence encompassed by any of the above nucleic acid sequences, particularly the portions specified above, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

Oligonucleotides used according to this aspect of the present invention are those having a length selected from a range of about 10 to about 200 bases preferably about 15 to about 150 bases, more preferably about 20 to about 100 bases, most preferably about 20 to about 50 bases. Preferably, the oligonucleotide of the present invention features at least 17, at least 18, at least 19, at least 20, at least 22, at least 25, at least 30 or at least 40, bases specifically hybridizable with the biomarkers of the present invention.

The oligonucleotides of the present invention may comprise heterocylic nucleosides consisting of purines and the pyrimidines bases, bonded in a 3′ to 5′ phosphodiester linkage.

Preferably used oligonucleotides are those modified at one or more of the backbone, internucleoside linkages or bases, as is broadly described hereinunder.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases particularly useful for increasing the binding affinity of the oligomeric compounds of the invention include 5-substituted pyrimidines, 6-azapyrimidines and N2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

It will be appreciated that oligonucleotides of the present invention may include further modifications for more efficient use as diagnostic agents and/or to increase bioavailability, therapeutic efficacy and reduce cytotoxicity.

To enable cellular expression of the polynucleotides of the present invention, a nucleic acid construct according to the present invention may be used, which includes at least a coding region of one of the above nucleic acid sequences, and further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Any suitable promoter sequence can be used by the nucleic acid construct of the present invention.

Preferably, the promoter utilized by the nucleic acid construct of the present invention is active in the specific cell population transformed. Examples of cell type-specific and/or tissue-specific promoters include promoters such as albumin that is liver specific, lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the transgene is transcribed from CMV promoter. Vectors derived from Mo-MuLV are also included such as pBabe, where the transgene will be transcribed from the 5′LTR promoter.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Variant Recombinant Expression Vectors and Host Cells

Another aspect of the invention pertains to vectors, preferably expression vectors, containing a nucleic acid encoding a variant protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, Gene Expression Technology Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., variant proteins, mutant forms of variant proteins, fusion proteins, etc.).

The recombinant expression vectors of the invention can be designed for production of variant proteins in prokaryotic or eukaryotic cells. For example, variant proteins can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, to the amino or carboxyl terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin, PreScission, TEV and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) and pTrcHis (Invitrogen Life Technologies) that fuse glutathione S-transferase (GST), maltose E binding protein, protein A or 6×His, respectively, to the target recombinant protein.

Examples of suitable inducible non-fusion E. coli expression vectors include pTrc (Amrann et al., (1988) Gene 69:301-315).

One strategy to maximize recombinant protein expression in E. coli is to express the protein in a host bacteria with an impaired capacity to proteolytically cleave the recombinant protein. See, e.g., Gottesman, Gene Expression Technology Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques. Another optional strategy to solve codon bias is by using BL21-codon plus bacterial strains (Invitrogen) or Rosetta bacterial strain (Novagen), as these strains contain extra copies of rare E. coli tRNA genes.

In another embodiment, the expression vector encoding for the variant protein is a yeast expression vector. Examples of vectors for expression in yeast Saccharomyces cerivisae include pYepSec1 (Baldari, et al., 1987. EMBO J. 6: 229-234), pMFa (Kujan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, variant protein can be produced in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195), pIRESpuro (Clontech), pUB6 (Invitrogen), pCEP4 (Invitrogen) pREP4 (Invitrogen), pcDNA3 (Invitrogen). When used in mammalian cells, the expression vector's control functions are often provided by viral regulatory elements. For example, commonly used promoters are derived from polyoma, adenovirus 2, cytomegalovirus, Rous Sarcoma Virus, and simian virus 40. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the □-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to mRNA encoding for variant protein. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, variant protein can be produced in bacterial cells such as E. coli, insect cells, yeast or mammalian cells (such as Chinese hamster ovary cells (CHO) or COS or 293 cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin, puromycin, blasticidin and methotrexate. Nucleic acids encoding a selectable marker can be introduced into a host cell on the same vector as that encoding variant protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) variant protein. Accordingly, the invention further provides methods for producing variant protein using the host cells of the invention. In one embodiment, the method comprises culturing the host cell of the present invention (into which a recombinant expression vector encoding variant protein has been introduced) in a suitable medium such that variant protein is produced. In another embodiment, the method further comprises isolating variant protein from the medium or the host cell.

For efficient production of the protein, it is preferable to place the nucleotide sequences encoding the variant protein under the control of expression control sequences optimized for expression in a desired host. For example, the sequences may include optimized transcriptional and/or translational regulatory sequences (such as altered Kozak sequences).

### Hybridization Assays

Detection of a nucleic acid of interest in a biological sample may optionally be effected by hybridization-based assays using an oligonucleotide probe (non-limiting examples of probes according to the present invention were previously described).

Traditional hybridization assays include PCR, RT-PCR, Real-time PCR, RNase protection, in-situ hybridization, primer extension, Southern blots (DNA detection), dot or slot blots (DNA, RNA), and Northern blots (RNA detection) (NAT type assays are described in greater detail below). More recently, PNAs have been described (Nielsen et al. 1999, Current Opin. Biotechnol. 10:71-75). Other detection methods include kits containing probes on a dipstick setup and the like.

Hybridization based assays which allow the detection of a variant of interest (i.e., DNA or RNA) in a biological sample rely on the use of oligonucleotides which can be 10, 15, 20, or 30 to 100 nucleotides long preferably from 10 to 50, more preferably from 40 to 50 nucleotides long.

Thus, the isolated polynucleotides (oligonucleotides) of the present invention are preferably hybridizable with any of the herein described nucleic acid sequences under moderate to stringent hybridization conditions.

Moderate to stringent hybridization conditions are characterized by a hybridization solution such as containing 10% dextrane sulfate, 1 M NaCl, 1% SDS and 5×10⁶ cpm ³²P labeled probe, at 65° C., with a final wash solution of 0.2×SSC and 0.1% SDS and final wash at 65° C. and whereas moderate hybridization is effected using a hybridization solution containing 10% dextrane sulfate, 1 M NaCl, 1% SDS and 5×10⁶ cpm ³²P labeled probe, at 65° C., with a final wash solution of 1×SSC and 0.1% SDS and final wash at 50° C.

More generally, hybridization of short nucleic acids (below 200 bp in length, e.g. 17-40 bp in length) can be effected using the following exemplary hybridization protocols which can be modified according to the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ; (ii) hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample.

Probes can be labeled according to numerous well known methods. Non-limiting examples of radioactive labels include 3H, 14C, 32P, and 35S, Non-limiting examples of detectable markers include ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe.

For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, Fluor X (Amersham) and others [e.g., Kricka et al. (1992), Academic Press San Diego, Calif] can be attached to the oligonucleotides.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods.

As commonly known, radioactive nucleotides can be incorporated into probes of the invention by several methods. Non-limiting examples of radioactive labels include ³H, ¹⁴C, ³²P, and ³⁵S.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays.

Probes of the invention can be utilized with naturally occurring sugar-phosphate backbones as well as modified backbones including phosphorothioates, dithionates, alkyl phosphonates and a-nucleotides and the like. Probes of the invention can be constructed of either ribonucleic acid (RNA) or deoxyribonucleic acid (DNA), and preferably of DNA.

### NAT Assays

Detection of a nucleic acid of interest in a biological sample may also optionally be effected by NAT-based assays, which involve nucleic acid amplification technology, such as PCR for example (or variations thereof such as real-time PCR for example).

As used herein, a “primer” defines an oligonucleotide which is capable of annealing to (hybridizing with) a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14 Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the q3 replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra).

The terminology “amplification pair” (or “primer pair”) refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

In one particular embodiment, amplification of a nucleic acid sample from a patient is amplified under conditions which favor the amplification of the most abundant differentially expressed nucleic acid. In one preferred embodiment, RT-PCR is carried out on an mRNA sample from a patient under conditions which favor the amplification of the most abundant mRNA. In another preferred embodiment, the amplification of the differentially expressed nucleic acids is carried out simultaneously. It will be realized by a person skilled in the art that such methods could be adapted for the detection of differentially expressed proteins instead of differentially expressed nucleic acid sequences.

The nucleic acid (i.e. DNA or RNA) for practicing the present invention may be obtained according to well known methods.

Oligonucleotide primers of the present invention may be of any suitable length, depending on the particular assay format and the particular needs and targeted genomes employed. Optionally, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

It will be appreciated that antisense oligonucleotides may be employed to quantify expression of a splice isoform of interest. Such detection is effected at the pre-mRNA level. Essentially the ability to quantitate transcription from a splice site of interest can be effected based on splice site accessibility. Oligonucleotides may compete with splicing factors for the splice site sequences. Thus, low activity of the antisense oligonucleotide is indicative of splicing activity.

The polymerase chain reaction and other nucleic acid amplification reactions are well known in the art (various non-limiting examples of these reactions are described in greater detail below). The pair of oligonucleotides according to this aspect of the present invention are preferably selected to have compatible melting temperatures (Tm), e.g., melting temperatures which differ by less than that 7° C., preferably less than 5° C., more preferably less than 4° C., most preferably less than 3° C., ideally between 3° C. and 0° C.

Polymerase Chain Reaction (PCR): The polymerase chain reaction (PCR), as described in U.S. Pat. Nos. 4,683,195 and 4,683,202 to Mullis and Mullis et al., is a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are said to be “PCR-amplified.”

Ligase Chain Reaction (LCR or LAR): The ligase chain reaction [LCR; sometimes referred to as “Ligase Amplification Reaction” (LAR)] has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation, and ligation amplify a short segment of DNA. LCR has also been used in combination with PCR to achieve enhanced detection of single-base changes: see for example Segev, PCT Publication No. WO9001069 A1 (1990). However, because the four oligonucleotides used in this assay can pair to form two short ligatable fragments, there is the potential for the generation of target-independent background signal. The use of LCR for mutant screening is limited to the examination of specific nucleic acid positions.

Self-Sustained Synthetic Reaction (3SR/NASBA): The self-sustained sequence replication reaction (3SR) is a transcription-based in vitro amplification system that can exponentially amplify RNA sequences at a uniform temperature. The amplified RNA can then be utilized for mutation detection. In this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ) Replicase: In this method, a probe which recognizes the sequence of interest is attached to the replicatable RNA template for Qβ replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37 degrees C.). This prevents the use of high temperature as a means of achieving specificity as in the LCR, the ligation event can be used to detect a mutation at the junction site, but not elsewhere.

A successful diagnostic method must be very specific. A straight-forward method of controlling the specificity of nucleic acid hybridization is by controlling the temperature of the reaction. While the 3SR/NASBA, and Qβ systems are all able to generate a large quantity of signal, one or more of the enzymes involved in each cannot be used at high temperature (i.e., >55 degrees C.). Therefore the reaction temperatures cannot be raised to prevent non-specific hybridization of the probes. If probes are shortened in order to make them melt more easily at low temperatures, the likelihood of having more than one perfect match in a complex genome increases. For these reasons, PCR and LCR currently dominate the research field in detection technologies.

The basis of the amplification procedure in the PCR and LCR is the fact that the products of one cycle become usable templates in all subsequent cycles, consequently doubling the population with each cycle. The final yield of any such doubling system can be expressed as: (1+X)ⁿ=y, where “X” is the mean efficiency (percent copied in each cycle), “n” is the number of cycles, and “y” is the overall efficiency, or yield of the reaction. If every copy of a target DNA is utilized as a template in every cycle of a polymerase chain reaction, then the mean efficiency is 100%. If 20 cycles of PCR are performed, then the yield will be 2²⁰, or 1,048,576 copies of the starting material. If the reaction conditions reduce the mean efficiency to 85%, then the yield in those 20 cycles will be only 1.85²⁰, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

Also, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Additional NAT tests are Fluorescense In Situ Hybridization (FISH) and Comparative Genomic Hybridization (CGH). Fluorescense In Situ Hybridization (FISH)—The test uses fluorescent single-stranded DNA probes which are complementary to the DNA sequences that are under examination (genes or chromosomes). These probes hybridize with the complementary DNA and allow the identification of the chromosomal location of genomic sequences of DNA.

Comparative Genomic Hybridization (CGH)—allows a comprehensive analysis of multiple DNA gains and losses in entire genomes. Genomic DNA from the tissue to be investigated and a reference DNA are differentially labeled and simultaneously hybridized in situ to normal metaphase chromosomes. Variations in signal intensities are indicative of differences in the genomic content of the tissue under investigation.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect.

A similar 3′-mismatch strategy is used with greater effect to prevent ligation in the LCR. Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

The direct detection method according to various preferred embodiments of the present invention may be, for example a cycling probe reaction (CPR) or a branched DNA analysis.

When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

Branched DNA: Branched DNA (bDNA), involves oligonucleotides with branched structures that allow each individual oligonucleotide to carry 35 to 40 labels (e.g., alkaline phosphatase enzymes). While this enhances the signal from a hybridization event, signal from non-specific binding is similarly increased.

The detection of at least one sequence change according to various preferred embodiments of the present invention may be accomplished by, for example restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis or Dideoxy fingerprinting (ddF).

The demand for tests which allow the detection of specific nucleic acid sequences and sequence changes is growing rapidly in clinical diagnostics. As nucleic acid sequence data for genes from humans and pathogenic organisms accumulates, the demand for fast, cost-effective, and easy-to-use tests for as yet mutations within specific sequences is rapidly increasing.

A handful of methods have been devised to scan nucleic acid segments for mutations. One option is to determine the entire gene sequence of each test sample (e.g., a bacterial isolate). For sequences under approximately 600 nucleotides, this may be accomplished using amplified material (e.g., PCR reaction products). This avoids the time and expense associated with cloning the segment of interest. However, specialized equipment and highly trained personnel are required, and the method is too labor-intense and expensive to be practical and effective in the clinical setting.

In view of the difficulties associated with sequencing, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (ICC). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes have 4 to 6 base-pair recognition sequences, and cleave too frequently for many large-scale DNA manipulations. Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered. Recently, endonucleases encoded by group I introns have been discovered that might have greater than 12 base-pair specificity, but again, these are few in number.

Allele specific oligonucleotide (ASO): If the change is not in a recognition sequence, then allele-specific oligonucleotides (ASOs), can be designed to hybridize in proximity to the mutated nucleotide, such that a primer extension or ligation event can bused as the indicator of a match or a mis-match. Hybridization with radioactively labeled allelic specific oligonucleotides (ASO) also has been applied to the detection of specific point mutations. The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes and gsp/gip oncogenes. Because of the presence of various nucleotide changes in multiple positions, the ASO method requires the use of many oligonucleotides to cover all possible oncogenic mutations.

With either of the techniques described above (i.e., RFLP and ASO), the precise location of the suspected mutation must be known in advance of the test. That is to say, they are inapplicable when one needs to detect the presence of a mutation within a gene or sequence of interest.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE. Attaching a GC clamp to one primer is critical to ensure that the amplified sequence has a low dissociation temperature. Modifications of the technique have been developed, using temperature gradients, and the method can be also applied to RNA:RNA duplexes.

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient. TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations.

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations. The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

According to a presently preferred embodiment of the present invention the step of searching for any of the nucleic acid sequences described here, in tumor cells or in cells derived from a cancer patient is effected by any suitable technique, including, but not limited to, nucleic acid sequencing, polymerase chain reaction, ligase chain reaction, self-sustained synthetic reaction, Qβ-Replicase, cycling probe reaction, branched DNA, restriction fragment length polymorphism analysis, mismatch chemical cleavage, heteroduplex analysis, allele-specific oligonucleotides, denaturing gradient gel electrophoresis, constant denaturant gel electrophoresis, temperature gradient gel electrophoresis and dideoxy fingerprinting.

Detection may also optionally be performed with a chip or other such device. The nucleic acid sample which includes the candidate region to be analyzed is preferably isolated, amplified and labeled with a reporter group. This reporter group can be a fluorescent group such as phycoerythrin. The labeled nucleic acid is then incubated with the probes immobilized on the chip using a fluidics station. describe the fabrication of fluidics devices and particularly microcapillary devices, in silicon and glass substrates.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Since the sequence and position of each probe immobilized on the chip is known, the identity of the nucleic acid hybridized to a given probe can be determined.

It will be appreciated that when utilized along with automated equipment, the above described detection methods can be used to screen multiple samples for a disease and/or pathological condition both rapidly and easily.

### Amino Acid Sequences and Peptides

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

Polypeptide products can be biochemically synthesized such as by employing standard solid phase techniques. Such methods include but are not limited to exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase polypeptide synthesis procedures are well known in the art and further described by John Morrow Stewart and Janis Dillaha Young, Solid Phase Peptide Syntheses (2nd Ed., Pierce Chemical Company, 1984).

Synthetic polypeptides can optionally be purified by preparative high performance liquid chromatography [Creighton T. (1983) Proteins, structures and molecular principles. WH Freeman and Co. N.Y.], after which their composition can be confirmed via amino acid sequencing.

In cases where large amounts of a polypeptide are desired, it can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

The present invention also encompasses polypeptides encoded by the polynucleotide sequences of the present invention, as well as polypeptides according to the amino acid sequences described herein. The present invention also encompasses homologues of these polypeptides, such homologues can be at least 50%, at least 55%, at least 60%, at least 65%, at least 70%, at least 75%, at least 80%, at least 85%, at least 95% or more say 100% homologous to the amino acid sequences set forth below, as can be determined using BlastP software of the National Center of Biotechnology Information (NCBI) using default parameters, optionally and preferably including the following: filtering on (this option filters repetitive or low-complexity sequences from the query using the Seg (protein) program), scoring matrix is BLOSUM62 for proteins, word size is 3, E value is 10, gap costs are 11, 1 (initialization and extension), and number of alignments shown is 50. Preferably, nucleic acid sequence homology/identity is determined by using BlastN software of the National Center of Biotechnology Information (NCBI) using default parameters, which preferably include using the DUST filter program, and also preferably include having an E value of 10, filtering low complexity sequences and a word size of 11. Finally, the present invention also encompasses fragments of the above described polypeptides and polypeptides having mutations, such as deletions, insertions or substitutions of one or more amino acids, either naturally occurring or artificially induced, either randomly or in a targeted fashion.

It will be appreciated that peptides identified according the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified. Further details in this respect are provided hereinunder.

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids. Non-conventional or modified amino acids can be incorporated in the polypeptides of this invention as well, as will be known to one skilled in the art.

Since the peptides of the present invention are preferably utilized in diagnostics which require the peptides to be in soluble form, the peptides of the present invention preferably include one or more non-natural or natural polar amino acids, including but not limited to serine and threonine which are capable of increasing peptide solubility due to their hydroxyl-containing side chain.

The peptides of the present invention are preferably utilized in a linear form, although it will be appreciated that in cases where cyclicization does not severely interfere with peptide characteristics, cyclic forms of the peptide can also be utilized.

The peptides of present invention can be biochemically synthesized such as by using standard solid phase techniques. These methods include exclusive solid phase synthesis well known in the art, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Synthetic peptides can be purified by preparative high performance liquid chromatography and the composition of which can be confirmed via amino acid sequencing.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463 and also as described above.

Antibodies:

“Antibody” refers to a polypeptide ligand that is preferably substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad-immunoglobulin variable region genes. Antibodies exist, e.g., as intact immunoglobulins or as a number of well characterized fragments produced by digestion with various peptidases. This includes, e.g., Fab′ and F(ab)′2 fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

The functional fragments of antibodies, such as Fab, F(ab′)2, and Fv that are capable of binding to macrophages, are described as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Monoclonal antibody development may optionally be performed according to any method that is known in the art. The method described below is provided for the purposes of description only and is not meant to be limiting in any way.

### Antibody Engineering in Phage Display Libraries:

Antibodies of this invention may be prepared through the use of phage display libraries, as is known in the art, for example, as described in PCT Application No. WO 94/18219, U.S. Pat. No. 6,096,551, both of which are hereby fully incorporated by reference. The method involves inducing mutagenesis in a complementarity determining region (CDR) of an immunoglobulin light chain gene for the purpose of producing light chain gene libraries for use in combination with heavy chain genes and gene libraries to produce antibody libraries of diverse and novel immuno-specificities. The method comprises amplifying a CDR portion of an immunoglobulin light chain gene by polymerase chain reaction (PCR) using a PCR primer oligonucleotide. The resultant gene portions are inserted into phagemids for production of a phage display library, wherein the engineered light chains are displayed by the phages, for example for testing their binding specificity.

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using Papain produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (1972)]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. A scFv antibody fragment is an engineered antibody derivative that includes heavy- and light chain variable regions joined by a peptide linker. The minimal size of antibody molecules are those that still comprise the complete antigen binding site. ScFv antibody fragments are potentially more effective than unmodified IgG antibodies. The reduced size of 27-30 kDa permits them to penetrate tissues and solid tumors more readily. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)]. Optionally, there may be 1, 2 or 3 CDRs of different chains, but preferably there are 3 CDRs of 1 chain. The chain could be the heavy or the light chain.

Humanized forms of non-human (e.g., murine) antibodies, are chimeric molecules of immunoglobulins, immunoglobulin chains or fragments thereof (such as Fv, Fab, Fab′, F(ab′) or other antigen-binding subsequences of antibodies) which contain minimal sequence derived from non-human immunoglobulin, or fragments thereof may comprise the antibodies of this invention. Humanized antibodies are well known in the art. Methods for humanizing non-human antibodies are well known in the art, for example, as described in Winter and co-workers [Jones et al., Nature, 321:522-525 (1986); Riechmann et al., Nature 332:323-327 (1988); Verhoeyen et al., Science, 239:1534-1536 (1988)], U.S. Pat. No. 4,816,567, Hoogenboom and Winter, J. Mol. Biol., 227:381 (1991); Marks et al., J. Mol. Biol., 222:581 (1991), Cole et al., Monoclonal Antibodies and Cancer Therapy, Alan R. Liss, p. 77 (1985), Boerner et al., J. Immunol., 147(1):86-95 (1991), U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995), all of which are incorporated herein by reference.

Preferably, the antibody of this aspect of the present invention specifically binds at least one epitope of the polypeptide variants of the present invention. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

Optionally, a unique epitope may be created in a variant due to a change in one or more post-translational modifications, including but not limited to glycosylation and/or phosphorylation, as described below. Such a change may also cause a new epitope to be created, for example through removal of glycosylation at a particular site.

An epitope according to the present invention may also optionally comprise part or all of a unique sequence portion of a variant according to the present invention in combination with at least one other portion of the variant which is not contiguous to the unique sequence portion in the linear polypeptide itself, yet which are able to form an epitope in combination. One or more unique sequence portions may optionally combine with one or more other non-contiguous portions of the variant (including a portion which may have high homology to a portion of the known protein) to form an epitope.

### Immunoassays

In another embodiment of the present invention, an immunoassay can be used to qualitatively or quantitatively detect and analyze markers in a sample. This method comprises: providing an antibody that specifically binds to a marker; contacting a sample with the antibody; and detecting the presence of a complex of the antibody bound to the marker in the sample.

To prepare an antibody that specifically binds to a marker, purified protein markers can be used. Antibodies that specifically bind to a protein marker can be prepared using any suitable methods known in the art.

After the antibody is provided, a marker can be detected and/or quantified using any of a number of well recognized immunological binding assays. Useful assays include, for example, an enzyme immune assay (EIA) such as enzyme-linked immunosorbent assay (ELISA), a radioimmune assay (RIA), a Western blot assay, or a slot blot assay see, e.g., U.S. Pat. Nos. 4,366,241; 4,376,110; 4,517,288; and 4,837,168). Generally, a sample obtained from a subject can be contacted with the antibody that specifically binds the marker.

Optionally, the antibody can be fixed to a solid support to facilitate washing and subsequent isolation of the complex, prior to contacting the antibody with a sample. Examples of solid supports include but are not limited to glass or plastic in the form of, e.g., a microtiter plate, a stick, a bead, or a microbead. Antibodies can also be attached to a solid support.

After incubating the sample with antibodies, the mixture is washed and the antibody-marker complex formed can be detected. This can be accomplished by incubating the washed mixture with a detection reagent. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

The immunoassay can be used to determine a test amount of a marker in a sample from a subject. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can optionally be determined by comparing to a standard. As noted above, the test amount of marker need not be measured in absolute units, as long as the unit of measurement can be compared to a control amount and/or signal.

Preferably used are antibodies which specifically interact with the polypeptides of the present invention and not with wild type proteins or other isoforms thereof, for example. Such antibodies are directed, for example, to the unique sequence portions of the polypeptide variants of the present invention, including but not limited to bridges, heads, tails and insertions described in greater detail below. Preferred embodiments of antibodies according to the present invention are described in greater detail with regard to the section entitled “Antibodies”.

Radio-immunoassay (RIA): In one version, this method involves precipitation of the desired substrate and in the methods detailed hereinbelow, with a specific antibody and radiolabelled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a calorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabelled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a calorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

### Radio-Imaging Methods

These methods include but are not limited to, positron emission tomography (PET) single photon emission computed tomography (SPECT). Both of these techniques are non-invasive, and can be used to detect and/or measure a wide variety of tissue events and/or functions, such as detecting cancerous cells for example. Unlike PET, SPECT can optionally be used with two labels simultaneously. SPECT has some other advantages as well, for example with regard to cost and the types of labels that can be used. For example, U.S. Pat. No. 6,696,686 describes the use of SPECT for detection of breast cancer, and is hereby incorporated by reference as if fully set forth herein.

### Display Libraries

According to still another aspect of the present invention there is provided a display library comprising a plurality of display vehicles (such as phages, viruses or bacteria) each displaying at least 6, at least 7, at least 8, at least 9, at least 10, 10-15, 12-17, 15-20, 15-30 or 20-50 consecutive amino acids derived from the polypeptide sequences of the present invention.

Methods of constructing such display libraries are well known in the art. Such methods are described in, for example, Young A C, et al., “The three-dimensional structures of a polysaccharide binding antibody to Cryptococcus neoformans and its complex with a peptide from a phage display library: implications for the identification of peptide mimotopes” J Mol Biol 1997 Dec. 12; 274(4):622-34; Giebel L B et al. “Screening of cyclic peptide phage libraries identifies ligands that bind streptavidin with high affinities” Biochemistry 1995 Nov. 28; 34(47):15430-5; Davies E L et al., “Selection of specific phage-display antibodies using libraries derived from chicken immunoglobulin genes” J Immunol Methods 1995 Oct. 12; 186(1):125-35; Jones C R T al. “Current trends in molecular recognition and bioseparation” J Chromatogr A 1995 Jul. 14; 707(1):3-22; Deng S J et al. “Basis for selection of improved carbohydrate-binding single-chain antibodies from synthetic gene libraries” Proc Natl Acad Sci USA 1995 May 23; 92(11):4992-6; and Deng S J et al. “Selection of antibody single-chain variable fragments with improved carbohydrate binding by phage display” J Biol Chem 1994 Apr. 1; 269(13):9533-8, which are incorporated herein by reference.

### Theranostics:

The term theranostics describes the use of diagnostic testing to diagnose the disease, choose the correct treatment regime according to the results of diagnostic testing and/or monitor the patient response to therapy according to the results of diagnostic testing. Theranostic tests can be used to select patients for treatments that are particularly likely to benefit them and unlikely to produce side-effects. They can also provide an early and objective indication of treatment efficacy in individual patients, so that (if necessary) the treatment can be altered with a minimum of delay. For example: DAKO and Genentech together created HercepTest and Herceptin (trastuzumab) for the treatment of breast cancer, the first theranostic test approved simultaneously with a new therapeutic drug. In addition to HercepTest (which is an immunohistochemical test), other theranostic tests are in development which use traditional clinical chemistry, immunoassay, cell-based technologies and nucleic acid tests. PPGx's recently launched TPMT (thiopurine S-methyltransferase) test, which is enabling doctors to identify patients at risk for potentially fatal adverse reactions to 6-mercaptopurine, an agent used in the treatment of leukemia. Also, Nova Molecular pioneered SNP genotyping of the apolipoprotein E gene to predict Alzheimer's disease patients' responses to cholinomimetic therapies and it is now widely used in clinical trials of new drugs for this indication. Thus, the field of theranostics represents the intersection of diagnostic testing information that predicts the response of a patient to a treatment with the selection of the appropriate treatment for that particular patient.

### Surrogate Markers:

A surrogate marker is a marker, that is detectable in a laboratory and/or according to a physical sign or symptom on the patient, and that is used in therapeutic trials as a substitute for a clinically meaningful endpoint. The surrogate marker is a direct measure of how a patient feels, functions, or survives which is expected to predict the effect of the therapy. The need for surrogate markers mainly arises when such markers can be measured earlier, more conveniently, or more frequently than the endpoints of interest in terms of the effect of a treatment on a patient, which are referred to as the clinical endpoints. Ideally, a surrogate marker should be biologically plausible, predictive of disease progression and measurable by standardized assays (including but not limited to traditional clinical chemistry, immunoassay, cell-based technologies, nucleic acid tests and imaging modalities).

Surrogate endpoints were used first mainly in the cardiovascular area. For example, antihypertensive drugs have been approved based on their effectiveness in lowering blood pressure. Similarly, in the past, cholesterol-lowering agents have been approved based on their ability to decrease serum cholesterol, not on the direct evidence that they decrease mortality from atherosclerotic heart disease. The measurement of cholesterol levels is now an accepted surrogate marker of atherosclerosis. In addition, currently two commonly used surrogate markers in HIV studies are CD4+ T cell counts and quantitative plasma HIV RNA (viral load). In some embodiments of this invention, the polypeptide/polynucleotide expression pattern may serve as a surrogate marker for a particular disease, as will be appreciated by one skilled in the art.

### Monoclonal Antibody Therapy:

In some embodiments, monoclonal antibodies are useful for the identification of cancer cells. In some embodiments, monoclonal antibody therapy is a form of passive immunotherapy useful in cancer treatment. Such antibodies may comprise naked monoclonal antibodies or conjugated monoclonal antibodies—joined to a chemotherapy drug, radioactive particle, or a toxin (a substance that poisons cells). In some embodiments, the former is directly cytotoxic to the target (cancer) cell, or in another embodiment, stimulates or otherwise participates in an immune response ultimately resulting in the lysis of the target cell.

In some embodiments, the conjugated monoclonal antibodies are joined to drugs, toxins, or radioactive atoms. They are used as delivery vehicles to take those substances directly to the cancer cells. The MAb acts as a homing device, circulating in the body until it finds a cancer cell with a matching antigen. It delivers the toxic substance to where it is needed most, minimizing damage to normal cells in other parts of the body. Conjugated MAbs are also sometimes referred to as “tagged,” “labeled,” or “loaded” antibodies. MAbs with chemotherapy drugs attached are generally referred to as chemolabeled. MAbs with radioactive particles attached are referred to as radiolabeled, and this type of therapy is known as radioimmunotherapy (RIT). MAbs attached to toxins are called immunotoxins.

An illustrative, non-limiting example is provided herein of a method of treatment of a patient with an antibody to a variant as described herein, such that the variant is a target of the antibody. A patient with breast cancer is treated with a radiolabeled humanized antibody against an appropriate breast cancer target as described herein. The patient is optionally treated with a dosage of labeled antibody ranging from 10 to 30 mCi. Of course any type of therapeutic label may optionally be used.

The following sections relate to Candidate Marker Examples. It should be noted that Table numbering is restarted within each Example, which starts with the words “Description for Cluster”.

The following sections relate to Candidate Marker Examples.

### CANDIDATE MARKER EXAMPLES SECTION

This Section relates to Examples of sequences according to the present invention, including illustrative methods of selection thereof with regard to cancer; other markers were selected as described below for the individual markers.

Description of the methodology undertaken to uncover the biomolecular sequences of the present invention

Human ESTs and cDNAs were obtained from GenBank versions 136 (Jun. 15, 2003 ftp.ncbi.nih.gov/genbank/release.notes/gb136.release.notes); NCBI genome assembly of April 2003; RefSeq sequences from June 2003; Genbank version 139 (December 2003); Human Genome from NCBI (Build 34) (from October 2003); and RefSeq sequences from December 2003. With regard to GenBank sequences, the human EST sequences from the EST (GBEST) section and the human mRNA sequences from the primate (GBPR1) section were used; also the human nucleotide RefSeq mRNA sequences were used (see for example www.ncbi.nlm.nih.gov/Genbank/GenbankOverview.html and for a reference to the EST section, see www.ncbi.nlm.nih.gov/dbEST/; a general reference to dbEST, the EST database in GenBank, may be found in Boguski et al, Nat. Genet. 1993 August; 4(4):332-3; all of which are hereby incorporated by reference as if fully set forth herein).

Novel splice variants were predicted using the LEADS clustering and assembly system as described in Sorek, R., Ast, G. & Graur, D. Alu-containing exons are alternatively spliced. Genome Res 12, 1060-7 (2002); U.S. Pat. No. 6,625,545; and U.S. patent application Ser. No. 10/426,002, published as US20040101876 on May 27, 2004; all of which are hereby incorporated by reference as if fully set forth herein. Briefly, the software cleans the expressed sequences from repeats, vectors and immunoglobulins. It then aligns the expressed sequences to the genome taking alternatively splicing into account and clusters overlapping expressed sequences into “clusters” that represent genes or partial genes.

These were annotated using the GeneCarta (Compugen, Tel-Aviv, Israel) platform. The GeneCarta platform includes a rich pool of annotations, sequence information (particularly of spliced sequences), chromosomal information, alignments, and additional information such as SNPs, gene ontology terms, expression profiles, functional analyses, detailed domain structures, known and predicted proteins and detailed homology reports.

A brief explanation is provided with regard to the method of selecting the candidates. However, it should be noted that this explanation is provided for descriptive purposes only, and is not intended to be limiting in any way. The potential markers were identified by a computational process that was designed to find genes and/or their splice variants that are specifically expressed in cardiac tissue, as opposed to other types of tissues and also particularly as opposed to muscle tissue, by using databases of expressed sequences. Various parameters related to the information in the EST libraries, determined according to classification by library annotation, were used to assist in locating genes and/or splice variants thereof that are specifically and/or differentially expressed in heart tissues. The detailed description of the selection method and of these parameters is presented in Example 1 below.

Selecting Candidates with Regard to Cancer

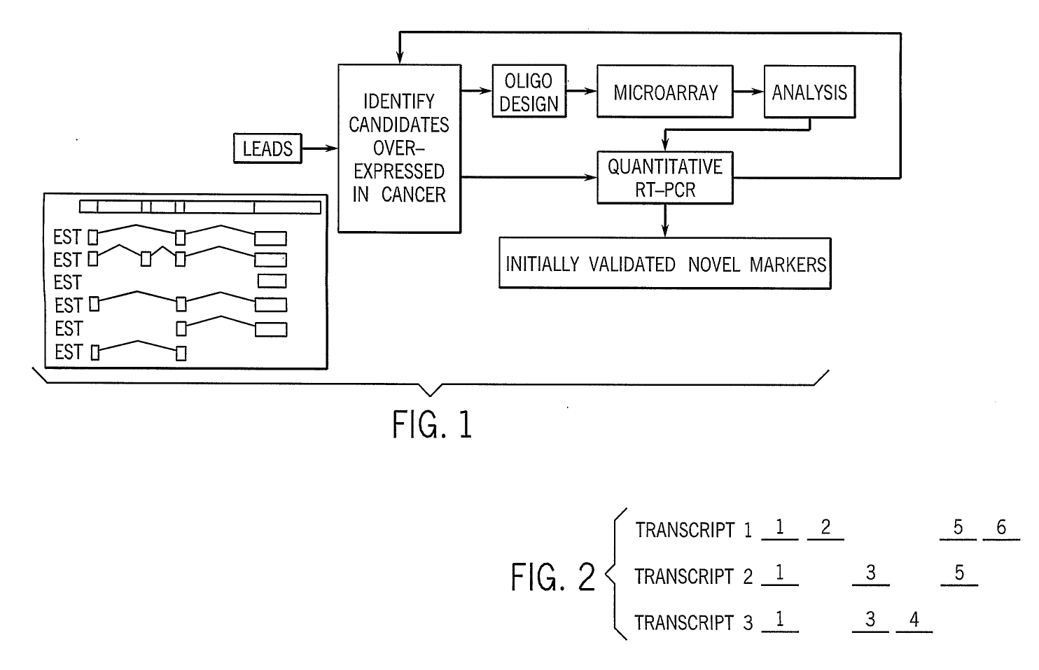

A brief explanation is provided with regard to a non-limiting method of selecting the candidates for cancer diagnostics. However, it should noted that this explanation is provided for descriptive purposes only, and is not intended to be limiting in any way. The potential markers were identified by a computational process that was designed to find genes and/or their splice variants that are over-expressed in tumor tissues, by using databases of expressed sequences. Various parameters related to the information in the EST libraries, determined according to a manual classification process, were used to assist in locating genes and/or splice variants thereof that are over-expressed in cancerous tissues. The detailed description of the selection method is presented in Example 1 below. The cancer biomarkers selection engine and the following wet validation stages are schematically summarized in FIG. 1.

### Example 1

### Identification of Differentially Expressed Gene Products

### Algorithm

In order to distinguish between differentially expressed gene products and constitutively expressed genes (i.e., house keeping genes) an algorithm based on an analysis of frequencies was configured. A specific algorithm for identification of transcripts over expressed in cancer is described hereinbelow.

Dry Analysis
Library annotation—EST libraries are manually classified according to:
(i) Tissue origin
(ii) Biological source—Examples of frequently used biological sources for construction of EST libraries include cancer cell-lines; normal tissues; cancer tissues; fetal tissues; and others such as normal cell lines and pools of normal cell-lines, cancer cell-lines and combinations thereof. A specific description of abbreviations used below with regard to these tissues/cell lines etc is given above.
(iii) Protocol of library construction—various methods are known in the art for library construction including normalized library construction; non-normalized library construction; subtracted libraries; ORESTES and others. It will be appreciated that at times the protocol of library construction is not indicated.

The following rules are followed:

EST libraries originating from identical biological samples are considered as a single library.

EST libraries which include above-average levels of DNA contamination are eliminated.

Dry computation—development of engines which are capable of identifying genes and splice variants that are temporally and spacially expressed.

Clusters (genes) having at least five sequences including at least two sequences from the tissue of interest are analyzed.

### Example 2

### Identification of Genes Over Expressed in Cancer

Two different scoring algorithms were developed.

Libraries score—candidate sequences which are supported by a number of cancer libraries, are more likely to serve as specific and effective diagnostic markers.

The basic algorithm—for each cluster the number of cancer and normal libraries contributing sequences to the cluster was counted. Fisher exact test was used to check if cancer libraries are significantly over-represented in the cluster as compared to the total number of cancer and normal libraries.

Library counting: Small libraries (e.g., less than 1000 sequences) were excluded from consideration unless they participate in the cluster. For this reason, the total number of libraries is actually adjusted for each cluster.

Clones no. score—Generally, when the number of ESTs is much higher in the cancer libraries relative to the normal libraries it might indicate actual over-expression.

The Algorithm—

Clone counting: For counting EST clones each library protocol class was given a weight based on an assessment of how much the protocol reflects actual expression levels:

(i) non-normalized: 1

(ii) normalized: 0.2

(iii) all other classes: 0.1

Clones number score—The total weighted number of EST clones from cancer libraries was compared to the EST clones from normal libraries. To avoid cases where one library contributes to the majority of the score, the contribution of the library that gives most clones for a given cluster was limited to 2 clones.

The score was computed as

$$\frac{c + 1}{C} / \frac{n + 1}{N}$$

where:

c—weighted number of “cancer” clones in the cluster.

C—weighted number of clones in all “cancer” libraries.

n—weighted number of “normal” clones in the cluster.

N—weighted number of clones in all “normal” libraries.

Clones number score significance—Fisher exact test was used to check if EST clones from cancer libraries are significantly over-represented in the cluster as compared to the total number of EST clones from cancer and normal libraries.

Two search approaches were used to find either general cancer-specific candidates or tumor specific candidates.
Libraries/sequences originating from tumor tissues are counted as well as libraries originating from cancer cell-lines (“normal” cell-lines were ignored).
Only libraries/sequences originating from tumor tissues are counted

### Example 3

### Identification of Tissue Specific Genes

For detection of tissue specific clusters, tissue libraries/sequences were compared to the total number of libraries/sequences in cluster. Similar statistical tools to those described in above were employed to identify tissue specific genes. Tissue abbreviations are the same as for cancerous tissues, but are indicated with the header “normal tissue”.

The algorithm—for each tested tissue T and for each tested cluster the following were examined:

1. Each cluster includes at least 2 libraries from the tissue T. At least 3 clones (weighed—as described above) from tissue T in the cluster; and

2. Clones from the tissue T are at least 40% from all the clones participating in the tested cluster

Fisher exact test P-values were computed both for library and weighted clone counts to check that the counts are statistically significant.

### Example 4

### Identification of Splice Variants Over Expressed in Cancer of Clusters which are not Over Expressed in Cancer

Cancer-specific splice variants containing a unique region were identified.

Identification of unique sequence regions in splice variants

A Region is defined as a group of adjacent exons that always appear or do not appear together in each splice variant.

A “segment” (sometimes referred also as “seg” or “node”) is defined as the shortest contiguous transcribed region without known splicing inside.

Only reliable ESTs were considered for region and segment analysis. An EST was defined as unreliable if:

(i) Unspliced;

(ii) Not covered by RNA;

(iii) Not covered by spliced ESTs; and

(iv) Alignment to the genome ends in proximity of long poly-A stretch or starts in proximity of long poly-T stretch.

Only reliable regions were selected for further scoring. Unique sequence regions were considered reliable if:

(i) Aligned to the genome; and

(ii) Regions supported by more than 2 ESTs.

The Algorithm

Each unique sequence region divides the set of transcripts into 2 groups:

(i) Transcripts containing this region (group TA).

(ii) Transcripts not containing this region (group TB).

The set of EST clones of every cluster is divided into 3 groups:

(i) Supporting (originating from) transcripts of group TA (S1).

(ii) Supporting transcripts of group TB (S2).

(iii) Supporting transcripts from both groups (S3).

Library and clones number scores described above were given to S1 group.

Fisher Exact Test P-values were used to check if:

S1 is significantly enriched by cancer EST clones compared to S2; and

S1 is significantly enriched by cancer EST clones compared to cluster background (S1+S2+S3).

Identification of unique sequence regions and division of the group of transcripts accordingly is illustrated in FIG. 2. Each of these unique sequence regions corresponds to a segment, also termed herein a “node”.

Region 1: common to all transcripts, thus it is not considered; Region 2: specific to Transcript 1: T_{—}1 unique regions (2+6) against T_{—}2+3 unique regions (3+4); Region 3: specific to Transcripts 2+3: T_{—}2+3 unique regions (3+4) against T1 unique regions (2+6); Region 4: specific to Transcript 3: T_{—}3 unique regions (4) against T1+2 unique regions (2+5+6); Region 5: specific to Transcript 1+2: T_{—}1+2 unique regions (2+5+6) against T3 unique regions (4); Region 6: specific to Transcript 1: same as region 2.

### Example 5

### Identification of Cancer Specific Splice Variants of Genes Over Expressed in Cancer

A search for EST supported (no mRNA) regions for genes of:

(i) known cancer markers

(ii) Genes shown to be over-expressed in cancer in published micro-array experiments.

Reliable EST supported-regions were defined as supported by minimum of one of the following:

(i) 3 spliced ESTs; or

(ii) 2 spliced ESTs from 2 libraries;

(iii) 10 unspliced ESTs from 2 libraries, or

(iv) 3 libraries.

### Example 6

Diseases and conditions that may be diagnosed with one or more variant(s) according to the present invention.

Various non-limiting examples are given below of cancerous conditions for which one or more variants according to the present invention may have a diagnostic, or therapeutic utility.

### Ovarian Cancer

Ovarian cancer causes more deaths than any other cancer of the female reproductive system, however, only 25% of ovarian cancers are detected in stage I. No single marker has been shown to be sufficiently sensitive or specific to contribute to the diagnosis of ovarian cancer.

In one embodiment, the markers of this invention are utilized alone, or in combination with other markers, for the diagnosis, treatment or assessment of prognosis of ovarian cancer. Such other markers may comprise CA-125 or mucin 16, CA-50, CA 54-61, CA-195 and CA 19-9, STN and TAG-72, kallikreins, cathepsin L, urine gonadotropin, inhibins, cytokeratins, such as TPA and TPS, members of the Transforming Growth Factors (TGF) beta superfamily, Epidermal Growth Factor, p53 and HER-2 or any combination thereof.

Immunohistochemistry may be used to assess the origin of the tumor and staging as part of the methods of this invention, and as protected uses for the polypeptides of this invention.

In some embodiments, this invention provides polypeptides/polynucleotides which serves as markers for ovarian cancer. In some embodiments, the marker is any polypeptide/polynucleotide as described herein. In some embodiments, the marker is an AA583399, H13410, H61775, T86235, HUMPAX8A, N31842, T58132, or variants as described herein or markers related thereto. Each variant marker of the present invention described herein may be used alone or in combination with one or more other variant ovarian cancer described herein, and/or in combination with known markers for ovarian cancer, as described herein. Diagnosis of ovarian cancer and or of other conditions that may be diagnosed by these markers or variants of them include but are not limited to the presence, risk and/or extent of the following:
1. The identification of a metastasis of unknown origin which originated from a primary ovarian cancer.
2. As a marker to distinguish between different types of ovarian cancer, therefore potentially affect treatment choice (e.g. discrimination between epithelial tumors and germ cell tumors).
3. As a tool in the assessment of abdominal mass and in particular in the differential diagnosis between a benign and malignant ovarian cysts.
4. As a tool for the assessment of infertility.
5. Other conditions that may elevate serum levels of ovary related markers. These include but are not limited to: cancers of the endometrium, cervix, fallopian tubes, pancreas, breast, lung and colon; nonmalignant conditions such as pregnancy, endometriosis, pelvic inflammatory disease and uterine fibroids.
6. Conditions which have similar symptoms, signs and complications as ovarian cancer and where the differential diagnosis between them and ovarian cancer is of clinical importance including but not limited to:
   a. Non-malignant causes of pelvic mass. Including, but not limited to: benign (functional) ovarian cyst, uterine fibroids, endometriosis, benign ovarian neoplasms and inflammatory bowel lesions
   b. Any condition suggestive of a malignant tumor including but not limited to anorexia, cachexia, weight loss, fever, hypercalcemia, skeletal or abdominal pain, paraneoplastic syndrome.
   c. Ascites.
7. Prediction of patient's drug response
8. As surrogate markers for clinical outcome of a treated cancer.

### Breast Cancer

Breast cancer is the most commonly occurring cancer in women, comprising almost a third of all malignancies in females. In one embodiment, the polypeptides and/or polynucleotides of this invention are utilized alone, or in combination with other markers, for the diagnosis, treatment or assessment of prognosis of breast cancer. In one embodiment, the polypeptides and/or polynucleotides serve as markers of disease.

Such markers may be used alone, or in combination with other known markers for breast cancer, including, inter alia, Mucin1 (measured as CA 15-3), CEA (CarcinoEmbryonic Antigen), HER-2, CA125, CA 19-9, PCNA, Ki-67, E-Cadherin, Cathepsin D, TFF1, epidermal growth factor receptor (EGFR), cyclin E, p53, bcl-2, vascular endothelial growth factor, urokinase-type plasminogen activator-1, survivin, or any combination thereof, and includes use of any compound which detects or quantifies the same. ESR (Erythrocyte Sedimentation Rate) values may be obtained, and comprise the marker panel for breast cancer.

In some embodiments, the polypeptides/polynucleotides of this invention serve as prognosticators, in identifying, inter alia, patients at minimal risk of relapse, patients with a worse prognosis, or patients likely to benefit from specific treatments.

There are some non-cancerous pathological conditions which represent an increased risk factor for development breast cancer, and as such, patients with these conditions may be evaluated using the polypeptides/polynucleotides and according to the methods of this invention, for example, as part of the screening methods of this invention, Some of these conditions include, but are not limited to ductal hyperplasia without atypia, atypical hyperplasia, and others.

In some embodiments, the polypeptides/polynucleotides of this invention serve as markers for breast cancer, including, but not limited to: AA383349, H13410, H61775, T86235, N31842 or homologues thereof. In some embodiments, the AA383349, H13410, H61775, T86235, N31842 or polynucleotides encoding the same, can be used alone or in combination with any other desired marker, including, inter alia, Calcitonin, CA15-3 (Mucin1), CA27-29, TPA, a combination of CA 15-3 and CEA, CA 27.29 (monoclonal antibody directed against MUC1), Estrogen 2 (beta), HER-2 (c-erbB2), or any combinations thereof.

In some embodiments, the polypeptides/polynucleotides of this invention may be useful in, inter alia, assessing the presence, risk and/or extent of the following:
1. The identification of a metastasis of unknown origin which originated from a primary breast cancer tumor.
2. In the assessment of lymphadenopathy, and in particular axillary lymphadenopathy.
3. As a marker to distinguish between different types of breast cancer, therefore potentially affect treatment choice (e.g. as HER-2)
4. As a tool in the assessment of palpable breast mass and in particular in the differential diagnosis between a benign and malignant breast mass.
5. As a tool in the assessment of conditions affecting breast skin (e.g. Paget's disease) and their differentiation from breast cancer.
6. As a tool in the assessment of breast pain or discomfort resulting from either breast cancer or other possible conditions (e.g. Mastitis, Mondors syndrome).
7. Other conditions not mentioned above which have similar symptoms, signs and complications as breast cancer and where the differential diagnosis between them and breast cancer is of clinical importance including but not limited to:
   a. Abnormal mammogram and/or nipple retraction and/or nipple discharge due to causes other than breast cancer. Such causes include but are not limited to benign breast masses, melanoma, trauma and technical and/or anatomical variations.
   b. Any condition suggestive of a malignant tumor including but not limited to anorexia, cachexia, weight loss, fever, hypercalcemia, paraneoplastic syndrome.

Lymphadenopathy, weight loss and other signs and symptoms associated with breast cancer but originate from diseases different from breast cancer including but not limited to other malignancies, infections and autoimmune diseases.
8. Prediction of patient's drug response
9. As surrogate markers for clinical outcome of a treated cancer.

### Lung Cancer

Lung cancer is the primary cause of cancer death among both men and women in the U.S. In one embodiment, the polypeptides and/or polynucleotides of this invention are utilized alone, or in combination with other markers, for the diagnosis, treatment or assessment of prognosis of lung cancer. In one embodiment, the term “lung cancer” is to be understood as encompassing small cell or non-small cell lung cancers, including adenocarcinomas, bronchoalveolar-alveolar, squamous cell and large cell carcinomas.

In some embodiments, the polypeptides/polynucleotides of this invention are utilized in conjunction with other screening procedures, as well as the use of other markers, for the diagnosis, or assessment of prognosis of lung cancer in a subject. In some embodiments, such screening procedures may comprise the use of chest x-rays, analysis of the type of cells contained in sputum, fiberoptic examination of the bronchial passages, or any combination thereof. Such evaluation in turn may impact the type of treatment regimen pursued, which in turn may reflect the type and stage of the cancer, and include surgery, radiation therapy and/or chemotherapy.

Current radiotherapeutic agents, chemotherapeutic agents and biological toxins are potent cytotoxins, yet do not discriminate between normal and malignant cells, producing adverse effects and dose-limiting toxicities. In some embodiments of this invention, the polypeptides/polynucleotides provide a means for more specific targeting to neoplastic versus normal cells.

In some embodiments, the polypeptides for use in the diagnosis, treatment and/or assessment of progression of lung cancer may comprise: AA583399, H13410, H61775, T86235, N31842, T58132 or homologoues thereof, or polynucleotides encoding the same. In some embodiments, these polypeptides/polynucleotides may be used alone or in combination with one or more other appropriate markers, including, inter alia, other polypeptides/polynucleotides of this invention. In some embodiments, such use may be in combination with other known markers for lung cancer, including but not limited to CEA, CA15-3, Beta-2-microglobulin, CA19-9, TPA, and/or in combination with native sequences associated with the polypeptides/polynucleotides of this invention, as herein described.

In some embodiments, the polypeptides/polynucleotides of this invention may be useful in, inter alia, assessing the presence, risk and/or extent of the following:
1. The identification of a metastasis of unknown origin which originated from a primary lung cancer.
2. The assessment of a malignant tissue residing in the lung that is from a non-lung origin, including but not limited to: osteogenic and soft tissue sarcomas; colorectal, uterine, cervix and corpus tumors; head and neck, breast, testis and salivary gland cancers; melanoma; and bladder and kidney tumors.
3. Distinguishing between different types of lung cancer, therefore potentially affect treatment choice (e.g. small cell vs. non small cell tumors).
4. Unexplained dyspnea and/or chronic cough and/or hemoptysis, and analysis thereof.
5. Differential diagnosis of the origin of a pleural effusion.
6. Conditions which have similar symptoms, signs and complications as lung cancer and where the differential diagnosis between them and lung cancer is of clinical importance including but not limited to:
   a. Non-malignant causes of lung symptoms and signs. Symptoms and signs include, but are not limited to: lung lesions and infiltrates, wheeze, stridor.
   b. Other symptoms, signs and complications suggestive of lung cancer, such as tracheal obstruction, esophageal compression, dysphagia, recurrent laryngeal nerve paralysis, hoarseness, phrenic nerve paralysis with elevation of the hemidiaphragm and Horner syndrome.
   c. Any condition suggestive of a malignant tumor including but not limited to anorexia, cachexia, weight loss, fever, hypercalcemia, hypophosphatemia, hyponatremia, syndrome of inappropriate secretion of antidiuretic hormone, elevated ANP, elevated ACTH, hypokalemia, clubbing, neurologic-myopathic syndromes and thrombophlebitis.
7. Prediction of patient's drug response
8. As surrogate markers for clinical outcome of a treated cancer.

### Colorectal Cancer:

Colon and rectal cancers are malignant conditions which occur in the corresponding segments of the large intestine. In one embodiment, the polypeptides and/or polynucleotides of this invention are utilized alone, or in combination with other markers, for the diagnosis, treatment or assessment of prognosis of colorectal cancer. In some embodiments, the term “colorectal cancers” is to be understood as encompassing adenocarcinomas, carcinoid tumors, for example, found in the appendix and rectum; gastrointestinal stromal tumors for example, found in connective tissue in the wall of the colon and rectum; and lymphomas, which are malignancies of immune cells in the colon, rectum and lymph nodes. In some embodiments, the polypeptides/polynucleotides are useful in diagnosing, treating and/or assessing progression of colorectal pathogenesis, including the maturation of adenomatous polyps, to larger polyps, and all relevant stages in the neoplastic transformation of the tissue.

In some embodiments, the polypeptides/polynucleotides of this invention are utilized in conjunction with other screening procedures, as well as the use of other markers, for the diagnosis, or assessment of prognosis of colorectal cancer in a subject. In some embodiments, such screening procedures may comprise fecal occult blood tests, sigmoidoscopy, barium enema X-ray, digital rectal exam, colonoscopy, detection of carcinoembryonic antigen (CEA) or combinations thereof.

In some embodiments, the polypeptides/polynucleotides are useful in assessing progression of colorectal pathogenesis. Such assessment may reflect the staging of the colorectal cancer. In some embodiments, the polypeptides/polynucleotides are useful in assessing or altering stage progression in a subject with colorectal cancer. When in reference to cancer staging, it is to be understood that any known means or classification system will apply, for any embodiment as described herein. In some embodiments, staging in reference to colorectal cancer may be via the Dukes' system and/or the International Union against Cancer-American Joint Committee on Cancer TNM staging system. Staging will reflect, in some embodiments, the extent of tumor penetration into the colon wall, with greater penetration generally correlating with a more dangerous tumor; the extent of invasion of the tumor through the colon wall and into other neighboring tissues, with greater invasion generally correlating with a more dangerous tumor; the extent of invasion of the tumor into the regional lymph nodes, and the extent of metastatic invasion into more distant tissues, such as the liver. It is to be understood that the polypeptides/polynucleotides of this invention may be useful both in the identification/assessment of colorectal cancer pathogenesis as a function of stage designation, as well as

In some embodiments, the polypeptides/polynucleotides of this invention may be useful in the diagnosis, treatment and/or assessment of prognosis of colon cancer. According to this aspect and in one embodiment, the polypeptides useful in this context are: T58132 or homologues thereof, or polynucleotides encoding the same. In some embodiments, these polypeptides/polynucleotides are used alone or in combination with one or more other polypeptides/polynucleotides of this invention, and/or in combination with other markers for colorectal cancer, including but not limited to CEA, CA19-9, CA50, and/or in combination with a native protein associated with the polypeptides of this invention, for example, native proteins of which the polypeptides are variants thereof. In some embodiments, the polypeptides/polynucleotides of this invention may be useful in, inter alia, assessing the presence, risk and/or extent of the following:
1. Early diagnosis, staging, grading, prognosis, monitoring, and treatment of diseases associated with colon cancer, or to indicate a predisposition to such for preventative measures.
2. The identification of a metastasis of unknown origin which originated from a primary colorectal cancer tumor, in particular when the metastasis is located in the liver, lung, bones, supraclavicluar lymph nodes or brain.
3. In the assessment of lymphadenopathy, in particular supraclavicluar or internal abdominal lymphadenopathy.
4. As a marker to distinguish between different types of colorectal tumors including but not limited to nonhereditary carcinoma, Familial Polyposis Coli, Hereditary nonpolyposis colon cancer (Lynch syndrome) and Carcinoid; therefore potentially affect treatment choice.
5. In the assessment of cancer staging, in addition and as a complementary measure to the Dukes system for staging colorectal cancer.
6. As a risk factor to the development of colorectal tumor, and in particular in diseases known to have high incidence of colorectal tumor, including but not limited to Crohn's disease and Ulcerative Colitis.
7. As a tool in the assessment of fecal occult blood or imaging findings suspected for colorectal tumor or abnormal blood tests associated with colorectal cancer including but not limited to elevated CEA level.
8. In the differential diagnosis between malignant and benign colorectal tumors, in particular adenomas and polyps.
9. Other conditions not mentioned above which have similar symptoms, signs and complications as colorectal cancer and where the differential diagnosis between them and colorectal cancer is of clinical importance including but not limited to:
   a. Any condition suggestive of a malignant tumor including but not limited to anorexia, cachexia, weight loss, fever, hypercalcemia, paraneoplastic syndrome.
   b. Lymphadenopathy, weight loss and other signs and symptoms associated with colorectal cancer but originate from diseases different from colorectal cancer including but not limited to other malignancies, infections and autoimmune diseases.
10. Prediction of patient's drug response
11. As surrogate markers for clinical outcome of a treated cancer.
   Renal Disease, and/or Condition

In one embodiment, the polypeptides and/or polynucleotides of this invention are utilized alone, or in combination with other markers, for the diagnosis, treatment selection and monitoring, or assessment of prognosis of any type of renal disease, and/or condition including but not limited to any type of renal damage; renal cancer, including but not limited to renal cell carcinoma; polycystic kidney disease; Diabetes induced nephropathy; Chronic Kidney Disease; Total Kidney Failure; Autoimmune nephropathy, including but not limited to Systemic lupus erythematosus (SLE), Goodpasture's syndrome, IgA nephropathy; Hereditary Nephritis—(Alport Syndrome); Infection-related Glomerular Disease, including but not limited to Acute post-streptococcal glomerulonephritis (PSGN), Bacterial endocarditis, HIV; Glomerulosclerosis; Focal segmental glomerulosclerosis (FSGS); Membranous nephropathy; Minimal change disease (MCD).

In some embodiments, the polypeptides/polynucleotides of this invention are utilized in conjunction with other screening procedures, as well as the use of other markers, for the diagnosis, or assessment of prognosis of renal disease and/or in a subject. In some embodiment, such markers and/or screening procedures may comprise urinary protein, creatinine or creatinine clearance. In another embodiments, the other markers may comprise markers used for the diagnosis or assessment of prognosis of renal cancer, specifically of renal cell carcinoma, including but not limited to vascular endothelial growth factor, interleukin-12, the soluble interleukin-2 receptor, intercellular adhesion molecule-1, human chorionic gonadotropin beta, insulin-like growth factor-1 receptor, Carbonic anhydrase 9 (CA 9), endostatin, Thymidine phosphorylase or combinations thereof.

In some embodiments, the polypeptides/polynucleotides of this invention may be useful in the diagnosis, treatment and/or assessment of prognosis of renal diseases and/or conditions. According to this aspect and in one embodiment, the polypeptides/polynucleotides useful in this context are: N31842, or homologues thereof. In some embodiments, these polypeptides/polynucleotides are used alone or in combination with one or more other polypeptides/polynucleotides of this invention, and/or in combination with other markers known to detect kidney-related disorders, and/or screening procedures, including, inter alia, urinary protein, creatinine or creatinine clearance, and/or a native protein associated with the polypeptides of this invention, for example, native proteins of which the polypeptides are variants thereof.

### Candidate Markers

This section relates to examples of sequences according to the present invention, including illustrative methods of selection thereof.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

The markers of the present invention were tested with regard to their expression in various cancerous and non-cancerous tissue samples. Unless otherwise noted, all experimental data relates to variants of the present invention, named according to the segment being tested (as expression was tested through RT-PCR as described). A description of the samples used in the ovarian cancer testing panel is provided in Table 1_{—}1 or Table 1_{—}5 below. A description of the samples used in the lung cancer testing panel is provided in Table 1_{—}2 or Table 1_{—}6 below. A description of the samples used in the breast cancer testing panel is provided in Table 1_{—}3 or 1_{—}7 below. A description of the samples used in the colon cancer testing panel is provided in Table 1_{—}4 and 1_{—}8 below. The keys for the tables 1_{—}5, 1_{—}6, 1_{—}7 and 1_{—}8 are listed in tables 1_{—}5_{—}1, 1_{—}6_{—}1, 1_{—}7_{—}1 and 1_{—}8_{—}1, respectively. A description of the samples used in the normal tissue panel is provided in Table 1_{—}9 and 1_{—}10 below. Tests were then performed as described in the “Materials and Experimental Procedures” section below.

TABLE 1_1
Tissue samples in ovarian cancer testing panel

| Sample name | Lot number | Source | Pathology | Grade | age |
|---|---|---|---|---|---|
| 33-B-Pap Sero CystAde G1 | A503175 | BioChain | Serous papillary cystadenocarcinoma | 1 | 41 |
| 41-G-Mix Sero/Muc/Endo G2 | 98-03-G803 | GOG | Mixed epithelial cystadenocarcinoma with mucinous, | 2 | 38 |
| | | | endometrioid, squamous and papillary serous (Stage2) | | |
| 35-G-Endo Adeno G2 | 94-08-7604 | GOG | Endometrioid adenocarcinoma | 2 | 39 |
| 14-B-Adeno G2 | A501111 | BioChain | Adenocarcinoma | 2 | 41 |
| 12-B-Adeno G3 | A406023 | Biochain | Adenocarcinoma | 3 | 45 |
| 40-G-Mix Sero/Endo G2 | 95-11-G006 | GOG | Papillary serous and endometrioid cystadenocarcinoma | 2 | 49 |
| | | | (Stage3C) | | |
| 4-A-Pap CystAdeno G2 | ILS-7286 | ABS | Papillary cystadenocarcinoma | 2 | 50 |
| 3-A-Pap Adeno G2 | ILS-1431 | ABS | Papillary adenocarcinoma | 2 | 52 |
| 2-A-Pap Adeno G2 | ILS-1408 | ABS | Papillary adenocarcinoma | 2 | 53 |
| 5-G-Adeno G3 | 99-12-G432 | GOG | Adenocarcinoma (Stage3C) | 3 | 46 |
| 11-B-Adeno G3 | A407068 | Biochain | Adenocarcinoma | 3 | 49 |
| 39--G-Mix Sero/Endo G3 | 2001-12-G037 | GOG | Mixed serous and endometrioid adenocarcinoma | 3 | 49 |
| 29-G-Sero Adeno G3 | 2001-12-G035 | GOG | Serous adenocarcinoma (Stage3A) | 3 | 50 |
| 70-G-Pap Sero Adeno G3 | 95-08-G069 | GOG | Papillary serous adenocarcinoma | 3 | 50 |
| 6-A-Adeno G3 | A0106 | ABS | adenocarcinoma | 3 | 51 |
| 31-B-Pap Sero CystAde G3 | A503176 | BioChain | Serous papillary cystadenocarcinoma | 3 | 52 |
| 25-A-Pap Sero Adeno G3 | N0021 | ABS | Papillary serous adenocarcinoma (StageT3CN1MX) | 3 | 55 |
| 37-G-Mix Sero/Endo G3 | 2002-05-G513 | GOG | Mixed serous and endometrioid adenocarcinoma | 3 | 56 |
| 7-A-Adeno G3 | IND-00375 | ABS | adenocarcinoma | 3 | 59 |
| 8-B-Adeno G3 | A501113 | BioChain | adenocarcinoma | 3 | 60 |
| 10-B-Adeno G3 | A407069 | Biochain | Adenocarcinoma | 3 | 60 |
| 38-G-Mix Sero/Endo G3 | 2002-05-G509 | GOG | Mixed serous and endometrioid adenocarcinoma of mullerian | 3 | 64 |
| | | | (Stage3C) | | |
| 13-G-Adeno G3 | 94-05-7603 | GOG | Poorly differentiated adenocarcinoma from primary peritoneal | 3 | 67 |
| 24-G-Pap Sero Adeno G3 | 2001-07-G801 | GOG | Papillary serous adenocarcinoma | 3 | 68 |
| 34-G-Pap Endo Adeno G3 | 95-04-2002 | GOG | Papillary endometrioid adenocarcinoma (Stage3C) | 3 | 68 |
| 30-G-Pap Sero Adeno G3 | 2001-08-G011 | GOG | Papillary serous carcinoma (Stage1C) | 3 | 72 |
| 1-A-Pap Adeno G3 | ILS-1406 | ABS | Papillary adenocarcinoma | 3 | 73 |
| 9-G-Adeno G3 | 99-06-G901 | GOG | Adenocarcinoma (maybe serous) | 3 | 84 |
| 32-G-Pap Sero CystAde G3 | 93-09-4901 | GOG | Serous papillary cystadenocarcinoma | 3 | 67 |
| 66-G-Pap Sero Adeno G3 SIV | 2000-01-G413 | GOG | Papillary serous carcinoma (metastais of primary | 3 | 67 |
| | | | peritoneum) (Stage4) | | |
| 19-B-Muc Adeno G3 | A504085 | BioChain | Mucinous adenocarcinoma | 3 | 34 |
| 21-G-Muc CystAde G2-3 | 95-10-G020 | GOG | Mucinous cystadenocarcinoma (Stage2) | 2-3 | 44 |
| 18-B-Muc Adeno G3 | A504083 | BioChain | Mucinous adenocarcinoma | 3 | 45 |
| 20-A-Pap Muc CystAde | USA-00273 | ABS | Papillary mucinous cystadenocarcinoma | | 46 |
| 17-B-Muc Adeno G3 | A504084 | BioChain | Mucinous adenocarcinoma | 3 | 51 |
| 22-A-Muc CystAde G2 | A0139 | ABS | Mucinous cystadenocarcinoma (Stage1C) | 2 | 72 |
| 43-G-Clear cell Adeno G3 | 2001-10-G002 | GOG | Clear cell adenocarcinoma | 3 | 74 |
| 44-G-Clear cell Adeno | 2001-07-G084 | GOG | Clear cell adenocarcinoma (Stage3A) | | 73 |
| 15-B-Adeno G3 | A407065 | BioChain | Carcinoma | 3 | 27 |
| 16-Ct-Adeno | 1090387 | Clontech | Carcinoma NOS | NA | 58 |
| 23-A-Muc CystAde G3 | VNM-00187 | ABS | Mucinous cystadenocarcinoma with low malignant | 3 | 45 |
| 42-G-Adeno borderline | 98-08-G001 | GOG | Epithelial adenocarcinoma of borderline malignancy | | 46 |
| 63-G-Sero CysAdenoFibroma | 2000-10-G620 | GOG | Serous CysAdenoFibroma of borderline malignancy | | 71 |
| 62-G-Ben Muc CysAdenoma | 99-10-G442 | GOG | Benbin mucinus cysadenoma | | 32 |
| 60-G-Muc CysAdenoma | 99-01-G043 | GOG | Mucinous Cysadenoma | | 40 |
| 56-G-Ben Muc CysAdeno | 99-01-G407 | GOG | Bengin mucinus cysadenoma | | 46 |
| 64-G-Ben Sero CysAdenoma | 99-06-G039 | GOG | Bengin Serous CysAdenoma | | 57 |
| 61-G-Muc CysAdenoma | 99-07-G011 | GOG | Mucinous Cysadenoma | | 63 |
| 59-G-Sero CysAdenoFibroma | 98-12-G401 | GOG | Serous CysAdenoFibroma | | 77 |
| 51-G-N M41 | 98-03-G803N | GOG | Normal (matched tumor 98-03-G803) | | 38 |
| 75-G-N M60 | 99-01-G043N | GOG | Normal (matched tumor 99-01-G043) | | 40 |
| 49-B-N M14 | A501112 | BioChain | Normal (matched tumor A501111) | | 41 |
| 52-G-N M42 | 98-08-G001N | GOG | Normal (matched tumor 98-08-G001) | | 46 |
| 68-G-N M56 | 99-01-G407N | GOG | Normal (matched bengin 99-01-G407) | | 46 |
| 50-B-N M8 | A501114 | BioChain | Normal (matched tumor A501113) | | 60 |
| 67-G-N M38 | 2002-05-509N | GOG | Normal (matched tumor 2002-05-G509) | | 64 |
| 69-G-N M24 | 2001-07- | GOG | Normal (matched tumor 2001-07-G801) | | 68 |
| | G801N | | | | |
| 73-G-N M59 | 98-12-G401N | GOG | Normal (matched tumor 98-12-G401) | | 77 |
| 72-G-N M66 | 2000-01- | GOG | Normal (matched tumor 2000-01-G413) | | |
| | G413N | | | | |
| 45-B-N | A503274 | BioChain | Normal PM | | 41 |
| 46-B-N | A504086 | BioChain | Normal PM | | 41 |
| 71-CG-N | CG-188-7 | Ichilov | Normal PM | | 49 |
| 48-B-N | A504087 | BioChain | Normal PM | | 51 |

TABLE 1_2
Tissue samples in lung cancer testing panel

| sample name | Lot No. | source | pathology | Grade | gender/age |
|---|---|---|---|---|---|
| 1-B-Adeno G1 | A504117 | Biochain | Adenocarcinoma | 1 | F/29 |
| 2-B-Adeno G1 | A504118 | Biochain | Adenocarcinoma | 1 | M/64 |
| 95-B-Adeno G1 | A610063 | Biochain | Adenocarcinoma | 1 | F/54 |
| 12-B-Adeno G2 | A504119 | Biochain | Adenocarcinoma | 2 | F/74 |
| 75-B-Adeno G2 | A609217 | Biochain | Adenocarcinoma | 2 | M/65 |
| 77-B-Adeno G2 | A608301 | Biochain | Adenocarcinoma | 2 | M/44 |
| 13-B-Adeno G2-3 | A504116 | Biochain | Adenocarcinoma | 2-3 | M/64 |
| 89-B-Adeno G2-3 | A609077 | Biochain | Adenocarcinoma | 2-3 | M/62 |
| 76-B-Adeno G3 | A609218 | Biochain | Adenocarcinoma | 3 | M/57 |
| 94-B-Adeno G3 | A610118 | Biochain | Adenocarcinoma | 3 | M/68 |
| 3-CG-Adeno | CG-200 | Ichilov | Adenocarcinoma | | NA |
| 14-CG-Adeno | CG-111 | Ichilov | Adenocarcinoma | | M/68 |
| 15-CG-Bronch adeno | CG-244 | Ichilov | Bronchioloalveolar adenocarcinoma | | M/74 |
| 45-B-Alvelous Adeno | A501221 | Biochain | Alveolus carcinoma | | F/50 |
| 44-B-Alvelous Adeno G2 | A501123 | Biochain | Alveolus carcinoma | 2 | F/61 |
| 19-B-Squamous G1 | A408175 | Biochain | Squamous carcinoma | 1 | M/78 |
| 16-B-Squamous G2 | A409091 | Biochain | Squamous carcinoma | 2 | F/68 |
| 17-B-Squamous G2 | A503183 | Biochain | Squamous carcinoma | 2 | M/57 |
| 21-B-Squamous G2 | A503187 | Biochain | Squamous carcinoma | 2 | M/52 |
| 78-B-Squamous G2 | A607125 | Biochain | Squamous Cell Carcinoma | 2 | M/62 |
| 80-B-Squamous G2 | A609163 | Biochain | Squamous Cell Carcinoma | 2 | M/74 |
| 18-B-Squamous G2-3 | A503387 | Biochain | Squamous Cell Carcinoma | 2-3 | M/63 |
| 81-B-Squamous G3 | A609076 | Biochain | Squamous Carcinoma | 3 | m/53 |
| 79-B-Squamous G3 | A609018 | Biochain | Squamous Cell Carcinoma | 3 | M/67 |
| 20-B-Squamous | A501121 | Biochain | Squamous Carcinoma | | M/64 |
| 22-B-Squamous | A503386 | Biochain | Squamous Carcinoma | | M/48 |
| 88-B-Squamous | A609219 | Biochain | Squamous Cell Carcinoma | | M/64 |
| 100-B-Squamous | A409017 | Biochain | Squamous Carcinoma | | M/64 |
| 23-CG-Squamous | CG-109 (1) | Ichilov | Squamous Carcinoma | | M/65 |
| 24-CG-Squamous | CG-123 | Ichilov | Squamous Carcinoma | | M/76 |
| 25-CG-Squamous | CG-204 | Ichilov | Squamous Carcinoma | | M/72 |
| 87-B-Large cell G3 | A609165 | Biochain | Large Cell Carcinoma | 3 | F/47 |
| 38-B-Large cell | A504113 | Biochain | Large cell | | M/58 |
| 39-B-Large cell | A504114 | Biochain | Large cell | | F/35 |
| 82-B-Large cell | A609170 | Biochain | Large Cell Neuroendocrine Carcinoma | | M/68 |
| 30-B-Small cell carci G3 | A501389 | Biochain | small cell | 3 | M/34 |
| 31-B-Small cell carci G3 | A501390 | Biochain | small cell | 3 | F/59 |
| 32-B-Small cell carci G3 | A501391 | Biochain | small cell | 3 | M/30 |
| 33-B-Small cell carci G3 | A504115 | Biochain | small cell | 3 | M |
| 86-B-Small cell carci G3 | A608032 | Biochain | Small Cell Carcinoma | 3 | F/52 |
| 83-B-Small cell carci | A609162 | Biochain | Small Cell Carcinoma | | F/47 |
| 84-B-Small cell carci | A609167 | Biochain | Small Cell Carcinoma | | F/59 |
| 85-B-Small cell carci | A609169 | Biochain | Small Cell Carcinoma | | M/66 |
| 46-B-N M44 | A501124 | Biochain | Normal M44 | | F/61 |
| 47-B-N | A503205 | Biochain | Normal PM | | M/26 |
| 48-B-N | A503206 | Biochain | Normal PM | | M/44 |
| 49-B-N | A503384 | Biochain | Normal PM | | M/27 |
| 50-B-N | A503385 | Biochain | Normal PM | | M/28 |
| 90-B-N | A608152 | Biochain | Normal (Pool 2) PM | | pool 2 |
| 91-B-N | A607257 | Biochain | Normal (Pool 2) PM | | pool 2 |
| 92-B-N | A503204 | Biochain | Normal PM | | m/28 |
| 93-Am-N | 111P0103A | Ambion | Normal PM | | F/61 |
| 96-Am-N | 36853 | Ambion | Normal PM | | F/43 |
| 97-Am-N | 36854 | Ambion | Normal PM | | M/46 |
| 98-Am-N | 36855 | Ambion | Normal PM | | F/72 |
| 99-Am-N | 36856 | Ambion | Normal PM | | M/31 |

TABLE 1_3
Tissue samples in breast cancer testing panel

| sample name | Lot no | source | pathology | grade | age | TNM | stage |
|---|---|---|---|---|---|---|---|
| 14-A-IDC G2 | A0135T | ABS | IDC | 2 | 37 | T2N2Mx | |
| 43-B-IDC G2 | A609183 | Biochain | IDC | 2 | 40 | | |
| 54-B-IDC G2 | A605353 | Biochain | IDC | 2 | 41 | | |
| 55-B-IDC G2 | A609179 | Biochain | IDC | 2 | 42 | | |
| 47-B-IDC G2 | A609221 | Biochain | IDC | 2 | 42 | | |
| 17-A-IDC G2 | 4904020036T | ABS | IDC | 2-3 | 42 | T3N1Mx | |
| 42-A-IDC G3 | 6005020031T | ABS | IDC | 3 | 42 | T1cN0Mx | |
| 7-A-IDC G2 | 7263T | ABS | IDC | 2 | 43 | T1N0M0 | stage 1 |
| 48-B-IDC G2 | A609222 | Biochain | IDC | 2 | 44 | | |
| 53-B-IDC G2 | A605151 | Biochain | IDC | 2 | 44 | | |
| 12-A-IDC G2 | 1432T | ABS | IDC | 2 | 46 | T2N0M0 | stage 2A |
| 61-B-IDC G2 | A610029 | Biochain | IDC | 2 | 46 | | |
| 46-B-Carci G2 | A609177 | Biochain | Carcinoma | 2 | 48 | | |
| 16-A-IDC G2 | 4904020032T | ABS | IDC | 2 | 49 | T3N1Mx | |
| 62-B-IDC G2 | A609194 | Biochain | IDC | 2 | 51 | | |
| 49-B-IDC G2 | A609223 | Biochain | IDC | 2 | 54 | | |
| 32-A-Muc Carci | 7116T | ABS | Mucinous carcinoma | | 54 | T2N0M0 | stage 2A |
| 45-B-IDC G2 | A609181 | Biochain | IDC | 2 | 58 | | |
| 15-A-IDC G2 | 7259T | ABS | IDC | 2 | 59 | T3N1M0 | stage 3A |
| 52-B-ILC G1 | A605360 | Biochain | Invasive Lobular Carcinoma | 1 | 60 | | |
| 6-A-IDC G1 | 7238T | ABS | IDC | 1 | 60 | T2N0M0 | stage 2A |
| 26-A-IDC G3 | 7249T | ABS | IDC | 3 | 60 | T2N0M0 | stage 2A |
| 13-A-IDC G2 | A0133T | ABS | IDC | 2 | 63 | T2N1aMx | |
| 50-B-IDC G2 | A609224 | Biochain | IDC | 2 | 69 | | |
| 44-B-IDC G2 | A609198 | Biochain | IDC | 2 | 77 | | |
| 51-B-IDC G1 | A605361 | Biochain | IDC | 1 | 79 | | |
| 31-CG-IDC | CG-154 | Ichilov | IDC | | 83 | | |
| 27-A-IDC G3 | 4907020072T | ABS | IDC | 3 | 91 | T2N0Mx | |
| 36-A-N M7 | 7263N | ABS | Normal matched to 7T | | 43 | | |
| 40-A-N M12 | 1432N | ABS | Normal matched to 12T | | 46 | | |
| 39-A-N M15 | 7259N | ABS | Normal matched to 15T | | 59 | | |
| 35-A-N M6 | 7238N | ABS | Normal matched to 6T | | 60 | | |
| 41-A-N M26 | 7249N | ABS | Normal matched to 26T | | 60 | | |
| 57-B-N | A609233 | Biochain | Normal PM | | 34 | | |
| 59-B-N | A607155 | Biochain | Normal PM | | 35 | | |
| 60-B-N | A609234 | Biochain | Normal PM | | 36 | | |
| 63-Am-N | 26486 | Ambion | Normal PS | | 43 | | |
| 66-Am-N | 36678 | Ambion | Normal PM | | 45 | | |
| 64-Am-N | 23036 | Ambion | Normal PM | | 57 | | |
| 56-B-N | A609235 | Biochain | Normal PM | | 59 | | |
| 65-Am-N | 31410 | Ambion | Normal PM | | 63 | | |
| 67-Am-N | 073P010602086A | Ambion | Normal PM | | 64 | | |
| 58-B-N | A609232 | Biochain | Normal PM | | 65 | | |

TABLE 1_4
Tissue samples in colon cancer testing panel

| sample name | Lot No. | tissue | source | pathology | Grade | gender/age |
|---|---|---|---|---|---|---|
| 58-B-Adeno G1 | A609152 | Colon | biochain | Adenocarcinoma | 1 | M/73 |
| 59-B-Adeno G1 | A609059 | Colon | biochain | Adenocarcinoma, Ulcer | 1 | M/58 |
| 14-CG-Polypoid Adeno G1 | CG-222 (2) | Rectum | Ichilov | Well polypoid adeocarcinoma Duke's C | | F/49 |
| D-C | | | | | | |
| 17-CG-Adeno G1-2 | CG-163 | Rectum | Ichilov | Adenocarcinoma | 2 | M/73 |
| 10-CG-Adeno G1-2 D-B2 | CG-311 | Sigmod colon | Ichilov | Adenocarcinoma Astler-Coller B2. | 1-2 | M/88 |
| 11-CG-Adeno G1-2 D-C2 | CG-337 | Colon | Ichilov | Adenocarcinoma Astler-Coller C2. | 1-2 | NA |
| 6-CG-Adeno G1-2 D-C2 | CG-303 (3) | Colon | Ichilov | Adenocarcinoma Astler-Coller C2. | 1-2 | F/77 |
| 5-CG-Adeno G2 | CG-308 | Colon Sigma | Ichilov | Adenocarcinoma. | 2 | F/80 |
| 16-CG-Adeno G2 | CG-278C | colon | Ichilov | Adenocarcinoma | 2 | F/60 |
| 56-B-Adeno G2 | A609148 | Colon | biochain | Adenocarcinoma | 2 | F48 |
| 61-B-Adeno G2 | A606258 | Colon | biochain | Adenocarcinoma, Ulcer | 2 | M/41 |
| 60-B-Adeno G2 | A609058 | Colon | biochain | Adenocarcinoma, Ulcer | 2 | M/67 |
| 22-CG-Adeno G2 D-B | CG-229C | Colon | Ichilov | Adenocarcinoma Duke's B | 2 | F/55 |
| 1-CG-Adeno G2 D-B2 | CG-335 | Cecum | Ichilov | Adenocarcinoma Dukes B2. | 2 | F/66 |
| 12-CG-Adeno G2 D-B2 | CG-340 | Colon Sigma | Ichilov | Adenocarcinoma Astler-Coller B2. | 2 | M/66 |
| 28-CG-Adeno G2 D-B2 | CG-284 | sigma | Ichilov | Adenocarcinoma Duke's B2 | 2 | F/72 |
| 2-CG-Adeno G2 D-C2 | CG-307 X2 | Cecum | Ichilov | Adenocarcinoma Astler-Coller C2. | 2 | F/89 |
| 9-CG-Adeno G2 D-D | CG-297 X2 | Rectum | Ichilov | Adenocarcinoma Dukes D. | 2 | M/62 |
| 13-CG-Adeno G2 D-D | CG-290 X2 | Rectosigmoidal | Ichilov | Adenocarcinoma Dukes D. | 2 | M/47 |
| | | colon | | | | |
| 26-CG-Adeno G2 D-D | CG-283 | sigma | Ichilov | Colonic adenocarcinoma Duke's D | 2 | F/63 |
| 4-CG-Adeno G3 | CG-276 | Colon | Ichilov | Carcinoma. | 3 | M/64 |
| 53-B-Adeno G3 | A609161 | Colon | biochain | Adenocarcinoma | 3 | F/53 |
| 54-B-Adeno G3 | A609142 | Colon | biochain | Adenocarcinoma | 3 | M/53 |
| 55-B-Adeno G3 | A609144 | Colon | biochain | Adenocarcinoma | 3 | M/68 |
| 57-B-Adeno G3 | A609150 | Colon | biochain | Adenocarcinoma | 3 | F/45 |
| 72-CG-Adeno G3 | CG-309 | colon | Ichilov | Adenocarcinoma | 3 | F/88 |
| 20-CG-Adeno G3 D-B2 | CG-249 | Colon | Ichilov | Ulcerated adenocarcinoma Duke's B2 | 3 | M/36 |
| 7-CG-Adeno D-A | CG-235 | Rectum | Ichilov | Adenocarcinoma intramucosal Duke's A. | | F/66 |
| 23-CG-Adeno D-C | CG-282 | sigma | Ichilov | Mucinus adenocarcinoma Astler Coller C | | M/51 |
| 3-CG-Muc adeno D-D | CG-224 | Colon | Ichilov | Mucinois adenocarcinoma Duke's D | | M/48 |
| 18-CG-Adeno | CG-22C | Colon | Ichilov | Adenocarcinoma | | NA |
| 19-CG-Adeno | CG-19C (1) | Colon | Ichilov | Adenocarcinoma | | NA |
| 21-CG-Adeno | CG-18C | Colon | Ichilov | Adenocarcinoma | | NA |
| 24-CG-Adeno | CG-12 (2) | Colon | Ichilov | Adenocarcinoma | | NA |
| 25-CG-Adeno | CG-2 | Colon | Ichilov | Adenocarcinoma | | NA |
| 27-CG-Adeno | CG-4 | Colon | Ichilov | Adenocarcinoma | | NA |
| 8-CG-diverticolosis, | CG-291 | Wall of sigma | Ichilov | Diverticolosis and diverticulitis of the Colon | | F/65 |
| diverticulitis | | | | | | |
| 46-CG-Crohn's disease | CG-338C | Cecum | Ichilov | Crohn's disease | | M/22 |
| 47-CG-Crohn's disease | CG-338AC | Colon | Ichilov | Crohn's disease. | | M/22 |
| 42-CG-N M20 | CG-249N | Colon | Ichilov | Normal | | M/36 |
| 43-CG-N M8 | CG-291N | Wall of sigma | Ichilov | Normal | | F/65 |
| 44-CG-N M21 | CG-18N | Colon | Ichilov | Normal | | NA |
| 45-CG-N M11 | CG-337N | Colon | Ichilov | Normal | | M/75 |
| 49-CG-N M14 | CG-222N | Rectum | Ichilov | Normal | | F/49 |
| 50-CG-N M5 | CG-308N | Sigma | Ichilov | Within normal limits | | F/80 |
| 51-CG-N M26 | CG-283N | Sigma | Ichilov | Normal | | F/63 |
| 41-B-N | A501156 | Colon | biochain | Normal PM | | M/78 |
| 52-CG-N | CG-309TR | Colon | Ichilov | Within normal limits | | F/88 |
| 62-B-N | A608273 | Colon | biochain | Normal PM | | M/66 |
| 63-B-N | A609260 | Colon | biochain | Normal PM | | M/61 |
| 64-B-N | A609261 | Colon | biochain | Normal PM | | F/68 |
| 65-B-N | A607115 | Colon | biochain | Normal PM | | M/24 |
| 66-B-N | A609262 | Colon | biochain | Normal PM | | M/58 |
| 67-B-N | A406029 | Colon | biochain | Normal PM (Pool of 10) | | |
| 69-B-N | A411078 | Colon | biochain | Normal PM (Pool of 10) | | F&M |
| 70-Cl-N | 1110101 | Colon | clontech | Normal PM (Pool of 3) | | |
| 71-Am-N | 071P10B | Colon | Ambion | Normal (IC BLEED) | | F/34 |

TABLE 1_5
Tissue samples in ovarian cancer testing panel

| sample_id | | | | | | | | | | |
|---|---|---|---|---|---|---|---|---|---|---|
| (GCI)/case | | | | | | | | | | |
| id | RNA | | | | | | | | | |
| (Asterand)/ | ID | | | | | | | | | |
| lot | (GCI)/ | | | | | | | | | |
| no. | Sample | | | | | | | | | |
| (old | ID | | C | | | Ethnic | | Menopausal | Mens | Preg |
| samples) | (Asterand) | Diag | Stage | Tumor % | age | BG | CA125PRE | Status | Age | Times |
| 23074 | 71900A2 | SA | I | 80 | 49 | CAU | | Pre-M | | 2 |
| 22653 | 70270A1 | SA | I | 90 | 69 | WCAU | | Post-M | | 1 |
| 18700 | 40771B1 | SA | IB | 100 | 62 | WCAU | | Post-M | | 3 |
| 93-09-4901 | | SPC | 1B | | 67 | | | | | |
| 17646 | 32667B1 | SA | IB | 100 | 68 | W | | Post-M | | 9 |
| 15644 | 22996A1 | SA | IC | 100 | 48 | CAU | | M | | 4 |
| 18701 | 40773C1 | SA | IIA | 100 | 59 | CAU | | Post-M | | 1 |
| 2O37O | | SA | IIB | 75 | 43 | WCAU | — | Pre-M | 12 | 0 |
| 7B3DP | | SA | IIB | 70 | 70 | WCAU | — | Post-M | 14 | 5 |
| 2001-08-G011 | | PSC | 3A | | 72 | | | | | |
| 95-08-G069 | | PSA | 3B | | 50 | | | | | |
| 99-12-G432 | | A | 3C | | 46 | | >500 | | | |
| 13268 | 19832A1 | SA | IIIC | 90 | 48 | C | | Post-M | | |
| 2001-12-G035 | | SA | 3C | | 50 | | 260 | | | |
| 3NTIS | | SA | IIIC | 70 | 53 | WCAU | 70 | Post-M | 12 | 1 |
| CEJUS | | SA | IIIC | 70 | 53 | WCAU | 4814 | Pre-M | — | 2 |
| 5NCLK | | SA | IIIC | 70 | 54 | WCAU | 209 | Post-M | 13 | 2 |
| N0021 | | PSA | 3C | | 55 | AU | | | | |
| 1HI5H | | SA | IIIC | 90 | 61 | WCAU | 34 | Post-M | 12 | 6 |
| 7RMHZ | | SA | IIIC | 80 | 63 | WCAU | — | Post-M | 12 | 2 |
| 4WAAB | | SA | IIIC | 90 | 63 | WCAU | — | Post-M | 11 | 2 |
| 79Z67 | | SA | IIIC | 85 | 67 | WCAU | — | Post-M | 12 | 6 |
| 94-05-7603 | | APP | 3C | | 67 | | | | | |
| DDSNL | | SA | IIIC | 70 | 68 | WCAU | — | Post-M | 11 | 4 |
| DH8PH | | SA | IV | 95 | 70 | WCAU | — | Post-M | 13 | 4 |
| A503175 | | SPC | | | 41 | Asian | | | | |
| A501111 | | A | | | 41 | Asian | | | | |
| A406023 | | A | | | 45 | Asian | | | | |
| A407068 | | A | | | 49 | Asian | | | | |
| ILS-7286 | | PC | UN | | 50 | Asian | | | | |
| A0106 | | A | UN | | 51 | Asian | | | | |
| ILS-1431 | | PA | UN | | 52 | Asian | | | | |
| A503176 | | SPC | | | 52 | Asian | | | | |
| ILS-1408 | | PA | UN | | 53 | Asian | | | | |
| IND-00375 | | A | | | 59 | Asian | | | | |
| A501113 | | A | | | 60 | Asian | | | | |
| A407069 | | A | | | 60 | Asian | | | | |
| ILS-1406 | | PA | UN | | 73 | Asian | | | | |
| E2WKF | | EA | IA | 70 | 30 | WCAU | — | Pre-M | 12 | 6 |
| 5895C | | EA | IA | 95 | 39 | WCAU | — | Pre-M | 14 | 2 |
| 533DX | | EA | IA | 95 | 50 | WCAU | 190 | Pre-M | 11 | 0 |
| HZ2EY | | EA | IA | 90 | 55 | WCAU | 1078 | Pre-M | 13 | 0 |
| RWOIV | | EA | IA | 65 | 47 | WCAU | 1695 | Pre-M | 14 | 0 |
| 1U52X | | EA | IIA | 95 | 61 | WCAU | 275 | | — | — |
| A17WS | | EA | IIB | 70 | 67 | WCAU | 78 | Post-M | 14 | 0 |
| 1VT3I | | EA | IIIC | 90 | 50 | WCAU | — | Pre-M | 12 | 2 |
| PZQXH | | EA | IIIC | 80 | 52 | WCAU | — | Pre-M | 11 | 0 |
| I8VHZ | | EA | IV | 90 | 68 | WCAU | — | Post-M | — | 2 |
| 98-03-G803 | | Mixed . . . | 2 | | 38 | | >35 | | | |
| 95- | | PS & | 3C | | 49 | | | | | |
| 11-G006 | | EC | | | | | | | | |
| 2002- | | MS & | 3C | | 56 | | | | | |
| 05-G513 | | EA | | | | | | | | |
| 2002- | | MS & | 3C | | 64 | | | | | |
| 05-G509 | | EAM | | | | | | | | |
| 95- | | PEA | 3C | | 68 | | | | | |
| 04-2002 | | | | | | | | | | |
| 95- | | MC | IA | | 44 | | | >100 | | |
| 10-G020 | | | | | | | | | | |
| IMDA1 | | MA | IC | 70 | 41 | WCAU | 50 | Pre-M | 12 | 2 |
| 12742 | 18920A1 | MA | IC | 70 | 61 | C | | Post-M | | 3 |
| A0139 | | MC | IC | | 72 | Asian | | | | |
| NJM4U | | MA | IIA | 80 | 51 | WCAU | | | | |
| USA- | | PMC | IIIA | | 45 | C | | | | |
| 00273 | | | | | | | | | | |
| RAFCW | | MA | IIIA | 75 | 55 | WCAU | 95 | Post-M | 13 | 4 |
| 23177 | 72888A1 | MA | IIIC | 60 | 52 | C | | Pre-M | | |
| 16103 | 29374B1 | MA | IIIC | 100 | 62 | W | | Post-M | | 1 |
| A504085 | | MA | | | 34 | Asian | | | | |
| A504083 | | MA | | | 45 | Asian | | | | |
| A504084 | | MA | | | 51 | Asian | | | | |
| A407065 | | C | | | 27 | Asian | | | | |
| 1090387 | | CNOS | | | 58 | Asian | | | | |
| 2001-07-G084 | | CCA | 1A | | 73 | | | | | |
| 2001- | | CCA | 3A | | 74 | | Slightly | | | |
| 10-G002 | | | | | | | elevateed | | | |
| SC656 | | MBT | IA | 75 | 40 | WCAU | 138 | Pre-M | 13 | 2 |
| 3D5FO | | MBT | IA | 85 | 51 | WCAU | 19 | ? | 15 | 0 |
| 7JP3F | | MBT | IA | 75 | 56 | WCAU | 125 | Post-M | 14 | 3 |
| VNM- | | MC | | | 45 | Asian | | | | |
| 00187 | | Low M | | | | | | | | |
| 98- | | EA | 1A | | 46 | | | | | |
| 08- | | of | | | | | | | | |
| G001 | | BM | | | | | | | | |
| 99- | | BMC | | | 32 | | 6 | | | |
| 10-G442 | | | | | | | | | | |
| QLIKY | | BMC | | 100 | 42 | WCAU | | | | |
| 16870 | 30534A1 | BMC | | 100 | 45 | W | | Pre-M | | 2 |
| 99- | | BMC | | | 46 | | | | | |
| 01-G407 | | | | | | | | | | |
| 943EC | | BMC | | 75 | 54 | WCAU | | | | |
| JO8W7 | | BMC | | 50 | 56 | WCAU | | | | |
| 17016 | 30645B1 | BSC | IA | 100 | 38 | C | | Pre-M | | 2 |
| 99- | | BSC | | | 57 | | | | | |
| 06-G039 | | | | | | | | | | |
| DQQ2F | | BSCF | | 95 | 68 | WCAU | | | | |
| 8786 | 8275A1 | BSC | | 100 | 80 | CAU | | Post-M | | 10 |
| 15690 | 23054A1 | NO- | | | 52 | CAU | | Pre-M | | 10 |
| | | BM | | | | | | | | |
| 16843 | 30488A1 | NO- | | | 57 | W | | Post-M | | 4 |
| | | BM | | | | | | | | |
| 16850 | 30496B1 | NO- | | | 65 | W | | Post-M | | 2 |
| | | BM | | | | | | | | |
| 16848 | 30499C1 | NO- | | | 66 | CAU | | Post-M | | 9 |
| | | BM | | | | | | | | |
| WPU1U | | NO- | | 0 | 32 | WC | | | | |
| | | PS | | | | | | | | |
| Y9VHI | | NO- | | 0 | 35 | WCAU | | | | |
| | | PS | | | | | | | | |
| 76VM9 | | NO- | | 0 | 41 | WCAU | | | | |
| | | PS | | | | | | | | |
| DWHTZ | | NO- | | 0 | 42 | WCAU | | | | |
| | | PS | | | | | | | | |
| SJ2R2 | | NO- | | 0 | 43 | WCAU | | | | |
| | | PS | | | | | | | | |
| 9RQMN | | NO- | | 0 | 45 | WCAU | | | | |
| | | PS | | | | | | | | |
| TOAE5 | | NO- | | 0 | 45 | WCAU | | | | |
| | | PS | | | | | | | | |
| TW9PM | | NO- | | 0 | 46 | WCAU | | | | |
| | | PS | | | | | | | | |
| 2VND2 | | NO- | | 0 | 46 | WCAU | | | | |
| | | PS | | | | | | | | |
| L629F | | NO- | | 0 | 47 | WCAU | | | | |
| | | PS | | | | | | | | |
| XLB23 | | NO- | | 0 | 47 | WCAU | | | | |
| | | PS | | | | | | | | |
| IDUVY | | NO- | | 0 | 47 | WCAU | | | | |
| | | PS | | | | | | | | |
| ZCXAD | | NO- | | 0 | 48 | WCAU | | | | |
| | | PS | | | | | | | | |
| PEQ6C | | NO- | | 0 | 49 | WCAU | | | | |
| | | PS | | | | | | | | |
| DD73B | | NO- | | 0 | 49 | WCAU | | | | |
| | | PS | | | | | | | | |
| E2UF7 | | NO- | | 0 | 53 | WCAU | | | | |
| | | PS | | | | | | | | |
| GWXUZ | | NO- | | 0 | 53 | WCAU | | | | |
| | | PS | | | | | | | | |
| 4YG5P | | NO- | | 0 | 55 | WCAU | | | | |
| | | PS | | | | | | | | |
| FDPL9 | | NO- | | 0 | 56 | WCAU | | | | |
| | | PS | | | | | | | | |
| A503274 | | NO- | | | 41 | Asian | | | | |
| | | PM | | | | | | | | |
| A504086 | | NO- | | | 41 | Asian | | | | |
| | | PM | | | | | | | | |
| CG- | | NO- | | | 49 | | | | | |
| 188-7 | | PM | | | | | | | | |
| A504087 | | NO- | | | 51 | Asian | | | | |
| | | PM | | | | | | | | |

| sample_id | | | | | | | | | | |
|---|---|---|---|---|---|---|---|---|---|---|
| (GCI)/case | | | | | | | | | | |
| id | | | | | | | | | | |
| (Asterand)/ | | | | | | | | | | |
| lot | | Age | | | | | | | | |
| no. | | at | | Oral | Oral | | | | | |
| (old | Preg | first | | Con | Con | Tubal | WT | HT | | Recovery |
| samples) | Toterm | child | OC | Length | Unit | ligation | (KG) | (CM) | BMI | Type |
| 23074 | 1 | | | | | | 82 | 169 | 28.7 | Surg |
| 22653 | 1 | | | | | | 80 | 167 | 28.7 | Surg |
| 18700 | 3 | | | | | | 72 | 160 | 28.1 | Surg |
| 93-09-4901 | | | | | | | | | | |
| 17646 | 2 | | | | | | 76 | 164 | 28.3 | Surg |
| 15644 | 2 | | | | | | 62 | 167 | 22 | Surg |
| 18701 | 1 | | | | | | 72 | 167 | 25.8 | Surg |
| 2O37O | 0 | 0 | NO | — | | NO | 90.7 | 168 | 32.3 | Surg |
| 7B3DP | 3 | 20 | YES | 6 | months | NO | 77.1 | 168 | 27.4 | Surg |
| 2001-08-G011 | | | | | | | | | | |
| 95-08-G069 | | | | | | | | | | |
| 99-12-G432 | | | | | | | | | | |
| 13268 | | | | | | | 72 | 168 | 25.5 | Surg |
| 2001-12-G035 | | | | | | | | | | |
| 3NTIS | 1 | 26 | YES | 3 | months | NO | 93.4 | 175 | 30.4 | Surg |
| CEJUS | 2 | 30 | NO | — | | NO | 88 | 165 | 32.3 | Surg |
| 5NCLK | 2 | 21 | YES | 1 | years | NO | 71.7 | 170 | 24.7 | Surg |
| N0021 | | | | | | | | | | |
| 1HI5H | 3 | 22 | NO | — | | NO | 99.8 | 160 | 39 | Surg |
| 7RMHZ | 2 | 20 | YES | 10 | years | NO | 68 | 165 | 25 | Surg |
| 4WAAB | 1 | 29 | YES | 4 | years | NO | 51 | 166 | 18.5 | Surg |
| 79Z67 | 5 | 24 | YES | 2 | years | YES | 76.2 | 163 | 28.8 | Surg |
| 94-05-7603 | | | | | | | | | | |
| DDSNL | 4 | 19 | NO | — | | NO | 59 | 155 | 24.6 | Surg |
| DH8PH | 3 | 20 | NO | — | | NO | 69.4 | 152 | 29.9 | Surg |
| A503175 | | | | | | | | | | |
| A501111 | | | | | | | | | | |
| A406023 | | | | | | | | | | |
| A407068 | | | | | | | | | | |
| ILS-7286 | | | | | | | | | | |
| A0106 | | | | | | | | | | |
| ILS-1431 | | | | | | | | | | |
| A503176 | | | | | | | | | | |
| ILS-1408 | | | | | | | | | | |
| IND-00375 | | | | | | | | | | |
| A501113 | | | | | | | | | | |
| A407069 | | | | | | | | | | |
| ILS-1406 | | | | | | | | | | |
| E2WKF | 5 | 17 | YES | 6 | years | NO | 92.5 | 168 | 32.9 | Surg |
| 5895C | 2 | 20 | NO | — | | NO | 86.2 | 170 | 29.8 | Surg |
| 533DX | — | — | YES | 2 | years | NO | 108 | 170 | 37.3 | Surg |
| HZ2EY | — | — | NO | — | | NO | 62 | 165 | 22.7 | Surg |
| RWOIV | — | — | NO | — | | NO | 105 | 168 | 37.4 | Surg |
| 1U52X | — | — | | — | | | 64.9 | 152 | 27.9 | Surg |
| A17WS | — | — | NO | — | | NO | 70.3 | 175 | 22.9 | Surg |
| 1VT3I | 2 | 24 | YES | 1 | years | NO | 59.9 | 155 | 24.9 | Surg |
| PZQXH | — | — | YES | 5 | years | NO | 57.6 | 163 | 21.8 | Surg |
| I8VHZ | 2 | 27 | NO | — | | NO | 73 | 160 | 28.5 | Surg |
| 98-03-G803 | | | | | | | | | | |
| 95- | | | | | | | | | | |
| 11-G006 | | | | | | | | | | |
| 2002- | | | | | | | | | | |
| 05-G513 | | | | | | | | | | |
| 2002- | | | | | | | | | | |
| 05-G509 | | | | | | | | | | |
| 95- | | | | | | | | | | |
| 04-2002 | | | | | | | | | | |
| 95- | | | | | | | | | | |
| 10-G020 | | | | | | | | | | |
| IMDA1 | 1 | 24 | NO | — | | | 85.3 | 168 | 30.3 | Surg |
| 12742 | 3 | | | | | | 70 | 155 | 29.1 | Surg |
| A0139 | | | | | | | | | | |
| NJM4U | | | | | | | 73.5 | 168 | 26.1 | Surg |
| USA- | | | | | | | | | | |
| 00273 | | | | | | | | | | |
| RAFCW | 3 | 22 | NO | — | | NO | 98 | 170 | 33.8 | Surg |
| 23177 | | | | | | | 100 | 162 | 38.1 | Surg |
| 16103 | 1 | | | | | | 40 | 160 | 15.6 | Surg |
| A504085 | | | | | | | | | | |
| A504083 | | | | | | | | | | |
| A504084 | | | | | | | | | | |
| A407065 | | | | | | | | | | |
| 1090387 | | | | | | | | | | |
| 2001-07-G084 | | | | | | | | | | |
| 2001- | | | | | | | | | | |
| 10-G002 | | | | | | | | | | |
| SC656 | 2 | 23 | NO | — | | YES | 83.9 | 160 | 32.8 | Surg |
| 3D5FO | — | — | NO | — | | NO | 79 | 163 | 29.9 | Surg |
| 7JP3F | 3 | 19 | YES | 5 | years | NO | 58 | 152 | 25.1 | Surg |
| VNM- | | | | | | | | | | |
| 00187 | | | | | | | | | | |
| 98- | | | | | | | | | | |
| 08- | | | | | | | | | | |
| G001 | | | | | | | | | | |
| 99- | | | | | | | | | | |
| 10-G442 | | | | | | | | | | |
| QLIKY | | | | | | | 72.6 | 175 | 23.6 | Surg |
| 16870 | 2 | | | | | | 100 | 169 | 35 | Surg |
| 99- | | | | | | | | | | |
| 01-G407 | | | | | | | | | | |
| 943EC | | | | | | | 170 | 168 | 60.5 | Surg |
| JO8W7 | | | | | | | 71.2 | 165 | 26.1 | Surg |
| 17016 | 2 | | | | | | 64 | 172 | 21.6 | Surg |
| 99- | | | | | | | | | | |
| 06-G039 | | | | | | | | | | |
| DQQ2F | | | | | | | 107 | 155 | 44.5 | Surg |
| 8786 | 9 | | | | | | 0 | 0 | 0 | Surg |
| 15690 | 3 | | | | | | 97 | 167 | 34.8 | Surg |
| 16843 | 2 | | | | | | 76 | 154 | 32 | Surg |
| 16850 | 2 | | | | | | 89 | 162 | 33.9 | Surg |
| 16848 | 2 | | | | | | 76 | 158 | 30.4 | Surg |
| WPU1U | | | | | | | 74.4 | 163 | 28.2 | Surg |
| Y9VHI | | | | | | | 81 | 170 | 28 | Surg |
| 76VM9 | | | | | | | 59 | 165 | 21.6 | Surg |
| DWHTZ | | | | | | | 92.5 | 168 | 32.9 | Surg |
| SJ2R2 | | | | | | | 65.8 | 155 | 27.4 | Surg |
| 9RQMN | | | | | | | 59 | 163 | 22.3 | Surg |
| TOAE5 | | | | | | | 87 | 165 | 31.9 | Surg |
| TW9PM | | | | | | | 70 | 173 | 23.5 | Surg |
| 2VND2 | | | | | | | 88.5 | 168 | 31.5 | Surg |
| L629F | | | | | | | 68 | 175 | 22.2 | Surg |
| XLB23 | | | | | | | 65 | 175 | 21.2 | Surg |
| IDUVY | | | | | | | 63 | 165 | 23.1 | Surg |
| ZCXAD | | | | | | | 102 | 163 | 38.6 | Surg |
| PEQ6C | | | | | | | 81.2 | 165 | 29.8 | Surg |
| DD73B | | | | | | | 88 | 163 | 33.3 | Surg |
| E2UF7 | | | | | | | 70.3 | 163 | 26.6 | Surg |
| GWXUZ | | | | | | | 72.6 | 165 | 26.6 | Surg |
| 4YG5P | | | | | | | 71.7 | 165 | 26.3 | Surg |
| FDPL9 | | | | | | | 82.1 | 167 | 29.4 | Surg |
| A503274 | | | | | | | | | | |
| A504086 | | | | | | | | | | |
| CG- | | | | | | | | | | |
| 188-7 | | | | | | | | | | |
| A504087 | | | | | | | | | | |

TABLE 1_5_1

| Key | Full Name |
|---|---|
| A | Adenocarcinoma |
| APP | Adenocarcinoma from primary peritoneal |
| BMC | BENIGN MUCINOUS CYSTADENOMA |
| BSC | BENIGN SEROUS CYSTADENOMA |
| BSCF | BENIGN SEROUS CYSTADENOFIBROMA |
| C | Carcinoma |
| C Stage | Cancer stage |
| CAU | Caucasian |
| CCA | Clear cell adenocarcinoma |
| CNOS | Carcinoma NOS |
| EA | ENDOMETROID ADENOCARCINOMA |
| EA of BM | Endometroid adenocarcinoma of borderline |
| | malignancy |
| HT | Height |
| M | Menopausal |
| MA | MUCINOUS ADENOCARCINOMA |
| MBT | MUCINOUS BORDERLINE TUMOR |
| MC | Mucinous cystadenocarcinoma |
| MC Low M | Mucinous cystadenocarcinoma with low malignant |
| Mens. Age | Menstrual Age |
| Mixed . . . | Mixed epithelial cystadenocarcinoma with mucinous, |
| | endometrioid, squamous and papillary serous |
| MS & EA | Mixed serous and endometrioid adenocarcinoma |
| MS & EAM | Mixed serous and endometrioid adenocarcinoma of |
| | mullerian |
| NO-BM | NORMAL OVARY-BM |
| NO-PM | NORMAL OVARY-PM |
| NO-PS | NORMAL OVARY-PS |
| OC | Oral Contraceptive |
| PA | Papillary adenocarcinoma |
| PC | Papillary cystadenocarcinoma |
| PEA | Papillary endometrioid adenocarcinoma |
| PMC | Papillary mucinous cystadenocarcinoma |
| Post-M | Post-menopausal |
| Pre-M | Pre-menopausal |
| PS & EC | Papillary serous and endometrioid cystadenocarcinoma |
| PSA | Papillary serous adenocarcinoma |
| PSC | Papillary serous carcinoma |
| SA | SEROUS ADENOCARCINOMA |
| SPC | Serous papillary cystadenocarcinoma |
| W | White |
| WCAU | WHITE/CAUCASIAN |
| WT | Weight |

TABLE 1_6
Tissue samples in lung cancer testing panel

| | | | sample | | | | | | | | | | |
|---|---|---|---|---|---|---|---|---|---|---|---|---|---|
| | | | id | | | | | | | | | | |
| | | | (GCI)/ | | | | | | | | | | |
| | | | case id | TISSUE | | | | | | | | | |
| | | | (Asterand)/ | ID | RNA | | | | | | | | |
| | | | lot | (GCI)/ | ID (GCI)/ | | | | | | | | |
| | | | no. | specimen | Sample | | | Speci- | | | | | |
| | Source/ | sample | (old | ID | ID | | Diag | men | | | | | |
| Tissue | Delivery | name | samples) | (Asterand) | (Asterand) | Diag | remarks | location | Gr | TNM | CS | Tum % | Gen |
| LC | GCI | 1-GC- | 7Z9V4 | 7Z9V4AYM | | Aden | BC | | | | IA | 80 | F |
| | | BAC-SIA | | | | | | | | | | | |
| LC | GCI | 2-GC- | ZW2AQ | ZW2AQARP | | Aden | BC | | | | IB | 70 | F |
| | | BAC-SIB | | | | | | | | | | | |
| LC | Bioch | 72-(44)- | A501123 | | | AC | | | 2 | UN | | | F |
| | | Bc-BAC | | | | | | | | | | | |
| LC | Bioch | 73-(45)- | A501221 | | | AC | | | UN | UN | | | F |
| | | Bc-BAC | | | | | | | | | | | |
| LC | GCI | 4-GC- | 3MOPL | 3MOPLA79 | | Aden | | | | | IA | 60 | M |
| | | Adeno- | | | | | | | | | | | |
| | | SIA | | | | | | | | | | | |
| LC | GCI | 5-GC- | KOJXD | KOJXDAV4 | | Aden | | | | | IA | 90 | F |
| | | Adeno- | | | | | | | | | | | |
| | | SIA | | | | | | | | | | | |
| LC | GCI | 6-GC- | X2Q44 | X2Q44A79 | | Aden | | | | | IA | 85 | M |
| | | Adeno- | | | | | | | | | | | |
| | | SIA | | | | | | | | | | | |
| LC | GCI | 7-GC- | 6BACZ | 6BACZAP5 | | Aden | | | | | IA | 60 | F |
| | | Adeno- | | | | | | | | | | | |
| | | SIA | | | | | | | | | | | |
| LC | GCI | 8-GC- | BS9AF | BS9AFA3E | | Aden | | | | | IA | 55 | F |
| | | Adeno- | | | | | | | | | | | |
| | | SIA | | | | | | | | | | | |
| LC | GCI | 9-GC- | UCLOA | UCLOAA9L | | Aden | | | | | IA | 80 | F |
| | | Adeno- | | | | | | | | | | | |
| | | SIA | | | | | | | | | | | |
| LC | GCI | 10-GC- | BVYK3 | BVYK3A7Z | | Aden | | | | | IA | 60 | F |
| | | Adeno- | | | | | | | | | | | |
| | | SIA | | | | | | | | | | | |
| LC | GCI | 11-GC- | U4DM4 | U4DM4AFZ | | Aden | | | | | IB | 65 | F |
| | | Adeno- | | | | | | | | | | | |
| | | SIB | | | | | | | | | | | |
| LC | GCI | 12-GC- | OWX5Y | OWX5YA3S | | Aden | | | | | IB | 90 | M |
| | | Adeno- | | | | | | | | | | | |
| | | SIB | | | | | | | | | | | |
| LC | GCI | 13-GC- | XYY96 | XYY96A6B | | Aden | | | | | IIA | 70 | F |
| | | Adeno- | | | | | | | | | | | |
| | | SIIA | | | | | | | | | | | |
| LC | GCI | 14-GC- | SO7B1 | SO7B1AIJ | | Aden | | | | | IIA | 70 | M |
| | | Adeno- | | | | | | | | | | | |
| | | SIIA | | | | | | | | | | | |
| LC | GCI | 15-GC- | QANSY | QANSYACD | | Aden | | | | | IIIA | 65 | F |
| | | Adeno- | | | | | | | | | | | |
| | | SIIIA | | | | | | | | | | | |
| LC | Bioch | 16-(95)- | A610063 | | | Aden | | | 1 | UN | | | F |
| | | BC- | | | | | | | | | | | |
| | | Adeno | | | | | | | | | | | |
| LC | Bioch | 17-(89)- | A609077 | | | Aden | | | 2-3 | UN | | | M |
| | | Bc- | | | | | | | | | | | |
| | | Adeno | | | | | | | | | | | |
| LC | Bioch | 18-(76)- | A609218 | | | Aden | | | 3 | UN | | | M |
| | | Bc- | | | | | | | | | | | |
| | | Adeno | | | | | | | | | | | |
| LC | Bioch | 74-(2)-Bc- | A504118 | | | Aden | | | 1 | UN | | | M |
| | | Adeno | | | | | | | | | | | |
| LC | Bioch | 76-(75)- | A609217 | | | Aden | | | 2 | UN | | | M |
| | | Bc- | | | | | | | | | | | |
| | | Adeno | | | | | | | | | | | |
| LC | Bioch | 77-(12)- | A504119 | | | Aden | | | 2 | UN | | | F |
| | | Bc- | | | | | | | | | | | |
| | | Adeno | | | | | | | | | | | |
| LC | Bioch | 78-(13)- | A504116 | | | Aden | | | 2-3 | UN | | | M |
| | | Bc- | | | | | | | | | | | |
| | | Adeno | | | | | | | | | | | |
| LC | Bioch | 79-(94)- | A610118 | | | Aden | | | 3 | UN | | | M |
| | | Bc- | | | | | | | | | | | |
| | | Adeno | | | | | | | | | | | |
| LC | Ichilov | 80-(3)-Ic- | CG-200 | | | Aden | | | UN | UN | | | F |
| | | Adeno | | | | | | | | | | | |
| LC | Ichilov | 81-(14)- | CG-111 | | | Aden | | | UN | UN | | | M |
| | | Ic-Adeno | | | | | | | | | | | |
| LC | Aster | 19-As-Sq- | 9220 | 9418 | 9418A1 | SCC | | | 1 | TXN0M0 | Occult | 80 | M |
| | | S0 | | | | | | | | | | | |
| LC | GCI | 20-GC- | U2QHS | U2QHSA2N | | SCC | | | | | IA | 55 | F |
| | | Sq-SIA | | | | | | | | | | | |
| LC | GCI | 21-GC- | TRQR7 | TRQR7ACD | | SCC | | | | | IB | 75 | M |
| | | Sq-SIB | | | | | | | | | | | |
| LC | Aster | 22-As-Sq- | 17581 | 32603 | 32603B1 | SCC | | | 3 | T2N0M0 | IB | 90 | M |
| | | SIB | | | | | | | | | | | |
| LC | Aster | 23-As-Sq- | 18309 | 41454 | 41454B1 | SCC | | | 2 | T2N0MX | IB | 100 | M |
| | | SIB | | | | | | | | | | | |
| LC | Aster | 24-As-Sq- | 9217 | 9415 | 9415B1 | SCC | | | 2 | T2N0M0 | IB | 90 | M |
| | | SIB | | | | | | | | | | | |
| LC | GCI | 25-GC- | RXQ1P | RXQ1PAEA | | SCC | | | | | IIB | 55 | F |
| | | Sq-SIIB | | | | | | | | | | | |
| LC | GCI | 26-GC- | KB5KH | KB5KHA6X | | SCC | | | | | IIB | 65 | M |
| | | Sq-SIIB | | | | | | | | | | | |
| LC | GCI | 27-GC- | LAYMB | LAYMBALF | | SCC | | | | | IIIA | 65 | F |
| | | Sq-SIIIA | | | | | | | | | | | |
| LC | Ichilov | 28-(23)- | CG-109 (1) | | | SCC | | | UN | UN | | | M |
| | | Ic-Sq | | | | | | | | | | | |
| LC | Ichilov | 29-(25)- | CG-204 | | | SCC | | | UN | UN | | | M |
| | | Ic-Sq | | | | | | | | | | | |
| LC | Bioch | 30-(19)- | A408175 | | | SCC | | | 1 | UN | | | M |
| | | Bc-Sq | | | | | | | | | | | |
| LC | Bioch | 31-(78)- | A607125 | | | SCC | | | 2 | UN | | | M |
| | | Bc-Sq | | | | | | | | | | | |
| LC | Bioch | 32-(16)- | A409091 | | | SCC | | | 2 | UN | | | F |
| | | Bc-Sq | | | | | | | | | | | |
| LC | Bioch | 33-(80)- | A609163 | | | SCC | | | 2 | UN | | | M |
| | | Bc-Sq | | | | | | | | | | | |
| LC | Bioch | 34-(18)- | A503387 | | | SCC | | | 2-3 | UN | | | M |
| | | Bc-Sq | | | | | | | | | | | |
| LC | Bioch | 92-(38)- | A504113 | | | LCC | | | UN | UN | | | M |
| | | Bc-LCC | | | | | | | | | | | |
| LC | Bioch | 93-(82)- | A609170 | | | LCNC | | | UN | UN | | | M |
| | | Bc-LCC | | | | | | | | | | | |
| LC | GCI | 42-GC- | QPJQL | QPJQLAF6 | | SMCC | NC | | 3 | | IB | 65 | F |
| | | SCC-SIB | | | | | | | | | | | |
| LC | Bioch | 43-(32)- | A501391 | | | SMCC | | | | UN | | | M |
| | | Bc-SCC | | | | | | | | | | | |
| LC | Bioch | 44-(30)- | A501389 | | | SMCC | | | 3 | UN | | | M |
| | | Bc-SCC | | | | | | | | | | | |
| LC | Bioch | 45-(83)- | A609162 | | | SMCC | | | UN | UN | | | F |
| | | Bc-SCC | | | | | | | | | | | |
| LC | Bioch | 46-(86)- | A608032 | | | SMCC | | | 3 | UN | | | F |
| | | Bc-SCC | | | | | | | | | | | |
| LC | Bioch | 47-(31)- | A501390 | | | SMCC | | | | UN | | | F |
| | | Bc-SCC | | | | | | | | | | | |
| LC | Bioch | 48-(84)- | A609167 | | | SMCC | | | UN | UN | | | F |
| | | Bc-SCC | | | | | | | | | | | |
| LC | Bioch | 49-(85)- | A609169 | | | SMCC | | | UN | UN | | | M |
| | | Bc-SCC | | | | | | | | | | | |
| LC | Bioch | 50-(33)- | A504115 | | | SMCC | | | | UN | | | M |
| | | Bc-SCC | | | | | | | | | | | |
| LN | Aster | 51-As-N- | 9078 | 9275 | 9275B1 | Norm-L | PS | | | | | | M |
| | | PS | | | | | | | | | | | |
| LN | Aster | 52-As-N- | 8757 | 8100 | 8100B1 | Norm-L | PM | (Right), | | | | | F |
| | | PM | | | | | | Lobe | | | | | |
| | | | | | | | | Inferior | | | | | |
| LN | Aster | 53-As-N- | 6692 | 6161 | 6161A1 | Norm-L | PM | | | | | | M |
| | | PM | | | | | | | | | | | |
| LN | Aster | 54-As-N- | 7900 | 7180 | 7180F1 | Norm-L | PM | | | | | | F |
| | | PM | | | | | | | | | | | |
| LN | Aster | 55-As-N- | 8771 | 8163 | 8163A1 | Norm-L | PM | (Left), | | | | | M |
| | | PM | | | | | | Lobe | | | | | |
| | | | | | | | | Supe- | | | | | |
| | | | | | | | | rior | | | | | |
| LC | Bioch | 35-(81)- | A609076 | | | SCC | | | 3 | UN | | | M |
| | | Bc-Sq | | | | | | | | | | | |
| LC | Bioch | 82-(21)- | A503187 | | | SCC | | | 2 | UN | | | M |
| | | Bc-Sq | | | | | | | | | | | |
| LC | Bioch | 83-(17)- | A503183 | | | SCC | | | 2 | UN | | | M |
| | | Bc-Sq | | | | | | | | | | | |
| LC | Bioch | 84-(79)- | A609018 | | | SCC | | | 3 | UN | | | M |
| | | Bc-Sq | | | | | | | | | | | |
| LC | Bioch | 85-(22)- | A503386 | | | SCC | | | UN | UN | | | M |
| | | Bc-Sq | | | | | | | | | | | |
| LC | Bioch | 86-(20)- | A501121 | | | SCC | | | UN | UN | | | M |
| | | Bc-Sq | | | | | | | | | | | |
| LC | Bioch | 87-(88)- | A609219 | | | SCC | | | UN | UN | | | M |
| | | Bc-Sq | | | | | | | | | | | |
| LC | Bioch | 88-(100)- | A409017 | | | SCC | | | UN | UN | | | M |
| | | Bc-Sq | | | | | | | | | | | |
| LC | Ichilov | 89-(24)- | CG-123 | | | SCC | | | UN | UN | | | M |
| | | Ic-Sq | | | | | | | | | | | |
| LC | GCI | 36-GC- | AF8AL | AF8ALAAL | | LCC | | | | | IA | 85 | M |
| | | LCC-SIA | | | | | | | | | | | |
| LC | GCI | 37-GC- | O62XU | O62XUA1X | | LCC | | | | | IB | 75 | F |
| | | LCC-SIB | | | | | | | | | | | |
| LC | GCI | 38-GC- | OLOIM | OLOIMAS1 | | LCC | | | | | IB | 70 | M |
| | | LCC-SIB | | | | | | | | | | | |
| LC | GCI | 39-GC- | 1ZWSV | 1ZWSVAB9 | | LCC | | | | | IIB | 50 | M |
| | | LCC-SIIB | | | | | | | | | | | |
| LC | GCI | 40-GC- | 2YHOD | 2YHODA1H | | LCC | NSCC | | | | IIB | 95 | M |
| | | LCC-SIIB | | | | | . . . | | | | | | |
| LC | GCI | 41-GC- | 38B4D | 38B4DAQK | | LCC | | | | | IIB | 90 | F |
| | | LCC-SIIB | | | | | | | | | | | |
| LC | Bioch | 90-(39)- | A504114 | | | LCC | | | UN | UN | | | F |
| | | Bc-LCC | | | | | | | | | | | |
| LC | Bioch | 91-(87)- | A609165 | | | LCC | | | 3 | UN | | | F |
| | | Bc-LCC | | | | | | | | | | | |
| LN | Aster | 56-As-N- | 13094 | 19763 | 19763A1 | Norm-L | PM | | | | | | M |
| | | PM | | | | | | | | | | | |
| LN | Aster | 57-As-N- | 19174 | 40654 | 40654A2 | Norm-L | PM | | | | | | F |
| | | PM | | | | | | | | | | | |
| LN | Aster | 58-As-N- | 13128 | 19642 | 19642A1 | Norm-L | PM | | | | | | F |
| | | PM | | | | | | | | | | | |
| LN | Aster | 59-As-N- | 14374 | 20548 | 20548C1 | Norm-L | PM | (Right), | | | | | F |
| | | PM | | | | | | Lobe | | | | | |
| | | | | | | | | Supe- | | | | | |
| | | | | | | | | rior | | | | | |
| LN | Amb | 60-(99)- | 36856 | | | N-PM | PM | | | | | | M |
| | | Am-N | | | | | | | | | | | |
| | | PM | | | | | | | | | | | |
| LN | Amb | 61-(96)- | 36853 | | | N-PM | PM | | | | | | F |
| | | Am-N | | | | | | | | | | | |
| | | PM | | | | | | | | | | | |
| LN | Amb | 62-(97)- | 36854 | | | N-PM | PM | | | | | | M |
| | | Am-N | | | | | | | | | | | |
| | | PM | | | | | | | | | | | |
| LN | Amb | 63-(93)- | 111P0103A | | | N-PM | PM- | | | | | | F |
| | | Am-N | | | | | ICH | | | | | | |
| | | PM | | | | | | | | | | | |
| LN | Amb | 64-(98)- | 36855 | | | N-PM | PM | | | | | | F |
| | | Am-N | | | | | | | | | | | |
| | | PM | | | | | | | | | | | |
| LN | Bioch | 67-(50)- | A503385 | | | N-PM | PM | | | | | | M |
| | | Bc-N PM | | | | | | | | | | | |
| LN | Bioch | 68-(92)- | A503204 | | | N-PM | PM | | | | | | M |
| | | Bc-N PM | | | | | | | | | | | |
| LN | Bioch | 69-(91)- | A607257 | | | N-P2- | PM | | | | | | P2 |
| | | Bc-N PM | | | | PM | | | | | | | |
| LN | Bioch | 70-(90)- | A608152 | | | N-P2 | PM | | | | | | P2 |
| | | Bc-N PM | | | | PM | | | | | | | |
| LN | Bioch | 71-(48)- | A503206 | | | N-PM | PM | | | | | | M |
| | | Bc-N PM | | | | | | | | | | | |

| | | | | | # of | | | | | | | | | | |
|---|---|---|---|---|---|---|---|---|---|---|---|---|---|---|---|
| | | | | | Y. | | | | | | | | | | |
| | | | | # Cig. | Use | # Y. | | | | | | | | Cause | |
| | | | Smoking | Per | of | off | Sm | Sm | Dr | # | HT | | Recovery | of | Exc. |
| Tissue | age | Ethnic B | Status | day | Tobacco | Tobacco | P Y? | ppl | Al | Dr | (CM) | BMI | Type | Death | Y. |
| LC | 63 | WCAU | Prev | 20 | 15 | 27 | N | — | Y | 0 | 165 | 25.3 | Surg | | 2001 |
| | | | U. | | | | | | | | | | | | |
| LC | 56 | WCAU | Prev | 15 | 28 | 10 | Y | 1 | Y | 6 | 165 | 23 | Surg | | 2002 |
| | | | U. | | | | | | | | | | | | |
| LC | 61 | | | | | | | | | | | | | | |
| LC | 50 | | | | | | | | | | | | | | |
| LC | 68 | WCAU | Nev | — | — | — | N | — | N | — | 175 | 27.3 | Surg | | 2001 |
| | | | U. | | | | | | | | | | | | |
| LC | 64 | WCAU | Prev | 15 | 40 | 7 | Y | 1 | N | 0 | 157 | 19.6 | Surg | | 2003 |
| | | | U. | | | | | | | | | | | | |
| LC | 58 | WCAU | Prev | 10 | 47 | 0 | Y | 2 | N | — | 170 | 24.6 | Surg | | 2004 |
| | | | U. | | | | | | | | | | | | |
| LC | 65 | WCAU | Curr | 6 | 30 | — | Y | 1 | N | — | 168 | 21 | Surg | | 2004 |
| | | | U. | | | | | | | | | | | | |
| LC | 59 | WCAU | Curr | 20 | 40 | — | N | — | N | — | 160 | 23.9 | Surg | | 2004 |
| | | | U. | | | | | | | | | | | | |
| LC | 69 | WCAU | Curr | 30 | 52 | — | Y | 4 | N | — | 157 | 34.8 | Surg | | 2005 |
| | | | U. | | | | | | | | | | | | |
| LC | 60 | WCAU | Curr | 40 | 40 | — | N | — | N | — | 163 | 31.8 | Surg | | 2002 |
| | | | U. | | | | | | | | | | | | |
| LC | 68 | WCAU | Prev | 5 | 4 | 43 | N | — | N | — | 165 | 22.3 | Surg | | 2003 |
| | | | U. | | | | | | | | | | | | |
| LC | 69 | WCAU | Curr | 10 | — | — | | — | N | — | 183 | 30.5 | Surg | | 2002 |
| | | | U. | | | | | | | | | | | | |
| LC | 62 | WCAU | Prev | 6 | 40 | 6 | N | — | Y | 0 | 160 | 27 | Surg | | 2004 |
| | | | U. | | | | | | | | | | | | |
| LC | 56 | WCAU | Curr | 30 | 25 | — | Y | 1 | N | — | 180 | 36.4 | Surg | | 2001 |
| | | | U. | | | | | | | | | | | | |
| LC | 61 | WCAU | Curr | 30 | 36 | — | Y | 1 | N | — | 163 | 25.1 | Surg | | 2004 |
| | | | U. | | | | | | | | | | | | |
| LC | 54 | | | | | | | | | | | | | | |
| LC | 62 | | | | | | | | | | | | | | |
| LC | 57 | | | | | | | | | | | | | | |
| LC | 64 | | | | | | | | | | | | | | |
| LC | 65 | | | | | | | | | | | | | | |
| LC | 74 | | | | | | | | | | | | | | |
| LC | 64 | | | | | | | | | | | | | | |
| LC | 68 | | | | | | | | | | | | | | |
| LC | 56 | | | | | | | | | | | | | | |
| LC | 68 | | | | | | | | | | | | | | |
| LC | 67 | CAU | Curr | 11-20 | 31-40 | | | | O | | 163 | 28.6 | Surg | | 2003 |
| | | | U. | | | | | | | | | | | | |
| LC | 68 | WCAU | Prev | 10 | 20 | 0 | N | — | N | — | 157 | 22.9 | Surg | | 2004 |
| | | | U. | | | | | | | | | | | | |
| LC | 62 | WCAU | Prev | 20 | 50 | 0 | Y | 5 | N | — | 175 | 25.5 | Surg | | 2005 |
| | | | U. | | | | | | | | | | | | |
| LC | 73 | CAU | Prev | | | | | | O | | 170 | 22.1 | Surg | | 2004 |
| | | | U. | | | | | | | | | | | | |
| LC | 66 | CAU | Prev | 11-20 | 45 | | | | P | | 178 | 33.8 | Surg | | 2005 |
| | | | U. | | | | | | | | | | | | |
| LC | 65 | CAU | Curr | 6-10 | 41-50 | | | | O | | 176 | 22 | Surg | | 2002 |
| | | | U. | | | | | | | | | | | | |
| LC | 44 | WCAU | Prev | 20 | 20 | 0 | Y | 2 | N | — | 155 | 22.7 | Surg | | 2004 |
| | | | U. | | | | | | | | | | | | |
| LC | 68 | WCAU | Prev | 40 | 40 | 0 | Y | 2 | N | — | 170 | 23.2 | Surg | | 2004 |
| | | | U. | | | | | | | | | | | | |
| LC | 58 | WCAU | Prev | 50 | 40 | 1 | Y | 2 | N | — | 173 | 27.4 | Surg | | 2004 |
| | | | U. | | | | | | | | | | | | |
| LC | 65 | | | | | | | | | | | | | | |
| LC | 72 | | | | | | | | | | | | | | |
| LC | 78 | | | | | | | | | | | | | | |
| LC | 62 | | | | | | | | | | | | | | |
| LC | 68 | | | | | | | | | | | | | | |
| LC | 74 | | | | | | | | | | | | | | |
| LC | 63 | | | | | | | | | | | | | | |
| LC | 58 | | | | | | | | | | | | | | |
| LC | 68 | | | | | | | | | | | | | | |
| LC | 62 | WCAU | Prev | 20 | 35 | 0.15 | Y | 2 | N | — | 165 | 19.8 | Surg | | 2003 |
| | | | U. | | | | | | | | | | | | |
| LC | 30 | | | | | | | | | | | | | | |
| LC | 34 | | | | | | | | | | | | | | |
| LC | 47 | | | | | | | | | | | | | | |
| LC | 52 | | | | | | | | | | | | | | |
| LC | 59 | | | | | | | | | | | | | | |
| LC | 59 | | | | | | | | | | | | | | |
| LC | 66 | | | | | | | | | | | | | | |
| LC | | | | | | | | | | | | | | | |
| LN | 22 | CAU | Nev | | | | | | NU | | 0 | 0 | Surg | | 2003 |
| | | | U. | | | | | | | | | | | | |
| LN | 26 | CAU | Nev | | | | | | O | | 170 | 22.1 | Aut | CA | 2003 |
| | | | U. | | | | | | | | | | | | |
| LN | 37 | CAU | Nev | | | | | | C | | 183 | 20.9 | Aut | MCE | 2002 |
| | | | U. | | | | | | | | | | | | |
| LN | 76 | CAU | Prev | | | | | | | | 165 | 26.8 | Aut | CPul A | 2002 |
| | | | U. | | | | | | | | | | | | |
| LN | 81 | CAU | Prev | 41 or | 31-40 | | | | O | | 183 | 30.5 | Aut | CA | 2003 |
| | | | U. | more | | | | | | | | | | | |
| LC | 53 | | | | | | | | | | | | | | |
| LC | 52 | | | | | | | | | | | | | | |
| LC | 57 | | | | | | | | | | | | | | |
| LC | 67 | | | | | | | | | | | | | | |
| LC | 48 | | | | | | | | | | | | | | |
| LC | 64 | | | | | | | | | | | | | | |
| LC | 64 | | | | | | | | | | | | | | |
| LC | 64 | | | | | | | | | | | | | | |
| LC | 76 | | | | | | | | | | | | | | |
| LC | 45 | WCAU | Prev | 45 | 33 | 0 | Y | 2 | Y | 28 | 178 | 31.9 | Surg | | 2004 |
| | | | U. | | | | | | | | | | | | |
| LC | 60 | WCAU | Prev | 30 | 45 | 0 | Y | 3 | N | — | 160 | 16.8 | Surg | | 2004 |
| | | | U. | | | | | | | | | | | | |
| LC | 68 | WCAU | Prev | — | 55 | — | Y | — | N | — | 173 | 22.8 | Surg | | 2001 |
| | | | U. | | | | | | | | | | | | |
| LC | 51 | WCAU | Prev | 20 | 12 | 22 | Y | 1 | N | — | 183 | 26.6 | Surg | | 2004 |
| | | | U. | | | | | | | | | | | | |
| LC | 62 | WCAU | Prev | 40 | 40 | 0 | Y | 2 | Y | 12 | 185 | 23.1 | Surg | | 2004 |
| | | | U. | | | | | | | | | | | | |
| LC | 70 | WCAU | Prev | 30 | 50 | — | Y | 2 | Y | 13 | 168 | 20.7 | Surg | | 2002 |
| | | | U. | | | | | | | | | | | | |
| LC | 35 | | | | | | | | | | | | | | |
| LC | 47 | | | | | | | | | | | | | | |
| LN | 0 | CAU | Prev | 21-40 | 41-50 | | | | P | | 175 | 25.1 | Aut | IC | |
| | | | U. | | | | | | | | | | | | |
| LN | 69 | CAU | Curr | 21-40 | 31-50 | | | | P | | 165 | 22.4 | Aut | CPul A | 2005 |
| | | | U. | | | | | | | | | | | | |
| LN | 75 | CAU | | | | | | | | | 160 | 21.5 | Aut | CPul A | 2004 |
| LN | 75 | CAU | | | | | | | | | 175 | 32.7 | Aut | Cer A | 2004 |
| LN | 31 | | | | | | | | | | | | | | |
| LN | 43 | | | | | | | | | | | | | | |
| LN | 46 | | | | | | | | | | | | | | |
| LN | 61 | | | | | | | | | | | | | | |
| LN | 72 | | | | | | | | | | | | | | |
| LN | 28 | | | | | | | | | | | | | | |
| LN | 28 | | | | | | | | | | | | | | |
| LN | 24, 29 | | | | | | | | | | | | | | |
| LN | 27, 28 | | | | | | | | | | | | | | |
| LN | 44 | | | | | | | | | | | | | | |

TABLE 1_6_1

| Key | Full Name |
|---|---|
| # Cig. Per day | Number of Cigarettes per day |
| # Dr | Number of Drinks |
| # of Y. Use of Tobacco | Number of Years Using Tobacco |
| # Y. off Tobacco | Number of Years Off Tobacco |
| AC | Alveolus carcinoma |
| Aden | ADENOCARCINOMA |
| Amb | Ambion |
| Aster | Asterand |
| Aut | Autopsy |
| BC | BRONCHIOLOALVEOLAR CARCINOMA |
| Bioch | Biochain |
| C | Current Use |
| CA | Cardiac arrest |
| CAU | Caucasian |
| Cer A | Cerebrovascular accident |
| CPul A | Cardiopulmonary arrest |
| CS | Cancer Stage |
| Curr U. | Current Use |
| Diag | Diagnosis |
| Dr Al | Drink Alcohol? |
| Exc Y. | Excision Year |
| Gen | Gender |
| Gr | Grade |
| Height | HT |
| IC | Ischemic cardiomyopathy |
| LC | Lung Cancer |
| LCC | LARGE CELL CARCINOMA |
| LCNC | Large Cell Neuroendocrine Carcinoma |
| LN | Lung Normal |
| MCE | Massive cerebral edema |
| N | No |
| NC | NEUROENDOCRINE CARCINOMA |
| Nev. U. | Never Used |
| Norm-L | Normal Lung |
| N-P2-PM | Normal (Pool 2)- PM |
| N-PM | Normal-PM |
| NSCC . . . | NON-SMALL CELL CARCINOMA WITH |
| | SARCOMUTOUS TRANSFORMTAIO |
| NU | Never used |
| O | Occasional Use |
| P | Previous Use |
| P2 | Pool 2 |
| Prev U. | Previous Use |
| SCC | Squamous Cell Carcinoma |
| Sm P Y? | Have people at home smoked in past 15 yr |
| Sm ppl | If yes, how many? |
| SMCC | SMALL CELL CARCINOMA |
| SMOKE_GROWING_UP | Did people smoke at home while growing up |
| Surg | Surgical |
| Tum % | Tumor Percentage |
| WCAU | White Caucasian |
| Y | Yes |

TABLE 1_7
Tissue samples in Breast cancer testing panel

| | | | | | | | | |
|---|---|---|---|---|---|---|---|---|
| BC | GCI | 33-GC-IDC | YQ1WW | YQ1WWAUV | | IDC | | |
| | | SIIIB | | | | | | |
| BC | GCI | 34-GC-IDC | KIOE7 | KIOE7AI9 | | IDC | | |
| | | SIIIB | | | | | | |
| BC | Bioch | 70-(43)-Bc- | A609183 | | | IDC | 2 | |
| | | IDC | | | | | | |
| BC | Bioch | 71-(54)-Bc- | A605353 | | | IDC | 2 | |
| | | IDC | | | | | | |
| BC | ABS | 72-(55)-Bc- | A609179 | | | IDC | 2 | |
| | | IDC | | | | | | |
| BC | Bioch | 73-(47)-Bc- | A609221 | | | IDC | 2 | |
| | | IDC | | | | | | |
| BC | Bioch | 74-(48)-Bc- | A609222 | | | IDC | 2 | |
| | | IDC | | | | | | |
| BC | Bioch | 75-(53)-Bc- | A605151 | | | IDC | 2 | |
| | | IDC | | | | | | |
| BC | Bioch | 76-(61)-Bc- | A610029 | | | IDC | 2 | |
| | | IDC | | | | | | |
| BC | Bioch | 77-(46)-Bc- | A609177 | | | Carc | 2 | |
| | | Carci | | | | | | |
| BC | Ichilov | 78-(62)-Bc- | A609194 | | | IDC | 2 | |
| | | IDC | | | | | | |
| BC | Amb | 79-(32)-AB- | 7116T | | | MC | | T2N0M0 |
| | | Muc Carci | | | | | | |
| | | SIIA | | | | | | |
| BC | GCI | 80-(49)-Bc- | A609223 | | | IDC | 2 | |
| | | IDC | | | | | | |
| BC | GCI | 81-(45)-Bc- | A609181 | | | IDC | 2 | |
| | | IDC | | | | | | |
| BC | GCI | 82-(50)-Bc- | A609224 | | | IDC | 2 | |
| | | IDC | | | | | | |
| BC | Bioch | 83-(44)-Bc- | A609198 | | | IDC | 2 | |
| | | IDC | | | | | | |
| BC | Bioch | 84-(51)-Bc- | A605361 | | | IDC | 1 | |
| | | IDC | | | | | | |
| BC | Amb | 85-(31)-Ic- | CG-154 | | | IDC | | |
| | | IDC | | | | | | |
| BC | Aster | 35-As-ILC | 17090 | 30738 | 30738A1 | ILC | | T1cNXM0 |
| | | SI | | | | | | |
| BC | GCI | 36-GC-ILC | I35US | I35USA9G | | ILC | | |
| | | SIIA | | | | | | |
| BC | GCI | 37-GC-ILC | IS84Y | IS84YAAY | | ILC | | |
| | | SIIB | | | | | | |
| BC | Bioch | 38-(52)-Bc- | A605360 | | | ILC | 1 | |
| | | ILC | | | | | | |
| BB | Aster | 39-As-Ben | 11975 | 15478 | 15478B1 | FIBR | | |
| BB | GCI | 40-GC-Ben | ZT15M | ZT15MAMR | | FIBR | | |
| BB | GCI | 41-GC-Ben | NNP3Q | NNP3QA4V | | FIBR | | |
| BB | GCI | 42-GC-Ben | QK8IY | QK8IYALU | | FIBR | | |
| BN-PS | GCI | 43-GC-N | 83LO7 | 83LO7NEH | | NB-PS | | |
| | | PS | | | | | | |
| BN-PS | GCI | 45-GC-N | O6JBJ | O6JBJNT1 | | NB-PS | | |
| | | PS | | | | | | |
| BN-PS | GCI | 46-GC-N | E6UDD | E6UDDNCF | | NB-PS | | |
| | | PS | | | | | | |
| BN-PS | GCI | 47-GC-N | DHLR1 | DHLR1NIQ | | NB-PS | | |
| | | PS | | | | | | |
| BN-PS | GCI | 48-GC-N | JHQEH | JHQEHN4D | | NB-PS | | |
| | | PS | | | | | | |
| BN-PS | Amb | 49-(63)-Am- | 26486 | | | NB-PS | | |
| | | N PS | | | | | | |
| BN-PS | GCI | 50-GC-N | ONBFK | ONBFKNO2 | | NB-PS | | |
| | | PS | | | | | | |
| BN-PS | GCI | 51-GC-N | TG6J6 | TG6J6NNA | | NB-PS | | |
| | | PS | | | | | | |
| BN-PS | Aster | 52-GC-N | 14398 | 20021 | 20021D1 | NB-PS | | |
| | | PS | | | | | | |
| BN-PS | GCI | 54-GC-N | AJGXV | AJGXVNFC | | NB-PS | | |
| | | PS | | | | | | |
| BN-PS | GCI | 56-GC-N | HLCZX | HLCZXNLS | | NB-PS | | |
| | | PS | | | | | | |
| BN-PS | GCI | 58-GC-N | FGV8P | FGV8PNQ6 | | NB-PS | | |
| | | PS | | | | | | |
| BN-? | Aster | 59-As-N PS | 9264 | 9486 | 9486A1 | NB-PS | | |
| BN-PM | Bioch | 60-(57)-Bc- | A609233 | A609233 | | NB-PM | | |
| | | N PM | | | | | | |
| BN-PM | Bioch | 61-(59)-Bc- | A607155 | A607155 | | NB-PM | | |
| | | N PM | | | | | | |
| BN-PM | Bioch | 62-(60)-Bc- | A609234 | A609234 | | NB-PM | | |
| | | N PM | | | | | | |
| BN-PM | Amb | 63-(66)-Am- | 36678 | 36678 | | NB-PM | | |
| | | N PM | | | | | | |
| BN-PM | Amb | 64-(64)-Am- | 23036 | 23036 | | NB-PM | | |
| | | N PM | | | | | | |
| BN-PM | Amb | 65-(65)-Am- | 31410 | 31410 | | NB-PM | | |
| | | N PM | | | | | | |
| BN-PM | Amb | 66-(67)-Am- | 073P010602086A | 073P010602086A | | NB-PM | | |
| | | N PM | | | | | | |
| BN-PM | Bioch | 67-(58)-Bc- | A609232 | A609232 | | NB-PM | | |
| | | N PM | | | | | | |
| BN-PM | Aster | 68-As-N | 8862 | 8766 | 8766B1 | NB-PM | | |
| | | PM | | | | | | |
| BN-PM | Aster | 69-As-N | 8457 | 7928 | 7928M1 | NB-PM | | |
| | | PM | | | | | | |

| | | | | | | | | | | | | | | |
|---|---|---|---|---|---|---|---|---|---|---|---|---|---|---|
| BC | IIIB | 60 | 62 | Pre-M | 78 | 163 | 29.5 | WCAU | 0 | — | — | — | Surg | 1940 |
| BC | IIIB | 55 | 65 | Post-M | 73.5 | 157 | 29.6 | WCAU | 2 | 2 | 18 | 2 | Surg | 1939 |
| BC | | | 40 | | | | | | | | | | | |
| BC | | | 41 | | | | | | | | | | | |
| BC | | | 42 | | | | | | | | | | | |
| BC | | | 42 | | | | | | | | | | | |
| BC | | | 44 | | | | | | | | | | | |
| BC | | | 44 | | | | | | | | | | | |
| BC | | | 46 | | | | | | | | | | | |
| BC | | | 48 | | | | | | | | | | | |
| BC | | | 51 | | | | | | | | | | | |
| BC | IIA | | 54 | | | | | | | | | | | |
| BC | | | 54 | | | | | | | | | | | |
| BC | | | 58 | | | | | | | | | | | |
| BC | | | 69 | | | | | | | | | | | |
| BC | | | 77 | | | | | | | | | | | |
| BC | | | 79 | | | | | | | | | | | |
| BC | | | 83 | | | | | | | | | | | |
| BC | I | 100 | 50 | | 94 | 170 | 32.5 | W | 2 | 2 | | | Surg | |
| BC | IIA | 60 | 70 | | 77.1 | 178 | 24.4 | WCAU | — | — | — | — | Surg | 1932 |
| BC | IIB | 65 | 67 | Post-M | 62.6 | 163 | 23.7 | WCAU | 2 | 2 | 16 | 0 | Surg | 1934 |
| BC | | | 60 | | | | | | | | | | | |
| BB | | 100 | 24 | Pre-M | 80 | 164 | 29.7 | CAU | 2 | 0 | | | Surg | |
| BB | | 100 | 34 | | 57.6 | 165 | 21.1 | WCAU | | | | | Surg | 1967 |
| BB | | 95 | 54 | | 56.7 | 157 | 22.9 | WCAU | | | | | Surg | 1948 |
| BB | | 100 | 41 | | 59 | 173 | 19.7 | WCAU | | | | | Surg | 1960 |
| BN-PS | | | 32 | | 78.5 | 155 | 32.7 | WCAU | | | | | Surg | 1969 |
| BN-PS | | | 38 | | 67.1 | 173 | 22.5 | WCAU | | | | | Surg | 1963 |
| BN-PS | | | 40 | | 99.8 | 170 | 34.5 | WCAU | | | | | Surg | 1961 |
| BN-PS | | | 40 | | 90.7 | 168 | 32.3 | WCAU | | | | | Surg | 1961 |
| BN-PS | | | 41 | | 65.3 | 157 | 26.3 | WCAU | | | | | Surg | 1960 |
| BN-PS | | | 43 | | | | | | | | | | | |
| BN-PS | | | 45 | | 81.6 | 165 | 30.0 | WCAU | | | | | Surg | 1956 |
| BN-PS | | | 46 | | 90.7 | 173 | 30.4 | WCAU | | | | | Surg | 1955 |
| BN-PS | | | 49 | Pre-M | 68 | 165 | 25.0 | CAU | 3 | 3 | | | Surg | |
| BN-PS | | | 52 | | 70 | 168 | 24.8 | WCAU | | | | | Surg | 1949 |
| BN-PS | | | 54 | | 67 | 163 | 25.2 | WCAU | | | | | Surg | 1947 |
| BN-PS | | | 61 | | 106.6 | 168 | 37.9 | WCAU | | | | | Surg | 1940 |
| BN-? | | | 0 | | 0 | 0 | 0.0 | | | | | | | |
| BN-PM | | | 34 | | | | | | | | | | Aut | |
| BN-PM | | | 35 | | | | | | | | | | Aut | |
| BN-PM | | | 36 | | | | | | | | | | Aut | |
| BN-PM | | | 45 | | | | | | | | | | Aut | |
| BN-PM | | | 57 | | | | | | | | | | Aut | |
| BN-PM | | | 63 | | | | | | | | | | Aut | |
| BN-PM | | | 64 | | | | | | | | | | Aut | |
| BN-PM | | | 65 | | | | | | | | | | Aut | |
| BN-PM | | | 74 | | 64 | 157 | 26.0 | CAU | | | | | Aut | |
| BN-PM | | | 87 | | 59 | 165 | 21.7 | CAU | | | | | Aut | |

TABLE 1_7_1

| Key | Full Name |
|---|---|
| # Live Bir | # Live Births |
| # of Preg | Number of Pregnancies |
| Amb | Ambion |
| Aster | Asterand |
| Aut | Autopsy |
| BB | Breast Benign |
| BC | Breast Cancer |
| Bioch | Biochain |
| BN | Breast Normal |
| BN-PM | Breast_Normal-PM |
| BN-PS | Breast_Normal-PS |
| Breastfeed child | Br Child |
| C Stage | Cancer Stage |
| Carc | Carcinoma |
| CAU | Caucasian |
| DCIS | Ductal Carcinoma In Situ |
| Ethnic B | Ethnic Background |
| FIBR | FIBROADENOMA |
| IDC | INFILTRATING DUCTAL CARCINOMA |
| ILC | INFILTRATING LOBULAR CARCINOMA |
| M | Menopausal |
| MC | Mucinous carcinoma |
| MS | Menopausal Status |
| NB-PM | NORMAL BREAST-PM |
| NB-PS | NORMAL BREAST-PS |
| Post-M | Post-Menopausal |
| Pre-M | Pre-Menopausal |
| Samp DIAG | Sample Diagnosis |
| Surg | Surgical |
| Tum % | Percentage of Tumor |
| W | White |
| WCAU | White Caucasian |

TABLE 1_8

| | | | sample_id | | | | | | | | |
|---|---|---|---|---|---|---|---|---|---|---|---|
| | | | (GCI)/ | | | | | | | | |
| | | | case id | TISSUE ID | | | | | | | |
| | | | (Asterand)/ | (GCI)/ | | | | | | | |
| | | | lot no. | specimen | Sample | | | | | | |
| | Source/ | sample | (old | ID | ID | | Diag | Specimen | | | |
| Tissue | Delivery | name | samples) | (Asterand) | (Asterand) | Diag | remarks | location | Gr | TNM | CS |
| CC | Asterand | 1-As- | 18036 | 31312 | 31312B1 | Aden | | Cec | 3 | TXN0M0 | 0 |
| | | AdenS0 | | | | | | | | | |
| CC | GCI | 2-GC- | 4QDH8 | 4QDH8ADT | | Aden | | Dis C | | | I |
| | | AdenoSI | | | | | | | | | |
| CC | Ichilov | 3-(7)-Ic- | CG-235 | | | AI | | Rectum | UN | | I |
| | | AdenoSI | | | | | | | | | |
| CC | GCI | 4-GC- | NTAI8 | NTAI8AOU | | Aden | | Cec | | | I |
| | | AdenoSI | | | | | | | | | |
| CC | GCI | 5-GC- | ARA7P | ARA7PAQA | | Aden | | Ret, | | | I |
| | | AdenoSI | | | | | | Low | | | |
| | | | | | | | | Ant | | | |
| CC | Ichilov | 6-(20)-Ic- | CG-249 | | | UA | | | 3 | | IIA |
| | | AdenoSIIA | | | | | | | | | |
| CC | GCI | 7-GC- | AFTS6 | AFTS6AP6 | | Aden | | | | | IIA |
| | | AdenoSIIA | | | | | | | | | |
| CC | GCI | 8-GC- | 5CYDK | 5CYDKACS | | Aden | | | | | IIA |
| | | AdenoSIIA | | | | | | | | | |
| CC | GCI | 9-GC- | XKSLS | XKSLSAF7 | | Aden | | | | | IIA |
| | | AdenoSIIA | | | | | | | | | |
| CC | GCI | 10-GC- | B4RU8 | B4RU8A8Q | | Aden | | | | | IIA |
| | | AdenoSIIA | | | | | | | | | |
| CC | GCI | 11-GC- | HB8EY | HB8EYA8I | | Aden | | | | | IIA |
| | | AdenoSIIA | | | | | | | | | |
| CC | Ichilov | 12-(22)- | CG-229C | | | Aden | | | 2 | | II |
| | | AdenoSII | | | | | | | | | |
| CC | GCI | 13-GC- | X8C7X | X8C7XATL | | Aden | | | | | IIA |
| | | AdenoSIIA | | | | | | | | | |
| CC | GCI | 14-GC- | HCP6K | HCP6KA8Z | | Aden | | | | | IIA |
| | | AdenoSIIA | | | | | | | | | |
| CC | GCI | 15-GC- | ZX4X7 | ZX4X7AXA | | Aden | | | | | IIA |
| | | AdenoSIIA | | | | | | | | | |
| CC | Asterand | 16-As- | 17915 | 31176 | 31176A1 | Aden | | | 2-3 | T3N0M0 | IIA |
| | | AdenoSIIA | | | | | | | | | |
| CC | Ichilov | 17-(1)-Ic- | CG-335 | | | Aden | | Cec | 2 | | IIA |
| | | AdenoSIIA | | | | | | | | | |
| CC | Asterand | 19-As- | 12772 | 18885 | 18885A1 | Aden | | rectum | 2 | T3NXM0 | IIA |
| | | AdenoSIIA | | | | | | | | | |
| CC | GCI | 20-GC- | JFYXP | JFYXPAMP | | Aden | | | | | IIA |
| | | AdenoSIIA | | | | | | | | | |
| CC | GCI | 21-GC- | OJXW9 | OJXW9ASR | | Aden | | | | | IIA |
| | | AdenoSIIA | | | | | | | | | |
| CC | Ichilov | 22-(28)- | CG-284 | | | Aden | | sigma | 2 | | IIA |
| | | Ic- | | | | | | | | | |
| | | AdenoSIIA | | | | | | | | | |
| CC | Ichilov | 23-(10)- | CG-311 | | | Aden | | Sig Col | 1-2 | | e IIA |
| | | Ic- | | | | | | | | | |
| | | AdenoSIIA | | | | | | | | | |
| CC | Ichilov | 24-(14)- | CG-222 (2) | | | WP | | Rectum | | | III |
| | | Ic- | | | | Aden | | | | | |
| | | AdenoSIII | | | | | | | | | |
| CC | Ichilov | 25-(23)- | CG-282 | | | MA | | sigma | UN | | III |
| | | Ic- | | | | | | | | | |
| | | AdenoSIII | | | | | | | | | |
| CC | GCI | 26-GC- | OTPI7 | OTPI7AWY | | Aden | | | | | III |
| | | AdenoSIII | | | | | | | | | |
| CC | GCI | 27-GC- | IG9NK | IG9NKAD3 | | MA | | | | | III |
| | | AdenoSIII | | | | | | | | | |
| CC | GCI | 28-GC- | 53OM7 | 53OM7AGL | | Aden | | | | | III |
| | | AdenoSIII | | | | | | | | | |
| CC | GCI | 29-GC- | BLUW6 | BLUW6A6Y | | Aden | | | | | III |
| | | AdenoSIII | | | | | | | | | |
| CC | GCI | 30-GC- | VZ6QA | VZ6QAAFA | | Aden | | RECTUM | | | III |
| | | AdenoSIII | | | | | | | | | |
| CC | Ichilov | 31-(6)-Ic- | CG-303 (3) | | | Aden | | | 1-2 | | III |
| | | AdenoSIII | | | | | | | | | |
| CC | Ichilov | 32-(2)-Ic- | CG-307 | | | Aden | | Cecum | 2 | | III |
| | | AdenoSIII | | | | | | | | | |
| CC | Ichilov | 33-(11)- | CG-337 | | | Aden | | | 1-2 | | III |
| | | Ic- | | | | | | | | | |
| | | AdenoSIII | | | | | | | | | |
| CC | Asterand | 34-As- | 18462 | 40971 | 40971A1 | TA | | Sig Col | 2 | TXN2M0 | IIIC |
| | | AdenoSIIIC | | | | | | | | | |
| CC | Ichilov | 35-(13)- | CG-290 | | | Aden | | Rect | 2 | | IV |
| | | Ic- | | | | | | Col | | | |
| | | AdenoSIV | | | | | | | | | |
| CC | GCI | 36-GC- | 7D7QV | 7D7QVAE6 | | Aden | | | | | IV |
| | | AdenoSIV | | | | | | | | | |
| CC | GCI | 37-GC- | 38U4V | 38U4VAA4 | | Aden | | | | | IV |
| | | AdenoSIV | | | | | | | | | |
| CC | Ichilov | 38-(9)-Ic- | CG-297 | | | Aden | | Rectum | 2 | | IV |
| | | AdenoSIV | | | | | | | | | |
| CC | Ichilov | 71-(16)- | CG-278C | | | Aden | | | 2 | | |
| | | Ic-Adeno | | | | | | | | | |
| CC | Ichilov | 72-(4)-Ic- | CG-276 | | | Carc | | | 3 | | |
| | | Adeno | | | | | | | | | |
| CC | Ichilov | 73-(17)- | CG-163 | | | Aden | | Rectum | 2 | | |
| | | Ic-Adeno | | | | | | | | | |
| CC | Ichilov | 74-(5)-Ic- | CG-308 | | | Aden | | Col Sig | 2 | | |
| | | Adeno | | | | | | | | | |
| CC | Ichilov | 75-(72)- | CG-309 | | | Aden | | | 3 | | |
| | | Ic-Adeno | | | | | | | | | |
| CC | Ichilov | 76-(18)- | CG-22C | | | Aden | | | UN | | |
| | | Ic-Adeno | | | | | | | | | |
| CC | Ichilov | 78-(21)- | CG-18C | | | Aden | | | UN | | |
| | | Ic-Adeno | | | | | | | | | |
| CC | Ichilov | 79-(24)- | CG-12 | | | Aden | | | UN | | |
| | | Ic-Adeno | | | | | | | | | |
| CC | Ichilov | 80-(25)- | CG-2 | | | Aden | | | UN | | |
| | | Ic-Adeno | | | | | | | | | |
| CC | biochain | 82-(61)- | A606258 | | | Aden, | | | 2 | | |
| | | Bc- | | | | Ulcer | | | | | |
| | | Adeno | | | | | | | | | |
| CC | biochain | 83-(57)- | A609150 | | | Aden | | | 3 | | |
| | | Bc- | | | | | | | | | |
| | | Adeno | | | | | | | | | |
| CC | biochain | 84-(56)- | A609148 | | | Aden | | | 2 | | |
| | | Bc- | | | | | | | | | |
| | | Adeno | | | | | | | | | |
| CN | GCI | 50-GC-N | XVPZ2 | XVPZ2NDD | | NC | Div | | | | |
| | | PS | | | | | | | | | |
| CN | GCI | 51-GC-N | CDSUV | CDSUVNR3 | | NC | CU | | | | |
| | | PS | | | | | | | | | |
| CN | GCI | 52-GC-N | GP5KH | GP5KHAOC | | NC | Div | | | | |
| | | PS | | | | | | | | | |
| CN | GCI | 53-GC-N | YUZNR | YUZNRNDN | | NC | Divs | Sig Col | | | |
| | | PS | | | | | | | | | |
| CN | GCI | 54-GC-N | 28QN6 | 28QN6NI1 | | NC | TS Aden | RT Col | | | |
| | | PS | | | | | | | | | |
| CN | GCI | 55-GC-N | GV6N8 | GV6N8NG9 | | NC | Divs, | | | | |
| | | PS | | | | | PA | | | | |
| CN | GCI | 56-GC-N | ZJ17R | ZJ17RNIH | | NC | Tub | RT Col | | | |
| | | PS | | | | | Aden | | | | |
| CN | GCI | 57-GC-N | 2EEBJ | 2EEBJN2Q | | NC | Div/Chr | | | | |
| | | PS | | | | | Infl | | | | |
| CN | GCI | 58-GC-N | 68IX5 | 68IX5N1H | | NC | Chr Div | LT Col | | | |
| | | PS | | | | | | | | | |
| CN | GCI | 59-GC-N | 9GEGL | 9GEGLN1V | | NC | Ext | Sig Col | | | |
| | | PS | | | | | Divs | | | | |
| CN | GCI | 60-GC-N | PKU8O | PKU8OAJ3 | | NC | Divs, | Sig Col | | | |
| | | PS | | | | | Chr | | | | |
| | | | | | | | Div . . . | | | | |
| CN | Asterand | 61-As-N | 22903 | 74452 | 74452B1 | NC | MU | | | | |
| | | PS | | | | | w/MI | | | | |
| CN | Asterand | 62-As-N | 16364 | 31802 | 31802B1 | NC | UC | | | | |
| | | PS | | | | | | | | | |
| CN | biochain | 63-(65)- | A607115 | | | N-PM | PM | | | | |
| | | Bc-N PM | | | | | | | | | |
| CN | Ambion | 64-(71)- | 071P10B | | | N-PM | PM | | | | |
| | | Am-N | | | | | | | | | |
| | | PM | | | | | | | | | |
| CN | biochain | 65-(66)- | A609262 | | | N-PM | PM | | | | |
| | | Bc-N PM | | | | | | | | | |
| CN | biochain | 66-(63)- | A609260 | | | N-PM | PM | | | | |
| | | Bc-N PM | | | | | | | | | |
| CN | biochain | 67-(62)- | A608273 | | | N-PM | PM | | | | |
| | | Bc-N PM | | | | | | | | | |
| CN | biochain | 68-(64)- | A609261 | | | N-PM | PM | | | | |
| | | Bc-N PM | | | | | | | | | |
| CN | biochain | 69-(41)- | A501156 | | | N-PM | PM | | | | |
| | | Bc-N PM | | | | | | | | | |
| CN | biochain | 70-(67)- | A406029 + | | | N-PM | PM | | | | |
| | | Bc-N PM | A411078 | | | P10 | | | | | |

| | | | | | | | | | | Dr. | | | |
|---|---|---|---|---|---|---|---|---|---|---|---|---|---|
| | | | | | | HT | WT | | Alcohol | per | Alc. | Recovery | Exc. |
| Tissue | CS2 | Tumor % | Gender | age | Ethnic B | (CM) | (KG) | BMI | Status | day | Dur. | Type | Y. |
| CC | | 80 | F | 43 | CAU | 169 | 65 | 22.8 | NU | | | Auto | 2004 |
| CC | Duke A | 85 | F | 44 | WCAU | 170 | 61.2 | 21.1 | Y | 4 | | Surg | |
| CC | Duke A | | F | 66 | | | | | | | | | |
| CC | Duke | 80 | M | 53 | WCAU | 180 | 110 | 33.9 | Y | — | | Surg | |
| | B1 | | | | | | | | | | | | |
| CC | Duke | 70 | F | 70 | WCAU | 165 | 66 | 24.2 | Y | 0 | | Surg | |
| | B1 | | | | | | | | | | | | |
| CC | Duke | | M | 36 | | | | | | | | | |
| | B2 | | | | | | | | | | | | |
| CC | Duke | 75 | M | 39 | WCAU | 191 | 77.1 | 21.2 | N | 0 | | Surg | |
| | B2 | | | | | | | | | | | | |
| CC | Duke | 65 | M | 44 | WCAU | 183 | 120 | 35.8 | N | — | | Surg | |
| | B2 | | | | | | | | | | | | |
| CC | Duke | 65 | M | 48 | WCAU | 188 | 83 | 23.5 | Y | 10 | | Surg | |
| | B2 | | | | | | | | | | | | |
| CC | Duke | 65 | F | 50 | WCAU | 165 | 79.8 | 29.3 | N | — | | Surg | |
| | B2 | | | | | | | | | | | | |
| CC | Duke | 65 | M | 53 | WCAU | 191 | 147 | 40.5 | N | — | | Surg | |
| | B2 | | | | | | | | | | | | |
| CC | Duke B | | F | 55 | | | | | | | | | |
| CC | Duke | 90 | M | 56 | WCAU | 178 | 116 | 36.6 | N | — | | Surg | |
| | B2 | | | | | | | | | | | | |
| CC | Duke | 80 | M | 58 | WCAU | 180 | 89.4 | 27.5 | Y | 4 | | Surg | |
| | B2. | | | | | | | | | | | | |
| CC | Duke | 90 | M | 60 | WCAU | 178 | 63.5 | 20.1 | Y | 5 | | Surg | |
| | B2 | | | | | | | | | | | | |
| CC | Duke | 60 | F | 64 | CAU | 171 | 82 | 28.0 | occ | 1 | 21-30 | Auto | 2004 |
| | B2 | | | | | | | | | drink/ | years | | |
| | | | | | | | | | | week | | | |
| CC | Duke | | F | 66 | | | | | | | | | |
| | B2 | | | | | | | | | | | | |
| CC | Duke | 60 | F | 67 | CAU | 164 | 76 | 28.3 | NU | | | Surg | 2004 |
| | B2 | | | | | | | | | | | | |
| CC | Duke | 60 | F | 68 | WCAU | 165 | 58.5 | 21.5 | Y | — | | Surg | |
| | B2 | | | | | | | | | | | | |
| CC | Duke | 90 | F | 69 | WCAU | 165 | 113 | 41.5 | N | — | | Surg | |
| | B2 | | | | | | | | | | | | |
| CC | Duke | | F | 72 | | | | | | | | | |
| | B2 | | | | | | | | | | | | |
| CC | Duke | | M | 88 | | | | | | | | | |
| | B2 | | | | | | | | | | | | |
| CC | Duke C | | F | 49 | | | | | | | | | |
| CC | Duke C | | M | 51 | | | | | | | | | |
| CC | Duke | 70 | F | 54 | WCAU | 155 | 58 | 24.1 | N | — | | Surg | |
| | C2 | | | | | | | | | | | | |
| CC | Duke | 90 | F | 54 | WCAU | 163 | 81.6 | 30.9 | N | — | | Surg | |
| | C2 | | | | | | | | | | | | |
| CC | Duke | 75 | F | 61 | WCAU | 183 | 107 | 31.9 | N | — | | Surg | |
| | C2 | | | | | | | | | | | | |
| CC | Duke | 85 | F | 64 | WCAU | 165 | 68.9 | 25.3 | N | — | | Surg | |
| | C2 | | | | | | | | | | | | |
| CC | Duke | 60 | M | 67 | WCAU | 180 | 71.7 | 22.0 | Y | 14 | | Surg | |
| | C2 | | | | | | | | | | | | |
| CC | Duke | | F | 77 | | | | | | | | | |
| | C2. | | | | | | | | | | | | |
| CC | Duke | | F | 89 | | | | | | | | | |
| | C2. | | | | | | | | | | | | |
| CC | Duke | | NA | NA | | | | | | | | | |
| | C2. | | | | | | | | | | | | |
| CC | | 76 | F | 68 | CAU | 165 | 82 | 30.1 | NU | | | Surg | 2005 |
| CC | Duke | | M | 47 | | | | | | | | | |
| | D. | | | | | | | | | | | | |
| CC | Duke D | 80 | F | 52 | WCAU | 163 | 63.5 | 24.0 | Y | 3 | | Surg | |
| CC | Duke D | 85 | F | 53 | WCAU | 173 | 78.9 | 26.5 | | — | | Surg | |
| CC | Duke | | M | 62 | | | | | | | | | |
| | D. | | | | | | | | | | | | |
| CC | UN | 50 | F | 60 | | | | | | | | | |
| CC | UN | 75 | M | 64 | | | | | | | | | |
| CC | UN | | M | 73 | | | | | | | | | |
| CC | UN | | F | 80 | | | | | | | | | |
| CC | UN | | F | 88 | | | | | | | | | |
| CC | UN | | NA | NA | | | | | | | | | |
| CC | UN | | NA | NA | | | | | | | | | |
| CC | UN | | NA | NA | | | | | | | | | |
| CC | UN | | NA | NA | | | | | | | | | |
| CC | UN | | M | 41 | | | | | | | | | |
| CC | UN | | F | 45 | | | | | | | | | |
| CC | UN | 40 | F | 48 | | | | | | | | | |
| CN | | 0 | F | 55 | WCAU | 168 | 87.1 | 31.0 | N | — | | Surg | |
| CN | | 0 | M | 55 | WCAU | 178 | 92.1 | 29.1 | N | — | | Surg | |
| CN | | 0 | F | 57 | WCAU | 165 | 73 | 26.8 | Y | 6 | | Surg | |
| CN | | 0 | F | 57 | WCAU | 175 | 94.8 | 31.0 | Y | 1 | | Surg | |
| CN | | 0 | M | 59 | WCAU | 185 | 105 | 30.7 | Y | 42 | | Surg | |
| CN | | 0 | F | 61 | WCAU | 160 | 61.7 | 24.1 | Y | 3 | | Surg | |
| CN | | 0 | M | 61 | WCAU | 175 | 92.1 | 30.0 | Y | — | | Surg | |
| CN | | 0 | F | 66 | WCAU | 160 | 62 | 24.2 | Y | 4 | | Surg | |
| CN | | 0 | F | 66 | WCAU | 155 | 58.1 | 24.2 | N | — | | Surg | |
| CN | | 0 | M | 68 | WCAU | 178 | 74.8 | 23.7 | N | — | | Surg | |
| CN | | 0 | F | 69 | WCAU | 157 | 77.6 | 31.3 | N | — | | Surg | |
| CN | | 0 | M | 71 | CAU | 173 | 116 | 38.8 | Occ | | | Surg | 2005 |
| CN | | 0 | F | 74 | WCAU | 157 | 63 | 25.6 | Occ | | | Surg | 2004 |
| CN | | | M | 24 | | | | | | | | | |
| CN | | | F | 34 | | | | | | | | | |
| CN | | | M | 58 | | | | | | | | | |
| CN | | | M | 61 | | | | | | | | | |
| CN | | | M | 66 | | | | | | | | | |
| CN | | | F | 68 | | | | | | | | | |
| CN | | | M | 78 | | | | | | | | | |
| CN | | | F&M | M | | | | | | | | | |
| | | | | (26-78) | | | | | | | | | |
| | | | | &F | | | | | | | | | |
| | | | | (53-77). | | | | | | | | | |

TABLE 1_8_1

| Key | Full Name |
|---|---|
| CC | Colon Cancer |
| CB | Colon Benign |
| CN | Colon Normal |
| WT | Weight |
| HT | Height |
| Aden | Adenocarcinoma |
| AI | Adenocarcinoma intramucosal |
| UA | Ulcerated adenocarcinoma |
| WP Aden | Well polypoid adeocarcinoma |
| MA | Mucinus adenocarcinoma |
| TA | Tubular adenocarcinoma |
| Carc | Carcinoma |
| TS Aden | TUBULOVILLOUS ADENOMA |
| TS Aden HGD | TUBULOVILLOUS ADENOMA with HIGH |
| | GRADE DYSPLASIA |
| NC | Normal Colon |
| N-PM | Normal PM |
| N-PM P10 | Normal PM (Pool 10) |
| Diag | Diagnosis |
| Div | DIVERTICULITIS |
| Divs w/F DIV | Diverticulosis with Focal DIVERTICULITIS |
| Chr Divs | Chronic diverticulosis |
| Divs w/DIV . . . | DIVERTICULOSIS WITH DIVERTICULITIS |
| | AND FOCAL ABSCESS FORMATION; NO |
| | MALIGNANCY |
| AD w/AF | Acute diverticulitis with abscess formation |
| CU | CECAL ULCERATION |
| Divs, PA | DIVERTICULOSIS AND PERICOLIC ABSCESS |
| Tub Aden | TUBULAR ADENOMA |
| Div/Chr Infl | DIVERTICULOSIS/CHRONIC INFLAMMATION |
| Chr Div | CHRONIC DIVERTICULITIS |
| Ext Divs | EXTENSIVE DIVERTICULOSIS |
| Divs, Chr Div . . . | DIVERTICULOSIS AND CHRONIC |
| | DIVERTICULITIS, SEROSAL FIBROSIS |
| | AND CHRONIC SEROSITIS |
| MU w/MI | Mucosal ulceration with mural inflammation |
| UC | Ulcerative colitis |
| Cec | cecum |
| Dis C | DISTAL COLON |
| Ret, Low Ant | RETROSIGMOID, LOW ANTERIOR |
| Rect Col | Rectosigmoidal colon |
| Sig col | Sigmod colon |
| Col Sig | Colon Sigma |
| RT Col | RIGHT COLON |
| Prox T Col | PROXIMAL TRANSVERSE COLON |
| LT Col | Left Colon |
| Gr | Grade |
| CS | Cancer Stage |
| Ethnic B | Ethnic background |
| NU | Never Used |
| Occ | Occasion |
| Cur U | Current use |
| Dr. per day | Drinks per day |
| Alc. Dur. | Alcohol Duration |
| Auto. | Autopsy |
| Surg. | Surgical |
| Exc. Y. | Excision Year |

TABLE 1_9
Tissue samples in normal panel:

| | Lot no. | Source | Tissue | Pathology | Sex/Age |
|---|---|---|---|---|---|
| 1-Am-Colon (C71) | 071P10B | Ambion | Colon | PM IC bleed | F/43 |
| 2-B-Colon (C69) | A411078 | Biochain | Colon | PM-Pool of 10 | M(26-78)&F(53-77) |
| 3-Cl-Colon (C70) | 1110101 | Clontech | Colon | PM-Pool of 3 sudden death | M&F(20-50) |
| 4-Am-Small Intestine | 091P0201A | Ambion | Small Intestine | PM ICH | M/85 |
| 5-B-Small Intestine | A501158 | Biochain | Small Intestine | PM | M/63 |
| 6-B-Rectum | A605138 | Biochain | Rectum | PM | M/25 |
| 7-B-Rectum | A610297 | Biochain | Rectum | PM | M/24 |
| 8-B-Rectum | A610298 | Biochain | Rectum | PM | M/27 |
| 9-Am-Stomach | 110P04A | Ambion | Stomach | PM GSW | M/16 |
| 10-B-Stomach | A501159 | Biochain | Stomach | PM | M/24 |
| 11-B-Esophagus | A603814 | Biochain | Esophagus | PM | M/26 |
| 12-B-Esophagus | A603813 | Biochain | Esophagus | PM | M/41 |
| 13-Am-Pancreas | 071P25C | Ambion | Pancreas | PM MVA | F/25 |
| 14-CG-Pancreas | CG-255-2 | Ichilov | Pancreas | PM | M/75 |
| 15-B-Lung | A409363 | Biochain | Lung | PM-Pool of 5 | M(24-28)&F62 |
| 16-Am-Lung (L93) | 111P0103A | Ambion | Lung | PM ICH | F/61 |
| 17-B-Lung (L92) | A503204 | Biochain | Lung | PM | M/28 |
| 19-B-Ovary (O48) | A504087 | Biochain | Ovary | PM | F/51 |
| 20-B-Ovary (O46) | A504086 | Biochain | Ovary | PM | F/41 |
| 75-G-Ovary | L629FRV1 | GCI | Ovary | PS DIGESTIVE HEMORRHAGE | F/47 |
| | | | | (ALCOHOLISM) | |
| 76-G-Ovary | DWHTZRQX | GCI | Ovary | PS LEIOMYOMAS | F/42 |
| 77-G-Ovary | FDPL9NJ6 | GCI | Ovary | PS VAGINAL BLEEDING | F/56 |
| 78-G-Ovary | GWXUZN5M | GCI | Ovary | PS ABNORMAL PAP SMEARS | F/53 |
| 21-Am-Cervix | 101P0101A | Ambion | Cervix | PM Surgery | F/40 |
| 23-B-Cervix | A504089 | Biochain | Cervix | PM-Pool of 5 | F(36-55) |
| 24-B-Uterus | A411074 | Biochain | Uterus | PM-Pool of 10 | F(32-53) |
| 25-B-Uterus | A409248 | Biochain | Uterus | PM | F/35 |
| 26-B-Uterus | A504090 | Biochain | Uterus | PM-Pool of 5 | F(40-53) |
| 28-Am-Bladder | 071P02C | Ambion | Bladder | PM GSW | M/28 |
| 29-B-Bladder | A504088 | Biochain | Bladder | PM-Pool of 5 | M(26-44)&F30 |
| 30-Am-Placenta | 021P33A | Ambion | Placenta | PB | F/33 |
| 31-B-Placenta | A410165 | Biochain | Placenta | PB | F/26 |
| 32-B-Placenta | A411073 | Biochain | Placenta | PB-Pool of 5 | F(24-30) |
| 33-B-Breast (B59) | A607155 | Biochain | Breast | PM | F/36 |
| 34-Am-Breast (B63) | 26486 | Ambion | Breast | PS bilateral breast reduction | F/43 |
| 35-Am-Breast (B64) | 23036 | Ambion | Breast | PM lung cancer | F/57 |
| 36-Cl-Prostate (P53) | 1070317 | Clontech | Prostate | PM-Pool of 47 sudden death | M(14-57) |
| 37-Am-Prostate | 061P04A | Ambion | Prostate | PM IC bleed | M/47 |
| (P42) | | | | | |
| 38-Am-Prostate | 25955 | Ambion | Prostate | PM head trauma | M/62 |
| (P59) | | | | | |
| 39-Am-Testis | 111P0104A | Ambion | Testis | PM GSW | M/25 |
| 40-B-Testis | A411147 | Biochain | Testis | PM | M/74 |
| 41-Cl-Testis | 1110320 | Clontech | Testis | PM-Pool of 45 sudden death | M(14-64) |
| 42-CG-Adrenal | CG-184-10 | Ichilov | Adrenal | PM | F/81 |
| 43-B-Adrenal | A610374 | Biochain | Adrenal | PM | F/83 |
| 44-B-Heart | A411077 | Biochain | Heart | PM-Pool of 5 | M(23-70) |
| 45-CG-Heart | CG-255-9 | Ichilov | Heart focal | PM | M/75 |
| | | | fibrosis | | |
| 46-CG-Heart | CG-227-1 | Ichilov | Heart | PM | F/36 |
| 47-Am-Liver | 081P0101A | Ambion | Liver | PM ICH | M/64 |
| 48-CG-Liver | CG-93-3 | Ichilov | Liver | PM | F/19 |
| 49-CG-Liver | CG-124-4 | Ichilov | Liver of fetus | PM | fetus |
| 50-Cl-BM | 1110932 | Clontech | Bone Marrow | PM-Pool of 8 sudden death | M&F(22-65) |
| 51-CGEN-Blood | WBC#5 | CGEN | Blood | — | M |
| 52-CGEN-Blood | WBC#4 | CGEN | Blood | — | M |
| 53-CGEN-Blood | WBC#3 | CGEN | Blood | — | M |
| 54-CG-Spleen | CG-267 | Ichilov | Spleen | PM | F/25 |
| 55-CG-Spleen | 111P0106B | Ambion | Spleen | PM GSW | M/25 |
| 56-CG-Spleen | A409246 | Biochain | Spleen | PM | F/12 |
| 57-CG-Thymus | CG-98-7 | Ichilov | Thymus | PM | F/28 |
| 58-Am-Thymus | 101P0101A | Ambion | Thymus | PM head injury | M/14 |
| 59-B-Thymus | A409278 | Biochain | Thymus | PM | M/28 |
| 60-B-Thyroid | A610287 | Biochain | Thyroid | PM | M/27 |
| 61-B-Thyroid | A610286 | Biochain | Thyroid | PM | M/24 |
| 62-CG-Thyroid | CG-119-2 | Ichilov | Thyroid | PM | F/66 |
| 63-Cl-Salivary Gland | 1070319 | Clontech | Salivary Gland | PM-Pool of 24 sudden death | M&F15-60 |
| 64-Am-Kidney | 111P0101B | Ambion | Kidney | PM ICH | M60 |
| 65-Cl-Kidney | 1110970 | Clontech | Kidney | PM-Pool of 14 sudden death | M&F18-59 |
| 66-B-Kidney | A411080 | Biochain | Kidney | PM-Pool of 5 | M24-46 |
| 67-CG-Cerebellum | CG-183-5 | Ichilov | Cerebellum | PM | M/74 |
| 68-CG-Cerebellum | CG-212-5 | Ichilov | Cerebellum | PM | M/54 |
| 69-B-Brain | A411322 | Biochain | Brain | PM | M/28 |
| 70-Cl-Brain | 1120022 | Clontech | Brain | PM | — |
| 71-B-Brain | A411079 | Biochain | Brain | PM-Pool of 2 | M27-28 |
| 72-CG-Brain | CG-151-1 | Ichilov | Brain | PM | F/86 |
| 73-Am-Skeletal | 101P013A | Ambion | Skeletal Muscle | PM head injury | F/28 |
| Muscle | | | | | |
| 74-Cl-Skeletal | 1061038 | Clontech | Skeletal Muscle | PM-Pool of 2 sudden death | M&F43-46 |
| Muscle | | | | | |

TABLE 1_10

| | | Sample id(GCI)/case id | Tissue id (GCI)/Specimen | Sample id (Asterand)/ |
|---|---|---|---|---|
| sample name | Source | (Asterand) Lot no. | id (Asternd) | RNA id (GCI) |
| 1-(7)-Bc-Rectum | Biochain | A610297 | | |
| 2-(8)-Bc-Rectum | Biochain | A610298 | | |
| 3-GC-Colon | GCI | CDSUV | CDSUVNR3 | |
| 4-As-Colon | Asterand | 16364 | 31802 | 31802B1 |
| 5-As-Colon | Asterand | 22900 | 74446 | 74446B1 |
| 6-GC-Small bowl | GCI | V9L7D | V9L7DN6Z | |
| 7-GC-Small bowl | GCI | M3GVT | M3GVTN5R | |
| 8-GC-Small bowl | GCI | 196S2 | 196S2AJN | |
| 9-(9)-Am-Stomach | Ambion | 110P04A | | |
| 10-(10)-Bc-Stomach | Biochain | A501159 | | |
| 11-(11)-Bc-Esoph | Biochain | A603814 | | |
| 12-(12)-Bc-Esoph | Biochain | A603813 | | |
| 13-As-Panc | Asterand | 8918 | 9442 | 9442C1 |
| 14-As-Panc | Asterand | 10082 | 11134 | 11134B1 |
| 15-(48)-Ic-Liver | Ichilov | CG-93-3 | | |
| 16-As-Liver | Asterand | 7916 | 7203 | 7203B1 |
| 17-(28)-Am-Bladder | Ambion | 071P02C | | |
| 18-(29)-Bc-Bladder | Biochain | A504088 | | |
| 19-(64)-Am-Kidney | Ambion | 111P0101B | | |
| 20-(65)-CI-Kidney | Clontech | 1110970 | | |
| 21-(66)-Bc-Kidney | Biochain | A411080 | | |
| 22-GC-Kidney | GCI | N1EVZ | N1EVZN91 | |
| 23-GC-Kidney | GCI | BMI6W | BMI6WN9F | |
| 24-(42)-Ic-Adrenal | Ichilov | CG-184-10 | | |
| 25-(43)-Bc-Adrenal | Biochain | A610374 | | |
| 26-(16)-Am-Lung | Ambion | 111P0103A | | |
| 27-(17)-Bc-Lung | Biochain | A503204 | | |
| 28-As-Lung | Asterand | 9078 | 9275 | 9275B1 |
| 29-As-Lung | Asterand | 6692 | 6161 | 6161A1 |
| 30-As-Lung | Asterand | 7900 | 7180 | 7180F1 |
| 31-(75)-GC-Ovary | GCI | L629FRV1 | | |
| 32-(76)-GC-Ovary | GCI | DWHTZRQX | | |
| 33-(77)-GC-Ovary | GCI | FDPL9NJ6 | | |
| 34-(78)-GC-Ovary | GCI | GWXUZN5M | | |
| 35-(21)-Am-Cerix | Ambion | 101P0101A | | |
| 36-GC-cervix | GCI | E2P2N | E2P2NAP4 | |
| 37-(24)-Bc-Uterus | Biochain | A411074 | | |
| 38-(26)-Bc-Uterus | Biochain | A504090 | | |
| 39-(30)-Am-Placen | Ambion | 021P33A | | |
| 40-(32)-Bc-Placen | Biochain | A411073 | | |
| 41-GC-Breast | GCI | DHLR1 | | |
| 42-GC-Breast | GCI | TG6J6 | | |
| 43-GC-Breast | GCI | E6UDD | E6UDDNCF | |
| 44-(38)-Am-Prostate | Ambion | 25955 | | |
| 45-Bc-Prostate | Biochain | A609258 | | |
| 46-As-Testis | Asterand | 13071 | 19567 | 19567B1 |
| 47-As-Testis | Asterand | 19671 | 42120 | 42120A1 |
| 48-GC-Artery | GCI | 7FUUP | 7FUUPAMP | |
| 49-GC-Artery | GCI | YGTVY | YGTVYAIN | |
| 50-Th-Blood-PBMC | Tel- | 52497 | | |
| | Hashomer | | | |
| 51-Th-Blood-PBMC | Tel- | 31055 | | |
| | Hashomer | | | |
| 52-Th-Blood-PBMC | Tel- | 31058 | | |
| | Hashomer | | | |
| 53-(54)-Ic-Spleen | Ichilov | CG-267 | | |
| 54-(55)-Ic-Spleen | Ichilov | 111P0106B | | |
| 55-(57)-Ic-Thymus | Ichilov | CG-98-7 | | |
| 56-(58)-Am-Thymus | Ambion | 101P0101A | | |
| 57-(60)-Bc-Thyroid | Biochain | A610287 | | |
| 58-(62)-Ic-Thyroid | Ichilov | CG-119-2 | | |
| 59-Gc-Sali gland | GCI | NNSMV | NNSMVNJC | |
| 60-(67)-Ic-Cerebellum | Ichilov | CG-183-5 | | |
| 61-(68)-Ic-Cerebellum | Ichilov | CG-212-5 | | |
| 62-(69)-Bc-Brain | Biochain | A411322 | | |
| 63-(71)-Bc-Brain | Biochain | A411079 | | |
| 64-(72)-Ic-Brain | Ichilov | CG-151-1 | | |
| 65-(44)-Bc-Heart | Biochain | A411077 | | |
| 66-(46)-Ic-Heart | Ichilov | CG-227-1 | | |
| 67-(45)-Ic-Heart | Ichilov | CG-255-9 | | |
| (Fibrotic) | | | | |
| 68-GC-Skel Mus | GCI | T8YZS | T8YZSN7O | |
| 69-GC-Skel Mus | GCI | Q3WKA | Q3WKANCJ | |
| 70-As-Skel Mus | Asterand | 8774 | 8235 | 8235G1 |
| 71-As-Skel Mus | Asterand | 8775 | 8244 | 8244A1 |
| 72-As-Skel Mus | Asterand | 10937 | 12648 | 12648C1 |
| 73-As-Skel Mus | Asterand | 6692 | 6166 | 6166A1 |

### Materials and Experimental Procedures

RNA preparation—RNA was obtained from ABS (Wilmington, Del. 19801, USA, absbioreagents.com), BioChain Inst. Inc. (Hayward, Calif. 94545 USA biochain.com), GOG for ovary samples—Pediatic Cooperative Human Tissue Network, Gynecologic Oncology Group Tissue Bank, Children Hospital of Columbus (Columbus Ohio 43205 USA), Clontech (Franklin Lakes, N.J. USA 07417, clontech.com), Ambion (Austin, Tex. 78744 USA, ambion.com), Asternad (Detroit, Mich. 48202-3420, USA, asterand.com), and from Genomics Collaborative Inc. a Division of Seracare (Cambridge, Mass. 02139, USA, .genomicsinc.com). Alternatively, RNA was generated from tissue samples using TRI-Reagent (Molecular Research Center), according to Manufacturer's instructions. Tissue and RNA samples were obtained from patients or from postmortem. Total RNA samples were treated with DNaseI (Ambion).

RT PCR—Purified RNA (1 μg) was mixed with 150 ng Random Hexamer primers (Invitrogen) and 500 μM dNTP in a total volume of 15.6 μl. The mixture was incubated for 5 min at 65° C. and then quickly chilled on ice. Thereafter, 5 μl of 5× SuperscriptII first strand buffer (Invitrogen), 2.41 μl 0.1M DTT and 40 units RNasin (Promega) were added, and the mixture was incubated for 10 min at 25° C., followed by further incubation at 42° C. for 2 min. Then, 1 μl (200 units) of SuperscriptII (Invitrogen) was added and the reaction (final volume of 25 μl) was incubated for 50 min at 42° C. and then inactivated at 70° C. for 15 min. The resulting cDNA was diluted 1:20 in TE buffer (10 mM Tris pH=8, 1 mM EDTA pH=8).

Real-Time RT-PCR analysis—cDNA (511), prepared as described above, was used as a template in Real-Time PCR reactions using the SYBR Green I assay (PE Applied Biosystem) with specific primers and UNG Enzyme (Eurogentech or ABI or Roche). The amplification was effected as follows: 50° C. for 2 min, 95° C. for 10 min, and then 40 cycles of 95° C. for 15 sec, followed by 60° C. for 1 min. Detection was performed by using the PE Applied Biosystem SDS 7000. The cycle in which the reactions achieved a threshold level (Ct) of fluorescence was registered and was used to calculate the relative transcript quantity in the RT reactions. Non-detected samples were assigned Ct value of 41 and were calculated accordingly. The relative quantity was calculated using the equation Q=efficiencŷ^{-Ct}. The efficiency of the PCR reaction was calculated from a standard curve, created by using serial dilutions of several reverse transcription (RT) reactions prepared from RNA purified from 5 cell lines (HCT116, H1299, DU145, MCF7, ES-2). To minimize inherent differences in the RT reaction, the resulting relative quantities were normalized to normalization factor calculated in one of the following methods as indicated in the text:

Method 1—the geometric mean of the relative quantities of the selected housekeeping (HSKP) genes was used as normalization factor.

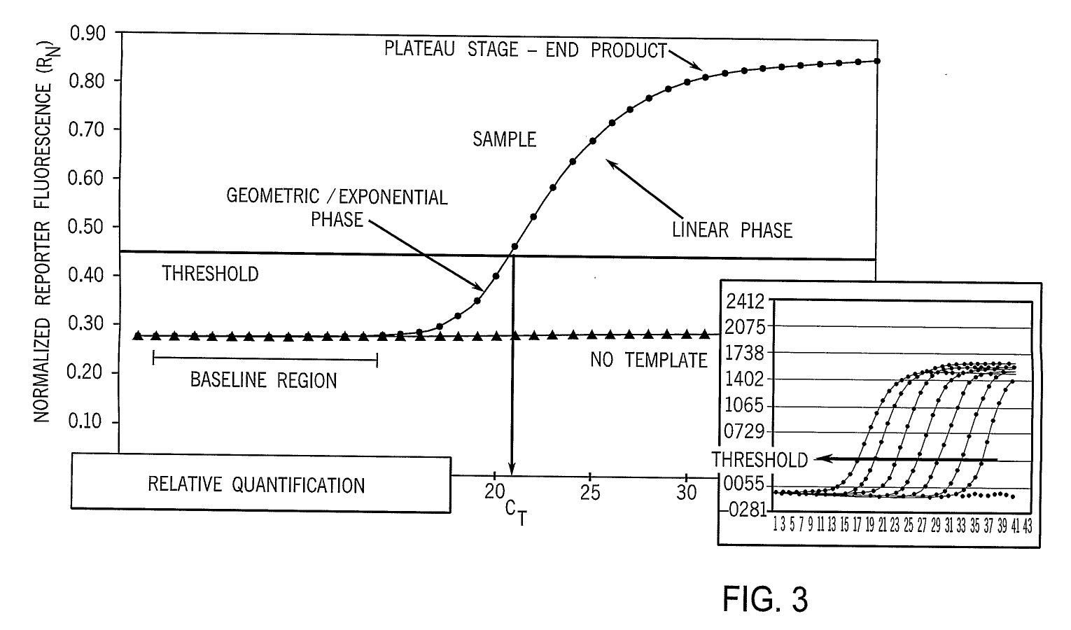

Method 2—The expression of several housekeeping (HSKP) genes was checked on every panel. The relative quantity (Q) of each housekeeping gene in each sample, calculated as described above, was divided by the median quantity of this gene in all panel samples to obtain the “relative Q rel to MED”. Then, for each sample the median of the “relative Q rel to MED” of the selected housekeeping genes was calculated and served as normalization factor of this sample for further calculations. Schematic summary of quantitative real-time PCR analysis is presented in FIG. 3. As shown, the x-axis shows the cycle number. The C_{T}=Threshold Cycle point, which is the cycle that the amplification curve crosses the fluorescence threshold that was set in the experiment. This point is a calculated cycle number in which PCR products signal is above the background level (passive dye ROX) and still in the Geometric/Exponential phase (as shown, once the level of fluorescence crosses the measurement threshold, it has a geometrically increasing phase, during which measurements are most accurate, followed by a linear phase and a plateau phase; for quantitative measurements, the latter two phases do not provide accurate measurements). The y-axis shows the normalized reporter fluorescence. It should be noted that this type of analysis provides relative quantification.

The sequences of the housekeeping genes measured in all the examples on ovarian cancer panel were as follows:

SDHA (GenBank Accession No. NM_004168
(SEQ ID NO: 335);
SDHA Forward primer (SEQ ID NO: 297):
TGGGAACAAGAGGGCATCTG
SDHA Reverse primer (SEQ ID NO: 298):
CCACCACTGCATCAAATTCATG
SDHA-amplicon (SEQ ID NO: 299):
TGGGAACAAGAGGGCATCTGCTAAAGTTTCAGATTCCATTTCTGCTCAGT
ATCCAGTAGTGGATCATGAATTTGATGCAGTGGTGG
PBGD (GenBank Accession No. BC019323
(SEQ ID NO: 336)),
PBGD Forward primer (SEQ ID NO: 300):
TGAGAGTGATTCGCGTGGG
PBGD Reverse primer (SEQ ID NO: 301):
CCAGGGTACGAGGCTTTCAAT
PBGD-amplicon (SEQ ID NO: 302):
TGAGAGTGATTCGCGTGGGTACCCGCAAGAGCCAGCTTGCTCGCATACAG
ACGGACAGTGTGGTGGCAACATTGAAAGCCTCGTACCCTGG
HPRT1 (GenBank Accession No. NM_000194
(SEQ ID NO: 337)),
HPRT1 Forward primer (SEQ ID NO: 303):
TGACACTGGCAAAACAATGCA
HPRT1 Reverse primer (SEQ ID NO: 304):
GGTCCTTTTCACCAGCAAGCT
HPRT1-amplicon (SEQ ID NO: 305) (SEQ ID NO: 305):
TGACACTGGCAAAACAATGCAGACTTTGCTTTCCTTGGTCAGGCAGTATA
ATCCAAAGATGGTCAAGGTCGCAAGCTTGCTGGTGAAAAGGACC
GAPDH (GenBank Accession No. BC026907
(SEQ ID NO: 338))
GAPDH Forward primer (SEQ ID NO: 306):
TGCACCACCAACTGCTTAGC
GAPDH Reverse primer (SEQ ID NO: 307):
CCATCACGCCACAGTTTCC
GAPDH-amplicon (SEQ ID NO: 308):
TGCACCACCAACTGCTTAGCACCCCTGGCCAAGGTCATCCATGACAACTT
TGGTATCGTGGAAGGACTCATGACCACAGTCCATGCCATCACTGCCACCC
AGAAGACTGTGGATGG

The sequences of the housekeeping genes measured in all the examples on colon cancer tissue testing panel were as follows:

PBGD (GenBank Accession No. BC019323
(SEQ ID NO: 336)),
PBGD Forward primer (SEQ ID NO: 300):
TGAGAGTGATTCGCGTGGG
PBGD Reverse primer (SEQ ID NO: 301):
CCAGGGTACGAGGCTTTCAAT
PBGD-amplicon (SEQ ID NO: 302):
TGAGAGTGATTCGCGTGGGTACCCGCAAGAGCCAGCTTGCTCGCATACAG
ACGGACAGTGTGGTGGCAACATTGAAAGCCTCGTACCCTGG
HPRT1 (GenBank Accession No. NM_000194
(SEQ ID NO: 337)),
HPRT1 Forward primer (SEQ ID NO: 303):
TGACACTGGCAAAAGAATGCA
HPRT1 Reverse primer (SEQ ID NO: 304):
GGTCCTTTTCACCAGCAAGCT
HPRT1-amplicon (SEQ ID NO: 305) (SEQ ID NO: 305):
TGACACTGGCAAAACAATGCAGACTTTGCTTTCCTTGGTCAGGCAGTATA
ATCCAAAGATGGTCAAGGTCGCAAGCTTGCTGGTGAAAAGGACC
G6PD (GenBank Accession No. NM_000402
(SEQ ID NO: 339))
G6PD Forward primer (SEQ ID NO: 309):
gaggccgtcaccaagaacat
G6PD Reverse primer (SEQ ID NO: 310):
ggacagccggtcagagctc
G6PD-amplicon (SEQ ID NO: 311):
gaggccgtcaccaagaacattcacgagtcctgcatgagccagataggctg
gaaccgcatcatcgtggagaagcccttcgggagggacctgcagagctctg
accggctgtcc
RPS27A (GenBank Accession No. NM_002954
(SEQ ID NO: 340))
RPS27A Forward primer (SEQ ID NO: 312):
CTGGCAAGCAGCTGGAAGAT
RPS27A Reverse primer (SEQ ID NO: 313):
TTTCTTAGCACCACCACGAAGTC
RPS27A-amplicon (SEQ ID NO: 314):
CTGGCAAGCAGCTGGAAGATGGACGTACTTTGTCTGACTACAATATTCAA
AAGGAGTCTACTCTTCATCTTGTGTTGAGACTTCGTGGTGGTGCTAAGAA
A

The sequences of the housekeeping genes measured in all the examples in the lung panel were as follows:

Ubiquitin (GenBank Accession No. BC000449
(SEQ ID NO: 341))
Ubiquitin Forward primer (SEQ ID NO: 315):
ATTTGGGTCGCGGTTCTTG
Ubiquitin Reverse primer (SEQ ID NO: 316):
TGCCTTGACATTCTCGATGGT
Ubiquitin-amplicon (SEQ ID NO: 317)
ATTTGGGTCGCGGTTCTTGTTTGTGGATCGCTGTGATCGTCACTTGACAA
TGCAGATCTTCGTGAAGACTCTGACTGGTAAGACCATCACCCTCGAGG T
TGAGCCCAGTGACACCATCGAGAATGTCAAGGCA
SDHA (GenBank Accession No. NM_004168
(SEQ ID NO: 335))
SDHA Forward primer (SEQ ID NO: 297):
TGGGAACAAGAGGGCATCTG
SDHA Reverse primer (SEQ ID NO: 298):
CCACCACTGCATCAAATTCATG
SDHA-amplicon (SEQ ID NO: 299):
TGGGAACAAGAGGGCATCTGCTAAAGTTTCAGATTCCATTTCTGCTCAGT
ATCCAGTAGTGGATCATGAATTTGATGCAGTGGTGG
PBGD (GenBank Accession No. BC019323
(SEQ ID NO: 336)),
PBGD Forward primer (SEQ ID NO: 300):
TGAGAGTGATTCGCGTGGG
PBGD Reverse primer (SEQ ID NO: 301):
CCAGGGTACGAGGCTTTCAAT
PBGD-amplicon (SEQ ID NO: 302):
TGAGAGTGATTCGCGTGGGTACCCGCAAGAGCCAGCTTGCTCGCATACAG
ACGGACAGTGTGGTGGCAACATTGAAAGCCTCGTACCCTGG
HPRT1 (GenBank Accession No. NM_000194
(SEQ ID NO: 337)),
HPRT1 Forward primer (SEQ ID NO: 303):
TGACACTGGCAAAACAATGCA
HPRT1 Reverse primer (SEQ ID NO: 304):
GGTCCTTTTCACCAGCAAGCT
HPRT1-amplicon (SEQ ID NO: 305) (SEQ ID NO: 305):
TGACACTGGCAAAACAATGCAGACTTTGCTTTCCTTGGTCAGGCAGTATA
ATCCAAAGATGGTCAAGGTCGCAAGCTTGCTGGTGAAAAGGACC

The sequences of the housekeeping genes measured in all the examples on breast cancer panel were as follows:

G6PD (GenBank Accession No. NM_000402
(SEQ ID NO:339))
G6PD Forward primer (SEQ ID NO: 309):
gaggccgtcaccaagaacat
G6PD Reverse primer (SEQ ID NO: 310):
ggacagccggtcagagctc
G6PD-amplicon (SEQ ID NO: 311):
gaggccgtcaccaagaacattcacgagtcctgcatgagccagataggctg
gaaccgcatcatcgtggagaagcccttcgggagggacctgcagagctctg
accggctgtcc
SDHA (GenBank Accession No. NM_004168
(SEQ ID NO:335))
SDHA Forward primer (SEQ ID NO: 297):
TGGGAACAAGAGGGCATCTG
SDHA Reverse primer (SEQ ID NO: 298):
CCACCACTGCATCAAATTCATG
SDHA-amplicon (SEQ ID NO:299):
TGGGAACAAGAGGGCATCTGCTAAAGTTTCAGATTCCATTTCTGCTCAGT
ATCCAGTAGTGGATCATGAATTTGATGCAGTGGTGG
PBGD (GenBank Accession No. BC019323
(SEQ ID NO:336)),
PBGD Forward primer (SEQ ID NO: 300):
TGAGAGTGATTCGCGTGGG
PBGD Reverse primer (SEQ ID NO: 301):
CCAGGGTACGAGGCTTTCAAT
PBGD-amplicon (SEQ ID NO: 302):
TGAGAGTGATTCGCGTGGGTACCCGCAAGAGCCAGCTTGCTCGCATACAG
ACGGACAGTGTGGTGGCAACATTGAAAGCCTCGTACCCTGG
HPRT1 (GenBank Accession No. NM_000194
(SEQ ID NO:337)),
HPRT1 Forward primer (SEQ ID NO: 303):
TGACACTGGCAAAACAATGCA
HPRT1 Reverse primer (SEQ ID NO: 304):
GGTCCTTTTCACCAGCAAGCT
HPRT1-amplicon (SEQ ID NO: 305) (SEQ ID NO: 305):
TGACACTGGCAAAACAATGCAGACTTTGCTTTCCTTGGTCAGGCAGTATA
ATCCAAAGATGGTCAAGGTCGCAAGCTTGCTGGTGAAAAGGACC

The sequences of the housekeeping genes measured in all the examples on normal tissue samples panel were as follows:

RPL19 (GenBank Accession No. NM_000981
(SEQ ID NO: 342))
RPL19 Forward primer (SEQ ID NO: 318):
TGGCAAGAAGAAGGTCTGGTTAG
RPL19 Reverse primer (SEQ ID NO: 319):
TGATCAGCCCATCTTTGATGAG
RPL19-amplicon (SEQ ID NO: 320):
TGGCAAGAAGAAGGTCTGGTTAGACCCCAATGAGACCAATGAAATCGCCA
ATGCCAACTCCCGTCAGCAGATCCGGAAGCTCATCAAAGATGGGCTGATC
A
TATA box (GenBank Accession No. NM_003194
(SEQ ID NO: 343)),
TATA box Forward primer (SEQ ID NO: 321):
CGGTTTGCTGCGGTAATCAT
TATA box Reverse primer (SEQ ID NO: 322):
TTTCTTGCTGCCAGTCTGGAC
TATA box amplicon (SEQ ID NO: 323):
CGGTTTGCTGCGGTAATCATGAGGATAAGAGAGCCACGAACCACGGCACT
GATTTTCAGTTCTGGGAAAATGGTGTGCACAGGAGCCAAGAGTGAAGAAC
AGTCCAGACTGGCAGCAAGAAA
Ubiquitin (GenBank Accession No. BC000449
(SEQ ID NO: 341))
Ubiquitin Forward primer (SEQ ID NO: 315):
ATTTGGGTCGCGGTTCTTG
Ubiquitin Reverse primer (SEQ ID NO: 316):
TGCCTTGACATTCTCGATGGT
Ubiquitin-amplicon (SEQ ID NO: 317)
ATTTGGGTCGCGGTTCTTGTTTGTGGATCGCTGTGATCGTCACTTGACAA
TGCAGATCTTCGTGAAGACTCTGACTGGTAAGACCATCACCCTCGAGG T
TGAGCCCAGTGACACCATCGAGAATGTCAAGGCA
SDHA (GenBank Accession No. NM_004168
(SEQ ID NO: 335))
SDHA Forward primer (SEQ ID NO: 297):
TGGGAACAAGAGGGCATCTG
SDHA Reverse primer (SEQ ID NO: 298):
CCACCACTGCATCAAATTCATG
SDHA-amplicon (SEQ ID NO: 299):
TGGGAACAAGAGGGCATCTGCTAAAGTTTCAGATTCCATTTCTGCTCAGT
ATCCAGTAGTGGATCATGAATTTGATGCAGTGGTGG

Oligonucleotide-Based Micro-Array Experiment Protocol-Microarray fabrication

Microarrays (chips) were printed by pin deposition using the MicroGrid II MGII 600 robot from BioRobotics Limited (Cambridge, UK). 50-mer oligonucleotides target sequences were designed by Compugen Ltd (Tel-Aviv, Ill.) as described by A. Shoshan et al, “Optical technologies and informatics”, Proceedings of SPIE. Vol 4266, pp. 86-95 (2001). The designed oligonucleotides were synthesized and purified by desalting with the Sigma-Genosys system (The Woodlands, Tex., US) and all of the oligonucleotides were joined to a C6 amino-modified linker at the 5′ end, or being attached directly to CodeLink slides (Cat #25-6700-01. Amersham Bioscience, Piscataway, N.J., US). The 50-mer oligonucleotides, forming the target sequences, were first suspended in Ultra-pure DDW (Cat # 01-866-1A Kibbutz Beit-Haemek, Israel) to a concentration of 50 μM. Before printing the slides, the oligonucleotides were resuspended in 300 mM sodium phosphate (pH 8.5) to final concentration of 150 mM and printed at 35-40% relative humidity at 21° C.

Each slide contained a total of 9792 features in 32 subarrays. Of these features, 4224 features were sequences of interest according to the present invention and negative controls that were printed in duplicate. An additional 288 features (96 target sequences printed in triplicate) contained housekeeping genes from Human Evaluation Library2, Compugen Ltd, israel. Another 384 features are E. coli spikes 1-6, which are oligos to E-Coli genes which are commercially available in the Array Control product (Array control—sense oligo spots, Ambion Inc. Austin, Tex. Cat #1781, Lot #112K06).

### Post-Coupling Processing of Printed Slides

After the spotting of the oligonucleotides to the glass (CodeLink) slides, the slides were incubated for 24 hours in a sealed saturated NaCl humidification chamber (relative humidity 70-75%).

Slides were treated for blocking of the residual reactive groups by incubating them in blocking solution at 50° C. for 15 minutes (10 ml/slide of buffer containing 0.1M Tris, 50 mM ethanolamine, 0.1% SDS). The slides were then rinsed twice with Ultra-pure DDW (double distilled water). The slides were then washed with wash solution (10 ml/slide. 4×SSC, 0.1% SDS)) at 50° C. for 30 minutes on the shaker. The slides were then rinsed twice with Ultra-pure DDW, followed by drying by centrifugation for 3 minutes at 800 rpm.

Next, in order to assist in automatic operation of the hybridization protocol, the slides were treated with Ventana Discovery hybridization station barcode adhesives. The printed slides were loaded on a Bio-Optica (Milan, Italy) hematology staining device and were incubated for 10 minutes in 50 ml of 3-Aminopropyl Triethoxysilane (Sigma A3648 lot #122K589). Excess fluid was dried and slides were then incubated for three hours in 20 mm/Hg in a dark vacuum desiccator (Pelco 2251, Ted Pella, Inc. Redding Calif.).

The following protocol was then followed with the Genisphere 900-RP (random primer), with mini elute columns on the Ventana Discovery HybStation™, to perform the microarray experiments. Briefly, the protocol was performed as described with regard to the instructions and information provided with the device itself. The protocol included cDNA synthesis and labeling. cDNA concentration was measured with the TBS-380 (Turner Biosystems. Sunnyvale, Calif.) PicoFlour, which is used with the OliGreen ssDNA Quantitation reagent and kit.

Hybridization was performed with the Ventana Hybridization device, according to the provided protocols (Discovery Hybridization Station Tuscon Ariz.).

The slides were then scanned with GenePix 4000B dual laser scanner from Axon Instruments Inc, and analyzed by GenePix Pro 5.0 software.

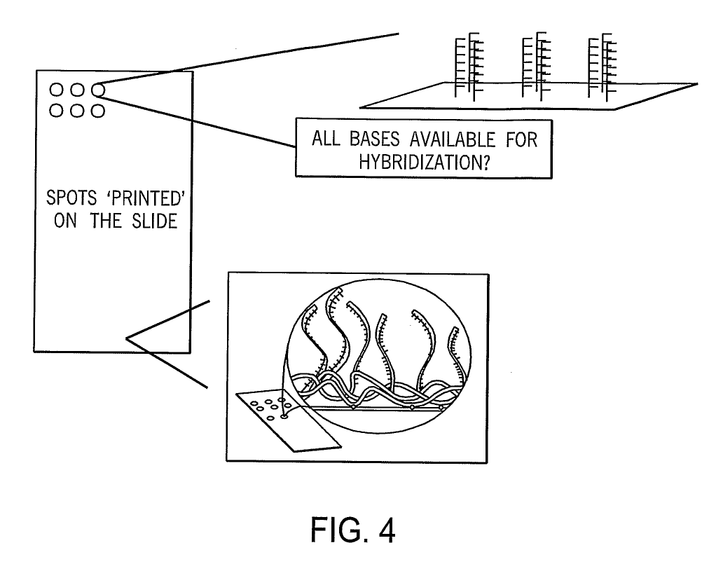

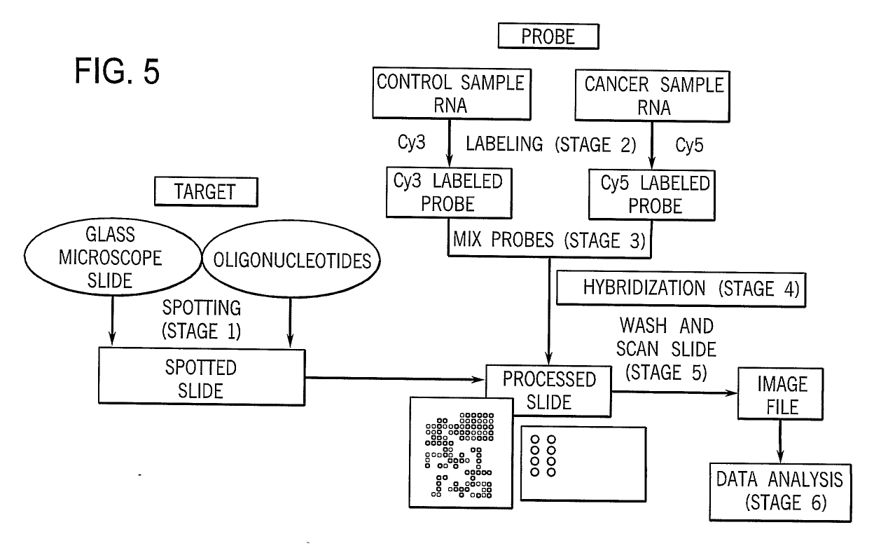

Schematic summary of the oligonucleotide based microarray fabrication and the experimental flow is presented in FIGS. 4 and 5.

Briefly, as shown in FIG. 4, DNA oligonucleotides at 25 uM were deposited (printed) onto Amersham ‘CodeLink’ glass slides generating a well defined ‘spot’. These slides are covered with a long-chain, hydrophilic polymer chemistry that creates an active 3-D surface that covalently binds the DNA oligonucleotides 5′-end via the C6-amine modification. This binding ensures that the full length of the DNA oligonucleotides is available for hybridization to the cDNA and also allows lower background, high sensitivity and reproducibility.

FIG. 5 shows a schematic method for performing the microarray experiments. It should be noted that stages on the left-hand or right-hand side may optionally be performed in any order, including in parallel, until stage 4 (hybridization). Briefly, on the left-hand side, the target oligonucleotides are being spotted on a glass microscope slide (although optionally other materials could be used) to form a spotted slide (stage 1). On the right hand side, control sample RNA and cancer sample RNA are Cy3 and Cy5 labeled, respectively (stage 2), to form labeled probes. It should be noted that the control and cancer samples come from corresponding tissues (for example, normal prostate tissue and cancerous prostate tissue). Furthermore, the tissue from which the RNA was taken is indicated below in the specific examples of data for particular clusters, with regard to overexpression of an oligonucleotide from a “chip” (microarray), as for example “prostate” for chips in which prostate cancerous tissue and normal tissue were tested as described above. In stage 3, the probes are mixed. In stage 4, hybridization is performed to form a processed slide. In stage 5, the slide is washed and scanned to form an image file, followed by data analysis in stage 6.

### ACTUAL MARKER EXAMPLES

The following examples relate to specific actual marker examples. The numbering of the tables restarts within each section.

### Description for Cluster AA383349

Cluster AA383349 features 4 transcript(s) and 25 segment(s) of interest, the names for which are given in Tables 2 and 3, respectively, the sequences themselves are given. The selected protein variants are given in table 4.

TABLE 2
Transcripts of interest
Transcript Name

AA383349_T6 (SEQ ID NO: 334)
AA383349_T7 (SEQ ID NO: 1)
AA383349_T16 (SEQ ID NO: 2)
AA383349_T17 (SEQ ID NO: 3)

TABLE 3
Segments of interest
Segment Name

AA383349_N0 (SEQ ID NO: 4)
AA383349_N1 (SEQ ID NO: 5)
AA383349_N3 (SEQ ID NO: 6)
AA383349_N5 (SEQ ID NO: 7)
AA383349_N6 (SEQ ID NO: 8)
AA383349_N32 (SEQ ID NO: 9)
AA383349_N34 (SEQ ID NO: 10)
AA383349_N2 (SEQ ID NO: 11)
AA383349_N4 (SEQ ID NO: 12)
AA383349_N9 (SEQ ID NO: 13)
AA383349_N11 (SEQ ID NO: 14)
AA383349_N12 (SEQ ID NO: 15)
AA383349_N13 (SEQ ID NO: 16)
AA383349_N14 (SEQ ID NO: 17)
AA383349_N17 (SEQ ID NO: 18)
AA383349_N18 (SEQ ID NO: 19)
AA383349_N20 (SEQ ID NO: 20)
AA383349_N23 (SEQ ID NO: 21)
AA383349_N25 (SEQ ID NO: 22)
AA383349_N27 (SEQ ID NO: 23)
AA383349_N29 (SEQ ID NO: 24)
AA383349_N30 (SEQ ID NO: 25)
AA383349_N31 (SEQ ID NO: 26)
AA383349_N33 (SEQ ID NO: 27)
AA383349_N35 (SEQ ID NO: 28)

TABLE 4
Proteins of interest

| Protein Name | Corresponding Transcript(s) |
|---|---|
| AA383349_P4 (SEQ ID NO: 33) | AA383349_T6 (SEQ ID NO: 334) |
| AA383349_P5 (SEQ ID NO: 34) | AA383349_T7 (SEQ ID NO: 1) |
| AA383349_P11 (SEQ ID NO: 35) | AA383349_T16 (SEQ ID NO: 2) |
| AA383349_P12 (SEQ ID NO: 36) | AA383349_T17 (SEQ ID NO: 3) |

These sequences are variants of the known protein Hypothetical protein (SEQ ID NO: 29) (SwissProt accession identifier Q6DKJ7_HUMAN (SEQ ID NO:429)), referred to herein as the previously known protein.

The following GO Annotation(s) apply to the previously known protein. The following annotation(s) were found: catalytic activity, which are annotation(s) related to Molecular Function.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

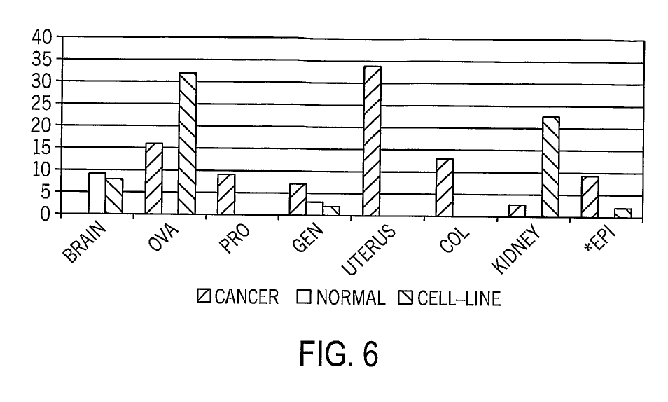

Cluster AA383349 can be used as a diagnostic marker according to overexpression of transcripts of this cluster in cancer. Expression of such transcripts in normal tissues is also given according to the previously described methods. The term “number” in the left hand column of the table and the numbers on the y-axis of FIG. 6 refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 6 and Table 5. This cluster is overexpressed (at least at a minimum level) in the following pathological conditions: epithelial malignant tumors.

TABLE 5
Normal tissue distribution

| Name of Tissue | Number |
|---|---|
| brain | 9 |
| ovary | 0 |
| prostate | 0 |
| general | 3 |
| uterus | 0 |
| colon | 0 |
| kidney | 0 |
| epithelial | 0 |

TABLE 6
P values and ratios for expression in cancerous tissue

| Name of Tissue | P1 | P2 | SP1 | R3 | SP2 | R4 |
|---|---|---|---|---|---|---|
| brain | 9.0e−01 | 8.9e−01 | 1.0e+00 | 0.2 | 8.4e−01 | 0.6 |
| ovary | 6.2e−01 | 4.2e−01 | 6.8e−01 | 1.5 | 5.9e−01 | 1.6 |
| prostate | 5.1e−01 | 5.8e−01 | 6.7e−01 | 1.5 | 7.5e−01 | 1.3 |
| general | 2.5e−01 | 4.4e−01 | 2.1e−01 | 1.5 | 4.4e−01 | 1.1 |
| uterus | 4.4e−01 | 6.2e−01 | 2.9e−01 | 2.0 | 5.1e−01 | 1.5 |
| colon | 3.5e−01 | 4.1e−01 | 6.9e−01 | 1.4 | 7.7e−01 | 1.3 |
| kidney | 5.7e−01 | 4.0e−01 | N/A | N/A | 7.0e−01 | 1.5 |
| epithelial | 1.9e−03 | 4.9e−03 | 2.2e−02 | 5.9 | 5.5e−02 | 4.3 |

As noted above, cluster AA383349 features 4 transcript(s), which were listed in Table 2 above. These transcript(s) encode for protein(s) which are variant(s) of protein Hypothetical protein (SEQ ID NO: 29). A description of each variant protein according to the present invention is now provided.

Variant protein AA383349_P4 (SEQ ID NO: 33) according to the present invention has an amino acid sequence as given in the “Sequence Listing” of the application; it is encoded by transcript(s) AA383349_T6 (SEQ ID NO: 334). An alignment is given to the known protein (Hypothetical protein, SEQ ID NOs:29-32, 429) in the alignment table, on the attached CDROM. One or more alignments to one or more previously published protein sequences are given in the alignment table, on the attached CDROM. A brief description of the relationship of the variant protein according to the present invention to each such aligned protein is as follows:

### 1. Comparison Report Between AA383349_P4 (SEQ ID NO: 33) and Q96EZ7_HUMAN (SEQ ID NO: 30):

A. An isolated chimeric polypeptide encoding for AA383349_P4 (SEQ ID NO: 33), comprising a amino acid sequence being at least about 90% homologous to MASRLLHRHIREQLKDLVEILQDPSPPPLCLPTTPGTPDSSDPSHLLGPQSCWSSQKEVSHESLVVGAIENAF QLMDEQMARERRGHQVEGGCCALVVIYLLGKVYVANAGDSRAIIVRNGEIIPMSREFTPETERQRLQLLG FLKPELLGSEFTHLEFPRRVLPKELGQRNLYRDQNMTGWAYKKIELEDLRFPLVCGEGKKARVMATIGVT RGLGDHSLKVCSSTLPIKPFLSCFPEVRVYDLTQYEHCPDDVLVLGTDGLWDVTTDCEVAATVDRVLSAY EPNDHSR corresponding to amino acids 151-440 of Q96EZ7_HUMAN (SEQ ID NO: 30), which also corresponds to amino acids 1-290 of AA383349_P4 (SEQ ID NO: 33).

### 2. Comparison Report Between AA383349_P4 (SEQ ID NO: 33) and NP_{—}005158 (SEQ ID NO: 31) (or Q9UF84_HUMAN (SEQ ID NO: 32)):

A. An isolated chimeric polypeptide encoding for AA383349_P4 (SEQ ID NO: 33), comprising a first amino acid sequence being at least about 90% homologous to MASRLLHRHIREQLKDL corresponding to amino acids 1-17 of NP_{—}005158 (SEQ ID NO: 31), which also corresponds to amino acids 1-17 of AA383349_P4 (SEQ ID NO: 33), a second amino acid sequence being at least about 70%, optionally at least 80%, preferably at least 85%, more preferably at least 90% and most preferably at least 95% homologous to a polypeptide having the sequence VEILQDPSPPPLCLPTTPGTPDSSDPSHLLGPQSCWSSQ (SEQ ID NO: 494) corresponding to amino acids 18-56 of AA383349_P4 (SEQ ID NO: 33), and a third amino acid sequence being at least about 90% homologous to KEVSHESLVVGAIENAFQLMDEQMARERRGHQVEGGCCALVVIYLLGKVYVANAGDSRAIIVRNGEIIPM SREFTPETERQRLQLLGFLKPELLGSEFTHLEFPRRVLPKELGQRMLYRDQNMTGWAYKKIELEDLRFPLV CGEGKKARVMATIGVTRGLGDHSLKVCSSTLPIKPFLSCFPEVRVYDLTQYEHCPDDVLVLGTDGLWDVT TDCEVAATVDRVLSAYEPNDHSR corresponding to amino acids 18-251 of NP_{—}005158 (SEQ ID NO: 31), which also corresponds to amino acids 57-290 of AA383349_P4 (SEQ ID NO: 33), wherein said first amino acid sequence, second amino acid sequence and third amino acid sequence are contiguous and in a sequential order.

B. An isolated polypeptide encoding for an edge portion of AA383349_P4 (SEQ ID NO: 33), comprising an amino acid sequence being at least about 70%, optionally at least about 80%, preferably at least about 85%, more preferably at least about 90% and most preferably at least about 95% homologous to the sequence VEILQDPSPPPLCLPTTPGTPDSSDPSHLLGPQSCWSSQ (SEQ ID NO: 494) of AA383349_P4 (SEQ ID NO: 33).

### 4. Comparison Report Between AA383349_P4 (SEQ ID NO: 33) and Q6DKJ7_HUMAN (SEQ ID NO:429):

A. An isolated chimeric polypeptide encoding for AA383349_P4 (SEQ ID NO: 33), comprising a amino acid sequence being at least about 90% homologous to MASRLLHRHIREQLKDLVEILQDPSPPPLCLPTTPGTPDSSDPSHLLGPQSCWSSQKEVSHESLVVGAIENAF QLMDEQMARERRGHQVEGGCCALVVIYLLGKVYVANAGDSRAIIVRNGEIIPMSREFTPETERQRLQLLG FLKPELLGSEFTHLEFPRRVLPKELGQRMLYRDQNMTGWAYKKIELEDLRFPLVCGEGKKARVMATIGVT RGLGDHSLKVCSSTLPIKPFLSCFPEVRVYDLTQYEHCPDDVLVLGTDGLWDVTTDCEVAATVDRVLSAY EPNDHSR corresponding to amino acids 168-457 of Q6DKJ7_HUMAN (SEQ ID NO:429), which also corresponds to amino acids 1-290 of AA383349_P4 (SEQ ID NO: 33), wherein said and first amino acid sequence are contiguous and in a sequential order.

The localization of the variant protein was determined according to results from a number of different software programs and analyses, including analyses from SignalP and other specialized programs. The variant protein is believed to be located intracellularly with regard to the cell.

Variant protein AA383349_P4 (SEQ ID NO: 33) also has the following non-silent SNPs (Single Nucleotide Polymorphisms) as listed in Table 7, (given according to their position(s) on the amino acid sequence, with the alternative amino acid(s) listed).

TABLE 7
Amino acid mutations

| SNP position(s) on amino acid | Alternative |
|---|---|
| sequence | amino acid(s) |
| 13 | Q -> * |
| 46 | L -> F |
| 75 | L -> P |
| 95 | A -> T |
| 124 | I -> T |

Variant protein AA383349_P4 (SEQ ID NO: 33) is encoded by the following transcript(s): AA383349_T6 (SEQ ID NO: 334), for which the coding portion starts at position 1582 and ends at position 2451. The transcript also has the following SNPs as listed in Table 8 (given according to their position on the nucleotide sequence, with the alternative nucleic acid listed).

TABLE 8
Nucleic acid SNPs

| | SNP position(s) on nucleotide |
|---|---|
| Polymorphism | sequence |
| G -> | 1521 |
| G -> T | 1566, 1719 |
| C -> T | 1618 |
| T -> C | 1805, 1952 |
| G -> A | 1864 |
| G -> A | 2001 |
| A -> G | 2494 |
| C -> | 2721 |
| -> A | 2778 |

Variant protein AA383349_P5 (SEQ ID NO: 34) according to the present invention has an amino acid sequence as given in the “Sequence Listing” of the application; it is encoded by transcript(s) AA383349_T7 (SEQ ID NO: 1). An alignment is given to the known protein (Hypothetical protein, SEQ ID NOs:29-32, 429) in the alignment table, on the attached CDROM. One or more alignments to one or more previously published protein sequences are given in the alignment table, on the attached CDROM. A brief description of the relationship of the variant protein according to the present invention to each such aligned protein is as follows:

### 1. Comparison Report Between AA383349_P5 (SEQ ID NO: 34) and Q6DKJ7_HUMAN (SEQ ID NO:429):

A. An isolated chimeric polypeptide encoding for AA383349_P5 (SEQ ID NO: 34), comprising a amino acid sequence being at least about 90% homologous to MLNRVRSAVAHLVSSGGAPPPRPKSPDLPNAASAPPAAAPEAPRSPPAKAGSGSATPAKAVEARASFSRPT FLQLSPGGLRRADDHAGRAVQSPPDTGRRLPWSTGYAEVINAGKSRHNEDQACCEVVYVEGRRSVTGVP REPSRGQGLCFYYWGLFDGHAGGGAAEMASRLLHRHIREQLKDLVEILQDPSPPPLCLPTTPGTPDSSDPS HLLGPQSCWSSQKEVSHESLVVGAIENAFQLMDEQMARERRGHQVEGGCCALVVIYLLGKVYVANAGD SRAIIVRNGEIIPMSREFTPETERQRLQLLGFLKPELLGSEFTHLEFPRRVLPKELGQRMLYRDQNMTGWAY KKIELEDLRFPLVCGEGKKARVMATIGVTRGLGDHSLKVCSSTLPIKPFLSCFPEVRVYDLTQYEHCPDDV LVLGTDGLWDVTTDCEVAATVDRVLSAYEPNDHSR corresponding to amino acids 1-457 of Q6DKJ7_HUMAN (SEQ ID NO:429), which also corresponds to amino acids 1-457 of AA383349_P5 (SEQ ID NO: 34).

### 2. Comparison Report Between AA383349_P5 (SEQ ID NO: 34) and Q96EZ7_HUMAN (SEQ ID NO: 30):

A. An isolated chimeric polypeptide encoding for AA383349_P5 (SEQ ID NO: 34), comprising a first amino acid sequence being at least about 70%, optionally at least 80%, preferably at least 85%, more preferably at least 90% and most preferably at least 95%, homologous to a polypeptide having the sequence MLNRVRSAVAHLVSSGG (SEQ ID NO: 495) corresponding to amino acids 1-17 of AA383349_P5 (SEQ ID NO: 34), and a second amino acid sequence being at least about 90% homologous to APPPRPKSPDLPNAASAPPAAAPEAPRSPPAKAGSGSATPAKAVEARASFSRPTFLQLSPGGLRRADDHAG RAVQSPPDTGRRLPWSTGYAEVINAGKSRHNEDQACCEVVYVEGRRSVTGVPREPSRGQGLCFYYWGLF DGHAGGGAAEMASRLLHRHIREQLKDLVEILQDPSPPPLCLPTTPGTPDSSDPSHLLGPQSCWSSQKEVSHE SLVVGAIENAFQLMDEQMARERRGHQVEGGCCALVVIYLLGKVYVANAGDSRAIIVRNGEIIPMSREFTPE TERQRLQLLGFLKPELLGSEFTHLEFPRRVLPKELGQRMLYRDQNMTGWAYKKIELEDLRFPLVCGEGKK ARVMATIGVTRGLGDHSLKVCSSTLPIKPFLSCFPEVRVYDLTQYEHCPDDVLVLGTDGLWDVTTDCEVA ATVDRVLSAYEPNDHSR corresponding to amino acids 1-440 of Q96EZ7_HUMAN (SEQ ID NO: 30), which also corresponds to amino acids 18-457 of AA383349_P5 (SEQ ID NO: 34), wherein said first amino acid sequence and second amino acid sequence are contiguous and in a sequential order.

B. An isolated polypeptide encoding for a head of AA383349_P5 (SEQ ID NO: 34), comprising a polypeptide being at least 70%, optionally at least about 80%, preferably at least about 85%, more preferably at least about 90% and most preferably at least about 95% homologous to the sequence MLNRVRSAVAHLVSSGG (SEQ ID NO: 495) of AA383349_P5 (SEQ ID NO: 34).

### 3. Comparison Report Between AA383349_P5 (SEQ ID NO: 34) and NP 005158 (SEQ ID NO: 31) (or Q9UF84_HUMAN (SEQ ID NO: 32)):

A. An isolated chimeric polypeptide encoding for AA383349_P5 (SEQ ID NO: 34), comprising a first amino acid sequence being at least about 70%, optionally at least 80%, preferably at least 85%, more preferably at least 90% and most preferably at least 95%, homologous to a polypeptide having the sequence MLNRVRSAVAHLVSSGGAPPPRPKSPDLPNAASAPPAAAPEAPRSPPAKAGSGSAJPAKAVEARASFSRPT FLQLSPGGLRRADDHAGRAVQSPPDTGRRLPWSTGYAEVINAGKSRHNEDQACCEVVYVEGRRSVTGVP REPSRGQGLCFYYWGLFDGHAGGGAAE (SEQ ID NO: 496) corresponding to amino acids 1-167 of AA383349_P5 (SEQ ID NO: 34), a second amino acid sequence being at least about 90% homologous to MASRLLHRHIREQLKDL corresponding to amino acids 1-17 of NP_{—}005158 (SEQ ID NO: 31), which also corresponds to amino acids 168-184 of AA383349_P5 (SEQ ID NO: 34), a third amino acid sequence being at least about 70%, optionally at least 80%, preferably at least 85%, more preferably at least 90% and most preferably at least 95% homologous to a polypeptide having the sequence VEILQDPSPPPLCLPTTPGTPDSSDPSHLLGPQSCWSSQ (SEQ ID NO: 494) corresponding to amino acids 185-223 of AA383349_P5 (SEQ ID NO: 34), and a fourth amino acid sequence being at least about 90% homologous to KEVSHESLVVGAIENAFQLMDEQMARERRGHQVEGGCCALVVIYLLGKVYVANAGDSRAIIVRNGEIIPM SREFTPETERQRLQLLGFLKPELLGSEFTHLEFPRRVLPKELGQRMLYRDQNMTGWAYKKIELEDLRFPLV CGEGKKARVMATIGVTRGLGDHSLKVCSSTLPIKPFLSCFPEVRVYDLTQYEHCPDDVLVLGTDGLWDVT TDCEVAATVDRVLSAYEPNDHSR corresponding to amino acids 18-251 of NP_{—}005158 (SEQ ID NO: 31), which also corresponds to amino acids 224-457 of AA383349_P5 (SEQ ID NO: 34), wherein said first amino acid sequence, second amino acid sequence, third amino acid sequence and fourth amino acid sequence are contiguous and in a sequential order.

B. An isolated polypeptide encoding for a head of AA383349_P5 (SEQ ID NO: 34), comprising a polypeptide being at least 70%, optionally at least about 80%, preferably at least about 85%, more preferably at least about 90% and most preferably at least about 95% homologous to the sequence

(SEQ ID NO: 496)

MLNRVRSAVAHLVSSGGAPPPRPKSPDLPNAASAPPAAAPEAPRSPPAKA
GSGSATPAKAVEARASFSRPTFLQLSPGGLRRADDHAGRAVQSPPDTGRR
LPWSTGYAEVINAGKSRHNEDQACCEVVYVEGRRSVTGVPREPSRGQGLC
FYYWGLFDGHAGGGAAE
of

(SEQ ID NO: 34)

AA383349_P5.

C. An isolated polypeptide encoding for an edge portion of AA383349_P5 (SEQ ID NO: 34), comprising an amino acid sequence being at least about 70%, optionally at least about 80%, preferably at least about 85%, more preferably at least about 90% and most preferably at least about 95% homologous to the sequence

(SEQ ID NO: 494)

VEILQDPSPPPLCLPTTPGTPDSSDPSHLLGPQSCWSSQ
of

(SEQ ID NO: 34)

AA383349_P5.

The localization of the variant protein was determined according to results from a number of different software programs and analyses, including analyses from SignalP and other specialized programs. The variant protein is believed to be located intracellularly with regard to the cell.

Variant protein AA383349_P5 (SEQ ID NO: 34) also has the following non-silent SNPs (Single Nucleotide Polymorphisms) as listed in Table 9, (given according to their position(s) on the amino acid sequence, with the alternative amino acid(s) listed).

TABLE 9
Amino acid mutations

| SNP position(s) on amino acid | Alternative |
|---|---|
| sequence | amino acid(s) |
| 147 | Q -> |
| 180 | Q -> * |
| 213 | L -> F |
| 242 | L -> P |
| 262 | A -> T |
| 291 | I -> T |

Variant protein AA383349_P5 (SEQ ID NO: 34) is encoded by the following transcript(s): AA383349_T7 (SEQ ID NO: 1), for which the coding portion starts at position 163 and ends at position 1533. The transcript also has the following SNPs as listed in Table 10 (given according to their position on the nucleotide sequence, with the alternative nucleic acid listed).

TABLE 10
Nucleic acid SNPs

| | SNP position(s) on nucleotide |
|---|---|
| Polymorphism | sequence |
| G -> | 603 |
| G -> T | 648, 801 |
| C -> T | 700 |
| T -> C | 887, 1034 |
| G -> A | 946, 1083 |
| A -> G | 1576 |
| C -> | 1803 |
| -> A | 1860 |

Variant protein AA383349_P11 (SEQ ID NO: 35) according to the present invention has an amino acid sequence as given in the “Sequence Listing” of the application; it is encoded by transcript(s) AA383349 T16 (SEQ ID NO: 2).

The localization of the variant protein was determined according to results from a number of different software programs and analyses, including analyses from SignalP and other specialized programs. The variant protein is believed to be located intracellularly with regard to the cell.

Variant protein AA383349_P11 (SEQ ID NO: 35) also has the following non-silent SNPs (Single Nucleotide Polymorphisms) as listed in Table 11, (given according to their position(s) on the amino acid sequence, with the alternative amino acid(s) listed).

TABLE 11
Amino acid mutations

| SNP position(s) on amino acid | |
|---|---|
| sequence | Alternative amino acid(s) |
| 13 | Q -> * |
| 46 | L -> F |
| 75 | L -> P |
| 95 | A -> T |
| 124 | I -> T |

Variant protein AA383349_P11 (SEQ ID NO: 35) is encoded by the following transcript(s): AA383349_T16 (SEQ ID NO: 2), for which the coding portion starts at position 1582 and ends at position 2133. The transcript also has the following SNPs as listed in Table 12 (given according to their position on the nucleotide sequence, with the alternative nucleic acid listed).

TABLE 12
Nucleic acid SNPs

| | SNP position(s) on nucleotide |
|---|---|
| Polymorphism | sequence |
| G -> | 1521 |
| G -> T | 1566, 1719 |
| C -> T | 1618 |
| T -> C | 1805, 1952 |
| G -> A | 1864, 2001 |
| A -> G | 2430 |
| C -> | 2657 |
| -> A | 2714 |

Variant protein AA383349_P12 (SEQ ID NO: 36) according to the present invention has an amino acid sequence as given in the “Sequence Listing” of the application; it is encoded by transcript(s) AA383349_T17 (SEQ ID NO: 3). An alignment is given to the known protein (Hypothetical protein, SEQ ID NOs: 29-32, 429) in the alignment table, on the attached CDROM. One or more alignments to one or more previously published protein sequences are given in the alignment table, on the attached CDROM. A brief description of the relationship of the variant protein according to the present invention to each such aligned protein is as follows:

### 1. Comparison Report Between AA383349_P12 (SEQ ID NO: 36) and Q6DKJ7_HUMAN (SEQ ID NO:429):

A. An isolated chimeric polypeptide encoding for AA383349_P12 (SEQ ID NO: 36), comprising a first amino acid sequence being at least about 90% homologous to MLNRVRSAVAHLVSSGGAPPPRPKSPDLPNAASAPPAAAPEAPRSPPAKAGSGSATPAKAVEARASFSRPT FLQLSPGGLRRADDHAGRAVQSPPDTGRRLPWSTGYAEVINAGKSRHNEDQACCEVVYVEGRRSVTGVP REPSRGQGLCFYYWGLFDGHAGGGAAEMASRLLHRHIREQLKDLVEILQDPSPPPLCLPTTPGTPDSSDPS HLLGPQSCWSSQKEVSBESLVVGAIENAFQLMDEQMARERRGHQVEGGCCALVVIYLLGKVYVANAGD SRAIIVRNGEIIPMSREFTPETERQRLQLLGFLKPELLGSEFTHLEFPRRVLPKELGQRMLYRDQNMTGW corresponding to amino acids 1-349 of Q6DKJ7_HUMAN (SEQ ID NO:429), which also corresponds to amino acids 1-349 of AA383349_P12 (SEQ ID NO: 36), and a second amino acid sequence LG corresponding to amino acids 350-351 of AA383349_P12 (SEQ ID NO: 36), wherein said first amino acid sequence and second amino acid sequence are contiguous and in a sequential order.

### 2. Comparison Report Between AA383349_P12 (SEQ ID NO: 36) and Q96EZ7_HUMAN (SEQ ID NO: 30):

A. An isolated chimeric polypeptide encoding for AA383349_P12 (SEQ ID NO: 36), comprising a first amino acid sequence being at least about 70%, optionally at least 80%, preferably at least 85%, more preferably at least 90% and most preferably at least 95%, homologous to a polypeptide having the sequence MLNRVRSAVAHLVSSGG (SEQ ID NO: 495) corresponding to amino acids 1-17 of AA383349_P12 (SEQ ID NO: 36), a second amino acid sequence being at least about 90% homologous to APPPRPKSPDLPNAASAPPAAAPEAPRSPPAKAGSGSATPAKAVEARASFSRPTFLQLSPGGLRRADDHAG RAVQSPPDTGRRLPWSTGYAEVINAGKSRHNEDQACCEVVYVEGRRSVTGVPREPSRGQGLCFYYWGLF DGHAGGGAAEMASRLLHRHIREQLKDLVEILQDPSPPPLCLPTTPGTPDSSDPSHLLGPQSCWSSQKEVSHE SLVVGAIENAFQLMDEQMARERRGHQVEGGCCALVVIYLLGKVYVANAGDSRAIIVRNGEIIPMSREFTPE TERQRLQLLGFLKPELLGSEFTHLEFPRRVLPKELGQRMLYRDQNMTGW corresponding to amino acids 1-332 of Q96EZ7_HUMAN (SEQ ID NO: 30), which also corresponds to amino acids 18-349 of AA383349_P12 (SEQ ID NO: 36), and a third amino acid sequence LG corresponding to amino acids 350-351 of AA383349_P12 (SEQ ID NO: 36), wherein said first amino acid sequence, second amino acid sequence and third amino acid sequence are contiguous and in a sequential order.

B. An isolated polypeptide encoding for a head of AA383349_P12 (SEQ ID NO: 36), comprising a polypeptide being at least 70%, optionally at least about 80%, preferably at least about 85%, more preferably at least about 90% and most preferably at least about 95% homologous to the sequence MLNRVRSAVAHLVSSGG (SEQ ID NO: 495) of AA383349_P12 (SEQ ID NO: 36).

The localization of the variant protein was determined according to results from a number of different software programs and analyses, including analyses from SignalP and other specialized programs. The variant protein is believed to be located intracellularly with regard to the cell.

Variant protein AA383349_P12 (SEQ ID NO: 36) also has the following non-silent SNPs (Single Nucleotide Polymorphisms) as listed in Table 13, (given according to their position(s) on the amino acid sequence, with the alternative amino acid(s) listed).

TABLE 13
Amino acid mutations

| SNP position(s) on amino acid | Alternative |
|---|---|
| sequence | amino acid(s) |
| 147 | Q -> |
| 180 | Q -> * |
| 213 | L -> F |
| 242 | L -> P |
| 262 | A -> T |
| 291 | I -> T |

Variant protein AA383349_P12 (SEQ ID NO: 36) is encoded by the following transcript(s): AA383349_T17 (SEQ ID NO: 3), for which the coding portion starts at position 163 and ends at position 1215. The transcript also has the following SNPs as listed in Table 14 (given according to their position on the nucleotide sequence, with the alternative nucleic acid listed).

TABLE 14
Nucleic acid SNPs

| | SNP position(s) on |
|---|---|
| Polymorphism | nucleotide sequence |
| G -> | 603 |
| G -> T | 648, 801 |
| C -> T | 700 |
| T -> C | 887, 1034 |
| G -> A | 946, 1083 |
| A -> G | 1512 |
| C -> | 1739 |
| -> A | 1796 |

As noted above, cluster AA383349 features 25 segment(s), which were listed in Table 3 above and for which the sequence(s) are given in the “Sequence Listing” of the application. These segment(s) are portions of nucleic acid sequence(s) which are described herein separately because they are of particular interest. A description of some of these segments according to the present invention is now provided.

Segment cluster AA383349_N32 (SEQ ID NO: 9) according to the present invention is supported by 7 libraries. The number of libraries was determined as previously described. This segment can be found in the following transcript(s): AA383349_T16 (SEQ ID NO: 2), AA383349_T17 (SEQ ID NO: 3), AA383349_T6 (SEQ ID NO: 334) and AA383349_T7 (SEQ ID NO: 1). Table 15 below describes the starting and ending position of this segment on each transcript.

TABLE 15
Segment location on transcripts

| | Segment | Segment |
|---|---|---|
| Transcript name | starting position | ending position |
| AA383349_T16 (SEQ ID NO: 2) | 2387 | 2528 |
| AA383349_T17 (SEQ ID NO: 3) | 1469 | 1610 |
| AA383349_T6 (SEQ ID NO: 334) | 2451 | 2592 |
| AA383349_T7 (SEQ ID NO: 1) | 1533 | 1674 |

Segment cluster AA383349_N34 (SEQ ID NO: 10) according to the present invention is supported by 52 libraries. The number of libraries was determined as previously described. This segment can be found in the following transcript(s): AA383349_T16 (SEQ ID NO: 2), AA383349_T17 (SEQ ID NO: 3), AA383349_T6 (SEQ ID NO: 334) and AA383349_T7 (SEQ ID NO: 1). Table 16 below describes the starting and ending position of this segment on each transcript.

TABLE 16
Segment location on transcripts

| | Segment | Segment |
|---|---|---|
| Transcript name | starting position | ending position |
| AA383349_T16 (SEQ ID NO: 2) | 2536 | 2773 |
| AA383349_T17 (SEQ ID NO: 3) | 1618 | 1855 |
| AA383349_T6 (SEQ ID NO: 334) | 2600 | 2837 |
| AA383349_T7 (SEQ ID NO: 1) | 1682 | 1919 |

According to an optional embodiment of the present invention, short segments related to the above cluster are also provided. These segments are up to about 120 bp in length, and so are included in a separate description.

Segment cluster AA383349_N23 (SEQ ID NO: 21) according to the present invention is supported by 56 libraries. The number of libraries was determined as previously described. This segment can be found in the following transcript(s): AA383349_T16 (SEQ ID NO: 2), AA383349_T17 (SEQ ID NO: 3), AA383349_T6 (SEQ ID NO: 334) and AA383349_T7 (SEQ ID NO: 1). Table 17 below describes the starting and ending position of this segment on each transcript.

TABLE 17
Segment location on transcripts

| | Segment | Segment |
|---|---|---|
| Transcript name | starting position | ending position |
| AA383349_T16 (SEQ ID NO: 2) | 2008 | 2126 |
| AA383349_T17 (SEQ ID NO: 3) | 1090 | 1208 |
| AA383349_T6 (SEQ ID NO: 334) | 2008 | 2126 |
| AA383349_T7 (SEQ ID NO: 1) | 1090 | 1208 |

Segment cluster AA383349_N27 (SEQ ID NO: 23) according to the present invention is supported by 53 libraries. The number of libraries was determined as previously described. This segment can be found in the following transcript(s): AA383349_T16 (SEQ ID NO: 2), AA383349_T17 (SEQ ID NO: 3), AA383349_T6 (SEQ ID NO: 334) and AA383349_T7 (SEQ ID NO: 1). Table 18 below describes the starting and ending position of this segment on each transcript.

TABLE 18
Segment location on transcripts

| | Segment | Segment |
|---|---|---|
| Transcript name | starting position | ending position |
| AA383349_T16 (SEQ ID NO: 2) | 2127 | 2234 |
| AA383349_T17 (SEQ ID NO: 3) | 1209 | 1316 |
| AA383349_T6 (SEQ ID NO: 334) | 2191 | 2298 |
| AA383349_T7 (SEQ ID NO: 1) | 1273 | 1380 |

Segment cluster AA383349_N29 (SEQ ID NO: 24) according to the present invention is supported by 49 libraries. The number of libraries was determined as previously described. This segment can be found in the following transcript(s): AA383349_T16 (SEQ ID NO: 2), AA383349_T17 (SEQ ID NO: 3), AA383349_T6 (SEQ ID NO: 334) and AA383349_T7 (SEQ ID NO: 1). Table 19 below describes the starting and ending position of this segment on each transcript.

TABLE 19
Segment location on transcripts

| | Segment | Segment |
|---|---|---|
| Transcript name | starting position | ending position |
| AA383349_T16 (SEQ ID NO: 2) | 2235 | 2249 |
| AA383349_T17 (SEQ ID NO: 3) | 1317 | 1331 |
| AA383349_T6 (SEQ ID NO: 334) | 2299 | 2313 |
| AA383349_T7 (SEQ ID NO: 1) | 1381 | 1395 |

Segment cluster AA383349 N30 (SEQ ID NO: 25) according to the present invention is supported by 53 libraries. The number of libraries was determined as previously described. This segment can be found in the following transcript(s): AA383349_T16 (SEQ ID NO: 2), AA383349_T17 (SEQ ID NO: 3), AA383349_T6 (SEQ ID NO: 334) and AA383349_T7 (SEQ ID NO: 1). Table 20 below describes the starting and ending position of this segment on each transcript.

TABLE 20
Segment location on transcripts

| | Segment | Segment |
|---|---|---|
| Transcript name | starting position | ending position |
| AA383349_T16 (SEQ ID NO: 2) | 2250 | 2330 |
| AA383349_T17 (SEQ ID NO: 3) | 1332 | 1412 |
| AA383349_T6 (SEQ ID NO: 334) | 2314 | 2394 |
| AA383349_T7 (SEQ ID NO: 1) | 1396 | 1476 |

Microarray (chip) data is available for segments AA383349_N27 (SEQ ID NO: 23), AA383349_N29 (SEQ ID NO: 24) and AA383349_N30 (SEQ ID NO: 25), as follows. As described above with regard to the cluster itself, various oligonucleotides were tested for being differentially expressed in various disease conditions, particularly cancer. The following oligonucleotides were found to hit this segment, shown in Table 21.

TABLE 21
Oligonucleotides related to this segment

| | | Chip |
|---|---|---|
| Oligonucleotide name | Overexpressed in cancers | reference |
| AA383349_0_8_0 (SEQ ID NO: | breast malignant tumors | BRS |
| 528) | | |

Expression of Homo sapiens protein phosphatase 2a, catalytic subunit, zeta isoform (PPP2CZ) transcripts detectable by or according to seg34WT-AA383349_seg34WT (SEQ ID NO: 358) amplicon and primers AA383349_seg34WTF (SEQ ID NO: 356) and AA383349_seg34WTR (SEQ ID NO: 357) or of PPP2CZ transcripts detectable by or according to seg32—AA383349_seg32 (SEQ ID NO: 361) amplicon and primers AA383349_seg32F (SEQ ID NO: 359) and AA383349_seg32R (SEQ ID NO: 360) was measured by real time PCR.

The sequences of corresponding primers and amplicons are given below.

Forward Primer AA383349_seg34WTF (SEQ ID NO: 356):
TCTCCCCAACAACAAGCTGG
Reverse Primer AA383349_seg34WTR (SEQ ID NO: 357):
GAGAGGCTAGTGGGAGGGATG
Amplicon AA383349_seg34WT (SEQ ID NO: 358):
TCTCCCCAACAACAAGCTGGGTTCCGGGGATGACATCTCTGTCTTCGTCA
TCCCCCTGGGAGGGCCAGGCAGTTACTCCTGAGGGGCTGAACACCATCCC
TCCCACTAGCCTCTC
Forward Primer AA383349_seg32F (SEQ ID NO: 359):
AGGGGCTGCATGGCATG
Reverse Primer AA383349_seg32R (SEQ ID NO: 360):
TATGTGGGAAGACAGATACACAGGC
Amplicon AA383349_seg32 (SEQ ID NO: 361):
AGGGGCTGCATGGCATGGTGGTAAGGGGATGGCATTGGTGAAAGAAGGGT
CAAAGGGAAGCAATGCTTGGGACCCCACATGTGGGCCTGTGTATCTGTCT
TCCCACATAC

Expression of Homo sapiens Protein Phosphatase 2a, Catalytic Subunit, Zeta Isoform (PPP2CZ) AA383349 Transcripts which are Detectable by Amplicon as Depicted in Sequence Name AA383349_Seg34WT (SEQ ID NO: 358) in Normal and Cancerous Breast Tissues

Expression of Homo sapiens protein phosphatase 2a, catalytic subunit, zeta isoform (PPP2CZ) transcripts detectable by or according to seg34WT-AA383349_seg34WT (SEQ ID NO: 358) amplicon and primers AA383349_seg34WTF (SEQ ID NO: 356) and AA383349_seg34WTR (SEQ ID NO: 357) was measured by real time PCR. In parallel the expression of four housekeeping genes—G6PD (GenBank Accession No. NM_{—}000402 (SEQ ID NO:339); G6PD amplicon (SEQ ID NO: 311)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 57, 59, 60, 63, 66, 64, 56, 65, 67 and 58, Table 1_{—}3 above), to obtain a value of fold up-regulation for each sample relative to the median of the normal PM samples.

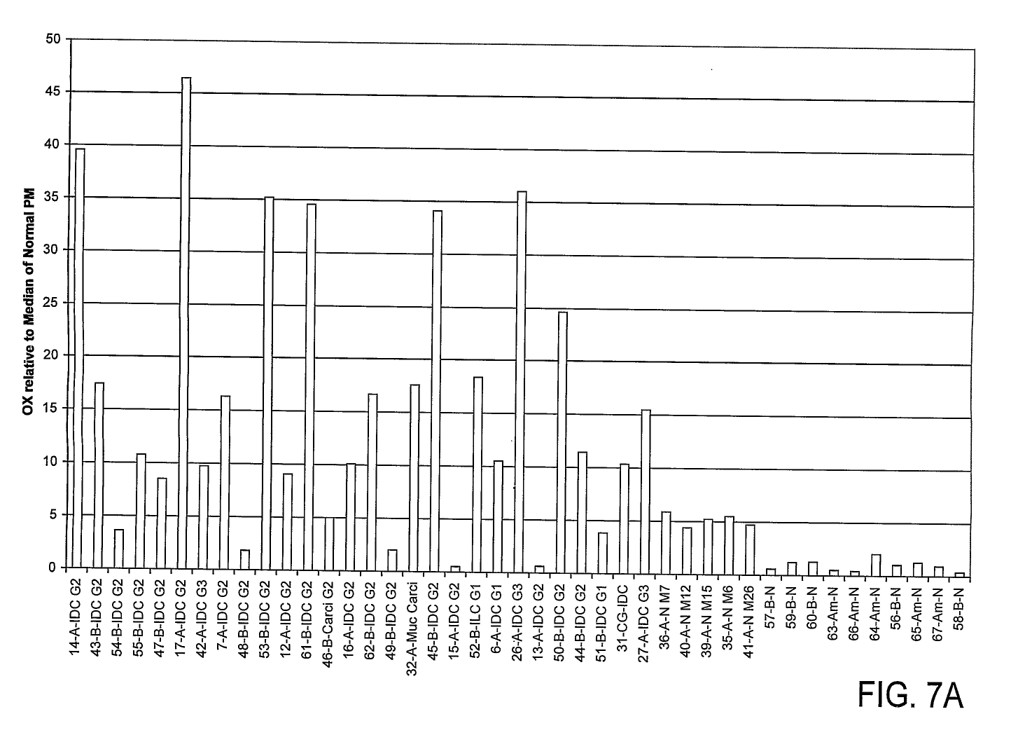

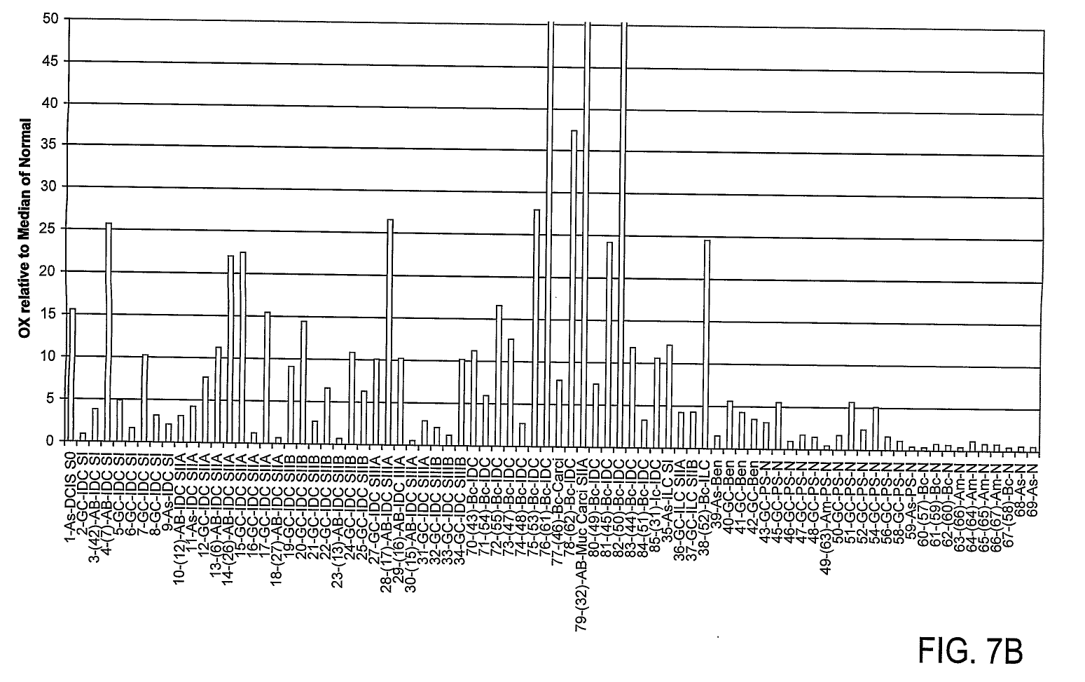

FIG. 7A is a histogram showing over expression of the Homo sapiens protein phosphatase 2a, catalytic subunit, zeta isoform (PPP2CZ) transcripts for transcripts detectable by or according to seg34WT-AA383349 (SEQ ID NO: 358) in cancerous Breast samples relative to the normal samples.

As is evident from FIG. 7A, the expression of Homo sapiens protein phosphatase 2a, catalytic subunit, zeta isoform (PPP2CZ) transcripts detectable by the above amplicon in cancer samples was significantly higher than in the non-cancerous samples (sample numbers 57, 59, 60, 63, 66, 64, 56, 65, 67 and 58, Table 1_{—}3 above). Notably an over-expression of at least 5 fold was found in 22 out of 28 adenocarcinoma samples.

Statistical analysis was applied to verify the significance of these results, as described below.

The P value for the difference in the expression levels of Homo sapiens protein phosphatase 2a, catalytic subunit, zeta isoform (PPP2CZ) transcripts detectable by the above amplicon in Breast cancer samples versus the normal tissue samples was determined by T test as 1.52e-06.

Threshold of 5 fold over expression was found to differentiate between cancer and normal samples with P value of 1.69e-05 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: AA383349_seg34WTF (SEQ ID NO: 356) forward primer; and AA383349_seg34WTR (SEQ ID NO: 357) reverse primer.

The present invention also preferably encompasses any amplicon obtained through the use of any suitable primer pair; for example, for the above experiment, the following amplicon was obtained as a non-limiting illustrative example only of a suitable amplicon: AA383349_seg34WT (SEQ ID NO: 358).

Expression of Homo sapiens protein phosphatase 2a, catalytic subunit, zeta isoform (PPP2CZ) transcripts detectable by or according to seg34WT-AA383349_seg34WT (SEQ ID NO: 358) amplicon and primers AA383349_seg34WTF (SEQ ID NO: 356) and AA383349_seg34WTR (SEQ ID NO: 357) was further measured by real time PCR using enlarged panel as shown in Table 1_{—}7. In parallel the expression of four housekeeping genes—G6PD (GenBank Accession No. NM_{—}000402 (SEQ ID NO:339); G6PD amplicon (SEQ ID NO: 311)), RPL19 (GenBank Accession No. NM_{—}000981 (SEQ ID NO:342); RPL19 amplicon (SEQ ID NO: 320)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 43, 45, 46, 47, 48, 49, 50, 51, 52, 54, 56, 58, 59, 60, 61, 62, 63, 64, 65, 66, 67, 68 and 69, Table 1_{—}7 above), to obtain a value of fold up-regulation for each sample relative to the median of the normal samples.

FIG. 7B is a histogram showing over expression of the above-indicated Homo sapiens protein phosphatase 2a, catalytic subunit, zeta isoform (PPP2CZ) transcripts in cancerous Breast samples relative to the normal samples.

As is evident from FIG. 7B, the expression of Homo sapiens protein phosphatase 2a, catalytic subunit, zeta isoform (PPP2CZ) transcripts detectable by the above amplicon in cancer samples was significantly higher than in the non-cancerous samples (sample numbers 43, 45, 46, 47, 48, 49, 50, 51, 52, 54, 56, 58, 59, 60, 61, 62, 63, 64, 65, 66, 67, 68 and 69, Table 1_{—}7 above). Notably an over-expression of at least 5 fold was found in 33 out of 53 adenocarcinoma samples.

Statistical analysis was applied to verify the significance of these results, as described below.

The P value for the difference in the expression levels of Homo sapiens protein phosphatase 2a, catalytic subunit, zeta isoform (PPP2CZ) transcripts detectable by the above amplicon in Breast cancer samples versus the normal tissue samples was determined by T test as 1.63e-002.

Threshold of 5 fold over expression was found to differentiate between cancer and normal samples with P value of 6.25e-005 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: AA383349_seg34WTF (SEQ ID NO: 356) forward primer; and AA383349_seg34WTR (SEQ ID NO: 357) reverse primer.

The present invention also preferably encompasses any amplicon obtained through the use of any suitable primer pair; for example, for the above experiment, the following amplicon was obtained as a non-limiting illustrative example only of a suitable amplicon: AA383349_seg34WT (SEQ ID NO: 358).

Expression of Homo sapiens Protein Phosphatase 2a, Catalytic Subunit, Zeta Isoform (PPP2CZ) AA383349 Transcripts which are Detectable by Amplicon as Depicted in Sequence Name AA383349_seg34WT (SEQ ID NO: 358) in Different Normal Tissues

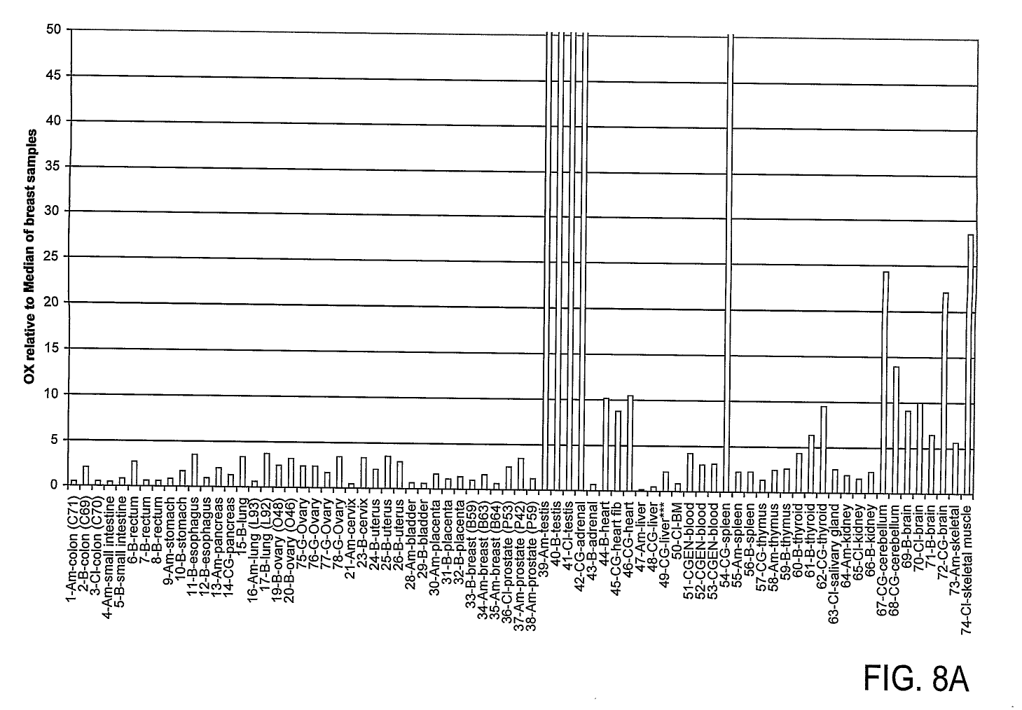

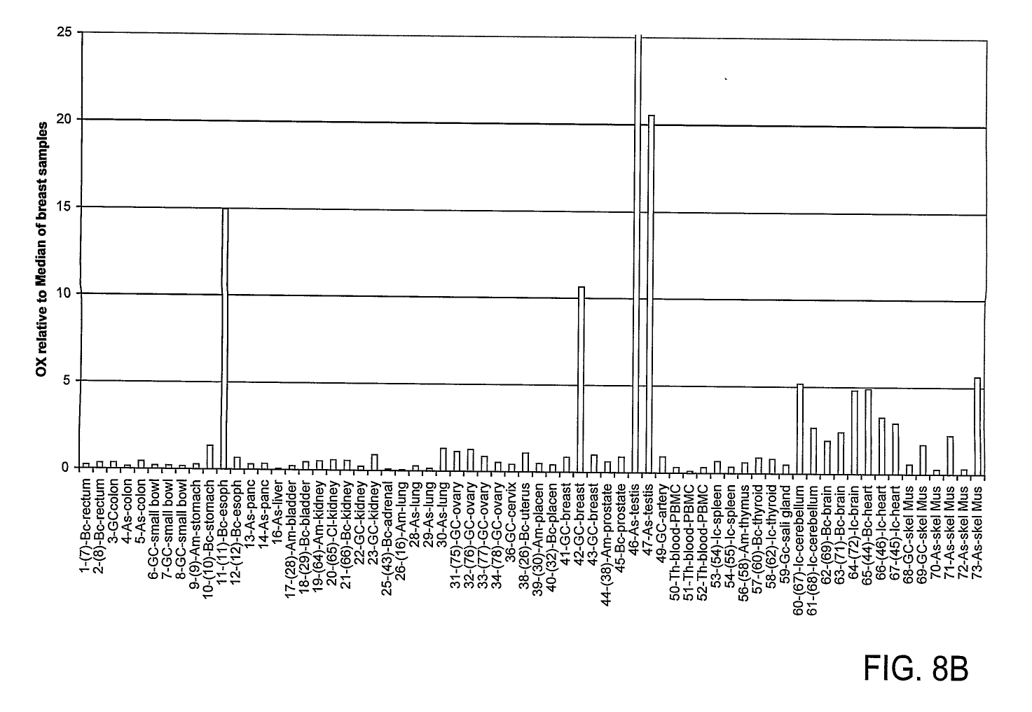

Expression of Homo sapiens protein phosphatase 2a, catalytic subunit, zeta isoform (PPP2CZ) transcripts detectable by or according to seg34WT-AA383349_seg34WT (SEQ ID NO: 358) amplicon and primers AA383349_seg34WTF (SEQ ID NO: 356) and AA383349_seg34WTR (SEQ ID NO: 357) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon-Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the geometric mean of the quantities of the housekeeping genes. The normalized quantity of each RT sample was then divided by the median of the quantities of the breast samples (sample numbers 33, 34 and 35, Table 1_{—}9 above), to obtain a value of relative expression of each sample relative to the median of the breast samples. FIG. 8A shows the results of real time PCR measurement of expression of transcripts detectable by or according to AA383349_seg34WT (SEQ ID NO: 358).

Expression of Homo sapiens protein phosphatase 2a, catalytic subunit, zeta isoform (PPP2CZ) transcripts detectable by or according to seg34WT-AA383349_seg34WT (SEQ ID NO: 358) amplicon and primers AA383349_seg34WTF (SEQ ID NO: 356) and AA383349_seg34WTR (SEQ ID NO: 357) was further measured by real time PCR using updated panel as shown in Table 1_{—}10. In parallel the expression of three housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)) and TATA box (GenBank Accession No. NM_{—}003194 (SEQ ID NO:343); TATA amplicon (SEQ ID NO: 323)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the breast samples (sample numbers 41, 42 and 43, Table 1_{—}10 above), to obtain a value of relative expression of each sample relative to the median of the breast samples. FIG. 8B shows the results of real time PCR measurement of expression of transcripts detectable by or according to AA383349_seg34WT (SEQ ID NO: 358).

Expression of Protein Phosphatase 2a AA383349 Transcripts which are Detectable by Amplicon as Depicted in Sequence Name AA383349_seg32 (SEQ ID NO: 361) in Normal and Cancerous Breast Tissues.

Expression of protein phosphatase 2a transcripts detectable by or according to seg32—AA383349_seg32 (SEQ ID NO: 361) amplicon and primers AA383349_seg32F (SEQ ID NO: 359) and AA383349_seg32R (SEQ ID NO: 360) was measured by real time PCR. In parallel the expression of four housekeeping genes-G6PD (GenBank Accession No. NM_{—}000402 (SEQ ID NO:339); G6PD amplicon (SEQ ID NO: 311)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302) and SDHA (GenBank Accession No. NM_{—}004168 (SEQ ID NO:335); amplicon—SDHA-amplicon (SEQ ID NO:299) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 57, 59, 60, 63, 66, 64, 56, 65, 67 and 58, Table 1_{—}3 above: “Tissue samples in breast cancer testing panel”), to obtain a value of fold up-regulation for each sample relative to the median of the normal PM samples.

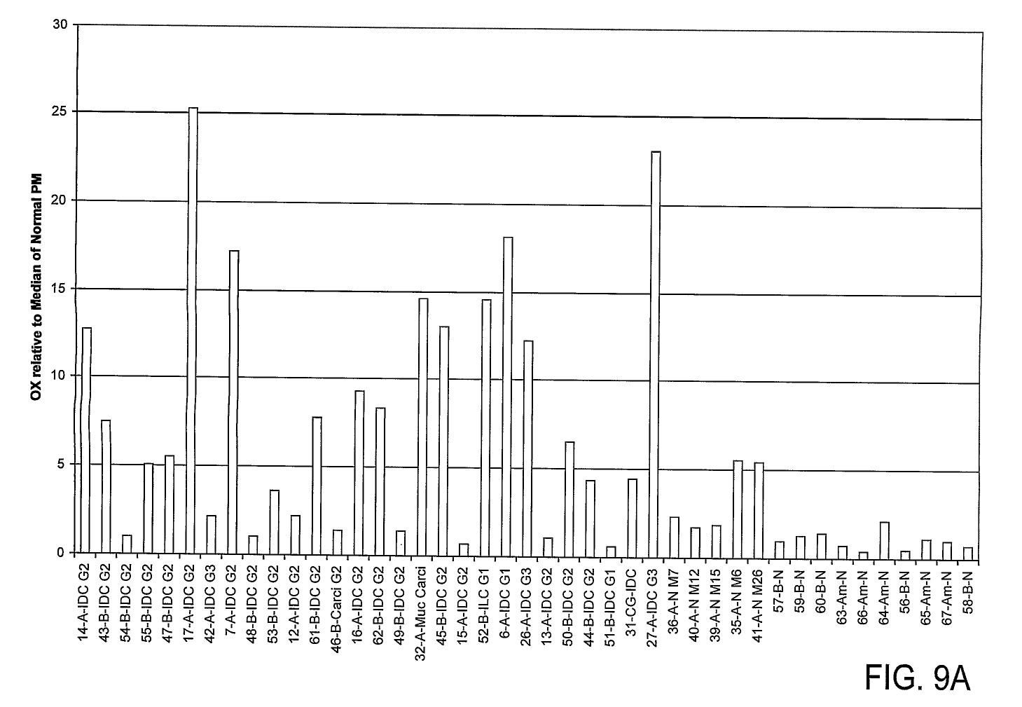

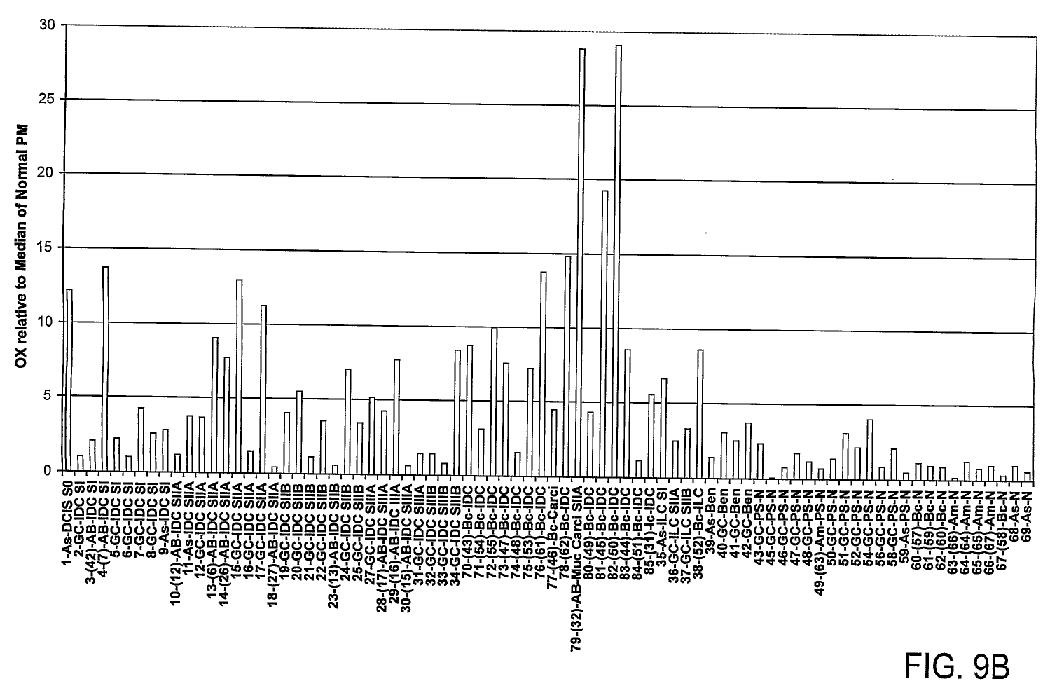

FIG. 9A is a histogram showing over expression of the protein phosphatase 2a transcripts in cancerous Breast samples relative to the normal samples.

As is evident from FIG. 9A, the expression of protein phosphatase 2a transcripts detectable by the above amplicon in cancer samples was significantly higher than in the non-cancerous samples (sample numbers 57, 59, 60, 63, 66, 64, 56, 65, 67 and 58, Table 1_{—}3 above, “Tissue samples in breast cancer testing panel”). Notably an over-expression of at least 5 fold was found in 16 out of 28 adenocarcinoma samples.

Statistical analysis was applied to verify the significance of these results, as described below.

The P value for the difference in the expression levels of protein phosphatase 2a transcripts detectable by the above amplicon in Breast cancer samples versus the normal tissue samples was determined by T test as 1.56e-005.

Threshold of 5 fold over expression was found to differentiate between cancer and normal samples with P value of 1.37e-003 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: AA383349_seg32F (SEQ ID NO: 359) forward primer; and AA383349_seg32R (SEQ ID NO: 360) (reverse primer.

The present invention also preferably encompasses any amplicon obtained through the use of any suitable primer pair; for example, for the above experiment, the following amplicon was obtained as a non-limiting illustrative example only of a suitable amplicon: AA383349_seg32 (SEQ ID NO: 361).

Expression of Homo sapiens protein phosphatase 2a, catalytic subunit, zeta isoform (PPP2CZ) transcripts detectable by or according to seg32—AA383349_seg32 (SEQ ID NO: 361) amplicon and primers AA383349_seg32F (SEQ ID NO: 359) and AA383349_seg32R (SEQ ID NO: 360) was further measured by real time PCR using enlarged panel as shown in Table 1_{—}7. In parallel the expression of four housekeeping genes—G6PD (GenBank Accession No. NM_{—}000402 (SEQ ID NO:339); G6PD amplicon (SEQ ID NO: 311)), RPL19 (GenBank Accession No. NM_{—}000981 (SEQ ID NO:342); RPL19 amplicon (SEQ ID NO: 320)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 43, 45, 46, 47, 48, 49, 50, 51, 52, 54, 56, 58, 59, 60, 61, 62, 63, 64, 65, 66, 67, 68 and 69, Table 1_{—}7 above), to obtain a value of fold up-regulation for each sample relative to the median of the normal samples.

FIG. 9B is a histogram showing over expression of the above-indicated Homo sapiens protein phosphatase 2a, catalytic subunit, zeta isoform (PPP2CZ) transcripts in cancerous Breast samples relative to the normal samples.

As is evident from FIG. 9B, the expression of Homo sapiens protein phosphatase 2a, catalytic subunit, zeta isoform (PPP2CZ) transcripts detectable by the above amplicon in cancer samples was significantly higher than in the non-cancerous samples (sample numbers 43, 45, 46, 47, 48, 49, 50, 51, 52, 54, 56, 58, 59, 60, 61, 62, 63, 64, 65, 66, 67, 68 and 69, Table 1_{—}7 above). Notably an over-expression of at least 5 fold was found in 24 out of 53 adenocarcinoma samples.

Statistical analysis was applied to verify the significance of these results, as described below.

The P value for the difference in the expression levels of Homo sapiens protein phosphatase 2a, catalytic subunit, zeta isoform (PPP2CZ) transcripts detectable by the above amplicon in Breast cancer samples versus the normal tissue samples was determined by T test as 3.19e-007.

Threshold of 5 fold over expression was found to differentiate between cancer and normal samples with P value of 2.07e-005 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: AA383349_seg32F (SEQ ID NO: 359) forward primer; and AA383349_seg32R (SEQ ID NO: 360) reverse primer.

The present invention also preferably encompasses any amplicon obtained through the use of any suitable primer pair; for example, for the above experiment, the following amplicon was obtained as a non-limiting illustrative example only of a suitable amplicon: AA383349_seg32 (SEQ ID NO: 361).

Expression of Protein Phosphatase 2a AA383349 Transcripts which are Detectable by Amplicon as Depicted in Sequence Name AA383349_seg32 (SEQ ID NO: 361) in Different Normal Tissues.

Expression of protein phosphatase 2a transcripts detectable by or according to seg32—AA383349_seg32 (SEQ ID NO: 361) amplicon and primers AA383349_seg32F (SEQ ID NO: 359) and AA383349_seg32R (SEQ ID NO: 360) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the geometric mean of the quantities of the housekeeping genes. The normalized quantity of each RT sample was then divided by the median of the quantities of the breast samples (sample numbers 33, 34 and 35, Table 1_{—}9 above: “Tissue samples in normal panel”), to obtain a value of relative expression of each sample relative to the median of the breast samples.

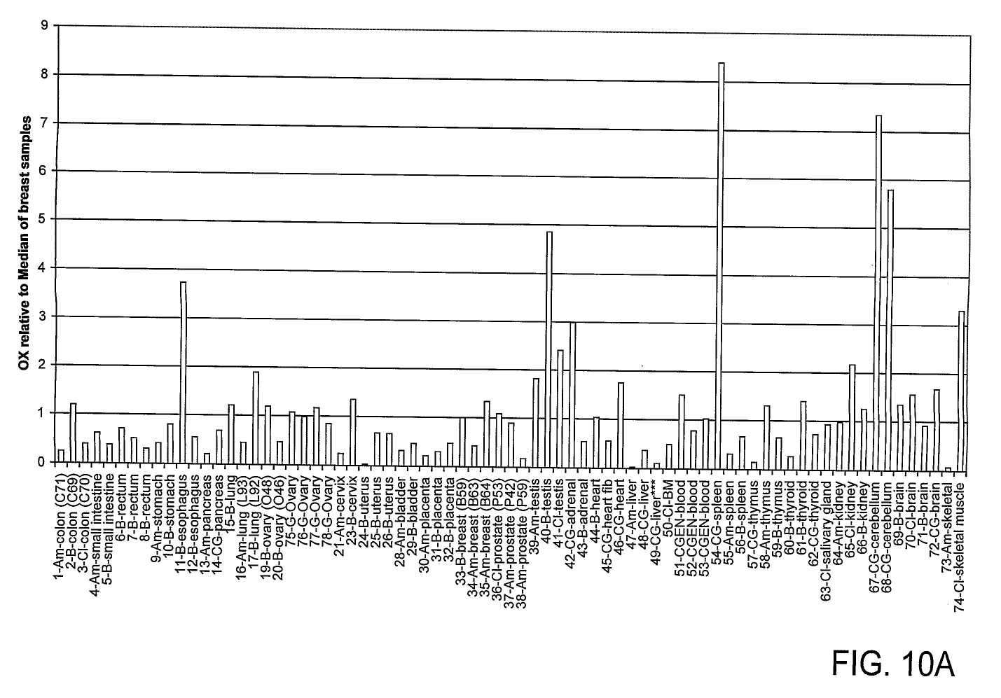

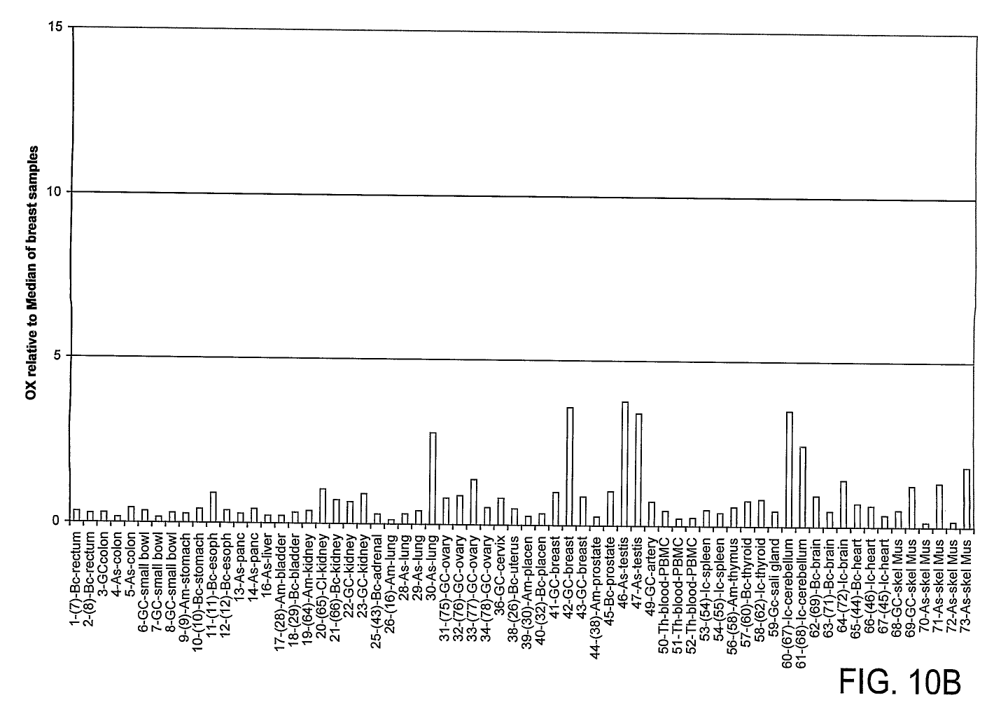

FIG. 10A is a histogram showing Expression of protein phosphatase 2a AA383349 transcripts which are detectable by amplicon as depicted in sequence name AA383349_seg32 (SEQ ID NO: 361) in different normal tissues.

Expression of Homo sapiens protein phosphatase 2a, catalytic subunit, zeta isoform (PPP2CZ) transcripts detectable by or according to seg32—AA383349_seg32 (SEQ ID NO: 361) amplicon and primers AA383349_seg32F (SEQ ID NO: 359) and AA383349_seg32R (SEQ ID NO: 360) was further measured by real time PCR using updated panel as shown in Table 1_{—}10. In parallel the expression of three housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)) and TATA box (GenBank Accession No. NM_{—}003194 (SEQ ID NO:343); TATA amplicon (SEQ ID NO: 323)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the breast samples (sample numbers 41, 42 and 43, Table 1_{—}10 above), to obtain a value of relative expression of each sample relative to the median of the breast samples.

FIG. 10B is a histogram showing Expression of protein phosphatase 2a AA383349 transcripts which are detectable by amplicon as depicted in sequence name AA383349_seg32 (SEQ ID NO: 361) in different normal tissues.

Expression of Homo sapiens Protein Phosphatase 2a, Catalytic Subunit, Zeta Isoform (PPP2CZ) AA383349 Transcripts which are Detectable by Amplicon as Depicted in Sequence Name AA383349_Seg32 (SEQ ID NO: 361) in Normal, Benign and Cancerous Ovary Tissues

Expression of Homo sapiens protein phosphatase 2a, catalytic subunit, zeta isoform (PPP2CZ) transcripts detectable by or according to seg32, AA383349_seg32 (SEQ ID NO: 361) amplicon and primers AA383349_seg32F (SEQ ID NO: 359) AA383349_seg32R (SEQ ID NO: 360) was measured by real time PCR. In parallel the expression of three housekeeping genes—HPRT1 (GenBank Accession No. NM_{—}000194 (SEQ ID NO:337); amplicon—HPRT1-amplicon (SEQ ID NO: 305) (SEQ ID NO: 305)), G6PD (GenBank Accession No. NM_{—}000402 (SEQ ID NO:339); G6PD amplicon (SEQ ID NO: 311)) and SDHA (GenBank Accession No. NM_{—}004168 (SEQ ID NO:335); amplicon—SDHA-amplicon (SEQ ID NO:299)) was measured similarly For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (Sample Nos 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5, above), to obtain a value of fold differential expression for each sample relative to the median of the normal samples.

In one experiment that was carried out no differential expression in the cancerous samples relative to the normal samples was observed.

Primer pairs are also optionally and preferably encompassed within the present invention; for example, for the above experiment, the following primer pair was used as a non-limiting illustrative example only of a suitable primer pair: AA383349_seg32F (SEQ ID NO: 359) forward primer; and AA383349_seg32R (SEQ ID NO: 360) reverse primer.

The present invention also preferably encompasses any amplicon obtained through the use of any suitable primer pair; for example, for the above experiment, the following amplicon was obtained as a non-limiting illustrative example only of a suitable amplicon: AA383349_seg32 (SEQ ID NO: 361).

Expression of Homo sapiens Protein Phosphatase 2a, Catalytic Subunit, Zeta Isoform (PPP2CZ) AA383349 Transcripts which are Detectable by Amplicon as Depicted in Sequence Name AA383349_Seg32 (SEQ ID NO: 361) in Normal and Cancerous Lung Tissues

Expression of Homo sapiens protein phosphatase 2a, catalytic subunit, zeta isoform (PP2CZ) transcripts detectable by or according to seg32, AA383349_seg32 (SEQ ID NO: 361) amplicon and primers AA383349_seg32F (SEQ ID NO: 359) AA383349_seg32R (SEQ ID NO: 360) was measured by real time PCR. In parallel the expression of four housekeeping genes—PBGD (GenBank Accession No. BC019323 (SEQ ID NO:336); amplicon—PBGD-amplicon (SEQ ID NO: 302)), HPRT1 (GenBank Accession No. NM_{—}000194 (SEQ ID NO:337); amplicon—HPRT1-amplicon (SEQ ID NO: 305) (SEQ ID NO: 305)), Ubiquitin (GenBank Accession No. BC000449 (SEQ ID NO:341); amplicon—Ubiquitin-amplicon (SEQ ID NO: 317)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (Sample Nos 51, 52, 53, 54, 55, 56, 57, 58, 59, 60, 61, 62, 63, 64, 69 and 70, Table 1_{—}6, above), to obtain a value of fold differential expression for each sample relative to the median of the normal samples.

In one experiment that was carried out no differential expression in the cancerous samples relative to the normal samples was observed.

Primer pairs are also optionally and preferably encompassed within the present invention; for example, for the above experiment, the following primer pair was used as a non-limiting illustrative example only of a suitable primer pair: AA383349_seg32F (SEQ ID NO: 359) forward primer; and AA383349_seg32R (SEQ ID NO: 360) reverse primer.

The present invention also preferably encompasses any amplicon obtained through the use of any suitable primer pair; for example, for the above experiment, the following amplicon was obtained as a non-limiting illustrative example only of a suitable amplicon: AA383349_seg32 (SEQ ID NO: 361).

Expression of Homo sapiens protein phosphatase 2a, catalytic subunit, zeta isoform (PPP2CZ) AA383349 transcripts which are detectable by amplicon as depicted in sequence name AA383349_seg32 (SEQ ID NO: 361) in normal and cancerous colon tissues

Expression of Homo sapiens protein phosphatase 2a, catalytic subunit, zeta isoform (PPP2CZ) transcripts detectable by or according to seg32, AA383349_seg32 (SEQ ID NO: 361) amplicon and primers AA383349_seg32F (SEQ ID NO: 359) AA383349_seg32R (SEQ ID NO: 360) was measured by real time PCR. In parallel the expression of three housekeeping genes—PBGD (GenBank Accession No. BC019323 (SEQ ID NO:336); amplicon—PBGD-amplicon (SEQ ID NO: 302)), BPRT1 (GenBank Accession No. NM_{—}000194 (SEQ ID NO:337); amplicon—HPRTI-amplicon (SEQ ID NO: 305) (SEQ ID NO: 305)), G6PD (GenBank Accession No. NM_{—}000402 (SEQ ID NO:339); G6PD amplicon (SEQ ID NO: 311)), was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (Sample Nos52, 42, 43, 44, 45, 46, 47, 48, 49, 50, 51, 53, 54, 55, 56, 57, 58, 59, 60, 61, 62, 63 and 64, Table 1_{—}8, above), to obtain a value of fold differential expression for each sample relative to the median of the normal samples.

In one experiment that was carried out no differential expression in the cancerous samples relative to the normal samples was observed.

Primer pairs are also optionally and preferably encompassed within the present invention; for example, for the above experiment, the following primer pair was used as a non-limiting illustrative example only of a suitable primer pair: AA383349_seg32F (SEQ ID NO: 359) forward primer; and AA383349_seg32R (SEQ ID NO: 360) reverse primer.

The present invention also preferably encompasses any amplicon obtained through the use of any suitable primer pair; for example, for the above experiment, the following amplicon was obtained as a non-limiting illustrative example only of a suitable amplicon: AA383349_seg32 (SEQ ID NO: 361).

### Description for Cluster AA583399

Cluster AA583399 features 16 transcript(s) and 20 segment(s) of interest, the names for which are given in Tables 22 and 23, respectively, the sequences themselves are given in the “Sequence Listing” of the application. The selected protein variants are given in table 24.

TABLE 22
Transcripts of interest
Transcript Name

AA583399_PEA_1_T0 (SEQ ID NO: 37)
AA583399_PEA_1_T1 (SEQ ID NO: 38)
AA583399_PEA_1_T2 (SEQ ID NO: 39)
AA583399_PEA_1_T3 (SEQ ID NO: 40)
AA583399_PEA_1_T4 (SEQ ID NO: 41)
AA583399_PEA_1_T5 (SEQ ID NO: 42)
AA583399_PEA_1_T6 (SEQ ID NO: 43)
AA583399_PEA_1_T7 (SEQ ID NO: 44)
AA583399_PEA_1_T8 (SEQ ID NO: 45)
AA583399_PEA_1_T9 (SEQ ID NO: 46)
AA583399_PEA_1_T10 (SEQ ID NO: 47)
AA583399_PEA_1_T11 (SEQ ID NO: 48)
AA583399_PEA_1_T12 (SEQ ID NO: 49)
AA583399_PEA_1_T15 (SEQ ID NO: 50)
AA583399_PEA_1_T16 (SEQ ID NO: 51)
AA583399_PEA_1_T17 (SEQ ID NO: 52)

TABLE 23
Segments of interest
Segment Name

AA583399_PEA_1_node_0 (SEQ ID NO: 53)
AA583399_PEA_1_node_3 (SEQ ID NO: 54)
AA583399_PEA_1_node_9 (SEQ ID NO: 55)
AA583399_PEA_1_node_10 (SEQ ID NO: 56)
AA583399_PEA_1_node_12 (SEQ ID NO: 57)
AA583399_PEA_1_node_14 (SEQ ID NO: 58)
AA583399_PEA_1_node_21 (SEQ ID NO: 59)
AA583399_PEA_1_node_24 (SEQ ID NO: 60)
AA583399_PEA_1_node_25 (SEQ ID NO: 61)
AA583399_PEA_1_node_29 (SEQ ID NO: 62)
AA583399_PEA_1_node_1 (SEQ ID NO: 63)
AA583399_PEA_1_node_2 (SEQ ID NO: 64)
AA583399_PEA_1_node_4 (SEQ ID NO: 65)
AA583399_PEA_1_node_5 (SEQ ID NO: 66)
AA583399_PEA_1_node_6 (SEQ ID NO: 67)
AA583399_PEA_1_node_7 (SEQ ID NO: 68)
AA583399_PEA_1_node_8 (SEQ ID NO: 69)
AA583399_PEA_1_node_11 (SEQ ID NO: 70)
AA583399_PEA_1_node_19 (SEQ ID NO: 71)
AA583399_PEA_1_node_27 (SEQ ID NO: 72)

TABLE 24
Proteins of interest

| Protein Name | Corresponding Transcript(s) |
|---|---|
| AA583399_PEA_1_P3 (SEQ ID NO: 77) | AA583399_PEA_1_T1 (SEQ ID NO: 38); |
| | AA583399_PEA_1_T6 (SEQ ID NO: 43); |
| | AA583399_PEA_1_T9 (SEQ ID NO: 46) |
| AA583399_PEA_1_P2 (SEQ ID NO: 78) | AA583399_PEA_1_T3 (SEQ ID NO: 40); |
| | AA583399_PEA_1_T4 (SEQ ID NO: 41); |
| | AA583399_PEA_1_T5 (SEQ ID NO: 42) |
| AA583399_PEA_1_P4 (SEQ ID NO: 79) | AA583399_PEA_1_T7 (SEQ ID NO: 44) |
| AA583399_PEA_1_P5 (SEQ ID NO: 80) | AA583399_PEA_1_T8 (SEQ ID NO: 45) |
| AA583399_PEA_1_P6 (SEQ ID NO: 81) | AA583399_PEA_1_T12 (SEQ ID NO: 49); |
| | AA583399_PEA_1_T16 (SEQ ID NO: 51) |
| AA583399_PEA_1_P8 (SEQ ID NO: 82) | AA583399_PEA_1_T17 (SEQ ID NO: 52) |
| AA583399_PEA_1_P10 (SEQ ID NO: 83) | AA583399_PEA_1_T0 (SEQ ID NO: 37) |
| AA583399_PEA_1_P11 (SEQ ID NO: 84) | AA583399_PEA_1_T2 (SEQ ID NO: 39) |
| AA583399_PEA_1_P12 (SEQ ID NO: 85) | AA583399_PEA_1_T10 (SEQ ID NO: 47); |
| | AA583399_PEA_1_T11 (SEQ ID NO: 48) |
| AA583399_PEA_1_P14 (SEQ ID NO: 86) | AA583399_PEA_1_T15 (SEQ ID NO: 50) |

These sequences are variants of the known protein Myeloma overexpressed gene protein (SEQ ID NO: 73) (SwissProt accession identifier MYEO_HUMAN, known also according to the synonyms Oncogene in multiple myeloma), referred to herein as the previously known protein.

Known polymorphisms for this sequence are as shown in Table 25.

TABLE 25
Amino acid mutations for Known Protein

| SNP position(s) | |
|---|---|
| on amino | |
| acid sequence | Comment |
| 159 | A -> V (in dbSNP: 7103126). /FTId = VAR_016603. |
| 198 | R -> Q |
| 219 | V -> M |
| 271 | G -> R |

WO20051B0000928 and U.S. patent application Ser. No. 11/050,875, filed on Jan. 27, 2005 and assigned to the applicant of the present invention, disclose polynucleotides and their respective encoded polypeptides of AA583399 contig, and assays and methods of use thereof in the diagnosis of colon cancer. In these applications the AA583399 sequences are described as being overexpressed in brain malignant tumors, epithelial malignant tumors, normal and cancerous colon tissues.

Unexpectedly, AA583399 variants and known wild type polynucleotides and their respective encoded polypeptides are now disclosed to be differentially expressed in ovarian cancer and lung cancer, and therefore may be contemplated within the scope of the present invention as diagnostic markers for these diseases.

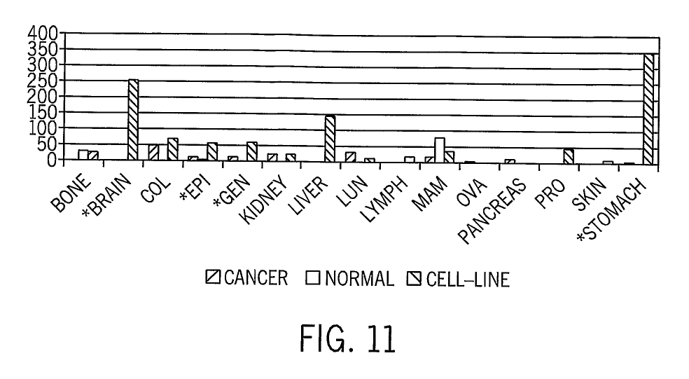

Cluster AA583399 can be used as a diagnostic marker according to overexpression of transcripts of this cluster in cancer. Expression of such transcripts in normal tissues is also given according to the previously described methods. The term “number” in the left hand column of the table and the numbers on the y-axis of the FIG. 11 refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 11 and Table 26. This cluster is overexpressed (at least at a minimum level) in the following pathological conditions: brain malignant tumors, epithelial malignant tumors, a mixture of malignant tumors from different tissues and gastric carcinoma.

TABLE 26
Normal tissue distribution

| Name of Tissue | Number |
|---|---|
| bone | 32 |
| brain | 0 |
| colon | 0 |
| epithelial | 4 |
| general | 2 |
| kidney | 0 |
| liver | 0 |
| lung | 2 |
| lymph nodes | 0 |
| breast | 79 |
| ovary | 0 |
| pancreas | 0 |
| prostate | 0 |
| skin | 0 |
| stomach | 0 |

TABLE 27
P values and ratios for expression in cancerous tissue

| Name of Tissue | P1 | P2 | SP1 | R3 | SP2 | R4 |
|---|---|---|---|---|---|---|
| bone | 9.2e−01 | 5.8e−01 | 1 | 0.5 | 9.1e−01 | 0.8 |
| brain | 9.8e−01 | 1.4e−01 | 1 | 0.8 | 2.0e−09 | 11.1 |
| colon | 1.6e−01 | 7.3e−02 | 3.4e−01 | 2.5 | 2.7e−01 | 2.7 |
| epithelial | 3.0e−03 | 1.3e−04 | 1.1e−01 | 2.3 | 5.4e−06 | 4.1 |
| general | 1.1e−05 | 3.2e−10 | 2.0e−03 | 4.5 | 1.1e−17 | 10.7 |
| kidney | 6.5e−01 | 5.1e−01 | 1 | 1.1 | 7.0e−01 | 1.5 |
| liver | 1 | 1.3e−01 | 1 | 1.0 | 1.6e−01 | 2.2 |
| lung | 1.7e−01 | 2.7e−01 | 1.7e−01 | 3.5 | 2.4e−01 | 2.5 |
| lymph nodes | 1 | 5.7e−01 | 1 | 1.0 | 5.8e−01 | 1.7 |
| breast | 8.8e−01 | 8.6e−01 | 9.0e−01 | 0.5 | 8.5e−01 | 0.6 |
| ovary | 1.6e−01 | 1.9e−01 | 1 | 1.1 | 1 | 1.1 |
| pancreas | 3.3e−01 | 4.4e−01 | 4.2e−01 | 2.4 | 5.3e−01 | 1.9 |
| prostate | 1 | 7.8e−01 | 1 | 1.0 | 5.6e−01 | 1.7 |
| skin | 1 | 4.4e−01 | 1 | 1.0 | 6.4e−01 | 1.6 |
| stomach | 3.6e−01 | 1.3e−01 | 1 | 1.1 | 1.8e−03 | 3.8 |

As noted above, cluster AA583399 features 16 transcript(s), which were listed in Table 22 above. These transcript(s) encode for protein(s) which are variant(s) of protein Myeloma overexpressed gene protein (SEQ ID NO: 73). A description of each variant protein according to the present invention is now provided.

Variant protein AA583399_PEA_{—}1_P3 (SEQ ID NO: 77) according to the present invention has an amino acid sequence as given in the “Sequence Listing” of the application; it is encoded by transcript(s) AA583399_PEA_{—}1_T1 (SEQ ID NO: 38). The location of the variant protein was determined according to results from a number of different software programs and analyses, including analyses from SignalP and other specialized programs. The variant protein is believed to be secreted. The protein localization is believed to be secreted because both signal-peptide prediction programs predict that this protein has a signal peptide.

Variant protein AA583399_PEA_{—}1_P3 (SEQ ID NO: 77) also has the following non-silent SNPs (Single Nucleotide Polymorphisms) as listed in Table 28, (given according to their position(s) on the amino acid sequence, with the alternative amino acid(s) listed).

TABLE 28
Amino acid mutations

| SNP position(s) on | Alternative |
|---|---|
| amino acid sequence | amino acid(s) |
| 159 | V -> A |
| 198 | R -> Q |
| 219 | M -> V |
| 271 | G -> R |
| 284 | P -> T |

Variant protein AA583399_PEA_{—}1P3 (SEQ ID NO: 77) is encoded by the following transcript(s): AA583399_PEA_{—}1_T1 (SEQ ID NO: 38), for which the coding portion starts at position 587 and ends at position 1525. The transcript also has the following SNPs as listed in Table 29 (given according to their position on the nucleotide sequence, with the alternative nucleic acid listed).

TABLE 29
Nucleic acid SNPs

| | SNP position on nucleotide |
|---|---|
| Polymorphism | sequence |
| A -> G | 380, 1241 |
| C -> T | 805, 1653 |
| T -> C | 1062 |
| G -> A | 1179 |
| G -> C | 1397 |
| C -> A | 1436 |
| T -> G | 1657 |

Variant protein AA583399_PEA_{—}1_P2 (SEQ ID NO: 78) according to the present invention has an amino acid sequence as given in the “Sequence Listing” of the application; it is encoded by transcript(s) AA583399_PEA_{—}1_T3 (SEQ ID NO: 40). An alignment is given to the known protein (Myeloma overexpressed gene protein, SEQ ID NOs:73-76) in the alignment table, on the attached CDROM. One or more alignments to one or more previously published protein sequences are given in the alignment table, on the attached CDROM. A brief description of the relationship of the variant protein according to the present invention to each such aligned protein is as follows:

Comparison report between AA583399_PEA_{—}1_P2 (SEQ ID NO: 78) and MYEO_HUMAN_V1 (SEQ ID NO: 74):

1. An isolated chimeric polypeptide encoding for AA583399_PEA_{—}1_P2 (SEQ ID NO: 78), comprising a first amino acid sequence being at least about 90% homologous to MFTRQAGHFVEGSKAGRSRGRLCLSQALRVAVRGAFVSLWFAAGAGDRERNKGDKGAQTGAGLSQEAE DVDVSRARRVTDAPQGTLCGTGNRNSGSQSARVVGVAHLGEAFRVGVEQAISSCPEEVHGRHGLSMEIM WARMDVALRSPGRGLLAGAGALCMTLAESSCPDYERGRRACLTLHRHPTPHCSTWGLPLRVAGSWLTV VTVEALGGWRMGVRRTGQVGPTMHPPVSGASPLLLHHLLLLIIILTC corresponding to amino acids 59-313 of MYEO_HUMAN_V1 (SEQ ID NO: 74), which also corresponds to amino acids 1-255 of AA583399_PEA_{—}1_P2 (SEQ ID NO: 78).

It should be noted that the known protein sequence (MYEO_HUMAN (SEQ ID NO:431)) has one or more changes than the sequence given in SEQ ID NO:74, and named as being the amino acid sequence for MYEO_HUMAN_V1 (SEQ ID NO: 74). These changes were previously known to occur and are listed in the table below.

TABLE 30
Changes to MYEO_HUMAN_V1 (SEQ ID NO: 74)

| SNP position(s) on amino | |
|---|---|
| acid sequence | Type of change |
| 160 | variant |
| 220 | conflict |

The location of the variant protein was determined according to results from a number of different software programs and analyses, including analyses from SignalP and other specialized programs. The variant protein is believed to be secreted. The protein localization is believed to be secreted because one of the two signal-peptide prediction programs (HMM:Signal peptide,NN:NO) predicts that this protein has a signal peptide.

Variant protein AA583399_PEA_{—}1_P2 (SEQ ID NO: 78) is encoded by the following transcript(s): AA583399_PEA_{—}1_T3 (SEQ ID NO: 40), for which the coding portion starts at position 689 and ends at position 1453. The transcript also has the following SNPs as listed in Table 31 (given according to their position on the nucleotide sequence, with the alternative nucleic acid listed).

TABLE 31
Nucleic acid SNPs

| | SNP position on nucleotide |
|---|---|
| Polymorphism | sequence |
| A -> G | 380, 1169 |
| C -> T | 733, 1581 |
| T -> C | 990 |
| G -> A | 1107 |
| G -> C | 1325 |
| C -> A | 1364 |
| T -> G | 1585 |

Variant protein AA583399_PEA_{—}1_P4 (SEQ ID NO: 79) according to the present invention has an amino acid sequence as given in the “Sequence Listing” of the application; it is encoded by transcript(s) AA583399_PEA_{—}1_T7 (SEQ ID NO: 44). An alignment is given to the known protein (Myeloma overexpressed gene protein, SEQ ID NOs:73-76) in the alignment table, on the attached CDROM. One or more alignments to one or more previously published protein sequences are given in the alignment table, on the attached CDROM. A brief description of the relationship of the variant protein according to the present invention to each such aligned protein is as follows:

Comparison report between AA583399_PEA_{—}1_P4 (SEQ ID NO: 79) and MYEO_HUMAN_V1 (SEQ ID NO: 74):

1. An isolated chimeric polypeptide encoding for AA583399_PEA_{—}1_P4 (SEQ ID NO: 79), comprising a first amino acid sequence being at least about 70%, optionally at least 80%, preferably at least 85%, more preferably at least 90% and most preferably at least 95% homologous to a polypeptide having the sequence MSDLFIGFLVCSLSPLGTGTRCSCSPG (SEQ ID NO: 531) corresponding to amino acids 1-27 of AA583399_PEA_{—}1_P4 (SEQ ID NO: 79), and a second amino acid sequence being at least about 90% homologous to RNSGSQSARVVGVAHLGEAFRVGVEQAISSCPEEVHGRHGLSMEIMWARMDVALRSPGRGLLAG AGALCMTLAESSCPDYERGRRACLTLHRHPTPHCSTWGLPLRVAGSWLTVVTVEALGGWRMGVRRTGQ VGPTMHPPPVSGASPLLLHHLLLLLLIIILTC corresponding to amino acids 150-313 of MYEO_HUMAN_V1 (SEQ ID NO: 74), which also corresponds to amino acids 28-191 of AA583399_PEA 11P4 (SEQ ID NO: 79), wherein said first amino acid sequence and second amino acid sequence are contiguous and in a sequential order.

2. An isolated polypeptide encoding for a head of AA583399_PEA_{—}1_P4 (SEQ ID NO: 79), comprising a polypeptide being at least 70%, optionally at least about 80%, preferably at least about 85%, more preferably at least about 90% and most preferably at least about 95% homologous to the sequence

| | |
|---|---|
| MSDLFIGFLVCSLSPLGTGTRCSCSPG | (SEQ ID NO: 531) |
| of | |
| AA583399_PEA_1_P4. | (SEQ ID NO: 79) |

The location of the variant protein was determined according to results from a number of different software programs and analyses, including analyses from SignalP and other specialized programs. The variant protein is believed to be secreted. The protein localization is believed to be secreted because both signal-peptide prediction programs predict that this protein has a signal peptide.

Variant protein AA583399_PEA_{—}1_P4 (SEQ ID NO: 79) also has the following non-silent SNPs (Single Nucleotide Polymorphisms) as listed in Table 32, (given according to their position(s) on the amino acid sequence, with the alternative amino acid(s) listed).

TABLE 32
Amino acid mutations

| SNP position(s) on | Alternative |
|---|---|
| amino acid sequence | amino acid(s) |
| 37 | V -> A |
| 76 | R -> Q |
| 97 | M -> V |
| 149 | G -> R |
| 162 | P -> T |

Variant protein AA583399_PEA_{—}1_P4 (SEQ ID NO: 79) is encoded by the following transcript(s): AA583399_PEA_{—}1_T7 (SEQ ID NO: 44), for which coding portion starts at position 789 and ends at position 1361. The transcript also has the following SNPs as listed in Table 33 (given according to their position on the nucleotide sequence, with the alternative nucleic acid listed).

TABLE 33
Nucleic acid SNPs

| | SNP position on nucleotide |
|---|---|
| Polymorphism | sequence |
| A -> G | 380, 1077 |
| T -> C | 898 |
| G -> A | 1015 |
| G -> C | 1233 |
| C -> A | 1272 |
| C -> T | 1489 |
| T -> G | 1493 |

Variant protein AA583399_PEA_{—}1_P5 (SEQ ID NO: 80) according to the present invention has an amino acid sequence as given in the “Sequence Listing” of the application; it is encoded by transcript(s) AA583399_PEA_{—}1_T8 (SEQ ID NO: 45). An alignment is given to the known protein (Myeloma overexpressed gene protein SEQ ID NOs:73-76) in the alignment table, on the attached CDROM. One or more alignments to one or more previously published protein sequences are given in the alignment table, on the attached CDROM. A brief description of the relationship of the variant protein according to the present invention to each such aligned protein is as follows:

Comparison report between AA583399_PEA_{—}1_P5 (SEQ ID NO: 80) and MYEO_HUMAN_V₂ (SEQ ID NO: 75):

1. An isolated chimeric polypeptide encoding for AA583399_PEA_{—}1_P5 (SEQ ID NO: 80), comprising a first amino acid sequence being at least about 90% homologous to MEIMWARMDVALRSPGRGLLAGAGALCMTLAESSCPDYERGRRACLTLHRBPTPHCSTWGLPLRVAGS WLTVVTVEALGGWRMGVRRTGQVGPTMHPPPVSGASPLLZLHHLLLLLLIIILLTC corresponding to amino acids 192-313 of MYEO_HUMAN_V2 (SEQ ID NO: 75), which also corresponds to amino acids 1-122 of AA583399_PEA_{—}1_P5 (SEQ ID NO: 80).

It should be noted that the known protein sequence (MYEO_HUMAN (SEQ ID NO:431)) has one or more changes than the sequence given in SEQ ID NO: 75, and named as being the amino acid sequence for MYEO_HUMAN_V2 (SEQ ID NO: 75). These changes were previously known to occur and are listed in the table below.

TABLE 34
Changes to MYEO_HUMAN_V2 (SEQ ID NO: 75)

| SNP position(s) on amino | |
|---|---|
| acid sequence | Type of change |
| 220 | conflict |

The location of the variant protein was determined according to results from a number of different software programs and analyses, including analyses from SignalP and other specialized programs. The variant protein is believed to be secreted. The protein localization is believed to be secreted because one of the two signal-peptide prediction programs (HMM:Signal peptide,NN:NO) predicts that this protein has a signal peptide.

Variant protein AA583399_PEA_{—}1_P5 (SEQ ID NO: 80) also has the following non-silent SNPs (Single Nucleotide Polymorphisms) as listed in Table 35, (given according to their position(s) on the amino acid sequence, with the alternative amino acid(s) listed).

TABLE 35
Amino acid mutations

| SNP position(s) on amino acid | |
|---|---|
| sequence | Alternative amino acid(s) |
| 7 | R -> Q |
| 28 | M -> V |
| 80 | G -> R |
| 93 | P -> T |

Variant protein AA583399_PEA_{—}1_P5 (SEQ ID NO: 80) is encoded by the following transcript(s): AA583399_PEA_{—}1_T8 (SEQ ID NO: 45), for which the coding portion starts at position 849 and ends at position 1214. The transcript also has the following SNPs as listed in Table 36 (given according to their position on the nucleotide sequence, with the alternative nucleic acid listed; AA583399_PEA_{—}1_P5 (SEQ ID NO: 80)).

TABLE 36
Nucleic acid SNPs

| | SNP position on nucleotide |
|---|---|
| Polymorphism | sequence |
| A -> G | 380, 930 |
| C -> T | 494, 1342 |
| T -> C | 751 |
| G -> A | 868 |
| G -> C | 1086 |
| C -> A | 1125 |
| T -> G | 1346 |

Variant protein AA583399_PEA_{—}1_P6 (SEQ ID NO: 81) according to the present invention has an amino acid sequence as given in the “Sequence Listing” of the application; it is encoded by transcript(s) AA583399_PEA_{—}1_T12 (SEQ ID NO: 49). The location of the variant protein was determined according to results from a number of different software programs and analyses, including analyses from SignalP and other specialized programs. The variant protein is believed to be located intracellularly with regard to the cell. The protein localization is believed to be intracellularly because neither of the trans-membrane region prediction programs predicted a trans-membrane region for this protein. In addition both signal-peptide prediction programs predict that this protein is a non-secreted protein.

Variant protein AA583399_PEA_{—}1_P6 (SEQ ID NO: 81) is encoded by the following transcript(s): AA583399_PEA_{—}1_T12 (SEQ ID NO: 49), for which the coding portion starts at position 39 and ends at position 371. The transcript also has the following SNPs as listed in Table 37 (given according to their position on the nucleotide sequence, with the alternative nucleic acid listed).

TABLE 37
Nucleic acid SNPs

| | SNP position on nucleotide |
|---|---|
| Polymorphism | sequence |
| A -> G | 198, 1145 |
| C -> A | 538 |
| C -> G | 659 |
| C -> T | 1009 |

Variant protein AA583399_PEA_{—}1_P8 (SEQ ID NO: 82) according to the present invention has an amino acid sequence as given in the “Sequence Listing” of the application; it is encoded by transcript(s) AA583399_PEA_{—}1_T17 (SEQ ID NO: 52). The location of the variant protein was determined according to results from a number of different software programs and analyses, including analyses from SignalP and other specialized programs. The variant protein is believed to be located intracellularly with regard to the cell. The protein localization is believed to be intracellularly because neither of the trans-membrane region prediction programs predicted a trans-membrane region for this protein. In addition both signal-peptide prediction programs predict that this protein is a non-secreted protein.

Variant protein AA583399_PEA_{—}1_P8 (SEQ ID NO: 82) is encoded by the following transcript(s): AA583399_PEA_{—}1_T17 (SEQ ID NO: 52), for which the coding portion starts at position 191 and ends at position 400.

Variant protein AA583399_PEA_{—}1_P10 (SEQ ID NO: 83) according to the present invention has an amino acid sequence as given in the “Sequence Listing” of the application; it is encoded by transcript(s) AA583399 PEA_{—}1_T0 (SEQ ID NO: 37). An alignment is given to the known protein (Myeloma overexpressed gene protein SEQ ID NOs:73-76) in the alignment table, on the attached CDROM. One or more alignments to one or more previously published protein sequences are given in the alignment table, on the attached CDROM. A brief description of the relationship of the variant protein according to the present invention to each such aligned protein is as follows:

Comparison report between AA583399_PEA_{—}1_P10 (SEQ ID NO: 83) and MYEO_HUMAN_V3 (SEQ ID NO: 76):

1. An isolated chimeric polypeptide encoding for AA583399_PEA_{—}1P10 (SEQ ID NO: 83), comprising a first amino acid sequence being at least about 90% homologous to MFTRQAGHFVEGSKAGRSRGRLCLSQALRVAVRGAFVSLWFAAGAGDRERNKGDKGAQTGAGLSQEAE DVDVSRARRVTDAPQGTLCGTGNRNSGSQSARAVGVAHLGEAFRVGVEQAISSCPEEVHGRHGLSMEIM WAQMDVALRSPGRGLLAGAGALCMTLAESSCPDYERGRRACLTLHRHPTPHCSTWGLPLRVAGSWLTV VTVEALGRWRMGVRRTGQVGPTMHPPVSGASPLLLHHLLLLLLIIILTC corresponding to amino acids 59-313 of MYEO_HUMAN_V3 (SEQ ID NO: 76), which also corresponds to amino acids 1-255 of AA583399_PEA_{—}1_P10 (SEQ ID NO: 83).

It should be noted that the known protein sequence (MYEO_HUMAN (SEQ ID NO:431)) has one or more changes than the sequence given in SEQ ID NO:76, and named as being the amino acid sequence for MYEO_HUMAN_V3 (SEQ ID NO: 76). These changes were previously known to occur and are listed in the table below.

TABLE 38
Changes to MYEO_HUMAN_V3 (SEQ ID NO: 76)

| SNP position(s) on amino | |
|---|---|
| acid sequence | Type of change |
| 199 | conflict |
| 220 | conflict |
| 272 | conflict |

The location of the variant protein was determined according to results from a number of different software programs and analyses, including analyses from SignalP and other specialized programs. The variant protein is believed to be secreted. The protein localization is believed to be secreted because one of the two signal-peptide prediction programs (HMM:Signal peptide,NN:NO) predicts that this protein has a signal peptide.

Variant protein AA583399_PEA_{—}1_P10 (SEQ ID NO: 83) also has the following non-silent SNPs (Single Nucleotide Polymorphisms) as listed in Table 39, (given according to their position(s) on the amino acid sequence, with the alternative amino acid(s) listed).

TABLE 39
Amino acid mutations

| SNP position(s) on amino acid | |
|---|---|
| sequence | Alternative amino acid(s) |
| 101 | A -> V |
| 140 | Q -> R |
| 161 | M -> V |
| 213 | R -> G |
| 226 | P -> T |

Variant protein AA583399_PEA_{—}1_P10 (SEQ ID NO: 83) is encoded by the following transcript(s): AA583399_PEA_{—}1_T0 (SEQ ID NO: 37), for which the coding portion starts at position 857 and ends at position 1621. The transcript also has the following SNPs as listed in Table 40 (given according to their position on the nucleotide sequence, with the alternative nucleic acid listed).

TABLE 40
Nucleic acid SNPs

| | SNP position on nucleotide |
|---|---|
| Polymorphism | sequence |
| A -> G | 380, 1337 |
| C -> T | 901, 1749 |
| T -> C | 1158 |
| G -> A | 1275 |
| G -> C | 1493 |
| C -> A | 1532 |
| T -> G | 1753 |

Variant protein AA583399_PEA_{—}1_P1 (SEQ ID NO: 84) according to the present invention has an amino acid sequence as given in the “Sequence Listing” of the application; it is encoded by transcript(s) AA583399_PEA_{—}1_T2 (SEQ ID NO: 39). The location of the variant protein was determined according to results from a number of different software programs and analyses, including analyses from SignalP and other specialized programs. The variant protein is believed to be secreted. The protein localization is believed to be secreted because both signal-peptide prediction programs predict that this protein has a signal peptide.

Variant protein AA583399_PEA_{—}1_P11 (SEQ ID NO: 84) also has the following non-silent SNPs (Single Nucleotide Polymorphisms) as listed in Table 41, (given according to their position(s) on the amino acid sequence, with the alternative amino acid(s) listed).

TABLE 41
Amino acid mutations

| SNP position(s) on amino acid | |
|---|---|
| sequence | Alternative amino acid(s) |
| 159 | A -> V |
| 198 | R -> Q |
| 219 | V -> M |
| 271 | G -> R |
| 284 | P -> T |

Variant protein AA583399_PEA_{—}1_P11 (SEQ ID NO: 84) is encoded by the following transcript(s): AA583399_PEA_{—}1_T2 (SEQ ID NO: 39), for which the coding portion starts at position 493 and ends at position 1431. The transcript also has the following SNPs as listed in Table 42 (given according to their position on the nucleotide sequence, with the alternative nucleic acid listed).

TABLE 42
Nucleic acid SNPs

| | SNP position on nucleotide |
|---|---|
| Polymorphism | sequence |
| A -> G | 380, 1147 |
| C -> T | 711, 1559 |
| T -> C | 968 |
| G -> A | 1085 |
| G -> C | 1303 |
| C -> A | 1342 |
| T -> G | 1563 |

Variant protein AA583399_PEA_{—}1_P12 (SEQ ID NO: 85) according to the present invention has an amino acid sequence as given in the “Sequence Listing” of the application; it is encoded by transcript(s) AA583399_PEA_{—}1_T10 (SEQ ID NO: 47) and AA583399_PEA_{—}1_T11 (SEQ ID NO: 48). The location of the variant protein was determined according to results from a number of different software programs and analyses, including analyses from SignalP and other specialized programs. The variant protein is believed to be located intracellularly with regard to the cell. The protein localization is believed to be intracellularly because neither of the trans-membrane region prediction programs predicted a trans-membrane region for this protein. In addition both signal-peptide prediction programs predict that this protein is a non-secreted protein.

Variant protein AA583399_PEA_{—}1_P12 (SEQ ID NO: 85) is encoded by the following transcript(s): AA583399_PEA_{—}1_T10 (SEQ ID NO: 47) and AA583399_PEA_{—}1_T11 (SEQ ID NO: 48), for which the coding portion starts at position 191 and ends at position 367. The transcript also has the following SNPs as listed in Table 43 (given according to their position on the nucleotide sequence, with the alternative nucleic acid listed).

TABLE 43
Nucleic acid SNPs

| | SNP position on nucleotide |
|---|---|
| Polymorphism | sequence |
| A -> G | 380 |

The coding portion of transcript AA583399_PEA_{—}1_T11 (SEQ ID NO: 48) starts at position 191 and ends at position 367. The transcript also has the following SNPs as listed in Table 44 (given according to their position on the nucleotide sequence, with the alternative nucleic acid listed).

TABLE 44
Nucleic acid SNPs

| | SNP position on nucleotide |
|---|---|
| Polymorphism | sequence |
| A -> G | 380 |

Variant protein AA583399_PEA_{—}1_P14 (SEQ ID NO: 86) according to the present invention has an amino acid sequence as given in the “Sequence Listing” of the application; it is encoded by transcript(s) AA583399_PEA_{—}1_T15 (SEQ ID NO: 50). The location of the variant protein was determined according to results from a number of different software programs and analyses, including analyses from SignalP and other specialized programs. The variant protein is believed to be located intracellularly with regard to the cell. The protein localization is believed to be intracellularly because neither of the trans-membrane region prediction programs predicted a trans-membrane region for this protein. In addition both signal-peptide prediction programs predict that this protein is a non-secreted protein.

Variant protein AA583399_PEA_{—}1_P14 (SEQ ID NO: 86) is encoded by the following transcript(s): AA583399_PEA_{—}1_T15 (SEQ ID NO: 50), for which the coding portion starts at position 43 and ends at position 210.

As noted above, cluster AA583399 features 20 segment(s), which were listed in Table 23 above and for which the sequence(s) are given in the “Sequence Listing” of the application. These segment(s) are portions of nucleic acid sequence(s) which are described herein separately because they are of particular interest. A description of some of the segments according to the present invention is now provided.

Segment cluster AA583399_PEA_{—}1_node_{—}0 (SEQ ID NO: 53) according to the present invention is supported by 24 libraries. The number of libraries was determined as previously described. This segment can be found in the following transcript(s): AA583399_PEA_{—}1_T0 (SEQ ID NO: 37), AA583399_PEA_{—}1_T1 (SEQ ID NO: 38), AA583399_PEA 11T2 (SEQ ID NO: 39), AA583399_PEA_{—}1_T3 (SEQ ID NO: 40), AA583399_PEA_{—}1_T4 (SEQ ID NO: 41), AA583399_PEA_{—}11T5 (SEQ ID NO: 42), AA583399_PEA_{—}1_T6 (SEQ ID NO: 43), AA583399_PEA_{—}1_T7 (SEQ ID NO: 44), AA583399_PEA 11T8 (SEQ ID NO: 45), AA583399_PEA_{—}1T9 (SEQ ID NO: 46), AA583399_PEA_{—}1_T10 (SEQ ID NO: 47), AA583399_PEA_{—}1_T1 (SEQ ID NO: 48) and AA583399_PEA_{—}1-T17 (SEQ ID NO: 52). Table 45 below describes the starting and ending position of this segment on each transcript.

TABLE 45
Segment location on transcripts

| | Segment | Segment |
|---|---|---|
| | starting | ending |
| Transcript name | position | position |
| AA583399_PEA_1_T0 (SEQ ID NO: 37) | 1 | 355 |
| AA583399_PEA_1_T1 (SEQ ID NO: 38) | 1 | 355 |
| AA583399_PEA_1_T2 (SEQ ID NO: 39) | 1 | 355 |
| AA583399_PEA_1_T3 (SEQ ID NO: 40) | 1 | 355 |
| AA583399_PEA_1_T4 (SEQ ID NO: 41) | 1 | 355 |
| AA583399_PEA_1_T5 (SEQ ID NO: 42) | 1 | 355 |
| AA583399_PEA_1_T6 (SEQ ID NO: 43) | 1 | 355 |
| AA583399_PEA_1_T7 (SEQ ID NO: 44) | 1 | 355 |
| AA583399_PEA_1_T8 (SEQ ID NO: 45) | 1 | 355 |
| AA583399_PEA_1_T9 (SEQ ID NO: 46) | 1 | 355 |
| AA583399_PEA_1_T10 (SEQ ID NO: 47) | 1 | 355 |
| AA583399_PEA_1_T11 (SEQ ID NO: 48) | 1 | 355 |
| AA583399_PEA_1_T17 (SEQ ID NO: 52) | 1 | 355 |

Segment cluster AA583399_PEA_{—}1_node_{—}3 (SEQ ID NO: 54) according to the present invention is supported by 4 libraries. The number of libraries was determined as previously described. This segment can be found in the following transcript(s): AA583399_PEA_{—}1_T4 (SEQ ID NO: 41). Table 46 below describes the starting and ending position of this segment on each transcript.

TABLE 46
Segment location on transcripts

| | Segment | |
|---|---|---|
| | starting | Segment |
| Transcript name | position | ending position |
| AA583399_PEA_1_T4 (SEQ ID NO: 41) | 465 | 1120 |

Segment cluster AA583399_PEA_{—}1_node_{—}9 (SEQ ID NO: 55) according to the present invention is supported by 23 libraries. The number of libraries was determined as previously described. This segment can be found in the following transcript(s): AA583399_PEA_{—}1T0 (SEQ ID NO: 37), AA583399_PEA_{—}1_T1 (SEQ ID NO: 38), AA583399_PEA_{—}1_T2 (SEQ ID NO: 39), AA583399_PEA_{—}1_T3 (SEQ ID NO: 40), AA583399_PEA_{—}1_T4 (SEQ ID NO: 41), AA583399_PEA_{—}1_T5 (SEQ ID NO: 42), AA583399_PEA_{—}1_T6 (SEQ ID NO: 43), AA583399_PEA_{—}1T8 (SEQ ID NO: 45) and AA583399_PEA_{—}1_T9 (SEQ ID NO: 46). Table 47 below describes the starting and ending position of this segment on each transcript.

TABLE 47
Segment location on transcripts

| | Segment | |
|---|---|---|
| | starting | Segment |
| Transcript name | position | ending position |
| AA583399_PEA_1_T0 (SEQ ID NO: 37) | 872 | 1131 |
| AA583399_PEA_1_T1 (SEQ ID NO: 38) | 776 | 1035 |
| AA583399_PEA_1_T2 (SEQ ID NO: 39) | 682 | 941 |
| AA583399_PEA_1_T3 (SEQ ID NO: 40) | 704 | 963 |
| AA583399_PEA_1_T4 (SEQ ID NO: 41) | 1528 | 1787 |
| AA583399_PEA_1_T5 (SEQ ID NO: 42) | 778 | 1037 |
| AA583399_PEA_1_T6 (SEQ ID NO: 43) | 776 | 1035 |
| AA583399_PEA_1_T8 (SEQ ID NO: 45) | 465 | 724 |
| AA583399_PEA_1_T9 (SEQ ID NO: 46) | 776 | 1035 |

Segment cluster AA583399_PEA_{—}1_node_{—}10 (SEQ ID NO: 56) according to the present invention is supported by 59 libraries. The number of libraries was determined as previously described. This segment can be found in the following transcript(s): AA583399_PEA_{—}1_T0 (SEQ ID NO: 37), AA583399_PEA_{—}1_T1 (SEQ ID NO: 38), AA583399_PEA_{—}1_T2 (SEQ ID NO: 39), AA583399_PEA_{—}1_T3 (SEQ ID NO: 40), AA583399_PEA_{—}1_T4 (SEQ ID NO: 41), AA583399_PEA_{—}1_T5 (SEQ ID NO: 42), AA583399_PEA_{—}1_T6 (SEQ ID NO: 43), AA583399_PEA_{—}1_T7 (SEQ ID NO: 44), AA583399_PEA_{—}1_T8 (SEQ ID NO: 45) and AA583399_PEA_{—}1_T9 (SEQ ID NO: 46). Table 48 below describes the starting and ending position of this segment on each transcript.

TABLE 48
Segment location on transcripts

| | Segment | Segment |
|---|---|---|
| | starting | ending |
| Transcript name | position | position |
| AA583399_PEA_1_T0 (SEQ ID NO: 37) | 1132 | 2389 |
| AA583399_PEA_1_T1 (SEQ ID NO: 38) | 1036 | 2293 |
| AA583399_PEA_1_T2 (SEQ ID NO: 39) | 942 | 2199 |
| AA583399_PEA_1_T3 (SEQ ID NO: 40) | 964 | 2221 |
| AA583399_PEA_1_T4 (SEQ ID NO: 41) | 1788 | 3045 |
| AA583399_PEA_1_T5 (SEQ ID NO: 42) | 1038 | 2295 |
| AA583399_PEA_1_T6 (SEQ ID NO: 43) | 1036 | 2293 |
| AA583399_PEA_1_T7 (SEQ ID NO: 44) | 872 | 2129 |
| AA583399_PEA_1_T8 (SEQ ID NO: 45) | 725 | 1982 |
| AA583399_PEA_1_T9 (SEQ ID NO: 46) | 1036 | 2293 |

Segment cluster AA583399_PEA_{—}1_node_{—}14 (SEQ ID NO: 58) according to the present invention is supported by 6 libraries. The number of libraries was determined as previously described. This segment can be found in the following transcript(s): AA583399_PEA_{—}1_T12 (SEQ ID NO: 49) and AA583399_PEA_{—}1_T16 (SEQ ID NO: 51). Table 49 below describes the starting and ending position of this segment on each transcript.

TABLE 49
Segment location on transcripts

| | Segment | Segment |
|---|---|---|
| | starting | ending |
| Transcript name | position | position |
| AA583399_PEA_1_T12 (SEQ ID NO: 49) | 1 | 1179 |
| AA583399_PEA_1_T16 (SEQ ID NO: 51) | 1 | 1179 |

Segment cluster AA583399_PEA_{—}1_node_{—}21 (SEQ ID NO: 59) according to the present invention is supported by 15 libraries. The number of libraries was determined as previously described. This segment can be found in the following transcript(s): AA583399_PEA_{—}1_T10 (SEQ ID NO: 47), AA583399_PEA_{—}1_T11 (SEQ ID NO: 48), AA583399_PEA_{—}1_T12 (SEQ ID NO: 49), AA583399_PEA_{—}1_T15 (SEQ ID NO: 50), AA583399_PEA_{—}1_T16 (SEQ ID NO: 51) and AA583399_PEA_{—}1_T17 (SEQ ID NO: 52). Table 50 below describes the starting and ending position of this segment on each transcript.

TABLE 50
Segment location on transcripts

| | Segment | Segment |
|---|---|---|
| | starting | ending |
| Transcript name | position | position |
| AA583399_PEA_1_T10 (SEQ ID NO: 47) | 465 | 633 |
| AA583399_PEA_1_T11 (SEQ ID NO: 48) | 465 | 633 |
| AA583399_PEA_1_T12 (SEQ ID NO: 49) | 1180 | 1348 |
| AA583399_PEA_1_T15 (SEQ ID NO: 50) | 78 | 246 |
| AA583399_PEA_1_T16 (SEQ ID NO: 51) | 1180 | 1348 |
| AA583399_PEA_1_T17 (SEQ ID NO: 52) | 434 | 602 |

Segment cluster AA583399_PEA_{—}1_node_{—}24 (SEQ ID NO: 60) according to the present invention is supported by 7 libraries. The number of libraries was determined as previously described. This segment can be found in the following transcript(s): AA583399_PEA_{—}11T111 (SEQ ID NO: 48), AA583399_PEA_{—}1_T12 (SEQ ID NO: 49), AA583399_PEA_{—}1T15 (SEQ ID NO: 50) and AA583399_PEA_{—}1_T16 (SEQ ID NO: 51). Table 51 below describes the starting and ending position of this segment on each transcript.

TABLE 51
Segment location on transcripts

| | Segment | Segment |
|---|---|---|
| | starting | ending |
| Transcript name | position | position |
| AA583399_PEA_1_T11 (SEQ ID NO: 48) | 634 | 757 |
| AA583399_PEA_1_T12 (SEQ ID NO: 49) | 1349 | 1472 |
| AA583399_PEA_1_T15 (SEQ ID NO: 50) | 247 | 370 |
| AA583399_PEA_1_T16 (SEQ ID NO: 51) | 1349 | 1472 |

Segment cluster AA583399_PEA_{—}1_node_{—}25 (SEQ ID NO: 61) according to the present invention is supported by 1 libraries. The number of libraries was determined as previously described. This segment can be found in the following transcript(s): AA583399_PEA_{—}1_T16 (SEQ ID NO: 51). Table 52 below describes the starting and ending position of this segment on each transcript.

TABLE 52
Segment location on transcripts

| | Segment | Segment |
|---|---|---|
| | starting | ending |
| Transcript name | position | position |
| AA583399_PEA_1_T16 (SEQ ID NO: 51) | 1473 | 1614 |

Segment cluster AA583399_PEA_{—}1_node_{—}29 (SEQ ID NO: 62) according to the present invention is supported by 8 libraries. The number of libraries was determined as previously described. This segment can be found in the following transcript(s): AA583399_PEA_{—}1_T11 (SEQ ID NO: 48), AA583399_PEA_{—}1T12 (SEQ ID NO: 49) and AA583399_PEA_{—}1_T15 (SEQ ID NO: 50). Table 53 below describes the starting and ending position of this segment on each transcript.

TABLE 53
Segment location on transcripts

| | Segment | Segment |
|---|---|---|
| | starting | ending |
| Transcript name | position | position |
| AA583399_PEA_1_T11 (SEQ ID NO: 48) | 832 | 1491 |
| AA583399_PEA_1_T12 (SEQ ID NO: 49) | 1547 | 2206 |
| AA583399_PEA_1_T15 (SEQ ID NO: 50) | 445 | 1104 |

According to an optional embodiment of the present invention, short segments related to the above cluster are also provided. These segments are up to about 120 bp in length, and so are included in a separate description.

Segment cluster AA583399_PEA_{—}1_node_{—}2 (SEQ ID NO: 64) according to the present invention is supported by 24 libraries. The number of libraries was determined as previously described. This segment can be found in the following transcript(s): AA583399_PEA_{—}1_T0 (SEQ ID NO: 37), AA583399_PEA_{—}1_T1 (SEQ ID NO: 38), AA583399_PEA_{—}1_T2 (SEQ ID NO: 39), AA583399_PEA_{—}1_T3 (SEQ ID NO: 40), AA583399_PEA_{—}1_T4 (SEQ ID NO: 41), AA583399_PEA_{—}11T5 (SEQ ID NO: 42), AA583399_PEA_{—}1_T6 (SEQ ID NO: 43), AA583399_PEA_{—}1T7 (SEQ ID NO: 44), AA583399_PEA_{—}1_T8 (SEQ ID NO: 45), A583399_PEA_{—}1T9 (SEQ ID NO: 46), AA583399_PEA_{—}1_T10 (SEQ ID NO: 47), AA583399_PEA_{—}1_T11 (SEQ ID NO: 48) and AA583399_PEA_{—}1_T17 (SEQ ID NO: 52). Table 54 below describes the starting and ending position of this segment on each transcript.

TABLE 54
Segment location on transcripts

| | Segment | Segment |
|---|---|---|
| | starting | ending |
| Transcript name | position | position |
| AA583399_PEA_1_T0 (SEQ ID NO: 37) | 387 | 464 |
| AA583399_PEA_1_T1 (SEQ ID NO: 38) | 387 | 464 |
| AA583399_PEA_1_T2 (SEQ ID NO: 39) | 387 | 464 |
| AA583399_PEA_1_T3 (SEQ ID NO: 40) | 387 | 464 |
| AA583399_PEA_1_T4 (SEQ ID NO: 41) | 387 | 464 |
| AA583399_PEA_1_T5 (SEQ ID NO: 42) | 387 | 464 |
| AA583399_PEA_1_T6 (SEQ ID NO: 43) | 387 | 464 |
| AA583399_PEA_1_T7 (SEQ ID NO: 44) | 387 | 464 |
| AA583399_PEA_1_T8 (SEQ ID NO: 45) | 387 | 464 |
| AA583399_PEA_1_T9 (SEQ ID NO: 46) | 387 | 464 |
| AA583399_PEA_1_T10 (SEQ ID NO: 47) | 387 | 464 |
| AA583399_PEA_1_T11 (SEQ ID NO: 48) | 387 | 464 |
| AA583399_PEA_1_T17 (SEQ ID NO: 52) | 356 | 433 |

Segment cluster AA583399_PEA_{—}1_node_{—}19 (SEQ ID NO: 71) according to the present invention is supported by 1 libraries. The number of libraries was determined as previously described. This segment can be found in the following transcript(s): AA583399_PEA_{—}1_T15 (SEQ ID NO: 50). Table 55 below describes the starting and ending position of this segment on each transcript.

TABLE 55
Segment location on transcripts

| | Segment | Segment |
|---|---|---|
| | starting | ending |
| Transcript name | position | position |
| AA583399_PEA_1_T15 (SEQ ID NO: 50) | 1 | 77 |

Segment cluster AA583399_PEA_{—}1_node_{—}27 (SEQ ID NO: 72) according to the present invention is supported by 4 libraries. The number of libraries was determined as previously described. This segment can be found in the following transcript(s): AA583399_PEA_{—}1_T11 (SEQ ID NO: 48), AA583399_PEA_{—}1_T12 (SEQ ID NO: 49) and AA583399_PEA_{—}1_T15 (SEQ ID NO: 50). Table 56 below describes the starting and ending position of this segment on each transcript.

TABLE 56
Segment location on transcripts

| | Segment | Segment |
|---|---|---|
| | starting | ending |
| Transcript name | position | position |
| AA583399_PEA_1_T11 (SEQ ID NO: 48) | 758 | 831 |
| AA583399_PEA_1_T12 (SEQ ID NO: 49) | 1473 | 1546 |
| AA583399_PEA_1_T15 (SEQ ID NO: 50) | 371 | 444 |

Expression of Homo sapiens myeloma overexpressed gene (in a subset of t(11;14) positive multiple myelomas) (MYEOV) transcripts detectable by or according to seg1-AA583399_seg1 (SEQ ID NO: 364) amplicon and primers AA583399_seg1F (SEQ ID NO: 362) and AA583399_seg1R (SEQ ID NO: 363); or MYEOV transcripts detectable by or according to seg17—AA583399_seg17 (SEQ ID NO: 367) amplicon and primers AA583399_seg17F (SEQ ID NO: 365) and AA583399_seg17R (SEQ ID NO: 366); or MYEOV transcripts detectable by or according to seg30-32—AA583399_seg30-32 (SEQ ID NO: 370) amplicon and primers AA583399_seg30-32F (SEQ ID NO: 368) and AA583399_seg30-32R (SEQ ID NO: 369) was measured by real time PCR.

The sequences of the corresponding primers and amplicons are given below.

The below table 57 gives a conversion between the terms for segments (nodes) in the experimental results and those listed earlier in the description for cluster AA583399:

TABLE 57

| | Segment name within the |
|---|---|
| Segment name for | description for cluster AA583399 |
| AA583399 in experimental results | report |
| seg 1 (WT) (SEQ ID NO: 364) | seg 0 |
| seg30-32 (SEQ ID NO: 370) | seg 27-29 |
| seg 17 (SEQ ID NO: 367) | seg 14 |

Forward Primer (AA583399_seg1F) (SEQ ID NO: 362):
GAATCAGCCCAAAGCCAGG
Reverse Primer (AA583399_seg1R) (SEQ ID NO: 363):
GCTGGTGAAAGCACTGGGTT
Amplicon (AA583399_seg1) (SEQ ID NO: 364):
GAATCAGCCCAAAGCCAGGCGTCCAGGGTCTCCCTCACCTGAAGCTGACT
TTTTCCCCACCTTGGACAGAGGGCGGGAGATGCCATCCCCACTGAACCCA
GTGCTTTCACCAGC
Forward primer (AA583399_seg17F) (SEQ ID NO: 365):
CATTCTCCACGCATCAGATGA
Reverse primer (AA583399_seg17R (SEQ ID NO: 366)):
ACCATCAGATTGGCAGCATG
Amplicon (AA583399 seg17) (SEQ ID NO: 367):
CATTCTCCACGCATCAGATGATCCTGTGGCCCCTCAGTGCCAGGCCCCAC
TGGCCCTCTGCGCACATCAGTGACTCTGATGTTCTCCCCCACCGCATGCT
GCCAATCTGATGGT
Forward Primer (AA583399_seg30-32F)
(SEQ ID NO: 368):
TGGAGATTCCTGGTTTAAAGCATT
Reverse Primer (AA583399_seg30-32R)
(SEQ ID NO: 369):
CCCCAGCTTAGAGCTGCACT
Amplicon (AA583399_seg30-32) (SEQ ID NO: 370):
TGGAGATTCCTGGTTTAAAGCATTTAAAGCCTCTGTGAAAATTTGCCCAG
GCCAACAACTTCACTTTCCACACTCAGTGCCACGAAGTGCAGCTCTAAGC
TGGGG

Expression of Homo sapiens Myeloma Overexpressed Gene (in a Subset of t(11;14) Positive Multiple Myelomas) (MYEOV) AA583399 Transcripts which are Detectable by Amplicon as Depicted in Sequence Name AA583399_Seg1 (SEQ ID NO: 364) in Normal and Cancerous Lung Tissues

Expression of Homo sapiens myeloma overexpressed gene (in a subset of t(11;14) positive multiple myelomas) (MYEOV) transcripts detectable by or according to seg1-AA583399_seg1 (SEQ ID NO: 364) amplicon and primers AA583399_seg1F (SEQ ID NO: 362) and AA583399_seg1R (SEQ ID NO: 363) was measured by real time PCR. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 47, 48, 49, 50, 90, 91, 92, 93, 96, 97, 98 and 99, Table 1_{—}2 above), to obtain a value of fold up-regulation for each sample relative to the median of the normal PM samples.

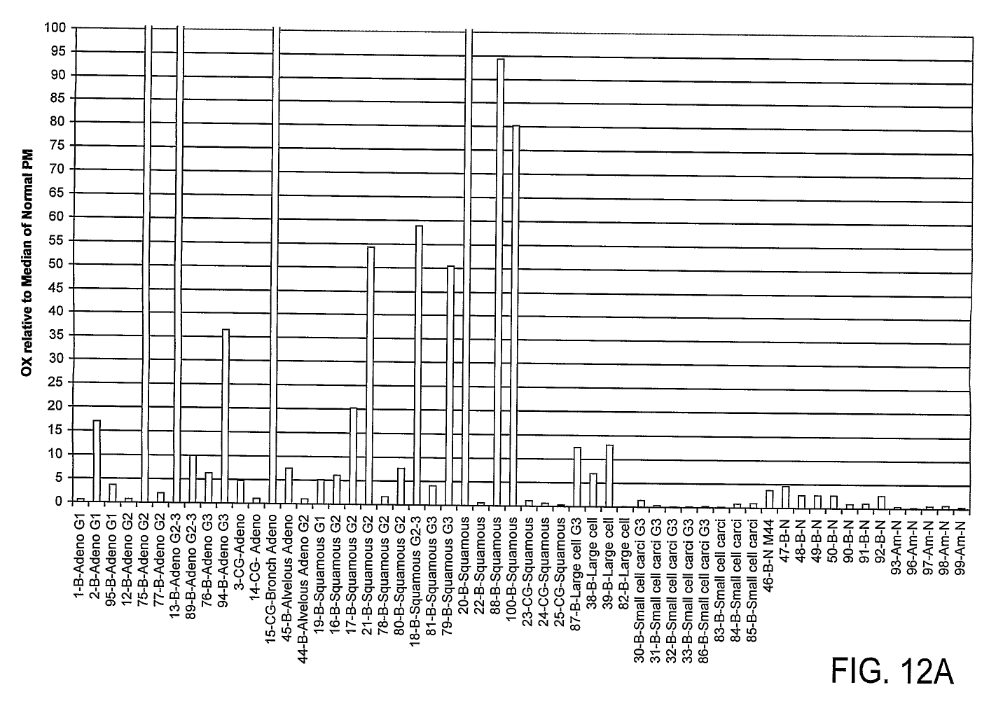

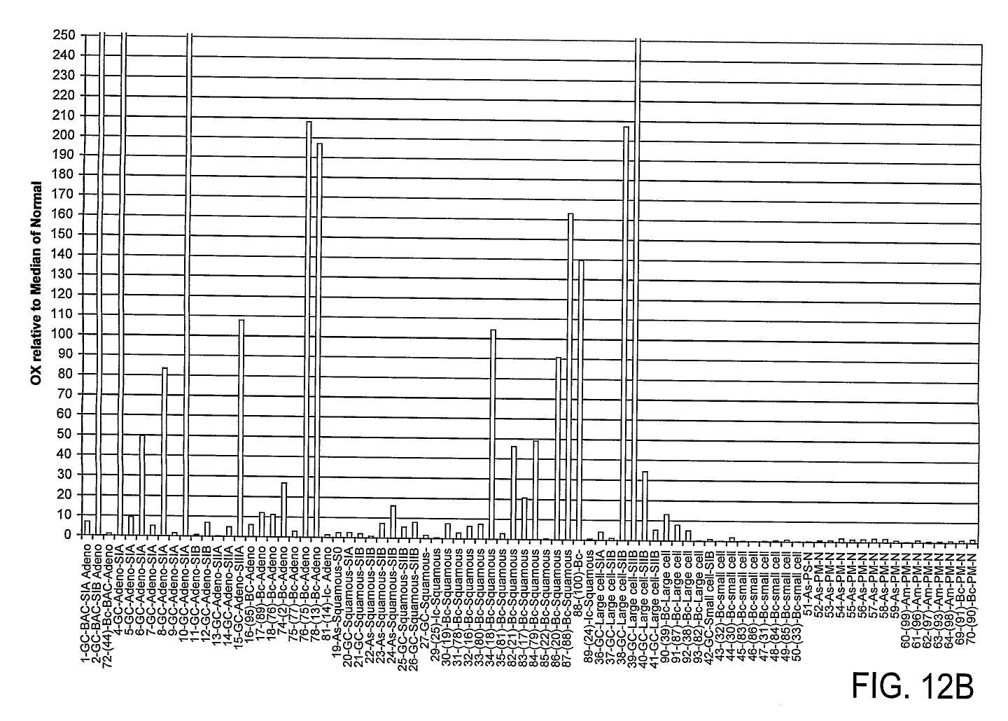

FIG. 12A is a histogram showing over expression of the Homo sapiens myeloma overexpressed gene (in a subset of t(11;14) positive multiple myelomas) (MYEOV) transcripts in cancerous Lung samples relative to the normal samples for transcripts detectable by or according to AA583399_seg1 (SEQ ID NO: 364).

As is evident from FIG. 12A, the expression of Homo sapiens myeloma overexpressed gene (in a subset of t(11;14) positive multiple myelomas) (MYEOV) transcripts detectable by the above amplicon in adenocarcinoma, squamous cell carcinoma, large cell carcinoma samples was significantly higher than in the non-cancerous samples (sample numbers 47, 48, 49, 50, 90, 91, 92, 93, 96, 97, 98 and 99, Table 1_{—}2 above). Notably an over-expression of at least 5 fold was found in 21 out of 35 non-small cell carcinoma samples, specifically in 8 out of 15 adenocarcinoma samples, 10 out of 16 squamous cell carcinoma samples, 3 out of 4 large cell carcinoma samples.

Statistical analysis was applied to verify the significance of these results, as described below.

Threshold of 5 fold over expression was found to differentiate between non-small cell carcinoma and normal samples with P value of 1.85e-004 as checked by exact Fisher test. Threshold of 5 fold over expression was found to differentiate between adenocarcinoma and normal samples with P value of 2.90e-003 as checked by exact Fisher test. Threshold of 5 fold over expression was found to differentiate between squamous cell carcinoma and normal samples with P value of 6.10e-004 as checked by exact Fisher test. Threshold of 5 fold over expression was found to differentiate between large cell carcinoma and normal samples with P value of 7.14e-003 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: AA583399_seg1F (SEQ ID NO: 362) forward primer; and AA583399_seg1R (SEQ ID NO: 363) reverse primer.

The present invention also preferably encompasses any amplicon obtained through the use of any suitable primer pair; for example, for the above experiment, the following amplicon was obtained as a non-limiting illustrative example only of a suitable amplicon: AA583399_seg1 (SEQ ID NO: 364).

Expression of Homo sapiens myeloma overexpressed gene transcripts detectable by or according to seg1—AA583399_seg1WT (SEQ ID NO: 364) amplicon and primers AA583399_seg1WTF (SEQ ID NO: 362) and AA583399_seg1WTR (SEQ ID NO: 363) was further measured by real time PCR using enlarged panel as shown in Table 1_{—}6. In parallel the expression of four housekeeping genes—HPRT1 (GenBank Accession No. NM_{—}000194 (SEQ ID NO:337); amplicon—HPRT1-amplicon (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 51, 52, 53, 54, 55, 56, 57, 58, 59, 60, 61, 62, 63, 64, 69 and 70, Table 1_{—}6 above), to obtain a value of fold up-regulation for each sample relative to the median of the normal samples.

FIG. 12B is a histogram showing over expression of the above-indicated Homo sapiens myeloma overexpressed gene transcripts in cancerous Lung samples relative to the normal samples.

As is evident from FIG. 12B, the expression of Homo sapiens myeloma overexpressed gene transcripts detectable by the above amplicon in non-small cell carcinoma samples—adenocarcinoma, squamous cell carcinoma, large cell carcinoma was significantly higher than in the non-cancerous samples (sample numbers 51, 52, 53, 54, 55, 56, 57, 58, 59, 60, 61, 62, 63, 64, 69 and 70, Table 1_{—}6 above). Notably an over-expression of at least 5 fold was found in 37 out of 57 non-small cell carcinoma samples, specifically in 16 out of 23 adenocarcinoma samples, in 14 out of 24 squamous cell carcinoma samples and in 7 out of 10 large cell carcinoma samples.

Statistical analysis was applied to verify the significance of these results, as described below.

The P value for the difference in the expression levels of Homo sapiens myeloma overexpressed gene transcripts detectable by the above amplicon in Lung non-small cell carcinoma samples versus the normal tissue samples was determined by T test as 9.50e-005. Lung adenocarcinoma samples versus the normal tissue samples was determined by T test as 6.40e-003. The P value for the difference in the expression levels of Homo sapiens myeloma overexpressed gene transcripts detectable by the above amplicon in Lung adenocarcinoma samples, in Lung squamous cell carcinoma samples and lung large cell carcinoma versus the normal tissue samples was determined by T test as 6.40e-003, 8.58e-003 and 1.16e-001, respectively.

Threshold of 5 fold over expression was found to differentiate between non-small cell carcinoma and normal samples with P value of 1.39e-006 as checked by exact Fisher test. Threshold of 5 fold over expression was found to differentiate between adenocarcinoma, squamous cell carcinoma and large cell carcinoma and normal samples with P value of 6.50e-006, 8.45e-005 and 1.82e-004, respectively as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: AA583399_seg1WTF (SEQ ID NO: 362) forward primer; and AA583399_seg1WTR (SEQ ID NO: 363) reverse primer.

The present invention also preferably encompasses any amplicon obtained through the use of any suitable primer pair; for example, for the above experiment, the following amplicon was obtained as a non-limiting illustrative example only of a suitable amplicon: AA583399_seglWT (SEQ ID NO: 364).

Expression of Homo sapiens Myeloma Overexpressed Gene (in a Subset of t(11;14) Positive Multiple Myelomas) (MYEOV) AA583399 Transcripts which are Detectable by Amplicon as Depicted in Sequence Name AA583399_seg1 (SEQ ID NO: 364) in Normal and Cancerous Ovary Tissues

Expression of Homo sapiens myeloma overexpressed gene (in a subset of t(11;14) positive multiple myelomas) (MYEOV) transcripts detectable by or according to seg1—AA583399_seg1 (SEQ ID NO: 364) amplicon and primers AA583399_seg1F (SEQ ID NO: 362) and AA583399_seg1R (SEQ ID NO: 363) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305)), PBGD (GenBank Accession No. BC019323 (SEQ ID NO:336); amplicon—PBGD-amplicon (SEQ ID NO: 302)) and GAPDH (GenBank Accession No. BC026907 (SEQ ID NO:338); GAPDH amplicon (SEQ ID NO: 308)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 45, 46, 71 and 48, Table 1_{—}1 above), to obtain a value of fold up-regulation for each sample relative to the median of the normal PM samples.

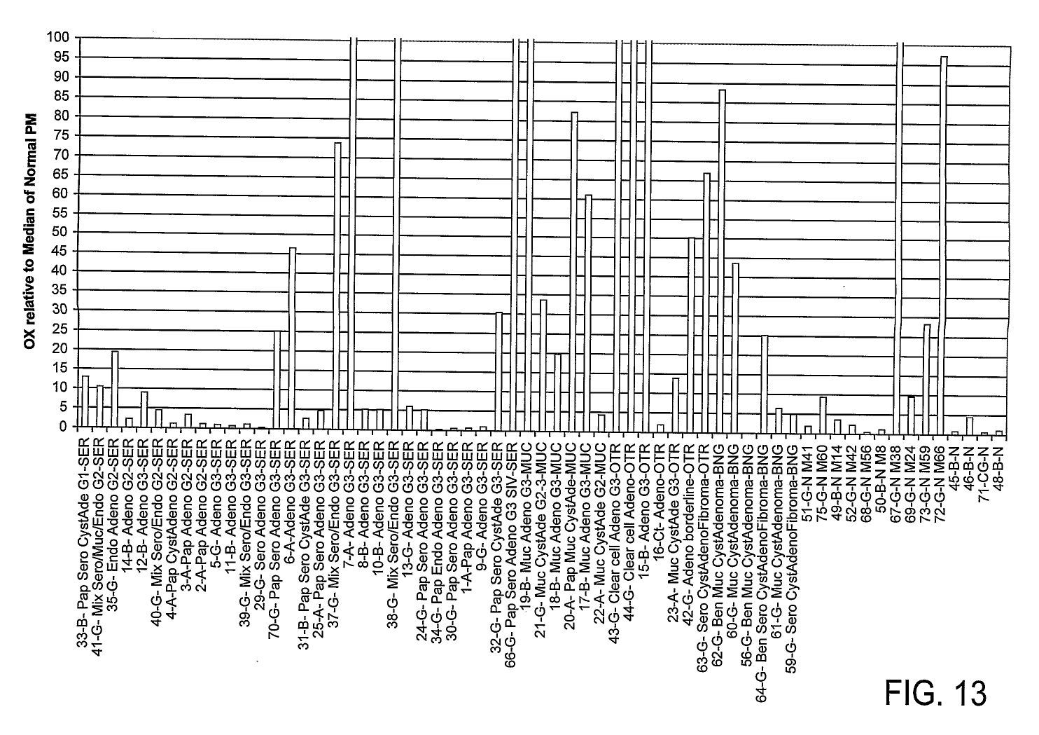

FIG. 13 is a histogram showing over expression of the Homo sapiens myeloma overexpressed gene (in a subset of t(11;14) positive multiple myelomas) (MYEOV) transcripts in cancerous Ovary samples relative to the normal samples for transcripts detectable by or according to AA583399_seg1 (SEQ ID NO: 364).

As is evident from FIG. 13, the expression of Homo sapiens myeloma overexpressed gene (in a subset of t(11;14) positive multiple myelomas) (MYEOV) transcripts detectable by the above amplicon in adenocarcinoma samples was significantly higher than in the non-cancerous samples (sample numbers 45, 46, 71 and 48, Table 1_{—}1 above). Notably an over-expression of at least 5 fold was found in 26 out of 43 adenocarcinoma samples, specifically in 15 out of 30 serous carcinoma samples and in 5 out of 6 mucinous carcinoma samples.

Statistical analysis was applied to verify the significance of these results, as described below.

The P value for the difference in the expression levels of Homo sapiens myeloma overexpressed gene (in a subset of t(11;14) positive multiple myelomas) (MYEOV) transcripts detectable by the above amplicon in Ovary adenocarcinoma samples versus the normal tissue samples was determined by T test as 1.43e-002.

The P value for the difference in the expression levels of Homo sapiens myeloma overexpressed gene (in a subset of t(11;14) positive multiple myelomas) (MYEOV) transcripts detectable by the above amplicon in Ovary serous carcinoma samples versus the normal tissue samples was determined by T test as 3.42e-002. The P value for the difference in the expression levels of Homo sapiens myeloma overexpressed gene (in a subset of t(11;14) positive multiple myelomas) (MYEOV) transcripts detectable by the above amplicon in Ovary mucinous carcinoma samples versus the normal tissue samples was determined by T test as 2.82e-002.

Threshold of 5 fold over expression was found to differentiate between adenocarcinoma and normal samples with P value of 3.36e-002 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: AA583399_seg1F (SEQ ID NO: 362) forward primer; and AA583399_seg1R (SEQ ID NO: 363) reverse primer.

The present invention also preferably encompasses any amplicon obtained through the use of any suitable primer pair; for example, for the above experiment, the following amplicon was obtained as a non-limiting illustrative example only of a suitable amplicon: AA583399_seg1 (SEQ ID NO: 364).

Expression of Homo sapiens Myeloma Overexpressed Gene (in a Subset of t(11;14) Positive Multiple Myelomas) (MYEOV) AA583399 Transcripts which are Detectable by Amplicon as Depicted in Sequence Name AA583399_seg1 (SEQ ID NO: 364) in Different Normal Tissues

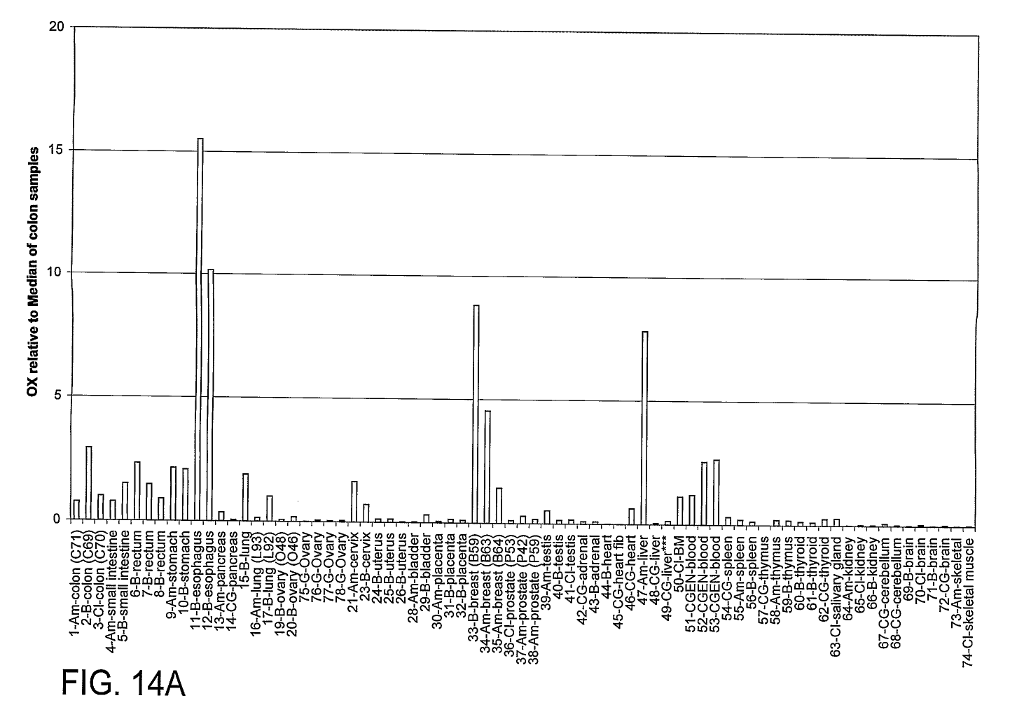

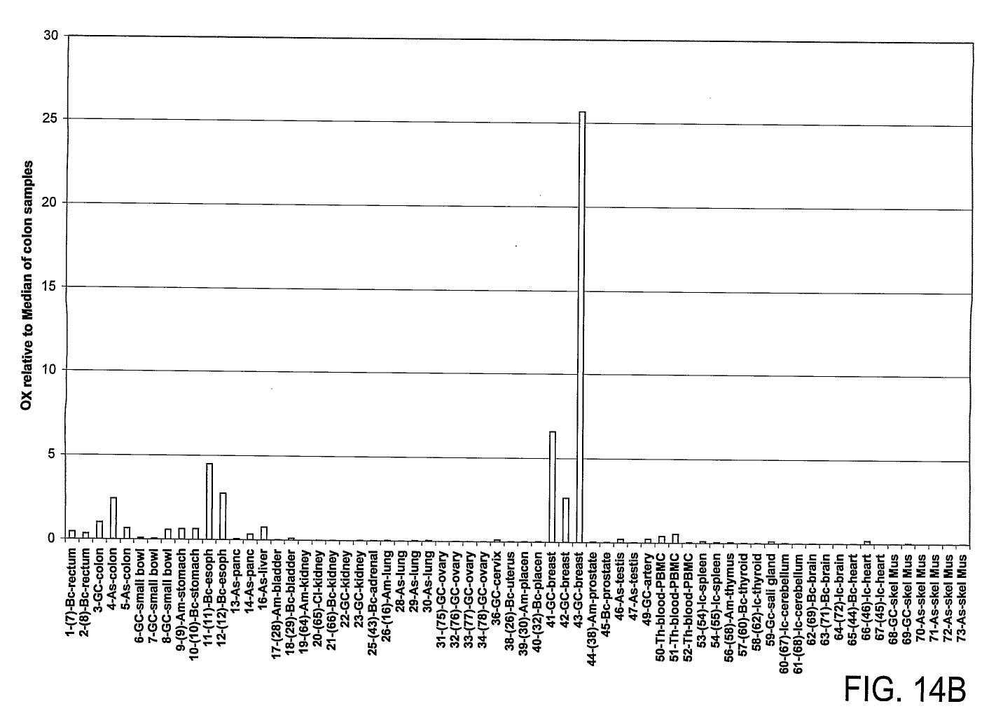

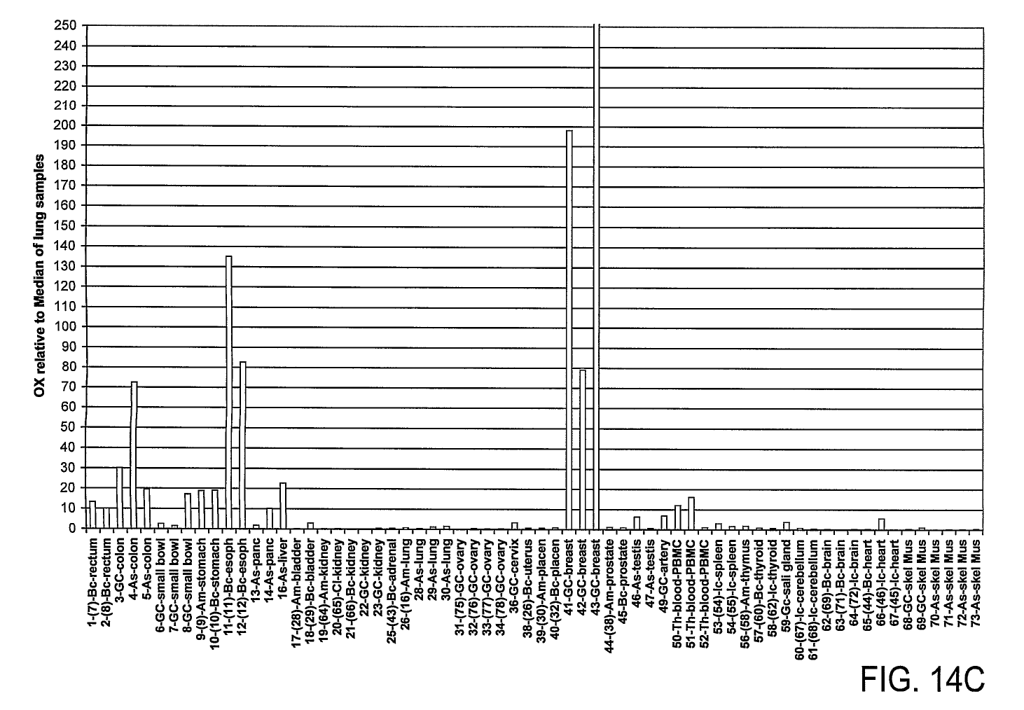

Expression of Homo sapiens myeloma overexpressed gene (in a subset of t(11;14) positive multiple myelomas) (MYEOV) transcripts detectable by or according to seg1—AA583399_seg1 (SEQ ID NO: 364) amplicon and primers AA583399_seg1F (SEQ ID NO: 362) and AA583399_seg1R (SEQ ID NO: 363) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the geometric mean of the quantities of the housekeeping genes. The normalized quantity of each RT sample was then divided by the median of the quantities of the colon samples (sample numbers 1, 2 and 3, Table 1_{—}9 above), to obtain a value of relative expression of each sample relative to the median of the colon samples. FIG. 14A shows the result of expression of Homo sapiens myeloma overexpressed gene (in a subset of t(11;14) positive multiple myelomas) (MYEOV) AA583399 transcripts which are detectable by amplicon as depicted in sequence name AA583399_seg1 (SEQ ID NO: 364) in different normal tissues.

Expression of Homo sapiens myeloma overexpressed gene transcripts detectable by or according to seg1WT—AA583399_seg1WT (SEQ ID NO: 364) amplicon and primers AA583399_seg1WTF (SEQ ID NO: 362) and AA583399_seg1WTR (SEQ ID NO: 363) was further measured by real time PCR using updated panel, as shown in Table 1_{—}10. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the colon samples (sample numbers 3, 4 and 5, Table 1_{—}10 above), to obtain a value of relative expression of each sample relative to the median of the colon samples, as shown in FIG. 14B. The normalized quantity of each RT sample was then divided by the median of the quantities of the lung samples (sample numbers 26, 28, 29 and 30, Table 1_{—}10 above), to obtain a value of relative expression of each sample relative to the median of the lung samples, as shown in FIG. 14C.

Expression of Homo sapiens Myeloma Overexpressed Gene (in a Subset of t(11;14) Positive Multiple Myelomas) (MYEOV) AA583399 Transcripts which are Detectable by Amplicon as Depicted in Sequence Name AA583399 seg17 (SEQ ID NO: 367) in Normal and Cancerous Lung Tissues

Expression of Homo sapiens myeloma overexpressed gene (in a subset of t(11;14) positive multiple myelomas) (MYEOV) transcripts detectable by or according to seg17—AA583399 seg17 (SEQ ID NO: 367) amplicon and primers AA583399 seg17F (SEQ ID NO: 365) and AA583399 seg17R (SEQ ID NO: 366) was measured by real time PCR. In parallel the expression of four housekeeping genes—PB3GD (GenBank Accession No. BC019323 (SEQ ID NO:336); amplicon—PBGD-amplicon (SEQ ID NO: 302)), HPRT1 (GenBank Accession No. NM_{—}000194 (SEQ ID NO:337); amplicon—HPRT1-amplicon (SEQ ID NO: 305) (SEQ ID NO: 305)), Ubiquitin (GenBank Accession No. BC000449 (SEQ ID NO:341); amplicon—Ubiquitin-amplicon (SEQ ID NO: 317)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 47-50, 90-93, 96-99, Table 1_{—}2, above), to obtain a value of fold up-regulation for each sample relative to the median of the normal PM samples.

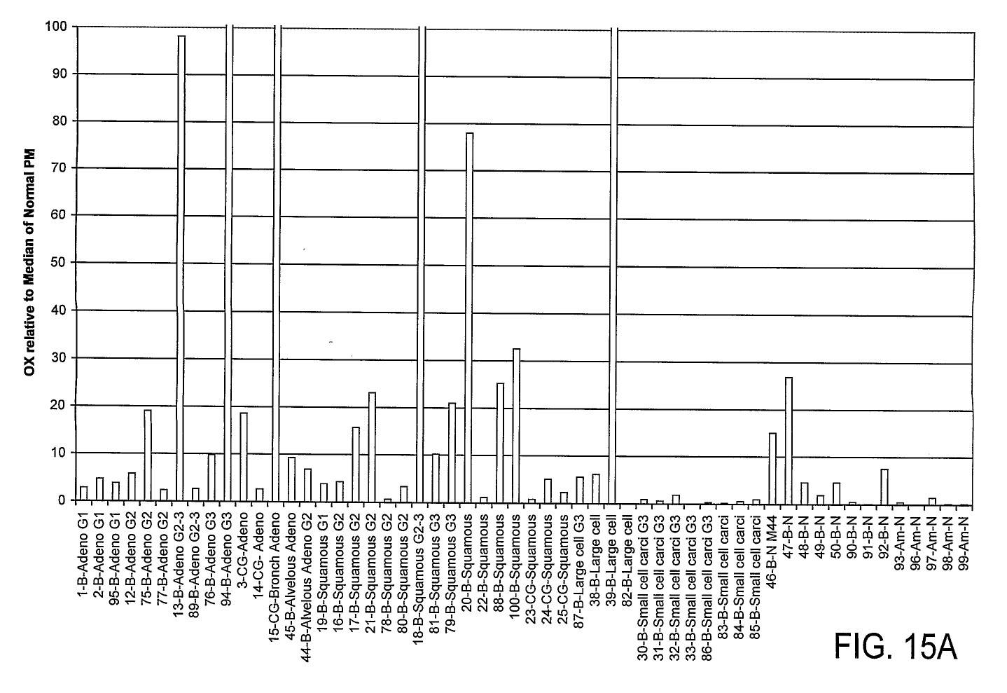

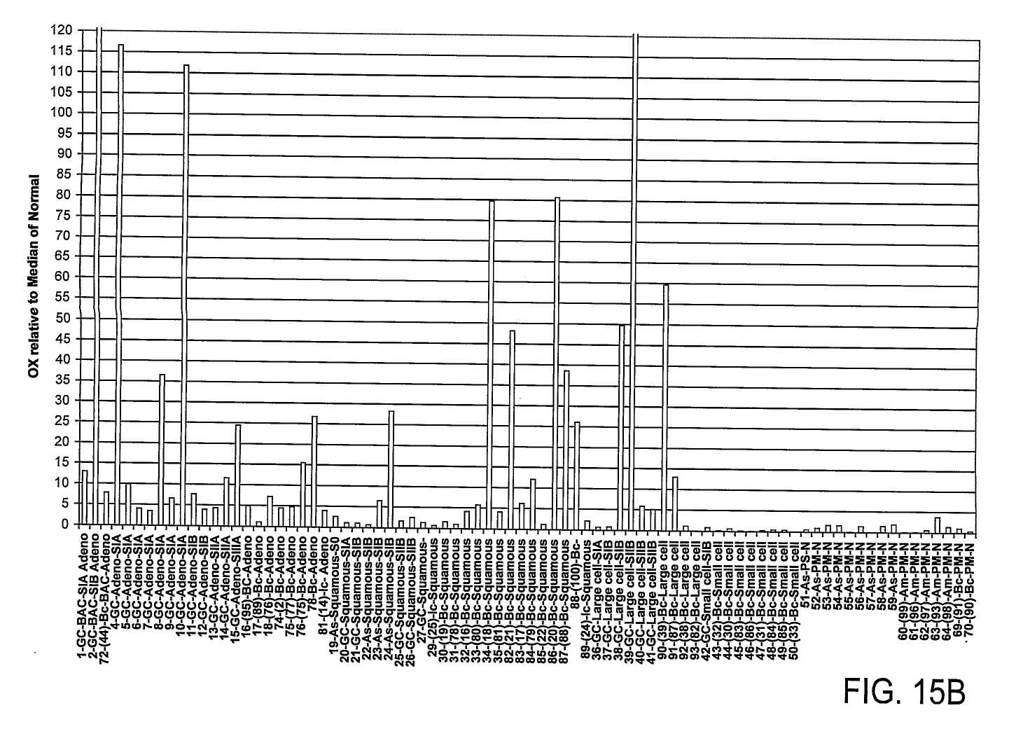

FIG. 15A is a histogram showing over expression of the Homo sapiens myeloma overexpressed gene (in a subset of t(11;14) positive multiple myelomas) (MYEOV) transcripts detectable by or according to AA583399 seg17 (SEQ ID NO: 367) in cancerous lung samples relative to the normal samples.

As is evident from FIG. 15A, the expression of Homo sapiens myeloma overexpressed gene (in a subset of t(11;14) positive multiple myelomas) (MYEOV) transcripts detectable by the above amplicon in cancer samples was significantly higher than in the non-cancerous samples (Sample Nos. 46-50, 90-93, 96-99 Table 1-2). Notably an over-expression of at least 10 fold was found in 5 out of 15 adenocarcinoma samples, 8 out of 16 squamous cell carcinoma samples.

Statistical analysis was applied to verify the significance of these results, as described below.

The P value for the difference in the expression levels of Homo sapiens myeloma overexpressed gene (in a subset of t(11;14) positive multiple myelomas) (MYEOV) transcripts detectable by the above amplicon in Lung adenocarcinoma samples versus the normal tissue samples was determined by T test as 6.15E-02.

The P value for the difference in the expression levels of Homo sapiens myeloma overexpressed gene (in a subset of t(11;14) positive multiple myelomas) (MYEOV) transcripts detectable by the above amplicon in Lung squamous cell carcinoma samples versus the normal tissue samples was determined by T test as 4.4E-02.

The P value for the difference in the expression levels of Homo sapiens myeloma overexpressed gene (in a subset of t(11;14) positive multiple myelomas) (MYEOV) transcripts detectable by the above amplicon in all Lung Non-small cells carcinoma samples versus the normal tissue samples was determined by T test as 4.22E-03.

Threshold of 10 fold overexpression was found to differentiate between all Lung Non-small cell carcinoma and normal samples with P value of 3.63E-02 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: AA583399 seg17F (SEQ ID NO: 365) forward primer; and AA583399 seg17R (SEQ ID NO: 366) reverse primer.

The present invention also preferably encompasses any amplicon obtained through the use of any suitable primer pair; for example, for the above experiment, the following amplicon was obtained as a non-limiting illustrative example only of a suitable amplicon: AA583399 seg17 (SEQ ID NO: 367).

Expression of Homo sapiens myeloma overexpressed gene (in a subset of t(11;14) positive multiple myelomas) (MYEOV) transcripts detectable by or according to seg17—AA583399_seg17 (SEQ ID NO: 367) amplicon and primers AA583399_seg17F (SEQ ID NO: 365) and AA583399_seg17R (SEQ ID NO: 366) was further measured by real time PCR using enlarged panel as shown in Table 1_{—}6. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 51, 52, 53, 54, 55, 56, 57, 58, 59, 60, 61, 62, 63, 64, 69 and 70, Table 1_{—}6 above), to obtain a value of fold up-regulation for each sample relative to the median of the normal samples.

FIG. 15B is a histogram showing over expression of the above-indicated Homo sapiens myeloma overexpressed gene (in a subset of t(11;14) positive multiple myelomas) (MYEOV) transcripts in cancerous Lung samples relative to the normal samples.

As is evident from FIG. 15B, the expression of Homo sapiens myeloma overexpressed gene (in a subset of t(11;14) positive multiple myelomas) (MYEOV) transcripts detectable by the above amplicon in non-small cell carcinoma samples was significantly higher than in the non-cancerous samples (sample numbers 51, 52, 53, 54, 55, 56, 57, 58, 59, 60, 61, 62, 63, 64, 69 and 70, Table 1_{—}6 above). Notably an over-expression of at least 5 fold was found in 30 out of 57 non-small cell carcinoma samples. In 14 out of 23 adenocarcinoma samples, in 10 out of 24 squamous cell carcinoma samples and in 6 out of 10 large cell carcinoma samples.

Statistical analysis was applied to verify the significance of these results, as described below.

The P value for the difference in the expression levels of Homo sapiens myeloma overexpressed gene (in a subset of t(11;14) positive multiple myelomas) (MYEOV) transcripts detectable by the above amplicon in Lung non-small cell carcinoma samples versus the normal tissue samples was determined by T test as 5.02e-005. The P value for the difference in the expression levels of Homo sapiens myeloma overexpressed gene (in a subset of t(11;14) positive multiple myelomas) (MYEOV) transcripts detectable by the above amplicon in Lung adenocarcinoma samples, Lung squamous cell carcinoma samples and lung large cell carcinoma versus the normal tissue samples was determined by T test as 8.52e-003, 9.45e-003 and 9.18e-002 respectively.

Threshold of 5 fold over expression was found to differentiate between non-small cell carcinoma and normal samples with P value of 5.03e-005 as checked by exact Fisher test. Threshold of 5 fold over expression was found to differentiate between adenocarcinoma, squamous cell carcinoma and large cell carcinoma and normal samples with P value of 5.42e-005, 2.31e-003 and 9.12e-004 respectively as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: AA583399_seg17F (SEQ ID NO: 365) forward primer; and AA583399_seg17R (SEQ ID NO: 366) reverse primer.

The present invention also preferably encompasses any amplicon obtained through the use of any suitable primer pair; for example, for the above experiment, the following amplicon was obtained as a non-limiting illustrative example only of a suitable amplicon: AA583399_seg17 (SEQ ID NO: 367).

Expression of Homo sapiens Myeloma Overexpressed Gene (in a Subset of t(11;14) Positive Multiple Myelomas) (MYEOV) AA583399 Transcripts which are Detectable by Amplicon as Depicted in Sequence Name AA583399_seg17 (SEQ ID NO: 367) in Normal and Cancerous Ovary Tissues

Expression of Homo sapiens myeloma overexpressed gene (in a subset of t(11;14) positive multiple myelomas) (MYEOV) transcripts detectable by or according to seg17—AA583399_seg17 (SEQ ID NO: 367) amplicon and primers AA583399_seg17F (SEQ ID NO: 365) and AA583399_seg17R (SEQ ID NO: 366) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and GAPDH (GenBank Accession No. BC026907 (SEQ ID NO:338); GAPDH amplicon (SEQ ID NO: 308)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 45, 46 and 48, Table 1_{—}1 above), to obtain a value of fold up-regulation for each sample relative to the median of the normal PM samples.

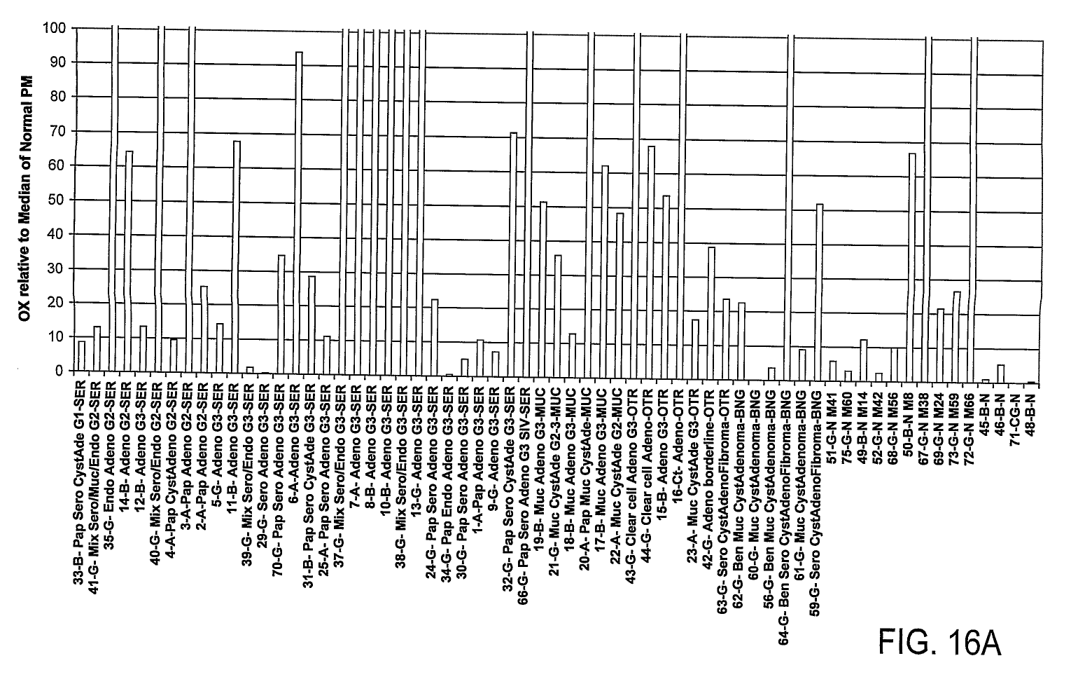

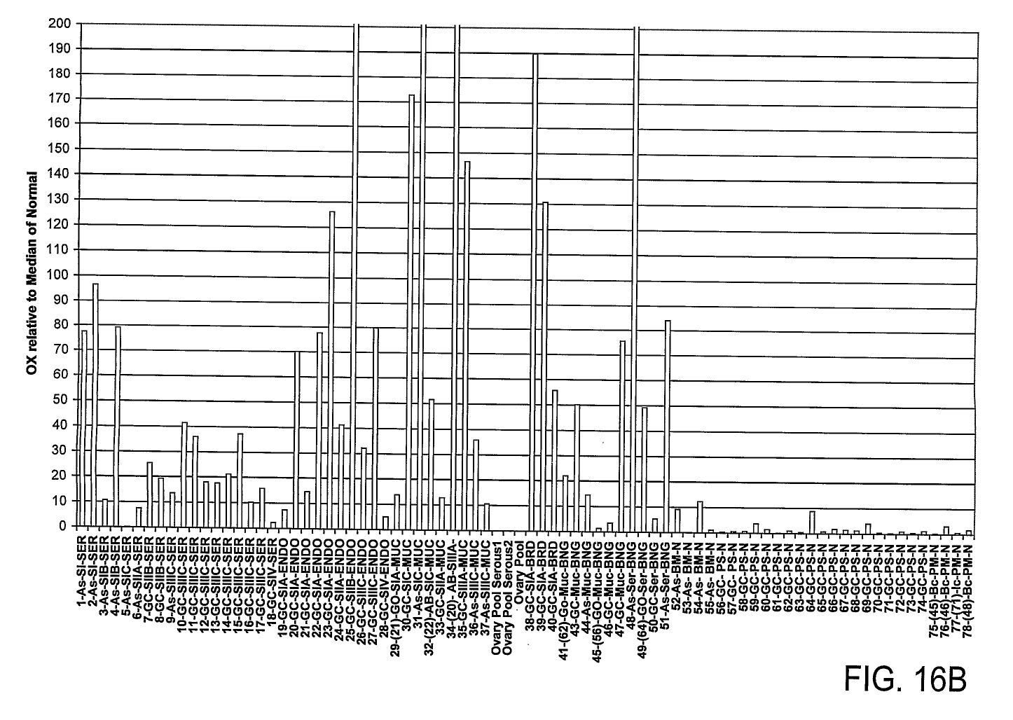

FIG. 16A is a histogram showing over expression of the Homo sapiens myeloma overexpressed gene (in a subset of t(11;14) positive multiple myelomas) (MYEOV) transcripts detectable by or according to AA583399_seg17 (SEQ ID NO: 367) in cancerous ovary samples relative to the normal samples.

As is evident from FIG. 16A, the expression of Homo sapiens myeloma overexpressed gene (in a subset of t(11;14) positive multiple myelomas) (MYEOV) transcripts detectable by the above amplicon in adenocarcinoma samples was significantly higher than in the non-cancerous samples (sample numbers 45, 46 and 48, Table 1_{—}1 above). Notably an over-expression of at least 5 fold was found in 39 out of 43 adenocarcinoma samples, specifically in 26 out of 30 serous carcinoma samples and in 6 out of 6 mucinous carcinoma samples.

Statistical analysis was applied to verify the significance of these results, as described below.

The P value for the difference in the expression levels of Homo sapiens myeloma overexpressed gene (in a subset of t(11;14) positive multiple myelomas) (MYEOV) transcripts detectable by the above amplicon in Ovary adenocarcinoma samples versus the normal tissue samples was determined by T test as 2.31e-003.

The P value for the difference in the expression levels of Homo sapiens myeloma overexpressed gene (in a subset of t(11;14) positive multiple myelomas) (MYEOV) transcripts detectable by the above amplicon in Ovary serous carcinoma samples versus the normal tissue samples was determined by T test as 7.93e-003. Threshold of 5 fold over expression was found to differentiate between adenocarcinoma and normal samples with P value of 4.05e-002 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: AA583399_seg17F (SEQ ID NO: 365) forward primer; and AA583399_seg17R (SEQ ID NO: 366) reverse primer.

The present invention also preferably encompasses any amplicon obtained through the use of any suitable primer pair; for example, for the above experiment, the following amplicon was obtained as a non-limiting illustrative example only of a suitable amplicon: AA583399_seg17 (SEQ ID NO: 367).

Expression of Homo sapiens myeloma overexpressed gene transcripts detectable by or according to seg17-AA583399_seg17 (SEQ ID NO: 367) amplicon and primers AA583399_seg17F (SEQ ID NO: 365) and AA583399_seg17R (SEQ ID NO: 366) was further measured by real time PCR using enlarged panel as shown in Table 1_{—}5. In parallel the expression of three housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)) and G6PD (GenBank Accession No. NM_{—}000402 (SEQ ID NO:339); G6PD amplicon (SEQ ID NO: 311)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above), to obtain a value of fold up-regulation for each sample relative to the median of the normal samples.

FIG. 16B is a histogram showing over expression of the above-indicated Homo sapiens myeloma overexpressed gene transcripts in cancerous Ovary samples relative to the normal samples.

As is evident from FIG. 16B, the expression of Homo sapiens myeloma overexpressed gene transcripts detectable by the above amplicon in adenocarcinoma samples was significantly higher than in the non-cancerous samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above). Notably an over-expression of at least 10 fold was found in 15 out of 18 serous carcinoma samples, in 9 out of 9 mucinous carcinoma samples and in 8 out of 10 Endometroid samples.

Statistical analysis was applied to verify the significance of these results, as described below.

The P value for the difference in the expression levels of Homo sapiens myeloma overexpressed gene transcripts detectable by the above amplicon in Ovary adenocarcinoma samples versus the normal tissue samples was determined by T test as 1.24e-004. The P value for the difference in the expression levels of Homo sapiens myeloma overexpressed gene transcripts detectable by the above amplicon in Ovary serous carcinoma samples versus the normal tissue samples was determined by T test as 6.72e-004. The P value for the difference in the expression levels of Homo sapiens myeloma overexpressed gene transcripts detectable by the above amplicon in Ovary mucinous carcinoma samples versus the normal tissue samples was determined by T test as 2.86e-002. The P value for the difference in the expression levels of Homo sapiens myeloma overexpressed gene transcripts detectable by the above amplicon in Ovary endometroid samples versus the normal tissue samples was determined by T test as 1.03e-002.

Threshold of 10 fold over expression was found to differentiate between adenocarcinoma and normal samples with P value of 6.65e-012 as checked by exact Fisher test. Threshold of 10 fold over expression was found to differentiate between serous carcinoma and normal samples with P value of 3.42e-008 as checked by exact Fisher test. Threshold of 10 fold over expression was found to differentiate between mucinous carcinoma and normal samples with P value of 1.06e-007 as checked by exact Fisher test. Threshold of 10 fold over expression was found to differentiate between endometroid and normal samples with P value of 9.81 e-006 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: AA583399_seg17F (SEQ ID NO: 365) forward primer; and AA583399_seg17R (SEQ ID NO: 366) reverse primer.

The present invention also preferably encompasses any amplicon obtained through the use of any suitable primer pair; for example, for the above experiment, the following amplicon was obtained as a non-limiting illustrative example only of a suitable amplicon: AA583399_seg17 (SEQ ID NO: 367).

Expression of Homo sapiens Myeloma Overexpressed Gene (in a Subset of t(11;14) Positive Multiple Myelomas) (MYEOV) AA583399 Transcripts which are Detectable by Amplicon as Depicted in Sequence Name AA583399 seg17 (SEQ ID NO: 367) in Different Normal Tissues

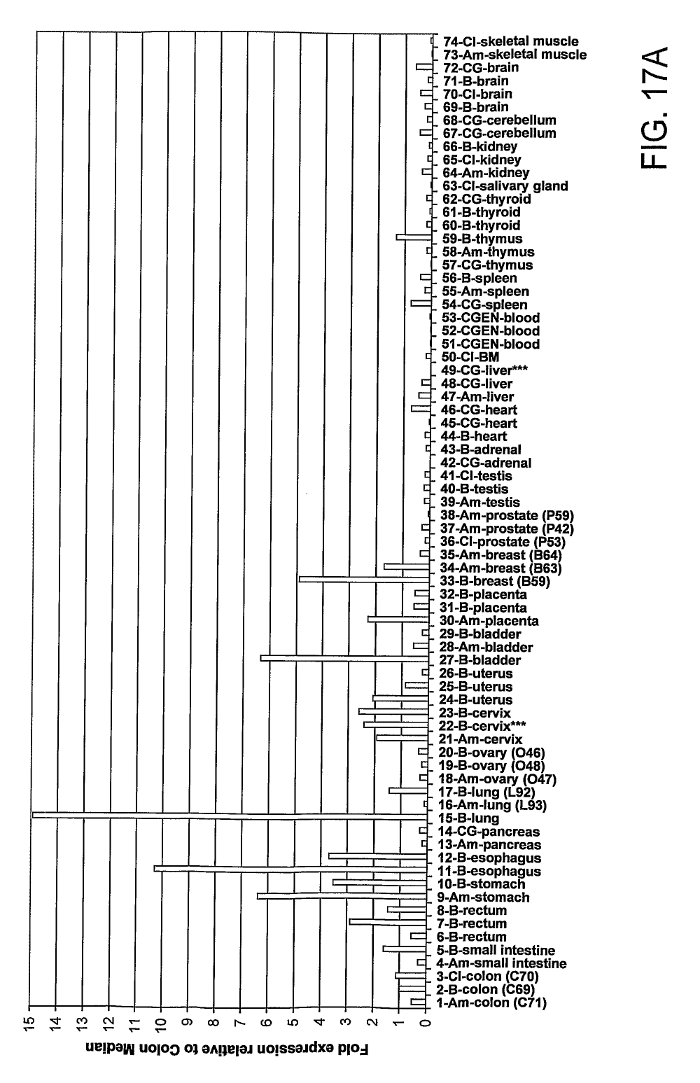

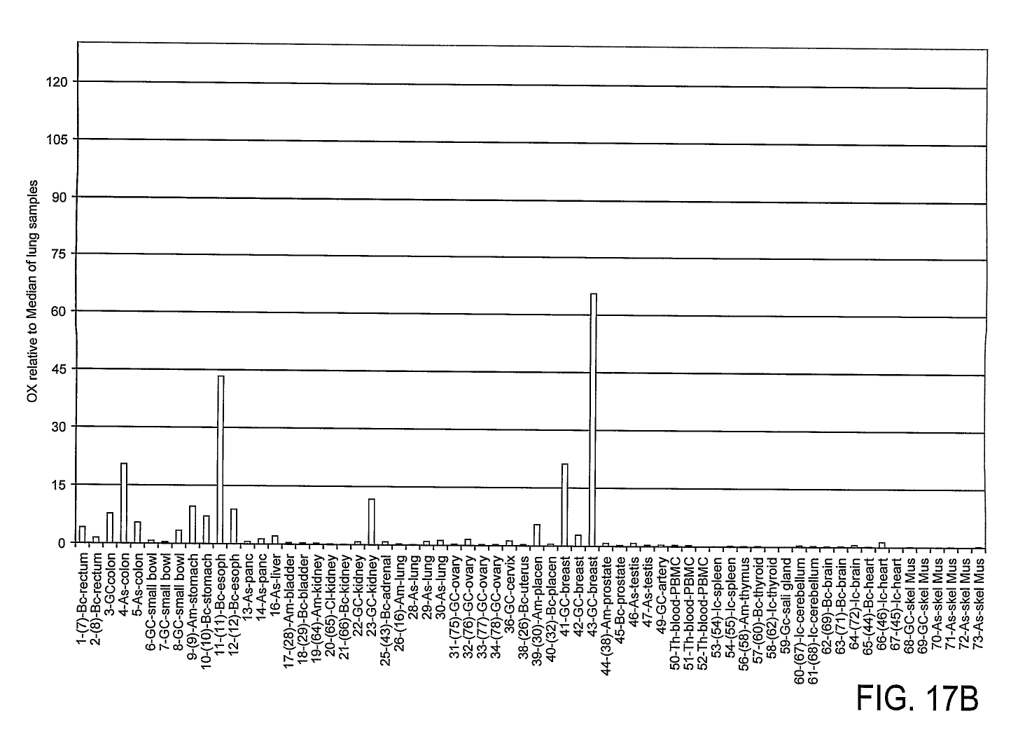

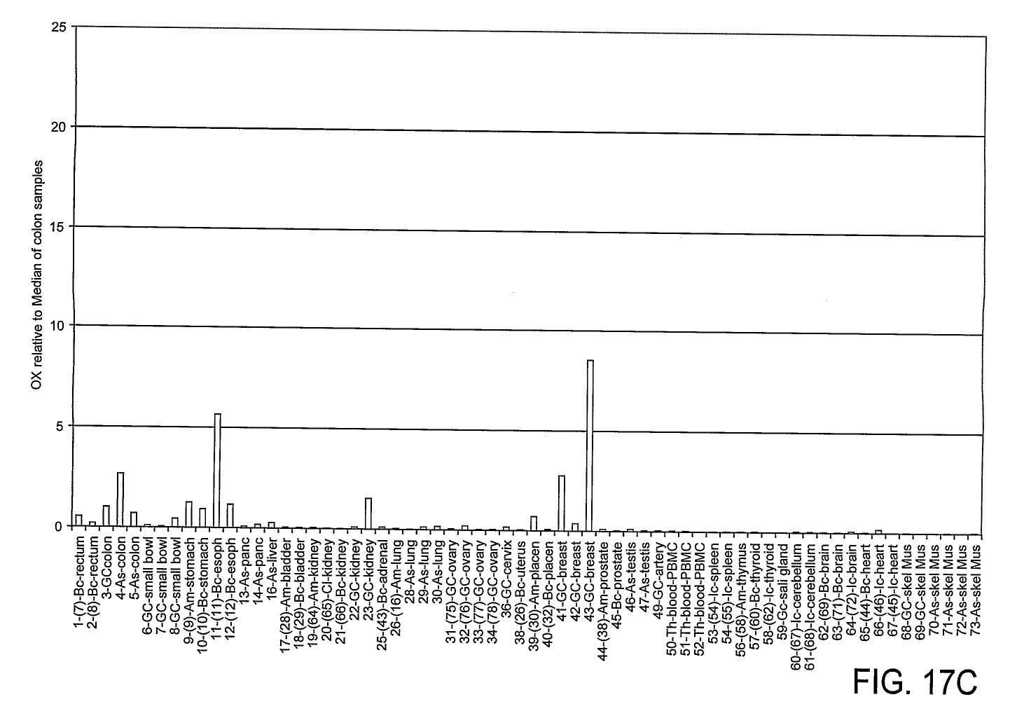

Expression of Homo sapiens myeloma overexpressed gene (in a subset of t(11;14) positive multiple myelomas) (MYEOV) transcripts detectable by or according to AA583399 seg17 (SEQ ID NO: 367) amplicon and primers: AA583399 seg17F (SEQ ID NO: 365) and AA583399 seg17R (SEQ ID NO: 366) was measured by real time PCR. In parallel the expression of four housekeeping genes—RPL19 (GenBank Accession No. NM_{—}000981 (SEQ ID NO:342); RPL19 amplicon (SEQ ID NO: 320)), TATA box (GenBank Accession No. NM_{—}003194 (SEQ ID NO:343); TATA amplicon (SEQ ID NO: 323)), Ubiquitin (GenBank Accession No. BC000449 (SEQ ID NO:341); amplicon—Ubiquitin-amplicon (SEQ ID NO: 317)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the geometric mean of the quantities of the housekeeping genes. The normalized quantity of each RT sample was then divided by the median of the quantities of the colon samples (Sample Nos. 69-71 Table 1_{—}9), to obtain a value of relative expression of each sample relative to the median of the colon samples. FIG. 17A shows expression of Homo sapiens myeloma overexpressed gene (in a subset of t(11;14) positive multiple myelomas) (MYEOV) AA583399 transcripts which are detectable by amplicon as depicted in sequence name AA583399 seg17 (SEQ ID NO: 367) in different normal tissues.

Expression of Homo sapiens myeloma overexpressed gene (in a subset of t(11;14) positive multiple myelomas) (MYEOV) transcripts detectable by or according to seg17—AA583399_seg17 (SEQ ID NO: 367) amplicon and primers AA583399_seg17F (SEQ ID NO: 365) and AA583399_seg17R (SEQ ID NO: 366) was further measured by real time PCR using updated panel, shown in Table 1_{—}10. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), and TATA box (GenBank Accession No. NM_{—}003194 (SEQ ID NO:343); TATA amplicon (SEQ ID NO: 323)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the lung samples (sample numbers 26, 27, 28, 29 and 30, Table 1_{—}10 above), to obtain a value of relative expression of each sample relative to the median of the lung samples, as presented in FIG. 17B, or to the median of the quantities of the colon samples (sample numbers 3-5, Table 1_{—}10 above), to obtain a value of relative expression of each sample relative to the median of the colon samples, as presented in FIG. 17C.

Expression of Homo sapiens Myeloma Overexpressed Gene (in a Subset of t(11;14) Positive Multiple Myelomas) (MYEOV) AA583399 Transcripts which are Detectable by Amplicon as Depicted in Sequence Name AA583399_seg30-32 (SEQ ID NO: 370) in Normal and Cancerous Lung Tissues

Expression of Homo sapiens myeloma overexpressed gene (in a subset of t(11;14) positive multiple myelomas) (MYEOV) transcripts detectable by or according to seg30-32—AA583399_seg30-32 (SEQ ID NO: 370) amplicon and primers AA583399_seg30-32F (SEQ ID NO: 368) and AA583399_seg30-32R (SEQ ID NO: 369) was measured by real time PCR. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 47, 48, 49, 50, 90, 91, 92, 93, 96, 97 and 98, Table 1_{—}2 above), to obtain a value of fold up-regulation for each sample relative to the median of the normal PM samples.

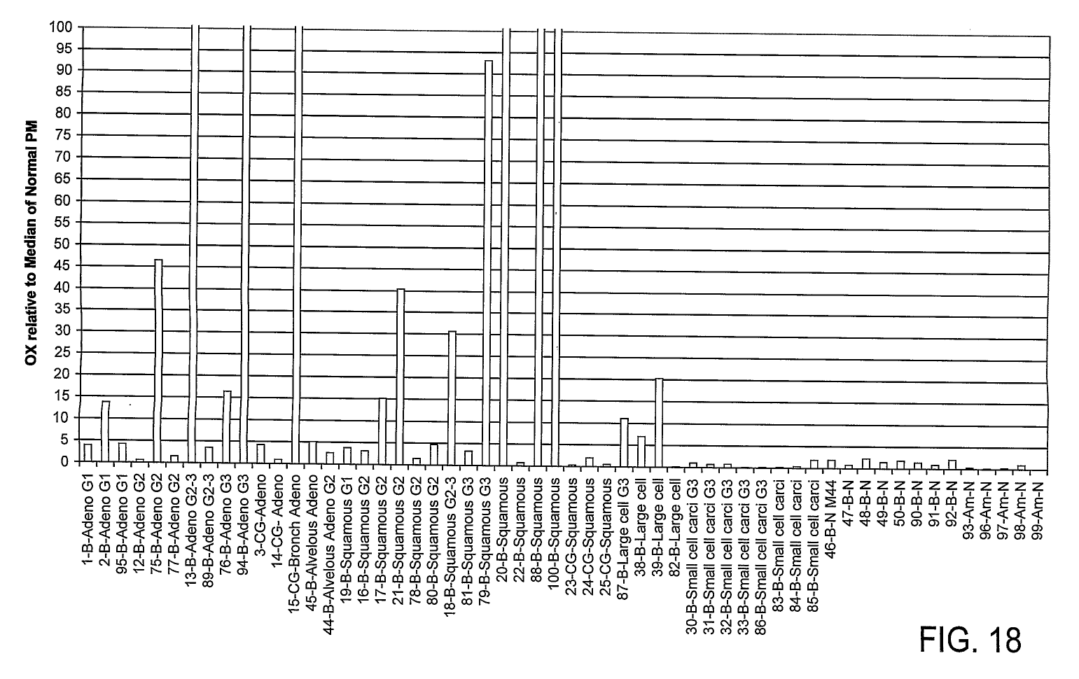

FIG. 18 is a histogram showing over expression of the Homo sapiens myeloma overexpressed gene (in a subset of t(11;14) positive multiple myelomas) (MYEOV) transcripts detectable by or according to AA583399_seg30-32 (SEQ ID NO: 370) in cancerous Lung samples relative to the normal samples.

As is evident from FIG. 18, the expression of Homo sapiens myeloma overexpressed gene (in a subset of t(11;14) positive multiple myelomas) (MYEOV) transcripts detectable by the above amplicon in adenocarcinoma, squamous cell carcinoma, large cell carcinoma was significantly higher than in the non-cancerous samples (sample numbers 47, 48, 49, 50, 90, 91, 92, 93, 96, 97 and 98, Table 1_{—}2 above). Notably an over-expression of at least 5 fold was found in 17 out of 35 non-small cell carcinoma samples, specifically in 7 out of 15 adenocarcinoma samples, in 7 out of 16 squamous cell carcinoma samples and in 3 out of 4 large cell carcinoma samples.

Statistical analysis was applied to verify the significance of these results, as described below.

The P value for the difference in the expression levels of Homo sapiens myeloma overexpressed gene (in a subset of t(11;14) positive multiple myelomas) (MYEOV) transcripts detectable by the above amplicon in Lung non-small cell carcinoma samples versus the normal tissue samples was determined by T test as 1.60e-002.

Threshold of 5 fold over expression was found to differentiate between non-small cell carcinoma and normal samples with P value of 2.59e-003 as checked by exact Fisher test. Threshold of 5 fold over expression was found to differentiate between adenocarcinoma and normal samples with P value of 9.78e-003 as checked by exact Fisher test. Threshold of 5 fold over expression was found to differentiate between squamous cell carcinoma and normal samples with P value of 1.29e-002 as checked by exact Fisher test. Threshold of 5 fold over expression was found to differentiate between large cell carcinoma and normal samples with P value of 8.79e-003 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: AA583399_seg30-32F (SEQ ID NO: 368) forward primer; and AA583399_seg30-32R (SEQ ID NO: 369) reverse primer.

The present invention also preferably encompasses any amplicon obtained through the use of any suitable primer pair; for example, for the above experiment, the following amplicon was obtained as a non-limiting illustrative example only of a suitable amplicon: AA583399_seg30-32 (SEQ ID NO: 370).

Expression of Homo sapiens Myeloma Overexpressed Gene (in a Subset of t(11;14) Positive Multiple Myelomas) (MYEOV) AA583399 Transcripts which are Detectable by Amplicon as Depicted in Sequence Name AA583399_seg30-32 (SEQ ID NO: 370) in Normal and Cancerous Ovary Tissues

Expression of Homo sapiens myeloma overexpressed gene (in a subset of t(11;14) positive multiple myelomas) (MYEOV) transcripts detectable by or according to seg30-32—AA583399_seg30-32 (SEQ ID NO: 370) amplicon and primers AA583399_seg30-32F (SEQ ID NO: 368) and AA583399_seg30-32R (SEQ ID NO: 369) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and GAPDH (GenBank Accession No. BC026907 (SEQ ID NO:338); GAPDH amplicon (SEQ ID NO: 308)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 45, 46, 71 and 48, Table 1_{—}1 above), to obtain a value of fold up-regulation for each sample relative to the median of the normal PM samples.

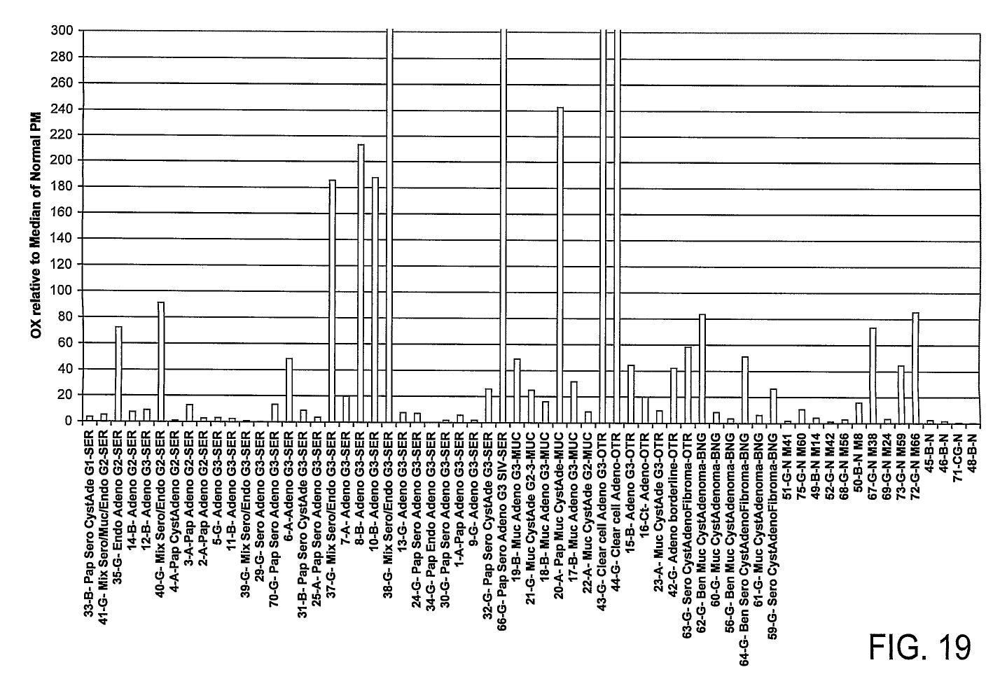

FIG. 19 is a histogram showing over expression of the Homo sapiens myeloma overexpressed gene (in a subset of t(11;14) positive multiple myelomas) (MYEOV) transcripts detectable by or according to AA583399_seg30-32 (SEQ ID NO: 370) in cancerous Ovary samples relative to the normal samples.

As is evident from FIG. 19, the expression of Homo sapiens myeloma overexpressed gene (in a subset of t(11;14) positive multiple myelomas) (MYEOV) transcripts detectable by the above amplicon in adenocarcinoma samples was significantly higher than in the non-cancerous samples (sample numbers 45, 46, 71 and 48, Table 11 above). Notably an over-expression of at least 5 fold was found in 32 out of 43 adenocarcinoma samples, specifically in 19 out of 30 serous carcinoma samples and in 6 out of 6 mucinous carcinoma samples.

Statistical analysis was applied to verify the significance of these results, as described below.

The P value for the difference in the expression levels of Homo sapiens myeloma overexpressed gene (in a subset of t(11;14) positive multiple myelomas) (MYEOV) transcripts detectable by the above amplicon in Ovary adenocarcinoma samples versus the normal tissue samples was determined by T test as 8.08e-003.

The P value for the difference in the expression levels of Homo sapiens myeloma overexpressed gene (in a subset of t(11;14) positive multiple myelomas) (MYEOV) transcripts detectable by the above amplicon in Ovary serous carcinoma samples versus the normal tissue samples was determined by T test as 2.55e-002. Threshold of 5 fold over expression was found to differentiate between adenocarcinoma and normal samples with P value of 7.65e-003 as checked by exact Fisher test. Threshold of 5 fold over expression was found to differentiate between serous carcinoma and normal samples with P value of 2.94e-002 as checked by exact Fisher test. Threshold of 5 fold over expression was found to differentiate between mucinous carcinoma and normal samples with P value of 4.76e-003 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: AA583399_seg30-32P (SEQ ID NO: 368) forward primer; and AA583399_seg30-32R (SEQ ID NO: 369) reverse primer.

The present invention also preferably encompasses any amplicon obtained through the use of any suitable primer pair; for example, for the above experiment, the following amplicon was obtained as a non-limiting illustrative example only of a suitable amplicon: AA583399_seg30-32 (SEQ ID NO: 370).

Expression of Homo sapiens Myeloma Overexpressed Gene (in a Subset of t(11; 14) Positive Multiple Myelomas) (MYEOV) AA583399 Transcripts which are Detectable by Amplicon as Depicted in Sequence Name AA583399 seg30-32 (SEQ ID NO: 370) in Different Normal Tissues

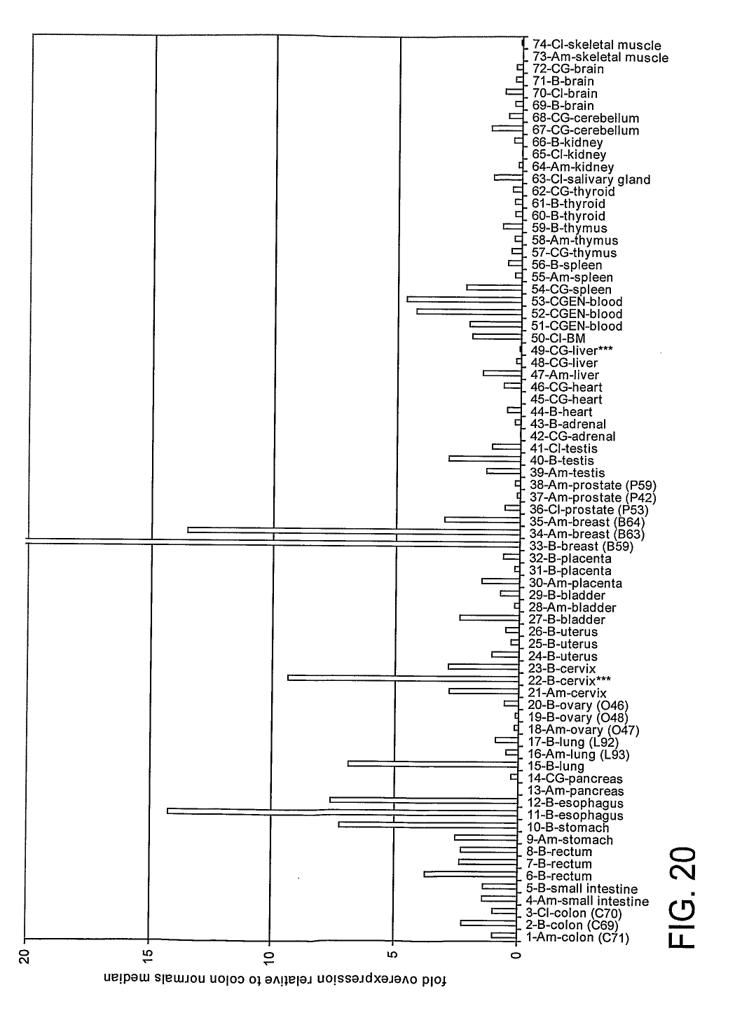

Expression of Homo sapiens myeloma overexpressed gene (in a subset of t(11;14) positive multiple myelomas) (MYEOV) transcripts detectable by or according to AA583399 seg30-32 (SEQ ID NO: 370) amplicon(s) and primers: AA583399 seg30-32F (SEQ ID NO: 368) and AA583399 seg30-32R (SEQ ID NO: 369) was measured by real time PCR. In parallel the expression of four housekeeping genes—RPL19 (GenBank Accession No. NM_{—}000981 (SEQ ID NO:342); RPL19 amplicon (SEQ ID NO: 320)), TATA box (GenBank Accession No. NM_{—}003194 (SEQ ID NO:343); TATA amplicon (SEQ ID NO: 323)), Ubiquitin (GenBank Accession No. BC000449 (SEQ ID NO:341); amplicon—Ubiquitin-amplicon (SEQ ID NO: 317)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the geometric mean of the quantities of the housekeeping genes. The normalized quantity of each RT sample was then divided by the median of the quantities of the colon samples (Sample Nos. 69-71 Table 1_{—}9), to obtain a value of relative expression of each sample relative to the median of the colon samples. FIG. 20 shows Expression of Homo sapiens myeloma overexpressed gene (in a subset of t(11;14) positive multiple myelomas) (MYEOV) AA583399 transcripts which are detectable by amplicon as depicted in sequence name AA583399_seg30-32 (SEQ ID NO: 370) in different normal tissues.

Expression of Homo sapiens Myeloma Overexpressed Gene (in a Subset of t(11;14) Positive Multiple Myelomas) (MYEOV) AA583399 Transcripts which are Detectable by Amplicon as Depicted in Sequence Name AA583399_seg17 (SEQ ID NO: 367) in Normal and Cancerous Colon Tissues

Expression of Homo sapiens myeloma overexpressed gene (in a subset of t(11;14) positive multiple myelomas) (MYEOV) transcripts detectable by or according to seg17—AA583399_seg17 (SEQ ID NO: 367) amplicon and primers AA583399_seg17F (SEQ ID NO: 365) and AA583399_seg17R (SEQ ID NO: 366) was measured by real time PCR. In parallel the expression of three housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), and G6PD (GenBank Accession No. NM_{—}000402 (SEQ ID NO:339); G6PD amplicon (SEQ ID NO: 311)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 42, 43, 44, 45, 46, 47, 48, 49, 50, 51, 53, 54, 55, 56, 57, 58, 59, 60, 61, 62, 63, 64, 65, 66, 67, 68, 69 and 70, Table 1_{—}8 above), to obtain a value of fold up-regulation for each sample relative to the median of the normal samples.

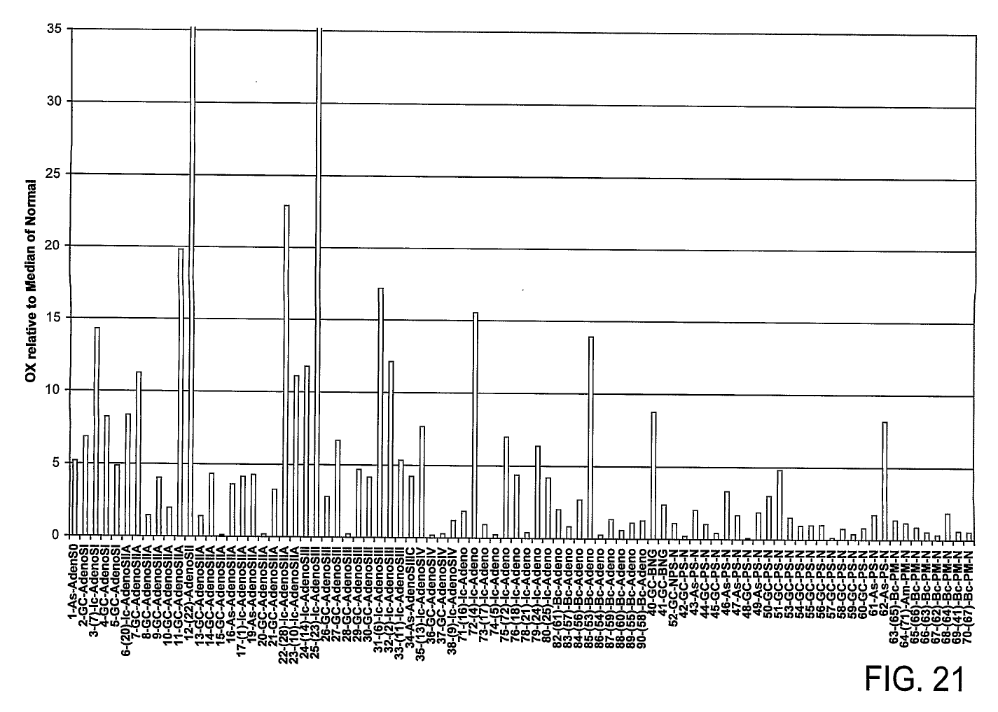

FIG. 21 is a histogram showing over expression of the above-indicated Homo sapiens myeloma overexpressed gene (in a subset of t(11;14) positive multiple myelomas) (MYEOV) transcripts in cancerous Colon samples relative to the normal samples.

As is evident from FIG. 21, the expression of Homo sapiens myeloma overexpressed gene (in a subset of t(11;14) positive multiple myelomas) (MYEOV) transcripts detectable by the above amplicon in cancer samples was significantly higher than in the non-cancerous samples (sample numbers 42, 43, 44, 45, 46, 47, 48, 49, 50, 51, 53, 54, 55, 56, 57, 58, 59, 60, 61, 62, 63, 64, 65, 66, 67, 68, 69 and 70, Table 1_{—}8 above). Notably an over-expression of at least 5 fold was found in 21 out of 55 adenocarcinoma samples.

Statistical analysis was applied to verify the significance of these results, as described below.

The P value for the difference in the expression levels of Homo sapiens myeloma overexpressed gene (in a subset of t(11;14) positive multiple myelomas) (MYEOV) transcripts detectable by the above amplicon in Colon cancer samples versus the normal tissue samples was determined by T test as 8.23e-004.

Threshold of 5 fold over expression was found to differentiate between cancer and normal samples with P value of 3.59e-004 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: AA583399_seg17F (SEQ ID NO: 365) forward primer; and AA583399_seg17R (SEQ ID NO: 366) reverse primer.

The present invention also preferably encompasses any amplicon obtained through the use of any suitable primer pair; for example, for the above experiment, the following amplicon was obtained as a non-limiting illustrative example only of a suitable amplicon: AA583399_seg17 (SEQ ID NO: 367).

Expression of Homo sapiens Myeloma Overexpressed Gene AA583399 Transcripts which are Detectable by Amplicon as Depicted in Sequence Name AA583399_seg17 (SEQ ID NO: 367) in Normal and Cancerous Breast Tissues

Expression of Homo sapiens myeloma overexpressed gene transcripts detectable by or according to seg17-AA583399_seg17 (SEQ ID NO: 367) amplicon and primers AA583399_seg17F (SEQ ID NO: 365) and AA583399_seg17R (SEQ ID NO: 366) was measured by real time PCR. In parallel the expression of four housekeeping genes—G6PD (GenBank Accession No. NM_{—}000402 (SEQ ID NO:339); G6PD amplicon (SEQ ID NO: 311)), RPL19 (GenBank Accession No. NM_{—}000981 (SEQ ID NO:342); RPL19 amplicon (SEQ ID NO: 320)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 43, 45, 46, 47, 48, 49, 50, 51, 52, 54, 56, 58, 59, 60, 61, 62, 63, 64, 65, 66, 67, 68 and 69, Table 1_{—}7 above). Then the reciprocal of this ratio was calculated, to obtain a value of fold down-regulation for each sample relative to the median of the normal samples.

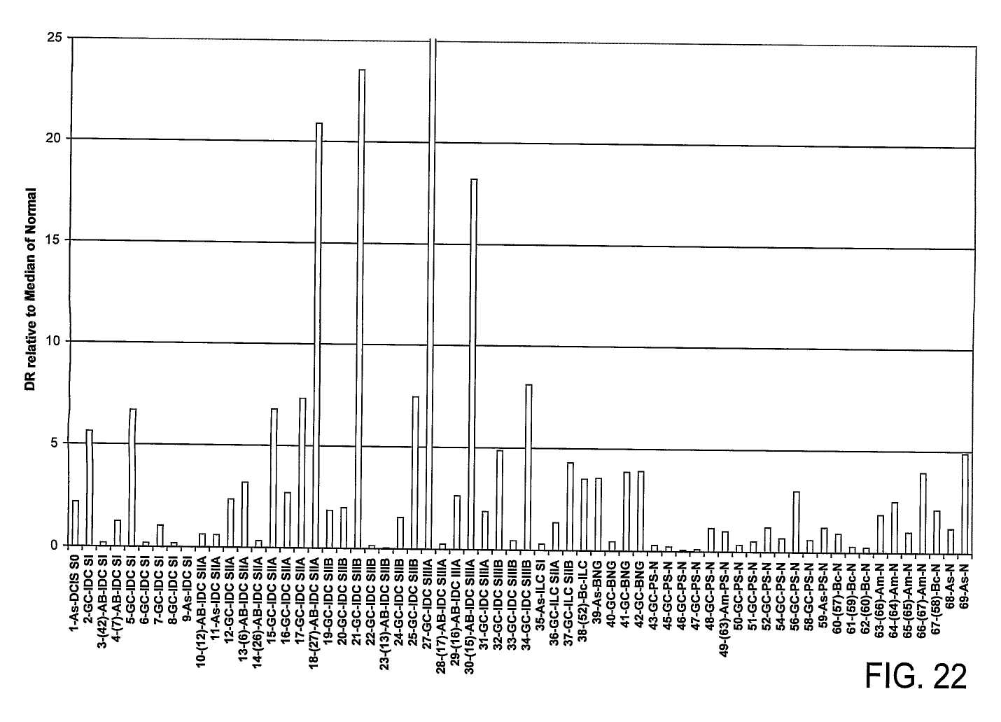

FIG. 22 is a histogram showing down regulation of the above-indicated Homo sapiens myeloma overexpressed gene transcripts in cancerous Breast samples relative to the normal samples.

As is evident from FIG. 22, the expression of Homo sapiens myeloma overexpressed gene transcripts detectable by the above amplicon in cancer samples was lower than in the non-cancerous samples (sample numbers 43, 45, 46, 47, 48, 49, 50, 51, 52, 54, 56, 58, 59, 60, 61, 62, 63, 64, 65, 66, 67, 68 and 69, Table 1_{—}7 above). Notably down regulation of at least 5 fold was found in 10 out of 37 adenocarcinoma samples.

Statistical analysis was applied to verify the significance of these results, as described below.

The P value for the difference in the expression levels of Homo sapiens myeloma overexpressed gene (in a subset of t(11;14) positive multiple myelomas) (MYEOV) transcripts detectable by the above amplicon in breast cancer samples versus the normal tissue samples was determined by T test as 9.90e-03.

Threshold of 5 fold over expression was found to differentiate between cancer and normal samples with P value of 4.62e-03 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: AA583399_seg17F (SEQ ID NO: 365) forward primer; and AA583399_seg17R (SEQ ID NO: 366) reverse primer.

The present invention also preferably encompasses any amplicon obtained through the use of any suitable primer pair; for example, for the above experiment, the following amplicon was obtained as a non-limiting illustrative example only of a suitable amplicon: AA583399_seg17 (SEQ ID NO: 367).

### Description for Cluster H13410

Cluster H13410 features 6 transcript(s) and 40 segment(s) of interest, the names for which are given in Tables 58 and 59, respectively, the sequences themselves are given in the “Sequence Listing” of the application. The selected protein variants are given in table 60.

TABLE 58
Transcripts of interest
Transcript Name

H13410_T8 (SEQ ID NO: 87)
H13410_T12 (SEQ ID NO: 88)
H13410_T18 (SEQ ID NO: 89)
H13410_T21 (SEQ ID NO: 90)
H13410_T27 (SEQ ID NO: 91)
H13410_T35 (SEQ ID NO: 92)

TABLE 59
Segments of interest
Segment Name

H13410_N6 (SEQ ID NO: 93)
H13410_N8 (SEQ ID NO: 94)
H13410_N10 (SEQ ID NO: 95)
H13410_N14 (SEQ ID NO: 96)
H13410_N20 (SEQ ID NO: 97)
H13410_N27 (SEQ ID NO: 98)
H13410_N29 (SEQ ID NO: 99)
H13410_N36 (SEQ ID NO: 100)
H13410_N37 (SEQ ID NO: 101)
H13410_N39 (SEQ ID NO: 102)
H13410_N42 (SEQ ID NO: 103)
H13410_N54 (SEQ ID NO: 104)
H13410_N56 (SEQ ID NO: 105)
H13410_N58 (SEQ ID NO: 106)
H13410_N68 (SEQ ID NO: 107)
H13410_N0 (SEQ ID NO: 108)
H13410_N1 (SEQ ID NO: 109)
H13410_N4 (SEQ ID NO: 110)
H13410_N5 (SEQ ID NO: 111)
H13410_N12 (SEQ ID NO: 112)
H13410_N16 (SEQ ID NO: 113)
H13410_N18 (SEQ ID NO: 114)
H13410_N22 (SEQ ID NO: 115)
H13410_N31 (SEQ ID NO: 116)
H13410_N33 (SEQ ID NO: 117)
H13410_N35 (SEQ ID NO: 118)
H13410_N44 (SEQ ID NO: 119)
H13410_N45 (SEQ ID NO: 120)
H13410_N46 (SEQ ID NO: 121)
H13410_N48 (SEQ ID NO: 122)
H13410_N49 (SEQ ID NO: 123)
H13410_N51 (SEQ ID NO: 124)
H13410_N55 (SEQ ID NO: 125)
H13410_N57 (SEQ ID NO: 126)
H13410_N59 (SEQ ID NO: 127)
H13410_N60 (SEQ ID NO: 128)
H13410_N67 (SEQ ID NO: 129)
H13410_N69 (SEQ ID NO: 130)
H13410_N70 (SEQ ID NO: 131)
H13410_N71 (SEQ ID NO: 132)
H13410_N10 (SEQ ID NO: 95)

TABLE 60
Proteins of interest

| Protein Name | Corresponding Transcript(s) |
|---|---|
| H13410_P4 (SEQ ID NO: 139) | H13410_T12 (SEQ ID NO: 88) |
| H13410_P8 (SEQ ID NO: 140) | H13410_T27 (SEQ ID NO: 91) |
| H13410_P9 (SEQ ID NO: 141) | H13410_T18 (SEQ ID NO: 89) |
| H13410_P20 (SEQ ID NO: 142) | H13410_T35 (SEQ ID NO: 92) |
| H13410_P46 (SEQ ID NO: 143) | H13410_T8 (SEQ ID NO: 87) |
| H13410_P47 (SEQ ID NO: 144) | H13410_T21 (SEQ ID NO: 90) |

These sequences are variants of the known protein maba1 (SwissProt accession identifier NP_{—}065826 (SEQ ID NO:344)), referred to herein as the previously known protein.

Additional sequences for known maba1 proteins are given in SEQ ID NO:133-138. The sequences of known maba1 mRNA transcript is given in SEQ ID NO:491. The sequence given in SEQ ID NO: 420 represents the segment corresponding to H13410_node10, from which the amplicon, used for detection of differential expression of the known maba1 transcript, derives.

The variants H13410_P4 and H13410_P9 were also previously disclosed by the inventors in published PCT application no WO2005/071058, hereby incorporated by reference as if fully set forth herein, but have now been shown to have novel and surprising diagnostic uses as described herein for other variants of cluster H13410.

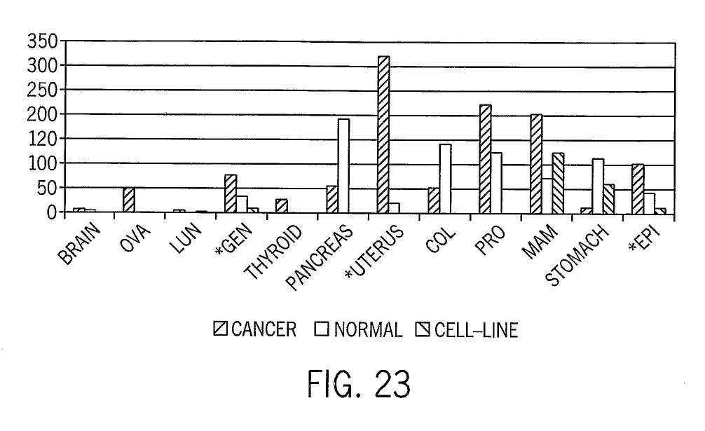

Cluster H13410 can be used as a diagnostic marker according to overexpression of transcripts of this cluster in cancer. Expression of such transcripts in normal tissues is also given according to the previously described methods. The term “number” in the left hand column of the table and the numbers on the y-axis of FIG. 23 refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 23 and Table 61. This cluster is overexpressed (at least at a minimum level) in the following pathological conditions: a mixture of malignant tumors from different tissues, uterine malignancies and epithelial malignant tumors.

TABLE 61
Normal tissue distribution

| Name of Tissue | Number |
|---|---|
| brain | 7 |
| ovary | 0 |
| lung | 0 |
| general | 34 |
| Thyroid | 0 |
| pancreas | 194 |
| uterus | 22 |
| colon | 144 |
| prostate | 127 |
| breast | 73 |
| stomach | 115 |
| epithelial | 47 |

TABLE 62
P values and ratios for expression in cancerous tissue

| Name of Tissue | P1 | P2 | SP1 | R3 | SP2 | R4 |
|---|---|---|---|---|---|---|
| brain | 8.2e−01 | 8.9e−01 | 5.1e−01 | 1.2 | 7.5e−01 | 0.7 |
| ovary | 3.8e−01 | 4.2e−01 | 3.2e−01 | 2.4 | 4.5e−01 | 1.9 |
| lung | 1.1e−01 | 1.4e−01 | N/A | N/A | N/A | N/A |
| general | 1.7e−04 | 5.5e−02 | 2.1e−07 | 1.9 | 4.9e−02 | 1.1 |
| Thyroid | 3.6e−01 | 3.6e−01 | N/A | N/A | N/A | N/A |
| pancreas | 5.4e−01 | 6.7e−01 | 1.0e+00 | 0.3 | 1.0e+00 | 0.2 |
| uterus | 3.1e−02 | 2.2e−01 | 9.8e−05 | 3.8 | 1.2e−02 | 2.1 |
| colon | 7.0e−01 | 7.9e−01 | 9.4e−01 | 0.5 | 9.8e−01 | 0.4 |
| prostate | 1.3e−01 | 2.9e−01 | 1.1e−01 | 1.5 | 3.8e−01 | 1.0 |
| breast | 1.8e−01 | 3.2e−01 | 9.6e−02 | 2.0 | 1.4e−01 | 1.4 |
| stomach | 4.5e−01 | 8.1e−01 | 1.0e+00 | 0.3 | 9.4e−01 | 0.4 |
| epithelial | 5.6e−03 | 2.1e−01 | 1.1e−04 | 1.7 | 1.5e−01 | 1.0 |

As noted above, cluster H13410 features 6 transcript(s), which were listed in Table 58 above. These transcript(s) encode for protein(s) which are variant(s) of protein maba1 (SEQ ID NO: 133). A description of each variant protein according to the present invention is now provided.

Variant protein H13410_P4 (SEQ ID NO: 139) according to the present invention has an amino acid sequence as given in the “Sequence Listing” of the application; it is encoded by transcript(s) H13410_T12 (SEQ ID NO: 88). An alignment is given to the known protein (maba1, SEQ ID NOs:133-138) in the alignment table, on the attached CDROM. One or more alignments to one or more previously published protein sequences are given in the alignment table, on the attached CDROM. A brief description of the relationship of the variant protein according to the present invention to each such aligned protein is as follows:

### 1. Comparison Report Between H13410_P4 (SEQ ID NO: 139) and Q6UXG2_HUMAN (SEQ ID NO: 137) (or NP_{—}065826 (SEQ ID NO:344)):

A. An isolated chimeric polypeptide encoding for H13410_P4 (SEQ ID NO: 139), comprising a first amino acid sequence being at least about 90% homologous to MAEPGHSHHLSARVRGRTERRIPRLWRLLLWAGTAFQVTQGTGPELHACKESEYHYEYTACDSTGSRWR VAVPHTPGLCTSLPDPVKGTECSFSCNAGEFLDMKDQSCKPCAEGRYSLGTGIRFDEWDELPHGFASLSAN MELDDSAAESTGNCTSSKWVPRGDYIASNTDECTATLMYAVNLKQSGTVNFEYYYPDSSIIFEFFVQNDQ CQPNADDSRWMKTTEKGWEFHSVELNRGNNVLYWRTTAFSVWTKVPKPVLVRNIAITGVAYTSECFPCK PGTYADKQGSSFCKLCPANSYSNKGETSCHQCDPDKYSEKGSSSCNVRPACTDKDYFYTHTACDANGET QLMYKWAKPKICSEDLEGAVKLPASGVKTHCPPCNPGFFKTNNSTCQPCPYGSYSNGSDCTRCPAGTEPA VGFEYKWWNTLP NMETTVLSGINFEYKGMTGWEVAGDHIYTAAGASDNDFMILTLVVPGFRPPQSVMA DTENKEVARITFVFETLCSVNCELYFMV corresponding to amino acids 1-515 of Q6UXG2_HUMAN (SEQ ID NO: 137), which also corresponds to amino acids 1-515 of H13410_P4 (SEQ ID NO: 139), and a second amino acid sequence being at least about 70%, optionally at least 80%, preferably at least 85%, more preferably at least 90% and most preferably at least 95% homologous to a polypeptide having the sequence VRFPFFAR (SEQ ID NO: 499) corresponding to amino acids 516-523 of H13410_P4 (SEQ ID NO: 139), wherein said first amino acid sequence and second amino acid sequence are contiguous and in a sequential order.

B. An isolated polypeptide encoding for an edge portion of H13410_P4 (SEQ ID NO: 139), comprising an amino acid sequence being at least about 70%, optionally at least about 80%, preferably at least about 85%, more preferably at least about 90% and most preferably at least about 95% homologous to the sequence VRFPFFAR (SEQ ID NO: 499) of H13410_P4 (SEQ ID NO: 139).

The localization of the variant protein was determined according to results from a number of different software programs and analyses, including analyses from SignalP and other specialized programs. The variant protein is believed to be secreted.

Variant protein H13410_P4 (SEQ ID NO: 139) also has the following non-silent SNPs (Single Nucleotide Polymorphisms) as listed in Table 63, (given according to their position(s) on the amino acid sequence, with the alternative amino acid(s) listed).

TABLE 63
Amino acid mutations

| SNP position(s) on amino acid | |
|---|---|
| sequence | Alternative amino acid(s) |
| 16 | G -> * |
| 16 | G -> R |
| 86 | V -> I |

Variant protein H13410_P4 (SEQ ID NO: 139) is encoded by the following transcript(s): H13410_T12 (SEQ ID NO: 88), for which the coding portion starts at position 138 and ends at position 1706. The transcript also has the following SNPs as listed in Table 64 (given according to their position on the nucleotide sequence, with the alternative nucleic acid listed).

TABLE 64
Nucleic acid SNPs

| | SNP position(s) on nucleotide |
|---|---|
| Polymorphism | sequence |
| G -> A | 183, 393 |
| G -> T | 183 |
| T -> C | 278, 2468, 2359 |
| C -> T | 2140, 3417, 2428 |
| C -> A | 2258 |
| A -> G | 2386, 2902 |
| C -> G | 3271 |
| C -> | 3271 |

Variant protein H13410_P8 (SEQ ID NO: 140) according to the present invention has an amino acid sequence as given in the “Sequence Listing” of the application; it is encoded by transcript(s) H13410_T27 (SEQ ID NO: 91). An alignment is given to the known protein (maba1 SEQ ID NOs:133-138) in the alignment table, on the attached CDROM. One or more alignments to one or more previously published protein sequences are given in the alignment table, on the attached CDROM. A brief description of the relationship of the variant protein according to the present invention to each such aligned protein is as follows:

### 1. Comparison Report Between H13410_P8 (SEQ ID NO: 140) and Q6UXG2_HUMAN (SEQ ID NO: 137):

A. An isolated chimeric polypeptide encoding for H13410_P8 (SEQ ID NO: 140), comprising a first amino acid sequence being at least about 90% homologous to MAEPGHSHHLSARVRGRTERRIPRLWRLLLWAGTAFQVTQGTGPELHACKESEYHYEYTACDSTGSRWR VAVPHTPGLCTSLPDPVKGTECSFSCNAGEFLDMKDQSCKPCAEGRYSLGTGIRFDEWDELPHGFASLSAN MELDDSAAESTGNCTSSKWVPRGDYIASNTDECTATLMYAVNLKQSGTVNFEYYYPDSSIIFEFFVQNDQ CQPNADDSRWMKTTEKGWEFHSVELNRGNNVLYWRTTAFSVWTKVPKPVLVRNIAITGVAYTSECFPCK PGTYADKQGSSFCKLCPANSYSNKGETSCHQCDPDKYSEKGSSSCNVRPACTDKDYFYTHTACDANGET QLMYKWAKPKICSEDLEGAVKLPASGVKTHCPPCNPGFFKTNNSTCQPCPYGSYSNGSDCTRCPAGTEPA VGFEYKWWNTLPTNMETTVLSGINFEYKGMTGWEVAGDHIYTAAGASDNDFMILTLVVPGFRPPQSVMA DTENKEVARITFVFETLCSVNCELYFMVGVNSRTNTPVETWKGSKGKQSYTYIIEENTTTSFTWAFQRTTF HEASRKYTNDVAKIYSINVTNVMNGVASYCRPCALEASDVGSSCTSCPAGYYIDRDSGTCHSCPPNTILKA HQPYGVQACVPCGPGTKNNKIHSLCYNDCTFSRNTPIRTFNYNFSALANTVTLAGGPSFTSKGLKYFHHFT LSLCGNQGRKMSVCTDNVTDLRIPEGESGFSKSITAYVCQAVIIPPEVTGYKAGVSSQPVSLADRLIGVTTD MTLDGITSPAELFHLESLGIPDVIFFYRSNDVTQSCSSGRSTTIRVRCSPQKTVPGSLLLPGTCSDGTCDGCN FHFLWESAAACPLCSVADYHAIVSSCVAGIQ corresponding to amino acids 1-876 of Q6UXG2_HUMAN (SEQ ID NO: 137), which also corresponds to amino acids 1-876 of H13410_P8 (SEQ ID NO: 140), and a second amino acid sequence being at least about 70%, optionally at least 80%, preferably at least 85%, more preferably at least 90% and most preferably at least 95% homologous to a polypeptide having the sequence VGFPLIGHFHEIRREWEI (SEQ ID NO: 500) corresponding to amino acids 877-893 of H13410_P8 (SEQ ID NO: 140), wherein said first amino acid sequence and second amino acid sequence are contiguous and in a sequential order.

B. An isolated polypeptide encoding for an edge portion of H13410_P8 (SEQ ID NO: 140), comprising an amino acid sequence being at least about 70%, optionally at least about 80%, preferably at least about 85%, more preferably at least about 90% and most preferably at least about 95% homologous to the sequence VGFPLIGHFHERREWEI (SEQ ID NO: 500) of H13410_P8 (SEQ ID NO: 140).

### 2. Comparison Report Between H13410_P8 (SEQ ID NO: 140) and NP_{—}065826 (SEQ ID NO:344):

A. An isolated chimeric polypeptide encoding for H13410_P8 (SEQ ID NO: 140), comprising a first amino acid sequence being at least about 90% homologous to MAEPGHSHHLSARVRGRTERRIPRLWRLLLWAGTAFQVTQGTGPELHACKESEYHYEYTACDSTGSRWR VAVPHTPGLCTSLPDPVKGTECSFSCNAGEFLDMKDQSCKPCAEGRYSLGTGIRFDEWDELPHGFASLSAN MELDDSAAESTGNCTSSKWVPRGDYIASNTDECTATLMYAVNLKQSGTVNFEYYYPDSSIIFEFFVQNDQ CQPNADDSRWMKTTEKGWEFHSVELNRGNNVLYWRTTAFSVWTKVPKPVLVRNIAITGVAYTSECFPCK PGTYADKQGSSFCKLCPANSYSNKGETSCHQCDPDKYSEKGSSSCNVRPACTDKDYFYTHTACDANGET QLMYKWAKPKICSEDLEGAVKLPASGVKTHCPPCNPGFFKTNNSTCQPCPYGSYSNGSDCTRCPAGTEPA VGFEYKWWNTLPTNMETTVLSGINFEYKGMTGWEVAGDHIYTAAGASDNDFMILTLVVPGFRPPQSVMA DTENKEVARITFVFETLCSVNCELYFMVGVNSRTNTPVETWKGSKGKQSYTYIIEENTTTSFTWAFQRTTF HEASRKYTNDVAKIYSINVTNVMNGVASYCRPCALEASDVGSSCTSCPAGYYIDRDSGTCHSCP corresponding to amino acids 1-622 of NP_{—}065826 (SEQ ID NO:344), which also corresponds to amino acids 1-622 of H13410_P8 (SEQ ID NO: 140), a bridging amino acid P corresponding to amino acid 623 of H13410_P8 (SEQ ID NO: 140), a second amino acid sequence being at least about 90% homologous to NTILKAHQPYGVQACVPCGPGTKNNKIHSLCYNDCTFSRNTPTRTFNYNFSALANTVTLAGGPSFTSKGLK YFHHFTLSLCGNQGRKMSVCTDNVTDLRIPEGESGFSKSITAYVCQAVIIPPEVTGYKAGVSSQPVSLADRL IGVTTDMTLDGITSPAELFHLESLGIPDVIFFYRSNDVTQSCSSGRSTTIRVRCSPQKTVPGSLLLPGTCSDGT CDGCNFHFLWESAAACPLCSVADYHAIVSSCVAGIQ corresponding to amino acids 624-876 of NP_{—}065826 (SEQ ID NO:344), which also corresponds to amino acids 624-876 of H13410_P8 (SEQ ID NO: 140), and a third amino acid sequence being at least about 70%, optionally at least 80%, preferably at least 85%, more preferably at least 90% and most preferably at least 95% homologous to a polypeptide having the sequence VGFPLIGHFHERREWEI (SEQ ID NO: 500) corresponding to amino acids 877-893 of H13410_P8 (SEQ ID NO: 140), wherein said first amino acid sequence, bridging amino acid, second amino acid sequence and third amino acid sequence are contiguous and in a sequential order.

### 3. Comparison Report Between H13410_P8 (SEQ ID NO: 140) and Q8N1G3_HUMAN (SEQ ID NO: 136):

A. An isolated chimeric polypeptide encoding for H13410_P8 (SEQ ID NO: 140), comprising a first amino acid sequence being at least about 70%, optionally at least 80%, preferably at least 85%, more preferably at least 90% and most preferably at least 95%, homologous to a polypeptide having the sequence MAEPGHSHHLSARVRGRTERRIPRLWRLLLWAGTAFQVTQGTGPELHACKESEYHYEYTACDSTGSRWR VAVPHTPGLCTSLPDPVKGTECSFSCNAGEFLDMKDQSCKPCAEGRYSLGTGIRFDEWDELPHGFASLSAN MELDDSAAESTGNCTSSKWVPRGDYIASNTDECTATLMYAVNLKQSGTVNFEYYYPDSSIIFEFFVQNDQ CQPNADDSRWMKTTEKGWEFHSVELNRGNNVLYWRTTAFSVWTKVPKPVLVRNIAITGVAYTSECFPCK PGTYADKQGSSFCKLCPANSYSNKGETSCHQCDPDKYSEKGSSSCNVRPACTDKDYFYTHTACDANGET QL (SEQ ID NO: 501) corresponding to amino acids 1-350 of H13410_P8 (SEQ ID NO: 140), a second amino acid sequence being at least about 90% homologous to MYKWAKPKICSEDLEGAVKLPASGVKTHCPPCNPGFFKTNNSTCQPCPYGSYSNGSDCTRCPAGTEPAVG FEYKWWNTLPTNMETTVLSGINFEYKGMTGWEVAGDHIYTAAGASDNDFMILTLVVPGFRPPQSVMADT ENKEVARITFVFETLCSVNCELYFMVGVNSRTNTPVETWKGSKGKQSYTYIIEENTTTSFTWAFQRTTFHE ASRKYTNDVAKIYSINVTNVMNGVASYCRPCALEASDVGSSCTSCPAGYYIDRDSGTCHSCPPNTILKAHQ PYGVQACVPCGPGTKNNKIHSLCYNDCTFSRNTPTRTFNYNFSALANTVTLAGGPSFTSKGLKYFHHFTLS LCGNQGRKMSVCTDNVTDLRIPEGESGFSKSITAYVCQAVIIPPEVTGYKAGVSSQPVSLADRLIGVTTDM TLDGITSPAELFHLESLGIPDVIFFYRSNDVTQSCSSGRSTTIRVRCSPQKTVPGSLLLPGTCSDGTCDGCNFH FLWESAAACPLCSVADYHAIVSSCVAGIQ corresponding to amino acids 1-526 of Q8N1G3_HUMAN (SEQ ID NO: 136), which also corresponds to amino acids 351-876 of H13410_P8 (SEQ ID NO: 140), and a third amino acid sequence being at least about 70%, optionally at least 80%, preferably at least 85%, more preferably at least 90% and most preferably at least 95% homologous to a polypeptide having the sequence VGFPLIGHFHERREWEI (SEQ ID NO: 500) corresponding to amino acids 877-893 of H13410_P8 (SEQ ID NO: 140), wherein said first amino acid sequence, second amino acid sequence and third amino acid sequence are contiguous and in a sequential order.

B. An isolated polypeptide encoding for a head of H13410_P8 (SEQ ID NO: 140), comprising a polypeptide being at least 70%, optionally at least about 80%, preferably at least about 85%, more preferably at least about 90% and most preferably at least about 95% homologous to the sequence

(SEQ ID NO: 501)

MAEPGHSHHLSARVRGRTERRIPRLWRLLLWAGTAFQVTQGTGPELHACK
ESEYHYEYTACDSTGSRWRVAVPHTPGLCTSLPDPVKGTECSFSCNAGEF
LDMKDQSCKPCAEGRYSLGTGIRFDEWDELPHGFASLSANMELDDSAAES
TGNCTSSKWVPRGDYIASNTDECTATLMYAVNLKQSGTVFEYYYPDSSII
FEFFVQNDQCQPNADDSRWMKTTEKGWEFHSVELNRGNNVLYWRTTAFSV
WTKVPKPVLVRNIAITGVAYTSECFPCKPGTYADKQGSSFCKLCPANSYS
NKGETSCHQCDPDKYSEKGSSSCNVRPACTDKDYFYTHTACDANGETQL
of

(SEQ ID NO: 140)

H13410_P8.

### 4. Comparison Report Between H13410_P8 (SEQ ID NO: 140) and Q5T5C9_HUMAN (SEQ ID NO: 138):

A. An isolated chimeric polypeptide encoding for H13410_P8 (SEQ ID NO: 140), comprising a first amino acid sequence being at least about 70%, optionally at least 80%, preferably at least 85%, more preferably at least 90% and most preferably at least 95%, homologous to a polypeptide having the sequence MAEPGHSHHLSARVRGRTERRIPRLWRLLLWAGTAFQVTQGTGPELHACKESEYHYEYTACDSTGSRWR VAVPHTPGLCTSLPDPVKGTECSFSCNAGEFLDMKDQSCKPCAEGRYSLGTGIRFDEWDELPHGFASLSAN MELDDSAAESTGNCTSSKWVPRGDYIASNTDECTATLMYAVNLKQSGTVNFEYYYPDSSIIFEFFVQNDQ CQPNADDSRWMKTTEKGWEFHSVELNRGNNVLYWRTTAFSVWTKVPKPVLVRNIAITGVAYTSECFPCK PGTYADKQGSSFCKLCPANSYSNKGETSCHQCDPDKYSEKGSSSCNVRPACTDKDYFYTHTACDANGET QL (SEQ ID NO: 501) corresponding to amino acids 1-350 of H13410_P8 (SEQ ID NO: 140), a second amino acid sequence being at least about 90% homologous to MYKWAKPKICSEDLEGAVKLPASGVKTHCPPCNPGFFKTNNSTCQPCPYGSYSNGSDCTRCPAGTEPAVG FEYKWUVNTLPTNMETTVLSGINFEYKGMTGWEVAGDHIYTAAGASDNDFMILTLVVPGFRPPQSVMADT ENKEVARITFVFETLCSVNCELYFMVGVNSRTNTPVETWKGSKGKQSYTYIIEENTTTSFTWAFQRTTFHE ASRKYTNDVAKIYSINVTNVMNGVASYCRPCALEASDVGSSCTSCPAGYYIDRDSGTCHSCP corresponding to amino acids 1-272 of Q5T5C9-HUMAN (SEQ ID NO: 138), which also corresponds to amino acids 351-622 of H13410_P8 (SEQ ID NO: 140), a bridging amino acid P corresponding to amino acid 623 of H13410_P8 (SEQ ID NO: 140), a third amino acid sequence being at least about 90% homologous to NTILKAHQPYGVQACVPCGPGTKNNKIHSLCYNDCTFSRNTPTRTFNYNFSALANTVTLAGGPSFTSKGLK YFHHFTLSLCGNQGRKMSVCTDNVTDLRIPEGESGFSKSITAYVCQAVIIPPEVTGYKAGVSSQPVSLADRL IGVTTDMTLDGITSPAELFHLESLGIPDVIFFYRSNDVTQSCSSGRSTTIRVRCSPQKTVPGSLLLPGTCSDGT CDGCNFHFLWESAAACPLCSVADYHAIVSSCVAGIQ corresponding to amino acids 274-526 of Q5T5C9_HUMAN (SEQ ID NO: 138), which also corresponds to amino acids 624-876 of H13410_P8 (SEQ ID NO: 140), and a fourth amino acid sequence being at least about 70%, optionally at least 80%, preferably at least 85%, more preferably at least 90% and most preferably at least 95% homologous to a polypeptide having the sequence VGFPLIGHFHERREWEI (SEQ ID NO: 500) corresponding to amino acids 877-893 of H13410_P8 (SEQ ID NO: 140), wherein said first amino acid sequence, second amino acid sequence, bridging amino acid, third amino acid sequence and fourth amino acid sequence are contiguous and in a sequential order.

### 5. Comparison Report Between H13410_P8 (SEQ ID NO: 140) and Q9P2M2_HUMAN (SEQ ID NO: 135):

A. An isolated chimeric polypeptide encoding for H13410_P8 (SEQ ID NO: 140), comprising a first amino acid sequence being at least about 70%, optionally at least 80%, preferably at least 85%, more preferably at least 90% and most preferably at least 95%, homologous to a polypeptide having the sequence MAEPGHSHHLSARVRGRTERRIPRLWRLLLWAGTAFQVTQGTGPELHACKESEYHYEYTACDSTGSRWR VAVPHTPGLCTSLPDPVKGTECSFSCNAGEFLDMKDQSCKPCAEGRYSLGTGIRFDEWDELPHGFASLSAN MELDDSAAESTGNCTSSKWVPRGDYIASNTDECTATLMYAVNLKQSGTVNFEYYYPDSSIIFEFFVQNDQ CQPNADDSRWMKTTEKGWEFHSVELNRGNNVLYWRTTAFSVWTKVPKPVLVRNIAITGVAYTSECFPCK PGTYADKQGSSFCKLCPANSYSNKGETSCHQCDPDKYSEKGSSSCNVRPACTDKDYFYTHTACDANGET QLMYKWAKPKICSEDLEGAVKLPASGVKTHCPPCNPGFFKTNNSTCQPCPYGSYSNGSDCTRCPAGTEPA VGFEYK (SEQ ID NO: 502) corresponding to amino acids 1-424 of H13410_P8 (SEQ ID NO: 140), a second amino acid sequence being at least about 90% homologous to WWNTLPTNMETTVLSGINFEYKGMTGWEVAGDHIYTAAGASDNDFMILTLVVPGFRPPQSVMADTENKE VARITFVFETLCSVNCELYFMVGVNSRTNTPVETWKGSKGKQSYTYIIEENTTTSFTWAFQRTTFHEASRK YTNDVAKIYSINVTNVMNGVASYCRPCALEASDVGSSCTSCPAGYYIDRDSGTCHSCPPNTILKAHQPYGV QACVPCGPGTKNNKIHSLCYNDCTFSRNTPTRTFNYNFSALANTVTLAGGPSFTSKGLKYFHHFTLSLCGN QGRKMSVCTDNVTDLRIPEGESGFSKSITAYVCQAVIIPPEVTGYKAGVSSQPVSLADRLIGVTTDMTLDGI TSPAELFHLESLGIPDVIFFYRSNDVTQSCSSGRSTTIRVRCSPQKTVPGSLLLPGTCSDGTCDGCNFBFLWE SAAACPLCSVADYHAIVSSCVAGIQ corresponding to amino acids 1-452 of Q9P2M2_HUMAN (SEQ ID NO: 135), which also corresponds to amino acids 425-876 of H13410_P8 (SEQ ID NO: 140), and a third amino acid sequence being at least about 70%, optionally at least 80%, preferably at least 85%, more preferably at least 90% and most preferably at least 95% homologous to a polypeptide having the sequence VGFPLIGHFHERREWEI (SEQ ID NO: 500) corresponding to amino acids 877-893 of H13410_P8 (SEQ ID NO: 140), wherein said first amino acid sequence, second amino acid sequence and third amino acid sequence are contiguous and in a sequential order.

B. An isolated polypeptide encoding for a head of H13410_P8 (SEQ ID NO: 140), comprising a polypeptide being at least 70%, optionally at least about 80%, preferably at least about 85%, more preferably at least about 90% and most preferably at least about 95% homologous to the sequence

(SEQ ID NO: 502)

MAEPGHSHHLSARVRGRTERRIPRLWRLLLWAGTAFQVTQGTGPELHACK
ESEYHYEYTACDSTGSRWRVAVPHTPGLCTSLPDPVKGTECSFSCNAGEF
LDMKDQSCKPCAEGRYSLGTGIRFDEWDELPHGFASLSANMELDDSAAES
TGNCTSSKWVPRGDYIASNTDECTATLMYAVNLKQSGTVNFEYYYPDSSI
IFEFVQNDQCQPNADDSRWMKTTEKGWEFHSVELNRGNNVLYWRTTAFSV
WTKVPKPVLVRNIAITGVAYTSECFPCKPGTYADKQGSSFCKLCPANSYS
NKGETSCHQCDPDKYSEKGSSSCNVRPACTDKDYFYHTACDANGETQLMY
KWAKPKICSEDLEGAVKLPASGVKTHCPPCNPGFFKTNNSTCQPCPYGSY
SNGSDCTRCPAGTEPAVGFEYK
of

(SEQ ID NO: 140)

H13410_P8.

The localization of the variant protein was determined according to results from a number of different software programs and analyses, including analyses from SignalP and other specialized programs. The variant protein is believed to be secreted.

Variant protein H13410_P8 (SEQ ID NO: 140) also has the following non-silent SNPs (Single Nucleotide Polymorphisms) as listed in Table 65, (given according to their position(s) on the amino acid sequence, with the alternative amino acid(s) listed).

TABLE 65
Amino acid mutations

| SNP position(s) on amino acid | |
|---|---|
| sequence | Alternative amino acid(s) |
| 16 | G -> * |
| 16 | G -> R |
| 86 | V -> I |
| 623 | P -> T |

Variant protein H13410_P8 (SEQ ID NO: 140) is encoded by the following transcript(s): H13410_T27 (SEQ ID NO: 91), for which the coding portion starts at position 138 and ends at position 2816. The transcript also has the following SNPs as listed in Table 66 (given according to their position on the nucleotide sequence, with the alternative nucleic acid listed; H13410_P8 (SEQ ID NO: 140)).

TABLE 66
Nucleic acid SNPs

| | SNP position(s) on nucleotide |
|---|---|
| Polymorphism | sequence |
| G -> A | 183, 393 |
| G -> T | 183 |
| T -> C | 278, 2105, 2214 |
| C -> T | 1886, 2174, 3738 |
| C -> A | 2004 |
| A -> G | 2132, 2648 |
| C -> G | 3592 |
| C -> | 3592 |

Variant protein H13410_P9 (SEQ ID NO: 141) according to the present invention has an amino acid sequence as given in the “Sequence Listing” of the application; it is encoded by transcript(s) H13410_T18 (SEQ ID NO: 89). An alignment is given to the known protein (maba1 SEQ ID NOs:133-138) in the alignment table, on the attached CDROM. One or more alignments to one or more previously published protein sequences are given in the alignment table, on the attached CDROM. A brief description of the relationship of the variant protein according to the present invention to each such aligned protein is as follows:

### 1. Comparison Report Between H13410_P9 (SEQ ID NO: 141) and Q6UXG2_HUMAN (SEQ ID NO: 137):

A. An isolated chimeric polypeptide encoding for H13410_P9 (SEQ ID NO: 141), comprising a first amino acid sequence being at least about 90% homologous to MAEPGHSHHLSARVRGRTERRIPRLWRLLLWAGTAFQVTQGTGPELHACKESEYHYEYTACDSTGSRWR VAVPHTPGLCTSLPDPVKGTECSFSCNAGEFLDMKDQSCKPCAEGRYSLGTGIRFDEWDELPHGFASLSAN MELDDSAAESTGNCTSSKWVPRGDYIASNTDECTATLMYAVNLKQSGTVNFEYYYPDSSRIFEFFVQNDQ CQPNADDSRWMKTTEKGWEFHSVELNRGNNVLYWRTTAFSVWTKVPKPVLVRNIAITGVAYTSECFPCK PGTYADKQGSSFCKLCPANSYSNKGETSCHQCDPDKYSEKGSSSCNVRPACTDKDYFYTHTACDANGET QLMYKWAKPKICSEDLEGAVKLPASGVKTHCPPCNPGFFKTNNSTCQPCPYGSYSNGSDCTRCPAGTEPA VGFEYKWWNTLPTNMETTVLSGINFEYKGMTGWEVAGDHIYTAAGASDNDFMILTLVVPGFRPPQSVMA DTENKEVARITFVFETLCSVNCELYFMVGVNSRTNTPVETWKGSKGKQSYTYIIEENTTTSFTWAFQRTTF BEASRKYTNDVAKIYSINVTNVMNGVASYCRPCALEASDVGSSCTSCPAGYYIDRDSGTCHSCPPNTILKA HQPYGVQACVPCGPGTKNNKIHSLCYNDCTFSRNTPTRTFNYNFSALANTVTLAGGPSFTSKGLKYFHHFT LSLCGNQGRKMSVCTDNVTDLRIPEGESGFSKSITAYVCQAVIIPPEVTGYKAGVSSQPVSLADRLIGVTTD MTLDGITSPAELFHLESLGIPDVIFFYRSNDVTQSCSSGRSTTIRVRCSPQKTVPGSLLLPGTCSDGTCDGCN FHFLWESAAACPLCSVADYHAIVSSCVAGIQKTTYVWREPKLCSGGISLPEQRVTICKTIDFWLKVGISAGT CTAILLTVLTCYFWKKNQK corresponding to amino acids 1-936 of Q6UXG2_HUMAN (SEQ ID NO: 137), which also corresponds to amino acids 1-936 of H13410_P9 (SEQ ID NO: 141), and a second amino acid sequence being at least about 70%, optionally at least 80%, preferably at least 85%, more preferably at least 90% and most preferably at least 95% homologous to a polypeptide having the sequence YMLRYWSVGMDRVGLLIRENI (SEQ ID NO: 503) corresponding to amino acids 937-957 of H13410_P9 (SEQ ID NO: 141), wherein said first amino acid sequence and second amino acid sequence are contiguous and in a sequential order.

B. An isolated polypeptide encoding for an edge portion of H13410_P9 (SEQ ID NO: 141), comprising an amino acid sequence being at least about 70%, optionally at least about 80%, preferably at least about 85%, more preferably at least about 90% and most preferably at least about 95% homologous to the sequence YMLRYWSVGMDRVGLLIRENI (SEQ ID NO: 503) of H13410_P9 (SEQ ID NO: 141).

### 2. Comparison Report Between H13410_P9 (SEQ ID NO: 141) and NP_{—}065826 (SEQ ID NO:344):

A. An isolated chimeric polypeptide encoding for H13410_P9 (SEQ ID NO: 141), comprising a first amino acid sequence being at least about 90% homologous to

MAEPGHSHHLSARVRGRTERRIPRLWRLLLWAGTAFQVTQGTGPELHACK
ESEYHYEYTACDSTGSRWRVAVPHTPGLCTSLPDPVKGTECSFSCNAGEF
LDMKDQSCKPCAEGRYSLGTGIRFDEWDELPHGFASLSANMELDDSAAES
TGNCTSSKWVPRGDYIASNTDECTATLMYAVNLKQSGTVNFEYYYPDSSI
IFEFFVQNDQCQPNADDSRWMKTTEKGWEFHSVELNRGNNVLYWRTTAFS
VWTKVPKPVLVRNIAITGVAYTSECFPCKPGTYADKQGSSFCKLCPANSY
SNKGETSCHQCDPDKYSEKGSSSCNVRPACTDKDYFYTHTACDANGETQL
MYKWAKPKICSEDLEGAVKLPASGVKTHCPPCNPGFFKTNNSTCQPCPYG
SYSNGSDCTRCPAGTEPAVGFEYKWWNTLPTNMETTVLSGINFEYKGMTG
WEVAGDHIYTAAGASDNDFMILTLVVPGFRPPQSVMADTENKEVARITFV
FETLCSVNCELYFMVGVNSRTNTPVETWKGSKGKQSYTYIIEENTTTSFT
WAFQRTTFHEASRKYTNDVAKIYSINVTNVMNGVASYCRPCALEASDVGS
SCTSCPAGYYIDRDSGTCHSCP

corresponding to amino acids 1-622 of NP_{—}065826 (SEQ ID NO:344), which also corresponds to amino acids 1-622 of H13410_P9 (SEQ ID NO: 141), a bridging amino acid P corresponding to amino acid 623 of H13410_P9 (SEQ ID NO: 141), a second amino acid sequence being at least about 90% homologous to NTILKAHQPYGVQACVPCGPGTKNNKIHSLCYNDCTFSRNTPTRTFNYNFSALANTVTLAGGPSFTSKGLK YFHHFTLSLCGNQGRKMSVCTDNVTDLRIPEGESGFSKSITAYVCQAVIIPPEVTGYKAGVSSQPVSLADRL IGVTTDMTLDGITSPAELFHLESLGIPDVIFFYRSNDVTQSCSSGRSTTIRVRCSPQKTVPGSLLLPGTCSDGT CDGCNFHFLWESAAACPLCSVADYHAIVSSCVAGIQKTTYVWREPKLCSGGISLPEQRVTICKTIDFWLKV GISAGTCTAILLTVLTCYFWKKNQK corresponding to amino acids 624-936 of NP_{—}065826 (SEQ ID NO:344), which also corresponds to amino acids 624-936 of H13410_P9 (SEQ ID NO: 141), and a third amino acid sequence being at least about 70%, optionally at least 80%, preferably at least 85%, more preferably at least 90% and most preferably at least 95% homologous to a polypeptide having the sequence YMLRYWSVGMDRVGLLIRENI (SEQ ID NO: 503) corresponding to amino acids 937-957 of H13410_P9 (SEQ ID NO: 141), wherein said first amino acid sequence, bridging amino acid, second amino acid sequence and third amino acid sequence are contiguous and in a sequential order.

### 3. Comparison Report Between H13410_P9 (SEQ ID NO: 141) and Q8N1G3_HUMAN (SEQ ID NO: 136):

A. An isolated chimeric polypeptide encoding for H13410_P9 (SEQ ID NO: 141), comprising a first amino acid sequence being at least about 70%, optionally at least 80%, preferably at least 85%, more preferably at least 90% and most preferably at least 95%, homologous to a polypeptide having the sequence MAEPGHSHHLSARVRGRTERRIPRLWRLLLWAGTAFQVTQGTGPELHACKESEYHYEYTACDSTGSRWR VAVPHTPGLCTSLPDPVKGTECSFSCNAGEFLDMKDQSCKPCAEGRYSLGTGIRFDEWDELPHGFASLSAN MELDDSAAESTGNCTSSKWVPRGDYIASNTDECTATLMYAVNLKQSGTVNFEYYYPDSSIIFEFFVQNDQ CQPNADDSRWMKTTEKGWEFHSVELNRGNNVLYWRTTAFSVWTKVPKPVLVRNIAITGVAYTSECFPCK PGTYADKQGSSFCKLCPANSYSNKGETSCHQCDPDKYSEKGSSSCNVRPACTDKDYFYTHTACDANGET QL (SEQ ID NO: 501) corresponding to amino acids 1-350 of H13410_P9 (SEQ ID NO: 141), a second amino acid sequence being at least about 90% homologous to

(SEQ ID NO: 136)

MYKWAKPKICSEDLEGAVKLPASGVKTHCPPCNPGFFKTNNSTCQPCPYG
SYSNGSDCTRCPAGTEPAVGFEYKWWNTLPTNMETTVLSGINFEYKGMTG
WEVAGDHIYTAAGASDNDFMILTLVVPGFRPPQSVMADTENKEVARITFV
FETLCSVNCELYFMVGVNSRTNTPVETWKGSKGKQSYTYIIEENTTTSFT
WAFQRTTFHEASRKYTNDVAKIYSINVTNVMNGVASYCRPCALEASDVGS
SCTSCPAGYYIDRDSGTCHSCPPNTILKAHQPYGVQACVPCGPGTKNNKI
HSLCYNDCTFSRNTPTRTFNYNFSALANTVTLAGGPSFTSKGLKYFHHFT
LSLCGNQGRKMSVCTDNVTDLRIPEGESGFSKSITAYVCQAVIIPPEVTG
YKAGVSSQPVSLADRLIGVTTDMTLDGITSPAELFHLESLGIPDVIFFYR
SNDVTQSCSSGRSTTIRVRCSPQKTVPGSLLLPGTCSDGTCDGCNFHFLW
ESAAACPLCSVADYHAIVSSCVAGIQKTTYVWREPKLCSGGISLPEQRVT
ICKTIDFWLKVGISAGTCTAILLTVLTCYFWKKNQK
corresponding to amino acids 1-586 of
Q8N1G3_HUMAN,

which also corresponds to amino acids 351-936 of H13410_P9 (SEQ ID NO: 141), and a third amino acid sequence being at least about 70%, optionally at least 80%, preferably at least 85%, more preferably at least 90% and most preferably at least 95% homologous to a polypeptide having the sequence YMLRYWSVGMDRVGLLIRENI (SEQ ID NO: 503) corresponding to amino acids 937-957 of H13410_P9 (SEQ ID NO: 141), wherein said first amino acid sequence, second amino acid sequence and third amino acid sequence are contiguous and in a sequential order.

### 4. Comparison Report Between H13410_P9 (SEQ ID NO: 141) and Q5T5C9_HUMAN (SEQ ID NO: 138):

A. An isolated chimeric polypeptide encoding for H13410_P9 (SEQ ID NO: 141), comprising a first amino acid sequence being at least about 70%, optionally at least 80%, preferably at least 85%, more preferably at least 90% and most preferably at least 95%, homologous to a polypeptide having the sequence MAEPGHSHHLSARVRGRTERRIPRLWRLLLWAGTAFQVTQGTGPELIIACKESEYHYEYTACDSTGSRWR VAVPHTPGLCTSLPDPVKGTECSFSCNAGEFLDMKDQSCKPCAEGRYSLGTGIRFDEWDELPHGFASLSAN MELDDSAAESTGNCTSSKWVPRGDYIASNTDECTATLMYAVNLKQSGTVNFEYYYPDSSIIFEFFVQNDQ CQPNADDSRWMKTTEKGWEFHSVELNRGNNVLYWRTTAFSVWTKVPKPVLVRNIAITGVAYTSECFPCK PGTYADKQGSSFCKLCPANSYSNKGETSCHQCDPDKYSEKGSSSCNVRPACTDKDYFYTHTACDANGET QL (SEQ ID NO: 501) corresponding to amino acids 1-350 of H13410_P9 (SEQ ID NO: 141), a second amino acid sequence being at least about 90% homologous to MYKWAKPKICSEDLEGAVKLPASGVKTHCPPCNPGFFKTNNSTCQPCPYGSYSNGSDCTRCPAGTEPAVG FEYKWWNTLPTNMETTVLSGINFEYKGMTGWEVAGDHIYTAAGASDNDFMILTLVVPGFRPPQSVMADT ENKEVARITFVFETLCSVNCELYFMVGVNSRINTPVETWKGSKGKQSYTYIIEENTTTSFTWAFQRTTFHE ASRKYTNDVAKIYSINVTNVMNGVASYCRPCALEASDVGSSCTSCPAGYYIDRDSGTCHSCP corresponding to amino acids 1-272 of Q5T5C9_HUMAN (SEQ ID NO: 138), which also corresponds to amino acids 351-622 of H13410_P9 (SEQ ID NO: 141), a bridging amino acid P corresponding to amino acid 623 of H13410_P9 (SEQ ID NO: 141), a third amino acid sequence being at least about 90% homologous to NTILKAHQPYGVQACVPCGPGTKNNKIHSLCYNDCTFSRNTPTRTFNYNFSALANTVTLAGGPSFTSKGLK YFHHFTLSLCGNQGRKMSVCTDNVTDLRIPEGESGFSKSITAYVCQAVIIPPEVTGYKAGVSSQPVSLADRL IGVTTDMTLDGITSPAELFHLESLGIPDVIFFYRSNDVTQSCSSGRSTTIRVRCSPQKTVPGSLLLPGTCSDGT CDGCNFHFLWESAAACPLCSVADYHAIVSSCVAGIQKTTYVWREPKLCSGGISLPEQRVTICKTIDFWLKV GISAGTCTAILLTVLTCYFWKKNQK corresponding to amino acids 274-586 of Q5T5C9_HUMAN (SEQ ID NO: 138), which also corresponds to amino acids 624-936 of H13410_P9 (SEQ ID NO: 141), and a fourth amino acid sequence being at least about 70%, optionally at least 80%, preferably at least 85%, more preferably at least 90% and most preferably at least 95% homologous to a polypeptide having the sequence YMLRYWSVGMDRVGLLIRENI (SEQ ID NO: 503) corresponding to amino acids 937-957 of H13410_P9 (SEQ ID NO: 141), wherein said first amino acid sequence, second amino acid sequence, bridging amino acid, third amino acid sequence and fourth amino acid sequence are contiguous and in a sequential order.

### 5. Comparison Report Between H13410_P9 (SEQ ID NO: 141) and Q9P2M2_HUMAN (SEQ ID NO: 135):

A. An isolated chimeric polypeptide encoding for H13410_P9 (SEQ ID NO: 141), comprising a first amino acid sequence being at least about 70%, optionally at least 80%, preferably at least 85%, more preferably at least 90% and most preferably at least 95%, homologous to a polypeptide having the sequence MAEPGHSHHLSARVRGRTERRIPRLWRLLLWAGTAFQVTQGTGPELHACKESEYHYEYTACDSTGSRWR VAVPHTPGLCTSLPDPVKGTECSFSCNAGEFLDMKDQSCKPCAEGRYSLGTGIRFDEWDELPHGFASLSAN MELDDSAAESTGNCTSSKWVPRGDYIASNTDECTATLMYAVNLKQSGTVNFEYYYPDSSIIFEFFVQNDQ CQPNADDSRWMKTTEKGWEFHSVELNRGNNVLYWRTTAFSVWTKVPKPVLVRNIAITGVAYTSECFPCK PGTYADKQGSSFCKLCPANSYSNKGETSCHQCDPDKYSEKGSSSCNVRPACTDKDYFYTHTACDANGET QLMYKWAKPKICSEDLEGAVKLPASGVKTHCPPCNPGFFKTNNSTCQPCPYGSYSNGSDCTRCPAGTEPA VGFEYK (SEQ ID NO: 502) corresponding to amino acids 1-424 of H13410_P9 (SEQ ID NO: 141), a second amino acid sequence being at least about 90% homologous to WWNTLPTNMETTVLSGINFEYKGMTGWEVAGDHIYTAAGASDNDFMILTLVVPGFRPPQSVMADTENKE VARITFVFETLCSVNCELYFMVGVNSRfNTPVETWKGSKGKQSYTYIIEENTTTSFTWAFQRTTFHEASRK YTNDVAKIYSINVTNVMNGVASYCRPCALEASDVGSSCTSCPAGYYIDRDSGTCHSCPPNTILKAHQPYGV QACVPCGPGTKNNKIHSLCYNDCTFSRNTPTRTFNYNFSALANTVTLAGGPSFTSKGLKYFHIIFTLSLCGN QGRKMSVCTDNVTDLRIPEGESGPSKSITAYVCQAVIIPPEVTGYKAGVSSQPVSLADRLIGVTTDMTLDGI TSPAELFHLESLGIPDVIFFYRSNDVTQSCSSGRSTTIRVRCSPQKTVPGSLLLPGTCSDGTCDGCNFHFLWE SAAACPLCSVADYHAIVSSCVAGIQKTTYVWREPKLCSGGISLPEQRVTICKTIDFWLKVGISAGTCTAILL TVLTCYFWKKNQK corresponding to amino acids 1-512 of Q9P2M2_HUMAN (SEQ ID NO: 135), which also corresponds to amino acids 425-936 of H13410_P9 (SEQ ID NO: 141), and a third amino acid sequence being at least about 70%, optionally at least 80%, preferably at least 85%, more preferably at least 90% and most preferably at least 95% homologous to a polypeptide having the sequence YMLRYWSVGMDRVGLLIRENI (SEQ ID NO: 503) corresponding to amino acids 937-957 of H13410_P9 (SEQ ID NO: 141), wherein said first amino acid sequence, second amino acid sequence and third amino acid sequence are contiguous and in a sequential order.

B. An isolated polypeptide encoding for a head of H13410_P9 (SEQ ID NO: 141), comprising a polypeptide being at least 70%, optionally at least about 80%, preferably at least about 85%, more preferably at least about 90% and most preferably at least about 95% homologous to the sequence

(SEQ ID NO: 502)

MAEPGHSHHLSARVRGRTERRIPRILWRLLLWAGTAFQVTQGTGPELHAC
KESEYHYEYTACDSTGSRWRVAVPHTPGLCTSLPDPVKGTECSFSCNAGE
FLDMKDQSCKPCAEGRYSLGTGIRFDEWDELPHGFASLSANMELDDSAAE
STGNCTSSKWVPRGDYIASNTDECTATLMYAVNLKQSGTVNFEYYYPDSS
IIFEFFVQNDQCQPNADDSRWMKTTEKGWEFHSVELNRGNNVLYWRTTAF
SVWTKVPKPVLVRNIAITGVAYTSECFPCKPGTYADKQGSSFCKLCPANS
YSNKGETSCHQCDPDKYSEKGSSSCNVRPACTDKDYFYTHTACDANGETQ
LMYKWAKPKICSEDLEGAVKLPASGVKTHCPPCNPGFFKTNNSTCQPCPY
GSYSNGSDCTRCPAGTEPAVGFEYK
of

(SEQ ID NO: 141)

H13410_P9.

The localization of the variant protein was determined according to results from a number of different software programs and analyses, including analyses from SignalP and other specialized programs. The variant protein is believed to be membranal.

Variant protein H13410_P9 (SEQ ID NO: 141) also has the following non-silent SNPs (Single Nucleotide Polymorphisms) as listed in Table 67, (given according to their position(s) on the amino acid sequence, with the alternative amino acid(s) listed).

TABLE 67
Amino acid mutations

| SNP position(s) on amino acid | |
|---|---|
| sequence | Alternative amino acid(s) |
| 16 | G -> * |
| 16 | G -> R |
| 86 | V -> I |
| 623 | P -> T |

Variant protein H13410_P9 (SEQ ID NO: 141) is encoded by the following transcript(s): H13410_T18 (SEQ ID NO: 89), for which coding portion starts at position 138 and ends at position 3008. The transcript also has the following SNPs as listed in Table 68 (given according to their position on the nucleotide sequence, with the alternative nucleic acid listed; H13410_P9 (SEQ ID NO: 141)).

TABLE 68
Nucleic acid SNPs

| | SNP position(s) on nucleotide |
|---|---|
| Polymorphism | sequence |
| G -> A | 183, 393 |
| G -> T | 183 |
| T -> C | 278, 2105, 2214 |
| C -> T | 1886, 3660, 2174 |
| C -> A | 2004 |
| A -> G | 2132, 2648 |
| C -> G | 3514 |
| C -> | 3514 |

Variant protein H13410_P20 (SEQ ID NO: 142) according to the present invention has an amino acid sequence as given in the “Sequence Listing” of the application; it is encoded by transcript(s) H13410 T35 (SEQ ID NO: 92).

The localization of the variant protein was determined according to results from a number of different software programs and analyses, including analyses from SignalP and other specialized programs. The variant protein is believed to be secreted.

Variant protein H13410_P20 (SEQ ID NO: 142) also has the following non-silent SNPs (Single Nucleotide Polymorphisms) as listed in Table 69, (given according to their position(s) on the amino acid sequence, with the alternative amino acid(s) listed).

TABLE 69
Amino acid mutations

| SNP position(s) on amino acid | |
|---|---|
| sequence | Alternative amino acid(s) |
| 16 | G -> * |
| 16 | G -> R |
| 86 | V -> I |

Variant protein H13410_P20 (SEQ ID NO: 142) is encoded by the following transcript(s): H13410_T35 (SEQ ID NO: 92), for which the coding portion starts at position 138 and ends at position 704. The transcript also has the following SNPs as listed in Table 70 (given according to their position on the nucleotide sequence, with the alternative nucleic acid listed).

TABLE 70
Nucleic acid SNPs

| | SNP position(s) on nucleotide |
|---|---|
| Polymorphism | sequence |
| G -> A | 183, 393 |
| G -> T | 183 |
| T -> C | 278 |

Variant protein H13410_P46 (SEQ ID NO: 143) according to the present invention has an amino acid sequence as given in the “Sequence Listing” of the application; it is encoded by transcript(s) H13410_T8 (SEQ ID NO: 87).

The localization of the variant protein was determined according to results from a number of different software programs and analyses, including analyses from SignalP and other specialized programs. The variant protein is believed to be secreted.

Variant protein H13410_P46 (SEQ ID NO: 143) also has the following non-silent SNPs (Single Nucleotide Polymorphisms) as listed in Table 71, (given according to their position(s) on the amino acid sequence, with the alternative amino acid(s) listed).

TABLE 71
Amino acid mutations

| SNP position(s) on amino acid | |
|---|---|
| sequence | Alternative amino acid(s) |
| 16 | G -> * |
| 16 | G -> R |
| 86 | V -> I |

Variant protein H13410_P46 (SEQ ID NO: 143) is encoded by the following transcript(s): H13410_T8 (SEQ ID NO: 87), for which the coding portion starts at position 138 and ends at position 1649. The transcript also has the following SNPs as listed in Table 72 (given according to their position on the nucleotide sequence, with the alternative nucleic acid listed).

TABLE 72
Nucleic acid SNPs

| | SNP position(s) on nucleotide |
|---|---|
| Polymorphism | sequence |
| G -> A | 183, 393 |
| G -> T | 183 |
| T -> C | 278, 2310, 2201 |
| C -> T | 1982, 2270, 3259 |
| C -> A | 2100 |
| A -> G | 2228, 2744 |
| C -> G | 3113 |
| C -> | 3113 |

Variant protein H13410_P47 (SEQ ID NO: 144) according to the present invention has an amino acid sequence as given in the “Sequence Listing” of the application; it is encoded by transcript(s) H13410_T21 (SEQ ID NO: 90).

The localization of the variant protein was determined according to results from a number of different software programs and analyses, including analyses from SignalP and other specialized programs. The variant protein is believed to be secreted.

Variant protein H13410_P47 (SEQ ID NO: 144) also has the following non-silent SNPs (Single Nucleotide Polymorphisms) as listed in Table 73, (given according to their position(s) on the amino acid sequence, with the alternative amino acid(s) listed).

TABLE 73
Amino acid mutations

| SNP position(s) on amino acid | |
|---|---|
| sequence | Alternative amino acid(s) |
| 16 | G -> * |
| 16 | G -> R |
| 86 | V -> I |

Variant protein H13410_P47 (SEQ ID NO: 144) is encoded by the following transcript(s): H13410_T21 (SEQ ID NO: 90), for which the coding portion starts at position 138 and ends at position 1127. The transcript also has the following SNPs as listed in Table 74 (given according to their position on the nucleotide sequence, with the alternative nucleic acid listed).

TABLE 74
Nucleic acid SNPs

| | SNP position(s) on nucleotide |
|---|---|
| Polymorphism | sequence |
| G -> A | 183, 393 |
| G -> T | 183 |
| T -> C | 278, 2112, 2221 |
| C -> T | 1893, 3170, 2181 |
| C -> A | 2011 |
| A -> G | 2139, 2655 |
| C -> G | 3024 |
| C -> | 3024 |

As noted above, cluster H13410 features 40 segment(s), which were listed in Table 59 above and for which the sequence(s) are given in the “Sequence Listing” of the application. These segment(s) are portions of nucleic acid sequence(s) which are described herein separately because they are of particular interest. A description of some of these segments according to the present invention is now provided.

Segment cluster H134101N20 (SEQ ID NO: 97) according to the present invention is supported by 29 libraries. The number of libraries was determined as previously described. This segment can be found in the following transcript(s): H13410_T12 (SEQ ID NO: 88), H13410_T18 (SEQ ID NO: 89), H13410_T21 (SEQ ID NO: 90), H13410_T27 (SEQ ID NO: 91) and H13410_T8 (SEQ ID NO: 87). Table 75 below describes the starting and ending position of this segment on each transcript.

TABLE 75
Segment location on transcripts

| | Segment | Segment |
|---|---|---|
| Transcript name | starting position | ending position |
| H13410_T12 (SEQ ID NO: 88) | 940 | 1089 |
| H13410_T18 (SEQ ID NO: 89) | 940 | 1089 |
| H13410_T21 (SEQ ID NO: 90) | 940 | 1089 |
| H13410_T27 (SEQ ID NO: 91) | 940 | 1089 |
| H13410_T8 (SEQ ID NO: 87) | 940 | 1089 |

Segment cluster H13410_N27 (SEQ ID NO: 98) according to the present invention is supported by 40 libraries. The number of libraries was determined as previously described. This segment can be found in the following transcript(s): H13410_T12 (SEQ ID NO: 88), H13410_T18 (SEQ ID NO: 89), H13410_T21 (SEQ ID NO: 90), H13410_T27 (SEQ ID NO: 91) and H13410_T8 (SEQ ID NO: 87). Table 76 below describes the starting and ending position of this segment on each transcript.

TABLE 76
Segment location on transcripts

| | Segment | Segment |
|---|---|---|
| Transcript name | starting position | ending position |
| H13410_T12 (SEQ ID NO: 88) | 1179 | 1356 |
| H13410_T18 (SEQ ID NO: 89) | 1179 | 1356 |
| H13410_T21 (SEQ ID NO: 90) | 1090 | 1267 |
| H13410_T27 (SEQ ID NO: 91) | 1179 | 1356 |
| H13410_T8 (SEQ ID NO: 87) | 1179 | 1356 |

Segment cluster H13410_N36 (SEQ ID NO: 100) according to the present invention is supported by 1 libraries. The number of libraries was determined as previously described. This segment can be found in the following transcript(s): H13410_T12 (SEQ ID NO: 88). Table 77 below describes the starting and ending position of this segment on each transcript.

TABLE 77
Segment location on transcripts

| | Segment | Segment |
|---|---|---|
| Transcript name | starting position | ending position |
| H13410_T12 (SEQ ID NO: 88) | 1683 | 1936 |

Segment cluster H13410_N58 (SEQ ID NO: 106) according to the present invention is supported by 2 libraries. The number of libraries was determined as previously described. This segment can be found in the following transcript(s): H13410_T18 (SEQ ID NO: 89) and H13410_T27 (SEQ ID NO: 91). Table 78 below describes the starting and ending position of this segment on each transcript.

TABLE 78
Segment location on transcripts

| | Segment | Segment |
|---|---|---|
| Transcript name | starting position | ending position |
| H13410_T18 (SEQ ID NO: 89) | 2957 | 3441 |
| H13410_T27 (SEQ ID NO: 91) | 3035 | 3519 |

According to an optional embodiment of the present invention, short segments related to the above cluster are also provided. These segments are up to about 120 bp in length, and so are included in a separate description.

Segment cluster H13410_N12 (SEQ ID NO: 112) according to the present invention is supported by 1 libraries. The number of libraries was determined as previously described. This segment can be found in the following transcript(s): H13410_T35 (SEQ ID NO: 92). Table 79 below describes the starting and ending position of this segment on each transcript.

TABLE 79
Segment location on transcripts

| | Segment | Segment |
|---|---|---|
| Transcript name | starting position | ending position |
| H13410_T35 (SEQ ID NO: 92) | 605 | 723 |

Segment cluster H13410_N33 (SEQ ID NO: 117) according to the present invention is supported by 3 libraries. The number of libraries was determined as previously described. This segment can be found in the following transcript(s): H13410_T21 (SEQ ID NO: 90) and H13410_T8 (SEQ ID NO: 87). Table 80 below describes the starting and ending position of this segment on each transcript.

TABLE 80
Segment location on transcripts

| | Segment | Segment |
|---|---|---|
| Transcript name | starting position | ending position |
| H13410_T21 (SEQ ID NO: 90) | 1488 | 1583 |
| H13410_T8 (SEQ ID NO: 87) | 1577 | 1672 |

Segment cluster H13410_N55 (SEQ ID NO: 125) according to the present invention is supported by 2 libraries. The number of libraries was determined as previously described. This segment can be found in the following transcript(s): H13410T27 (SEQ ID NO: 91). Table 81 below describes the starting and ending position of this segment on each transcript.

TABLE 81
Segment location on transcripts

| | Segment | Segment |
|---|---|---|
| Transcript name | starting position | ending position |
| H13410_T27 (SEQ ID NO: 91) | 2766 | 2843 |

Expression of Homo sapiens maba1 (SEQ ID NO: 133) transcripts detectable by or according to the following segments was measured by real time PCR: seg10WTF2R2—H13410_seg10WTF2R2 (SEQ ID NO: 234) amplicon and primers H13410_seg10WTF2 (SEQ ID NO: 232) and H13410_seg10WTR2 (SEQ ID NO: 233); seg12—H13410_seg12 (SEQ ID NO: 237) amplicon and primers H13410_seg12F (SEQ ID NO: 235) and H13410_seg12R (SEQ ID NO: 236); seg33—H13410_seg33 (SEQ ID NO: 240) amplicon and primers H13410_seg33F (SEQ ID NO: 238) and H13410_seg33R (SEQ ID NO: 239); seg58—H13410_seg58 (SEQ ID NO: 243) amplicon and primers H13410_seg58F (SEQ ID NO: 241) and H13410_seg58R (SEQ ID NO: 242); junc20-27F2R2-H13410_junc20-27F2R2 (SEQ ID NO: 385) amplicon and primers H13410_junc20-27F2 (SEQ ID NO: 383) and H13410_junc20-27R2 (SEQ ID NO: 384). The sequences of corresponding primers and amplicans are given below:

Forward Primer H13410_seg10WTF2 (SEQ ID NO: 232):
CTACTCCCTCGGCACAGGC
Reverse Primer H13410_seg10WTR2 (SEQ ID NO: 233):
CAGCAGCACTGTCATCCAGC
Amplicon H13410_seg10WTF2R2 (SEQ ID NO: 234):
CTACTCCCTCGGCACAGGCATTCGGTTTGATGAGTGGGATGAGCTGCCCC
ATGGCTTTGCCAGCCTCTCAGCCAACATGGAGCTGGATGACAGTGCTGCT
G
Forward Primer H13410_seg12F (SEQ ID NO: 235):
ACGAGGAACTGGAAGCTCAGC
Reverse Primer H13410_seg12R (SEQ ID NO: 236):
AATGTGGCATTATGGAGCAGTG
Amplicon H13410_seg12 (SEQ ID NO: 237):
ACGAGGAACTGGAAGCTCAGCAAGGTTATGACCTGCTCTTTGTTATCCGG
TTTACAGCCGGCGAGATTGAAATTCGAACCCAAGCTTCCACTGCTCCATA
ATGCCACATT
Forward Primer H13410_seg33F (SEQ ID NO: 238))
GTCCCTGCTTCCCATCCCT
Reverse Primer H13410_seg33R (SEQ ID NO: 239):
CTTGCAAACACAGGCTTCATTC
Amplicon H13410_seg33 (SEQ ID NO: 240):
GTCCCTGCTTCCCATCCCTGGGATGTTGCTCAGTTTGCCTTCCCTTAACA
CACATTACTCAGAGCTAGCCAGATGAATGAAGCCTGTGTTTGCAAG
Forward Primer H13410_seg58F (SEQ ID NO: 241):
AAAGATCACTGAACTGGGAACTAAGAT
Reverse Primer H13410_seg58R (SEQ ID NO: 242):
GGTCCTATTCCCATGCCATG
Amplicon H13410_seg58 (SEQ ID NO: 243):
AAAGATCACTGAACTGGGAACTAAGATTCCTTCTATTACCCCAGCCCTGA
CACCATTTTACCATGCTTCAGTCTCCTTACCCATGGCATGGGAATAGGAC
C
Forward Primer H13410_junc20-27F2
(SEQ ID NO: 383)):
TTCTGCAAACTTTGCCCAGC
Reverse Primer H13410_junc20-27R2
(SEQ ID NO: 384)):
CCATTTGTACATGAGTTGTGTCTGAG
Amplicon H13410_junc20-27F2R2 (SEQ ID NO: 385)):
TTCTGCAAACTTTGCCCAGCCAACTCTTATTCAAATAAAGGAGAAACTTC
TTGCCACCAGTGTGACCCTGACAAATACTCAGACACAACTCATGTACAAA
TGG

Expression of Homo sapiens maba1 (SEQ ID NO: 133) (KIAA1324) H13410 Transcripts which are Detectable by Amplicon as Depicted in Sequence Name H13410_seg10WTF2R2 (SEQ ID NO: 234) in Normal and Cancerous Breast Tissues

Expression of Homo sapiens maba1 (KIAA1324) transcripts detectable by or according to seg10WTF2R2-H13410_seg10WTF2R2 (SEQ ID NO: 234) amplicon and primers H13410_seg10WTF2 (SEQ ID NO: 232) and H13410_seg10WTR2 (SEQ ID NO: 233) was measured by real time PCR on 2 breast RT panels: one panel (Table 1_{—}3) was checked for the expression of four housekeeping genes—G6PD (GenBank Accession No. NM_{—}000402 (SEQ ID NO:339); G6PD amplicon (SEQ ID NO: 311)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)). Then, for each RT sample, the expression of the above amplicon was normalized to the geometric mean of the quantities of the housekeeping genes. The normalized quantity of each RT sample was then divided by the median of the quantities of the normal post-mortem (PM) samples (sample numbers 57, 59, 60, 63, 66, 64, 56, 65, 67 and 58, Table 1_{—}3 above), to obtain a value of fold up-regulation for each sample relative to the median of the normal PM samples. The second RT panel (Table 1_{—}7) was checked for the expression of the following housekeeping genes—G6PD (GenBank Accession No. NM_{—}000402 (SEQ ID NO:339); G6PD amplicon (SEQ ID NO: 311)), RPL19 (GenBank Accession No. NM_{—}000981 (SEQ ID NO:342); RPL19 amplicon (SEQ ID NO: 320)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)). Then, for each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 43, 45, 46, 47, 48, 49, 50, 51, 52, 54, 56, 58, 59, 60, 61, 62, 63, 64, 65, 66, 67, 68 and 69, Table 1_{—}7 above), to obtain a value of fold up-regulation for each sample relative to the median of the normal samples.

In one experiment with each panel no significant differential expression in the cancerous samples relative to the normal samples was observed.

Primer pairs are also optionally and preferably encompassed within the present invention; for example, for the above experiment, the following primer pair was used as a non-limiting illustrative example only of a suitable primer pair: H13410_seg10WTF2 (SEQ ID NO: 232) forward primer; and H13410_seg10WTR2 (SEQ ID NO: 233) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg10WTF2R2 (SEQ ID NO: 234).

Expression of Homo sapiens maba1 (KIAA1324) H13410 Transcripts which are Detectable by Amplicon as Depicted in Sequence Name H13410_seg10WTF2R2 (SEQ ID NO: 234) in Normal and Cancerous Lung Tissues

Expression of Homo sapiens maba1 (KIAA1324) transcripts detectable by or according to seg10WTF2R2-H13410_seg10WTF2R2 (SEQ ID NO: 234) amplicon and primers H13410_seg10WTF2 (SEQ ID NO: 232) and H13410_seg10WTR2 (SEQ ID NO: 233) was measured by real time PCR. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 51, 52, 53, 54, 55, 56, 57, 58, 59, 60, 61, 62, 63, 64, 69 and 70, Table 1_{—}6 above), to obtain a value of fold up-regulation for each sample relative to the median of the normal samples.

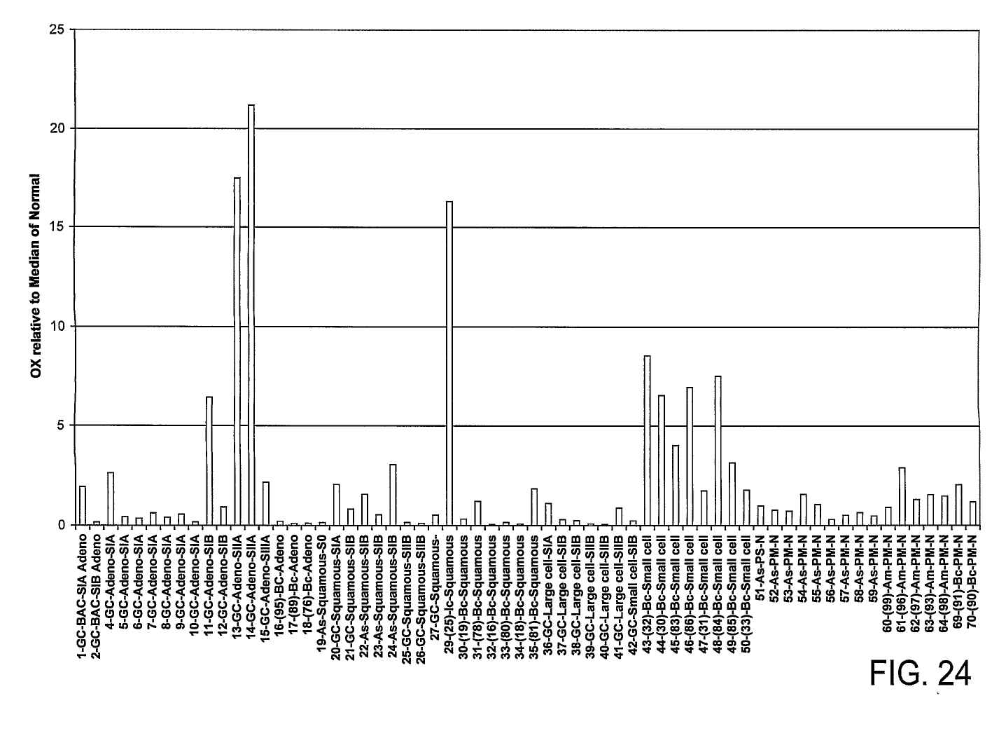

FIG. 24 is a histogram showing over expression of the above-indicated Homo sapiens maba1 (KIAA1324) transcripts in cancerous Lung samples relative to the normal samples.

As is evident from FIG. 24, the expression of Homo sapiens maba1 (KIAA1324) transcripts detectable by the above amplicon in small cell carcinoma samples was significantly higher than in the non-cancerous samples (sample numbers 51, 52, 53, 54, 55, 56, 57, 58, 59, 60, 61, 62, 63, 64, 69 and 70, Table 1_{—}6 above) and was higher in a few non-small cell carcinoma samples than in the non-cancerous samples. Notably an over-expression of at least 5 fold was found in 4 out of 9 small cell carcinoma samples.

Statistical analysis was applied to verify the significance of these results, as described below.

The P value for the difference in the expression levels of Homo sapiens maba1 (KIAA1324) transcripts detectable by the above amplicon in Lung small cell carcinoma samples versus the normal tissue samples was determined by T test as 9.54e-003.

Threshold of 5 fold over expression was found to differentiate between small cell carcinoma and normal samples with P value of 9.96e-003 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H13410_seg10WTF2 (SEQ ID NO: 232) forward primer; and H13410_seg10WTR2 (SEQ ID NO: 233) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg10WTF2R2 (SEQ ID NO: 234).

Expression of Homo sapiens maba1 H13410 Transcripts which are Detectable by Amplicon as Depicted in Sequence Name H13410_seg10WTF2R2 (SEQ ID NO: 234) in Normal and Cancerous Ovary Tissues

Expression of Homo sapiens maba1 transcripts detectable by or according to seg10WTF2R2-H13410_seg10WTF2R2 (SEQ ID NO: 234) amplicon and primers H13410_seg10WTF2 (SEQ ID NO: 232) and H13410_seg10WTR2 (SEQ ID NO: 233) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon —HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and GAPDH (GenBank Accession No. BC026907 (SEQ ID NO:338); GAPDH amplicon (SEQ ID NO: 308)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 45, 46, 71 and 48, Table 1_{—}1 above), to obtain a value of fold up-regulation for each sample relative to the median of the normal PM samples.

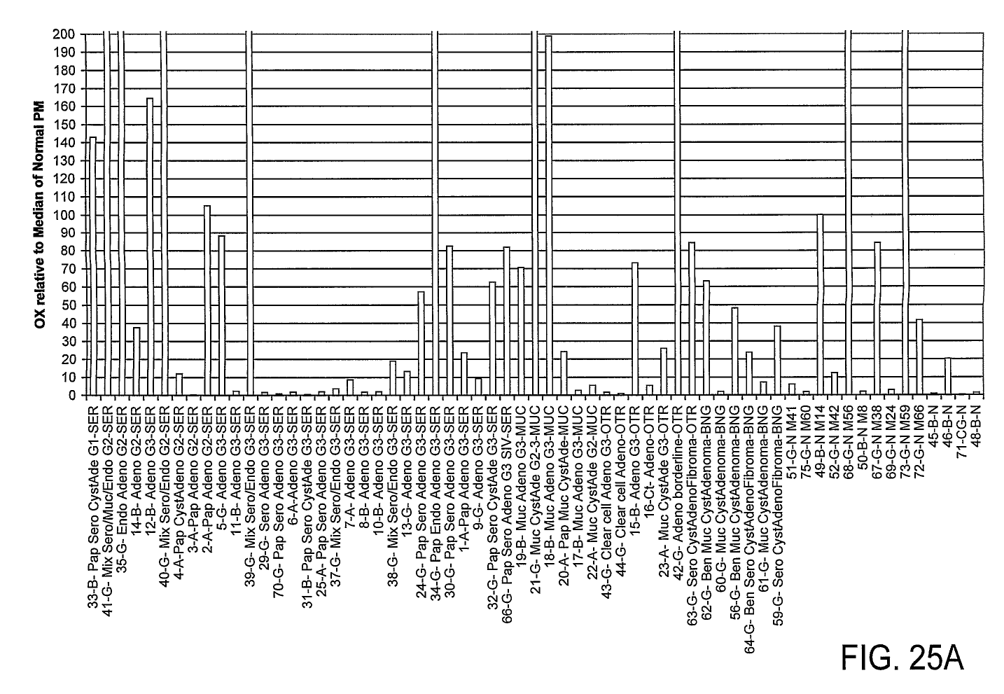

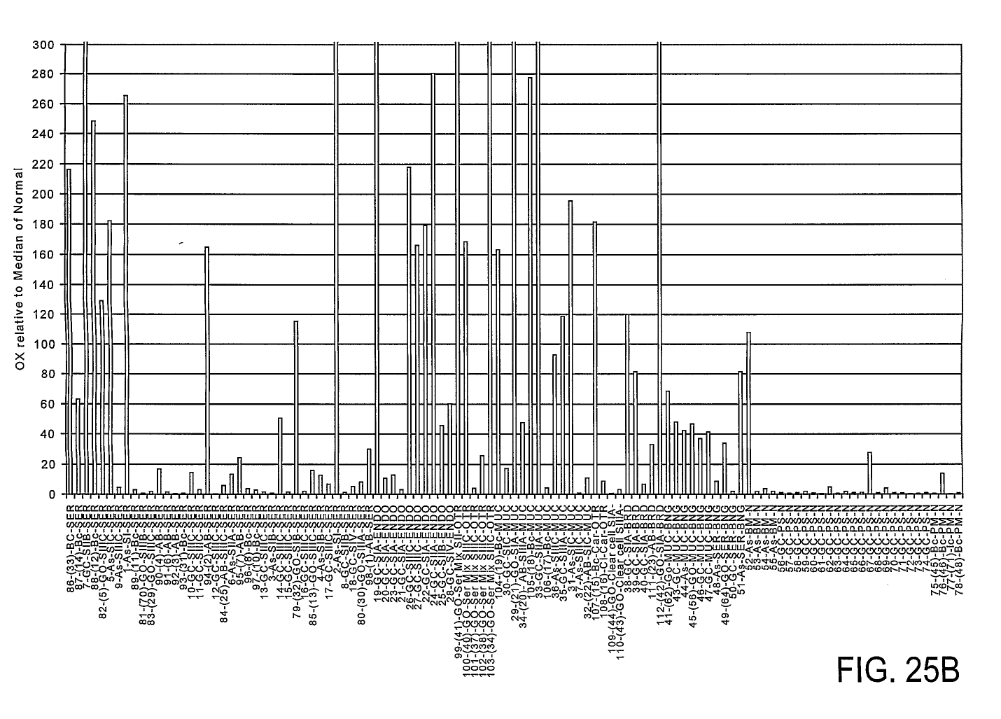

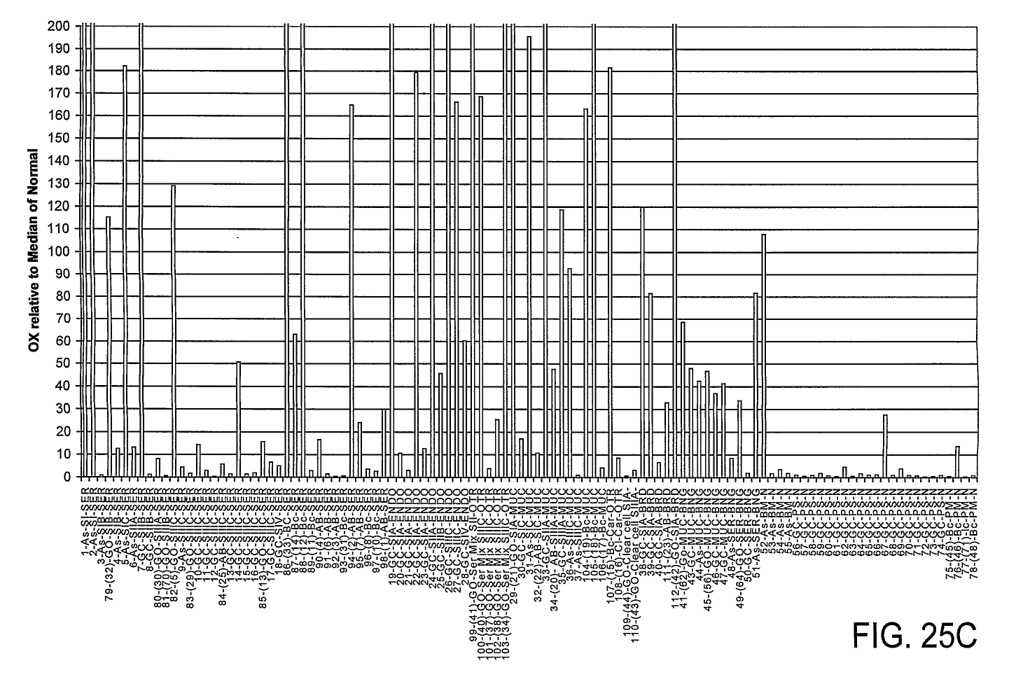

FIG. 25A is a histogram showing over expression of the Homo sapiens maba1 transcripts detectable by or according to H13410_seg10WTF2R2 (SEQ ID NO: 234) in cancerous Ovary samples relative to the normal samples.

As is evident from FIG. 25A, the expression of Homo sapiens maba1 transcripts detectable by the above amplicon in adenocarcinoma samples was higher than in the non-cancerous samples (sample numbers 45, 46, 71 and 48, Table 1_{—}1 above). Notably an over-expression of at least 5 fold was found in 20 out of 30 serous carcinoma samples and in 5 out of 6 mucinous carcinoma samples. Overall, there is over-expression in 30 samples out of 43 adenocarcinoma samples.

Statistical analysis was applied to verify the significance of these results, as described below.

The P value for the difference in the expression levels of Homo sapiens maba1 transcripts detectable by the above amplicon in Ovary adenocarcinoma samples versus the normal tissue samples was determined by T test as 1.25e-003.

The P value for the difference in the expression levels of Homo sapiens maba1 transcripts detectable by the above amplicon in Ovary serous carcinoma samples versus the normal tissue samples was determined by T test as 1.04e-002. The above values demonstrate statistical significance of the results.

Primer pairs are also optionally and preferably encompassed within the present invention; for example, for the above experiment, the following primer pair was used as a non-limiting illustrative example only of a suitable primer pair: H13410_seg10WTF2 (SEQ ID NO: 232) forward primer; and H13410_seg10WTR2 (SEQ ID NO: 233) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg10WTF2R2 (SEQ ID NO: 234).

Expression of Homo sapiens maba1 (KIAA1324) transcripts detectable by or according to seg10WTF2R2-H13410_seg10WTF2R2 (SEQ ID NO: 234) amplicon and primers H13410_seg10WTF2 (SEQ ID NO: 232) and H13410_seg10WTR2 (SEQ ID NO: 233) was further measured by real time PCR using enlarged panel as shown in Table 1_{—}5. In parallel the expression of three housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)) and G6PD (GenBank Accession No. NM_{—}000402 (SEQ ID NO:339); G6PD amplicon (SEQ ID NO: 311)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above), to obtain a value of fold up-regulation for each sample relative to the median of the normal samples.

FIGS. 25B and 25C are histograms showing over expression of the above-indicated Homo sapiens maba1 (KIAA1324) transcripts in cancerous Ovary samples relative to the normal samples. In FIG. 25B the samples are arranged according to the patient's age, and in FIG. 25C samples are arranged according to the patient's stage.

As is evident from FIGS. 25B and 25C, the expression of Homo sapiens maba1 (KIAA1324) transcripts detectable by the above amplicon in serous carcinoma, mucinous carcinoma and adenocarcinoma samples was significantly higher than in the non-cancerous samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above). Notably an over-expression of at least 5 fold was found in 47 out of 69 adenocarcinoma samples in 26 out of 43 serous carcinoma samples, in 10 out of 12 mucinous carcinoma samples and in 9 out of 10 endometroid samples. From FIG. 25B it can be observed that in serous samples over expression was mainly in patients with age below 50, in mucinus samples over expression was higher in patients with age below 50 and in endometroid over expression was mainly in patients with age above 50. From FIG. 25C it can be seen that in serous samples over expression was mainly in patients with stage I and II.

Statistical analysis was applied to verify the significance of these results, as described below.

The P value for the difference in the expression levels of Homo sapiens maba1 (KIAA1324) transcripts detectable by the above amplicon in Ovary adenocarcinoma samples versus the normal tissue samples was determined by T test as 1.70e-005. The P value for the difference in the expression levels of Homo sapiens maba1 (KIAA1324) transcripts detectable by the above amplicon in Ovary serous carcinoma samples versus the normal tissue samples was determined by T test as 2.18e-003. The P value for the difference in the expression levels of Homo sapiens maba1 (KIAA1324) transcripts detectable by the above amplicon in Ovary mucinous carcinoma samples versus the normal tissue samples was determined by T test as 3.94e-002. The P value for the difference in the expression levels of Homo sapiens maba1 (KIAA1324) transcripts detectable by the above amplicon in Ovary endometroid samples versus the normal tissue samples was determined by T test as 2.76e-002.

Threshold of 5 fold over expression was found to differentiate between adenocarcinoma and normal samples with P value of 3.06e-007 as checked by exact Fisher test. Threshold of 5 fold over expression was found to differentiate between serous carcinoma and normal samples with P value of 3.27e-005 as checked by exact Fisher test. Threshold of 5 fold over expression was found to differentiate between mucinous carcinoma and normal samples with P value of 2.40e-005 as checked by exact Fisher test. Threshold of 5 fold over expression was found to differentiate between endometroid and normal samples with P value of 1.58e-005 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H13410_seg10WTF2 (SEQ ID NO: 232) forward primer; and H13410_seg10WTR2 (SEQ ID NO: 233) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg10WTF2R2 (SEQ ID NO: 234).

Expression of Homo sapiens Maba1 H13410 Transcripts which are Detectable by Amplicon as Depicted in Sequence Name H13410_seg10WTF2R2 (SEQ ID NO: 234) in Different Normal Tissues

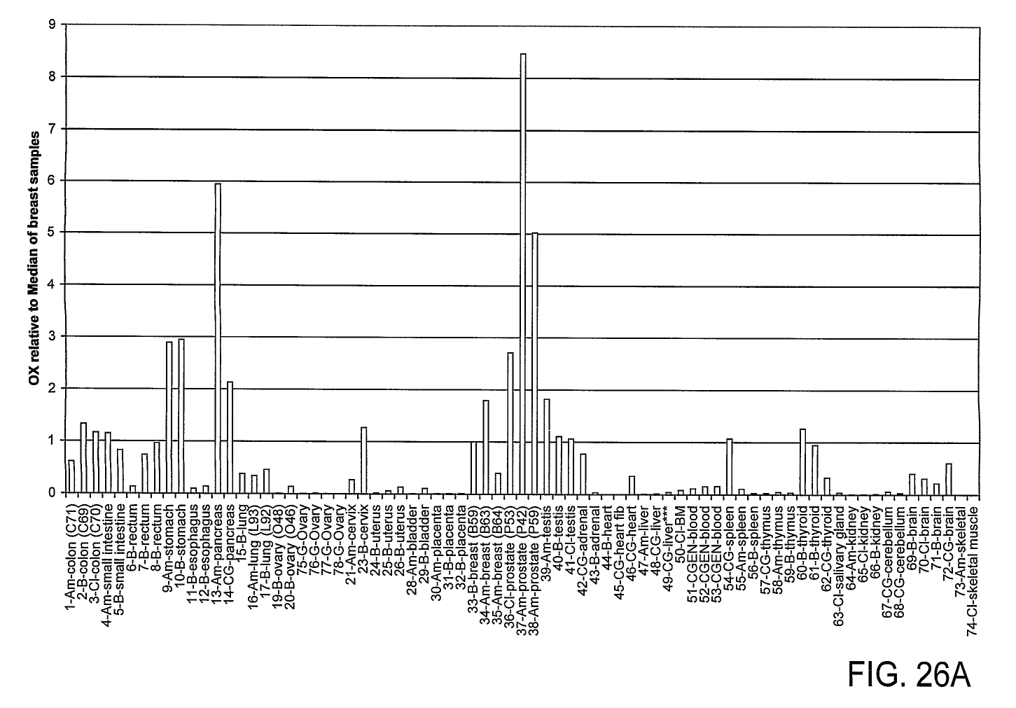

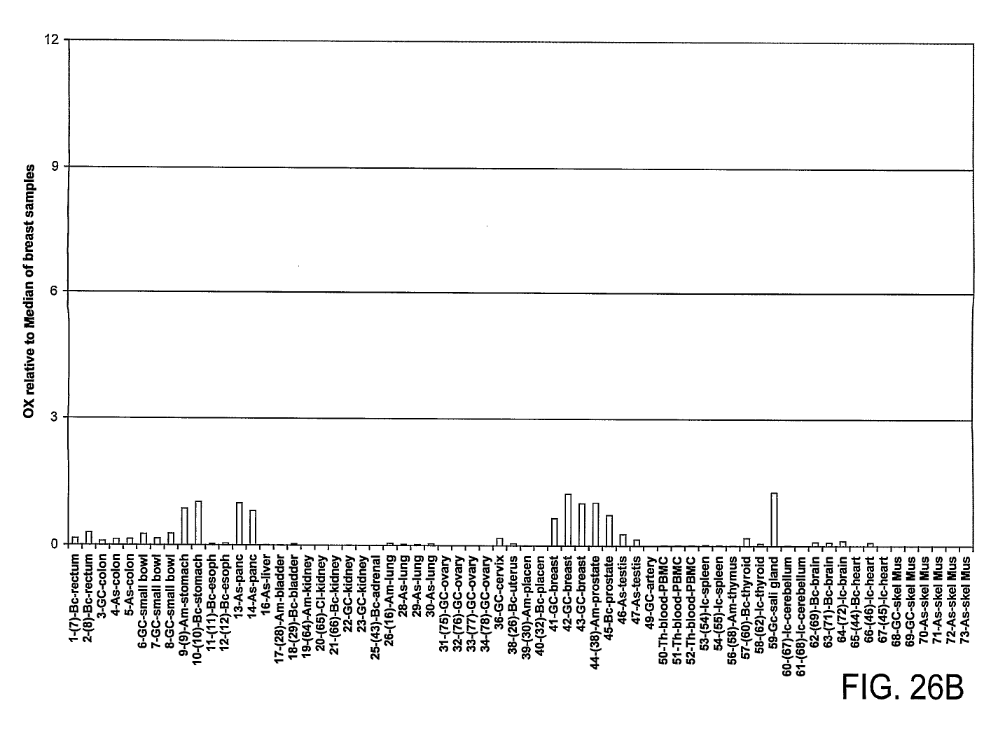

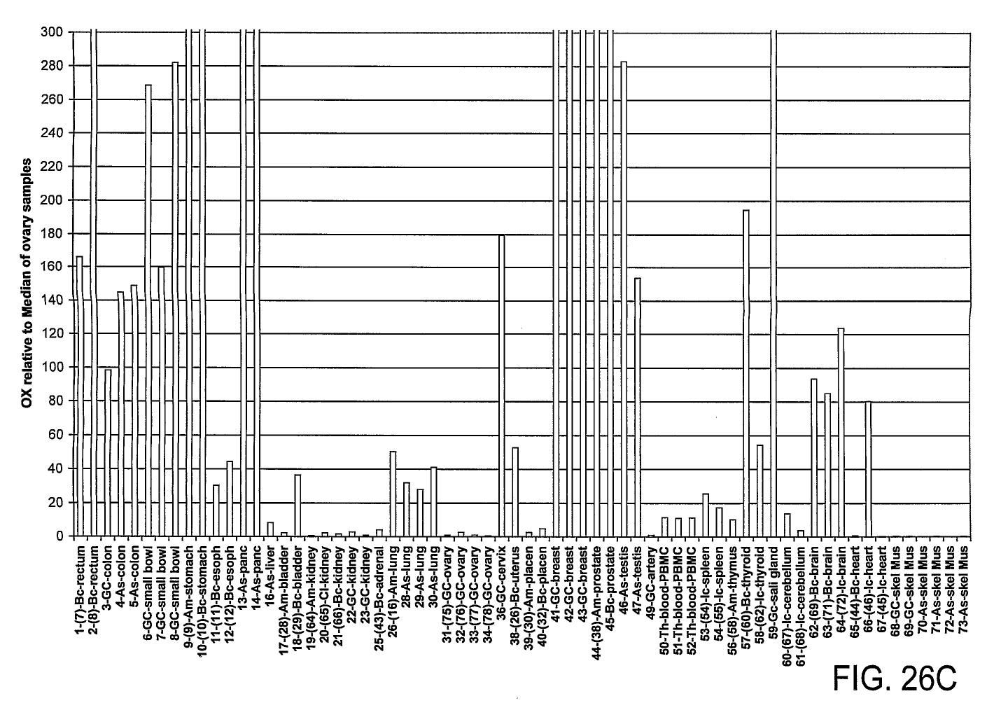

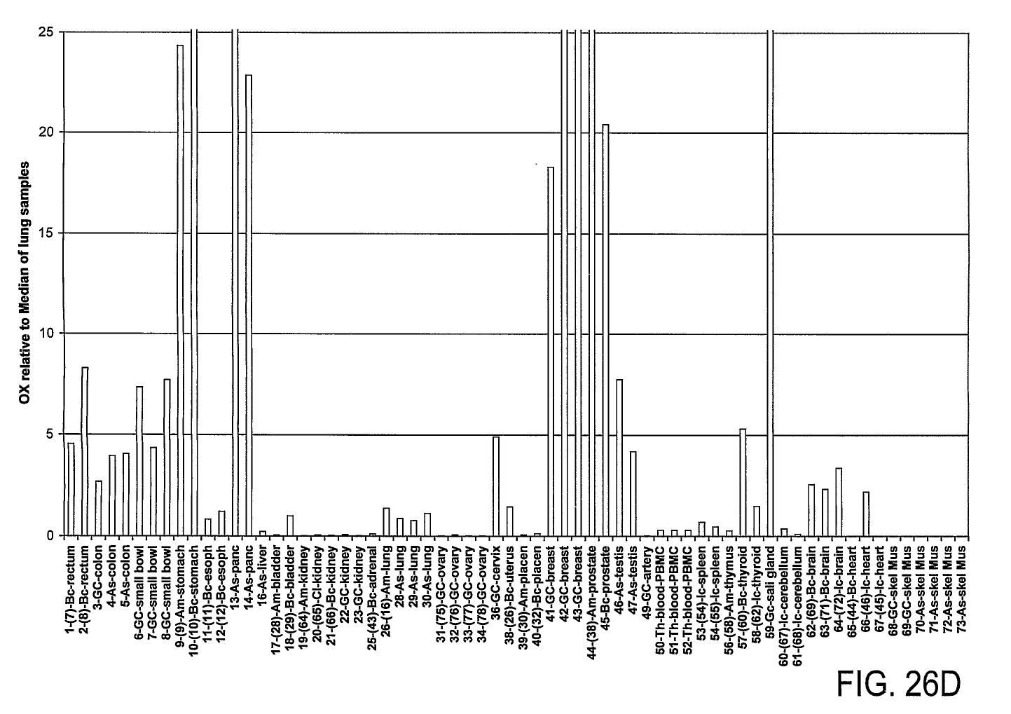

Expression of Homo sapiens maba1 transcripts detectable by or according to seg10WTF2R2-H13410_seg10WTF2R2 (SEQ ID NO: 234) amplicon and primers H13410_seg10WTF2 (SEQ ID NO: 232) and H13410_seg10WTR2 (SEQ ID NO: 233) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the geometric mean of the quantities of the housekeeping genes. The normalized quantity of each RT sample was then divided by the median of the quantities of the breast samples (sample numbers 33, 34 and 35, Table 1_{—}9 above), to obtain a value of relative expression of each sample relative to the median of the breast samples. FIG. 26A shows expression of Homo sapiens maba1 H13410 transcripts which are detectable by amplicon as depicted in sequence name H13410_seg10WTF2R2 (SEQ ID NO: 234) in different normal tissues.

Expression of Homo sapiens maba1 (KIAA1324) transcripts detectable by or according to seg10WTF2R2-H13410_seg10WTF2R2 (SEQ ID NO: 234) amplicon and primers H13410_seg10WTF2R2F (SEQ ID NO: 232) and H13410_seg10WTF2R2R (SEQ ID NO: 233) was further measured by real time PCR using updated panel presented in table 1_{—}10. In parallel the expression of three housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), and TATA box (GenBank Accession No. NM_{—}003194 (SEQ ID NO:343); TATA amplicon (SEQ ID NO: 323)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the breast samples (sample numbers 41, 42 and 43, Table 1_{—}10 above), to obtain a value of relative expression of each sample relative to the median of the breast samples—as presented in FIG. 26B, or to the median of the quantities of the ovary samples (sample numbers 31, 32, 33 and 34, Table 1_{—}10 above), to obtain a value of relative expression of each sample relative to the median of the ovary samples—as presented in FIG. 26C, or the median of the quantities of the lung samples (sample numbers 28, 29 and 30, Table 1_{—}10 above), to obtain a value of relative expression of each sample relative to the median of the lung samples—as presented in FIG. 26D.

FIG. 26 is a histogram showing expression of Homo sapiens maba1 (KIAA1324) H13410 transcripts which are detectable by amplicon as depicted in sequence name H13410_seg10WTF2R2 (SEQ ID NO: 234) in different normal tissues.

Expression of Homo sapiens maba1 H13410 Transcripts which are Detectable by Amplicon as Depicted in Sequence Name H13410_seg12 (SEQ ID NO: 237) in Normal and Cancerous Breast Tissues.

Expression of Homo sapiens maba1 transcripts detectable by or according to seg12-H13410_seg12 (SEQ ID NO: 237) amplicon and primers H13410_seg12F (SEQ ID NO: 235) and H13410_seg12R (SEQ ID NO: 236) was measured by real time PCR. In parallel the expression of four housekeeping genes—G6PD (GenBank Accession No. NM_{—}000402 (SEQ ID NO:339); G6PD amplicon (SEQ ID NO: 311)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336) (amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 57, 59, 60, 63, 66, 64, 56, 65, 67 and 58, Table 1_{—}3 above: “Tissue samples in breast cancer testing panel”), to obtain a value of fold up-regulation for each sample relative to the median of the normal PM samples.

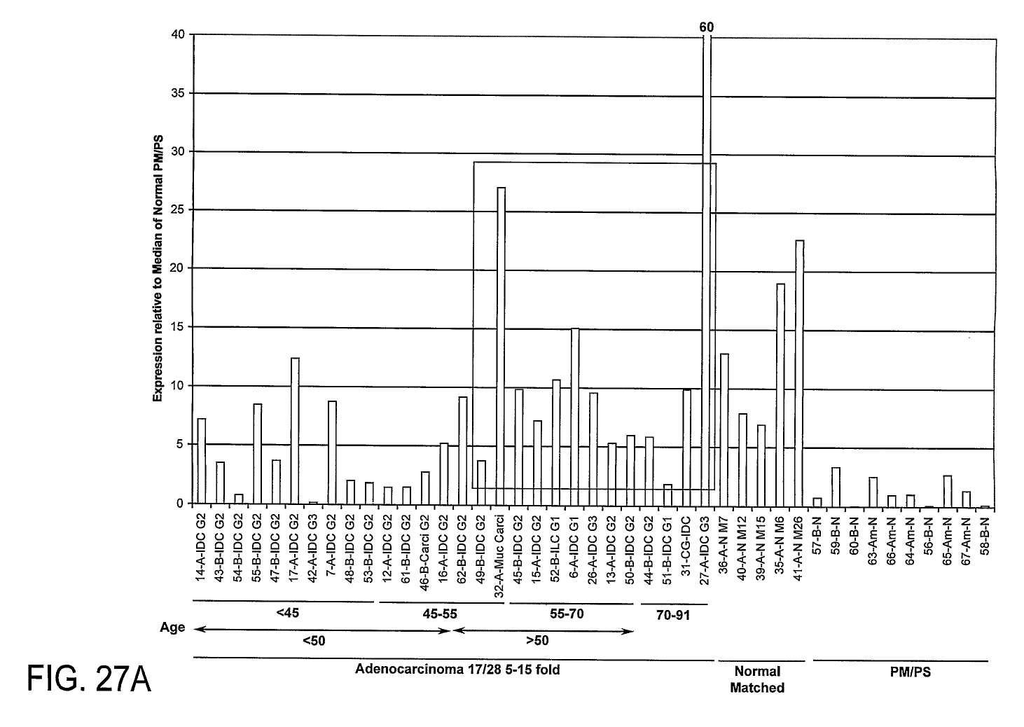

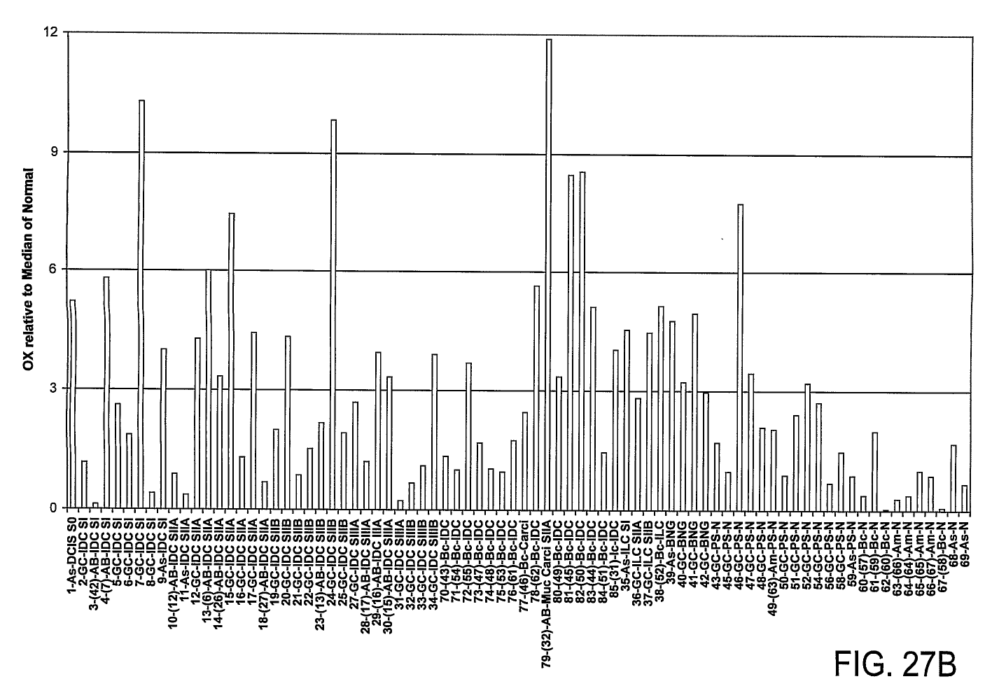

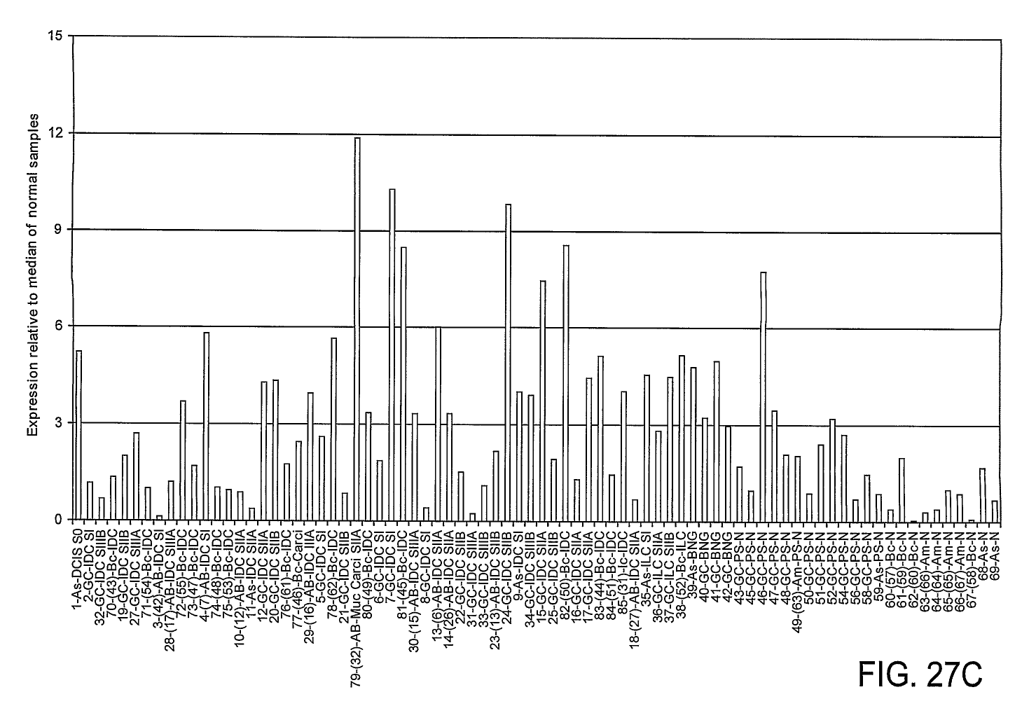

FIG. 27A is a histogram showing over expression of the Homo sapiens maba1 transcripts in cancerous Breast samples relative to the normal samples.

As is evident from FIG. 27A, the expression of Homo sapiens maba1 transcripts detectable by the above amplicon in cancer samples was significantly higher than in the non-cancerous samples (sample numbers 57, 59, 60, 63, 66, 64, 56, 65, 67 and 58, Table 1_{—}3 above: “Tissue samples in breast cancer testing panel”), especially in samples from patients with age above 50. Notably an over-expression of at least 5 fold was found in 17 out of 28 adenocarcinoma samples.

Statistical analysis was applied to verify the significance of these results, as described below.

The P value for the difference in the expression levels of Homo sapiens maba1 transcripts detectable by the above amplicon in Breast cancer samples versus the normal tissue samples was determined by T test as 2.24e-003.

Threshold of 5 fold over expression was found to differentiate between cancer and normal samples with P value of 7.46e-004 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H13410_seg12F (SEQ ID NO: 235) forward primer; and H13410_seg12R (SEQ ID NO: 236) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg12 (SEQ ID NO: 237).

Expression of Homo sapiens maba1 (KIAA1324) transcripts detectable by or according to seg12-H13410_seg12 (SEQ ID NO: 237) amplicon and primers H13410_seg12F (SEQ ID NO: 235) and H13410_seg12R (SEQ ID NO: 236) was further measured by real time PCR using enlarged panel as shown in Table 1_{—}7. In parallel the expression of four housekeeping genes—G6PD (GenBank Accession No. NM_{—}000402 (SEQ ID NO:339); G6PD amplicon (SEQ ID NO: 311)), RPL19 (GenBank Accession No. NM_{—}000981 (SEQ ID NO:342); RPL19 amplicon (SEQ ID NO: 320)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon —SDHA-amplicon (SEQ ID NO:299)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 43, 45, 46, 47, 48, 49, 50, 51, 52, 54, 56, 58, 59, 60, 61, 62, 63, 64, 65, 66, 67, 68 and 69, Table 1_{—}7 above), to obtain a value of fold up-regulation for each sample relative to the median of the normal samples.

FIGS. 27B and 27C are a histograms showing over expression of the above-indicated Homo sapiens maba1 (KIAA1324) transcripts in cancerous Breast samples relative to the normal samples. In FIG. 27B—the cancer samples are arranged according to the stage of the cancer from the patient, and in FIG. 27C the samples are arranged according to patient's age.

As is evident from FIGS. 27B and 27C, the expression of Homo sapiens maba1 (KIAA1324) transcripts detectable by the above amplicon in cancer samples was significantly higher than in the non-cancerous samples (sample numbers 43, 45, 46, 47, 48, 49, 50, 51, 52, 54, 56, 58, 59, 60, 61, 62, 63, 64, 65, 66, 67, 68 and 69, Table 1_{—}7 above). Notably an over-expression of at least 3 fold was found in 25 out of 53 adenocarcinoma samples. From FIG. 27B it can be noted that expression was higher in samples from early stage patients—stages 1 and 2. From FIG. 27C it can be noted that expression is mainly higher in patients with age above 45.

Statistical analysis was applied to verify the significance of these results, as described below.

The P value for the difference in the expression levels of Homo sapiens maba1 (KIAA1324) transcripts detectable by the above amplicon in Breast cancer samples versus the normal tissue samples was determined by T test as 1.06e-003.

Threshold of 3 fold over expression was found to differentiate between cancer and normal samples with P value of 3.73e-003 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H13410_seg12F (SEQ ID NO: 235) forward primer; and H13410_seg12R (SEQ ID NO: 236) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg12 (SEQ ID NO: 237).

Expression of Homo sapiens maba1 (KIAA1324) H13410 Transcripts which are Detectable by Amplicon as Depicted in Sequence name H13410_seg12 (SEQ ID NO: 237) in Normal and Cancerous Lung Tissues

Expression of Homo sapiens maba1 (KIAA1324) transcripts detectable by or according to seg12-H13410_seg12 (SEQ ID NO: 237) amplicon and primers H13410_seg12F (SEQ ID NO: 235) and H13410_seg12R (SEQ ID NO: 236) was measured by real time PCR. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 51, 52, 53, 54, 55, 56, 57, 58, 59, 60, 61, 62, 63, 64, 69 and 70, Table 1_{—}6 above), to obtain a value of fold up-regulation for each sample relative to the median of the normal samples.

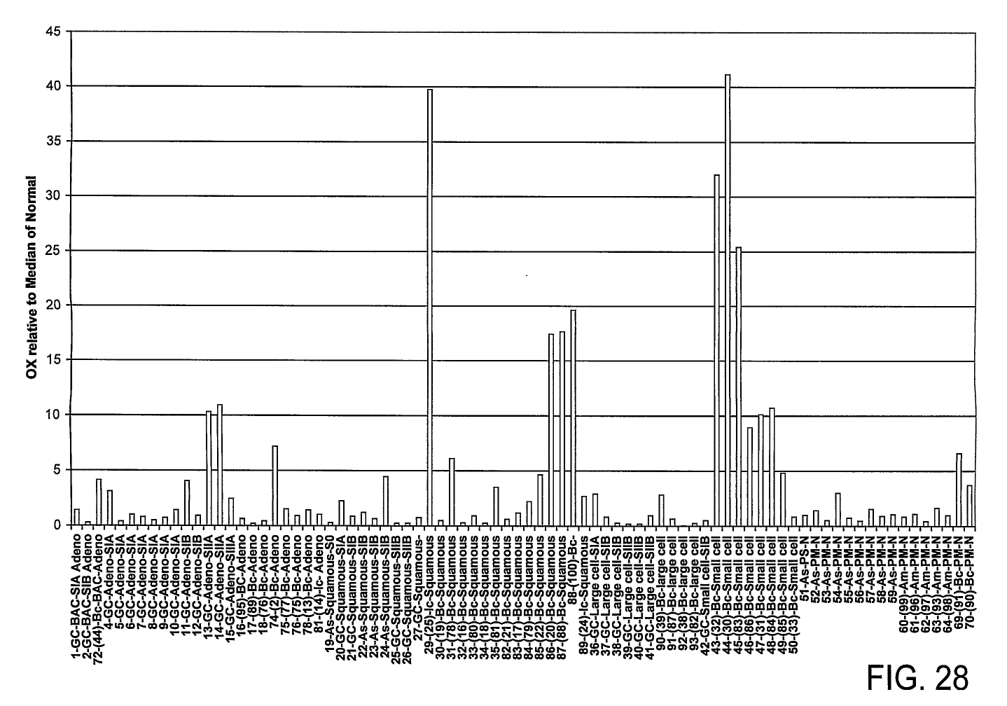

FIG. 28 is a histogram showing over expression of the above-indicated Homo sapiens maba1 (KIAA1324) transcripts in cancerous Lung samples relative to the normal samples.

As is evident from FIG. 28, the expression of Homo sapiens maba1 (KIAA1324) transcripts detectable by the above amplicon in small cell carcinoma samples was significantly higher than in the non-cancerous samples (sample numbers 51, 52, 53, 54, 55, 56, 57, 58, 59, 60, 61, 62, 63, 64, 69 and 70, Table 1_{—}6 above) and was higher in a few non-small cell carcinoma samples than in the non-cancerous samples. Notably an over-expression of at least 5 fold was found in and in 6 out of 9 small cell carcinoma samples and in 8 out of 57 non-small cell carcinoma samples −3 out of 23 adenocarcinoma samples and in 5 out of 24 squamous cell carcinoma samples.

Statistical analysis was applied to verify the significance of these results, as described below.

The P value for the difference in the expression levels of Homo sapiens maba1 (KIAA1324) transcripts detectable by the above amplicon in Lung small cell carcinoma samples versus the normal tissue samples was determined by T test as 2.44e-002.

Threshold of 5 fold over expression was found to differentiate between small cell carcinoma and normal samples with P value of 2.85e-003 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H13410_seg12F (SEQ ID NO: 235) forward primer; and H13410_seg12R (SEQ ID NO: 236) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg12 (SEQ ID NO: 237).

Expression of Homo sapiens maba1 (KIAA1324) H13410 Transcripts which are Detectable by Amplicon as Depicted in Sequence Name H13410_seg12 (SEQ ID NO: 237) in Normal and Cancerous Colon Tissues

Expression of Homo sapiens maba1 (KIAA1324) transcripts detectable by or according to seg12, H13410_seg12 (SEQ ID NO: 237) amplicon and primers H13410_seg12F (SEQ ID NO: 235) H13410_seg12R (SEQ ID NO: 236) was measured by real time PCR. In parallel the expression of three housekeeping genes—PBGD (GenBank Accession No. BC019323 (SEQ ID NO:336); amplicon—PBGD-amplicon (SEQ ID NO: 302)), HPRT1 (GenBank Accession No. NM_{—}000194 (SEQ ID NO:337); amplicon—BPRT1-amplicon (SEQ ID NO: 305) (SEQ ID NO: 305)) and G6PD (GenBank Accession No. NM_{—}000402 (SEQ ID NO:339); G6PD amplicon (SEQ ID NO: 311)) was measured similarly. For each RT sample, the expression of the above amplicon was normalized to normalization factor calculated from the expression of these house keeping genes as described in normalization method 2 in the “materials and methods” section. The normalized quantity of each RT sample was then divided by the median of the quantities of the normal samples (Sample Nos. 52, 42, 43, 44, 45, 46, 47, 48, 49, 50, 51, 53, 54, 55, 56, 57, 58, 59, 60, 61, 62, 63 and 64, Table 1_{—}8, above), to obtain a value of fold differential expression for each sample relative to the median of the normal samples.

In one experiment that was carried out no differential expression in the cancerous samples relative to the normal samples was observed.

Primer pairs are also optionally and preferably encompassed within the present invention; for example, for the above experiment, the following primer pair was used as a non-limiting illustrative example only of a suitable primer pair: H13410_seg12F (SEQ ID NO: 235) forward primer; and H13410_seg12R (SEQ ID NO: 236) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg12 (SEQ ID NO: 237).

Expression of Homo sapiens maba1 H13410 transcripts which are detectable by amplicon as depicted in sequence name H13410_seg33 (SEQ ID NO: 240) in normal and cancerous breast tissues.

Expression of Homo sapiens maba1 transcripts detectable by or according to seg33—H13410_seg33 (SEQ ID NO: 240) amplicon and primers H13410 seg33F (SEQ ID NO: 238) and H13410_seg33R (SEQ ID NO: 239) was measured by real time PCR. In parallel the expression of four housekeeping genes—G6PD (GenBank Accession No. NM_{—}000402 (SEQ ID NO:339); G6PD amplicon (SEQ ID NO: 311)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 57, 59, 60, 63, 66, 64, 56, 65, 67 and 58, Table 1_{—}3 above: “Tissue samples in breast cancer testing panel”), to obtain a value of fold up-regulation for each sample relative to the median of the normal PM samples.

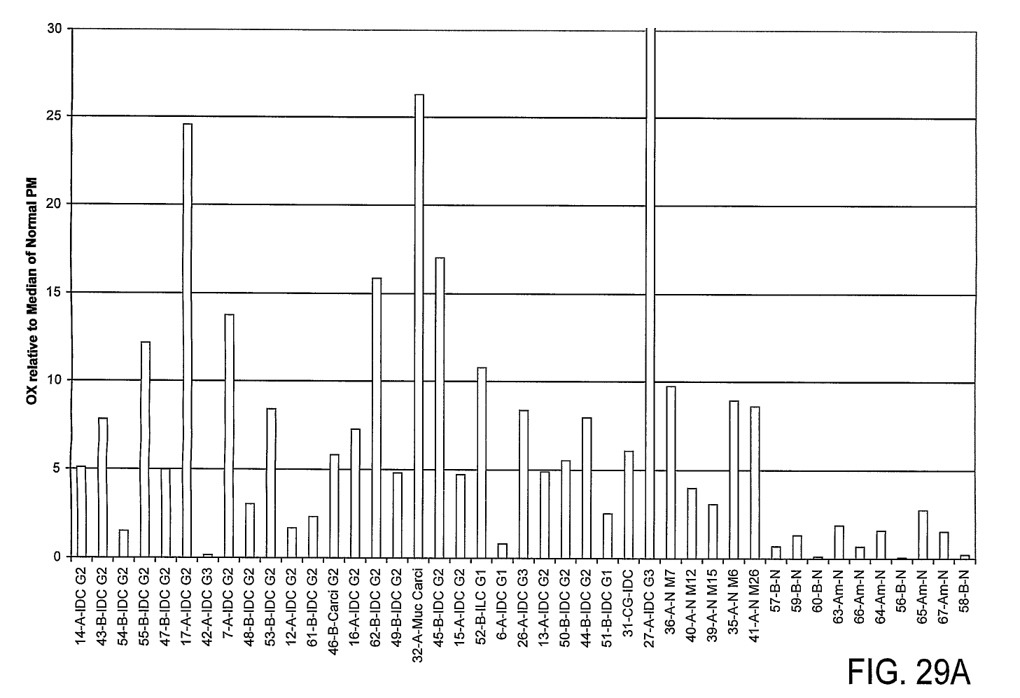

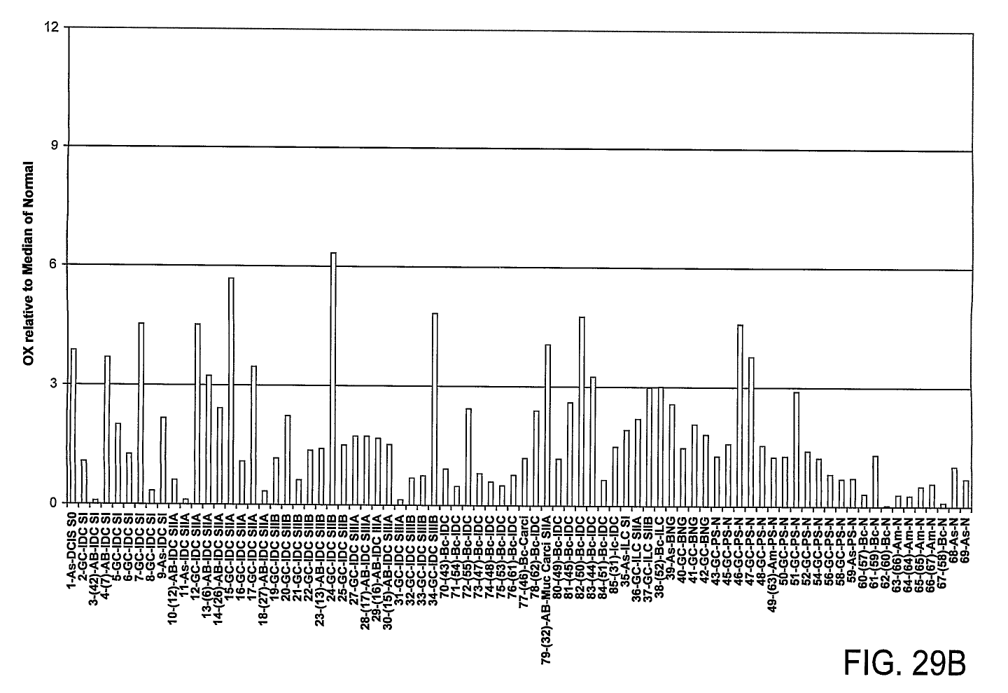

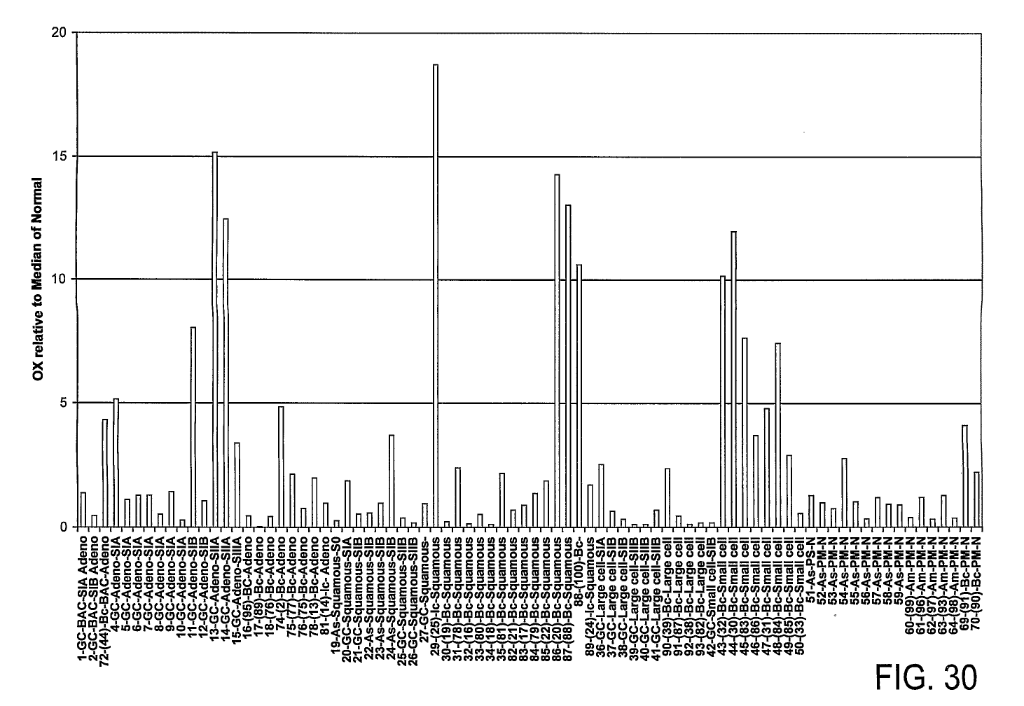

FIG. 29A is a histogram showing over expression of the Homo sapiens maba1 transcripts in cancerous Breast samples relative to the normal samples.

As is evident from FIG. 29A, the expression of Homo sapiens maba1 transcripts detectable by the above amplicon in cancer samples was significantly higher than in the non-cancerous samples (sample numbers 57, 59, 60, 63, 66, 64, 56, 65, 67 and 58, Table 1_{—}3 above, “Tissue samples in breast cancer testing panel”). Notably an over-expression of at least 5 fold was found in 17 out of 28 adenocarcinoma samples.

Statistical analysis was applied to verify the significance of these results, as described below.

The P value for the difference in the expression levels of Homo sapiens maba1 transcripts detectable by the above amplicon in Breast cancer samples versus the normal tissue samples was determined by T test as 1.54e-004.

Threshold of 5 fold over expression was found to differentiate between cancer and normal samples with P value of 7.46e-004 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H13410_seg33F (SEQ ID NO: 238) forward primer; and H13410_seg33R (SEQ ID NO: 239) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg33 (SEQ ID NO: 240).

Expression of Homo sapiens maba1 (KIAA1324) transcripts detectable by or according to seg33-H13410 seg33 (SEQ ID NO: 240) amplicon and primers H13410_seg33F (SEQ ID NO: 238) and H13410_seg33R (SEQ ID NO: 239) was further measured by real time PCR using enlarged panel as shown in Table 1_{—}7. In parallel the expression of four housekeeping genes—G6PD (GenBank Accession No. NM_{—}000402 (SEQ ID NO:339); G6PD amplicon (SEQ ID NO: 311)), RPL19 (GenBank Accession No. NM_{—}000981 (SEQ ID NO:342); RPL19 amplicon (SEQ ID NO: 320)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 43, 45, 46, 47, 48, 49, 50, 51, 52, 54, 56, 58, 59, 60, 61, 62, 63, 64, 65, 66, 67, 68 and 69, Table 1_{—}7 above), to obtain a value of fold up-regulation for each sample relative to the median of the normal samples.

FIG. 29B is a histogram showing over expression of the above-indicated Homo sapiens maba1 (KIAA1324) transcripts in cancerous Breast samples relative to the normal samples.

As is evident from FIG. 29B, the expression of Homo sapiens maba1 (KIAA1324) transcripts detectable by the above amplicon in cancer samples was higher than in the non-cancerous samples (sample numbers 43, 45, 46, 47, 48, 49, 50, 51, 52, 54, 56, 58, 59, 60, 61, 62, 63, 64, 65, 66, 67, 68 and 69, Table 1_{—}7 above). Notably an over-expression of at least 3 fold was found in 13 out of 53 adenocarcinoma samples.

Statistical analysis was applied to verify the significance of these results, as described below.

The P value for the difference in the expression levels of Homo sapiens maba1 (KIAA1324) transcripts detectable by the above amplicon in Breast cancer samples versus the normal tissue samples was determined by T test as 1.54e-002.

The above values demonstrate statistical significance of the results.

Primer pairs are also optionally and preferably encompassed within the present invention; for example, for the above experiment, the following primer pair was used as a non-limiting illustrative example only of a suitable primer pair: H13410_seg33F (SEQ ID NO: 238) forward primer; and H13410_seg33R (SEQ ID NO: 239) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg33 (SEQ ID NO: 240).

Expression of Homo sapiens maba1 (KIAA1324) H13410 Transcripts which are Detectable by Amplicon as Depicted in Sequence Name H13410_seg33 (SEQ ID NO: 240) in Normal and Cancerous Lung Tissues

Expression of Homo sapiens maba1 (KIAA1324) transcripts detectable by or according to seg33-H13410_seg33 (SEQ ID NO: 240) amplicon and primers H13410_seg33F (SEQ ID NO: 238) and H13410_seg33R (SEQ ID NO: 239) was measured by real time PCR. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 51, 52, 53, 54, 55, 56, 57, 58, 59, 60, 61, 62, 63, 64, 69 and 70, Table 1_{—}6 above), to obtain a value of fold up-regulation for each sample relative to the median of the normal samples.

FIG. 30 is a histogram showing over expression of the above-indicated Homo sapiens maba1 (KIAA1324) transcripts in cancerous Lung samples relative to the normal samples.

As is evident from FIG. 30, the expression of Homo sapiens maba1 (KIAA1324) transcripts detectable by the above amplicon in small cell carcinoma samples was significantly higher than in the non-cancerous samples (sample numbers 51, 52, 53, 54, 55, 56, 57, 58, 59, 60, 61, 62, 63, 64, 69 and 70, Table 1_{—}6 above) and was higher in a few non-small cell carcinoma samples than in the non-cancerous samples. Notably an over-expression of at least 5 fold was found in 4 out of 9 small cell carcinoma samples and in 8 out of 58 non-small cell carcinoma samples −4 out of 24 adenocarcinoma samples and in 4 out of 24 squamous cell carcinoma samples.

Statistical analysis was applied to verify the significance of these results, as described below.

The P value for the difference in the expression levels of Homo sapiens maba1 (KIAA1324) transcripts detectable by the above amplicon in Lung small cell carcinoma samples versus the normal tissue samples was determined by T test as 1.49e-002. The P value for the difference in the expression levels of Homo sapiens maba1 (KIAA1324) transcripts detectable by the above amplicon in Lung non-small cell carcinoma samples versus the normal tissue samples was determined by T test as 1.91e-002. The P value for the difference in the expression levels of Homo sapiens maba1 (KIAA1324) transcripts detectable by the above amplicon in Lung adenocarcinoma samples versus the normal tissue samples was determined by T test as 4.59e-002. The P value for the difference in the expression levels of Homo sapiens maba1 (KIAA1324) transcripts detectable by the above amplicon in Lung squamous cell carcinoma samples versus the normal tissue samples was determined by T test as 7.72e-002.

Threshold of 5 fold over expression was found to differentiate between small cell carcinoma and normal samples with P value of 9.96e-003 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H13410_seg33F (SEQ ID NO: 238) forward primer; and H13410_seg33R (SEQ ID NO: 239) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg33 (SEQ ID NO: 240).

Expression of Homo sapiens maba1 (KIAA1324) H13410 Transcripts which are Detectable by Amplicon as Depicted in Sequence Name H13410_seg33 (SEQ ID NO: 240) in Normal and Cancerous Colon Tissues

Expression of Homo sapiens maba1 (KIAA1324) transcripts detectable by or according to seg33, H13410_seg33 (SEQ ID NO: 240) amplicon and primers H13410_seg33F (SEQ ID NO: 238) H13410_seg33R (SEQ ID NO: 239) was measured by real time PCR. In parallel the expression of three housekeeping genes—PBGD (GenBank Accession No. BC019323 (SEQ ID NO:336); amplicon—PBGD-amplicon (SEQ ID NO: 302)), HPRT1 (GenBank Accession No. NM_{—}000194 (SEQ ID NO:337); amplicon—HPRT1-amplicon (SEQ ID NO: 305) (SEQ ID NO: 305)) and G6PD (GenBank Accession No. NM_{—}000402 (SEQ ID NO:339); G6PD amplicon (SEQ ID NO: 311)) was measured similarly. For each RT sample, the expression of the above amplicon was normalized to normalization factor calculated from the expression of these house keeping genes as described in normalization method 2 in the “materials and methods” section. The normalized quantity of each RT sample was then divided by the median of the quantities of the normal samples (Sample Nos. 52, 42, 43, 44, 45, 46, 47, 48, 49, 50, 51, 53, 54, 55, 56, 57, 58, 59, 60, 61, 62, 63 and 64, Table 1_{—}8, above), to obtain a value of fold differential expression for each sample relative to the median of the normal samples.

In one experiment that was carried out no differential expression in the cancerous samples relative to the normal samples was observed.

Primer pairs are also optionally and preferably encompassed within the present invention; for example, for the above experiment, the following primer pair was used as a non-limiting illustrative example only of a suitable primer pair: H13410_seg33F (SEQ ID NO: 238) forward primer; and H13410_seg33R (SEQ ID NO: 239) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg33 (SEQ ID NO: 240).

Expression of Homo sapiens Maba1 H13410 Transcripts which are Detectable by Amplicon as Depicted in Sequence Name H13410_seg58 (SEQ ID NO: 243) in Normal and Cancerous Breast Tissues.

Expression of Homo sapiens maba1 transcripts detectable by or according to seg58—H13410_seg58 (SEQ ID NO: 243) amplicon and primers H13410_seg58F (SEQ ID NO: 241) and H13410_seg58R (SEQ ID NO: 242) was measured by real time PCR. In parallel the expression of four housekeeping genes—G6PD (GenBank Accession No. NM_{—}000402 (SEQ ID NO:339); G6PD amplicon (SEQ ID NO: 311)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299) ( ) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 57, 59, 60, 63, 66, 64, 56, 65, 67 and 58, Table 1_{—}3 above: “Tissue samples in breast cancer testing panel”), to obtain a value of fold up-regulation for each sample relative to the median of the normal PM samples.

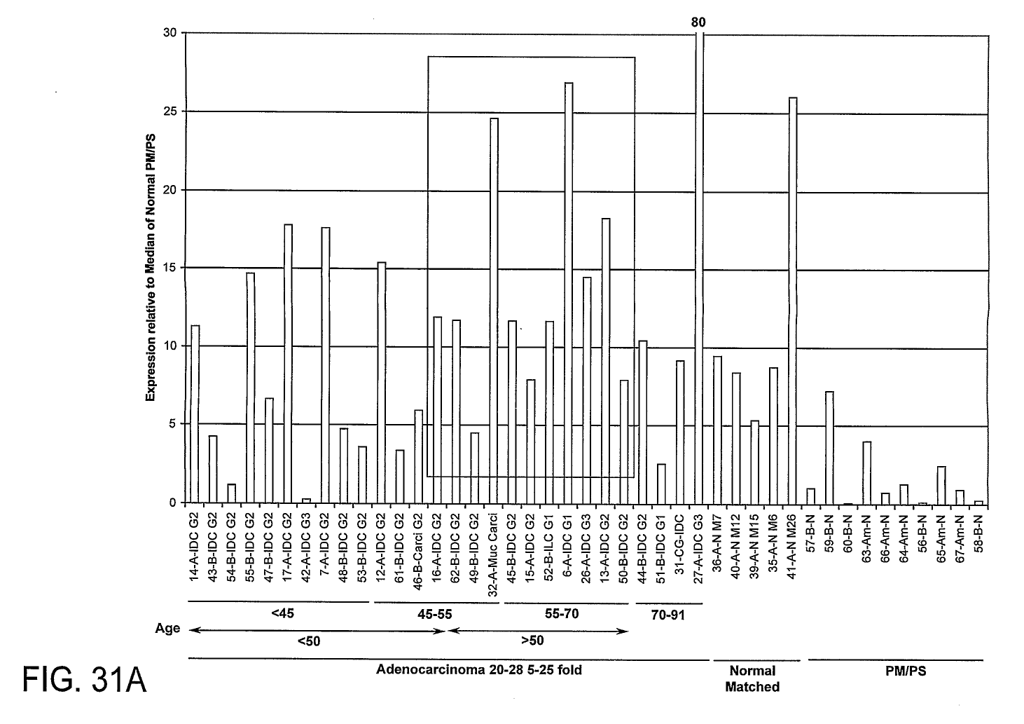

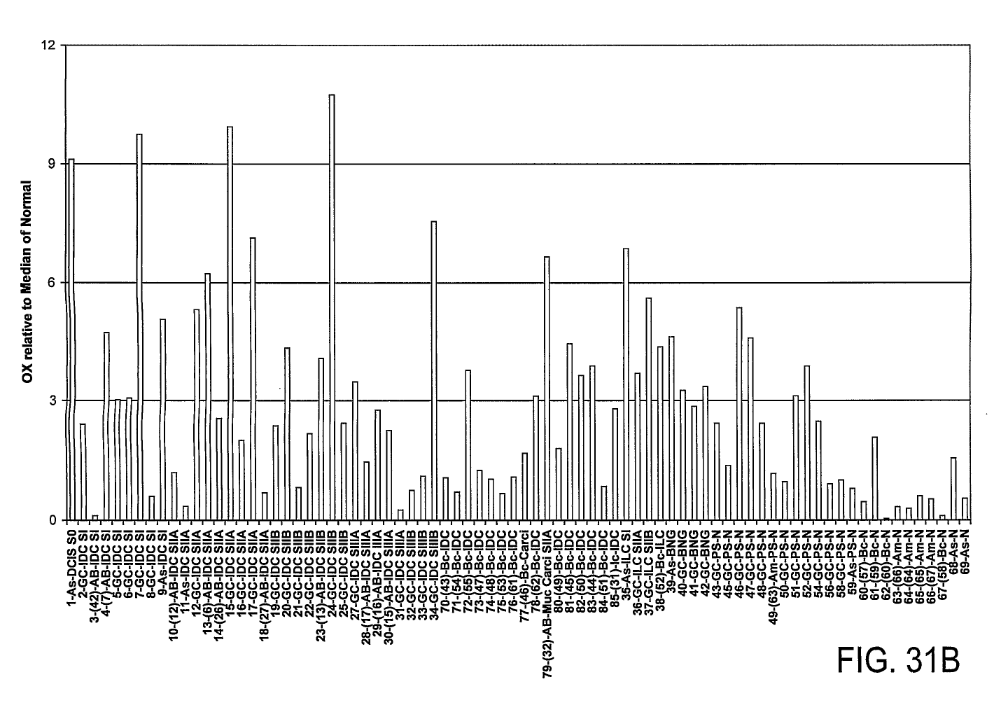

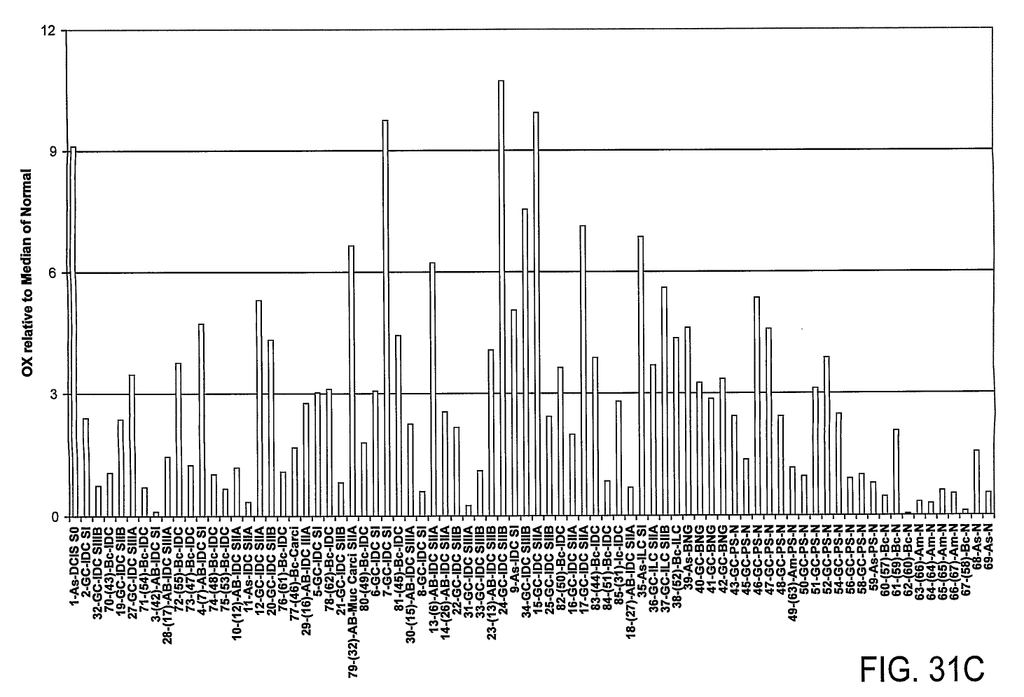

FIG. 31A is a histogram showing over expression of the Homo sapiens maba1 transcripts in cancerous Breast samples relative to the normal samples.

As is evident from FIG. 31A, the expression of Homo sapiens maba1 transcripts detectable by the above amplicon in cancer samples was significantly higher than in the non-cancerous samples (sample numbers 57, 59, 60, 63, 66, 64, 56, 65, 67 and 58, Table 1_{—}3 above: “Tissue samples in breast cancer testing panel”), especially in samples from patients with age above 50. Notably an over-expression of at least 5 fold was found in 20 out of 28 adenocarcinoma samples.

Statistical analysis was applied to verify the significance of these results, as described below.

The P value for the difference in the expression levels of Homo sapiens maba1 transcripts detectable by the above amplicon in Breast cancer samples versus the normal tissue samples was determined by T test as 6.61e-004.

Threshold of 5 fold over expression was found to differentiate between cancer and normal samples with P value of 1.12e-003 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H13410_seg58F (SEQ ID NO: 241) forward primer; and H13410_seg58R (SEQ ID NO: 242) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg58 (SEQ ID NO: 243).

Expression of Homo sapiens maba1 (KIAA1324) transcripts detectable by or according to seg58-H13410_seg58 (SEQ ID NO: 243) amplicon and primers H13410_seg58F (SEQ ID NO: 241) and H13410_seg58R (SEQ ID NO: 242) was further measured by real time PCR using enlarged panel as shown in Table 1_{—}7. In parallel the expression of four housekeeping genes—G6PD (GenBank Accession No. NM_{—}000402 (SEQ ID NO:339); G6PD amplicon (SEQ ID NO: 311)), RPL19 (GenBank Accession No. NM_{—}000981 (SEQ ID NO:342); RPL19 amplicon (SEQ ID NO: 320)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 43, 45, 46, 47, 48, 49, 50, 51, 52, 54, 56, 58, 59, 60, 61, 62, 63, 64, 65, 66, 67, 68 and 69, Table 1_{—}7 above), to obtain a value of fold up-regulation for each sample relative to the median of the normal samples.

FIGS. 31B and 31C are histograms showing over expression of the above-indicated Homo sapiens maba1 (KIAA1324) transcripts in cancerous Breast samples relative to the normal samples. In FIG. 31B—the cancer samples are arranged according to the stage of the cancer from the patient, and in FIG. 31C the samples are arranged according to patient's age.

As is evident from FIGS. 31B and 31C, the expression of Homo sapiens maba1 (KIAA1324) transcripts detectable by the above amplicon in cancer samples was significantly higher than in the non-cancerous samples (sample numbers 43, 45, 46, 47, 48, 49, 50, 51, 52, 54, 56, 58, 59, 60, 61, 62, 63, 64, 65, 66, 67, 68 and 69, Table 1_{—}7 above). Notably an over-expression of at least 3 fold was found in 25 out of 53 adenocarcinoma samples. From FIG. 31B it can be noted that expression was higher in samples from early stage patients—stages 1 and 2. From FIG. 31C it can be noted that there is some correlation between expression level and patient's age.

Statistical analysis was applied to verify the significance of these results, as described below.

The P value for the difference in the expression levels of Homo sapiens maba1 (KIAA1324) transcripts detectable by the above amplicon in Breast cancer samples versus the normal tissue samples was determined by T test as 4.76e-004.

Threshold of 3 fold over expression was found to differentiate between cancer and normal samples with P value of 1.20e-002 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H13410_seg58F (SEQ ID NO: 241) forward primer; and H13410_seg58R (SEQ ID NO: 242) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg58 (SEQ ID NO: 243).

Expression of Homo sapiens maba1 (KIAA1324) H13410 transcripts which are detectable by amplicon as depicted in sequence name H13410_seg58 (SEQ ID NO: 243) in normal and cancerous Lung tissues Expression of Homo sapiens maba1 (KIAA1324) transcripts detectable by or according to seg58-H13410_seg58 (SEQ ID NO: 243) amplicon and primers H13410_seg58F (SEQ ID NO: 241) and H13410_seg58R (SEQ ID NO: 242) was measured by real time PCR. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 51, 52, 53, 54, 55, 56, 57, 58, 59, 60, 61, 62, 63, 64, 69 and 70, Table 1_{—}6 above), to obtain a value of fold up-regulation for each sample relative to the median of the normal samples.

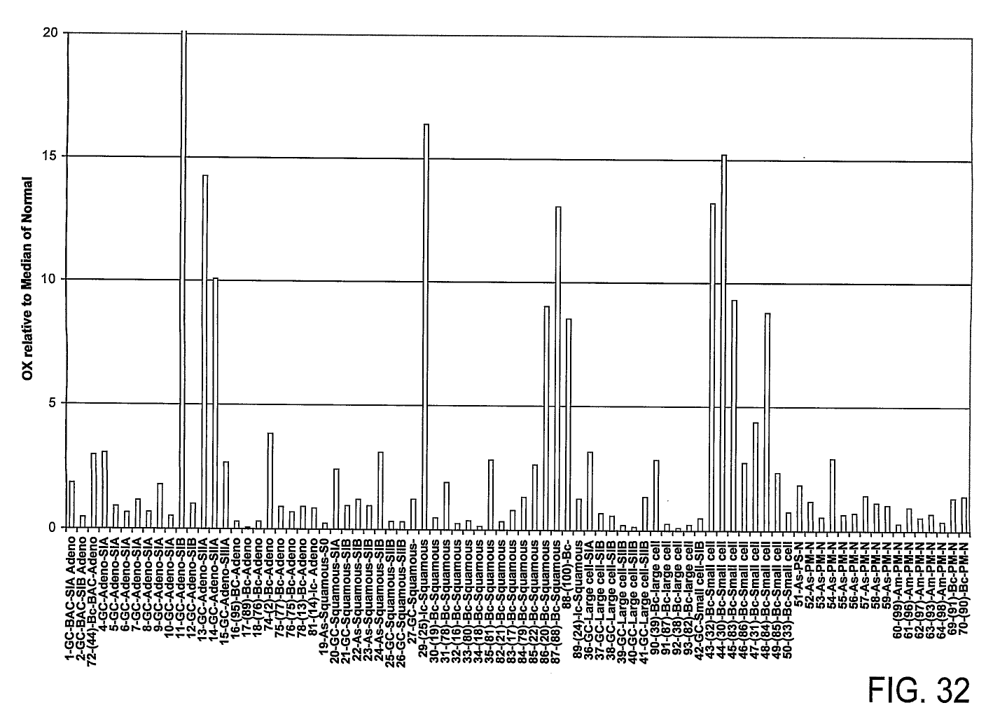

FIG. 32 is a histogram showing over expression of the above-indicated Homo sapiens maba1 (KIAA1324) transcripts in cancerous Lung samples relative to the normal samples.

As is evident from FIG. 32, the expression of Homo sapiens maba1 (KIAA1324) transcripts detectable by the above amplicon in small cell carcinoma samples was significantly higher than in the non-cancerous samples (sample numbers 51, 52, 53, 54, 55, 56, 57, 58, 59, 60, 61, 62, 63, 64, 69 and 70, Table 1_{—}6 above) and was higher in a few non-small cell carcinoma samples than in the non-cancerous samples. Notably an over-expression of at least 5 fold was found in 4 out of 9 small cell carcinoma samples, and in 7 out of 58 non-small cell carcinoma samples—3 out of 24 adenocarcinoma samples, in 4 out of 24 squamous cell carcinoma samples.

Statistical analysis was applied to verify the significance of these results, as described below.

The P value for the difference in the expression levels of Homo sapiens maba1 (KIAA1324) transcripts detectable by the above amplicon in Lung small cell carcinoma samples versus the normal tissue samples was determined by T test as 1.97e-002. The P value for the difference in the expression levels of Homo sapiens maba1 (KIAA1324) transcripts detectable by the above amplicon in Lung non-small cell carcinoma samples versus the normal tissue samples was determined by T test as 1.42e-002.

Threshold of 5 fold over expression was found to differentiate between small cell carcinoma and normal samples with P value of 9.96e-003 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H13410_seg58F (SEQ ID NO: 241) forward primer; and H13410_seg58R (SEQ ID NO: 242) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg58 (SEQ ID NO: 243).

Expression of Homo sapiens maba1 (KIAA1324) H13410 Transcripts which are Detectable by Amplicon as Depicted in Sequence Name H13410_seg58 (SEQ ID NO: 243) in Normal and Cancerous Colon Tissues

Expression of Homo sapiens maba1 (KIAA1324) transcripts detectable by or according to seg58, H13410_seg58 (SEQ ID NO: 243) amplicon and primers H13410_seg58F (SEQ ID NO: 241) H13410_seg58R (SEQ ID NO: 242) was measured by real time PCR. In parallel the expression of three housekeeping genes—PBGD (GenBank Accession No. BC019323 (SEQ ID NO:336); amplicon—PBGD-amplicon (SEQ ID NO: 302)), HPRT1 (GenBank Accession No. NM_{—}000194 (SEQ ID NO:337); amplicon—HPRT1-amplicon (SEQ ID NO: 305) (SEQ ID NO: 305)) and G6PD (Genbank Accession No. NM_{—}000402 (SEQ ID NO:339); G6PD amplicon (SEQ ID NO: 311)) was measured similarly. For each RT sample, the expression of the above amplicon was normalized to normalization factor calculated from the expression of these house keeping genes as described in normalization method 2 in the “materials and methods” section. The normalized quantity of each RT sample was then divided by the median of the quantities of the normal samples (Sample Nos. 52, 42, 43, 44, 45, 46, 47, 48, 49, 50, 51, 53, 54, 55, 56, 57, 58, 59, 60, 61, 62, 63 and 64, Table 1_{—}8, above), to obtain a value of fold differential expression for each sample relative to the median of the normal samples.

In one experiment that was carried out no differential expression in the cancerous samples relative to the normal samples was observed.

Primer pairs are also optionally and preferably encompassed within the present invention; for example, for the above experiment, the following primer pair was used as a non-limiting illustrative example only of a suitable primer pair: H13410_seg58F (SEQ ID NO: 241) forward primer; and H13410_seg58R (SEQ ID NO: 242) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg58 (SEQ ID NO: 243).

Expression of Homo sapiens maba1 H13410 Transcripts which are Detectable by Amplicon as Depicted in Sequence Name H13410_seg58 (SEQ ID NO: 243) in Different Normal Tissues.

Expression of Homo sapiens maba1 transcripts detectable by or according to seg58—H13410_seg58 (SEQ ID NO: 243) amplicon and primers H13410_seg58F (SEQ ID NO: 241) and H13410_seg58R (SEQ ID NO: 242) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the geometric mean of the quantities of the housekeeping genes. The normalized quantity of each RT sample was then divided by the median of the quantities of the breast samples (sample numbers 33, 34 and 35, Table 1_{—}9 above: “Tissue samples in normal panel”), to obtain a value of relative expression of each sample relative to the median of the breast samples.

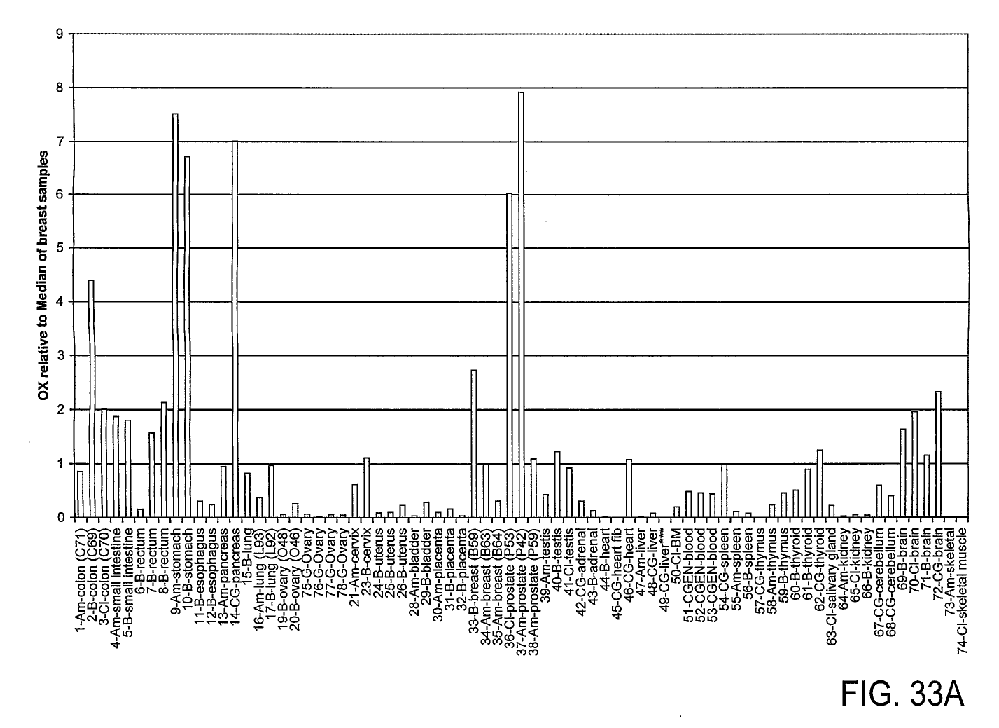

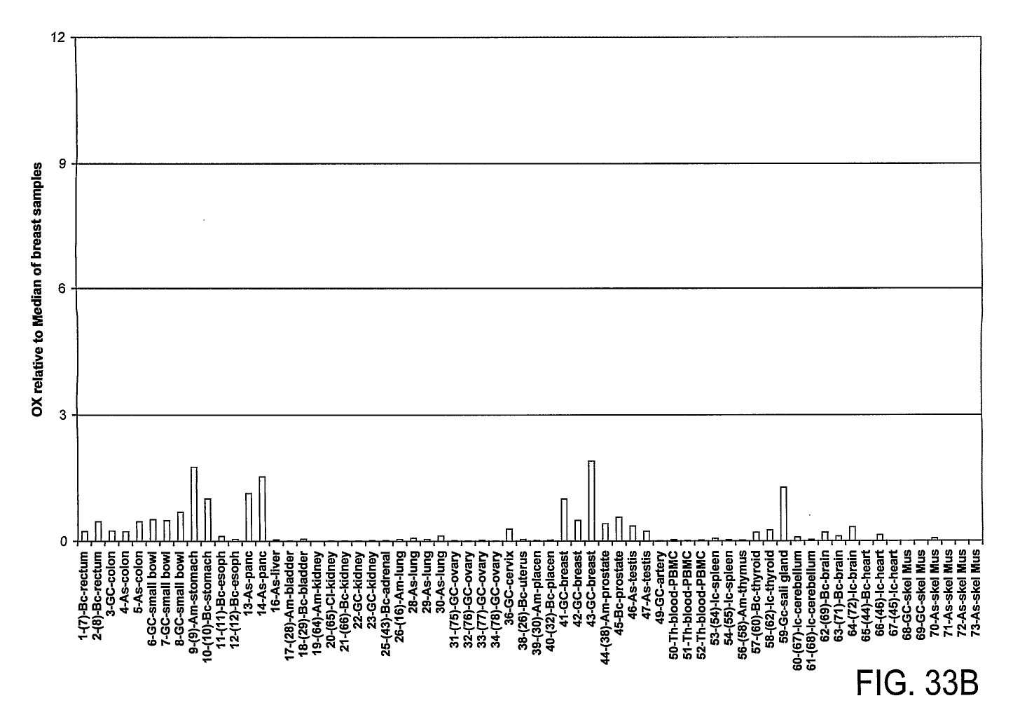

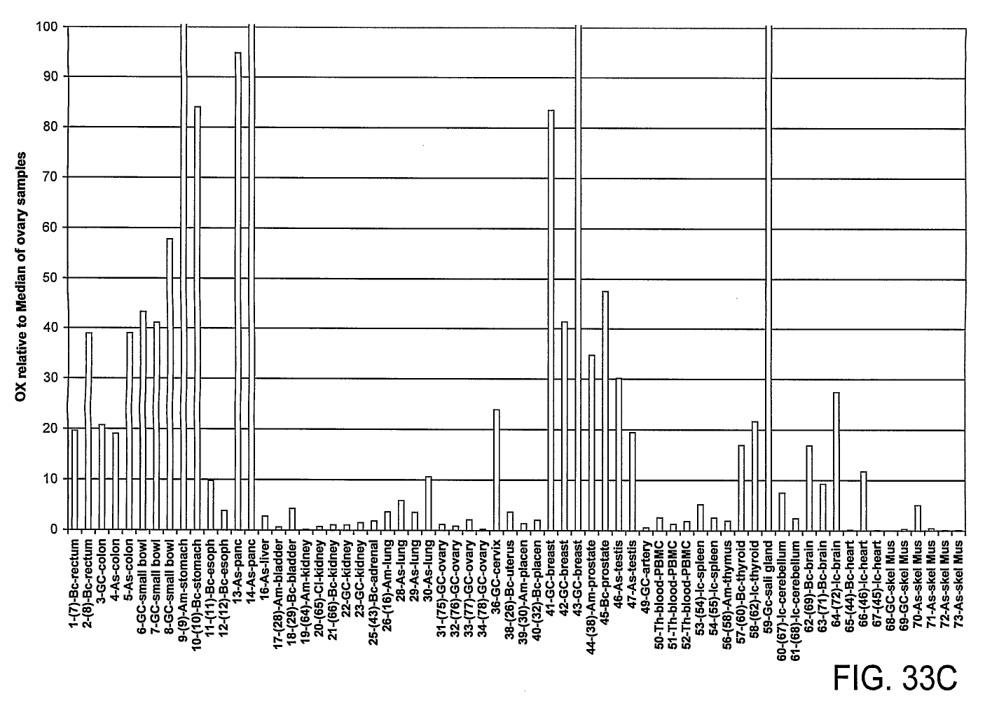

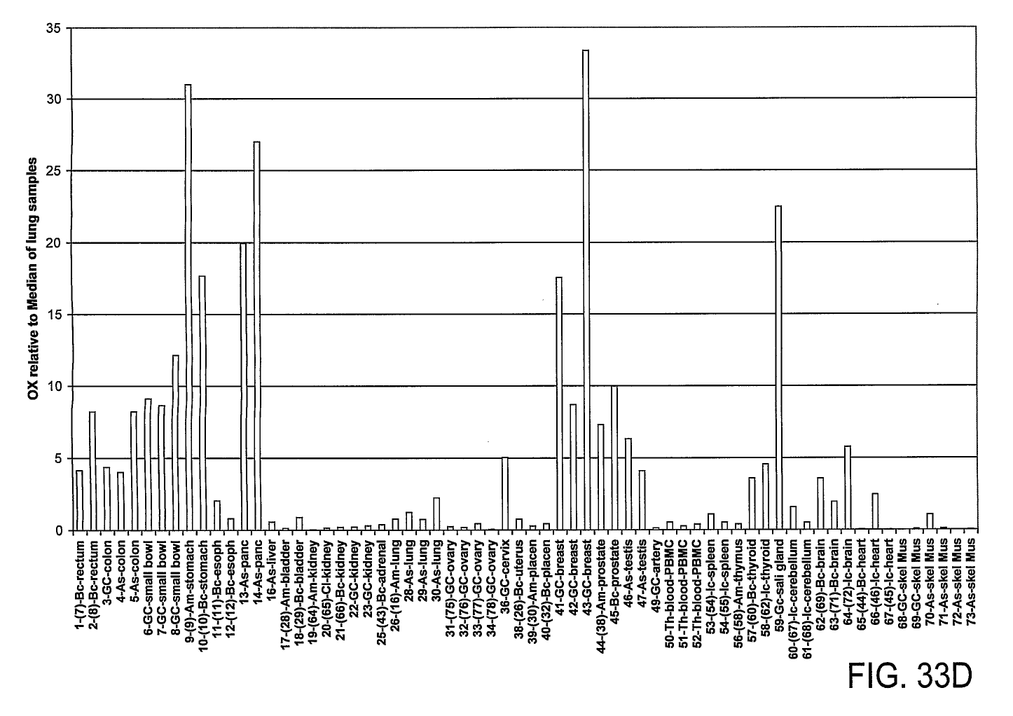

FIG. 33A is a histogram showing the Expression of Homo sapiens maba1 H13410 transcripts which are detectable by amplicon as depicted in sequence name H13410_seg58 (SEQ ID NO: 243) in different normal tissues.

Expression of Homo sapiens maba1 (KIAA1324) transcripts detectable by or according to seg58-H13410_seg58 (SEQ ID NO: 243) amplicon and primers H13410_seg58F (SEQ ID NO: 241) and H13410_seg58R (SEQ ID NO: 242) was further measured by real time PCR using updated panel, as shown in Table 1_{—}10. In parallel the expression of three housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the breast samples (sample numbers 41, 42 and 43, Table 1_{—}10 above), to obtain a value of relative expression of each sample relative to the median of the breast samples—as presented FIG. 33B, or to the median of the quantities of the ovary samples (sample numbers 31, 32, 33 and 34, Table 1_{—}10 above), to obtain a value of relative expression of each sample relative to the median of the ovary samples—as presented in FIG. 33C, or to the median of the quantities of the lung samples (sample numbers 28, 29 and 30, Table 1_{—}10 above), to obtain a value of relative expression of each sample relative to the median of the lung samples—as presented in FIG. 33D.

Expression of Homo sapiens maba1 H13410 Transcripts which are Detectable by Amplicon as Depicted in Sequence Name H13410_seg12 (SEQ ID NO: 237) in Different Normal Tissues.

Expression of Homo sapiens maba1 transcripts detectable by or according to seg12—H13410_seg12 (SEQ ID NO: 237) amplicon and primers H13410_seg12F (SEQ ID NO: 235) and H13410_seg12R (SEQ ID NO: 236) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the geometric mean of the quantities of the housekeeping genes. The normalized quantity of each RT sample was then divided by the median of the quantities of the breast samples (sample numbers 33, 34 and 35, Table 119 above: “Tissue samples in normal panel”), to obtain a value of relative expression of each sample relative to the median of the breast samples.

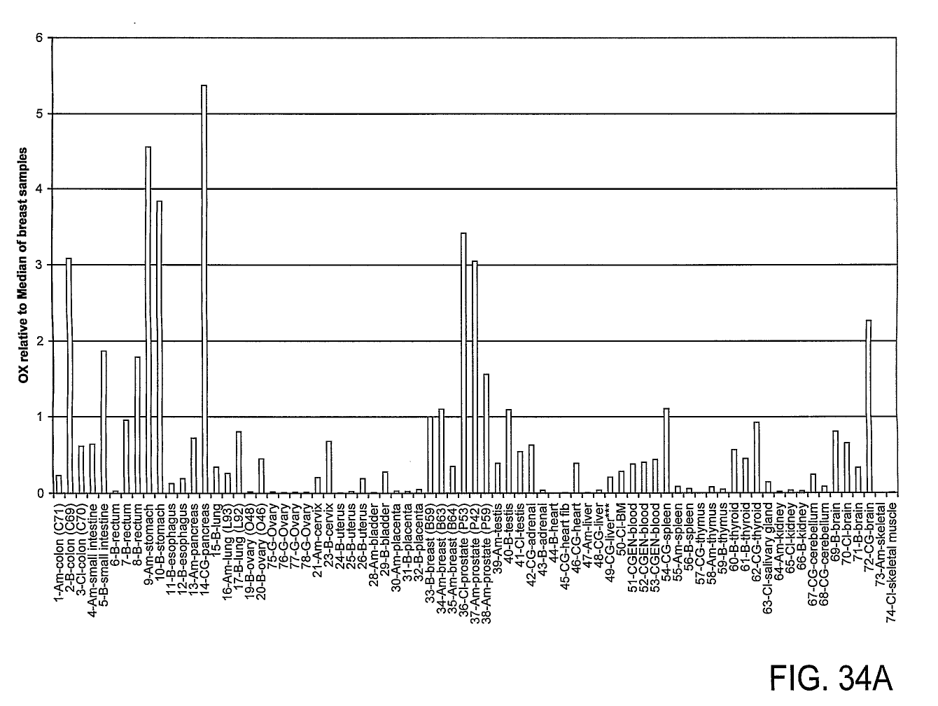

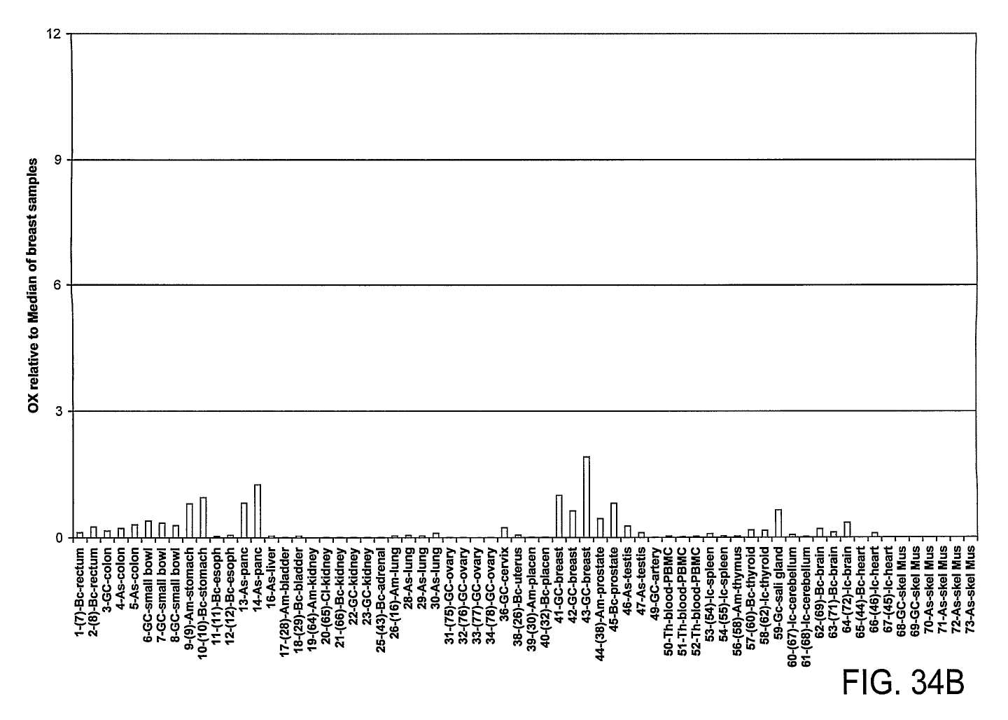

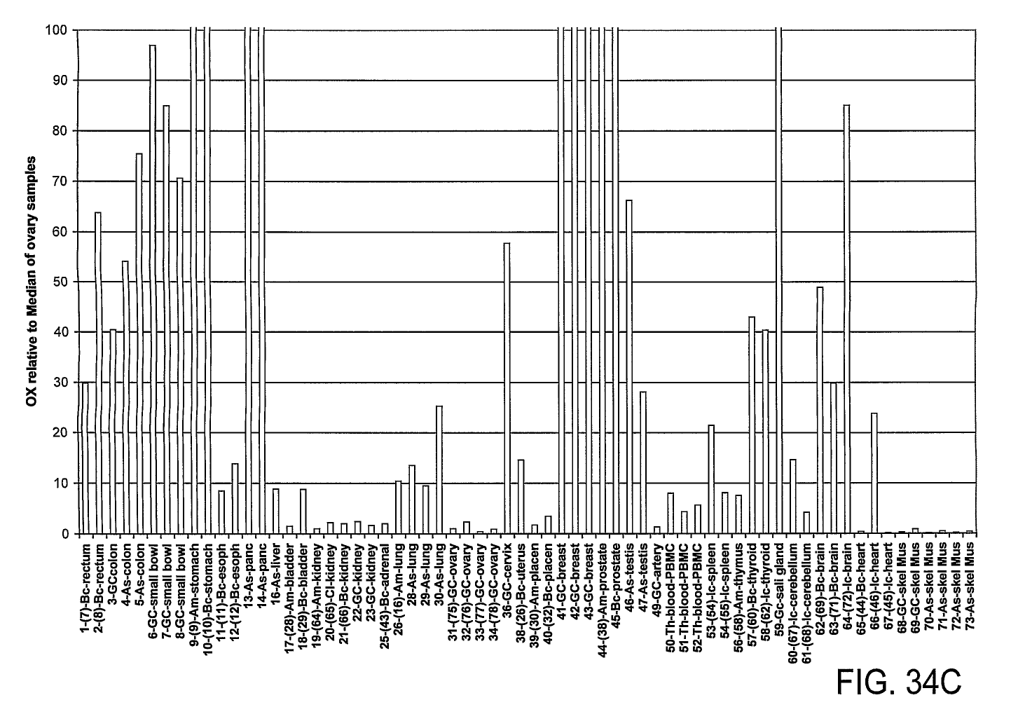

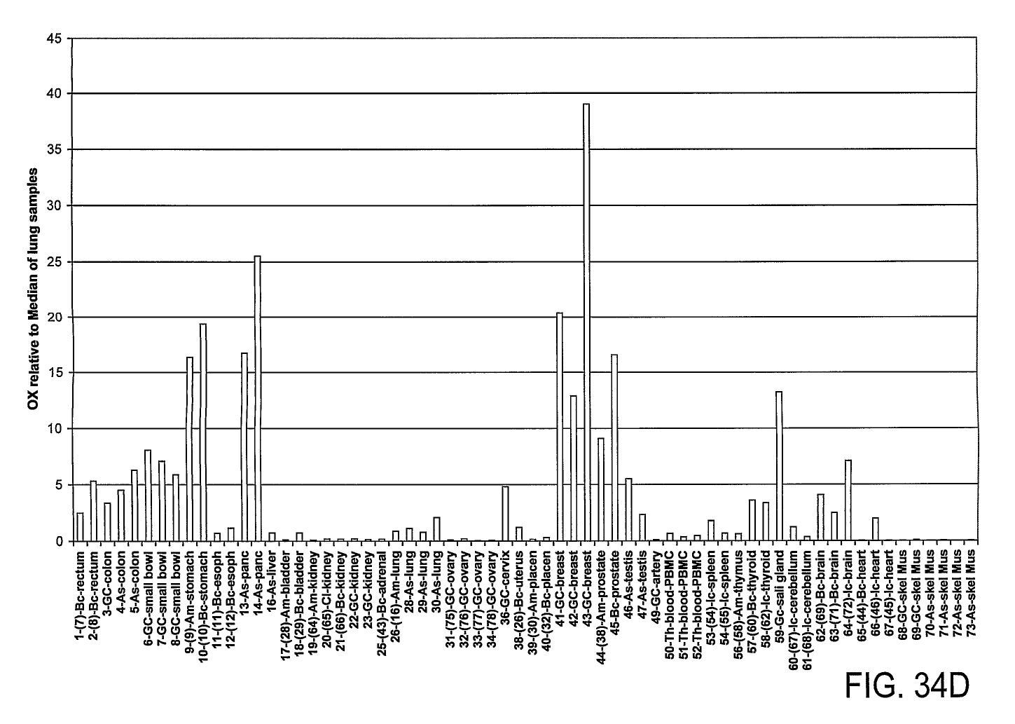

FIG. 34A is a histogram showing the expression of Homo sapiens maba1 H13410 transcripts which are detectable by amplicon as depicted in sequence name H13410_seg12 (SEQ ID NO: 237) in different normal tissues.

Expression of Homo sapiens maba1 (KIAA1324) transcripts detectable by or according to seg12-H13410_seg12 (SEQ ID NO: 237) amplicon and primers H13410_seg12F (SEQ ID NO: 235) and H13410_seg12R (SEQ ID NO: 236) was further measured by real time PCR using updated panel, as presented in table 1_{—}10. In parallel the expression of three housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), and TATA box (GenBank Accession No. NM_{—}003194 (SEQ ID NO:343); TATA amplicon (SEQ ID NO: 323)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the breast samples (sample numbers 41, 42 and 43, Table 1_{—}10 above), to obtain a value of relative expression of each sample relative to the median of the breast samples—as presented in FIG. 34B. The normalized the median of the quantities of the ovary samples (sample numbers 31, 32, 33 and 34, Table 1_{—}10 above), to obtain a value of relative expression of each sample relative to the median of the ovary samples—as presented in FIG. 34C, or by the median of the quantities of the lung samples (sample numbers 28, 29 and 30, Table 1_{—}10 above), to obtain a value of relative expression of each sample relative to the median of the lung samples—as presented in FIG. 34D.

FIG. 34 is a histogram showing expression of Homo sapiens maba1 (KIAA1324) H13410 transcripts which are detectable by amplicon as depicted in sequence name H13410_seg12 (SEQ ID NO: 237) in different normal tissues.

Expression of Homo sapiens maba1 H13410 Transcripts which are Detectable by Amplicon as Depicted in Sequence Name H13410_seg33 (SEQ ID NO: 240) in Different Normal Tissues

Expression of Homo sapiens maba1 transcripts detectable by or according to seg33—H13410_seg33 (SEQ ID NO: 240) amplicon and primers H13410_seg33F (SEQ ID NO: 238) and H13410_seg33R (SEQ ID NO: 239) was measured by real time PCR. In parallel the expression of four housekeeping genes—SDHA (GenBank Accession No. NM 004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the geometric mean of the quantities of the housekeeping genes. The normalized quantity of each RT sample was then divided by the median of the quantities of the breast samples (sample numbers 33, 34 and 35, Table 1_{—}9 above), to obtain a value of relative expression of each sample relative to the median of the breast samples.

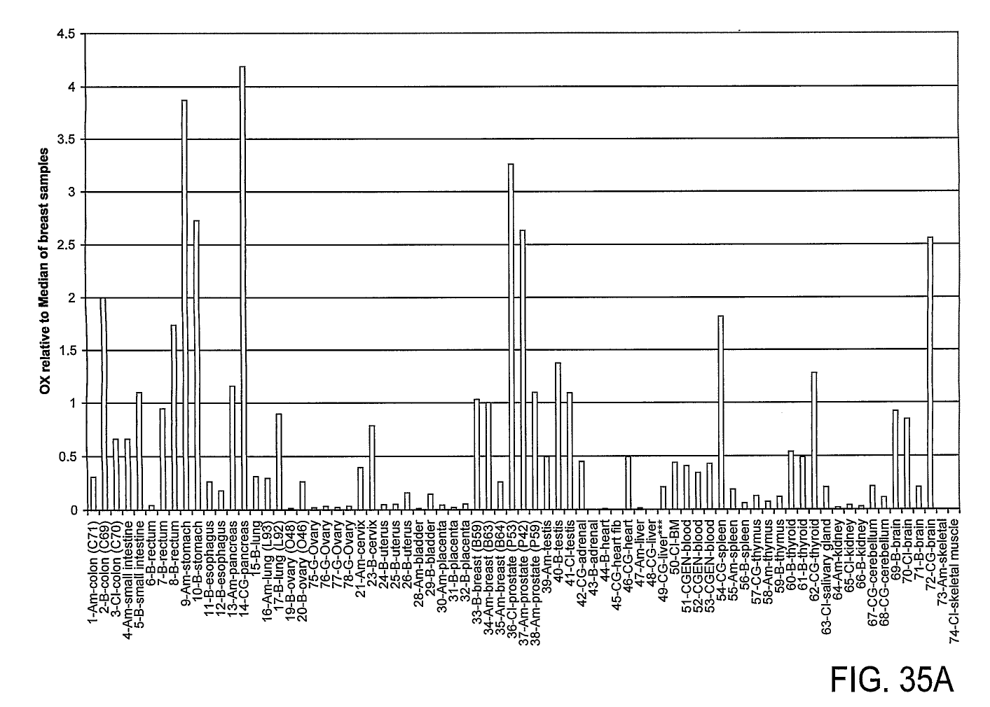

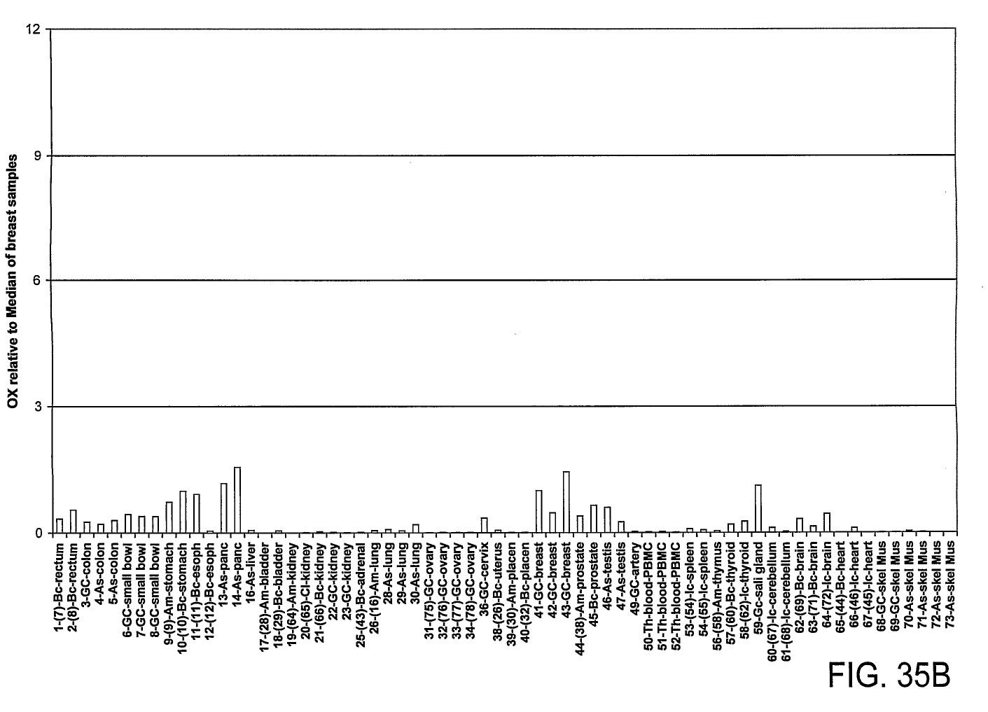

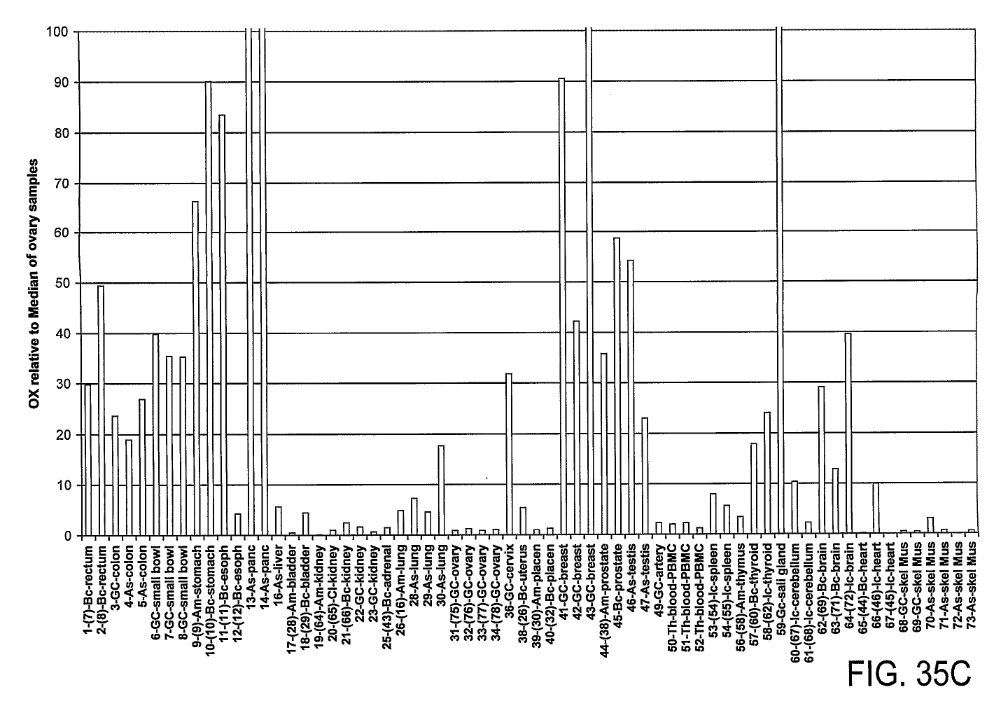

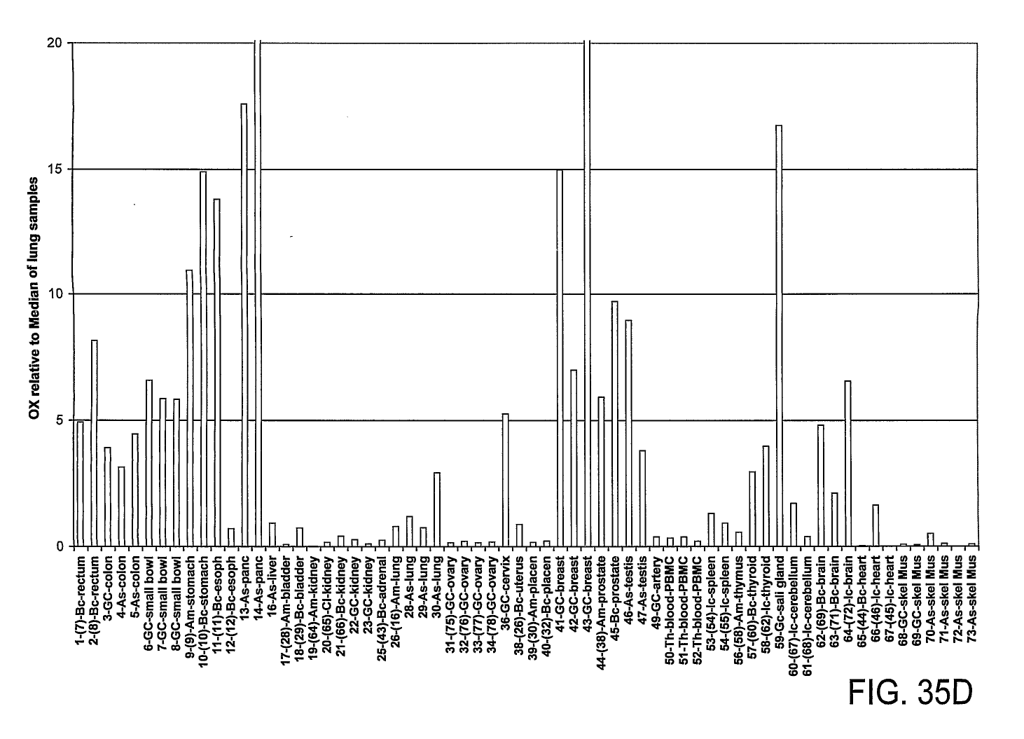

FIG. 35A is a histogram showing the expression of Homo sapiens maba1 H13410 transcripts which are detectable by amplicon as depicted in sequence name H13410_seg33 (SEQ ID NO: 240) in different normal tissues.

Expression of Homo sapiens maba1 (KIAA1324) transcripts detectable by or according to seg33-H13410_seg33 (SEQ ID NO: 240) amplicon and primers H13410_seg33F (SEQ ID NO: 238) and H13410_seg33R (SEQ ID NO: 239) was further measured by real time PCR on updated panel, as shown in Table 1_{—}10. In parallel the expression of three housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), and TATA box (GenBank Accession No. NM_{—}003194 (SEQ ID NO:343); TATA amplicon (SEQ ID NO: 323)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the breast samples (sample numbers 41, 42 and 43, Table 1_{—}10 above), to obtain a value of relative expression of each sample relative to the median of the breast samples—as presented in FIG. 35B, or to the median of the quantities of the ovary samples (sample numbers 31, 32, 33 and 34, Table 1_{—}10 above), to obtain a value of relative expression of each sample relative to the median of the ovary samples—as presented in FIG. 35C, or to the median of the quantities of the lung samples (sample numbers 28, 29 and 30, Table 1_{—}10 above), to obtain a value of relative expression of each sample relative to the median of the lung samples—see FIG. 35D.

Expression of Homo sapiens maba1 (KIAA1324) H13410 Transcripts which are Detectable by Amplicon as Depicted in Sequence Name H13410_seg12 (SEQ ID NO: 237) in Normal and Cancerous Ovary Tissues

Expression of Homo sapiens maba1 (KIAA1324) transcripts detectable by or according to seg12-H13410_seg12 (SEQ ID NO: 237) amplicon and primers H13410_seg12F (SEQ ID NO: 235) and H13410_seg12R (SEQ ID NO: 236) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and GAPDH (GenBank Accession No. BC026907 (SEQ ID NO:338); GAPDH amplicon (SEQ ID NO: 308)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 45, 46, 71 and 48, Table 1_{—}1 above), to obtain a value of fold up-regulation for each sample relative to the median of the normal PM samples.

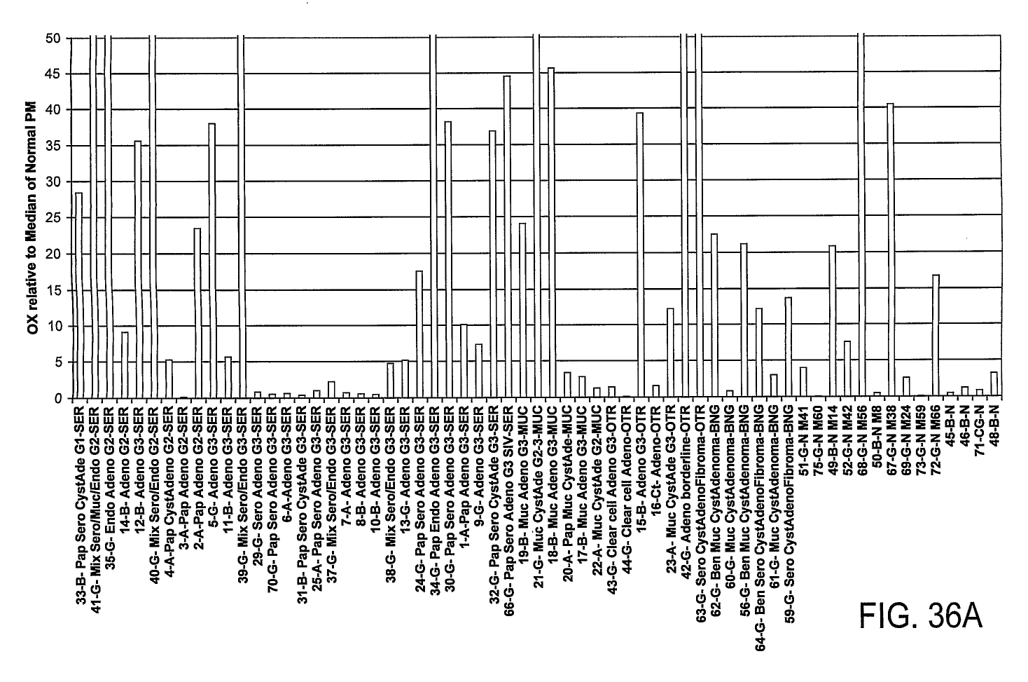

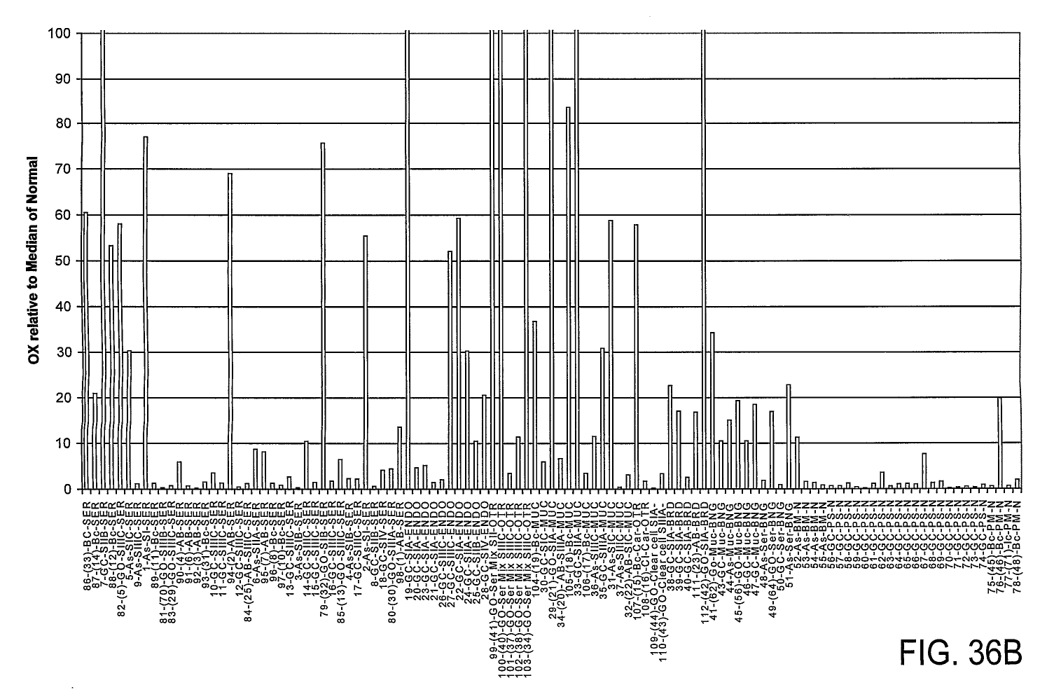

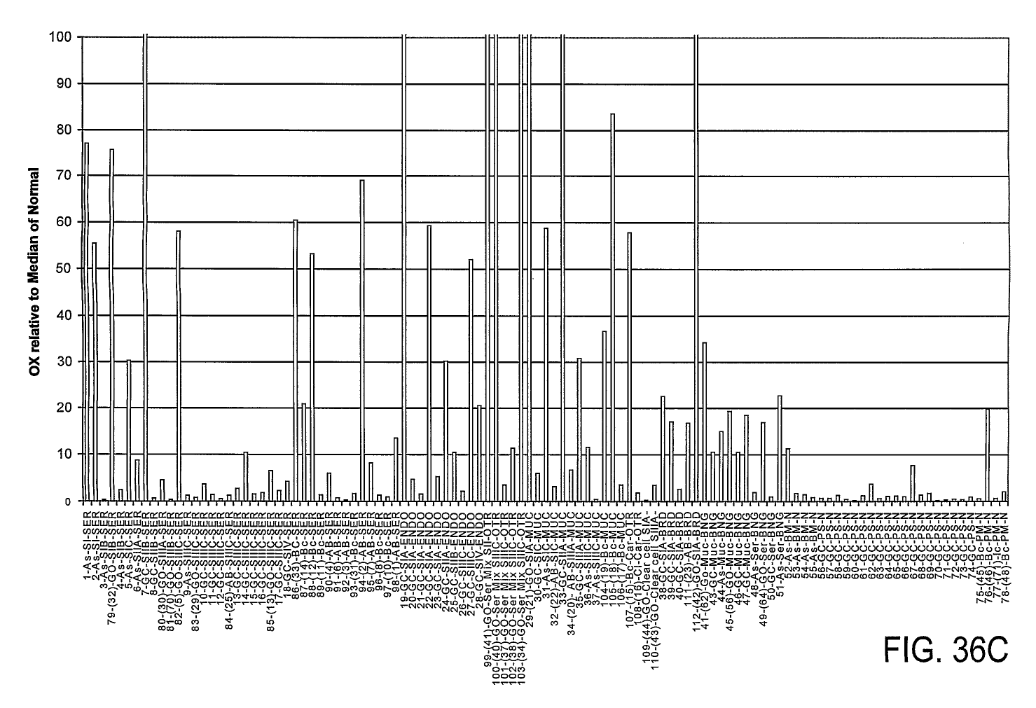

FIG. 36A is a histogram showing over expression of the Homo sapiens maba1 (KIAA1324) transcripts in cancerous Ovary samples relative to the normal samples.

As is evident from FIG. 36A, the expression of Homo sapiens maba1 (KIAA1324) transcripts detectable by the above amplicon in adenocarcinoma samples was significantly higher than in the non-cancerous samples (sample numbers 45, 46, 71 and 48, Table 1_{—}1 above). Notably an over-expression of at least 5 fold was found in 26 out of 43 adenocarcinoma samples, specifically in 19 out of 30 serous carcinoma samples and in 3 out of 6 mucinous carcinoma samples. Over expression was observed daily in patients with age lower than 50 or higher than 68.

Statistical analysis was applied to verify the significance of these results, as described below.

The P value for the difference in the expression levels of Homo sapiens maba1 (KIAA1324) transcripts detectable by the above amplicon in Ovary serous carcinoma samples versus the normal tissue samples was determined by T test as 2.61e-003. The P value for the difference in the expression levels of Homo sapiens maba1 (KIAA1324) transcripts detectable by the above amplicon in all ovary adenocarcinoma samples versus the normal tissue samples was determined by T test as 1.74e-004.

Threshold of 5 fold over expression was found to differentiate between serous carcinoma and normal samples with P value of 2.94e-002 as checked by exact Fisher test. Threshold of 5 fold over expression was found to differentiate between all adenocarcinoma and normal samples with P value of 3.36e-002 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H13410_seg12F (SEQ ID NO: 235) forward primer; and H13410_seg12R (SEQ ID NO: 236) reverse primer

The present invention also preferably encompasses any amplicon obtained through the use of any suitable primer pair; for example, for the above experiment, the following amplicon was obtained as a non-limiting illustrative example only of a suitable amplicon: H13410_seg12 (SEQ ID NO: 237).

Expression of Homo sapiens maba1 (KIAA1324) transcripts detectable by or according to seg12-H13410_seg12 (SEQ ID NO: 237) amplicon and primers H13410_seg12F (SEQ ID NO: 235) and H13410_seg12R (SEQ ID NO: 236) was further measured by real time PCR using enlarged panel as shown in Table 1-5. In parallel the expression of three housekeeping genes—SDHA (GenBank Accession No. NM_{—}004168 (SEQ ID NO:335); amplicon—SDHA-amplicon (SEQ ID NO:299)), HPRT1 (GenBank Accession No. NM 000194 (SEQ ID NO:337); amplicon—HPRT1-amplicon (SEQ ID NO: 305) (SEQ ID NO: 305)) and G6PD (GenBank Accession No. NM_{—}000402 (SEQ ID NO:339); G6PD amplicon (SEQ ID NO: 311)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above), to obtain a value of fold up-regulation for each sample relative to the median of the normal samples.

FIGS. 36B and 36C are histograms showing over expression of the above-indicated Homo sapiens maba1 (KIAA1324) transcripts in cancerous Ovary samples relative to the normal samples. In FIG. 36B the samples are arranged according to the patient's age, and in FIG. 36C samples are arranged according to patient's stage.

As is evident from FIGS. 36B and 36C, the expression of Homo sapiens maba1 (KIAA1324) transcripts detectable by the above amplicon in adenocarcinoma samples was significantly higher than in the non-cancerous samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above). Notably an over-expression of at least 5 fold was found in 37 out if 69 adenocarcinoma samples, specifically in 20 out of 43 serous carcinoma samples, in 9 out of 12 mucinous carcinoma samples and in 7 out of 10 endometroid samples. From FIG. 36B it can be observed that in serous samples over expression was mainly in patients with age below 50, in mucinus samples over expression was higher in patients with age below 50 and in endometroid over expression was mainly in patients with age above 50. From FIG. 36C it can be seen that in serous samples over expression was mainly in patients with stage I and II.

Statistical analysis was applied to verify the significance of these results, as described below.

The P value for the difference in the expression levels of Homo sapiens maba1 (KIAA1324) transcripts detectable by the above amplicon in Ovary adenocarcinoma samples versus the normal tissue samples was determined by T test as 1.96e-005. The P value for the difference in the expression levels of Homo sapiens maba1 (KIAA1324) transcripts detectable by the above amplicon in Ovary serous carcinoma samples versus the normal tissue samples was determined by T test as 1.29e-003. The P value for the difference in the expression levels of Homo sapiens maba1 (KIAA1324) transcripts detectable by the above amplicon in Ovary mucinous carcinoma samples versus the normal tissue samples was determined by T test as 5.06e-002. The P value for the difference in the expression levels of Homo sapiens maba1 (KIAA1324) transcripts detectable by the above amplicon in Ovary endometroid samples versus the normal tissue samples was determined by T test as 4.05e-002.

Threshold of 5 fold over expression was found to differentiate between adenocarcinoma and normal samples with P value of 8.92e-005 as checked by exact Fisher test. Threshold of 5 fold over expression was found to differentiate between serous carcinoma and normal samples with P value of 1.78e-003 as checked by exact Fisher test. Threshold of 5 fold over expression was found to differentiate between mucinous carcinoma and normal samples with P value of 1.69e-004 as checked by exact Fisher test. Threshold of 5 fold over expression was found to differentiate between endometroid and normal samples with P value of 1.04e-003 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H13410_seg12F (SEQ ID NO: 235) forward primer; and H13410_seg12R (SEQ ID NO: 236) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg12 (SEQ ID NO: 237).

Expression of Homo sapiens maba1 (KIAA1324) H113410 Transcripts which are Detectable by Amplicon as Depicted in Sequence Name H13410_seg33 (SEQ ID NO: 240) in Normal and Cancerous Ovary Tissues

Expression of Homo sapiens maba1 (KIAA1324) transcripts detectable by or according to seg33-H13410_seg33 (SEQ ID NO: 240) amplicon and primers H13410_seg33F (SEQ ID NO: 238) and H13410_seg33R (SEQ ID NO: 239) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and GAPDH (GenBank Accession No. BC026907 (SEQ ID NO:338); GAPDH amplicon (SEQ ID NO: 308)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 45, 46, 71 and 48, Table 1_{—}1 above), to obtain a value of fold up-regulation for each sample relative to the median of the normal PM samples.

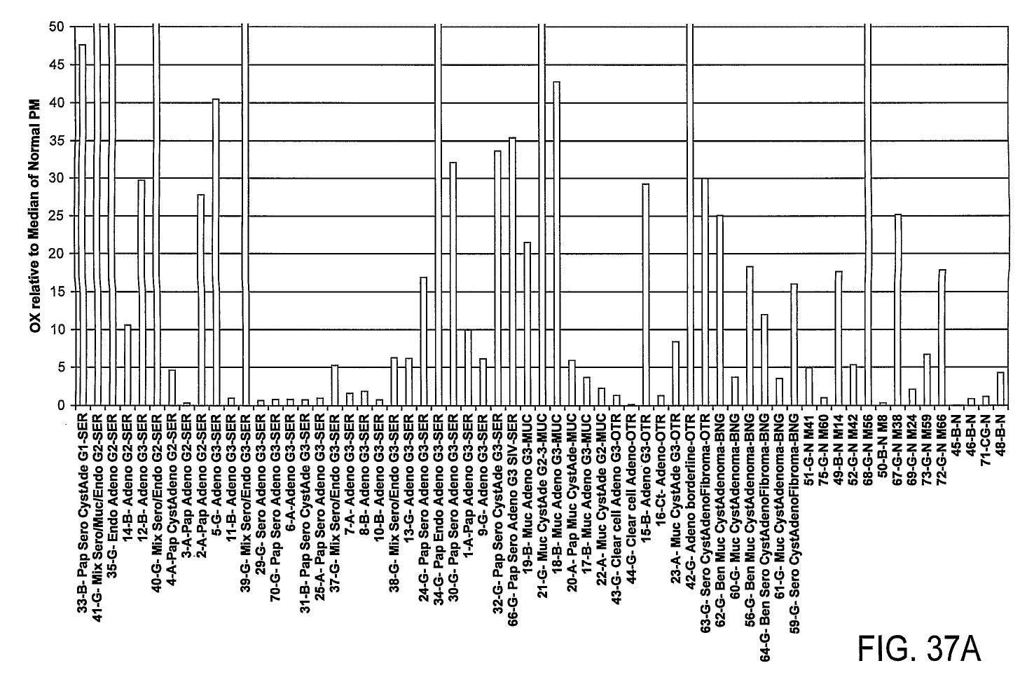

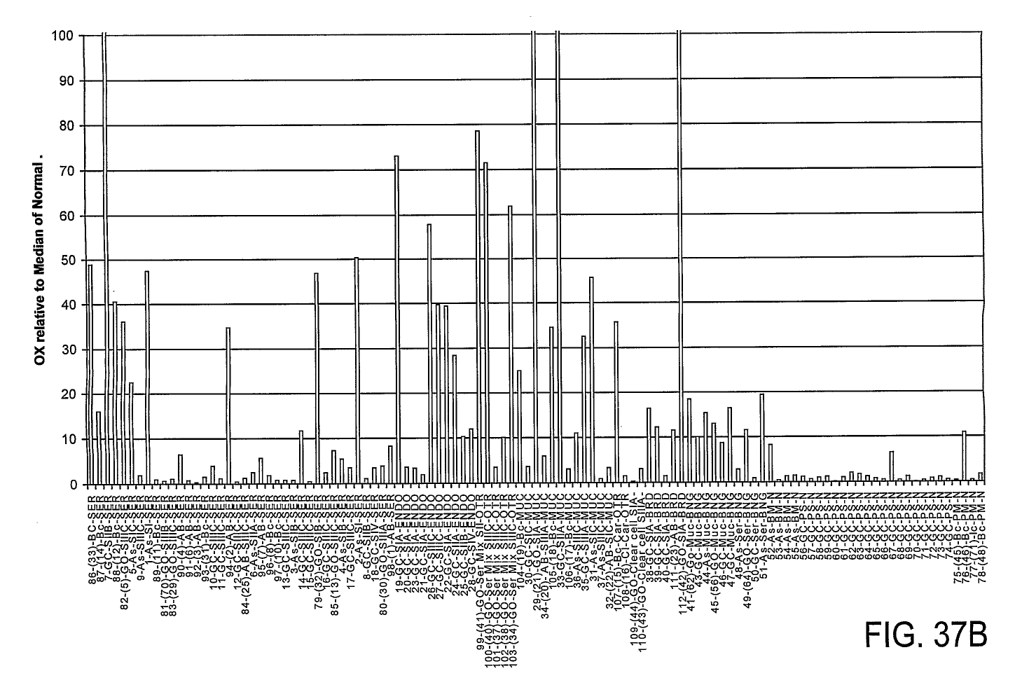

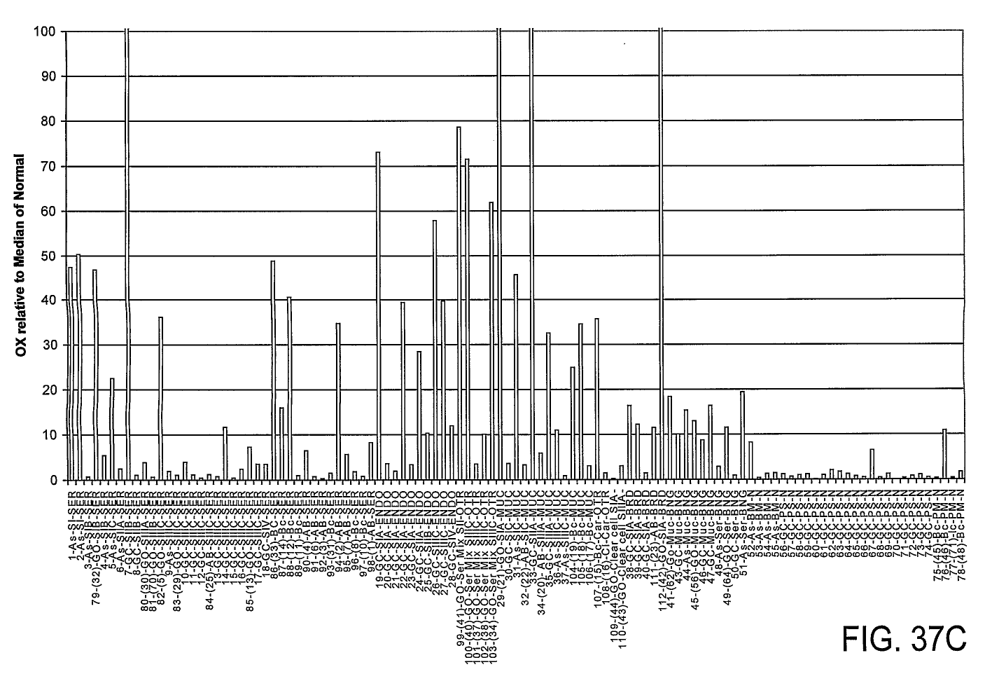

FIG. 37A is a histogram showing over expression of the Homo sapiens maba1 (KIAA1324) transcripts in cancerous Ovary samples relative to the normal samples.

As is evident from FIG. 37A, the expression of Homo sapiens maba1 (KIAA1324) transcripts detectable by the above amplicon in adenocarcinoma samples was significantly higher than in the non-cancerous samples (sample numbers 45, 46, 71 and 48, Table 1_{—}1 above). Notably an over-expression of at least 5 fold was found in 27 out of 43 adenocarcinoma samples, specifically in 19 out of 30 serous carcinoma samples and in 4 out of 6 mucinous carcinoma samples. Over expression was observed maily in patients with age lower than 50 or higher than 68.

Statistical analysis was applied to verify the significance of these results, as described below.

The P value for the difference in the expression levels of Homo sapiens maba1 (KIAA1324) transcripts detectable by the above amplicon in ovary serous carcinoma samples versus the normal tissue samples was determined by T test as 1.23e-003. The P value for the difference in the expression levels of Homo sapiens maba1 (KIAA1324) transcripts detectable by the above amplicon in all ovary adenocarcinoma samples versus the normal tissue samples was determined by T test as 7.47e-005.

Threshold of 5 fold over expression was found to differentiate between serous carcinoma and normal samples with P value of 2.94e-002 as checked by exact Fisher test. Threshold of 5 fold over expression was found to differentiate between all adenocarcinoma and normal samples with P value of 2.72e-002 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H13410_seg33F (SEQ ID NO: 238) forward primer; and H13410_seg33R (SEQ ID NO: 239) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg33 (SEQ ID NO: 240).

Expression of Homo sapiens maba1 (KIAA1324) transcripts detectable by or according to seg33-H13410_seg33 (SEQ ID NO: 240) amplicon and primers H13410_seg33F (SEQ ID NO: 238) and H13410_seg33R (SEQ ID NO: 239) was further measured by real time PCR using enlarged panel as shown in Table 1_{—}5. In parallel the expression of three housekeeping genes—SDHA (GenBank Accession No. NM_{—}004168 (SEQ ID NO:335); amplicon—SDHA-amplicon (SEQ ID NO:299)), EPRT1 (GenBank Accession No. NM_{—}000194 (SEQ ID NO:337); amplicon—HPRT1-amplicon (SEQ ID NO: 305) (SEQ ID NO: 305)) and G6PD (GenBank Accession No. NM_{—}000402 (SEQ ID NO:339); G6PD amplicon (SEQ ID NO: 311)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above), to obtain a value of fold up-regulation for each sample relative to the median of the normal samples.

FIGS. 37B and 37C are histograms showing over expression of the above-indicated Homo sapiens maba1 (KIAA1324) transcripts in cancerous Ovary samples relative to the normal samples. In FIG. 37B the samples are arranged according to the patient's age, and in FIG. 37C samples are arranged according to the patient's stage.

As is evident from FIGS. 37B and 37C, the expression of Homo sapiens maba1 (KIAA1324) transcripts detectable by the above amplicon in adenocarcinoma samples was significantly higher than in the non-cancerous samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above). Notably an over-expression of at least 5 fold was found in 36 out of 69 adenocarcinoma samples, specifically in 20 out of 43 serous carcinoma samples, in 8 out of 12 mucinous carcinoma samples and in 7 out of 10 endometroid samples. From FIG. 37B it can be observed that in serous samples over expression was mainly in patients with age below 50, in mucinus samples over expression was higher in patients with age below 50 and in endometroid over expression was mainly in patients with age above 50. From FIG. 37C it can be seen that in serous samples over expression was mainly in patients with stage I and II.

Statistical analysis was applied to verify the significance of these results, as described below.

The P value for the difference in the expression levels of Homo sapiens maba1 (KIAA1324) transcripts detectable by the above amplicon in Ovary adenocarcinoma samples versus the normal tissue samples was determined by T test as 8.76e-006. The P value for the difference in the expression levels of Homo sapiens maba1 (KIAA1324) transcripts detectable by the above amplicon in Ovary serous carcinoma samples versus the normal tissue samples was determined by T test as 1.55e-003. The P value for the difference in the expression levels of Homo sapiens maba1 (KIAA1324) transcripts detectable by the above amplicon in Ovary mucinous carcinoma samples versus the normal tissue samples was determined by T test as 4.29e-002. The P value for the difference in the expression levels of Homo sapiens maba1 (KIAA1324) transcripts detectable by the above amplicon in Ovary endometroid samples versus the normal tissue samples was determined by T test as 1.10e-002.

Threshold of 5 fold over expression was found to differentiate between adenocarcinoma and normal samples with P value of 1.43e-004 as checked by exact Fisher test Threshold of 5 fold over expression was found to differentiate between serous carcinoma and normal samples with P value of 1.78e-003 as checked by exact Fisher test. Threshold of 5 fold over expression was found to differentiate between mucinous carcinoma and normal samples with P value of 8.99e-004 as checked by exact Fisher test. Threshold of 5 fold over expression was found to differentiate between endometroid and normal samples with P value of 1.04e-003 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H13410_seg33F (SEQ ID NO: 238) forward primer; and H13410_seg33R (SEQ ID NO: 239) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg33 (SEQ ID NO: 240).

Expression of Homo sapiens maba1 H13410 Transcripts which are Detectable by Amplicon as Depicted in Sequence Name H13410_seg58 (SEQ ID NO: 243)) in Normal and Cancerous Ovary Tissues

Expression of Homo sapiens maba1 transcripts detectable by or according to seg58-H13410_seg58 (SEQ ID NO: 243) amplicon and primers H13410_seg58F (SEQ ID NO: 241) and H13410_seg58R (SEQ ID NO: 242) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and GAPDH (GenBank Accession No. BC026907 (SEQ ID NO:338); GAPDH amplicon (SEQ ID NO: 308)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 45, 46, 71 and 48, Table 1_{—}1 above), to obtain a value of fold up-regulation for each sample relative to the median of the normal PM samples.

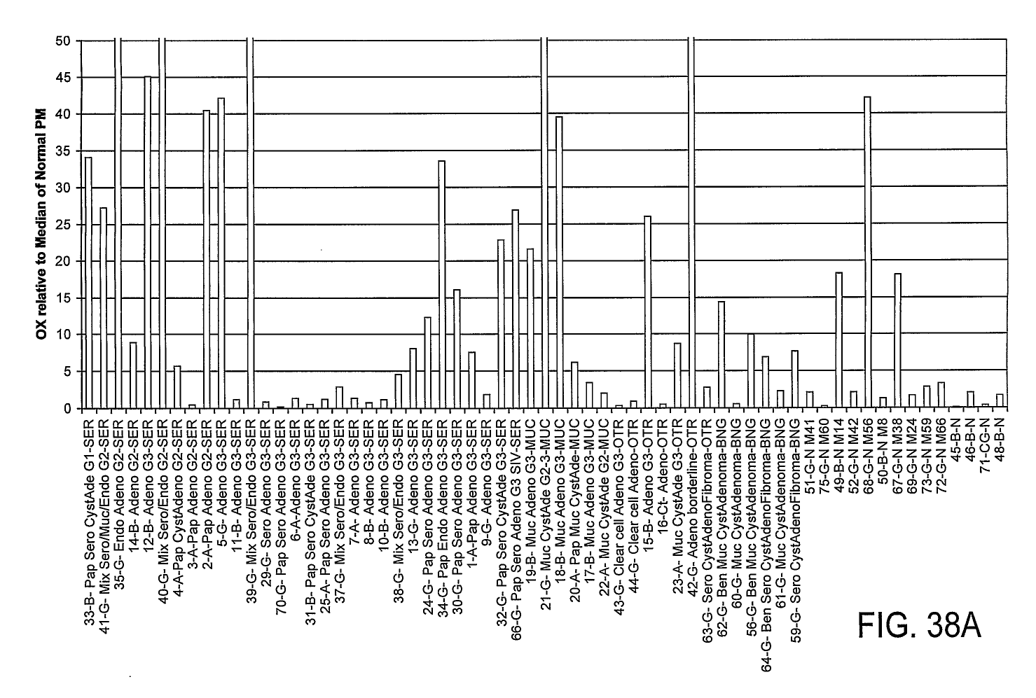

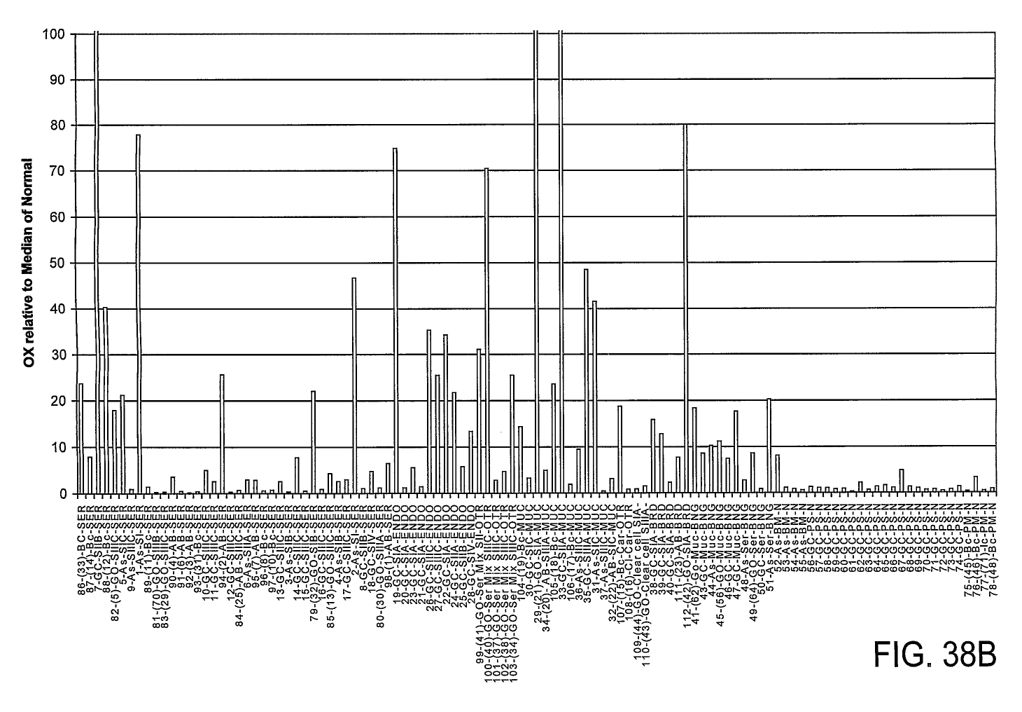

FIG. 38A is a histogram showing over expression of the Homo sapiens maba1 transcripts in cancerous Ovary samples relative to the normal samples.

As is evident from FIG. 38A, the expression of Homo sapiens maba1 transcripts detectable by the above amplicon in adenocarcinoma samples was significantly higher than in the non-cancerous samples (sample numbers 45, 46, 71 and 48, Table 1_{—}1 above). Notably an over-expression of at least 5 fold was found in 24 out of 43 adenocarcinoma samples, specifically in 17 out of 30 serous carcinoma samples and in 4 out of 6 mucinous carcinoma samples. Over expression was observed daily in patients with age lower than 50 or higher than 68.

Statistical analysis was applied to verify the significance of these results, as described below.

The P value for the difference in the expression levels of Homo sapiens maba1 transcripts detectable by the above amplicon in ovary serous carcinoma samples versus the normal tissue samples was determined by T test as 1.10e-003. The P value for the difference in the expression levels of Homo sapiens maba1 transcripts detectable by the above amplicon in all ovary adenocarcinoma samples versus the normal tissue samples was determined by T test as 6.01e-005.

Threshold of 5 fold over expression was found to differentiate between adenocarcinoma and normal samples with P value of 4.96e-002 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H13410_seg58F (SEQ ID NO: 241) forward primer; and H13410_seg58R (SEQ ID NO: 242) reverse primer).

The present invention also preferably encompasses any amplicon obtained through the use of any suitable primer pair; for example, for the above experiment, the following amplicon was obtained as a non-limiting illustrative example only of a suitable amplicon: H13410_seg58 (SEQ ID NO: 243).

Expression of Homo sapiens maba1 (KIAA1324) transcripts detectable by or according to seg58-H13410_seg58 (SEQ ID NO: 243) amplicon and primers H13410_seg58F (SEQ ID NO: 241) and H13410_seg58R (SEQ ID NO: 242) was further measured by real time PCR using enlarged panel as shown in Table 1_{—}5. In parallel the expression of three housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)) and G6PD (GenBank Accession No. NM_{—}000402 (SEQ ID NO:339); G6PD amplicon (SEQ ID NO: 311)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above), to obtain a value of fold up-regulation for each sample relative to the median of the normal samples.

FIGS. 38B and 38C are histograms showing over expression of the above-indicated Homo sapiens maba1 (KIAA1324) transcripts in cancerous Ovary samples relative to the normal samples. In FIG. 38B the samples are arranged according to the patient's age, and in FIG. 38C samples are arranged according to the patient's stage.

As is evident from FIGS. 38B and 38C, the expression of Homo sapiens maba1 (KIAA1324) transcripts detectable by the above amplicon in serous carcinoma, mucinous carcinoma and adenocarcinoma samples was significantly higher than in the non-cancerous samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above). Notably an over-expression of at least 5 fold was found in 32 out of 69 adenocarcinoma samples, specifically in 16 out of 43 serous carcinoma samples, in 7 out of 12 mucinous carcinoma samples and in 8 out of 10 endometroid samples. From FIG. 38B it can be observed that in serous samples over expression was mainly in patients with age below 50, in mucinus samples over expression was higher in patients with age below 50 and in endometroid over expression was mainly in patients with age above 50. From FIG. 38C it can be seen that in serous samples over expression was mainly in patients with stage I and II.

Statistical analysis was applied to verify the significance of these results, as described below.

The P value for the difference in the expression levels of Homo sapiens maba1 (KIAA1324) transcripts detectable by the above amplicon in Ovary adenocarcinoma samples versus the normal tissue samples was determined by T test as 3.54e-005. The P value for the difference in the expression levels of Homo sapiens maba1 (KIAA1324) transcripts detectable by the above amplicon in Ovary serous carcinoma samples versus the normal tissue samples was determined by T test as 7.18e-003. The P value for the difference in the expression levels of Homo sapiens maba1 (KIAA1324) transcripts detectable by the above amplicon in Ovary mucinous carcinoma samples versus the normal tissue samples was determined by T test as 2.55e-002. The P value for the difference in the expression levels of Homo sapiens maba1 (KIAA1324) transcripts detectable by the above amplicon in Ovary endometroid samples versus the normal tissue samples was determined by T test as 1.86e-002.

Threshold of 5 fold over expression was found to differentiate between adenocarcinoma and normal samples with P value of 2.30e-005 as checked by exact Fisher test. Threshold of 5 fold over expression was found to differentiate between serous carcinoma and normal samples with P value of 9.60e-004 as checked by exact Fisher test. Threshold of 5 fold over expression was found to differentiate between mucinous carcinoma and normal samples with P value of 3.54e-004 as checked by exact Fisher test. Threshold of 5 fold over expression was found to differentiate between endometroid and normal samples with P value of 9.81e-006 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H13410_seg58F (SEQ ID NO: 241) forward primer; and H13410_seg58R (SEQ ID NO: 242) reverse primer.

The present invention also preferably encompasses any amplicon obtained through the use of any suitable primer pair; for example, for the above experiment, the following amplicon was obtained as a non-limiting illustrative example only of a suitable amplicon: H13410_seg58 (SEQ ID NO: 243).

Expression of Homo sapiens maba1 (KIAA1324) H13410 Transcripts which are Detectable by Amplicon as Depicted in Sequence Name H13410_junc20-27F2R2 (SEQ ID NO: 385) in Normal and Cancerous Breast Tissues

Expression of Homo sapiens maba1 (KIAA1324) transcripts detectable by or according to junc20-27F2R2-H13410_junc20-27F2R2 (SEQ ID NO: 385) amplicon and primers H13410_junc20-27F2 (SEQ ID NO: 383) and H13410_junc20-27R2 (SEQ ID NO: 384) was measured by real time PCR. In parallel the expression of four housekeeping genes—G6PD (GenBank Accession No. NM_{—}000402 (SEQ ID NO:339); G6PD amplicon (SEQ ID NO: 311)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 57, 59, 60, 63, 66, 64, 56, 65, 67 and 58, Table 1_{—}3 above), to obtain a value of fold up-regulation for each sample relative to the median of the normal PM samples.

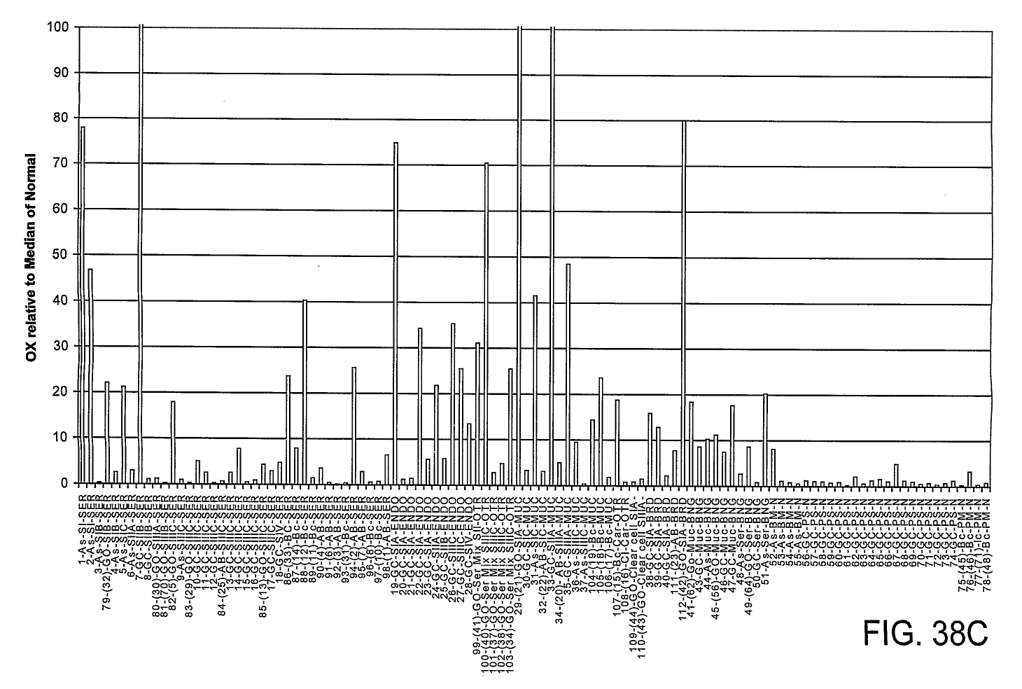

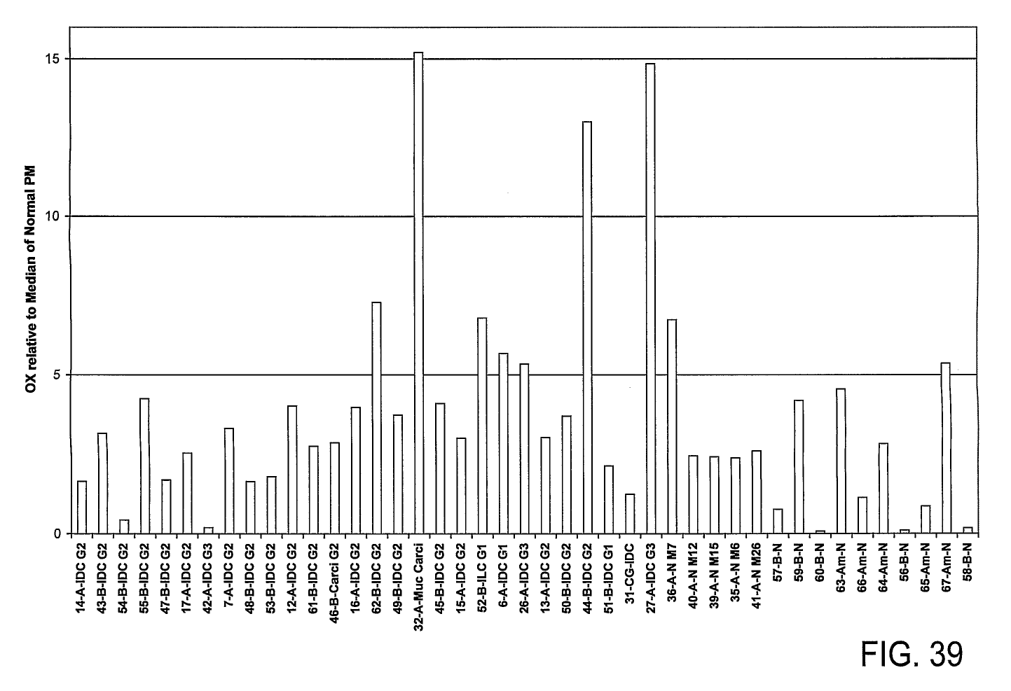

FIG. 39 is a histogram showing over expression of the above-indicated Homo sapiens maba1 (KIAA1324) transcripts in cancerous Breast samples relative to the normal samples.

As is evident from FIG. 39, the expression of Homo sapiens maba1 (KIAA1324) transcripts detectable by the above amplicon in cancer samples was higher than in the non-cancerous samples (sample numbers 57, 59, 60, 63, 66, 64, 56, 65, 67 and 58, Table 1_{—}3 above). Notably an over-expression of at least 5 fold was found in 7 out of 28 adenocarcinoma samples.

Statistical analysis was applied to verify the significance of these results, as described below.

The P value for the difference in the expression levels of Homo sapiens maba1 (KIAA1324) transcripts detectable by the above amplicon in Breast cancer samples versus the normal tissue samples was determined by T test as 2.05e-02.

The above values demonstrate statistical significance of the results.

Primer pairs are also optionally and preferably encompassed within the present invention; for example, for the above experiment, the following primer pair was used as a non-limiting illustrative example only of a suitable primer pair: H13410_junc20-27F2 (SEQ ID NO: 383) forward primer; and H13410_junc20-27R (SEQ ID NO: 384) reverse primer.

The present invention also preferably encompasses any amplicon obtained through the use of any suitable primer pair; for example, for the above experiment, the following amplicon was obtained as a non-limiting illustrative example only of a suitable amplicon: H13410_junc20-27F2R2 (SEQ ID NO: 385).

Expression of Homo sapiens maba1 (KIAA1324) H13410 Transcripts which are Detectable by Amplicon as Depicted in Sequence Name H13410_junc20-27F2R2 (SEQ ID NO: 385) in Different Normal Tissues

Expression of Homo sapiens maba1 (KIAA1324) transcripts detectable by or according to junc20-27F2R2-H13410_junc20-27F2R2 (SEQ ID NO: 385) amplicon and primers H13410_junc20-27F2 (SEQ ID NO: 383) and H13410_junc20-27R2 (SEQ ID NO: 384) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the geometric mean of the quantities of the housekeeping genes. The normalized quantity of each RT sample was then divided by the median of the quantities of the ovary samples (sample numbers 19 and 20, Table 1_{—}9 above), to obtain a value of relative expression of each sample relative to the median of the ovary samples.

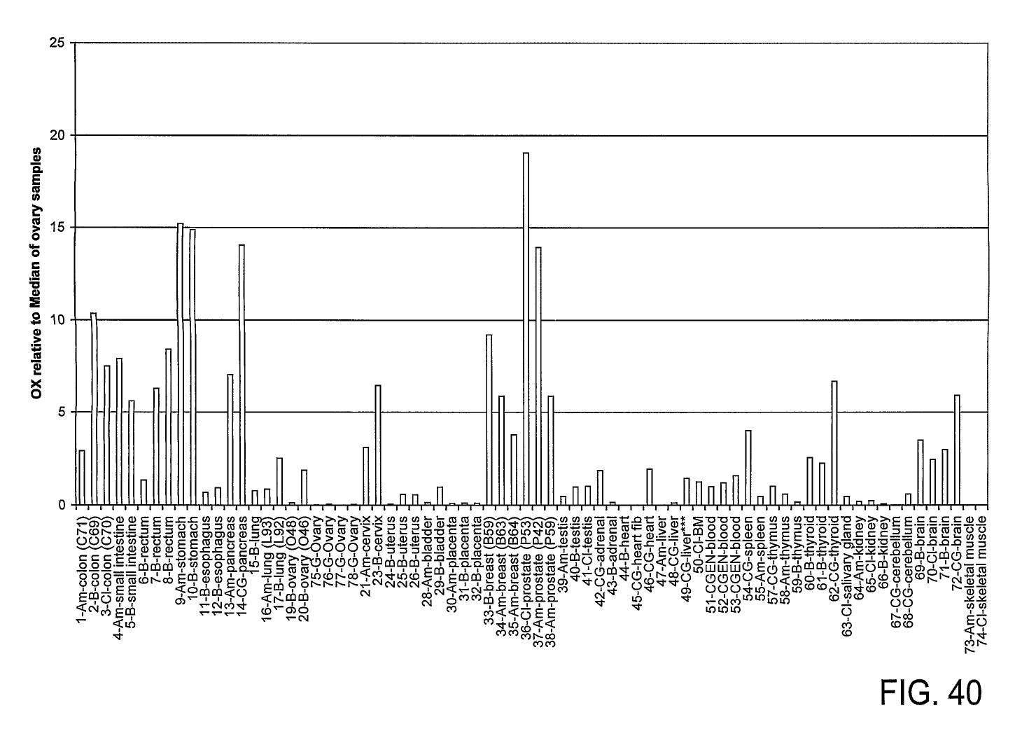

FIG. 40 is a histogram showing over expression of the above-indicated Homo sapiens maba1 (KIAA1324) H13410 transcripts which are detectable by amplicon as depicted in sequence name H13410_junc20-27F2R2 (SEQ ID NO: 385) in different normal samples.

Expression of Homo sapiens maba1 (KIAA1324) H13410 Transcripts which are Detectable by Amplicon as Depicted in Sequence Name H13410_junc20-27F2R2 (SEQ ID NO: 385) in Normal and Cancerous Ovary Tissues

Expression of Homo sapiens maba1 (KIAA1324) transcripts detectable by or according to junc20-27F2R2-H13410_junc20-27F2R2 (SEQ ID NO: 385) amplicon and primers H13410_junc20-27F2 (SEQ ID NO: 383) and H13410_junc20-27R2 (SEQ ID NO: 384) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and GAPDH (GenBank Accession No. BC026907 (SEQ ID NO:338); GAPDH amplicon (SEQ ID NO: 308)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 45, 46, 71 and 48, Table 1_{—}1 above), to obtain a value of fold up-regulation for each sample relative to the median of the normal PM samples.

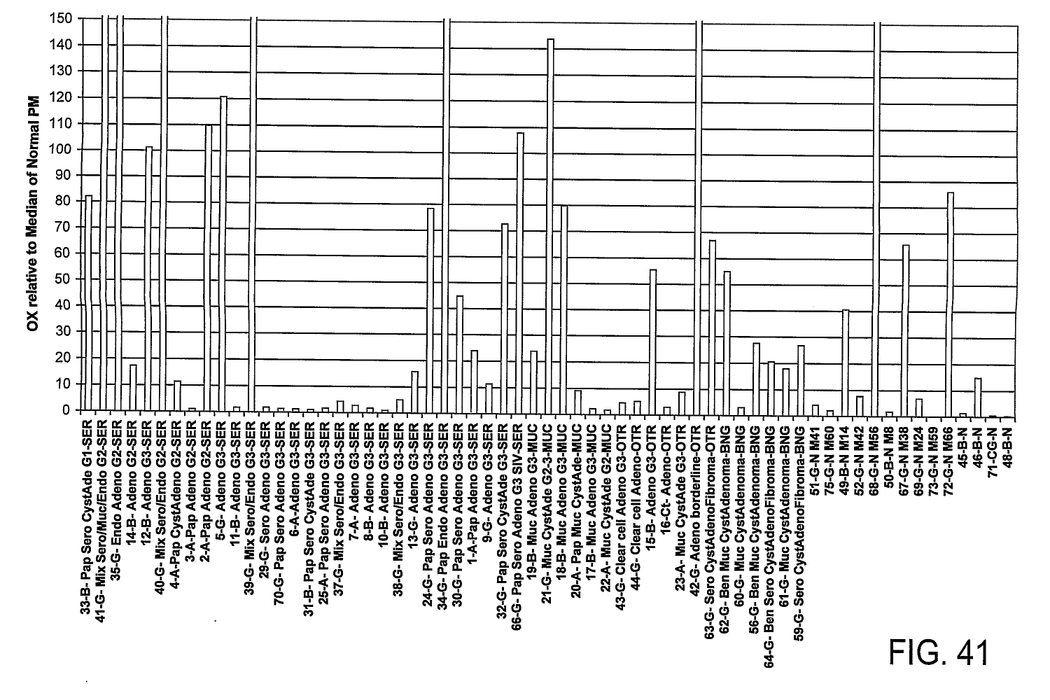

FIG. 41 is a histogram showing over expression of the above-indicated Homo sapiens maba1 (KIAA1324) transcripts in cancerous Ovary samples relative to the normal samples.

As is evident from FIG. 41, the expression of Homo sapiens maba1 (KIAA1324) transcripts detectable by the above amplicon in adenocarcinoma samples was higher than in the non-cancerous samples (sample numbers 45, 46, 71 and 48, Table 1_{—}1 above), mainly in patients below 50 or above 68 years old. Notably an over-expression of at least 5 fold was found in 28 out of 43 adenocarcinoma samples, specifically in 19 out of 30 serous carcinoma samples and in 4 out of 6 mucinous carcinoma samples.

Statistical analysis was applied to verify the significance of these results, as described below.

The P value for the difference in the expression levels of Homo sapiens maba1 (KIAA1324) transcripts detectable by the above amplicon in Ovary serous carcinoma samples versus the normal tissue samples was determined by T test as 1.55e-03. The P value for the difference in the expression levels of Homo sapiens maba1 (KIAA1324) transcripts detectable by the above amplicon in Ovary adenocarcinoma samples versus the normal tissue samples was determined by T test as 2.15e-04.

The above values demonstrate statistical significance of the results.

Primer pairs are also optionally and preferably encompassed within the present invention; for example, for the above experiment, the following primer pair was used as a non-limiting illustrative example only of a suitable primer pair: H13410_junc20-27F2 (SEQ ID NO: 383) forward primer; and H13410_junc20-27R2 (SEQ ID NO: 384) reverse primer.

The present invention also preferably encompasses any amplicon obtained through the use of any suitable primer pair; for example, for the above experiment, the following amplicon was obtained as a non-limiting illustrative example only of a suitable amplicon: H13410_junc20-27F2R2 (SEQ ID NO: 385).

### Description for Cluster H61775

A description of some of these segments according to the present invention is now provided. Cluster H61775 (internal ID 8683039) features 2 transcript(s) and 3 segment(s) of interest, the names for which are given in Tables 82 and 83, respectively, the sequences themselves are given at the Sequence Listing. The selected protein variants are given in table 84.

TABLE 82
Transcripts of interest
Transcript Name

H61775_T22 (SEQ ID NO: 345)
H61775_T23 (SEQ ID NO: 346)

TABLE 83
Segments of interest
Segment Name

H61775_N6 (SEQ ID NO: 347)
H61775_N8 (SEQ ID NO: 348)
H61775_N5 (SEQ ID NO: 349)

TABLE 84
Proteins of interest

| Protein Name | Corresponding Transcript(s) |
|---|---|
| H61775_P17 (SEQ ID NO: 354) | H61775_T22 (SEQ ID NO: 345) |
| H61775_P18 (SEQ ID NO: 355) | H61775_T23 (SEQ ID NO: 346) |

These sequences are variants of the known protein immunoglobulin superfamily (SEQ ID NO:350), member 9 (SwissProt accession identifier NP_{—}065840), referred to herein as the previously known protein.

PCT/IB2005/004037 and US Patent Application No. US 2006-0046257, filed on Jan. 27, 2005 and assigned to the applicant of the present invention, disclose the sequences of H61775_T22 (SEQ ID NO:345), and the encoded H61775_P17 (SEQ ID NO:354) of H61775 contig, and assays and methods of use thereof in the diagnosis of lung cancer. WO2005IB0002555 and US Patent Application No. US 2006-0040278, filed on Jan. 27, 2005 and assigned to the applicant of the present invention, disclose the sequences of H61775_T22 (SEQ ID NO:345) and H61775_P17 (SEQ ID NO:354) of H61775 contig, and assays and methods of use thereof in the diagnosis of ovarian cancer.

Unexpectedly, the sequences of H61775_T22 (SEQ ID NO:345) and H61775_P17 (SEQ ID NO:354) of H61775 contig are now disclosed to be differentially expressed in breast cancer, and therefore may be contemplated within the scope of the present invention as diagnostic markers for this disease.

Unexpectedly, the previously disclosed sequence of H61775_T23 (SEQ ID NO:346) has now been found to have a variant form, disclosed herein as SEQ ID NO:346. This variant form features a single nucleotide deletion after a base 508. As a result of this deletion, a completely new protein, H61775_P18 (SEQ ID NO:355), has now been found. This protein has a significantly different structure from other members of this protein family. The novel H61775 variant, H61775_T23 (SEQ ID NO:346) and the encoded H61775_P18 (SEQ ID NO:355), are now disclosed as an efficient diagnostic markers for breast cancer, ovarian cancer and lung cancer.

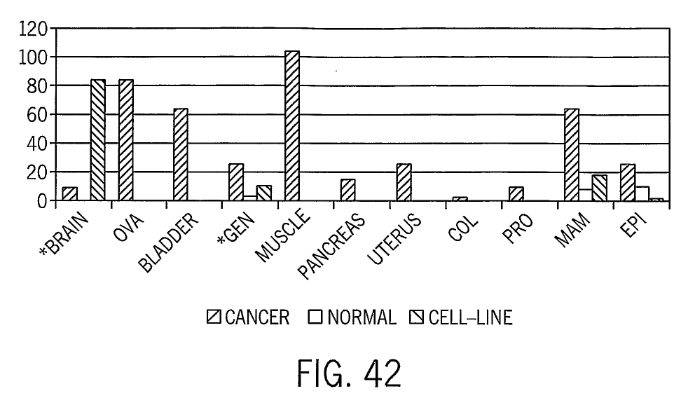

Cluster H61775 can be used as a diagnostic marker according to overexpression of transcripts of this cluster in cancer. Expression of such transcripts in normal tissues is also given according to the previously described methods. The term “number” in the left hand column of the table and the numbers on the y-axis of the FIG. 42 refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 42 and Table 85. This cluster is overexpressed (at least at a minimum level) in the following pathological conditions: brain malignant tumors and a mixture of malignant tumors from different tissues.

TABLE 85
Normal tissue distribution

| Name of Tissue | Number |
|---|---|
| brain | 0 |
| ovary | 0 |
| bladder | 0 |
| general | 3 |
| muscle | 0 |
| pancreas | 0 |
| uterus | 0 |
| colon | 0 |
| prostate | 0 |
| breast | 8 |
| epithelial | 10 |

TABLE 86
P values and ratios for expression in cancerous tissue

| Name of Tissue | P1 | P2 | SP1 | R3 | SP2 | R4 |
|---|---|---|---|---|---|---|
| brain | 4.4e−02 | 1.2e−02 | N/A | N/A | 4.8e−05 | 9.2 |
| ovary | 3.8e−01 | 4.2e−01 | 1.5e−01 | 2.4 | 2.7e−01 | 1.9 |
| bladder | 3.1e−01 | 3.8e−01 | 3.2e−01 | 2.5 | 4.6e−01 | 1.9 |
| general | 1.6e−06 | 8.0e−05 | 2.4e−07 | 5.9 | 4.1e−06 | 3.9 |
| muscle | 2.3e−01 | 2.9e−01 | 1.5e−01 | 6.6 | 4.0e−01 | 2.5 |
| pancreas | 3.1e−01 | 4.2e−01 | 4.2e−01 | 2.4 | 5.3e−01 | 1.9 |
| uterus | 8.7e−02 | 2.3e−01 | 4.4e−01 | 2.1 | 6.4e−01 | 1.5 |
| colon | 4.7e−01 | 5.5e−01 | N/A | N/A | N/A | N/A |
| prostate | 7.1e−01 | 7.6e−01 | 6.7e−01 | 1.5 | 7.5e−01 | 1.3 |
| breast | 4.7e−01 | 4.2e−01 | 3.2e−01 | 2.1 | 4.6e−01 | 1.6 |
| epithelial | 2.6e−02 | 1.3e−01 | 3.3e−02 | 2.0 | 3.7e−01 | 1.1 |

AS noted above, cluster H61775 features 2 transcript(s), which were listed in Table 82 above. These transcript(s) encode for protein(s) which are variant(s) of protein immunoglobulin superfamily (SEQ ID NO:350), member 9. A description of each variant protein according to the present invention is now provided.

Variant protein H61775_P17 (SEQ ID NO:354) according to the present invention has an amino acid sequence encoded by transcript(s) H61775_T22 (SEQ ID NO:345). An alignment is given to the known protein (immunoglobulin superfamily member 9, SEQ ID NOs:350-353) in the alignment table on the attached CDROM. A brief description of the relationship of the variant protein according to the present invention to each such aligned protein is as follows:

### 1. Comparison Report Between H61775_P17 (SEQ ID NO:354) and Known Protein(s) NP_{—}065840 (SEQ ID NO: 351):

A. An isolated chimeric polypeptide encoding for H61775_P17 (SEQ ID NO:354), comprising a first amino acid sequence being at least about 90% homologous to MVWCLGLAVLSLVISQGADGRGKPEVVSVVGRA corresponding to amino acids 1-33 of known protein(s) NP_{—}065840 (SEQ ID NO: 351), which also corresponds to amino acids 1-33 of H61775_P17 (SEQ ID NO:354), a bridging amino acid G corresponding to amino acid 34 of H61775_P17 (SEQ ID NO:354), a second amino acid sequence being at least about 90% homologous to ESVVLGCDLLPPAGRPPLHVIEWLRFGFLLPIFIQFGLYSPRIDPDYVG corresponding to amino acids 35-83 of known protein(s) NP_{—}065840 (SEQ ID NO: 351), which also corresponds to amino acids 35-83 of H61775_P17 (SEQ ID NO:354), and a third amino acid sequence being at least about 70%, optionally at least 80%, preferably at least 85%, more preferably at least 90% and most preferably at least 95% homologous to a polypeptide having the sequence DCGFPAFRELKRAETVSPVFFTRRCIWEDLKSTGFSPAGGGRPPGGGPRTQEDSGLPCWRSSCSVTLQV (SEQ ID NO: 532) corresponding to amino acids 84-152 of H61775_P17 (SEQ ID NO:354), wherein said first amino acid sequence, bridging amino acid, second amino acid sequence and third amino acid sequence are contiguous and in a sequential order.

B. An isolated polypeptide encoding for an edge portion of H61775_P17 (SEQ ID NO:354), comprising an amino acid sequence being at least about 70%, optionally at least about 80%, preferably at least about 85%, more preferably at least about 90% and most preferably at least about 95% homologous to the sequence

(SEQ ID NO: 532)

DCGFPAFRELKRAETVSPVFFTRRCIWEDLKSTGFSPAGGGRPPGGGPRT
QEDSGLPCWRSSCSVTLQV
of

(SEQ ID NO: 354)

H61775_P17.

### 2. Comparison Report Between H61775_P17 (SEQ ID NO:354) and Known Protein(S) Q9P2J2_Human (SEQ ID NO: 352):

A. An isolated chimeric polypeptide encoding for H61775_P17 (SEQ ID NO:354), comprising a first amino acid sequence being at least about 90% homologous to MVWCLGLAVLSLVISQGADGRGKPEVVSVVGRAGESVVLGCDLLPPAGRPPLHVIEWLRFGFLLPIFIQFG LYSPRIDPDYVG corresponding to amino acids 11-93 of known protein(s) Q9P2J2_HUMAN (SEQ ID NO: 352), which also corresponds to amino acids 1-83 of H61775_P17 (SEQ ID NO:354), and a second amino acid sequence being at least about 70%, optionally at least 80%, preferably at least 85%, more preferably at least 90% and most preferably at least 95% homologous to a polypeptide having the sequence DCGFPAFRELKRAETVSPVFFTRRCIWEDLKSTGFSPAGGGRPPGGGPRTQEDSGLPCWRSSCSVTLQV (SEQ ID NO: 532) corresponding to amino acids 84-152 of H61775_P17 (SEQ ID NO:354), wherein said, first amino acid sequence and second amino acid sequence are contiguous and in a sequential order.

B. An isolated polypeptide encoding for an edge portion of H61775_P17 (SEQ ID NO:354), comprising an amino acid sequence being at least about 70%, optionally at least about 80%, preferably at least about 85%, more preferably at least about 90% and most preferably at least about 95% homologous to the sequence

(SEQ ID NO: 532)

DCGFPAFRELKRAETVSPVFFTRRCIWEDLKSTGFSPAGGGRPPGGGPRT
QEDSGLPCWRSSCSVTLQV
of

(SEQ ID NO: 354)

H61775_P17.

The localization of the variant protein was determined according to results from a number of different software programs and analyses, including analyses from SignalP and other specialized programs. The variant protein is believed to be secreted.

Variant protein H61775_P17 (SEQ ID NO:354) also has the following non-silent SNPs (Single Nucleotide Polymorphisms) as listed in Table 87, (given according to their position(s) on the amino acid sequence, with the alternative amino acid(s) listed).

TABLE 87
Amino acid mutations

| SNP position(s) on amino acid | |
|---|---|
| sequence | Alternative amino acid(s) |
| 14 | I -> T |
| 34 | G -> E |
| 48 | G -> R |
| 91 | R -> * |
| 138 | G -> R |

Variant protein H61775_P17 (SEQ ID NO:354) is encoded by the following transcript(s): H61775_T22 (SEQ ID NO:345), for which the coding portion starts at position 261 and ends at position 716. The transcript also has the following SNPs as listed in Table 88 (given according to their position on the nucleotide sequence, with the alternative nucleic acid listed).

TABLE 88
Nucleic acid SNPs

| Polymorphism | SNP position(s) on nucleotide sequence |
|---|---|
| T -> C | 117, 200, 222, 301, 566 |
| G -> A | 361, 377 |
| G -> C | 402, 672 |
| C -> T | 531 |

Variant protein H61775_P18 (SEQ ID NO:355) according to the present invention has an amino acid sequence encoded by transcript(s) H61775_T23 (SEQ ID NO:346). An alignment is given to the known protein (immunoglobulin superfamily member 9, SEQ ID NOs:350-353) in the alignment table on the attached CDROM. A brief description of the relationship of the variant protein according to the present invention to each such aligned protein is as follows:

1. Comparison Report Between H61775_P18 (SEQ ID NO:355) and known protein(s) NP_{—}065840 (SEQ ID NO: 351):

A. An isolated chimeric polypeptide encoding for H61775_P18 (SEQ ID NO:355), comprising a first amino acid sequence being at least about 90% homologous to MVWCLGLAVLSLVISQGADGRGKPEVVSVVGRA corresponding to amino acids 1-33 of known protein(s) NP_{—}65840 (SEQ ID NO: 351), which also corresponds to amino acids 1-33 of H61775_P18 (SEQ ID NO:355), a bridging amino acid G corresponding to amino acid 34 of H61775_P18 (SEQ ID NO:355), a second amino acid sequence being at least about 90% homologous to ESVVLGCDLLPPAGRPPLHVIEWLRFGFLLPIFIQFGLYSPRIDPDYVG corresponding to amino acids 35-83 of known protein(s) NP_{—}65840 (SEQ ID NO: 351), which also corresponds to amino acids 35-83 of H61775_P18 (SEQ ID NO:355), and a third amino acid sequence being at least about 70%, optionally at least 80%, preferably at least 85%, more preferably at least 90% and most preferably at least 95% homologous to a polypeptide having the sequence KSSPQVGGREVSARKVGWVESHKASYERLCPCSDLISSSAALTLLNKHGCAPKQC (SEQ ID NO: 505) corresponding to amino acids 84-138 of H61775_P18 (SEQ ID NO:355), wherein said first amino acid sequence, bridging amino acid, second amino acid sequence and third amino acid sequence are contiguous and in a sequential order.

B. An isolated polypeptide encoding for an edge portion of H61775_P18 (SEQ ID NO:355), comprising an amino acid sequence being at least about 70%, optionally at least about 80%, preferably at least about 85%, more preferably at least about 90% and most preferably at least about 95% homologous to the sequence

(SEQ ID NO: 505)

KSSPQVGGREVSARKVGWVESHKASYERLCPCSDLISSSAALTLLNKHGC
APKQC
of

(SEQ ID NO: 355)

H61775_P18.

### 2. Comparison Report Between H61775_P18 (SEQ ID NO:355) and Known Protein(s) Q9P2J2_HUMAN (SEQ ID NO: 352):

A. An isolated chimeric polypeptide encoding for H61775_P18 (SEQ ID NO:355), comprising a first amino acid sequence being at least about 90% homologous to MVWCLGLAVLSLVISQGADGRGKPEVVSVVGRAGESVVLGCDLLPPAGRPPLHVIEWLRFGFLLPIFIQFG LYSPRIDPDYVG corresponding to amino acids 11-93 of known protein(s) Q9P2J2_HUMAN (SEQ ID NO: 352), which also corresponds to amino acids 1-83 of H61775_P18 (SEQ ID NO:355), and a second amino acid sequence being at least about 70%, optionally at least 80%, preferably at least 85%, more preferably at least 90% and most preferably at least 95% homologous to a polypeptide having the sequence KSSPQVGGREVSARKVGWVESHKASYERLCPCSDLISSSAALTLLNKHGCAPKQC (SEQ ID NO: 505) corresponding to amino acids 84-138 of H61775_P18 (SEQ ID NO:355), wherein said, first amino acid sequence and second amino acid sequence are contiguous and in a sequential order.

The localization of the variant protein was determined according to results from a number of different software programs and analyses, including analyses from SignalP and other specialized programs. The variant protein is believed to be secreted.

Variant protein H61775_P18 (SEQ ID NO:355) also has the following non-silent SNPs (Single Nucleotide Polymorphisms) as listed in Table 89, (given according to their position(s) on the amino acid sequence, with the alternative amino acid(s) listed).

TABLE 89
Amino acid mutations

| SNP position(s) on amino acid | |
|---|---|
| sequence | Alternative amino acid(s) |
| 14 | I -> T |
| 34 | G -> E |
| 48 | G -> R |
| 112 | L -> H |

Variant protein H61775_P18 (SEQ ID NO:355) is encoded by the following transcript(s): H61775_T23 (SEQ ID NO:346), for which coding portion starts at position 261 and ends at position 674. The transcript also has the following SNPs as listed in Table 90 (given according to their position on the nucleotide sequence, with the alternative nucleic acid listed; H61775_P18 (SEQ ID NO:355).

TABLE 90
Nucleic acid SNPs

| Polymorphism | SNP position(s) on nucleotide sequence |
|---|---|
| T -> C | 117, 200, 222, 301 |
| G -> A | 361, 377 |
| G -> C | 402 |
| T -> A | 595 |

As noted above, cluster H61775 features 3 segment(s), which were listed in Table 82 above and for which the sequence(s) are given in the Sequence Listing. These segment(s) are portions of nucleic acid sequence(s) which are described herein separately because they are of particular interest. A description of some of these segments according to the present invention is now provided.

Segment cluster H61775_N6 (SEQ ID NO:347) according to the present invention is supported by 1 libraries. The number of libraries was determined as previously described. This segment can be found in the following transcript(s): H61775_T23 (SEQ ID NO:346). Table 91 below describes the starting and ending position of this segment on each transcript.

TABLE 91
Segment location on transcripts

| | Segment | Segment |
|---|---|---|
| Transcript name | starting position | ending position |
| H61775_T23 (SEQ ID NO: 346) | 514 | 713 |

Segment Cluster H61775_N8 (SEQ ID NO:348) according to the present invention is supported by 5 libraries. The number of libraries was determined as previously described. This segment can be found in the following transcript(s): H61775_T22 (SEQ ID NO:345). Table 92 below describes the starting and ending position of this segment on each transcript.

TABLE 92
Segment location on transcripts

| | Segment | Segment |
|---|---|---|
| Transcript name | starting position | ending position |
| H61775_T22 (SEQ ID NO: 345) | 508 | 1202 |

According to an optional embodiment of the present invention, short segments related to the above cluster are also provided. These segments are up to about 120 bp in length, and so are included in a separate description.

Segment cluster H61775_N5 (SEQ ID NO:349) according to the present invention is supported by 3 libraries. The number of libraries was determined as previously described. This segment can be found in the following transcript(s): H61775 T23 (SEQ ID NO:346). Table 93 below describes the starting and ending position of this segment on each transcript.

TABLE 93
Segment location on transcripts

| | Segment | Segment |
|---|---|---|
| Transcript name | starting position | ending position |
| H61775_T23 (SEQ ID NO: 346) | 508 | 513 |

Expression of Homo sapiens immunoglobulin superfamily, member 9 (IGSF9) transcripts detectable by or according to segments as described below was measured by real time PCR: seg8—H61775 seg8 (SEQ ID NO: 267) amplicon and primers H61775 seg8F2 (SEQ ID NO: 265) and H61775 seg8R2 (SEQ ID NO: 266); seg12WT-H61775_seg12WT (SEQ ID NO: 270) amplicon and primers H61775_seg12WTF (SEQ ID NO: 268) and H61775_seg12WTR (SEQ ID NO: 269); seg5-6F1R1—H61775_seg5-6F1R1 (SEQ ID NO: 394) amplicon and primers H61775_seg5-6F (SEQ ID NO: 392) and H61775_seg5-6R (SEQ ID NO: 393). The sequences of the corresponding amplicons and primers are given below:

Forward primer H61775 seg8F2 (SEQ ID NO: 265):
GAAGGCTCTTGTCACTTAGTAGCCAT
Reverse primer H61775 seg8R2 (SEQ ID NO: 266):
TGTCACCATATTTAATCCTCCCAA
Amplicon H61775 seg8 (SEQ ID NO: 267):
GAAGGCTCTTGTCACTTACTAGCCATGTGATTTTGGAAAGAAACTTAACA
TTAATTCCTTCAGCTACAATGGAATTCTTGGGAGGATTAAATATGGTGAC
A
Forward Primer (H61775_seg12WTF (SEQ ID NO: 268)):
CCTGCTGTGTTGGAAGTGCA
Reverse Primer (H61775_seg12WTR (SEQ ID NO: 269)):
AAGGTCCTTTCCTCGGAGCT
Amplicon (H61775_seg12WT (SEQ ID NO: 270)):
CCTGCTGTGTTGGAAGTGCAGGAACTGGAGCCTGTGACCCTGCGTTGTGT
GGCCCGTGGCAGCCCCCTGCCTCATGTGACGTGGAAGCTCCGAGGAAAGG
ACCTT
Forward Primer (H61775_seg5-6F) (SEQ ID NO: 392):
GTAAGAGTTCTCCTCAGGTGGGAG
Reverse Primer (H61775_seg5-6R) (SEQ ID NO: 393):
TTAAGGCAGCAGAGCTGGAGAT
Amplicon (H61775_seg5-6F1R1) (SEQ ID NO:394):
GTAAGAGTTCTCCTCAGGTGGGAGGTAGGGAGGTATCAGCAAGAAAGGTG
GGCTGGGTAGAGTCGCACAAGGCCTCCTATGAACGGCTTTGTCCCTGCTC
TGATCTCATCTCCAGCTCTGCTGCCTTAA

Expression of Homo sapiens Immunoglobulin Superfamily, Member 9 (IGSF9) H61775 Transcripts which are Detectable by Amplicon as Depicted in Sequence Name H61775 seg8 (SEQ ID NO: 267) in Normal and Cancerous Breast tissues

Expression of Homo sapiens immunoglobulin superfamily, member 9 (IGSF9) transcripts detectable by or according to seg8—H61775 seg8 (SEQ ID NO: 267) amplicon and primers H61775 seg8F2 (SEQ ID NO: 265) and H61775 seg8R2 (SEQ ID NO: 266) was measured by real time PCR. In parallel the expression of four housekeeping genes—PBGD (GenBank Accession No. BC019323 (SEQ ID NO:336); amplicon—PBGD-amplicon (SEQ ID NO: 302)), HPRT1 (GenBank Accession No. NM_{—}000194 (SEQ ID NO:337); amplicon—HPRT1-amplicon (SEQ ID NO: 305) (SEQ ID NO: 305)), SDHA (GenBank Accession No. NM_{—}004168 (SEQ ID NO:335); amplicon—SDHA-amplicon (SEQ ID NO:299)), G6PD (GenBank Accession No. NM_{—}000402 (SEQ ID NO:339); G6PD amplicon (SEQ ID NO: 311)), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 56-60, 63-67, Table 1_{—}3 above), to obtain a value of fold up-regulation for each sample relative to the median of the normal PM samples.

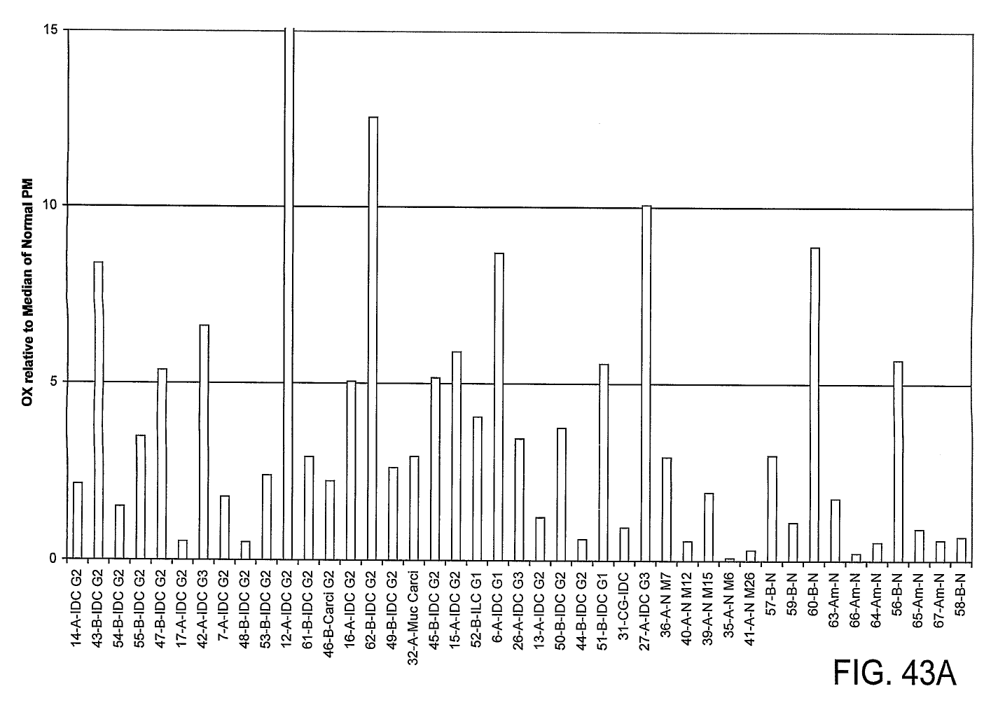

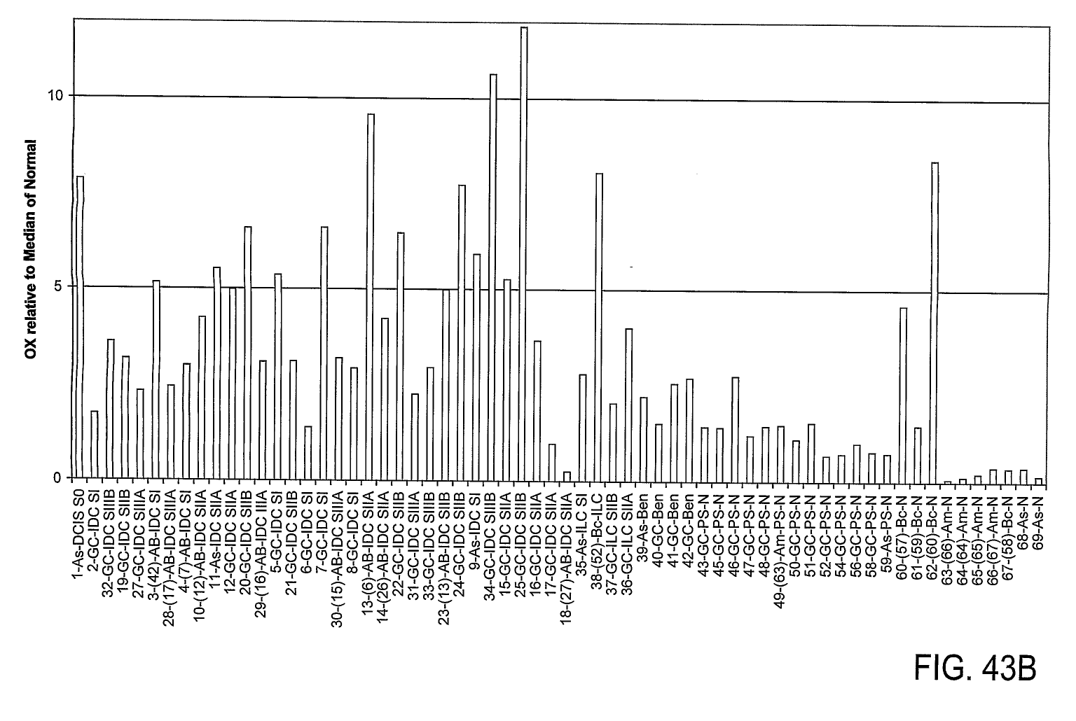

FIG. 43A is a histogram showing over expression of the Homo sapiens immunoglobulin superfamily, member 9 (IGSF9) transcripts detectable by or according to H61775 seg8 (SEQ ID NO: 267) in cancerous Breast samples relative to the normal samples.

As is evident from FIG. 43A, the expression of Homo sapiens immunoglobulin superfamily, member 9 (IGSF9) transcripts detectable by the above amplicon in cancer samples was higher than in the non-cancerous samples (Sample Nos. 56-60, 63-67 Table 1_{—}3). Notably an over-expression of at least 5 fold was found in 11 out of 28 adenocarcinoma samples.

Primer pairs are also optionally and preferably encompassed within the present invention; for example, for the above experiment, the following primer pair was used as a non-limiting illustrative example only of a suitable primer pair: H61775 seg8F2 (SEQ ID NO: 265) forward primer; and H61775 seg8R2 (SEQ ID NO: 266) reverse primer.

The present invention also preferably encompasses any amplicon obtained through the use of any suitable primer pair; for example, for the above experiment, the following amplicon was obtained as a non-limiting illustrative example only of a suitable amplicon: H61775 seg8 (SEQ ID NO: 267).

Expression of Homo sapiens immunoglobulin superfamily, member 9 (IGSF9) transcripts detectable by or according to seg8F2R—H61775_seg8F2R2 (SEQ ID NO: 267) amplicon and primers H61775_seg8F2 (SEQ ID NO: 265) and H61775_seg8R2 (SEQ ID NO: 266) was further measured by real time PCR using enlarged panel as shown in Table 1_{—}7. In parallel the expression of four housekeeping genes—G6PD (GenBank Accession No. NM_{—}000402 (SEQ ID NO:339); G6PD amplicon (SEQ ID NO: 311)), RPL19 (GenBank Accession No. NM_{—}000981 (SEQ ID NO:342); RPL19 amplicon (SEQ ID NO: 320)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 43, 45, 46, 47, 48, 49, 50, 51, 52, 54, 56, 58, 59, 60, 61, 62, 63, 64, 65, 66, 67, 68 and 69, Table 1_{—}7 above), to obtain a value of fold up-regulation for each sample relative to the median of the normal samples.

FIG. 43B is a histogram showing low over expression of the above-indicated Homo sapiens immunoglobulin superfamily, member 9 (IGSF9) transcripts in cancerous Breast samples relative to the normal samples.

As is evident from FIG. 43B, the expression of Homo sapiens immunoglobulin superfamily, member 9 (IGSF9) transcripts detectable by the above amplicon in cancer samples was higher than in the non-cancerous samples (sample numbers 39, 41, 42, 43, 45, 46, 47, 48, 49, 50, 51, 52, 54, 56, 58, 59, 60, 61, 62, 63, 64, 65, 66, 67, 68 and 69, Table 1_{—}7 above). Notably an over-expression of at least 5 fold was found in 14 out of 37 adenocarcinoma samples.

Statistical analysis was applied to verify the significance of these results, as described below.

The P value for the difference in the expression levels of Homo sapiens immunoglobulin superfamily, member 9 (IGSF9) transcripts detectable by the above amplicon in Breast cancer samples versus the normal tissue samples was determined by T test as 2.66e-06.

Threshold of 5 fold over expression was found to differentiate between cancer and normal samples with P value of 2.82e-03 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H61775_seg8F2 (SEQ ID NO: 265) forward primer; and H61775_seg8R2 (SEQ ID NO: 266) reverse primer.

The present invention also preferably encompasses any amplicon obtained through the use of any suitable primer pair; for example, for the above experiment, the following amplicon was obtained as a non-limiting illustrative example only of a suitable amplicon: H61775_seg8F2R2 (SEQ ID NO: 267).

Expression of Homo sapiens Immunoglobulin Superfamily, Member 9 (IGSF9) H61775 Transcripts which are Detectable by Amplicon as Depicted in Sequence name H61775_seg12WT (SEQ ID NO: 270) in Normal and Cancerous Ovary Tissues

Expression of Homo sapiens immunoglobulin superfamily, member 9 (IGSF9) transcripts detectable by or according to seg12WT—H61775 seg12WT (SEQ ID NO: 270) amplicon and primers H61775_seg12WTF (SEQ ID NO: 268) and H61775_seg12WTR (SEQ ID NO: 269) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and GAPDH (GenBank Accession No. BC026907 (SEQ ID NO:338); GAPDH amplicon (SEQ ID NO: 308)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 45, 46, 71 and 48, Table 1_{—}1 above), to obtain a value of fold up-regulation for each sample relative to the median of the normal PM samples.

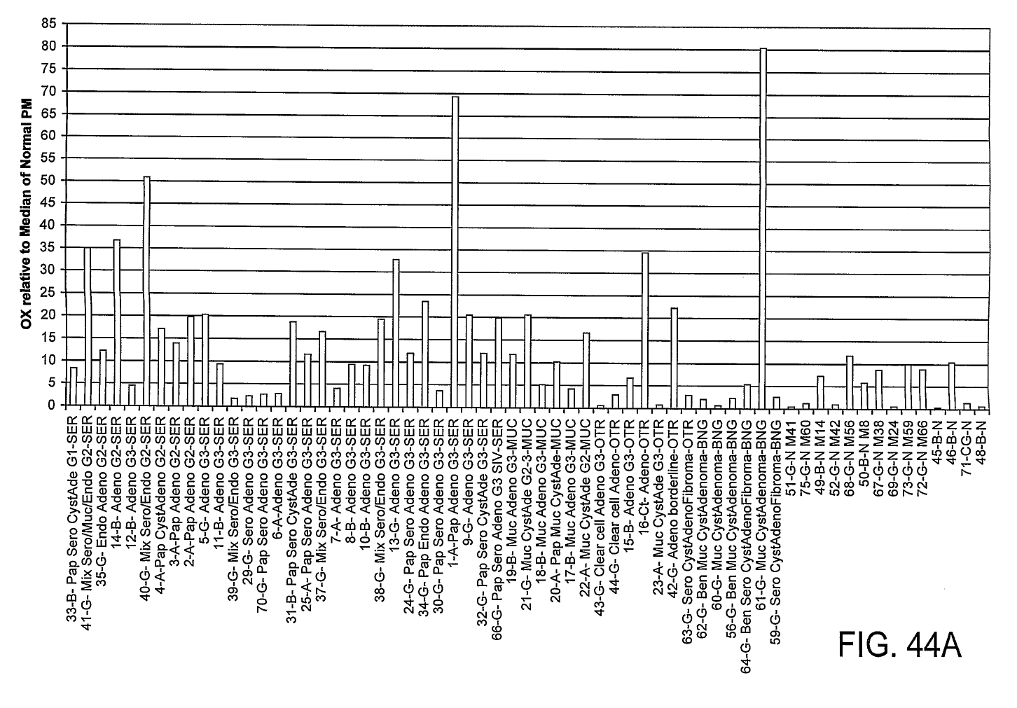

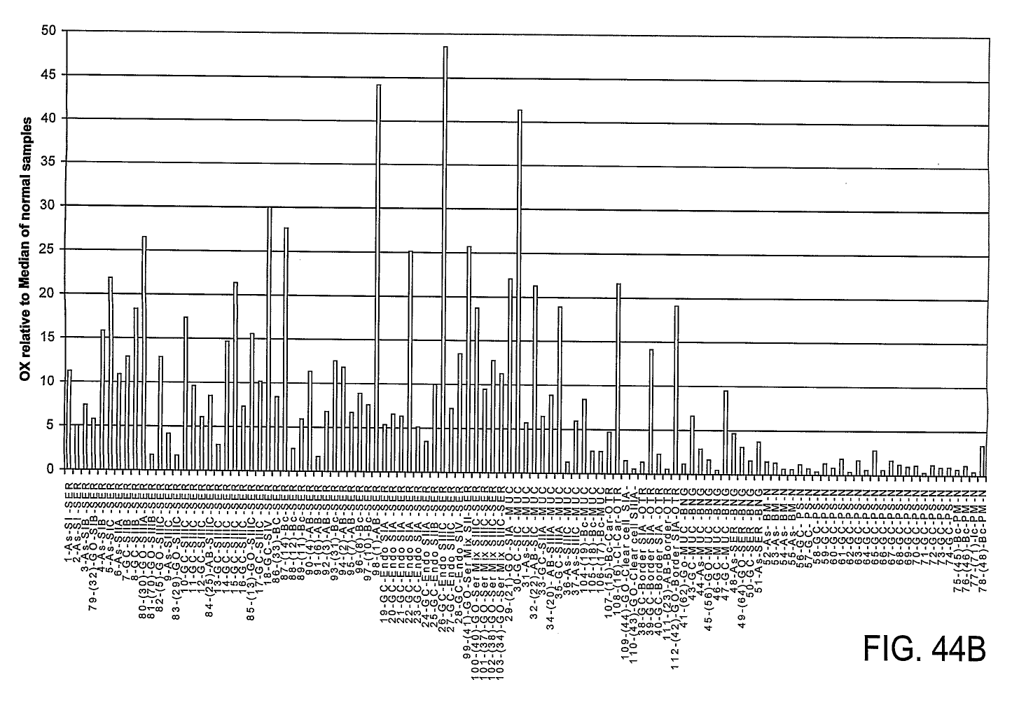

FIG. 44A is a histogram showing over expression of the Homo sapiens immunoglobulin superfamily, member 9 (IGSF9) transcripts detectable by or according to H61775_seg12WT (SEQ ID NO: 270) in cancerous Ovary samples relative to the normal samples.

As is evident from FIG. 44A, the expression of Homo sapiens immunoglobulin superfamily, member 9 (IGSF9) transcripts detectable by the above amplicon in adenocarcinoma samples was higher than in the non-cancerous samples (sample numbers 45, 46, 71 and 48, Table 1_{—}1 above). Notably an over-expression of at least 5 fold was found in 31 out of 43 adenocarcinoma samples, specifically in 23 out of 30 serous carcinoma samples and in 5 out of 6 mucinous carcinoma samples.

Statistical analysis was applied to verify the significance of these results, as described below.

The P value for the difference in the expression levels of Homo sapiens immunoglobulin superfamily, member 9 (IGSF9) transcripts detectable by the above amplicon in Ovary adenocarcinoma samples versus the normal tissue samples was determined by T test as 4.56e-003. The P value for the difference in the expression levels of Homo sapiens immunoglobulin superfamily, member 9 (IGSF9) transcripts detectable by the above amplicon in Ovary serous carcinoma samples versus the normal tissue samples was determined by T test as 1.96e-003.

The above values demonstrate statistical significance of the results.

Primer pairs are also optionally and preferably encompassed within the present invention; for example, for the above experiment, the following primer pair was used as a non-limiting illustrative example only of a suitable primer pair: H61775_seg12WTF (SEQ ID NO: 268) forward primer; and H61775_seg12WTR (SEQ ID NO: 269) reverse primer.

The present invention also preferably encompasses any amplicon obtained through the use of any suitable primer pair; for example, for the above experiment, the following amplicon was obtained as a non-limiting illustrative example only of a suitable amplicon: H61775_seg12WT (SEQ ID NO: 270).

Expression of sapiens immunoglobulin superfamily (SEQ ID NO:350), member 9 (IGSF9) transcripts detectable by or according to seg12WT—H61775_seg12WT (SEQ ID NO: 270) amplicon and primers H61775_seg12WTF (SEQ ID NO: 268) and H61775_seg12WTR (SEQ ID NO: 269) was further measured by real time PCR using enlarged panel as shown in Table 1_{—}5. In parallel the expression of four housekeeping genes —SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)), G6PD (GenBank Accession No. NM_{—}000402 (SEQ ID NO:339); G6PD amplicon (SEQ ID NO: 311)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above), to obtain a value of fold up-regulation for each sample relative to the median of the normal samples.

As is evident from FIG. 44B, the expression of sapiens immunoglobulin superfamily (SEQ ID NO:350), member 9 (IGSF9) transcripts detectable by the above amplicon in adenocarcinoma samples was significantly higher than in the non-cancerous samples (sample numbers 41, 43-78, Table 1_{—}5 above). Notably an over-expression of at least 5 fold was found in 58 out of 74 adenocarcinoma samples, specifically in 32 out of 38 serous carcinoma samples, in 9 out of 10 endometroid-type adenocarcinoma samples and in 9 out of 12 mucinous carcinoma samples.

Statistical analysis was applied to verify the significance of these results, as described below.

The P value for the difference in the expression levels of sapiens immunoglobulin superfamily (SEQ ID NO:350), member 9 (IGSF9) transcripts detectable by the above amplicon in Ovary adenocarcinoma samples versus the non-cancerous tissue samples was determined by T test as 6.25e-13.

Threshold of 5 fold over expression was found to differentiate between adenocarcinoma and non-cancerous samples with P value of 3.33e-14 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H61775_seg12WTF (SEQ ID NO: 268) forward primer; and H61775_seg12WTR (SEQ ID NO: 269) reverse primer.

The present invention also preferably encompasses any amplicon obtained through the use of any suitable primer pair; for example, for the above experiment, the following amplicon was obtained as a non-limiting illustrative example only of a suitable amplicon: H61775_seg12WT (SEQ ID NO: 270).

Expression of Homo sapiens Immunoglobulin Superfamily, Member 9 (IGSF9) H61775 Transcripts which are Detectable by Amplicon as Depicted in Sequence Name H61775_seg12WT (SEQ ID NO: 270) in Normal and Cancerous Lung Tissues

Expression of Homo sapiens immunoglobulin superfamily, member 9 (IGSF9) transcripts detectable by or according to seg12WT—H61775_seg12WT (SEQ ID NO: 270) amplicon and primers H61775_seg12WTF (SEQ ID NO: 268) and H61775_seg12WTR (SEQ ID NO: 269) was measured by real time PCR. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 47, 48, 49, 50, 90, 91, 92, 93, 96, 97, 98 and 99, Table 1_{—}2 above), to obtain a value of fold up-regulation for each sample relative to the median of the normal PM samples.

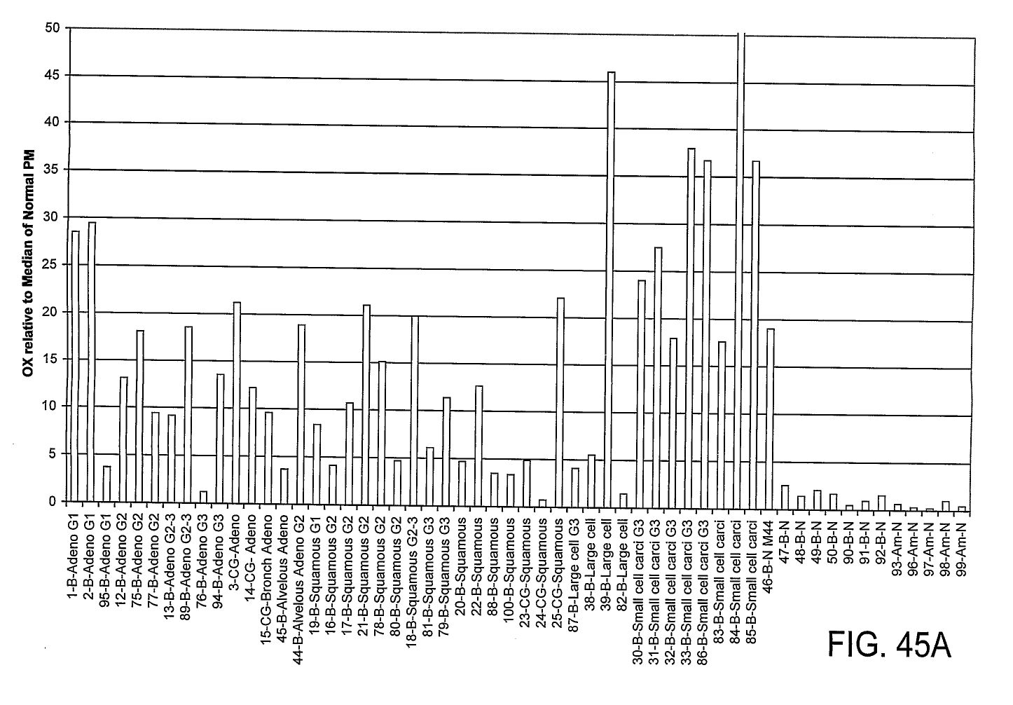

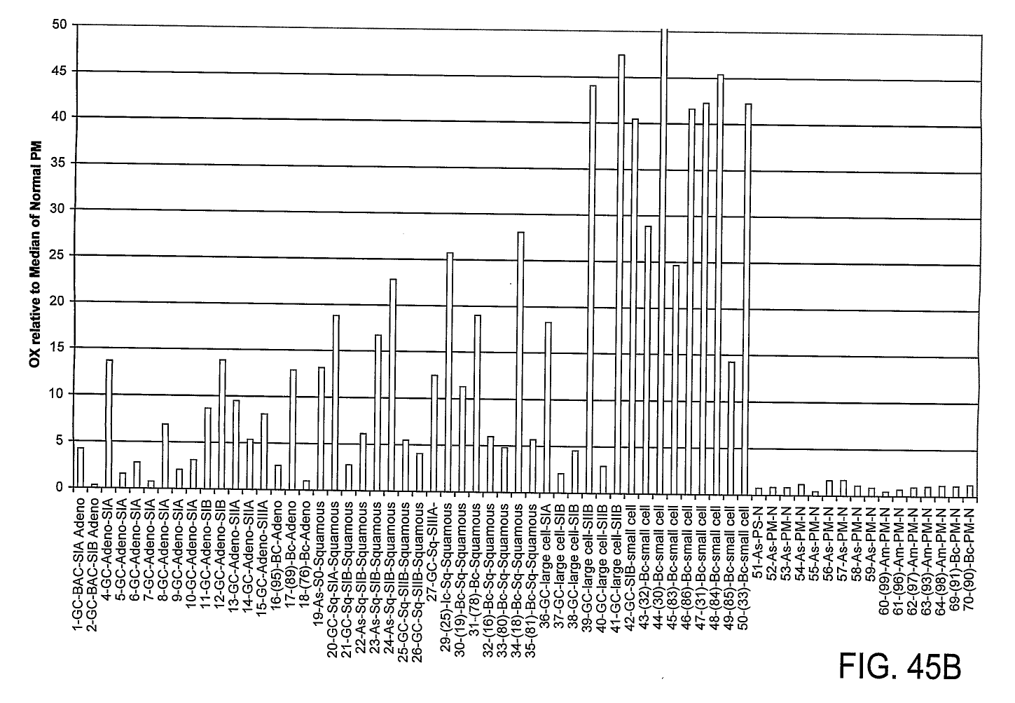

FIG. 45A is a histogram showing over expression of the Homo sapiens immunoglobulin superfamily, member 9 (IGSF9) transcripts detectable by or according to H61775_seg12WT (SEQ ID NO: 270) in cancerous Lung samples relative to the normal samples.

As is evident from FIG. 45A, the expression of Homo sapiens immunoglobulin superfamily, member 9 (IGSF9) transcripts detectable by the above amplicon in cancerous samples was significantly higher than in the non-cancerous samples (sample numbers 47, 48, 49, 50, 90, 91, 92, 93, 96, 97, 98 and 99, Table 1_{—}2 above). Notably an over-expression of at least 5 fold was found in 23 out of 35 non-small cell carcinoma samples, specifically in 12 out of 15 adenocarcinoma samples, in 9 out of 16 squamous cell carcinoma samples in 2 out of 4 large cell carcinoma samples. Also 8 out of 8 small cell carcinoma samples showed over expression of at least 5 fold.

Statistical analysis was applied to verify the significance of these results, as described below.

The P value for the difference in the expression levels of Homo sapiens immunoglobulin superfamily, member 9 (IGSF9) transcripts detectable by the above amplicon in Lung non-small cell carcinoma samples versus the normal tissue samples was determined by T test as 1.43e-007. The P value for the difference in the expression levels of Homo sapiens immunoglobulin superfamily, member 9 (IGSF9) transcripts detectable by the above amplicon in Lung adenocarcinoma samples versus the normal tissue samples was determined by T test as 3.82e-005. The P value for the difference in the expression levels of Homo sapiens immunoglobulin superfamily, member 9 (IGSF9) transcripts detectable by the above amplicon in Lung squamous cell carcinoma samples versus the normal tissue samples was determined by T test as 1.79e-004. The P value for the difference in the expression levels of Homo sapiens immunoglobulin superfamily, member 9 (IGSF9) transcripts detectable by the above amplicon in Lung small cell carcinoma samples versus the normal tissue samples was determined by T test as 1.77e-003.

Threshold of 5 fold over expression was found to differentiate between non-small cell carcinoma and normal samples with P value of 5.18e-005 as checked by exact Fisher test. Threshold of 5 fold over expression was found to differentiate between adenocarcinoma and normal samples with P value of 2.62e-005 as checked by exact Fisher test. Threshold of 5 fold over expression was found to differentiate between squamous cell carcinoma and normal samples with P value of 1.66e-003 as checked by exact Fisher test. Threshold of 5 fold over expression was found to differentiate between small cell carcinoma and normal samples with P value of 7.94e-006 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H61775_seg12WTF (SEQ ID NO: 268) forward primer; and H61775_seg12WTR (SEQ ID NO: 269) reverse primer.

The present invention also preferably encompasses any amplicon obtained through the use of any suitable primer pair; for example, for the above experiment, the following amplicon was obtained as a non-limiting illustrative example only of a suitable amplicon: H61775_seg12WT (SEQ ID NO: 270).

Expression of Homo sapiens immunoglobulin superfamily, member 9 (HGSF9) transcripts detectable by or according to seg12WT-H61775_seg12WT (SEQ ID NO: 270) amplicon and primers H61775_seg12WTF (SEQ ID NO: 268) and H61775_seg12WTR (SEQ ID NO: 269) was further measured by real time PCR using enlarged panel as shown in Table 1_{—}6. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the samples (sample numbers 51-64, 69 and 70, Table 1_{—}6 above), to obtain a value of fold up-regulation for each sample relative to the median of the normal samples

FIG. 45B is a histogram showing over expression of the above-indicated Homo sapiens immunoglobulin superfamily, member 9 (IGSF9) transcripts in cancerous Lung samples relative to the normal samples.

As is evident from FIG. 45B, the expression of Homo sapiens immunoglobulin superfamily, member 9 (IGSF9) transcripts detectable by the above amplicon in small cell carcinoma and non-small cell carcinoma samples was significantly higher than in the non-cancerous samples (sample numbers 51-64, 69 and 70, Table 1_{—}6 above). Notably an over-expression of at least 5 fold was found in 24 out of 39 non-small cell carcinoma samples, specifically in 8 out of 17 adenocarcinoma samples, in 13 out of 16 squamous cell carcinoma samples, and in 3 out of 6 large cell carcinoma samples, and also in 9 out of 9 small cell carcinoma samples and

Statistical analysis was applied to verify the significance of these results, as described below.

The P value for the difference in the expression levels of Homo sapiens immunoglobulin superfamily, member 9 (IGSF9) transcripts detectable by the above amplicon in Lung non-small cell carcinoma samples versus the normal tissue samples was determined by T test as 1.16e-06. The P value for the difference in the expression levels of Homo sapiens immunoglobulin superfamily, member 9 (IGSF9) transcripts detectable by the above amplicon in Lung adenocarcinoma samples versus the normal tissue samples was determined by T test as 7.20e-04. The P value for the difference in the expression levels of Homo sapiens immunoglobulin superfamily, member 9 (IGSF9) transcripts detectable by the above amplicon in Lung squamous cell carcinoma samples versus the normal tissue samples was determined by T test as 4.39e-05. The P value for the difference in the expression levels of Homo sapiens immunoglobulin superfamily, member 9 (IGSF9) transcripts detectable by the above amplicon in Lung small cell carcinoma samples versus the normal tissue samples was determined by T test as 4.10e-05.

Threshold of 5 fold over expression was found to differentiate between non-small cell carcinoma and normal samples with P value of 1.01e-05 as checked by exact Fisher test. Threshold of 5 fold over expression was found to differentiate between adenocarcinoma and normal samples with P value of 1.75e-0 as checked by exact Fisher test. Threshold of 5 fold over expression was found to differentiate between squamous cell carcinoma and normal samples with P value of 1.61e-06 as checked by exact Fisher test. Threshold of 5 fold over expression was found to differentiate between large cell carcinoma and normal samples with P value of 1.30e-02 as checked by exact Fisher test. Threshold of 5 fold over expression was found to differentiate between small cell carcinoma and normal samples with P value of 4.89e-07 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H61775_seg12WTF (SEQ ID NO: 268) forward primer; and H61775_seg12WTR (SEQ ID NO: 269) reverse primer.

The present invention also preferably encompasses any amplicon obtained through the use of any suitable primer pair; for example, for the above experiment, the following amplicon was obtained as a non-limiting illustrative example only of a suitable amplicon: H61775_seg12WT (SEQ ID NO: 270).

Expression of Homo sapiens Immunoglobulin Superfamily, Member 9 (IGSF9) H61775 Transcripts which are Detectable by Amplicon as Depicted in Sequence Name H61775_seg12WT (SEQ ID NO: 270) in Normal and Cancerous Breast Tissues

Expression of Homo sapiens immunoglobulin superfamily, member 9 (IGSF9) transcripts detectable by or according to seg12WT—H61775_seg12WT (SEQ ID NO: 270) amplicon and primers H61775_seg12WTF (SEQ ID NO: 268) and H61775_seg12WTR (SEQ ID NO: 269) was measured by real time PCR. In parallel the expression of four housekeeping genes—G6PD (GenBank Accession No. NM_{—}000402 (SEQ ID NO:339); G6PD amplicon (SEQ ID NO: 311)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 57, 59, 60, 63, 66, 64, 56, 65, 67 and 58, Table 1_{—}3 above), to obtain a value of fold up-regulation for each sample relative to the median of the normal PM samples.

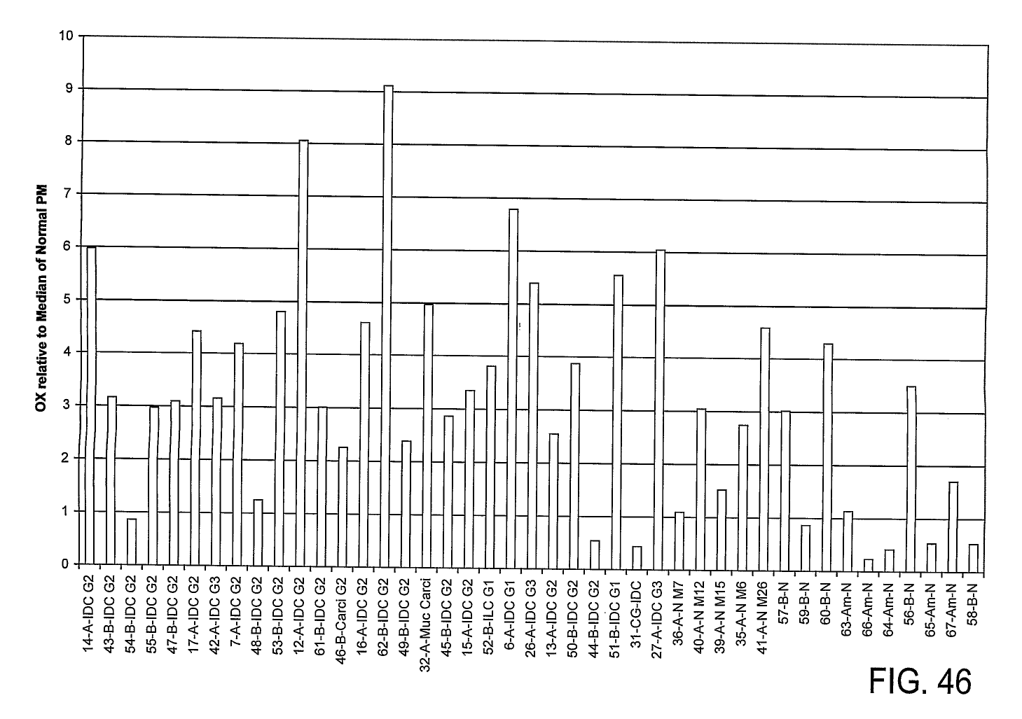

FIG. 46 is a histogram showing over expression of the Homo sapiens immunoglobulin superfamily, member 9 (IGSF9) transcripts detectable by or according to H61775_seg12WT (SEQ ID NO: 270) in cancerous Breast samples relative to the normal samples.

As is evident from FIG. 46, the expression of Homo sapiens immunoglobulin superfamily, member 9 (IGSF9) transcripts detectable by the above amplicon in cancer samples was higher than in the non-cancerous samples (sample numbers 57, 59, 60, 63, 66, 64, 56, 65, 67 and 58, Table 1_{—}3 above). Notably an over-expression of at least 5 fold was found in 7 out of 28 adenocarcinoma samples.

Statistical analysis was applied to verify the significance of these results, as described below.

The P value for the difference in the expression levels of Homo sapiens immunoglobulin superfamily, member 9 (IGSF9) transcripts detectable by the above amplicon in Breast cancer samples versus the normal tissue samples was determined by T test as 1.06e-003.

The above values demonstrate statistical significance of the results.

Primer pairs are also optionally and preferably encompassed within the present invention; for example, for the above experiment, the following primer pair was used as a non-limiting illustrative example only of a suitable primer pair: H61775_seg12WTF (SEQ ID NO: 268) forward primer; and H61775_seg12WTR (SEQ ID NO: 269) reverse primer.

The present invention also preferably encompasses any amplicon obtained through the use of any suitable primer pair; for example, for the above experiment, the following amplicon was obtained as a non-limiting illustrative example only of a suitable amplicon: H61775_seg12WT (SEQ ID NO: 270).

Expression of Homo sapiens Immunoglobulin Superfamily, Member 9 (IGSF9) H61775 Transcripts which are Detectable by Amplicon as Depicted in Sequence Name H61775_seg12WT (SEQ ID NO: 270) in Different Normal Tissues

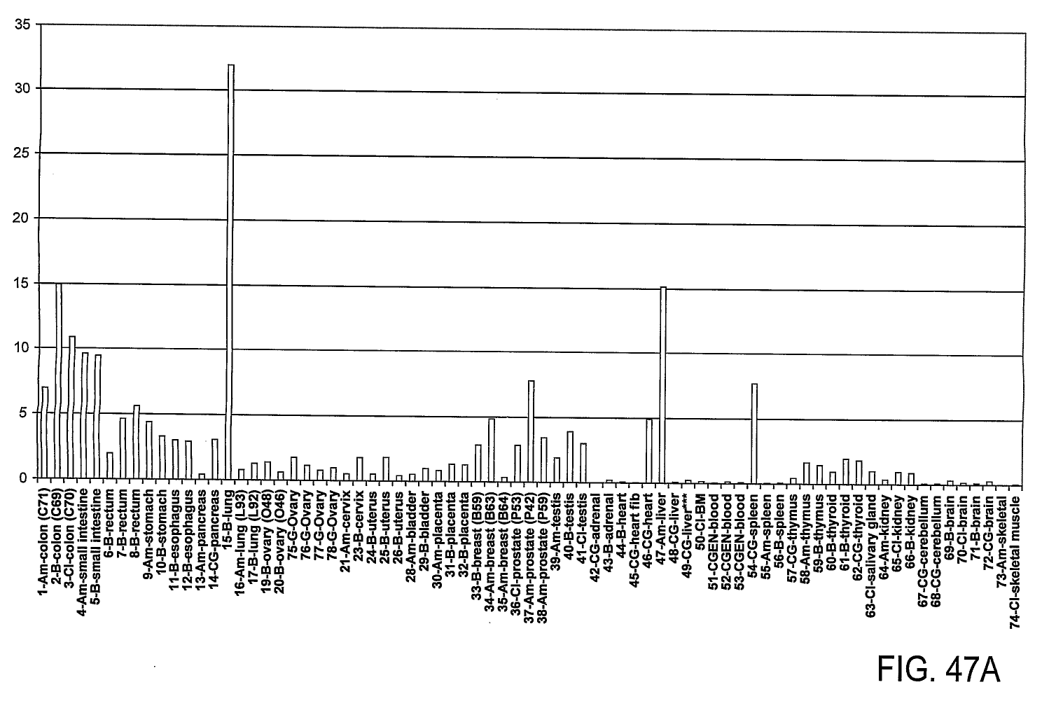

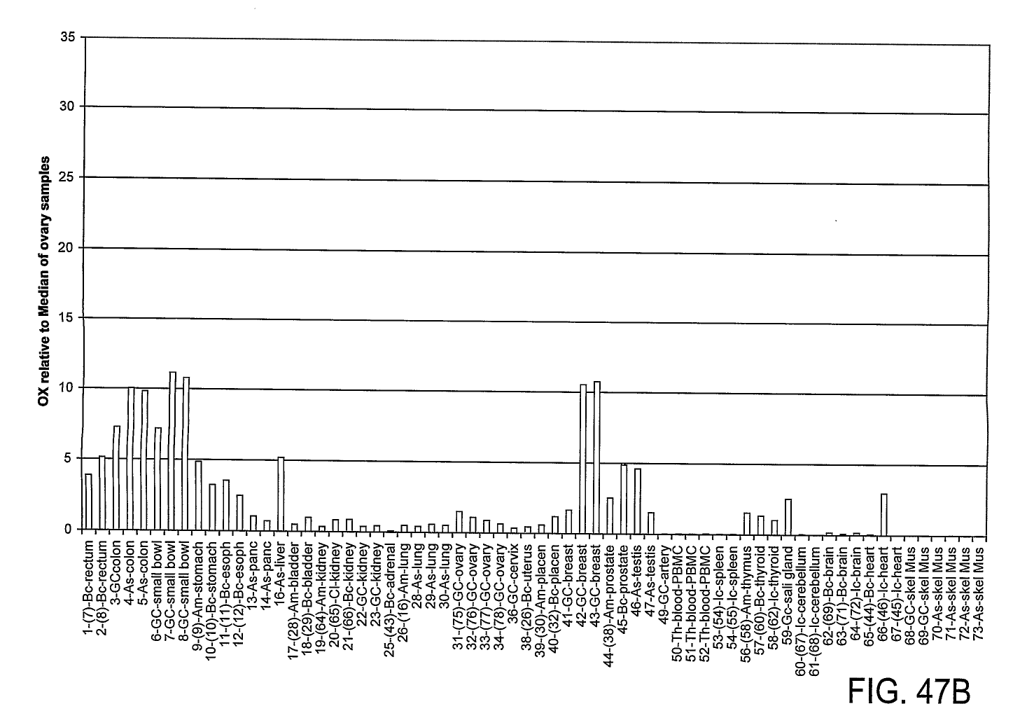

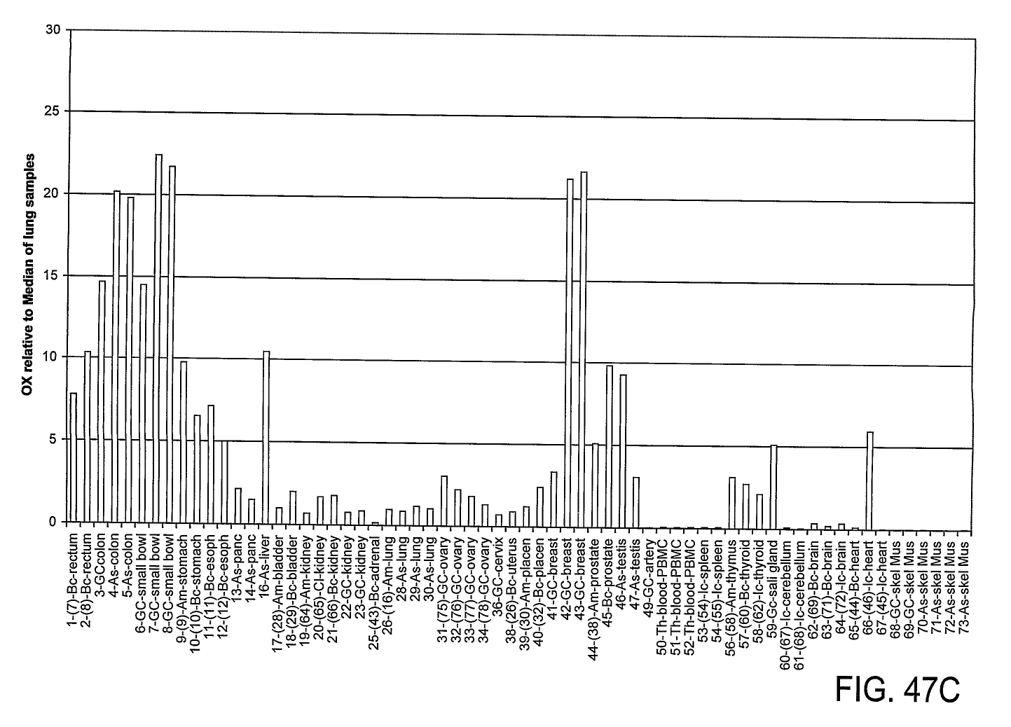

Expression of Homo sapiens immunoglobulin superfamily, member 9 (IGSF9) transcripts detectable by or according to seg12WT-H61775_seg12WT (SEQ ID NO: 270) amplicon and primers H61775_seg12WTF (SEQ ID NO: 268) and H61775_seg12WTR (SEQ ID NO: 269) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the geometric mean of the quantities of the housekeeping genes. The normalized quantity of each RT sample was then divided by the median of the quantities of the ovary samples (sample numbers 19 and 20, Table 1_{—}9 above), to obtain a value of relative expression of each sample relative to the median of the ovary samples. FIG. 47A shows expression of Homo sapiens immunoglobulin superfamily, member 9 (IGSF9) H61775 transcripts which are detectable by amplicon as depicted in sequence name H61775_seg12WT (SEQ ID NO: 270) in different normal tissues.

Expression of Homo sapiens immunoglobulin superfamily, member 9 (IGSF9) transcripts detectable by or according to seg12WT-H61775_seg12WT (SEQ ID NO: 270) amplicon and primers H61775_seg12WTF (SEQ ID NO: 268) and H61775_seg12WTR (SEQ ID NO: 269) was further measured by real time PCR on an updated normal panel, as shown in Table 1_{—}10. In parallel the expression of three housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), and TATA box (GenBank Accession No. NM_{—}003194 (SEQ ID NO:343); TATA amplicon (SEQ ID NO: 323)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the ovary samples (sample numbers 31, 32, 33 and 34, Table 1_{—}10 above), to obtain a value of relative expression of each sample relative to the median of the ovary samples—as presented in FIG. 47B, or by the median of the quantities of the lung samples (sample numbers 26, 28-30, Table 1_{—}10 above), to obtain a value of relative expression of each sample relative to the median of the lung samples—as presented in FIG. 47C.

Expression of Homo sapiens Immunoglobulin Superfamily, Member 9 (IGSF9) H61775 Transcripts which are Detectable by Amplicon as Depicted in Sequence Name H61775_seg8F2R2 (SEQ ID NO: 267) in Normal and Cancerous Lung Tissues

Expression of Homo sapiens immunoglobulin superfamily, member 9 (IGSF9) transcripts detectable by or according to seg8F2R2—H61775_seg8F2R2 (SEQ ID NO: 267) amplicon and primers H61775_seg8F2 (SEQ ID NO: 265) and H61775_seg8R2 (SEQ ID NO: 266) was measured by real time PCR. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BCO 9323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the samples (sample numbers 51-64, 69 and 70, Table 1_{—}6 above), to obtain a value of fold up-regulation for each sample relative to the median of the normal samples.

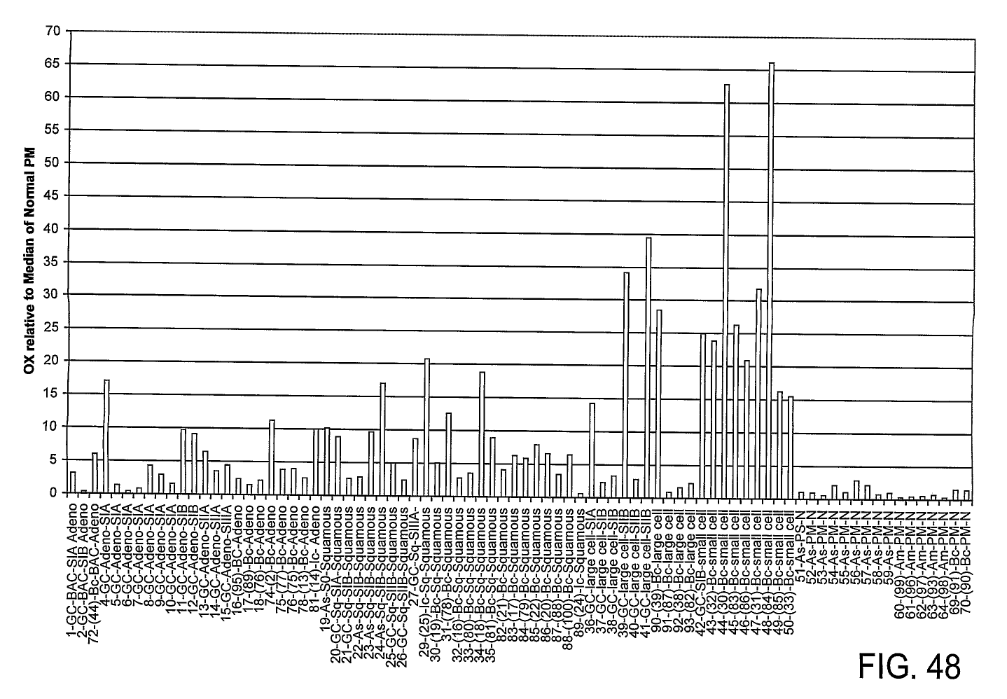

FIG. 48 is a histogram showing over expression of the above-indicated Homo sapiens immunoglobulin superfamily, member 9 (IGSF9) transcripts in cancerous Lung samples relative to the normal samples.

As is evident from FIG. 48, the expression of Homo sapiens immunoglobulin superfamily, member 9 (IGSF9) transcripts detectable by the above amplicon in small cell carcinoma and non-small cell carcinoma samples was significantly higher than in the non-cancerous samples (sample numbers 51-64, 69 and 70, Table 1_{—}6 above). Notably an over-expression of at least 5 fold was found in 25 out of 53 non-small cell carcinoma samples, specifically in 7 out of 23 adenocarcinoma samples, in 15 out of 24 squamous cell carcinoma samples and in 4 out of 10 large cell carcinoma samples, and also in 9 out of 9 small cell carcinoma samples.

Statistical analysis was applied to verify the significance of these results, as described below.

The P value for the difference in the expression levels of Homo sapiens immunoglobulin superfamily, member 9 (IGSF9) transcripts detectable by the above amplicon in all Lung non-small cell carcinoma samples versus the normal tissue samples was determined by T test as 1.42e-07. The P value for the difference in the expression levels of Homo sapiens immunoglobulin superfamily, member 9 (IGSF9) transcripts detectable by the above amplicon in Lung adenocarcinoma samples versus the normal tissue samples was determined by T test as 4.81e-04. The P value for the difference in the expression levels of Homo sapiens immunoglobulin superfamily, member 9 (IGSF9) transcripts detectable by the above amplicon in Lung squamous cell carcinoma samples versus the normal tissue samples was determined by T test as 2.30e-06. The P value for the difference in the expression levels of Homo sapiens immunoglobulin superfamily, member 9 (IGSF9) transcripts detectable by the above amplicon in Lung large cell carcinoma samples versus the normal tissue samples was determined by T test as 3.66e-02. The P value for the difference in the expression levels of Homo sapiens immunoglobulin superfamily, member 9 (IGSF9) transcripts detectable by the above amplicon in Lung small cell carcinoma samples versus the normal tissue samples was determined by T test as 1.24e-03.

Threshold of 5 fold over expression was found to differentiate between all non-small cell carcinoma and normal samples with P value of 2.85e-04 as checked by exact Fisher test. Threshold of 5 fold over expression was found to differentiate between adenocarcinoma and normal samples with P value of 1.59e-02 as checked by exact Fisher test. Threshold of 5 fold over expression was found to differentiate between squamous cell carcinoma and normal samples with P value of 3.25e-05 as checked by exact Fisher test. Threshold of 5 fold over expression was found to differentiate between large cell carcinoma and normal samples with P value of 1.40e-02 as checked by exact Fisher test. Threshold of 5 fold over expression was found to differentiate between small cell carcinoma and normal samples with P value of 4.89e-07 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H61775_seg8F2 (SEQ ID NO: 265) forward primer; and H61775_seg8R2 (SEQ ID NO: 266) reverse primer.

The present invention also preferably encompasses any amplicon obtained through the use of any suitable primer pair; for example, for the above experiment, the following amplicon was obtained as a non-limiting illustrative example only of a suitable amplicon: H61775_seg8F2R2 (SEQ ID NO: 267).

Expression of Homo sapiens Immunoglobulin Superfamily, Member 9 (IGSF9) H61775 Transcripts which are Detectable by Amplicon as Depicted in Sequence Name H61775_seg8F2R2 (SEQ ID NO: 267) in Different Normal Tissues

Expression of Homo sapiens immunoglobulin superfamily, member 9 (IGSF9) transcripts detectable by or according to seg8F2R2—H61775_seg8F2R (SEQ ID NO: 267) amplicon and primers H61775_seg8F2 (SEQ ID NO: 265) and H61775_seg8R2 (SEQ ID NO: 266) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), and TATA box (GenBank Accession No. NM_{—}003194 (SEQ ID NO:343); TATA amplicon (SEQ ID NO: 323)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the ovary samples (sample numbers 31, 32, 33 and 34, Table 1_{—}9 above), to obtain a value of relative expression of each sample relative to the median of the ovary samples.

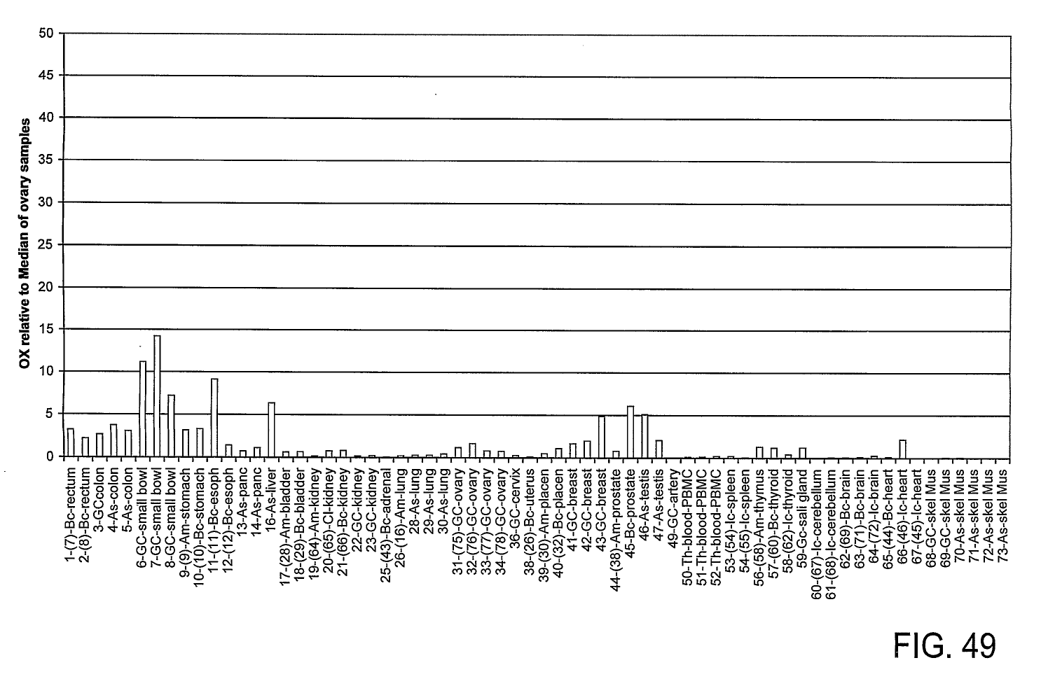

FIG. 49 demonstrates the Expression of Homo sapiens immunoglobulin superfamily, member 9 (IGSF9) H61775 transcripts which are detectable by amplicon as depicted in sequence name H61775_seg8F2R2 (SEQ ID NO: 267) in different normal tissues.

Expression of Sapiens Immunoglobulin Superfamily (SEQ ID NO:350), Member 9 (IGSF9) H61775 Transcripts which are Detectable by Amplicon as Depicted in Sequence Name H61775_seg8F2R2 (SEQ ID NO: 267) in Normal and Cancerous Ovary Tissues

Expression of sapiens immunoglobulin superfamily (SEQ ID NO:350), member 9 (IGSF9) transcripts detectable by or according to seg8F2R2—H61775_seg8F2R2 (SEQ ID NO: 267) amplicon and primers H61775_seg8F2 (SEQ ID NO: 265) and H61775_seg8R2 (SEQ ID NO: 266) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)), G6PD (GenBank Accession No. NM_{—}000402 (SEQ ID NO:339); G6PD amplicon (SEQ ID NO: 311)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above), to obtain a value of fold up-regulation for each sample relative to the median of the normal samples.

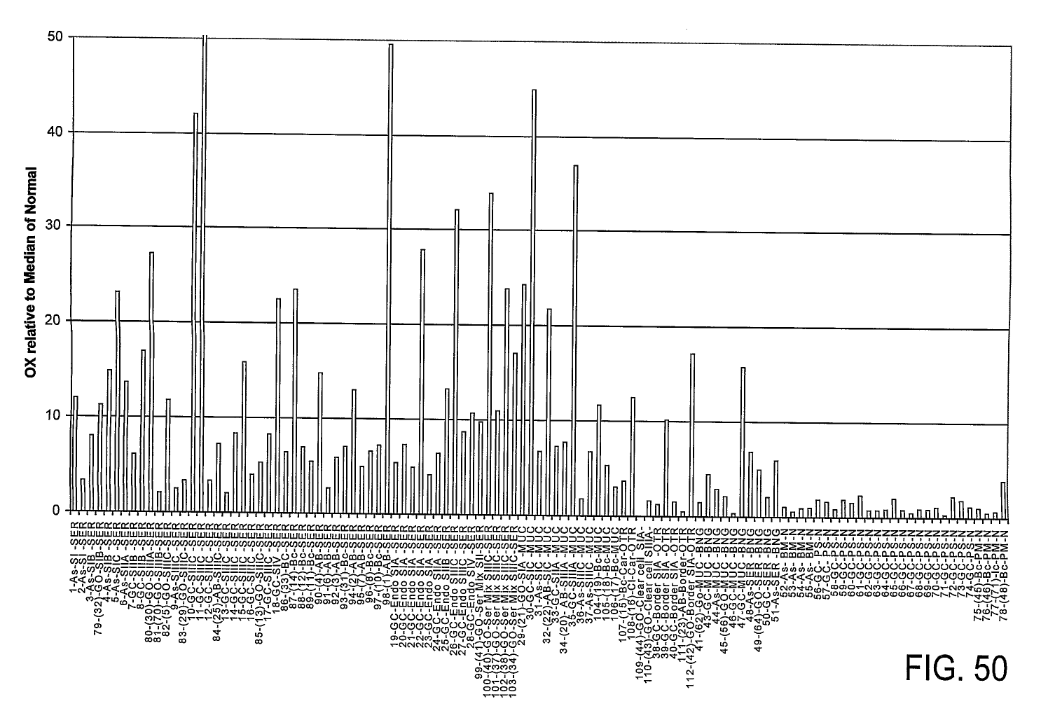

FIG. 50 is a histogram showing over expression of the sapiens immunoglobulin superfamily (SEQ ID NO:350), member 9 (IGSF9) transcripts in cancerous Ovary samples relative to the non-cancerous samples.

As is evident from FIG. 50, the expression of sapiens immunoglobulin superfamily (SEQ ID NO:350), member 9 (IGSF9) transcripts detectable by the above amplicon in adenocarcinoma samples was significantly higher than in the non-cancerous samples (sample numbers 41, 43-78, Table 1_{—}5 above). Notably an over-expression of at least 5 fold was found in 55 out of 74 adenocarcinoma samples, specifically in 29 out of 38 serous carcinoma samples, in 8 out of 10 endometroid-type adenocarcinoma samples and in 10 out of 12 mucinous carcinoma samples.

Statistical analysis was applied to verify the significance of these results, as described below.

The P value for the difference in the expression levels of sapiens immunoglobulin superfamily (SEQ ID NO:350), member 9 (IGSF9) transcripts detectable by the above amplicon in Ovary adenocarcinoma samples versus the non-cancerous tissue samples was determined by T test as 5.44e-09.

Threshold of 5 fold over expression was found to differentiate between adenocarcinoma and non-cancerous samples with P value of 9.77e-12 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H61775_seg8F2 (SEQ ID NO: 265) forward primer; and H61775_seg8R2 (SEQ ID NO: 266) reverse primer.

The present invention also preferably encompasses any amplicon obtained through the use of any suitable primer pair; for example, for the above experiment, the following amplicon was obtained as a non-limiting illustrative example only of a suitable amplicon: H61775_seg8F2R2 (SEQ ID NO: 267).

Expression of Homo sapiens Immunoglobulin Superfamily, Member 9 (IGSF9) H61775 Transcripts which are Detectable by Amplicon as Depicted in Sequence Name H61775_seg8F2R2 (SEQ ID NO: 267) in Normal and Cancerous Colon Tissues

Expression of Homo sapiens immunoglobulin superfamily, member 9 (IGSF9) transcripts detectable by or according to seg8F2R2, H61775_seg8F2R2 (SEQ ID NO: 267) amplicon and primers H61775_seg8F2 (SEQ ID NO: 265) H61775_seg8R2 (SEQ ID NO: 266) was measured by real time PCR. In parallel the expression of three housekeeping genes—PBGD (GenBank Accession No. BC019323 (SEQ ID NO:336); amplicon—PBGD-amplicon (SEQ ID NO: 302)), HPRT1 (GenBank Accession No. NM_{—}000194 (SEQ ID NO:337); amplicon—HPRT1-amplicon (SEQ ID NO: 305) (SEQ ID NO: 305)) and G6PD (GenBank Accession No. NM_{—}000402 (SEQ ID NO:339); G6PD amplicon (SEQ ID NO: 311)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (Sample Nos 52, 42, 43, 44, 45, 46, 47, 48, 49, 50, 51, 53, 54, 55, 56, 57, 58, 59, 60, 61, 62, 63 and 64, Table 1_{—}8, above), to obtain a value of fold differential expression for each sample relative to the median of the normal samples.

In one experiment that was carried out no differential expression in the cancerous samples relative to the normal samples was observed.

Primer pairs are also optionally and preferably encompassed within the present invention; for example, for the above experiment, the following primer pair was used as a non-limiting illustrative example only of a suitable primer pair: H61775_seg8F2 (SEQ ID NO: 265) forward primer; and H61775_seg8R2 (SEQ ID NO: 266) reverse primer.

The present invention also preferably encompasses any amplicon obtained through the use of any suitable primer pair; for example, for the above experiment, the following amplicon was obtained as a non-limiting illustrative example only of a suitable amplicon: H61775_seg8F2R2 (SEQ ID NO: 267).

Expression of Homo sapiens Immunoglobulin Superfamily, Member 9 (IGSF9) H61775 Transcripts which are Detectable by Amplicon as Depicted in Sequence Name H61775_seg5-6F1R1 (SEQ ID NO: 394) in Normal and Cancerous Ovary Tissues

Expression of Homo sapiens immunoglobulin superfamily, member 9 (IGSF9) transcripts detectable by or according to seg5-6F1R1—H61775_seg5-6F1R1 (SEQ ID NO: 394) amplicon and primers H61775_seg5-6F (SEQ ID NO: 392) and H61775_seg5-6R (SEQ ID NO: 393) was measured by real time PCR. In parallel the expression of three housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)) and G6PD (GenBank Accession No. NM_{—}000402 (SEQ ID NO:339); G6PD amplicon (SEQ ID NO: 311)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above), to obtain a value of fold up-regulation for each sample relative to the median of the normal samples.

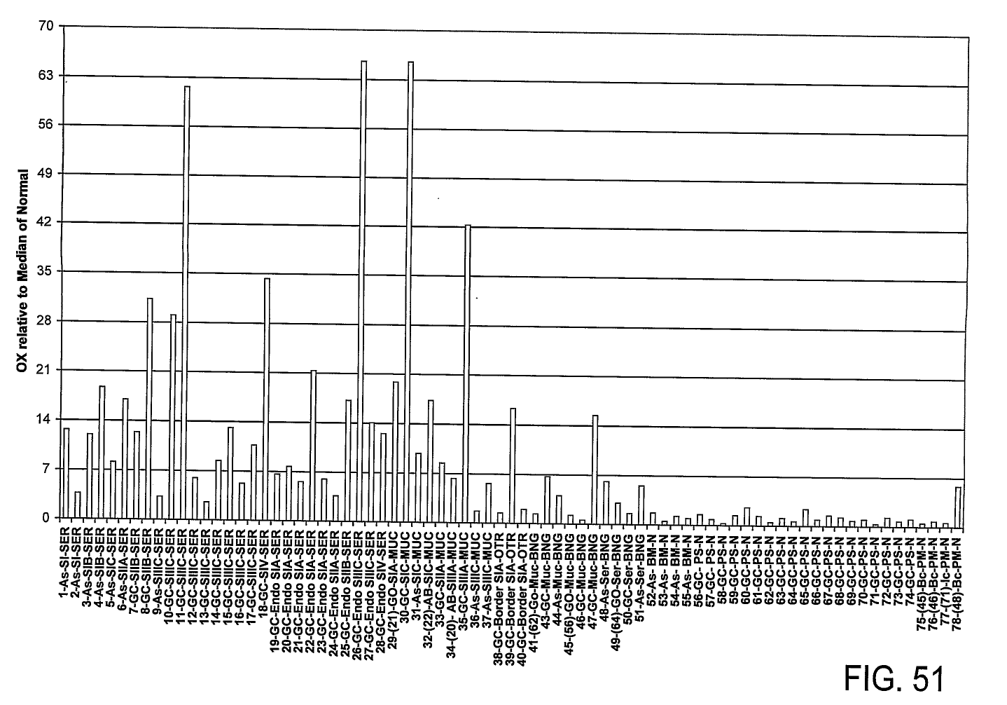

FIG. 51 is a histogram showing over expression of the above-indicated Homo sapiens immunoglobulin superfamily, member 9 (IGSF9) transcripts in cancerous Ovary samples relative to the normal samples.

As is evident from FIG. 51, the expression of Homo sapiens immunoglobulin superfamily, member 9 (IGSF9) transcripts detectable by the above amplicon in adenocarcinoma samples was significantly higher than in the non-cancerous samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above). Notably an over-expression of at least 7 fold was found in 26 out of 40 adenocarcinoma samples, specifically in 13 out of 18 serous carcinoma samples, in 6 out of 9 mucinous carcinoma samples, and in 6 out of 10 endometroid samples.

Statistical analysis was applied to verify the significance of these results, as described below.

The P value for the difference in the expression levels of Homo sapiens immunoglobulin superfamily, member 9 (IGSF9) transcripts detectable by the above amplicon in Ovary adenocarcinoma samples versus the normal tissue samples was determined by T test as 1.59e-006. The P value for the difference in the expression levels of Homo sapiens immunoglobulin superfamily, member 9 (IGSF9) transcripts detectable by the above amplicon in Ovary serous carcinoma samples versus the normal tissue samples was determined by T test as 5.56e-04. The P value for the difference in the expression levels of Homo sapiens immunoglobulin superfamily, member 9 (IGSF9) transcripts detectable by the above amplicon in Ovary mucinous carcinoma samples versus the normal tissue samples was determined by T test as 3.07e-002. The P value for the difference in the expression levels of Homo sapiens immunoglobulin superfamily, member 9 (IGSF9) transcripts detectable by the above amplicon in Ovary endometroid samples versus the normal tissue samples was determined by T test as 3.16e-002.

Threshold of 7 fold over expression was found to differentiate between adenocarcinoma and normal samples with P value of 8.58e-009 as checked by exact Fisher test. Threshold of 7 fold over expression was found to differentiate between serous carcinoma and normal samples with P value of 1.17e-07 as checked by exact Fisher test. Threshold of 7 fold over expression was found to differentiate between mucinous carcinoma and normal samples with P value of 4.31e-005 as checked by exact Fisher test. Threshold of 7 fold over expression was found to differentiate between endometroid and normal samples with P value of 9.03e-005 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H61775_seg5-6F (SEQ ID NO: 392) forward primer; and H61775_seg5-6R (SEQ ID NO: 393) reverse primer.

The present invention also preferably encompasses any amplicon obtained through the use of any suitable primer pair; for example, for the above experiment, the following amplicon was obtained as a non-limiting illustrative example only of a suitable amplicon: H61775_seg5-6F1R1 (SEQ ID NO: 394).

Expression of Homo sapiens Immunoglobulin Superfamily, Member 9 (IGSF9) H61775 Transcripts which are Detectable by Amplicon as Depicted in Sequence Name H61775_seg5-6 (SEQ ID NO: 394) in Normal and Cancerous Lung Tissues

Expression of Homo sapiens immunoglobulin superfamily, member 9 (IGSF9) transcripts detectable by or according to seg5-6—H61775_seg5-6 (SEQ ID NO: 394) amplicon and primers H61775_seg5-6F (SEQ ID NO: 392) and H61775_seg5-6R (SEQ ID NO: 393) was measured by real time PCR. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 51, 52, 53, 54, 55, 56, 57, 58, 59, 60, 61, 62, 63, 64, 69 and 70, Table 1_{—}6 above), to obtain a value of fold up-regulation for each sample relative to the median of the normal samples.

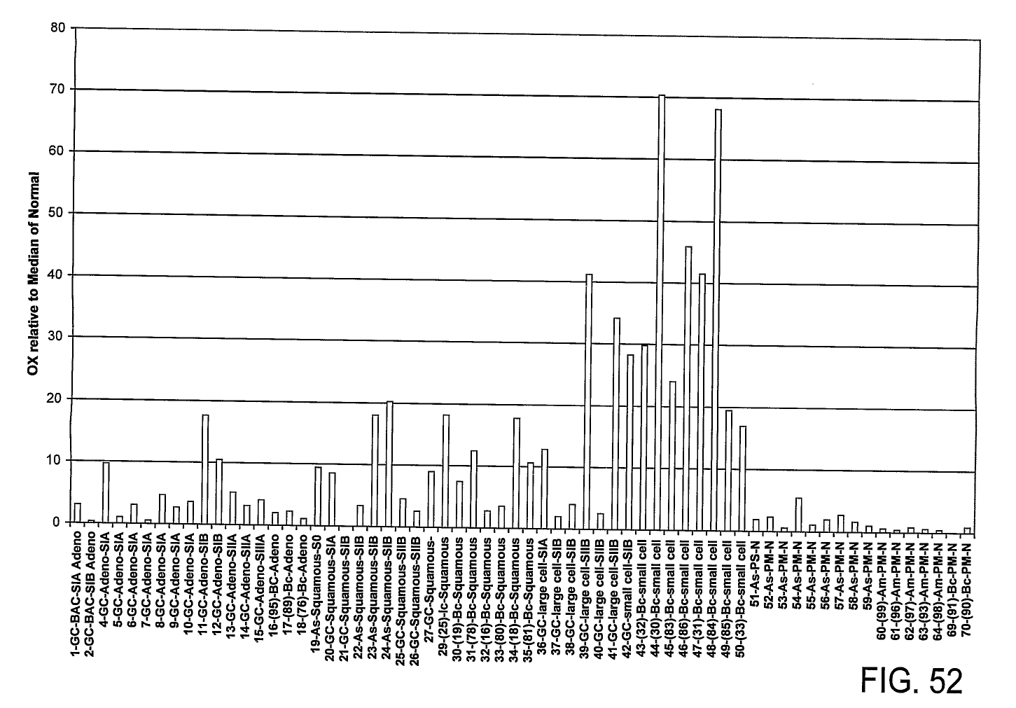

FIG. 52 is a histogram showing over expression of the above-indicated Homo sapiens immunoglobulin superfamily, member 9 (IGSF9) transcripts in cancerous Lung samples relative to the normal samples.

As is evident from FIG. 52, the expression of Homo sapiens immunoglobulin superfamily, member 9 (IGSF9) transcripts detectable by the above amplicon in small cell carcinoma and non-small cell carcinoma samples was significantly higher than in the non-cancerous samples (sample numbers 51, 52, 53, 54, 55, 56, 57, 58, 59, 60, 61, 62, 63, 64, 69 and 70, Table 1_{—}6 above). Notably an over-expression of at least 10 fold was found in 11 out of 39 non-small cell carcinoma samples, specifically in 6 out of 16 squamous cell carcinoma samples, in 3 out of 6 large cell carcinoma samples, and also in 9 out of 9 small cell carcinoma samples.

Statistical analysis was applied to verify the significance of these results, as described below.

The P value for the difference in the expression levels of Homo sapiens immunoglobulin superfamily, member 9 (IGSF9) transcripts detectable by the above amplicon in Lung non-small cell carcinoma samples versus the normal tissue samples was determined by T test as 4.37e-005. The P value for the difference in the expression levels of Homo sapiens immunoglobulin superfamily, member 9 (IGSF9) transcripts detectable by the above amplicon in Lung adenocarcinoma samples versus the normal tissue samples was determined by T test as 1.72e-002. The P value for the difference in the expression levels of Homo sapiens immunoglobulin superfamily, member 9 (IGSF9) transcripts detectable by the above amplicon in Lung squamous cell carcinoma samples versus the normal tissue samples was determined by T test as 2.11e-004. The P value for the difference in the expression levels of Homo sapiens immunoglobulin superfamily, member 9 (IGSF9) transcripts detectable by the above amplicon in Lung small cell carcinoma samples versus the normal tissue samples was determined by T test as 5.35e-004.

Threshold of 10 fold over expression was found to differentiate between non-small cell carcinoma and normal samples with P value of 1.40e-002 as checked by exact Fisher test. Threshold of 10 fold over expression was found to differentiate between squamous cell carcinoma and normal samples with P value of 8.84e-003 as checked by exact Fisher test. Threshold of 10 fold over expression was found to differentiate between large cell carcinoma and normal samples with P value of 1.30e-002 as checked by exact Fisher test. Threshold of 10 fold over expression was found to differentiate between small cell carcinoma and normal samples with P value of 4.89e-007 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: H61775_seg5-6F (SEQ ID NO: 392) forward primer; and H61775_seg5-6R (SEQ ID NO: 393) reverse primer.

The present invention also preferably encompasses any amplicon obtained through the use of any suitable primer pair; for example, for the above experiment, the following amplicon was obtained as a non-limiting illustrative example only of a suitable amplicon: H61775_seg5-6 (SEQ ID NO: 394).

Expression of Homo sapiens Immunoglobulin Superfamily, Member 9 (IGSF9) H61775 Transcripts which are Detectable by Amplicon as Depicted in Sequence Name H61775_seg5-6 (SEQ ID NO: 394) in Different Normal Tissues

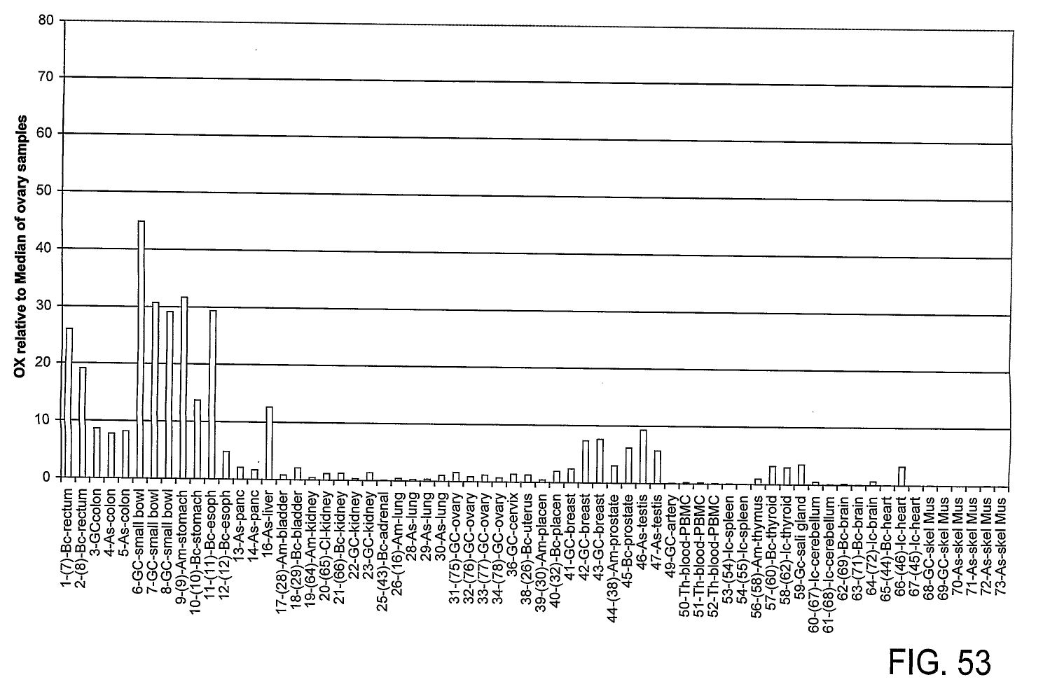

Expression of Homo sapiens immunoglobulin superfamily, member 9 (IGSF9) transcripts detectable by or according to seg5-6—H61775 seg5-6 (SEQ ID NO: 394) amplicon and primers H61775 seg5-6F (SEQ ID NO: 392) and H61775 seg5-6R (SEQ ID NO: 393) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the ovary samples (sample numbers 31-34, Table 1_{—}10 above), to obtain a value of relative expression of each sample relative to the median of the lung samples. FIG. 53 is a histogram showing expression of Homo sapiens immunoglobulin superfamily, member 9 (IGSF9) H61775 transcripts which are detectable by amplicon as depicted in sequence name H61775_seg5-6F1R1 (SEQ ID NO: 394) in different normal tissues.

Expression of Homo sapiens Immunoglobulin Superfamily, member 9 (IGSF9) H61775 Transcripts which are Detectable by Amplicon as Depicted in Sequence Name H61775_seg5-6F1R1 (SEQ ID NO: 394) in Normal and Cancerous Breast Tissues

Expression of Homo sapiens immunoglobulin superfamily, member 9 (IGSF9) transcripts detectable by or according to H61775_seg5-6F1R1 (SEQ ID NO: 394), amplicon and primers H61775_seg5-6F (SEQ ID NO: 392) H61775_seg5-6R (SEQ ID NO: 393) was measured by real time PCR. In parallel the expression of four housekeeping genes—PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)), G6PD (GenBank Accession No. NM_{—}000402 (SEQ ID NO:339); G6PD amplicon (SEQ ID NO: 311)) and RPL19 (GenBank Accession No. NM_{—}000981 (SEQ ID NO:342); RPL19 amplicon (SEQ ID NO: 320)) was measured similarly. For each RT sample, the expression of the above amplicon was normalized to normalization factor calculated from the expression of these house keeping genes as described in normalization method 2 in the “materials and methods” section. The normalized quantity of each RT sample was then divided by the median of the quantities of the normal samples (Sample Nos. 43, 45, 46, 47, 48, 49, 50, 51, 52, 54, 56, 58, 59, 60, 61, 62, 63, 64, 65, 66, 67, 68 and 69, Table 1_{—}7, above), to obtain a value of fold differential expression for each sample relative to the median of the normal samples.

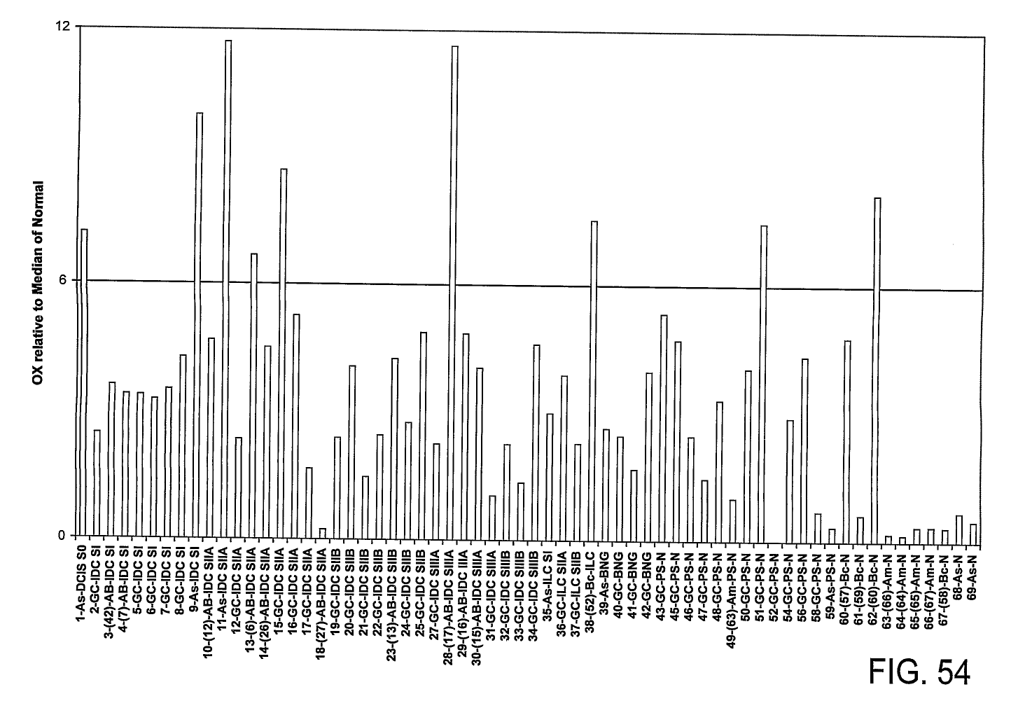

FIG. 54 is a histogram showing low over expression of the above-indicated Homo sapiens immunoglobulin superfamily, member 9 (IGSF9) transcripts in cancerous Breast samples relative to the normal samples.

As is evident from FIG. 54, the expression of Homo sapiens immunoglobulin superfamily, member 9 (IGSF9) transcripts detectable by the above amplicon was higher in several cancer samples than in the non-cancerous samples (sample numbers 39, 41, 42, 43, 45, 46, 47, 48, 49, 50, 51, 52, 54, 56, 58, 59, 60, 61, 62, 63, 64, 65, 66, 67, 68 and 69, Table 1_{—}7 above). Notably an over-expression of at least 5 fold was found in 7 out of 37 adenocarcinoma samples.

Statistical analysis was applied to verify the significance of these results, as described below.

The P value for the difference in the expression levels of Homo sapiens immunoglobulin superfamily, member 9 (IGSF9) transcripts detectable by the above amplicon in Breast cancer samples versus the normal tissue samples was determined by T test as 9.00e-03.

The above values demonstrate statistical significance of the results.

Primer pairs are also optionally and preferably encompassed within the present invention; for example, for the above experiment, the following primer pair was used as a non-limiting illustrative example only of a suitable primer pair: [H61775_seg5-6F (SEQ ID NO: 392) forward primer; and H61775_seg5-6R (SEQ ID NO: 393) reverse primer.

The present invention also preferably encompasses any amplicon obtained through the use of any suitable primer pair; for example, for the above experiment, the following amplicon was obtained as a non-limiting illustrative example only of a suitable amplicon: H61775_seg5-6F1R1 (SEQ ID NO: 394).

### Description for Cluster HUMPAX8A

Cluster HUMPAX8A features 4 transcript(s) and 13 segment(s) of interest, the names for which are given in Tables 94 and 95, respectively, the sequences themselves are given in the “Sequence Listing” of the application. The selected protein variants are given in table 96.

TABLE 94
Transcripts of interest
Transcript Name

HUMPAX8A_T21 (SEQ ID NO: 421)
HUMPAX8A_T24 (SEQ ID NO: 156)
HUMPAX8A_T26 (SEQ ID NO: 157)
HUMPAX8A_T30 (SEQ ID NO: 158)

TABLE 95
Segments of interest
Segment Name

HUMPAX8A_N5 (SEQ ID NO: 159)
HUMPAX8A_N6 (SEQ ID NO: 160)
HUMPAX8A_N9 (SEQ ID NO: 161)
HUMPAX8A_N16 (SEQ ID NO: 162)
HUMPAX8A_N17 (SEQ ID NO: 163)
HUMPAX8A_N18 (SEQ ID NO: 164)
HUMPAX8A_N19 (SEQ ID NO: 165)
HUMPAX8A_N21 (SEQ ID NO: 166)
HUMPAX8A_N0 (SEQ ID NO: 167)
HUMPAX8A_N2 (SEQ ID NO: 168)
HUMPAX8A_N8 (SEQ ID NO: 169)
HUMPAX8A_N13 (SEQ ID NO: 170)
HUMPAX8A_N20 (SEQ ID NO: 171)

TABLE 96
Proteins of interest

| Protein Name | Corresponding Transcript(s) |
|---|---|
| HUMPAX8A_P33 (SEQ ID | HUMPAX8A_T26 (SEQ ID NO: 157) |
| NO: 183) | |
| HUMPAX8A_P36 (SEQ ID | HUMPAX8A_T21 (SEQ ID NO: 421) |
| NO: 184) | |
| HUMPAX8A_P37 (SEQ ID | HUMPAX8A_T24 (SEQ ID NO: 156) |
| NO: 185) | |
| HUMPAX8A_P40 (SEQ ID | HUMPAX8A_T30 (SEQ ID NO: 158) |
| NO: 186) | |

These sequences are variants of the known protein Paired box protein Pax-8 (SEQ ID NO: 172) (SwissProt accession identifier PAX8-HUMAN), referred to herein as the previously known protein.

Protein Paired box protein Pax-8 (SEQ ID NO: 172) is known or believed to have the following function(s): Transcription factor for the thyroid-specific expression of the genes exclusively expressed in the thyroid cell type, maintaining the functional differentiation of such cells. Known polymorphisms for this sequence are as shown in Table 97.

TABLE 97
Amino acid mutations for Known Protein

| SNP position(s) | |
|---|---|
| on amino | |
| acid sequence | Comment |
| 31 | R -> H (in TD; loss of activity). /FTId = VAR_012769 |
| 40 | Q -> P (in TD; loss of activity). /FTId = VAR_012770 |
| 57 | C -> Y (in TD; loss of activity). /FTId = VAR_012771 |
| 62 | L -> R (in TD; loss of activity). /FTId = VAR_012772 |
| 329 | F -> L. /FTId = VAR_012773 |
| 300 | Missing |
| 418 | G -> R |

Protein Paired box protein Pax-8 (SEQ ID NO: 172) localization is believed to be Nuclear.

PCT/US05/03002 and U.S. patent application Ser. No. 11/051,646, filed on Jan. 27, 2005 and assigned to the applicant of the present invention, disclose polynucleotides of HUMPAX8A contig, and assays and methods of use thereof in the diagnosis using NAT based technologies. In this application the HUMPAX8A polynucleotides are described as being overexpressed in epithelial malignant tumors, a mixture of malignant tumors from different tissues, ovarian carcinoma and uterine malignancies.

In addition, HLMPAX8A_P36 was previously disclosed by the inventors in published PCT application no WO2005/071058, hereby incorporated by reference as if fully set forth herein.

Unexpectedly, novel HUMPAX8A variant polynucleotides and their respective encoded polypeptides were identified and are now provided. The novel HUMPAX8A variants are now disclosed to be differentially expressed in ovarian cancer, and therefore may be contemplated within the scope of the present invention as diagnostic markers for this disease. Surprisingly, novel HUMPAX8A variants demonstrated an age related differential diagnostic capability. Also HUMPAX8A_P36 was now surprisingly found by the inventors to be differential diagnostic capability for ovarian cancer and therefore may be contemplated within the scope of the present invention as a diagnostic marker for this disease. Therefore, the present invention further provides diagnostic methods, kits and assays for diagnosis, assessment and prognostic indications regarding to ovarian cancer, according to the age of the subject, as described herein.

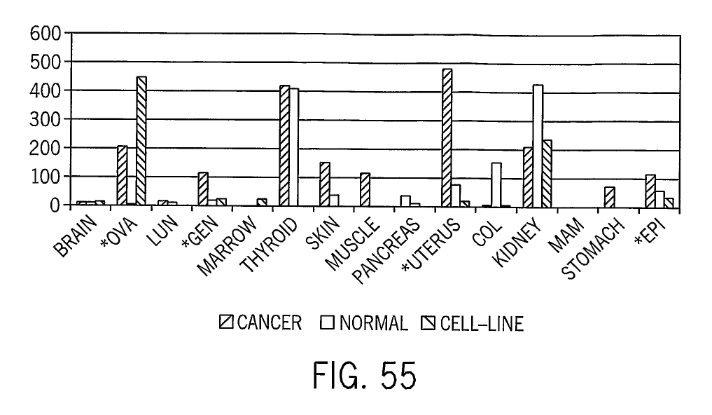

Cluster HUMPAX8A can be used as a diagnostic marker according to overexpression of transcripts of this cluster in cancer. Expression of such transcripts in normal tissues is also given according to the previously described methods. The term “number” in the left hand column of the table and the numbers on the y-axis of FIG. 55 refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 55 and Table 98. This cluster is overexpressed (at least at a minimum level) in the following pathological conditions: ovarian carcinoma, a mixture of malignant tumors from different tissues, uterine malignancies and epithelial malignant tumors.

TABLE 98
Normal tissue distribution

| Name of Tissue | Number |
|---|---|
| brain | 10 |
| ovary | 7 |
| lung | 10 |
| general | 24 |
| bone marrow | 0 |
| Thyroid | 408 |
| skin | 40 |
| muscle | 1 |
| pancreas | 10 |
| uterus | 76 |
| colon | 153 |
| kidney | 425 |
| breast | 0 |
| stomach | 0 |
| epithelial | 61 |

TABLE 99
P values and ratios for expression in cancerous tissue

| Name of Tissue | P1 | P2 | SP1 | R3 | SP2 | R4 |
|---|---|---|---|---|---|---|
| brain | 8.0e−01 | 3.6e−01 | 6.1e−01 | 1.0 | 3.9e−01 | 1.3 |
| ovary | 5.6e−02 | 5.2e−02 | 1.0e−02 | 4.8 | 1.0e−03 | 4.5 |
| lung | 3.0e−01 | 5.1e−01 | 6.5e−01 | 1.4 | 8.5e−01 | 0.9 |
| general | 1.1e−08 | 1.4e−05 | 1.6e−23 | 4.1 | 1.7e−12 | 2.6 |
| bone marrow | N/A | 7.1e−01 | N/A | N/A | 5.4e−01 | 1.9 |
| Thyroid | 3.3e−01 | 3.3e−01 | 6.8e−01 | 0.9 | 6.8e−01 | 0.9 |
| skin | 3.8e−01 | 6.7e−01 | 1.6e−01 | 3.2 | 9.4e−01 | 0.5 |
| muscle | 1.7e−01 | 2.3e−01 | 1.5e−01 | 6.5 | 4.0e−01 | 2.4 |
| pancreas | 2.4e−01 | 3.8e−01 | 2.1e−01 | 2.5 | 3.5e−01 | 1.9 |
| uterus | 1.4e−02 | 1.3e−01 | 1.8e−05 | 3.8 | 9.0e−03 | 2.1 |
| colon | 4.9e−01 | 3.5e−01 | 1.0e+00 | 0.2 | 1.0e+00 | 0.2 |
| kidney | 2.5e−01 | 4.3e−01 | 9.8e−01 | 0.5 | 9.9e−01 | 0.4 |
| breast | 3.6e−01 | 4.8e−01 | N/A | N/A | N/A | N/A |
| stomach | 2.7e−01 | 6.4e−01 | 2.5e−01 | 3.0 | 6.3e−01 | 1.5 |
| epithelial | 4.9e−05 | 6.7e−03 | 6.2e−04 | 1.6 | 1.7e−01 | 1.1 |

As noted above, cluster HUMPAX8A features 4 transcript(s), which were listed in Table 94 above. These transcript(s) encode for protein(s) which are variant(s) of protein Paired box protein Pax-8 (SEQ ID NO: 172). A description of each variant protein according to the present invention is now provided.

Variant protein HUMPAX8A_P33 (SEQ ID NO: 183) according to the present invention has an amino acid sequence as given in the “Sequence Listing” of the application; it is encoded by transcript(s) HUMPAX8A_T26 (SEQ ID NO: 157). An alignment is given to the known protein (Paired box protein Pax-8, SEQ ID NOs:172-182) in the alignment table, on the attached CDROM. One or more alignments to one or more previously published protein sequences are given in the alignment table, on the attached CDROM. A brief description of the relationship of the variant protein according to the present invention to each such aligned protein is as follows:

1. Comparison Report Between HUMPAX8A_P33 (SEQ ID NO: 183) and Q06710-5 (SEQ ID NO: 178) (or NP_{—}054698 (SEQ ID NO: 181), NP_{—}039247 (SEQ ID NO: 180), Q06710-4 (SEQ ID NO: 176), NP_{—}039245 (SEQ ID NO: 179), Q06710-2 (SEQ ID NO: 175), or NP 039246 (SEQ ID NO: 173)):

A. An isolated chimeric polypeptide encoding for HUMPAX8A_P33 (SEQ ID NO: 183), comprising a first amino acid sequence being at least about 90% homologous to MPHNSIRSGHGGLNQLGGAFVNGRPLPEVVRQRIVDLAHQGVRPCDISRQLRVSHGCVSKILGRYYETGSI RPGVIGGSKPKVATPKVVEKIGDYKRQNPTMFAWEIRDRLLAEGVCDNDTVPSVSSINRIIRTKVQQPFNLP MDSCVATKSLSPGHTLIPSSAVTPPESPQSDSLGSTYSINGLLGIAQPGSDKRKMDDS corresponding to amino acids 1-201 of Q06710-5 (SEQ ID NO: 178), which also corresponds to amino acids 1-201 of HUMPAX8A_P33 (SEQ ID NO: 183), and a second amino acid sequence being at least about 70%, optionally at least 80%, preferably at least 85%, more preferably at least 90% and most preferably at least 95% homologous to a polypeptide having the sequence ECWDRRAAGGAREQFAQALMGTETLPLMIWDFVGALHVTVTCPVPQLAKAPGPTWLALFLPPQ (SEQ ID NO: 506) corresponding to amino acids 202-264 of HUMPAX8A_P33 (SEQ ID NO: 183), wherein said first amino acid sequence and second amino acid sequence are contiguous and in a sequential order.

B. An isolated polypeptide encoding for an edge portion of HUMPAX8A_P33 (SEQ ID NO: 183), comprising an amino acid sequence being at least about 70%, optionally at least about 80%, preferably at least about 85%, more preferably at least about 90% and most preferably at least about 95% homologous to the sequence

(SEQ ID NO: 506)

ECWDRRAAGGAREQFAQALMGTETLPLMIWDFVGALHVTVTCPVPQLAKA
PGPTWLALFLPPQ
of

(SEQ ID NO: 183)

HUMPAX8A_P33.

The localization of the variant protein was determined according to results from a number of different software programs and analyses, including analyses from SignalP and other specialized programs. The variant protein is believed to be located intracellularly with regard to the cell.

Variant protein HUMPAX8A_P33 (SEQ ID NO: 183) is encoded by the following transcript(s): HUMPAX8A_T26 (SEQ ID NO: 157), for which coding portion starts at position 168 and ends at position 959. The transcript also has the following SNPs as listed in Table 100 (given according to their position on the nucleotide sequence, with the alternative nucleic acid listed).

TABLE 100
Nucleic acid SNPs

| | SNP position(s) on nucleotide |
|---|---|
| Polymorphism | sequence |
| G -> | 42 |
| A -> G | 4685 |

Variant protein HUMPAX8A_P36 (SEQ ID NO: 184) according to the present invention has an amino acid sequence as given in the “Sequence Listing” of the application; it is encoded by transcript(s) HUMPAX8A_T21 (SEQ ID NO: 421). An alignment is given to the known protein (Paired box protein Pax-8 (SEQ ID NO: 172) SEQ ID NOs:172-182) in the alignment table, on the attached CDROM. One or more alignments to one or more previously published protein sequences are given in the alignment table, on the attached CDROM. A brief description of the relationship of the variant protein according to the present invention to each such aligned protein is as follows:

1. Comparison Report Between HUMPAX8A_P36 (SEQ ID NO: 184) and PAX8_HUMAN (SEQ ID NO:422) (or Q06710-5 (SEQ ID NO: 178), NP_{—}054698 (SEQ ID NO: 181), NP_{—}039247 (SEQ ID NO: 180), Q06710-4 (SEQ ID NO: 176), NP_{—}039245 (SEQ ID NO: 179), Q06710-2 (SEQ ID NO: 175), Q4ZG35_HUMAN (SEQ ID NO: 177), NP_{—}003457 (SEQ ID NO: 182) or NP_{—}039246 (SEQ ID NO: 173)):

A. An isolated chimeric polypeptide encoding for HUMPAX8A_P36 (SEQ ID NO: 184), comprising a first amino acid sequence being at least about 90% homologous to MPHNSIRSGHGGLNQLGGAFVNGRPLPEVVRQRIVDLAHQGVRPCDISRQLRVSHGCVSKILGRYYETGSI RPGVIGGSKPKVATPKVVEKIGDYKRQNPTMFAWEIRDRLLAEGVCDNDTVPSVSSDIRIIRTKVQQPFNLP MDSCVATKSLSPGHTLIPSSAVTPPESPQSDSLGSTYSINGLLGIAQPGSDKRKMDDSDQDSCRLSIDSQSSS SGPRKHLRTDAFSQHHLEPLECPFERQHYPEAYASPSHTKGEQ corresponding to amino acids 1-259 of PAX8_HUMAN (SEQ ID NO:422), which also corresponds to amino acids 1-259 of HUMPAX8A_P36 (SEQ ID NO: 184), and a second amino acid sequence being at least about 70%, optionally at least 80%, preferably at least 85%, more preferably at least 90% and most preferably at least 95% homologous to a polypeptide having the sequence VRSWALGGEQGGQGPEKVYSPNEPVLHQSSWKLHQWAQGTCLMQLQA (SEQ ID NO: 507) corresponding to amino acids 260-306 of HUMPAX8A_P36 (SEQ ID NO: 184), wherein said first amino acid sequence and second amino acid sequence are contiguous and in a sequential order.

B. An isolated polypeptide encoding for an edge portion of HUMPAX8A_P36 (SEQ ID NO: 184), comprising an amino acid sequence being at least about 70%, optionally at least about 80%, preferably at least about 85%, more preferably at least about 90% and most preferably at least about 95% homologous to the sequence

(SEQ ID NO: 507)

VRSWALGGEQGGQGPEKVYSPNEPVLHQSSWKLHQWAQGTCLMQLQA
of

(SEQ ID NO: 184)

HUMPAX8A_P36.

The localization of the variant protein was determined according to results from a number of different software programs and analyses, including analyses from SignalP and other specialized programs. The variant protein is believed to be located intracellularly with regard to the cell.

Variant protein HUMPAX8A_P36 (SEQ ID NO: 184) is encoded by the following transcript(s): HUMPAX8A_T21 (SEQ ID NO: 421), for coding portion starts at position 168 and ends at position 1085. The transcript also has the following SNPs as listed in Table 101 (given according to their position on the nucleotide sequence, with the alternative nucleic acid listed.

TABLE 101
Nucleic acid SNPs

| | SNP position(s) on nucleotide |
|---|---|
| Polymorphism | sequence |
| G -> | 42 |
| A -> G | 4404 |

Variant protein HUMPAX8A_P37 (SEQ ID NO: 185) according to the present invention has an amino acid sequence as given in the “Sequence Listing” of the application; it is encoded by transcript(s) HUMPAX8A_T24 (SEQ ID NO: 156). An alignment is given to the known protein (Paired box protein Pax-8 (SEQ ID NO: 172) SEQ ID NOs:172-182) in the alignment table, on the attached CDROM. One or more alignments to one or more previously published protein sequences are given in the alignment table, on the attached CDROM. A brief description of the relationship of the variant protein according to the present invention to each such aligned protein is as follows:

1. Comparison Report Between HUMPAX8A_P37 (SEQ ID NO: 185) and PAX8_HUMAN (SEQ ID NO:422) (or Q06710-5 (SEQ ID NO: 178), NP_{—}054698 (SEQ ID NO: 181), NP_{—}039247 (SEQ ID NO: 180), Q06710-4 (SEQ ID NO: 176), NP_{—}039245 (SEQ ID NO: 179), Q06710-2 (SEQ ID NO: 175), Q4ZG35_HUMAN (SEQ ID NO: 177), NP_{—}003457 (SEQ ID NO: 182) or NP_{—}039246 (SEQ ID NO: 173)):

A. An isolated chimeric polypeptide encoding for HUMPAX8A_P37 (SEQ ID NO: 185), comprising a first amino acid sequence being at least about 90% homologous to MPHNSIRSGHGGLNQLGGAFVNGRPLPEVVRQRIVDLAHQGVRPCDISRQLRVSHGCVSKILG corresponding to amino acids 1-63 of PAX8_HUMAN (SEQ ID NO:422), which also corresponds to amino acids 1-63 of HUMPAX8A_P37 (SEQ ID NO: 185), a bridging amino acid S corresponding to amino acids 64 4 of HUMPAX8A_P37 (SEQ ID NO: 185), a second amino acid sequence being at least 90% homologous to RYYETGSIRPGVIGGSKPKVATPKVVEKIGDYKRQNPTMFAWEIRDRLLAEGVCDNDTVPSVSSINRIIRTK VQQPFNLPMDSCVATKSLSPGHTLIPSSAVTPPESPQSDSLGSTYSINGLLGIAQPGSDKRKMDDSDQDSCR LSIDSQSSSSGPRKHLRTDAFSQHHLEPLECPFERQHYPEAYASPSHTKGEQ corresponding to amino acids 64 5-259 of PAX8_HUMAN (SEQ ID NO:422), which also corresponds to amino acids 65-260 of HUMPAX8A_P37 (SEQ ID NO: 185), and a third amino acid sequence being at least about 70%, optionally at least 80%, preferably at least 85%, more preferably at least 90% and most preferably at least 95% homologous to a polypeptide having the sequence VRSWALGGEQGGQGPEKVYSPNEPVLHQSSWKLHQWAQGTCLMQLQA (SEQ ID NO: 507) corresponding to amino acids 261-307 of HUMPAX8A_P37 (SEQ ID NO: 185), wherein said first amino acid sequence, bridging amino acid, second amino acid sequence, and third amino acid sequence are contiguous and in a sequential order.

The localization of the variant protein was determined according to results from a number of different software programs and analyses, including analyses from SignalP and other specialized programs. The variant protein is believed to be located intracellularly with regard to the cell.

Variant protein HUMPAX8A_P37 (SEQ ID NO: 185) is encoded by the following transcript(s): HUMPAX8A_T24 (SEQ ID NO: 156), for which the coding portion starts at position 168 and ends at position 1088. The transcript also has the following SNPs as listed in Table 102 (given according to their position on the nucleotide sequence, with the alternative nucleic acid listed).

TABLE 102
Nucleic acid SNPs

| | SNP position(s) on nucleotide |
|---|---|
| Polymorphism | sequence |
| G -> | 42 |
| A -> G | 4407 |

Variant protein HUMPAX8A_P40 (SEQ ID NO: 186) according to the present invention has an amino acid sequence as given in the “Sequence Listing” of the application; it is encoded by transcript(s) HUMPAX8A_T30 (SEQ ID NO: 158).

The localization of the variant protein was determined according to results from a number of different software programs and analyses, including analyses from SignalP and other specialized programs. The variant protein is believed to be located intracellularly with regard to the cell.

Variant protein HUMPAX8A_P40 (SEQ ID NO: 186) is encoded by the following transcript(s): HUMPAX8A_T30 (SEQ ID NO: 158), for which the coding portion starts at position 168 and ends at position 359. The transcript also has the following SNPs as listed in Table 103 (given according to their position on the nucleotide sequence, with the alternative nucleic acid listed).

TABLE 103
Nucleic acid SNPs

| | SNP position(s) on nucleotide |
|---|---|
| Polymorphism | sequence |
| G -> | 42 |
| T -> C | 452 |
| G -> A | 2244 |

As noted above, cluster HUMPAX8A features 13 segment(s), which were listed in Table 95 above and for which the sequence(s) are given in the “Sequence Listing” of the application. These segment(s) are portions of nucleic acid sequence(s) which are described herein separately because they are of particular interest. A description of some of these segments according to the present invention is now provided.

Segment cluster HUMPAX8A_N17 (SEQ ID NO: 163) according to the present invention is supported by 3 libraries. The number of libraries was determined as previously described. This segment can be found in the following transcript(s): HUMPAX8A_T26 (SEQ ID NO: 157). Table 104 below describes the starting and ending position of this segment on each transcript.

TABLE 104
Segment location on transcripts

| | | Segment |
|---|---|---|
| | Segment | ending |
| Transcript name | starting position | position |
| HUMPAX8A_T26 (SEQ ID NO: 157) | 769 | 1049 |

Segment cluster HUMPAX8A_N19 (SEQ ID NO: 165) according to the present invention is supported by 35 libraries. The number of libraries was determined as previously described. This segment can be found in the following transcript(s): HUMPAX8A_T21 (SEQ ID NO: 421), HUMPAX8A_T24 (SEQ ID NO: 156) and HUMPAX8A_T26 (SEQ ID NO: 157). Table 105 below describes the starting and ending position of this segment on each transcript.

TABLE 105
Segment location on transcripts

| | | Segment |
|---|---|---|
| | Segment | ending |
| Transcript name | starting position | position |
| HUMPAX8A_T21 (SEQ ID NO: 421) | 945 | 2053 |
| HUMPAX8A_T24 (SEQ ID NO: 156) | 948 | 2056 |
| HUMPAX8A_T26 (SEQ ID NO: 157) | 1226 | 2334 |

Segment cluster HUMPAX8A_N21 (SEQ ID NO: 166) according to the present invention is supported by 46 libraries. The number of libraries was determined as previously described. This segment can be found in the following transcript(s): HUMPAX8A_T21 (SEQ ID NO: 421), HUMPAX8A_T24 (SEQ ID NO: 156) and HUMPAX8A_T26 (SEQ ID NO: 157). Table 106 below describes the starting and ending position of this segment on each transcript.

TABLE 106
Segment location on transcripts

| | | Segment |
|---|---|---|
| | Segment | ending |
| Transcript name | starting position | position |
| HUMPAX8A_T21 (SEQ ID NO: 421) | 2100 | 4961 |
| HUMPAX8A_T24 (SEQ ID NO: 156) | 2103 | 4964 |
| HUMPAX8A_T26 (SEQ ID NO: 157) | 2381 | 5242 |

According to an optional embodiment of the present invention, short segments related to the above cluster are also provided. These segments are up to about 120 bp in length, and so are included in a separate description.

Segment cluster HUMPAX8A_N8 (SEQ ID NO: 169) according to the present invention is supported by 6 libraries. The number of libraries was determined as previously described. This segment can be found in the following transcript(s): HUMPAX8A_T24 (SEQ ID NO: 156). Table 107 below describes the starting and ending position of this segment on each transcript.

TABLE 107
Segment location on transcripts

| | | Segment |
|---|---|---|
| | Segment | ending |
| Transcript name | starting position | position |
| HUMPAX8A_T24 (SEQ ID NO: 156) | 359 | 361 |

Segment cluster HUMPAX8A_N20 (SEQ ID NO: 171) according to the present invention is support by 12 libraries. The number of libraries was determined as previously described. This segment can be found in the following transcript(s): HUMPAX8A_T24 (SEQ ID NO: 156) and HUMPAX8A_T26 (SEQ ID NO: 157). Table 108 below describes the starting and ending position of this segment on each transcript.

TABLE 108
Segment location on transcripts

| | | Segment |
|---|---|---|
| | Segment | ending |
| Transcript name | starting position | position |
| HUMPAX8A_T24 (SEQ ID NO: 156) | 2057 | 2102 |
| HUMPAX8A_T26 (SEQ ID NO: 157) | 2335 | 2380 |

Expression of Homo sapiens paired box gene 8 (PAX8) transcripts detectable by or according to seg23-HUMPAX8A seg23 (SEQ ID NO: 397) amplicon and primers HUMAX8A seg23F (SEQ ID NO: 395) and HUMPAX8A seg23R (SEQ ID NO: 396) was measured by real time PCR. The corresponding sequences of the primers and the amplicons are given below:

Forward primer HUMPAX8A seg23F (SEQ ID NO: 395):
CTGCAGCTCTGGCAGAACC
Reverse primer HUMPAX8A seg23R (SEQ ID NO: 396):
CCCACCCTGAAGACACTCCTC
Amplicon HUMPAX8A seg23 (SEQ ID NO: 397):
CTGCAGCTCTGGCAGAACCACTAAGCTCTCAGCTCCTAGTCATCACATCT
TTTTCCGTTAACCCTGCCAAGTCAACCAGATGAGGAGTGTCTTCAGGGTG
GG

The below table 109 gives a conversion between the terms for segments (nodes) in the experimental results and those listed earlier in the description for cluster HUMPAX8A:

TABLE 109

| Segment name for HUMPAX8A | Segment name within the description |
|---|---|
| in experimental results | for cluster HUMPAX8A report |
| seg 23 (SEQ ID NO: 397) | seg 19 |

Expression of Homo sapiens Paired Box Gene 8 (PAX8)HUMPAX8A Transcripts which are Detectable by Amplicon as Depicted in Sequence Name HUMPAX8A seg23 (SEQ ID NO: 397) in Normal and Cancerous Ovary Tissues

Expression of Homo sapiens paired box gene 8 (PAX8) transcripts detectable by or according to seg23-HUMPAX8A seg23 (SEQ ID NO: 397) amplicon and primers HUMPAX8A seg23F (SEQ ID NO: 395) and HUMPAX8A seg23R (SEQ ID NO: 396) was measured by real time PCR. In parallel the expression of four housekeeping genes—PBGD (GenBank Accession No. BC019323 (SEQ ID NO:336); amplicon—PBGD-amplicon (SEQ ID NO: 302)), HPRT1 (GenBank Accession No. NM_{—}000194 (SEQ ID NO:337); amplicon—HPRT1-amplicon (SEQ ID NO: 305) (SEQ ID NO: 305)), SDHA (GenBank Accession No. NM_{—}004168 (SEQ ID NO:335); amplicon—SDHA-amplicon (SEQ ID NO:299)), and GAPDH (GenBank Accession No. BC026907 (SEQ ID NO:338) (GAPDH amplicon (SEQ ID NO: 308)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 45, 46, 48, 71, Table 1_{—}1, above: “Tissue samples in ovarian cancer testing panel”), to obtain a value of fold up-regulation for each sample relative to the median of the normal PM samples.

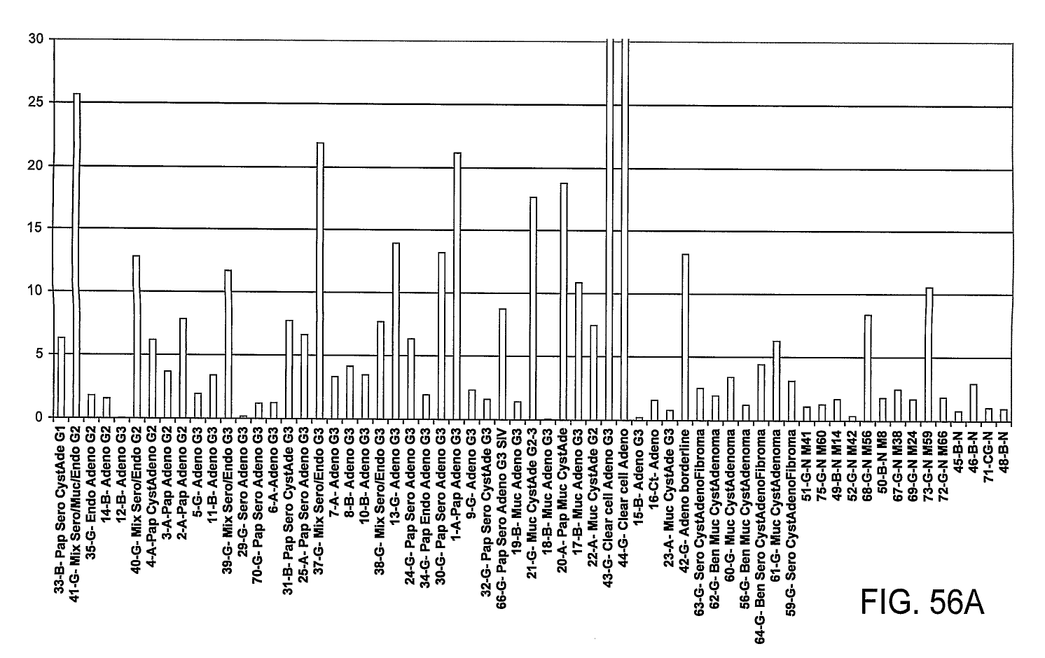

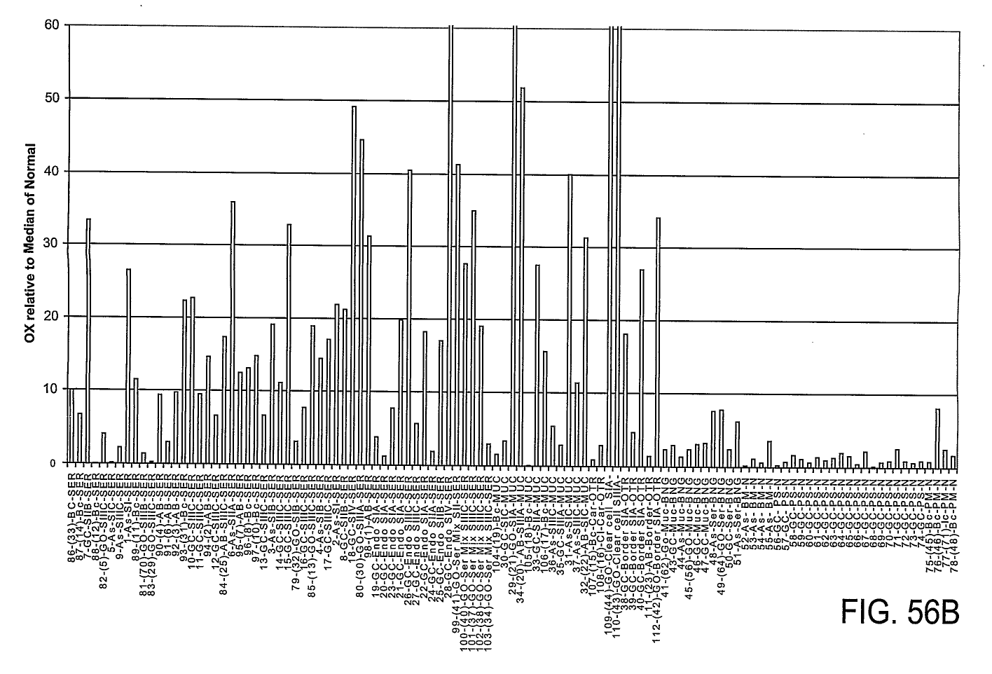

FIG. 56A is a histogram showing over expression of the Homo sapiens paired box gene 8 (PAX8) transcripts in cancerous ovary samples relative to the normal samples.

As is evident from FIG. 56A, the expression of Homo sapiens paired box gene 8 (PAX8) transcripts detectable by the above amplicon in cancer samples was significantly higher than in the non-cancerous samples (Sample Nos. 45, 46, 48, 71, Table 1_{—}1, “Tissue samples in ovarian cancer testing panel”). Notably an over-expression of at least 5 fold was found in 21 out of 41 adenocarcinoma samples.

Statistical analysis was applied to verify the significance of these results, as described below.

The P value for the difference in the expression levels of Homo sapiens paired box gene 8 (PAX8) transcripts detectable by the above amplicon in ovary cancer samples versus the normal tissue samples was determined by T test as 5.78E-03.

Primer pairs are also optionally and preferably encompassed within the present invention; for example, for the above experiment, the following primer pair was used as a non-limiting illustrative example only of a suitable primer pair: HUMPAX8A seg23F (SEQ ID NO: 395) forward primer; and HUMPAX8A seg23R (SEQ ID NO: 396) reverse primer.

The present invention also preferably encompasses any amplicon obtained through the use of any suitable primer pair; for example, for the above experiment, the following amplicon was obtained as a non-limiting illustrative example only of a suitable amplicon: HUMPAX8A seg23 (SEQ ID NO: 397).

Expression of Homo sapiens paired box gene 8 (PAX8) HUMPAX8A transcripts detectable by or according to seg23—HUMPAX8A_seg23 (SEQ ID NO: 251) amplicon and primers HUMPAX8A_seg23F (SEQ ID NO: 249) and HUMPAX8A seg23R (SEQ ID NO: 250) was further measured by real time PCR using enlarged panel as shown in Table 1_{—}5. In parallel the expression of three housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)) and G6PD (GenBank Accession No. NM_{—}000402 (SEQ ID NO:339); G6PD amplicon (SEQ ID NO: 311)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above), to obtain a value of fold up-regulation for each sample relative to the median of the normal samples.

FIG. 56B is a histogram showing over expression of the above-indicated Homo sapiens paired box gene 8 (PAX8) HUMPAX8A transcripts in cancerous Ovary samples relative to the normal samples.

As is evident from FIG. 56B, the expression of Homo sapiens paired box gene 8 (PAX8) HUMPAX8A transcripts detectable by the above amplicon in adenocarcinoma samples was significantly higher than in the non-cancerous samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above). Over expression was most significant in serous samples from patient with age above 51. Notably an over-expression of at least 8 fold was found in 47 out of 74 adenocarcinoma samples, specifically in 26 out of 38 serous carcinoma samples, in 21 out of 25 serous samples from patients with age above 51, in 5 out of 10 endometroid samples, and in 7 out of 12 mucinous carcinoma samples and.

Statistical analysis was applied to verify the significance of these results, as described below.

The P value for the difference in the expression levels of Homo sapiens paired box gene 8 (PAX8) HUMPAX8A transcripts detectable by the above amplicon in Ovary adenocarcinoma samples versus the normal tissue samples was determined by T test as 4.68e-08. The P value for the difference in the expression levels of Homo sapiens paired box gene 8 (PAX8) HUMPAX8A transcripts detectable by the above amplicon in Ovary serous carcinoma samples versus the normal tissue samples was determined by T test as 1.63e-08. The P value for the difference in the expression levels of Homo sapiens paired box gene 8 (PAX8) HUMPAX8A transcripts detectable by the above amplicon in Ovary mucinous carcinoma samples versus the normal tissue samples was determined by T test as 9.69e-003. The P value for the difference in the expression levels of Homo sapiens paired box gene 8 (PAX8) HUMPAX8A transcripts detectable by the above amplicon in Ovary endometroid samples versus the normal tissue samples was determined by T test as 9.29e-008.

Threshold of 8 fold over expression was found to differentiate between adenocarcinoma and normal samples with P value 1.82e-08 as checked by exact Fisher test. Threshold of 8 fold over expression was found to differentiate between serous carcinoma and normal samples with P value of 5.13e-08 as checked by exact Fisher test. Threshold of 8 fold over expression was found to differentiate between mucinous carcinoma and normal samples with P value of 3.54e-004 as checked by exact Fisher test. Threshold of 8 fold over expression was found to differentiate between endometroid and normal samples with P value of 1.82e-008 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: HUMPAX8A_seg23F (SEQ ID NO: 249) forward primer; and HUMPAX8A_seg23R (SEQ ID NO: 250) reverse primer.

The present invention also preferably encompasses any amplicon obtained through the use of any suitable primer pair; for example, for the above experiment, the following amplicon was obtained as a non-limiting illustrative example only of a suitable amplicon: HUMPAX8A_seg23 (SEQ ID NO: 251).

Expression of Homo sapiens Paired Box Gene 8 (PAX8) HUMPAX8A HUMPAX8A Transcripts which are Detectable by Amplicon as Depicted in Sequence Name HUMAX8A_seg23 (SEQ ID NO: 251) in Different Normal Tissues

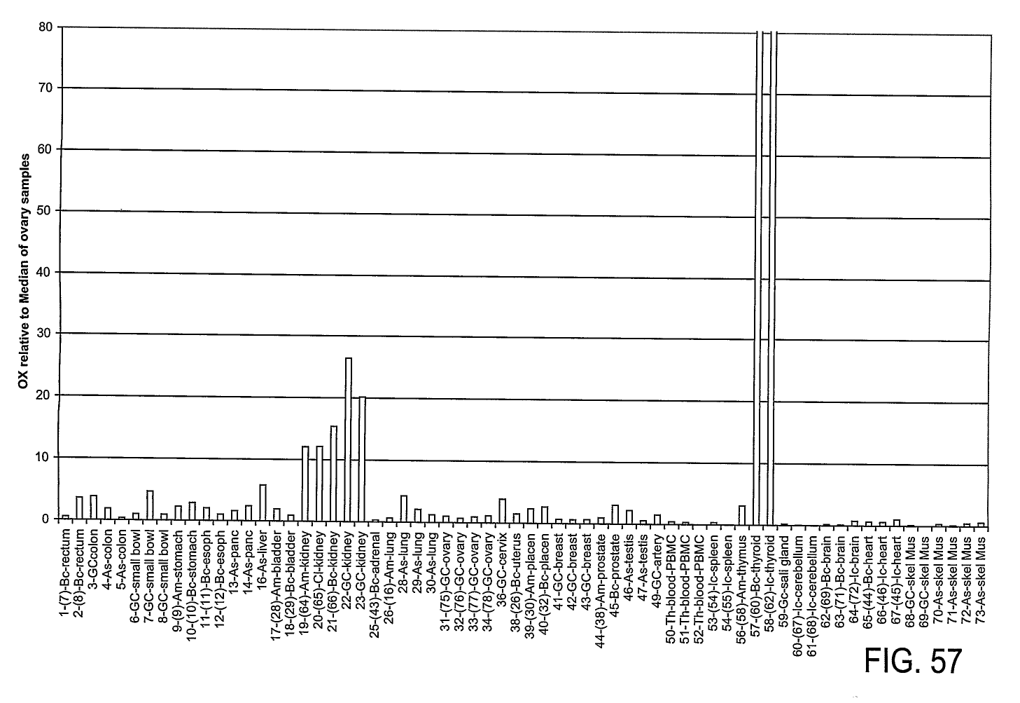

Expression of Homo sapiens paired box gene 8 (PAX8) HUMPAX8A transcripts detectable by or according to seg23_HUMPAX8A_seg23 (SEQ ID NO: 251) amplicon and primers HUMPAX8A_seg23F (SEQ ID NO: 249) and HUMPAX8A seg23R (SEQ ID NO: 250) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the ovary samples (sample numbers 31, 32, 33 and 34, Table 1_{—}10 above), to obtain a value of relative expression of each sample relative to the median of the ovary samples. FIG. 57 shows expression of Homo sapiens paired box gene 8 (PAX8) HUMPAX8A HUMPAX8A transcripts which are detectable by amplicon as depicted in sequence name HUMPAX8A_seg23 (SEQ ID NO: 251) in different normal tissues.

Expression of Homo sapiens Paired Box Gene 8 (PAX8) HUMPAX8A HUMPAX8A Transcripts which are Detectable by Amplicon as Depicted in Sequence Name HUMPAX8A seg23 (SEQ ID NO: 251) in Normal and Cancerous Breast Tissues

Expression of Homo sapiens paired box gene 8 (PAX8) HUMPAX8A HUMPAX8A transcripts detectable by or according to seg23, HUMPAX8A_seg23 (SEQ ID NO: 251) amplicon and primers HUMPAX8A_seg23F (SEQ ID NO: 249) HUMPAX8A_seg23R (SEQ ID NO: 250) was measured by real time PCR. In parallel the expression of four housekeeping genes—PBGD (GenBank Accession No. BC019323 (SEQ ID NO:336); amplicon —PBGD-amplicon (SEQ ID NO: 302)), RPL19 (GenBank Accession No. NM_{—}000981 (SEQ ID NO:342); RPL19 amplicon (SEQ ID NO: 320), SDHA (GenBank Accession No. NM_{—}004168 (SEQ ID NO:335); amplicon—SDHA-amplicon (SEQ ID NO:299)) and G6PD (GenBank Accession No. NM_{—}000402 (SEQ ID NO:339); G6PD amplicon (SEQ ID NO: 311)) was measured similarly. For each RT sample, the expression of the above amplicon was normalized to normalization factor calculated from the expression of these house keeping genes as described in normalization method 2 in the “materials and methods” section. The normalized quantity of each RT sample was then divided by the median of the quantities of the normal samples (Sample Nos. 43, 45, 46, 47, 48, 49, 50, 51, 52, 54, 56, 58, 59, 60, 61, 62, 63, 64, 65, 66, 67, 68 and 69, Table 1_{—}7, above), to obtain a value of fold differential expression for each sample relative to the median of the normal samples.

In one experiment that was carried out no differential expression in the cancerous samples relative to the normal samples was observed.

Primer pairs are also optionally and preferably encompassed within the present invention; for example, for the above experiment, the following primer pair was used as a non-limiting illustrative example only of a suitable primer pair: HUMPAX8A_seg23F (SEQ ID NO: 249) forward primer; and HUMPAX8A_seg23R (SEQ ID NO: 250) reverse primer.

The present invention also preferably encompasses any amplicon obtained through the use of any suitable primer pair; for example, for the above experiment, the following amplicon was obtained as a non-limiting illustrative example only of a suitable amplicon: HUMPAX8A_seg23 (SEQ ID NO: 251).

Expression of Homo sapiens Paired Box Gene 8 (PAX8) HUMPAX8A HUMPAX8A Transcripts which are Detectable by Amplicon as Depicted in Sequence Name HUMPAX8A_seg23 (SEQ ID NO: 251) in Normal and Cancerous Colon Tissues

Expression of Homo sapiens paired box gene 8 (PAX8) HUMPAX8A HUMPAX8A transcripts detectable by or according to seg23, HUMPAX8A_seg23 (SEQ ID NO: 251) amplicon and primers HUMPAX8A_seg23F (SEQ ID NO: 249) HUMPAX8A_seg23R (SEQ ID NO: 250) was measured by real time PCR. In parallel the expression of three housekeeping genes—PBGD (GenBank Accession No. BC019323 (SEQ ID NO:336); amplicon—PBGD-amplicon (SEQ ID NO: 302)), HPRT1 (GenBank Accession No. NM 000194 (SEQ ID NO:337); amplicon—HPRT1-amplicon (SEQ ID NO: 305) (SEQ ID NO: 305)) and G6PD (GenBank Accession No. NM_{—}000402 (SEQ ID NO:339); G6PD amplicon (SEQ ID NO: 311)) was measured similarly. For each RT sample, the expression of the above amplicon was normalized to normalization factor calculated from the expression of these house keeping genes as described in normalization method 2 in the “materials and methods” section. The normalized quantity of each RT sample was then divided by the median of the quantities of the normal samples (Sample Nos. 52, 42, 43, 44, 45, 46, 47, 48, 49, 50, 51, 53, 54, 55, 56, 57, 58, 59, 60, 61, 62, 63 and 64, Table 1_{—}8, above), to obtain a value of fold differential expression for each sample relative to the median of the normal samples.

In one experiment that was carried out no differential expression in the cancerous samples relative to the normal samples was observed.

Primer pairs are also optionally and preferably encompassed within the present invention; for example, for the above experiment, the following primer pair was used as a non-limiting illustrative example only of a suitable primer pair: HUMPAX8A_seg23F (SEQ ID NO: 249) forward primer; and HUMPAX8A_seg23R (SEQ ID NO: 250) reverse primer.

The present invention also preferably encompasses any amplicon obtained through the use of any suitable primer pair; for example, for the above experiment, the following amplicon was obtained as a non-limiting illustrative example only of a suitable amplicon: HUMAX8A_seg23 (SEQ ID NO: 251).

Expression of Homo sapiens Paired Box Gene 8 (PAX8) HUMPAX8A HUMPAX8A Transcripts which are Detectable by Amplicon as Depicted in Sequence Name HUMPAX8A_seg23 (SEQ ID NO: 251) in Normal and Cancerous Lung Tissues

Expression of Homo sapiens paired box gene 8 (PAX8) HUMPAX8A HUMPAX8A transcripts detectable by or according to seg23, HUMPAX8A_seg23 (SEQ ID NO: 251) amplicon and primers HUMPAX8A seg23F (SEQ ID NO: 249) HUMPAX8A_seg23R (SEQ ID NO: 250) was measured by real time PCR. In parallel the expression of four housekeeping genes—PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon —SDHA-amplicon (SEQ ID NO:299)), Ubiquitin (GenBank Accession No. BC000449 (SEQ ID NO:341); amplicon—Ubiquitin-amplicon (SEQ ID NO: 317)) and HPRT1 (GenBank Accession No. NM_{—}000194 (SEQ ID NO:337); amplicon—HPRT1-amplicon (SEQ ID NO: 305) (SEQ ID NO: 305)) was measured similarly. For each RT sample, the expression of the above amplicon was normalized to normalization factor calculated from the expression of these house keeping genes as described in normalization method 2 in the “materials and methods” section. The normalized quantity of each RT sample was then divided by the median of the quantities of the normal samples (Sample Nos. 51, 52, 53, 54, 55, 56, 57, 58, 59, 60, 61, 62, 63, 64, 69 and 70, Table 1_{—}6, above), to obtain a value of fold differential expression for each sample relative to the median of the normal samples.

In one experiment that was carried out no differential expression in the cancerous samples relative to the normal samples was observed.

Primer pairs are also optionally and preferably encompassed within the present invention; for example, for the above experiment, the following primer pair was used as a non-limiting illustrative example only of a suitable primer pair: HUMPAX8A_seg23F (SEQ ID NO: 249) forward primer; and HUMPAX8A_seg23R (SEQ ID NO: 250) reverse primer.

The present invention also preferably encompasses any amplicon obtained through the use of any suitable primer pair; for example, for the above experiment, the following amplicon was obtained as a non-limiting illustrative example only of a suitable amplicon: HUMPAX8A_seg23 (SEQ ID NO: 251).

### Description for Cluster N31842

Cluster N31842 (internal ID 2878310) features 3 transcript(s) and 4 segment(s) of interest, the names for which are given in Tables 110 and 111, respectively, the sequences themselves are given in the Sequence Listing. The selected protein variants are given in table 112.

TABLE 110
Transcripts of interest
Transcript Name

N31842_T4 (SEQ ID NO: 478)
N31842_T5 (SEQ ID NO: 479)
N31842_T6 (SEQ ID NO: 480)

TABLE 111
Segments of interest

Segment Name
N31842_N9 (SEQ ID NO: 481)
N31842_N11 (SEQ ID NO: 482)
N31842_N6 (SEQ ID NO: 483)
N31842_N8 (SEQ ID NO: 484)

TABLE 112
Proteins of interest

| Protein Name | Corresponding Transcript(s) |
|---|---|
| N31842_P3 (SEQ ID NO: 488) | N31842_T4 (SEQ ID NO: 478) |
| N31842_P4 (SEQ ID NO: 489) | N31842_T5 (SEQ ID NO: 479) |
| N31842_P5 (SEQ ID NO: 490) | N31842_T6 (SEQ ID NO: 480) |

These sequences are variants of the known protein Homeobox protein Hox-C10 (SEQ ID NO:485) (SwissProt accession identifier HXCA_HUMAN), referred to herein as the previously known protein.

Protein Homeobox protein Hox-C10 (SEQ ID NO:485) is known or believed to have the following function(s): Sequence-specific transcription factor which is part of a developmental regulatory system that provides cells with specific positional identities on the anterior-posterior axis. The sequence for protein Homeobox protein Hox-C10 (SEQ ID NO:485) is given. Known polymorphisms for this sequence are as shown in Table 113.

TABLE 113
Amino acid mutations for Known Protein

| SNP position(s) on amino | |
|---|---|
| acid sequence | Comment |
| 265 | A -> G |
| 271 | Missing |
| 118 | K -> N |

Protein Homeobox protein Hox-C10 (SEQ ID NO:485) localization is believed to be Nuclear.

The following GO Annotation(s) apply to the previously known protein. The following annotation(s) were found: positive regulation of cell proliferation, which are annotation(s) related to Biological Process; and RNA polymerase II transcription factor activity, which are annotation(s) related to Molecular Function.

PCT/IB2005/004037 and US Patent Application No. US 2006-0046257, filed on Jan. 27, 2005 and assigned to the applicant of the present invention, disclose the sequences of N31842_T4 (SEQ ID NO:478), N31842_T5 (SEQ ID NO:479), N31842_T6 (SEQ ID NO:480), N31842_P4 (SEQ ID NO:489) and N31842_P5 (SEQ ID NO:490) of N31842 contig, and assays and methods of use thereof in the diagnosis of lung cancer.

Unexpectedly, the sequences of N31842_T4 (SEQ ID NO:478), N31842_T5 (SEQ ID NO:479), N31842_T6 (SEQ ID NO:480), N31842_P4 (SEQ ID NO:489) and N31842_P5 (SEQ ID NO:490) of N31842 contig are now disclosed to be differentially expressed in breast cancer, ovarian cancer and renal disorders, and therefore may be contemplated within the scope of the present invention as diagnostic markers for these diseases.

Surprisingly, an N31842 variant, designated N31842_P3 (SEQ ID NO:488), is now disclosed to be differentially expressed in breast cancer, ovarian cancer, renal cancer and lung disorders, and therefore may be contemplated within the scope of the present invention as diagnostic markers for these diseases.

Surprisingly, N31842 variant polynucleotides and their respective encoded polypeptides demonstrate an age related differential diagnostic capability. Therefore, the present invention further provides diagnostic methods, kits and assays for diagnosis, assessment and prognostic indications regarding to breast cancer, according to the age of the subject, as described herein.

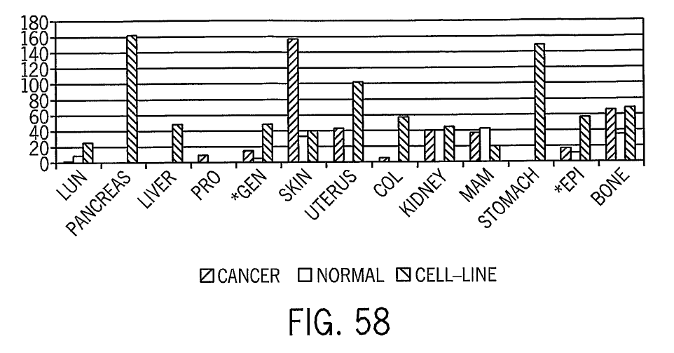

Cluster N31842 can be used as a diagnostic marker according to overexpression of transcripts of this cluster in cancer. Expression of such transcripts in normal tissues is also given according to the previously described methods. The term “number” in the left hand column of the table and the numbers on the y-axis of the FIG. 58 refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 58 and Table 114. This cluster is overexpressed (at least at a minimum level) in the following pathological conditions: a mixture of malignant tumors from different tissues and epithelial malignant tumors.

TABLE 114
Normal tissue distribution

| Name of Tissue | Number |
|---|---|
| lung | 10 |
| pancreas | 0 |
| liver | 0 |
| prostate | 0 |
| general | 6 |
| skin | 34 |
| uterus | 0 |
| colon | 0 |
| kidney | 41 |
| breast | 43 |
| stomach | 0 |
| epithelial | 11 |
| bone | 37 |

TABLE 115
P values and ratios for expression in cancerous tissue

| Name of Tissue | P1 | P2 | SP1 | R3 | SP2 | R4 |
|---|---|---|---|---|---|---|
| lung | 7.2e−01 | 3.4e−01 | 1.0e+00 | 0.6 | 6.7e−01 | 1.3 |
| pancreas | N/A | 4.2e−01 | N/A | N/A | 2.1e−02 | 2.8 |
| liver | N/A | 4.7e−01 | N/A | N/A | 4.8e−01 | 1.9 |
| prostate | 7.1e−01 | 7.6e−01 | 6.7e−01 | 1.5 | 7.5e−01 | 1.3 |
| general | 8.3e−03 | 1.1e−06 | 2.3e−02 | 2.3 | 1.8e−10 | 4.3 |
| skin | 6.7e−01 | 4.8e−01 | 1.0e−01 | 3.5 | 3.2e−01 | 1.0 |
| uterus | 6.9e−02 | 3.5e−02 | 1.9e−01 | 3.0 | 5.4e−02 | 3.1 |
| colon | 3.5e−01 | 2.6e−01 | N/A | N/A | 5.9e−01 | 1.7 |
| kidney | 4.9e−01 | 5.2e−01 | 6.1e−01 | 1.0 | 5.2e−01 | 1.0 |
| breast | 8.2e−01 | 5.3e−01 | 7.7e−01 | 1.0 | 8.5e−01 | 0.8 |
| stomach | N/A | 4.2e−01 | N/A | N/A | 6.5e−02 | 2.0 |
| epithelial | 9.1e−02 | 2.4e−03 | 1.9e−01 | 1.5 | 2.1e−04 | 2.7 |
| bone | 3.7e−01 | 4.3e−01 | 6.4e−01 | 1.4 | 4.1e−01 | 1.4 |

As noted above, cluster N31842 features 3 transcript(s), which were listed in Table 110 above. These transcript(s) encode for protein(s) which are variant(s) of protein Homeobox protein Hox-C10 (SEQ ID NO:485). A description of each variant protein according to the present invention is now provided.

Variant protein N31842_P3 (SEQ ID NO:488) according to the present invention has an amino acid sequence encoded by transcript(s) N31842_T4 (SEQ ID NO:478).

The localization of the variant protein was determined according to results from a number of different software programs and analyses, including analyses from SignalP and other specialized programs. The variant protein is believed to be secreted.

Variant protein N31842_P3 (SEQ ID NO:488) also has the following non-silent SNPs (Single Nucleotide Polymorphisms) as listed in Table 116, (given according to their position(s) on the amino acid sequence, with the alternative amino acid(s) listed).

TABLE 116
Amino acid mutations

| SNP position(s) on amino acid | |
|---|---|
| sequence | Alternative amino acid(s) |
| 37 | L -> W |

Variant protein N31842_P3 (SEQ ID NO:488) is encoded by the following transcript(s): N31842_T4 (SEQ ID NO:478), for which coding portion starts at position 1 and ends at position 456. The transcript also has the following SNPs as listed in Table 117 (given according to their position on the nucleotide sequence, with the alternative nucleic acid listed; N31842_P3 (SEQ ID NO:488)).

TABLE 117
Nucleic acid SNPs

| Polymorphism | SNP position(s) on nucleotide sequence |
|---|---|
| T -> G | 110 |
| A -> G | 537, 928 |
| C -> | 667 |
| C -> T | 1171, 1277 |
| -> T | 1459 |

Variant protein N31842_P4 (SEQ ID NO:489) according to the present invention has an amino acid sequence encoded by transcript(s) N31842_T5 (SEQ ID NO:479). An alignment is given to the known protein (Homeobox protein Hox-C10 SEQ ID NOs:485-487) in the alignment table on the attached CDROM. A brief description of the relationship of the variant protein according to the present invention to each such aligned protein is as follows:

### Comparison Report Between N31842_P4 (SEQ ID NO:489) and Known Protein(s) HXCA_HUMAN (SEQ ID NO:423) and NP_{—}059105 (SEQ ID NO: 486):

A. An isolated chimeric polypeptide encoding for N31842_P4 (SEQ ID NO:489), comprising a amino acid sequence being at least about 90% homologous to MYLTRERRLEISKTINLTDRQVKIWFQNRRMKLKKMNRENRIRELTSNFNFT corresponding to amino acids 291-342 of known protein(s) HXCA_HUMAN (SEQ ID NO:423) and NP_{—}059105 (SEQ ID NO: 486), which also corresponds to amino acids 1-52 of N31842_P4 (SEQ ID NO:489), wherein said and first amino acid sequence are contiguous and in a sequential order.

The localization of the variant protein was determined according to results from a number of different software programs and analyses, including analyses from SignalP and other specialized programs. The variant protein is believed to be located intracellularly with regard to the cell.

Variant protein N31842_P4 (SEQ ID NO:489) also has the following non-silent SNPs (Single Nucleotide Polymorphisms) as listed in Table 118, (given according to their position(s) on the amino acid sequence, with the alternative amino acid(s) listed).

TABLE 118
Amino acid mutations

| SNP position(s) on amino acid | |
|---|---|
| sequence | Alternative amino acid(s) |
| 13 | K -> R |

Variant protein N31842_P4 (SEQ ID NO:489) is encoded by the following transcript(s): N31842_T5 (SEQ ID NO:479), for which the coding portion starts at position 182 and ends at position 337. The transcript also has the following SNPs as listed in Table 119 (given according to their position on the nucleotide sequence, with the alternative nucleic acid listed).

TABLE 119
Nucleic acid SNPs

| Polymorphism | SNP position(s) on nucleotide sequence |
|---|---|
| A -> G | 219, 610 |
| C -> | 349 |
| C -> T | 853, 959 |
| -> T | 1141 |

Variant protein N31842_P5 (SEQ ID NO:490) according to the present invention has an amino acid sequence encoded by transcript N31842_T6 (SEQ ID NO:480). One or more alignments to one or more previously published protein sequences are given in the alignment table in the attached CDROM. A brief description of the relationship of the variant protein according to the present invention to each such aligned protein is as follows:

### Comparison Report Between N31842_P5 (SEQ ID NO:490) and Known Protein(s) HXCA_HUMAN (SEQ ID NO:423) and NP_{—}059105 (SEQ ID NO: 486):

A. An isolated chimeric polypeptide encoding for N31842_P5 (SEQ ID NO:490), comprising a first amino acid sequence being at least about 70%, optionally at least 80%, preferably at least 85%, more preferably at least 90% and most preferably at least 95%, homologous to a polypeptide having the sequence SQARAGCGLLSQCWCPGELTSPSS (SEQ ID NO: 533) corresponding to amino acids 1-24 of N31842_P5 (SEQ ID NO:490), and a second amino acid sequence being at least about 90% homologous to EEIKAENTTGNWLTAKSGRKKRCPYTKHQTLELEKEFLFNMYLTRERRLEISKTINLTDRQVKIWFQNRRM KLKKMNRENRIRELTSNFNFT corresponding to amino acids 251-342 of known protein(s) HXCA_HUMAN (SEQ ID NO:423) and NP_{—}059105 (SEQ ID NO: 486), which also corresponds to amino acids 25-116 of N31842_P5 (SEQ ID NO:490), wherein said first amino acid sequence and second amino acid sequence are contiguous and in a sequential order.

B. An isolated polypeptide encoding for a head of N31842_P5 (SEQ ID NO:490), comprising a polypeptide being at least 70%, optionally at least about 80%, preferably at least about 85%, more preferably at least about 90% and most preferably at least about 95% homologous to the sequence SQARAGCGLLSQCWCPGELTSPSS (SEQ ID NO: 533) of N31842_P5 (SEQ ID NO:490).

The localization of the variant protein was determined according to results from a number of different software programs and analyses, including analyses from SignalP and other specialized programs. The variant protein is believed to be located intracellularly with regard to the cell.

Variant protein N31842_P5 (SEQ ID NO:490) also has the following non-silent SNPs (Single Nucleotide Polymorphisms) as listed in Table 120, (given according to their position(s) on the amino acid sequence, with the alternative amino acid(s) listed).

TABLE 120
Amino acid mutations

| SNP position(s) on amino acid | |
|---|---|
| sequence | Alternative amino acid(s) |
| 77 | K -> R |

Variant protein N31842_P5 (SEQ ID NO:490) is encoded by the following transcript(s): N31842_T6 (SEQ ID NO:480), for which coding portion starts at position 2 and ends at position 349. The transcript also has the following SNPs as listed in Table 121 (given according to their position on the nucleotide sequence, with the alternative nucleic acid listed).

TABLE 121
Nucleic acid SNPs

| Polymorphism | SNP position(s) on nucleotide sequence |
|---|---|
| A -> G | 231, 622 |
| C -> | 361 |
| C -> T | 865, 971 |
| -> T | 1153 |

As noted above, cluster N31842 features 4 segment(s), which were listed in Table 111 above and for which the sequence(s) are given. These segment(s) are portions of nucleic acid sequence(s) which are described herein separately because they are of particular interest. A description of some of these segments according to the present invention is now provided.

Segment cluster N31842_N9 (SEQ ID NO:481) (N31842_N9 segment name in experimental results is N31842 seg 6) according to the present invention is supported by 9 libraries. The number of libraries was determined as previously described. This segment can be found in the following transcript(s): N31842_T4 (SEQ ID NO:478). Table 122 below describes the starting and ending position of this segment on each transcript.

TABLE 122
Segment location on transcripts

| | Segment | Segment |
|---|---|---|
| Transcript name | starting position | ending position |
| N31842_T4 (SEQ ID NO: 478) | 75 | 380 |

Microarray (chip) data is also available for this segment as follows. As described above with regard to the cluster itself, various oligonucleotides were tested for being differentially expressed in various disease conditions, particularly cancer. The following oligonucleotides were found to hit this segment, shown in Table 123.

TABLE 123
Oligonucleotides related to this segment

| Oligonucleotide name | Overexpressed in cancers | Chip reference |
|---|---|---|
| N31842_0_5_42 | lung malignant tumors | LUN |
| (SEQ ID NO: 493) | | |

Segment cluster N31842_N11 (SEQ ID NO:482) (N31842_N11 segment name in experimental results is N31842 seg11 (WT)) according to the present invention is supported by 66 libraries. The number of libraries was determined as previously described. This segment can be found in the following transcript(s): N31842_T4 (SEQ ID NO:478), N31842_T5 (SEQ ID NO:479) and N31842_T6 (SEQ ID NO:480). Table 124 below describes the starting and ending position of this segment on each transcript.

TABLE 124
Segment location on transcripts

| | Segment | Segment |
|---|---|---|
| Transcript name | starting position | ending position |
| N31842_T4 (SEQ ID NO: 478) | 381 | 881 |
| N31842_T5 (SEQ ID NO: 479) | 63 | 563 |
| N31842_T6 (SEQ ID NO: 480) | 75 | 575 |

Microarray (chip) data is also available for this segment as follows. As described above with regard to the cluster itself, various oligonucleotides were tested for being differentially expressed in various disease conditions, particularly cancer. The following oligonucleotides were found to hit this segment, shown in Table 125.

TABLE 125
Oligonucleotides related to this segment

| Oligonucleotide name | Overexpressed in cancers | Chip reference |
|---|---|---|
| N31842_0_13_73 | lung malignant tumors | LUN |
| N31842_0_13_73 | breast malignant tumors | BRS |

According to an optional embodiment of the present invention, short segments related to the above cluster are also provided. These segments are up to about 120 bp in length, and so are included in a separate description.

Segment cluster N31842_N6 (SEQ ID NO:483) according to the present invention is supported by 2 libraries. The number of libraries was determined as previously described. This segment can be found in the following transcript(s): N31842_T5 (SEQ ID NO:479). Table 126 below describes the starting and ending position of this segment on each transcript.

TABLE 126
Segment location on transcripts

| | Segment | Segment |
|---|---|---|
| Transcript name | starting position | ending position |
| N31842_T5 (SEQ ID NO: 479) | 1 | 62 |

Segment cluster N31842_N8 (SEQ ID NO:484) according to the present invention is supported by 1 libraries. The number of libraries was determined as previously described. This segment can be found in the following transcript(s): N31842_T4 (SEQ ID NO:478) and N31842_T6 (SEQ ID NO:480). Table 127 below describes the starting and ending position of this segment on each transcript.

TABLE 127
Segment location on transcripts

| | Segment | Segment |
|---|---|---|
| Transcript name | starting position | ending position |
| N31842_T4 (SEQ ID NO: 478) | 1 | 74 |
| N31842_T6 (SEQ ID NO: 480) | 1 | 74 |

Expression of Homo sapiens homeo box C10 (HOXC10) transcripts detectable by or according to segments as described below was measured by real time PCR: seg8—N31842 seg8WT (SEQ ID NO: 400) amplicon and primers N31842 seg8WTF (SEQ ID NO: 398) and N31842 seg8WTR (SEQ ID NO: 399); seg6-N31842_seg6 (SEQ ID NO: 403) amplicon and primers N31842_seg6F (SEQ ID NO: 401) and N31842_seg6R (SEQ ID NO: 402).

The sequences of the corresponding amplicons and primers are given below:

Forward primer N31842 seg8WTF (SEQ ID NO: 398):
AATGCAATGCGACTGCAAAA
Reverse primer N31842 seg8WTR (SEQ ID NO: 399):
CATTTCTCATGCTTTAAGTGGAAGC
Amplicon N31842 seg8WT (SEQ ID NO: 400):
AATGCAATGCGACTGCAAAAAAGGCAAAGACCTCAGACTCTCCTTCCAAG
GGACCTGTGGTTCGTGCTGCGAAGATGCTTCCACTTAAAGCATGAGAAAT
G
Forward primer N31842_seg6F (SEQ ID NO: 401):
GCGAAACGCGATTTGTTGTT
Reverse primer N31842_seg6R (SEQ ID NO: 402):
CATCTGGAGGAGGGAGGGA
Amplicon N31842_seg6 (SEQ ID NO: 403):
GCGAAACGCGATTTGTTGTTTGTGGGTCTGATTTGTGCGTGCGGCTTGGG
CTCCTGCGGCTTTTGGCTCGGCCGGGGGCCTTGGGCAGCGAGGCTGGAGC
CGGAAGAGGTGGAGGTGAAGGGCTGCCCGCCACGTCCCTCCCTCCTCCAG
ATG

The below table 128 gives a conversion between the terms for segments (nodes) in the experimental results and those listed earlier in the description for cluster N31842:

TABLE 128

| | Segment name within the |
|---|---|
| Segment name for N31842 | description for cluster N31842 |
| in experimental results | report |
| seg 6 (SEQ ID NO: 403) | seg 9 |
| Seg8WT (SEQ ID NO: 400) | Seg11 |

Expression of Homo sapiens Homeo Box C10 (HOXC10) N31842 Transcripts which are Detectable by Amplicon as Depicted in Sequence Name N31842 seg8WT (SEQ ID NO: 400) in Normal and Cancerous Lung Tissues

Expression of Homo sapiens homeo box C10 (HOXC10) transcripts detectable by or according to seg8-N31842 seg8WT (SEQ ID NO: 400) amplicon and primers N31842 seg8WTF (SEQ ID NO: 398) and N31842 seg8WTR (SEQ ID NO: 399) was measured by real time PCR. In parallel the expression of four housekeeping genes—PBGD (GenBank Accession No. BC019323 (SEQ ID NO:336); amplicon—PBGD-amplicon (SEQ ID NO: 302)), HPRT1 (GenBank Accession No. NM_{—}000194 (SEQ ID NO:337); amplicon—HPRT1-amplicon (SEQ ID NO: 305) (SEQ ID NO: 305)), Ubiquitin (GenBank Accession No. BC000449 (SEQ ID NO:341); amplicon—Ubiquitin-amplicon (SEQ ID NO: 317)) and SDHA (GenBank Accession No. NM_{—}004168 (SEQ ID NO:335); amplicon—SDHA-amplicon (SEQ ID NO:299)), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 47-50, 90-93, 96-99, Table 1_{—}2 above), to obtain a value of fold up-regulation for each sample relative to the median of the normal PM samples.

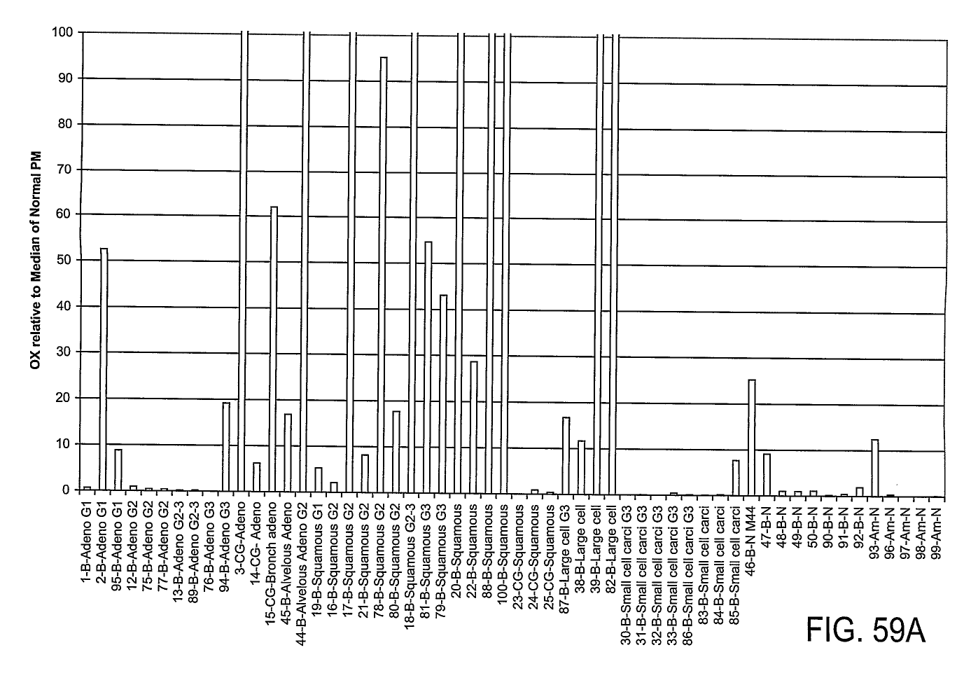

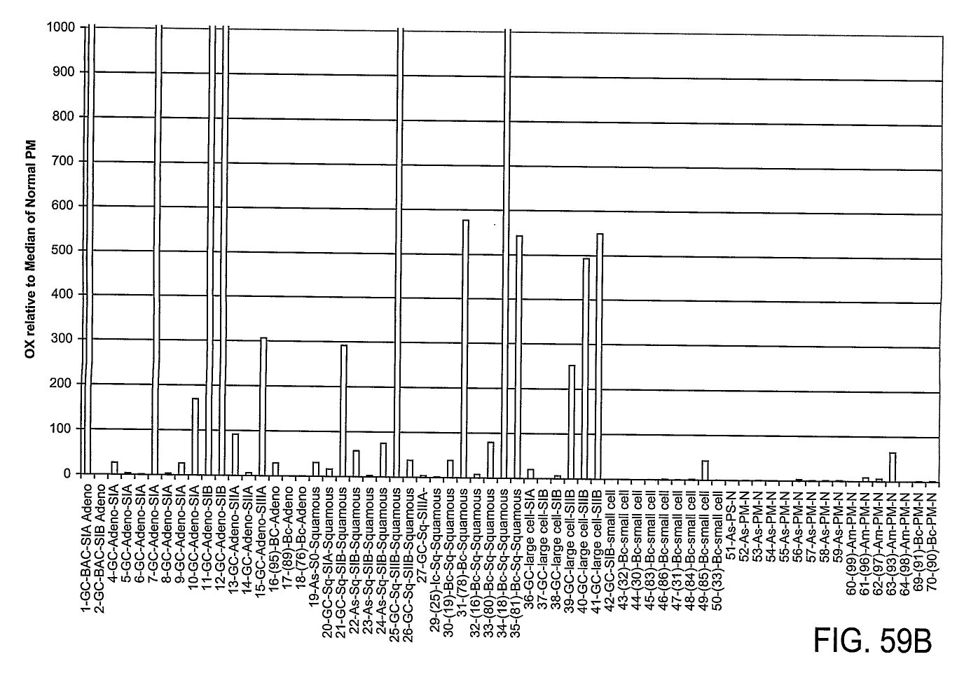

FIG. 59A is a histogram showing over expression of the Homo sapiens homeo box C10 (HOXC10) transcripts detectable by or according to N31842 seg8WT (SEQ ID NO: 400) in cancerous lung samples relative to the normal samples.

As is evident from FIG. 59A, the expression of Homo sapiens homeo box C10 (HOXC10) transcripts detectable by the above amplicon in cancer samples was significantly higher than in the non-cancerous samples (Sample Nos. 46-50, 90-93, 96-99 Table 1_{—}2). Notably an over-expression of at least 5 fold was found in 8 out of 15 adenocarcinoma samples, 12 out of 16 squamous cell carcinoma samples, 4 out of 4 large cell carcinoma samples and in 1 out of 8 small cells carcinoma samples.

Statistical analysis was applied to verify the significance of these results, as described below.

The P value for the difference in the expression levels of Homo sapiens homeo box C10 (HOXC10) transcripts detectable by the above amplicon in lung cancer samples versus the normal tissue samples was determined by T test as 1.17E-02 in Squamous cell carcinoma.

Threshold of 5 fold overexpression was found to differentiate between cancer and normal samples with P value of 4.76E-03 as checked by exact fisher test in Squamous cell carcinoma. The above values demonstrate statistical significance of the results.

Primer pairs are also optionally and preferably encompassed within the present invention; for example, for the above experiment, the following primer pair was used as a non-limiting illustrative example only of a suitable primer pair: N31842 seg8WTF (SEQ ID NO: 398) forward primer; and N31842 seg8WTR (SEQ ID NO: 399) reverse primer.

The present invention also preferably encompasses any amplicon obtained through the use of any suitable primer pair; for example, for the above experiment, the following amplicon was obtained as a non-limiting illustrative example only of a suitable amplicon: N31842 seg8WT (SEQ ID NO: 400).

Expression of Homo sapiens homeo box C10 (HOXC10) transcripts detectable by or according to seg8WT—N31842_seg8WT (SEQ ID NO: 400) amplicon and primers N31842_seg8WTF (SEQ ID NO: 398) and N31842_seg8WTR (SEQ ID NO: 399) was further measured by real time PCR using enlarged panel as shown in Table 1_{—}6. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 51, 52, 53, 54, 55, 56, 57, 58, 59, 60, 61, 62, 63, 64, 69 and 70, Table 1_{—}6 above), to obtain a value of fold up-regulation for each sample relative to the median of the normal samples.

FIG. 59B is a histogram showing over expression of the above-indicated Homo sapiens homeo box C10 (HOXC10) transcripts in cancerous Lung samples relative to the normal samples.

As is evident from FIG. 59B, the expression of Homo sapiens homeo box C10 (HOXC10) transcripts detectable by the above amplicon in non-small cell carcinoma samples was significantly higher than in the non-cancerous samples (sample numbers 51, 52, 53, 54, 55, 56, 57, 58, 59, 60, 61, 62, 63, 64, 69 and 70, Table 1_{—}6 above). Notably an over-expression of at least 10 fold was found in 26 out of 39 non-small cell carcinoma samples, specifically in 10 out of 17 adenocarcinoma samples, in 12 out of 16 squamous cell carcinoma samples, and in 4 out of 6 large cell carcinoma samples.

Statistical analysis was applied to verify the significance of these results, as described below.

The P value for the difference in the expression levels of Homo sapiens homeo box C10 (HOXC10) transcripts detectable by the above amplicon in Lung non-small cell carcinoma samples versus the normal tissue samples was determined by T test as 1.12e-002.

Threshold of 10 fold over expression was found to differentiate between non-small cell carcinoma and normal samples with P value of 3.49e-005 as checked by exact Fisher test. Threshold of 10 fold over expression was found to differentiate between adenocarcinoma and normal samples with P value of 1.66e-003 as checked by exact Fisher test. Threshold of 10 fold over expression was found to differentiate between squamous cell carcinoma and normal samples with P value of 8.50e-005 as checked by exact Fisher test. Threshold of 10 fold over expression was found to differentiate between large cell carcinoma and normal samples with P value of 9.23e-003 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: N31842_seg8WTF (SEQ ID NO: 398) forward primer; and N31842_seg8WTR (SEQ ID NO: 399) reverse primer.

The present invention also preferably encompasses any amplicon obtained through the use of any suitable primer pair; for example, for the above experiment, the following amplicon was obtained as a non-limiting illustrative example only of a suitable amplicon: N31842_seg8WT (SEQ ID NO: 400).

Expression of Homo sapiens Homeo Box C10 (HOXC10)N31842 Transcripts which are Detectable by Amplicon as Depicted in Sequence Name N31842_seg8 (SEQ ID NO: 400) in Different Normal Tissues

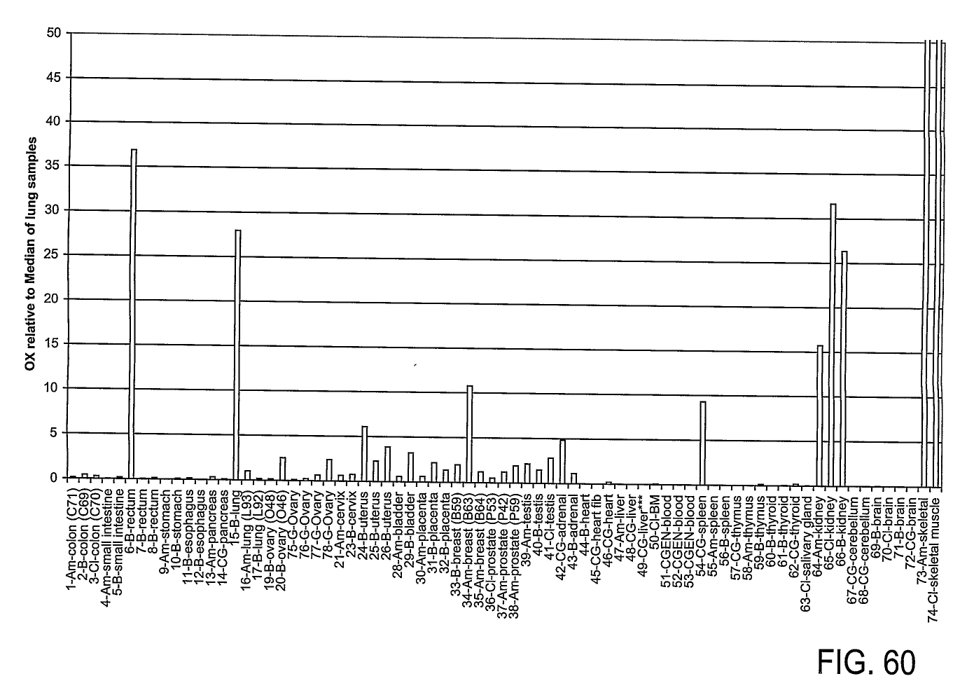

Expression of Homo sapiens homeo box C10 (HOXC10) transcripts detectable by or according to seg8-N31842_seg8 (SEQ ID NO: 400) amplicon and primers N31842_seg8F (SEQ ID NO: 398) and N31842_seg8R (SEQ ID NO: 399) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the geometric mean of the quantities of the housekeeping genes. The normalized quantity of each RT sample was then divided by the median of the quantities of the lung samples (sample numbers 15, 16 and 17, Table 1_{—}9 above), to obtain a value of relative expression of each sample relative to the median of the lung samples. FIG. 60 shows expression of Homo sapiens homeo box C10 (HOXC10)N31842 transcripts which are detectable by amplicon as depicted in sequence name N31842_seg8 (SEQ ID NO: 400) in different normal tissues.

Expression of Homo sapiens Homeo Box C10 (HOXC10)N31842 Transcripts which are Detectable by Amplicon as Depicted in Sequence Name N31842_seg8 (SEQ ID NO: 400) in Normal and Cancerous Breast Tissues

Expression of Homo sapiens homeo box C10 (HOXC10) transcripts detectable by or according to seg8-N31842_seg8 (SEQ ID NO: 400) amplicon and primers N31842_seg8F (SEQ ID NO: 398) and N31842_seg8R (SEQ ID NO: 399) was measured by real time PCR. In parallel the expression of four housekeeping genes—G6PD (GenBank Accession No. NM_{—}000402 (SEQ ID NO:339); G6PD amplicon (SEQ ID NO: 311)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 57, 59, 60, 63, 66, 64, 56, 65, 67 and 58, Table 1_{—}3 above), to obtain a value of fold up-regulation for each sample relative to the median of the normal PM samples.

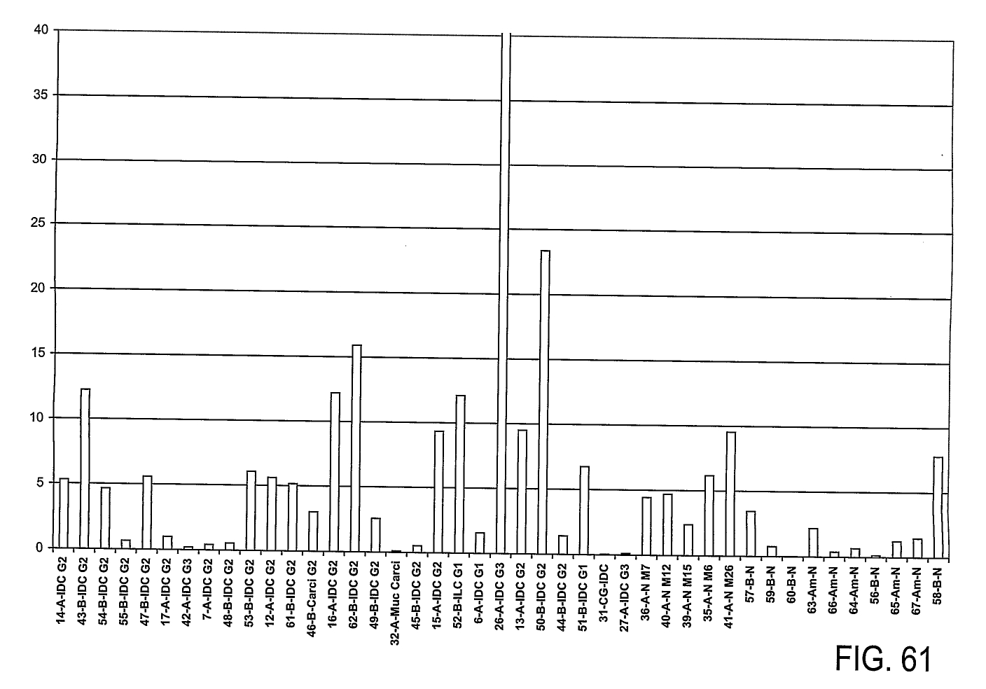

FIG. 61 is a histogram showing over expression of the Homo sapiens homeo box C10 (HOXC10) transcripts detectable by or according to N31842_seg8 (SEQ ID NO: 400) in cancerous breast samples relative to the normal samples.

As is evident from FIG. 61, the expression of Homo sapiens homeo box C10 (HOXC₁₀) transcripts detectable by the above amplicon in cancer samples was significantly higher than in the non-cancerous samples (sample numbers 57, 59, 60, 63, 66, 64, 56, 65, 67 and 58, Table 1_{—}3 above), mainly in patient with age above 50.

Notably an over-expression of at least 5 fold was found in 14 out of 28 adenocarcinoma samples.

Statistical analysis was applied to verify the significance of these results, as described below.

Threshold of 5 fold overexpression was found to differentiate between cancer and normal samples with P value of 2.83E-02 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: N31842_seg8F (SEQ ID NO: 398) forward primer; and N31842_seg8R (SEQ ID NO: 399) reverse primer.

The present invention also preferably encompasses any amplicon obtained through the use of any suitable primer pair; for example, for the above experiment, the following amplicon was obtained as a non-limiting illustrative example only of a suitable amplicon: N31842_seg8 (SEQ ID NO: 400).

Expression of Homo sapiens Homeo Box C10 (HOXC10) N31842 Transcripts which are Detectable by Amplicon as Depicted in Sequence Name N31842_seg6 (SEQ ID NO: 403) in Normal and Cancerous Breast Tissues

Expression of Homo sapiens homeo box C10 (HOXC10) transcripts detectable by or according to seg6-N31842_seg6 (SEQ ID NO: 403) amplicon and primers N31842_seg6F (SEQ ID NO: 401) and N31842_seg6R (SEQ ID NO: 402) was measured by real time PCR. In parallel the expression of four housekeeping genes—G6PD (GenBank Accession No. NM_{—}000402 (SEQ ID NO:339); G6PD amplicon (SEQ ID NO: 311)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 57, 59, 60, 63, 66, 64, 56, 65, 67 and 58, Table 1_{—}3 above), to obtain a value of fold up-regulation for each sample relative to the median of the normal PM samples.

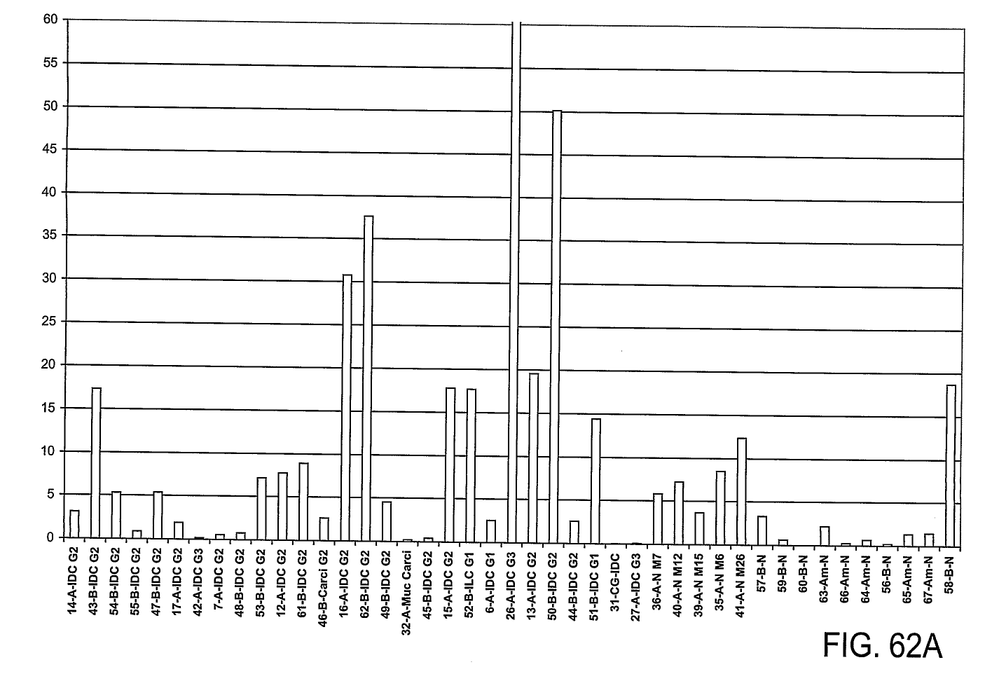

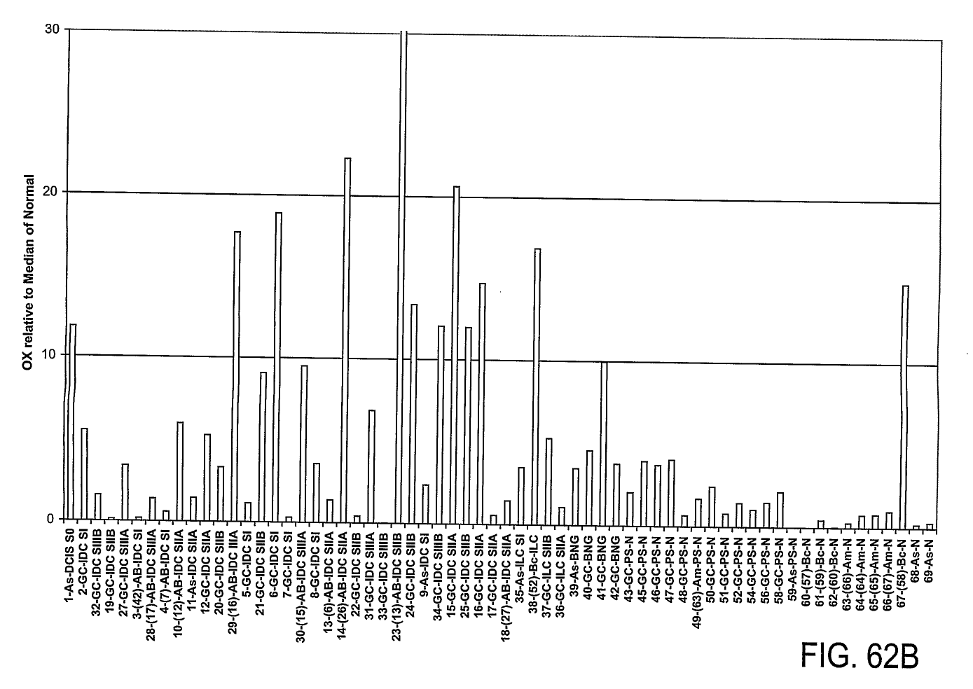

FIG. 62A is a histogram showing over expression of the Homo sapiens homeo box C10 (HOXC10) detectable by or according to N31842_seg6 (SEQ ID NO: 403) in cancerous Breast samples relative to the normal samples.

As is evident from FIG. 62A, the expression of Homo sapiens homeo box C10 (HOXC10) transcripts detectable by the above amplicon in cancer samples was significantly higher than in the non-cancerous samples (sample numbers 57, 59, 60, 63, 66, 64, 56, 65, 67 and 58, Table 1_{—}3 above), mainly in patient with age above 50. Notably an over-expression of at least 5 fold was found in 14 out of 28 adenocarcinoma samples.

Statistical analysis was applied to verify the significance of these results, as described below.

Threshold of 5 fold overexpression was found to differentiate between cancer and normal samples with P value of 2.83E-02 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: N31842_seg6F (SEQ ID NO: 401) forward primer; and N31842_seg6R (SEQ ID NO: 402) reverse primer.

The present invention also preferably encompasses any amplicon obtained through the use of any suitable primer pair; for example, for the above experiment, the following amplicon was obtained as a non-limiting illustrative example only of a suitable amplicon: N31842_seg6 (SEQ ID NO: 403).

Expression of Homo sapiens homeo box C10 (HOXC10) transcripts detectable by or according to seg6-N31842_seg6 (SEQ ID NO: 403) amplicon and primers N31842_seg6F (SEQ ID NO: 401) and N31842_seg6R (SEQ ID NO: 402) was further measured by real time PCR using enlarged panel as shown in Table 1_{—}7. In parallel the expression of four housekeeping genes—G6PD (GenBank Accession No. NM_{—}000402 (SEQ ID NO:339); G6PD amplicon (SEQ ID NO: 311)), RPL19 (GenBank Accession No. NM_{—}000981 (SEQ ID NO:342); RPL19 amplicon (SEQ ID NO: 320)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 43, 45, 46, 47, 48, 49, 50, 51, 52, 54, 56, 58, 59, 60, 61, 62, 63, 64, 65, 66, 67, 68 and 69, Table 1_{—}7 above), to obtain a value of fold up-regulation for each sample relative to the median of the normal samples.

FIG. 62B is a histogram showing over expression of the above-indicated Homo sapiens homeo box C10 (HOXC10) transcripts in cancerous Breast samples relative to the normal samples.

As is evident from FIG. 62B, the expression of Homo sapiens homeo box C10 (HOXC10) transcripts detectable by the above amplicon in cancer samples was significantly higher than in the non-cancerous samples (sample numbers 43, 45, 46, 47, 48, 49, 50, 51, 52, 54, 56, 58, 59, 60, 61, 62, 63, 64, 65, 66, 67, 68 and 69, Table 1_{—}7 above). Notably an over-expression of at least 10 fold was found in 11 out of 37 adenocarcinoma samples. The over expression was observed mainly in patients with age above 48—in 10 of 21 IDC patients with age above 48.

Statistical analysis was applied to verify the significance of these results, as described below.

The P value for the difference in the expression levels of Homo sapiens homeo box C10 (HOXC10) transcripts detectable by the above amplicon in all Breast cancer samples versus the normal tissue samples was determined by T test as 5.69e-004 for all adenocarcinoma samples.

Threshold of 10 fold over expression was found to differentiate between all cancer samples and normal samples with P value of 1.53e-002 as checked by exact Fisher test. Threshold of 10 fold over expression was found to differentiate between IDC samples from patients with age above 48 and normal samples with P value of 2.87e-03 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: N31842_seg6F (SEQ ID NO: 401) forward primer; and N31842_seg6R (SEQ ID NO: 402) reverse primer.

The present invention also preferably encompasses any amplicon obtained through the use of any suitable primer pair; for example, for the above experiment, the following amplicon was obtained as a non-limiting illustrative example only of a suitable amplicon: N31842_seg6 (SEQ ID NO: 403).

Expression of Homo sapiens Homeo Box C10 (HOXC10) N31842 Transcripts which are Detectable by Amplicon as Depicted in Sequence Name N31842_seg6 (SEQ ID NO: 403) in Normal and Cancerous Colon Tissues

Expression of Homo sapiens homeo box C10 (HOXC10) transcripts detectable by or according to seg6, N31842_seg6 (SEQ ID NO: 403) amplicon and primers N31842_seg6F (SEQ ID NO: 401) N31842_seg6R (SEQ ID NO: 402) was measured by real time PCR. In parallel the expression of four housekeeping genes—PBGD (GenBank Accession No. BC019323 (SEQ ID NO:336); amplicon—PBGD-amplicon (SEQ ID NO: 302)), HPRT1 (GenBank Accession No. NM_{—}000194 (SEQ ID NO:337); amplicon—HPRT1-amplicon (SEQ ID NO: 305) (SEQ ID NO: 305)) and G6PD (GenBank Accession No. NM_{—}000402 (SEQ ID NO:339); G6PD amplicon (SEQ ID NO: 311)), was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (Sample Nos. 52, 42, 43, 44, 45, 46, 47, 48, 49, 50, 51, 53, 54, 55, 56, 57, 58, 59, 60, 61, 62, 63 and 64, Table 1_{—}8, above), to obtain a value of fold differential expression for each sample relative to the median of the normal samples.

In one experiment that was carried out no differential expression in the cancerous samples relative to the normal samples was observed.

Primer pairs are also optionally and preferably encompassed within the present invention; for example, for the above experiment, the following primer pair was used as a non-limiting illustrative example only of a suitable primer pair: N31842_seg6F (SEQ ID NO: 401) forward primer; and N31842_seg6R (SEQ ID NO: 402) reverse primer.

The present invention also preferably encompasses any amplicon obtained through the use of any suitable primer pair; for example, for the above experiment, the following amplicon was obtained as a non-limiting illustrative example only of a suitable amplicon: N31842_seg6 (SEQ ID NO: 403).

Expression of Homo sapiens Homeo Box C10 (HOXC10) N31842 Transcripts which are Detectable by Amplicon as Depicted in Sequence Name N31842_seg6 (SEQ ID NO: 403) in Different Normal Tissues

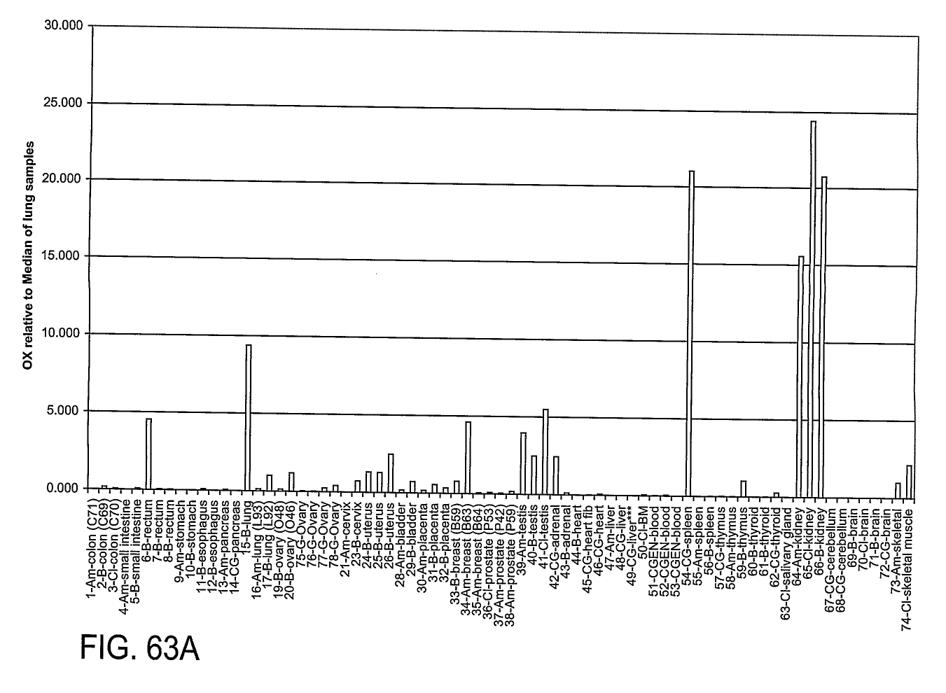

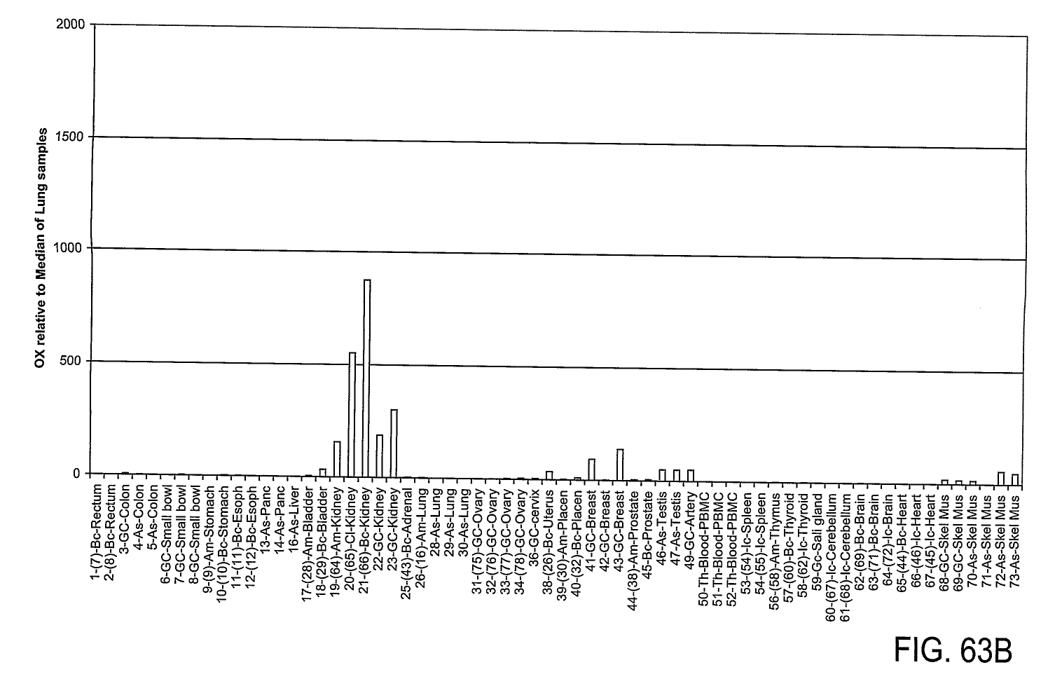

Expression of Homo sapiens homeo box C10 (HOXC10) transcripts detectable by or according to seg6-N31842_seg6 (SEQ ID NO: 403) amplicon and primers N31842_seg6F (SEQ ID NO: 401) and N31842_seg6R (SEQ ID NO: 402) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the geometric mean of the quantities of the housekeeping genes. The normalized quantity of each RT sample was then divided by the median of the quantities of the lung samples (sample numbers 15, 16 and 17, Table 1_{—}9 above), to obtain a value of relative expression of each sample relative to the median of the lung samples. FIG. 63A shows expression of Homo sapiens homeo box C10 (HOXC10)N31842 transcripts which are detectable by amplicon as depicted in sequence name N31842_seg6 (SEQ ID NO: 403) in different normal tissues.

Expression of Homo sapiens homeo box C10 (HOXC10) transcripts detectable by or according to seg6—N31842_seg6 (SEQ ID NO: 403) amplicon and primers N31842_seg6F (SEQ ID NO: 401) and N31842_seg6R (SEQ ID NO: 402) was further measured by real time PCR using updated panel as shown in Table 1_{—}10. In parallel the expression of three housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)) and TATA box (GenBank Accession No. NM_{—}003194 (SEQ ID NO:343); TATA amplicon (SEQ ID NO: 323)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the Lung samples (sample numbers 26, 27 and 29, Table 1_{—}10 above), to obtain a value of relative expression of each sample relative to the median of the Lung samples.

As shown in FIG. 63B Homo sapiens homeo box C10 (HOXC10) transcripts detectable by the above amplicon are highly expressed in kidney tissues.

Expression of Homo sapiens Homeo Box C10 (HOXC10)N31842 Transcripts which are Detectable by Amplicon as Depicted in Sequence Name N31842_seg6 (SEQ ID NO: 403) in Normal and Cancerous Ovary Tissues

Expression of Homo sapiens homeo box C10 (HOXC10) transcripts detectable by or according to seg6—N31842_seg6 (SEQ ID NO: 403) amplicon and primers N31842_seg6F (SEQ ID NO: 401) and N31842_seg6R (SEQ ID NO: 402) was measured by real time PCR. In parallel the expression of three housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)), G6PD (GenBank Accession No. NM_{—}000402 (SEQ ID NO:339); G6PD amplicon (SEQ ID NO: 311)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above), to obtain a value of fold up-regulation for each sample relative to the median of the normal samples.

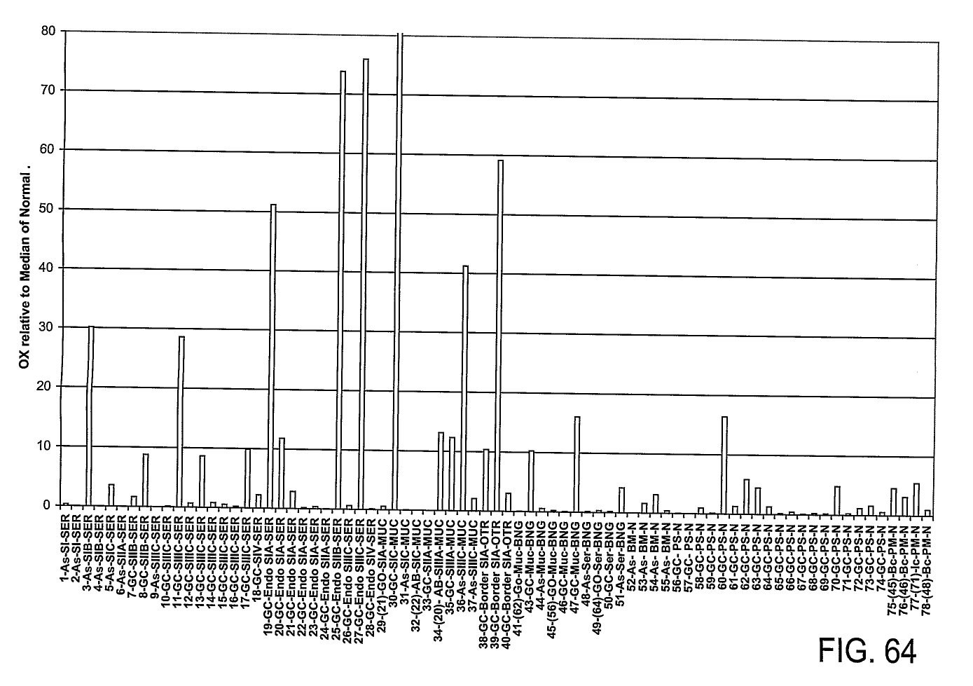

FIG. 64 is a histogram showing over expression of the above-indicated Homo sapiens homeo box C10 (HOXC10) transcripts in cancerous Ovary samples relative to the normal samples.

As is evident from FIG. 64, the expression of Homo sapiens homeo box C10 (HOXC10) transcripts detectable by the above amplicon in serous carcinoma and adenocarcinoma samples was significantly higher than in the non-cancerous samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above) and was higher in a few mucinous carcinoma samples than in the non-cancerous samples. Notably an over-expression of at least 10 fold was found in 12 out of 40 adenocarcinoma samples, specifically in 2 out of 18 serous carcinoma samples, in 4 out of 10 endometroid samples, and in 4 out of 9 mucinous carcinoma samples.

Statistical analysis was applied to verify the significance of these results, as described below.

The P value for the difference in the expression levels of Homo sapiens homeo box C10 (HOXC10) transcripts detectable by the above amplicon in Ovary adenocarcinoma samples versus the normal tissue samples was determined by T test as 7.12e-03.

Threshold of 10 fold over expression was found to differentiate between adenocarcinoma and normal samples with P value of 6.42e-03 as checked by exact Fisher test.

Primer pairs are also optionally and preferably encompassed within the present invention; for example, for the above experiment, the following primer pair was used as a non-limiting illustrative example only of a suitable primer pair: N31842_seg6F (SEQ ID NO: 401) forward primer; and N31842_seg6R (SEQ ID NO: 402) reverse primer.

The present invention also preferably encompasses any amplicon obtained through the use of any suitable primer pair; for example, for the above experiment, the following amplicon was obtained as a non-limiting illustrative example only of a suitable amplicon: N31842_seg6 (SEQ ID NO: 403).

Expression of Homo sapiens Homeo Box C10 (HOXC10) N31842 Transcripts which are Detectable by Amplicon as Depicted in Sequence Name N31842_seg6 (SEQ ID NO: 403) in Normal and Cancerous Lung Tissues

Expression of Homo sapiens homeo box C10 (HOXC10) transcripts detectable by or according to seg6—N31842_seg6 (SEQ ID NO: 403) amplicon and primers N31842_seg6F (SEQ ID NO: 401) and N31842_seg6R (SEQ ID NO: 402) was measured by real time PCR. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 51, 52, 53, 54, 55, 56, 57, 58, 59, 60, 61, 62, 63, 64, 69 and 70, Table 1_{—}6 above), to obtain a value of fold up-regulation for each sample relative to the median of the normal samples.

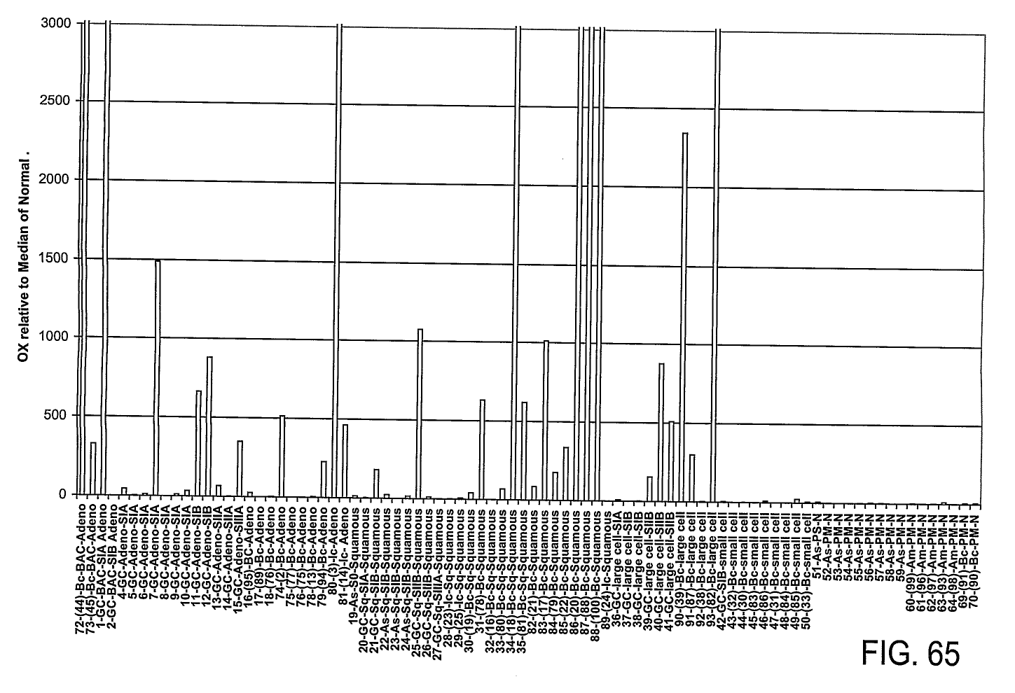

FIG. 65 is a histogram showing over expression of the above-indicated Homo sapiens homeo box C10 (HOXC10) transcripts in cancerous Lung samples relative to the normal samples.

As is evident from FIG. 65, the expression of Homo sapiens homeo box C10 (HOXC10) transcripts detectable by the above amplicon in non-small cell carcinoma samples was significantly higher than in the non-cancerous samples (sample numbers 51, 52, 53, 54, 55, 56, 57, 58, 59, 60, 61, 62, 63, 64, 69 and 70, Table 1_{—}6 above) and was higher in a few small cell carcinoma samples than in the non-cancerous samples. Notably an over-expression of at least 5 fold was found in 44 out of 61 non-small cell carcinoma samples, specifically in 18 out of 26 adenocarcinoma samples, in 19 out of 25 squamous cell carcinoma samples, in 7 out of 10 large cell carcinoma samples, and also in 3 out of 9 small cell carcinoma samples and

Statistical analysis was applied to verify the significance of these results, as described below.

The P value for the difference in the expression levels of Homo sapiens homeo box C10 (HOXC10) transcripts detectable by the above amplicon in Lung non-small cell carcinoma samples versus the normal tissue samples was determined by T test as 2.17e-004. The P value for the difference in the expression levels of Homo sapiens homeo box C10 (HOXC10) transcripts detectable by the above amplicon in Lung adenocarcinoma samples versus the normal tissue samples was determined by T test as 1.35e-002. The P value for the difference in the expression levels of Homo sapiens homeo box C10 (HOXC10) transcripts detectable by the above amplicon in Lung squamous cell carcinoma samples versus the normal tissue samples was determined by T test as 1.81 e-002.

Threshold of 5 fold over expression was found to differentiate between non-small cell carcinoma and normal samples with P value of 8.25e-004 as checked by exact Fisher test. Threshold of 5 fold over expression was found to differentiate between adenocarcinoma and normal samples with P value of 6.20e-003 as checked by exact Fisher test. Threshold of 5 fold over expression was found to differentiate between squamous cell carcinoma and normal samples with P value of 1.72e-003 as checked by exact Fisher test. Threshold of 5 fold over expression was found to differentiate between large cell carcinoma and normal samples with P value of 3.05e-002 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: N31842_seg6F (SEQ ID NO: 401) forward primer; and N31842_seg6R (SEQ ID NO: 402) reverse primer.

The present invention also preferably encompasses any amplicon obtained through the use of any suitable primer pair; for example, for the above experiment, the following amplicon was obtained as a non-limiting illustrative example only of a suitable amplicon: N31842_seg6 (SEQ ID NO: 403).

### Description for Cluster T58132

Cluster T58132 features 7 transcript(s) and 11 segment(s) of interest, the names for which are given in Tables 129 and 130, respectively, the sequences themselves are given in the “Sequence Listing” of the application. The selected protein variants are given in table 131.

TABLE 129
Transcripts of interest
Transcript Name

T58132_T2 (SEQ ID NO: 187)
T58132_T4 (SEQ ID NO: 188)
T58132_T7 (SEQ ID NO: 189)
T58132_T9 (SEQ ID NO: 190)
T58132_T10 (SEQ ID NO: 191)
T58132_T11 (SEQ ID NO: 192)
T58132_T12 (SEQ ID NO: 193)

TABLE 130
Segments of interest
Segment Name

T58132_N6 (SEQ ID NO: 194)
T58132_N8 (SEQ ID NO: 195)
T58132_N9 (SEQ ID NO: 196)
T58132_N13 (SEQ ID NO: 197)
T58132_N15 (SEQ ID NO: 198)
T58132_N21 (SEQ ID NO: 199)
T58132_N2 (SEQ ID NO: 200)
T58132_N4 (SEQ ID NO: 201)
T58132_N11 (SEQ ID NO: 202)
T58132_N17 (SEQ ID NO: 203)
T58132_N19 (SEQ ID NO: 204)

TABLE 131
Proteins of interest

| Protein Name | Corresponding Transcript(s) |
|---|---|
| T58132_P0 (SEQ ID NO: 213) | T58132_T4 (SEQ ID NO: 188) |
| T58132_P1 (SEQ ID NO: 214) | T58132_T7 (SEQ ID NO: 189) |
| T58132_P2 (SEQ ID NO: 215) | T58132_T10 (SEQ ID NO: 191); |
| | T58132_T9 (SEQ ID NO: 190) |
| T58132_P12 (SEQ ID NO: 216) | T58132_T2 (SEQ ID NO: 187) |
| T58132_P13 (SEQ ID NO: 217) | T58132_T11 (SEQ ID NO: 192); |
| | T58132_T12 (SEQ ID NO: 193) |

These sequences are variants of the known protein LEM domain containing 1a (SEQ ID NO: 205) (SwissProt accession identifier Q6L9U4_HUMAN), referred to herein as the previously known protein.

The following GO Annotation(s) apply to the previously known protein. The following annotation(s) were found: nuclear membrane, which are annotation(s) related to Cellular Component.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLinkl>.

Variant proteins T58132_P2 and T58132_P12 were previously disclosed (in published PCT application nos WO2005/071058 and WO2004/104161, respectively, both of which are hereby incorporated by reference as if fully set forth herein) but have now unexpectedly been found to have novel diagnostic properties, including but not limited to such properties for diagnosis of colon, lung and/or ovarian cancer.

As noted above, cluster T58132 features 7 transcript(s), which were listed in Table 129 above. These transcript(s) encode for protein(s) which are variant(s) of protein LEM domain containing 1A (SEQ ID NO: 205). A description of each variant protein according to the present invention is now provided.

Variant protein T58132_P0 (SEQ ID NO: 213) according to the present invention has an amino acid sequence as given in the “Sequence Listing” of the application; it is encoded by transcript(s) T58132_T4 (SEQ ID NO: 188). An alignment is given to the known protein (LEM domain containing 1A, SEQ ID NOs:205-212) in the alignment table, on the attached CDROM. One or more alignments to one or more previously published protein sequences are given in the alignment table, on the attached CDROM. A brief description of the relationship of the variant protein according to the present invention to each such aligned protein is as follows:

### 1. Comparison Report Between T58132_P0 (SEQ ID NO: 213) and Q6L9U4_HUMAN (SEQ ID NO:425):

A. An isolated chimeric polypeptide encoding for T58132_P0 (SEQ ID NO: 213), comprising a first amino acid sequence being at least about 70%, optionally at least 80%, preferably at least 85%, more preferably at least 90% and most preferably at least 95%, homologous to a polypeptide having the sequence AAWKAGSGSAGNCEADCEGKGALLSPPGFYHQKRGQTSII (SEQ ID NO: 508) corresponding to amino acids 1-40 of T58132_P0 (SEQ ID NO: 213), and a second amino acid sequence being at least about 90% homologous to MVDVKCLSDCKLQNQLEKLGFSPGPILPSTRKLYEKKLVQLLVSPPCAPPVMNGPRELDGAQDSDDSEEL NIILQGNIILSTEKSKKLKKWPEASTTKRKAVDTYCLDYKPSKGRRWAARAPSTRITYGTITKERDYCAED QTIESWREEGFPVGLKLAVLGIFIIVVFVYLTVENKSLFG corresponding to amino acids 1-181 of Q6L9U4_HUMAN (SEQ ID NO:425), which also corresponds to amino acids 41-221 of T58132_P0 (SEQ ID NO: 213), wherein said first amino acid sequence and second amino acid sequence are contiguous and in a sequential order.

B. An isolated polypeptide encoding for a head of T58132_P0 (SEQ ID NO: 213), comprising a polypeptide being at least 70%, optionally at least about 80%, preferably at least about 85%, more preferably at least about 90% and most preferably at least about 95% homologous to the sequence

(SEQ ID NO: 508)

AAWKAGSGSAGNCEADCEGKGALLSPPGFYHQKRGQTSII
of

(SEQ ID NO: 213)

T58132_P0.

### 2. Comparison Report Between T58132_P0 (SEQ ID NO: 213) and Q68G75_HUMAN (SEQ ID NO: 209):

A. An isolated chimeric polypeptide encoding for T58132_P0 (SEQ ID NO: 213), comprising a first amino acid sequence being at least about 70%, optionally at least 80%, preferably at least 85%, more preferably at least 90% and most preferably at least 95%, homologous to a polypeptide having the sequence AAWKAGSGSAGNCEADCEGKGALLSPPGFYHQKRGQTSII (SEQ ID NO: 508) corresponding to amino acids 1-40 of T58132_P0 (SEQ ID NO: 213), a second amino acid sequence being at least about 90% homologous to MVDVKCLSDCKLQNQLEKLGFSPGPILPSTRKLYEKKLVQLLVSPPCAPPVMNGPRELDGAQDSDDSEEL NIILQGNIILST corresponding to amino acids 1-82 of Q68G75_HUMAN (SEQ ID NO: 209), which also corresponds to amino acids 41-122 of T58132_P0 (SEQ ID NO: 213), a bridging amino acid E corresponding to amino acid 123 of T58132_P0 (SEQ ID NO: 213), and a third amino acid sequence being at least about 90% homologous to KSKKLLKKWPEASTTKRKAVDTYCLDYKPSKGRRWAARAPSTRITYGTITKERDYCAEDQTIESWREEGFP VGLKLAVLGIFIIVVFVYLTVENKSLFG corresponding to amino acids 84-181 of Q68G75_HUMAN (SEQ ID NO: 209), which also corresponds to amino acids 124-221 of T58132_P0 (SEQ ID NO: 213), wherein said first amino acid sequence, second amino acid sequence, bridging amino acid and third amino acid sequence are contiguous and in a sequential order.

### 3. Comparison Report Between T58132_P0 (SEQ ID NO: 213) and Q6L9U2_HUMAN (SEQ ID NO: 212):

A. An isolated chimeric polypeptide encoding for T58132_P0 (SEQ ID NO: 213), comprising a first amino acid sequence being at least about 70%, optionally at least 80%, preferably at least 85%, more preferably at least 90% and most preferably at least 95%, homologous to a polypeptide having the sequence AAWKAGSGSAGNCEADCEGKGALLSPPGFYHQKRGQTSII (SEQ ID NO: 508) corresponding to amino acids 1-40 of T58132_P0 (SEQ ID NO: 213), a second amino acid sequence being at least about 90% homologous to MVDVKCLSDCKLQNQLEKLGFSPGPIL corresponding to amino acids 1-27 of Q6L9U2_HUMAN (SEQ ID NO: 212), which also corresponds to amino acids 41-67 of T58132_P0 (SEQ ID NO: 213), a third amino acid sequence being at least about 70%, optionally at least 80%, preferably at least 85%, more preferably at least 90% and most preferably at least 95% homologous to a polypeptide having the sequence PSTRKLYEKKLVQLLVSPPCAPPVMNGPRELDGAQDSDDSEE (SEQ ID NO: 509) corresponding to amino acids 68-109 of T58132_P0 (SEQ ID NO: 213), and a fourth amino acid sequence being at least about 90% homologous to LNIILQGNIILSTEKSKKLKKWPEAST AVDTYCLDYKPSKGRRWAARAPSTRITYGTITKERD YCAEDQTIESWREEGFPVGLKLAVLGIFIIVVFVYLTVENKSLFG corresponding to amino acids 29-140 of Q6L9U2_HUMAN (SEQ ID NO: 212), which also corresponds to amino acids 110-221 of T58132_P0 (SEQ ID NO: 213), wherein said first amino acid sequence, second amino acid sequence, third amino acid sequence and fourth amino acid sequence are contiguous and in a sequential order.

C. An isolated polypeptide encoding for an edge portion of T58132_P0 (SEQ ID NO: 213), comprising an amino acid sequence being at least about 70%, optionally at least about 80%, preferably at least about 85%, more preferably at least about 90% and most preferably at least about 95% homologous to the sequence

(SEQ ID NO: 509)

PSTRKLYEKKLVQLLVSPPGAPPVMNGPRELDGAQDSDDSEE
of

(SEQ ID NO: 213)

T58132_P0.

The localization of the variant protein was determined according to results from a number of different software programs and analyses, including analyses from SignalP and other specialized programs. The variant protein is believed to be membranal.

Variant protein T58132_P0 (SEQ ID NO: 213) also has the following non-silent SNPs (Single Nucleotide Polymorphisms) as listed in Table 132, (given according to their position(s) on the amino acid sequence, with the alternative amino acid(s) listed).

TABLE 132
Amino acid mutations

| SNP position(s) on amino acid | |
|---|---|
| sequence | Alternative amino acid(s) |
| 139 | R -> C |

Variant protein T58132_P0 (SEQ ID NO: 213) is encoded by the following transcript(s): T58132_T4 (SEQ ID NO: 188), for which the coding portion starts at position 1 and ends at position 663. The transcript also has the following SNPs as listed in Table 133 (given according to their position on the nucleotide sequence, with the alternative nucleic acid listed).

TABLE 133
Nucleic acid SNPs

| | SNP position(s) on nucleotide |
|---|---|
| Polymorphism | sequence |
| C -> T | 415 |

Variant protein T58132_P1 (SEQ ID NO: 214) according to the present invention has an amino acid sequence as given in the “Sequence Listing” of the application; it is encoded by transcript(s) T58132_T7 (SEQ ID NO: 189). An alignment is given to the known protein (LEM domain containing 1A, SEQ ID NOs:205-212) in the alignment table, on the attached CDROM. One or more alignments to one or more previously published protein sequences are given in the alignment table, on the attached CDROM. A brief description of the relationship of the variant protein according to the present invention to each such aligned protein is as follows:

### 1. Comparison Report Between T58132_P1 (SEQ ID NO: 214) and NP_{—}001001552 (SEQ ID NO: 206) (or Q6L9UO_HUMAN (SEQ ID NO: 210)):

A. An isolated chimeric polypeptide encoding for T58132_P1 (SEQ ID NO: 214), comprising a first amino acid sequence being at least about 70%, optionally at least 80%, preferably at least 85%, more preferably at least 90% and most preferably at least 95%, homologous to a polypeptide having the sequence AAWKAGSGSAGNCEADCEGKGALLSPPGFYFIQKRGQTSII (SEQ ID NO: 508) corresponding to amino acids 1-40 of T58132_P1 (SEQ ID NO: 214), and a second amino acid sequence being at least about 90% homologous to MVDVKCLSDCKLQNQLEKLGFSPGPILPSTRKLYEKKLVQLLVSPPCAPPVMNGPRELDGAQDSDDSEGG LQEHQAPESHMGLSPKRETTARKTRLSRAGEKKVSQWA corresponding to amino acids 1-108 of NP_{—}001001552 (SEQ ID NO: 206), which also corresponds to amino acids 41-148 of T58132_P1 (SEQ ID NO: 214), wherein said first amino acid sequence and second amino acid sequence are contiguous and in a sequential order.

The localization of the variant protein was determined according to results from a number of different software programs and analyses, including analyses from SignalP and other specialized programs. The variant protein is believed to be located intracellularly with regard to the cell.

Variant protein T58132_P1 (SEQ ID NO: 214) is encoded by the following transcript(s): T58132_T7 (SEQ ID NO: 189), for which the coding portion starts at position 1 and ends at position 444.

Variant protein T58132_P2 (SEQ ID NO: 215) according to the present invention has an amino acid sequence as given in the “Sequence Listing” of the application; it is encoded by transcript(s) T58132_T10 (SEQ ID NO: 191) and T58132_T9 (SEQ ID NO: 190).

The localization of the variant protein was determined according to results from a number of different software programs and analyses, including analyses from SignalP and other specialized programs. The variant protein is believed to be located intracellularly with regard to the cell.

Variant protein T58132_P2 (SEQ ID NO: 215) is encoded by the following transcript(s): T58132_T10 (SEQ ID NO: 191) and T58132_T9 (SEQ ID NO: 190), for which the sequence(s) is/are given in the “Sequence Listing” of the application.

The coding portion of transcript T58132_T10 (SEQ ID NO: 191) starts at position 637 and ends at position 957. The transcript also has the following SNPs as listed in Table 134 (given according to their position on the nucleotide sequence, with the alternative nucleic acid listed; T58132_P2 (SEQ ID NO: 215)).

TABLE 134
Nucleic acid SNPs

| | SNP position(s) on nucleotide |
|---|---|
| Polymorphism | sequence |
| T -> | 432 |
| C -> T | 460, 1878 |
| A -> G | 1067 |
| G -> A | 1547 |
| C -> A | 1739 |

The coding portion of transcript T58132_T9 (SEQ ID NO: 190) starts at position 1400 and ends at position 1720. The transcript also has the following SNPs as listed in Table 135 (given according to their position on the nucleotide sequence, with the alternative nucleic acid listed).

TABLE 135
Nucleic acid SNPs

| | SNP position(s) on nucleotide |
|---|---|
| Polymorphism | sequence |
| T -> | 1195 |
| C -> T | 1223, 2641 |
| A -> G | 1830 |
| G -> A | 2310 |
| C -> A | 2502 |

Variant protein T58132_P12 (SEQ ID NO: 216) according to the present invention has an amino acid sequence as given in the “Sequence Listing” of the application; it is encoded by transcript(s) T58132_T2 (SEQ ID NO: 187). An alignment is given to the known protein (LEM domain containing 1A, SEQ ID NOs:205-212, 425) in the alignment table, on the attached CDROM. One or more alignments to one or more previously published protein sequences are given in the alignment table, on the attached CDROM. A brief description of the relationship of the variant protein according to the present invention to each such aligned protein is as follows:

### 1. Comparison Report Between T58132_P12 (SEQ ID NO: 216) and Q6L9U4_HUMAN (SEQ ID NO:425):

A. An isolated chimeric polypeptide encoding for T58132_P12 (SEQ ID NO: 216), comprising a first amino acid sequence being at least about 90% homologous to MVDVKCLSDCKLQNQLEKLGFSPGPIL corresponding to amino acids 1-27 of Q6L9U4_HUMAN (SEQ ID NO:425), which also corresponds to amino acids 1-27 of T58132_P12 (SEQ ID NO: 216), a second bridging amino acid sequence comprising of Q, and a third amino acid sequence being at least about 90% homologous to LNIILQGNIILSTEKSKKLKKWPEASTTKRKAVDTYCLDYKPSKGRRWAARAPSTRITYGTITKERDYCAED QTIESWREEGFPVGLKLAVLGIFIIVVFVYLTVENKSLFG corresponding to amino acids 70-181 of Q6L9U4_HUMAN (SEQ ID NO:425), which also corresponds to amino acids 29-140 of T58132_P12 (SEQ ID NO: 216), wherein said first amino acid sequence, second amino acid sequence and third amino acid sequence are contiguous and in a sequential order.

B. An isolated polypeptide encoding for an edge portion of T58132_P12 (SEQ ID NO: 216), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least 3 amino acids comprise LQL having a structure as follows (numbering according to T58132_P12 (SEQ ID NO: 216)): a sequence starting from any of amino acid numbers 27−x to 27; and ending at any of amino acid numbers 29+((n−3)−x), in which x varies from 0 to n-3.

### 2. Comparison Report Between T58132_P12 (SEQ ID NO: 216) and Q68G75_HUMAN (SEQ ID NO: 209):

A. An isolated chimeric polypeptide encoding for T58132_P12 (SEQ ID NO: 216), comprising a first amino acid sequence being at least about 90% homologous to MVDVKCLSDCKLQNQLEKLGFSPGPIL corresponding to amino acids 1-27 of Q68G75_HUMAN (SEQ ID NO: 209), which also corresponds to amino acids 1-27 of T58132_P12 (SEQ ID NO: 216), a second bridging amino acid sequence comprising of Q, a third amino acid sequence being at least about 90% homologous to LNIILQGNIILST corresponding to amino acids 70-82 of Q68G75_HUMAN (SEQ ID NO: 209), which also corresponds to amino acids 29-41 of T58132_P12 (SEQ ID NO: 216), a bridging amino acid E corresponding to amino acid 42 of T58132_P12 (SEQ ID NO: 216), and a fourth amino acid sequence being at least about 90% homologous to KSKKLKKWPEASTTKRKAVDTYCLDYKPSKGRRWAARAPSTRITYGTITKERDYCAEDQTIESWREEGFP VGLKLAVLGIFIIVVFVYLTVENKSLFG corresponding to amino acids 84-181 of Q68G75_HUMAN (SEQ ID NO: 209), which also corresponds to amino acids 43-140 of T58132_P12 (SEQ ID NO: 216), wherein said first amino acid sequence, second amino acid sequence, third amino acid sequence, bridging amino acid and fourth amino acid sequence are contiguous and in a sequential order.

The localization of the variant protein was determined according to results from a number of different software programs and analyses, including analyses from SignalP and other specialized programs. The variant protein is believed to be membranal.

Variant protein T58132_P12 (SEQ ID NO: 216) also has the following non-silent SNPs (Single Nucleotide Polymorphisms) as listed in Table 136, (given according to their position(s) on the amino acid sequence, with the alternative amino acid(s) listed).

TABLE 136
Amino acid mutations

| SNP position(s) on amino acid | |
|---|---|
| sequence | Alternative amino acid(s) |
| 58 | R -> C |

Variant protein T58132_P12 (SEQ ID NO: 216) is encoded by the following transcript(s): T58132_T2 (SEQ ID NO: 187), for which the sequence(s) is/are given in the “Sequence Listing” of the application. The coding portion of transcript T58132_T2 (SEQ ID NO: 187) starts at position 103 and ends at position 522. The transcript also has the following SNPs as listed in Table 137 (given according to their position on the nucleotide sequence, with the alternative nucleic acid listed).

TABLE 137
Nucleic acid SNPs

| | SNP position(s) on nucleotide |
|---|---|
| Polymorphism | sequence |
| C -> T | 274 |

Variant protein T58132_P13 (SEQ ID NO: 217) according to the present invention has an amino acid sequence as given in the “Sequence Listing” of the application; it is encoded by transcript(s) T58132_T11 (SEQ ID NO: 192) and T58132_T12 (SEQ ID NO: 193).

The localization of the variant protein was determined according to results from a number of different software programs and analyses, including analyses from SignalP and other specialized programs. The variant protein is believed to be located intracellularly with regard to the cell.

Variant protein T58132_P13 (SEQ ID NO: 217) also has the following non-silent SNPs (Single Nucleotide Polymorphisms) as listed in Table 138, (given according to their position(s) on the amino acid sequence, with the alternative amino acid(s) listed).

TABLE 138
Amino acid mutations

| SNP position(s) on amino acid | |
|---|---|
| sequence | Alternative amino acid(s) |
| 46 | S -> L |

Variant protein T58132_P13 (SEQ ID NO: 217) is encoded by the following transcript(s): T58132_T11 (SEQ ID NO: 192) and T58132_T12 (SEQ ID NO: 193), for which the coding portion starts at position 903 and ends at position 1133. The transcript also has the following SNPs as listed in Table 139 (given according to their position on the nucleotide sequence, with the alternative nucleic acid listed).

TABLE 139
Nucleic acid SNPs

| | SNP position(s) on nucleotide |
|---|---|
| Polymorphism | sequence |
| G -> A | 658 |
| C -> T | 1039 |

The coding portion of transcript T58132_T12 (SEQ ID NO: 193) starts at position 887 and ends at position 1117. The transcript also has the following SNPs as listed in Table 140 (given according to their position on the nucleotide sequence, with the alternative nucleic acid listed).

TABLE 140
Nucleic acid SNPs

| | SNP position(s) on nucleotide |
|---|---|
| Polymorphism | sequence |
| G -> A | 642 |
| C -> T | 1023 |

As noted above, cluster T58132 features 11 segment(s), which were listed in Table 130 above and for which the sequence(s) are given in the “Sequence Listing” of the application. These segment(s) are portions of nucleic acid sequence(s) which are described herein separately because they are of particular interest. A description of some of these segments according to the present invention is now provided.

Segment cluster T58132_N6 (SEQ ID NO: 194) according to the present invention is supported by 3 libraries. The number of libraries was determined as previously described. This segment can be found in the following transcript(s): T58132_T11 (SEQ ID NO: 192) and T58132_T12 (SEQ ID NO: 193). Table 141 below describes the starting and ending position of this segment on each transcript.

TABLE 141
Segment location on transcripts

| | Segment | Segment |
|---|---|---|
| Transcript name | starting position | ending position |
| T58132_T11 (SEQ ID NO: 192) | 83 | 1225 |
| T58132_T12 (SEQ ID NO: 193) | 67 | 1209 |

Segment cluster T58132_N8 (SEQ ID NO: 195) according to the present invention is supported by 6 libraries. The number of libraries was determined as previously described. This segment can be found in the following transcript(s): T58132 T9 (SEQ ID NO: 190). Table 142 below describes the starting and ending position of this segment on each transcript.

TABLE 142
Segment location on transcripts

| | Segment | Segment |
|---|---|---|
| Transcript name | starting position | ending position |
| T58132_T9 (SEQ ID NO: 190) | 1 | 845 |

Segment cluster T58132_N9 (SEQ ID NO: 196) according to the present invention is supported by 21 libraries. The number of libraries was determined as previously described. This segment can be found in the following transcript(s): T58132_T10 (SEQ ID NO: 191) and T58132_T9 (SEQ ID NO: 190). Table 143 below describes the starting and ending position of this segment on each transcript.

TABLE 143
Segment location on transcripts

| | Segment | Segment |
|---|---|---|
| Transcript name | starting position | ending position |
| T58132_T10 (SEQ ID NO: 191) | 83 | 2411 |
| T58132_T9 (SEQ ID NO: 190) | 846 | 3174 |

Segment cluster T58132_N13 (SEQ ID NO: 197) according to the present invention is supported by 17 libraries. The number of libraries was determined as previously described. This segment can be found in the following transcript(s): T58132_T2 (SEQ ID NO: 187), T58132_T4 (SEQ ID NO: 188) and T58132_T7 (SEQ ID NO: 189). Table 144 below describes the starting and ending position of this segment on each transcript.

TABLE 144
Segment location on transcripts

| | Segment | Segment |
|---|---|---|
| Transcript name | starting position | ending position |
| T58132_T2 (SEQ ID NO: 187) | 65 | 184 |
| T58132_T4 (SEQ ID NO: 188) | 83 | 202 |
| T58132_T7 (SEQ ID NO: 189) | 83 | 202 |

Segment cluster T58132_N15 (SEQ ID NO: 198) according to the present invention is supported by 7 libraries. The number of libraries was determined as previously described. This segment can be found in the following transcript(s): T58132_T4 (SEQ ID NO: 188) and T58132_T7 (SEQ ID NO: 189). Table 145 below describes the starting and ending position of this segment on each transcript.

TABLE 145
Segment location on transcripts

| | Segment | Segment |
|---|---|---|
| Transcript name | starting position | ending position |
| T58132_T4 (SEQ ID NO: 188) | 203 | 325 |
| T58132_T7 (SEQ ID NO: 189) | 203 | 325 |

Segment cluster T58132_N21 (SEQ ID NO: 199) according to the present invention is supported by 28 libraries. The number of libraries was determined as previously described. This segment can be found in the following transcript(s): T58132_T2 (SEQ ID NO: 187), T58132_T4 (SEQ ID NO: 188) and T58132_T7 (SEQ ID NO: 189). Table 146 below describes the starting and ending position of this segment on each transcript.

TABLE 146
Segment location on transcripts

| | Segment | Segment |
|---|---|---|
| Transcript name | starting position | ending position |
| T58132_T2 (SEQ ID NO: 187) | 327 | 805 |
| T58132_T4 (SEQ ID NO: 188) | 468 | 946 |
| T58132_T7 (SEQ ID NO: 189) | 326 | 804 |

According to an optional embodiment of the present invention, short segments related to the above cluster are also provided. These segments are up to about 120 bp in length, and so are included in a separate description.

Segment cluster T58132_N2 (SEQ ID NO: 200) according to the present invention is supported by 4 libraries. The number of libraries was determined as previously described. This segment can be found in the following transcript(s): T58132_T10 (SEQ ID NO: 191), T58132_T11 (SEQ ID NO: 192), T58132 T4 (SEQ ID NO: 188) and T58132_T7 (SEQ ID NO: 189). Table 147 below describes the starting and ending position of this segment on each transcript.

TABLE 147
Segment location on transcripts

| | Segment | Segment |
|---|---|---|
| Transcript name | starting position | ending position |
| T58132_T10 (SEQ ID NO: 191) | 1 | 82 |
| T58132_T11 (SEQ ID NO: 192) | 1 | 82 |
| T58132_T4 (SEQ ID NO: 188) | 1 | 82 |
| T58132_T7 (SEQ ID NO: 189) | 1 | 82 |

Segment cluster T58132_N4 (SEQ ID NO: 201) according to the present invention is supported by 1 libraries. The number of libraries was determined as previously described. This segment can be found in the following transcript(s): T58132_T12 (SEQ ID NO: 193). Table 148 below describes the starting and ending position of this segment on each transcript.

TABLE 148
Segment location on transcripts

| | Segment | Segment |
|---|---|---|
| Transcript name | starting position | ending position |
| T58132_T12 (SEQ ID NO: 193) | 1 | 66 |

Segment cluster T58132_N17 (SEQ ID NO: 203) according to the present invention is supported by 4 libraries. The number of libraries was determined as previously described. This segment can be found in the following transcript(s): T58132_T2 (SEQ ID NO: 187) and T58132_T4 (SEQ ID NO: 188). Table 149 below describes the starting and ending position of this segment on each transcript.

TABLE 149
Segment location on transcripts

| | Segment | Segment |
|---|---|---|
| Transcript name | starting position | ending position |
| T58132_T2 (SEQ ID NO: 187) | 185 | 249 |
| T58132_T4 (SEQ ID NO: 188) | 326 | 390 |

Expression of Homo sapiens LEM domain containing 1 (LEMD1) transcripts detectable by or according to segments as described below was measured by real time PCR: seg6F2R—T58132_seg6F2R2 (SEQ ID NO: 273) amplicon and primers T58132_seg6F2 (SEQ ID NO: 271) and T58132_seg6R2 (SEQ ID NO: 272); seg8F2R2—T58132_seg8F2R2 (SEQ ID NO: 276) amplicon and primers T58132_seg8F2 (SEQ ID NO: 274) and T58132_seg8R2 (SEQ ID NO: 275); seg21—T58132_seg21 (SEQ ID NO: 279) amplicon and primers T58132_seg21F (SEQ ID NO: 277) and T58132_seg21R (SEQ ID NO: 278).

The sequences of the corresponding amplicons and primers are given below:

Forward Primer (T58132_seg6F2 (SEQ ID) NO: 271)):
CCCCAGTTCACTTTCGCCT
Reverse Primer (T58132_seg6R2 (SEQ ID NO: 272)):
TCTGATCACACCGCCGTCT
Amplicon (T58132_seg6F2R2 (SEQ ID NO: 273)):
CCCCAGTTCACTTTCGCCTGCTTCTGTGGTCTGCACGGCTTCTGCAAAAT
GAAGAGGAAGAAGGAGGAAGTTCACAGAGAGCGGGAGACGGCGGTGTGAT
CAGA
Forward Primer (T58132_seg8F2 (SEQ ID NO: 274)):
CCCAGAGCTTCTGCTAGAATCTTC
Reverse Primer (T58132_seg8R2 (SEQ ID NO: 275)):
ACTGACTTACTCCCCTTGGGC
Amplicon (T58132_seg8F2R2 (SEQ ID NO: 276)):
CCCAGAGCTTCTGCTAGAATCTTCCCCTAGCCAGGGCCCAGGACTCCTTG
CAGTTCTGCCTCTCTTCCCTCCTACAGCCAGGCCCAAGGGGAGTAAGTCA
GT
Forward Primer (T58132_seg21F (SEQ ID NO: 277)):
GAGCACCAAGCACCAGAATCA
Reverse Primer (T58132_seg21R (SEQ ID NO: 278)):
TGGGAAACCTTCTTCTCTCCAG
Amplicon (T58132_seg21 (SEQ ID NO: 279)):
AGCACCAAGCACCAGAATCACATATGGGACTATCACCAAAGAGAGAGACT
ACTGCGCGGAAGACCAGACTATCGAGAGCTGGAGAGAAGAAGGTTTCCCA

Expression of Homo sapiens LEM Domain Containing 1 (LEMD1) T58132 Transcripts which are Detectable by Amplicon as Depicted in Sequence Name T58132_seg6F2R2 (SEQ ID NO: 273) in Normal and Cancerous Colon Tissues.

Expression of Homo sapiens LEM domain containing 1 (LEMD1) transcripts detectable by or according to seg6F2R2—T58132_seg6F2R2 (SEQ ID NO: 273) amplicon and primers T58132_seg6F2 (SEQ ID NO: 271) and T58132_seg6R2 (SEQ ID NO: 272) was measured by real time PCR. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), RPS27a (GenBank Accession No. NM_{—}002954 (SEQ ID NO: 340); RPS27A amplicon) and G6PD (GenBank Accession No. NM_{—}000402 (SEQ ID NO:339); G6PD amplicon (SEQ ID NO: 311)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 41, 52, 62, 63, 64, 65, 66, 67, 69 and 71, Table 1_{—}4 above: “Tissue samples in colon cancer testing panel”), to obtain a value of fold up-regulation for each sample relative to the median of the normal PM samples.

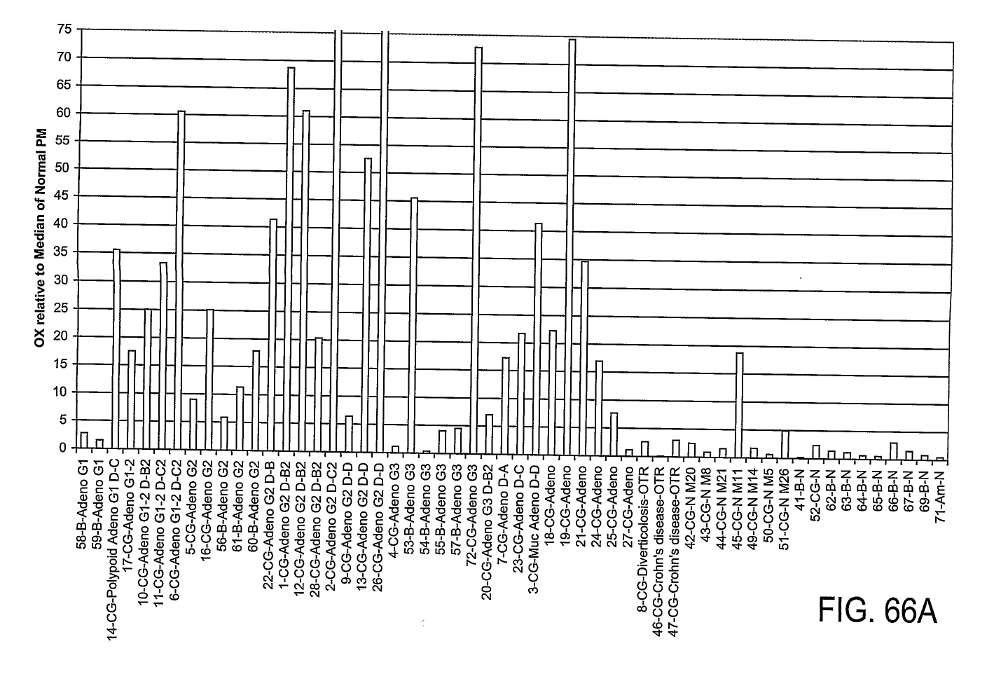

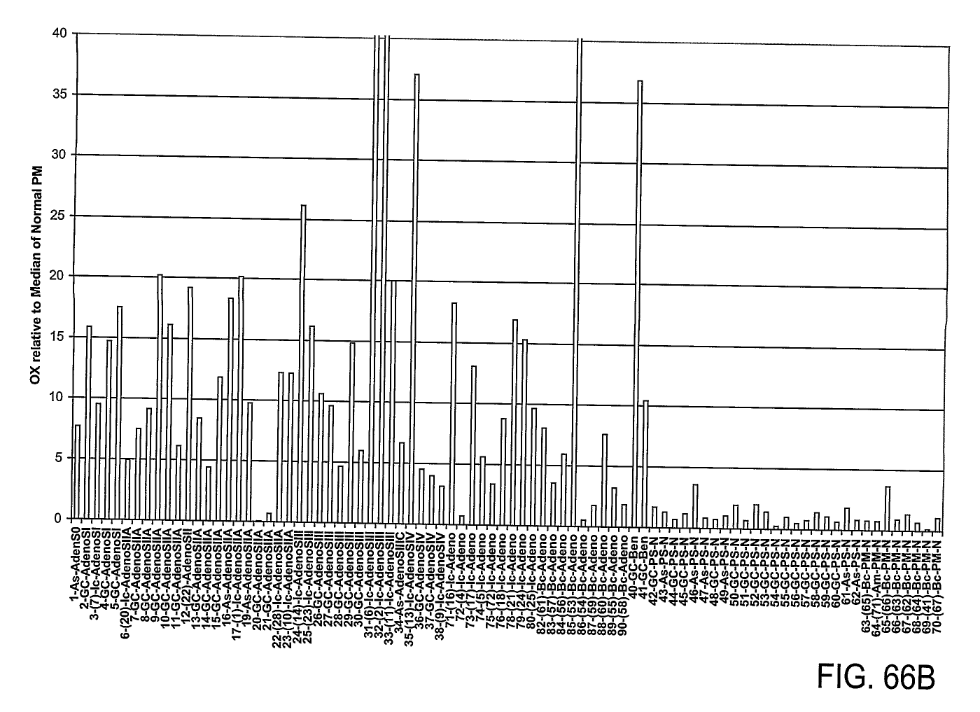

FIG. 66A is a histogram showing over expression of the Homo sapiens LEM domain containing 1 (LEMD1) transcripts in cancerous Colon samples relative to the normal samples.

As is evident from FIG. 66A, the expression of Homo sapiens LEM domain containing 1 (LEMD1) transcripts detectable by the above amplicon in cancer samples was significantly higher than in the non-cancerous samples (sample numbers 41, 52, 62, 63, 64, 65, 66, 67, 69 and 71, Table 1_{—}4 above: “Tissue samples in colon cancer testing panel”). Notably an over-expression of at least 5 fold was found in 29 out of 36 adenocarcinoma samples.

Statistical analysis was applied to verify the significance of these results, as described below.

The P value for the difference in the expression levels of Homo sapiens LEM domain containing 1 (LEMD1) transcripts detectable by the above amplicon in Colon cancer samples versus the normal tissue samples was determined by T test as 2.51e-007.

Threshold of 5 fold over expression was found to differentiate between cancer and normal samples with P value of 4.77e-006 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T58132_seg6F2 (SEQ ID NO: 271) forward primer; and T58132_seg6R2 (SEQ ID NO: 272) reverse primer.

The present invention also preferably encompasses any amplicon obtained through the use of any suitable primer pair; for example, for the above experiment, the following amplicon was obtained as a non-limiting illustrative example only of a suitable amplicon: T58132_seg6F2R2 (SEQ ID NO: 273).

Expression of Homo sapiens LEM domain containing 1 (LEMD1) transcripts detectable by or according to seg6F2R2—T58132_seg6F2R2 (SEQ ID NO: 273) amplicon and primers T58132_seg6F2 (SEQ ID NO: 271) and T58132_seg6R2 (SEQ ID NO: 272) was further measured by real time PCR using enlarged panel as shown in Table 1_{—}8. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), and G6PD (GenBank Accession No. NM_{—}000402 (SEQ ID NO:339); G6PD amplicon (SEQ ID NO: 311)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 42, 43, 44, 45, 46, 47, 48, 49, 50, 51, 52, 53, 54, 55, 56, 57, 58, 59, 60, 61, 62, 63, 64, 65, 66, 67, 68, 69 and 70, Table 1_{—}8 above), to obtain a value of fold up-regulation for each sample relative to the median of the normal samples.

FIG. 66B is a histogram showing over expression of the Homo sapiens LEM domain containing 1 (LEMD1) transcripts in cancerous Colon samples relative to the normal samples.

As is evident from FIG. 66B, the expression of Homo sapiens LEM domain containing 1 (LEMD1) transcripts detectable by the above amplicon in cancer samples was significantly higher than in the normal samples (sample numbers 42, 43, 44, 45, 46, 47, 48, 49, 50, 51, 52, 53, 54, 55, 56, 57, 58, 59, 60, 61, 62, 63, 64, 65, 66, 67, 68, 69 and 70, Table 1_{—}8 above). Notably an over-expression of at least 5 fold was found in 40 out of 55 adenocarcinoma samples.

Statistical analysis was applied to verify the significance of these results, as described below.

The P value for the difference in the expression levels of Homo sapiens LEM domain containing 1 (LEMD1) transcripts detectable by the above amplicon in Colon cancer samples versus the normal tissue samples was determined by T test as 1.81 e-08 Threshold of 5 fold over expression was found to differentiate between cancer and normal samples with P value of 7.79e-12 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T58132_seg6F2 (SEQ ID NO: 271) forward primer; and T58132_seg6R2 (SEQ ID NO: 272) reverse primer.

The present invention also preferably encompasses any amplicon obtained through the use of any suitable primer pair; for example, for the above experiment, the following amplicon was obtained as a non-limiting illustrative example only of a suitable amplicon: T58132_seg6F2R2 (SEQ ID NO: 273).

Expression of Homo sapiens LEM Domain Containing 1 (LEMD1) T58132 Transcripts which are Detectable by Amplicon as Depicted in Sequence Name T58132_seg6F2R2 (SEQ ID NO: 273) in Different Normal Tissues.

Expression of Homo sapiens LEM domain containing 1 (LEMD1) transcripts detectable by or according to seg6F2R2—T58132_seg6F2R2 (SEQ ID NO: 273) amplicon and primers T58132_seg6F2 (SEQ ID NO: 271) and T58132_seg6R2 (SEQ ID NO: 272) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the geometric mean of the quantities of the housekeeping genes. The normalized quantity of each RT sample was then divided by the median of the quantities of the colon samples (sample numbers 1, 2 and 3, Table 1_{—}9 above: “Tissue samples in normal panel”), to obtain a value of relative expression of each sample relative to the median of the colon samples.

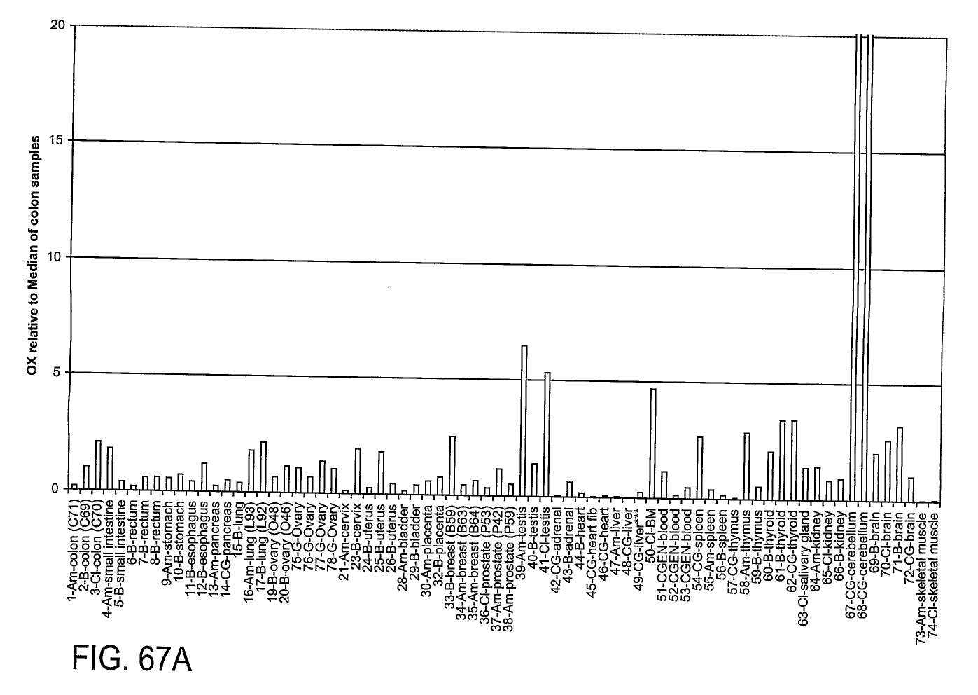

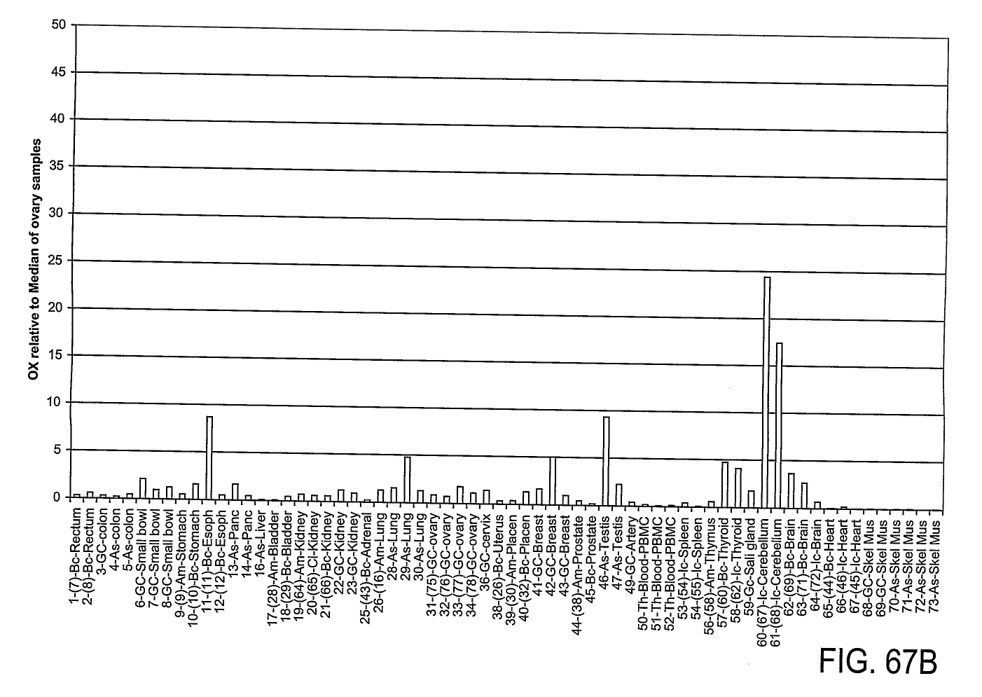

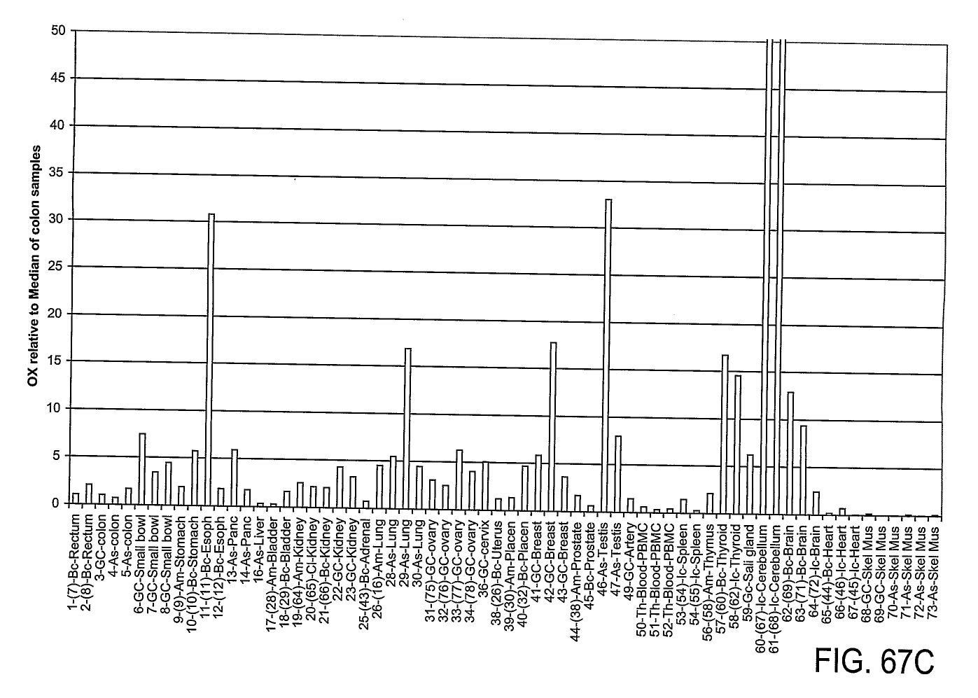

FIG. 67A is a histogram showing expression of Homo sapiens LEM domain containing 1 (LEMD1) T58132 transcripts which are detectable by amplicon as depicted in sequence name T58132_seg6F2R2 (SEQ ID NO: 273) in different normal tissues.

Expression of Homo sapiens LEM domain containing 1 (LEMD1) transcripts detectable by or according to seg6F2R2—T58132_seg6F2R2 (SEQ ID NO: 273) amplicon and primers T58132_seg6F2 (SEQ ID NO: 271) and T58132_seg6R2 (SEQ ID NO: 272) was further measured by real time PCR using updated panel as shown in Table 1_{—}10. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), and TATA box (GenBank Accession No. NM_{—}003194 (SEQ ID NO:343); TATA amplicon (SEQ ID NO: 323)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the ovary samples (sample numbers 31, 32, 33 and 34, Table 1_{—}9 above), to obtain a value of relative expression of each sample relative to the median of the ovary samples as presented in FIG. 67B. The normalized quantity of each RT sample was also divided by the median of the quantities of the colon samples (sample numbers 3, 4, 5, Table 1_{—}4 above), to obtain a value of relative expression of each sample relative to the median of the colon samples as presented in FIG. 67C.

Expression of Homo sapiens LEM Domain Containing 1 (LEMD1) T58132 Transcripts which are Detectable by Amplicon as Depicted in Sequence Name T58132_seg8F2R (SEQ ID NO: 276) in Normal and Cancerous Colon Tissues.

Expression of Homo sapiens LEM domain containing 1 (LEMD1) transcripts detectable by or according to seg8F2R2—T58132_seg8F2R2 (SEQ ID NO: 276) amplicon and primers T58132_seg8F2 (SEQ ID NO: 274) and T58132_seg8R2 (SEQ ID NO: 275) was measured by real time PCR. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), RPS27a (GenBank Accession No. NM_{—}002954 (SEQ ID NO: 340); RPS27A amplicon) and G6PD (GenBank Accession No. NM_{—}000402 (SEQ ID NO:339); G6PD amplicon (SEQ ID NO: 311)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 41, 52, 62, 63, 64, 65, 66, 67, 69 and 71, Table 1_{—}4 above, “Tissue samples in colon cancer testing panel”), to obtain a value of fold up-regulation for each sample relative to the median of the normal PM samples.

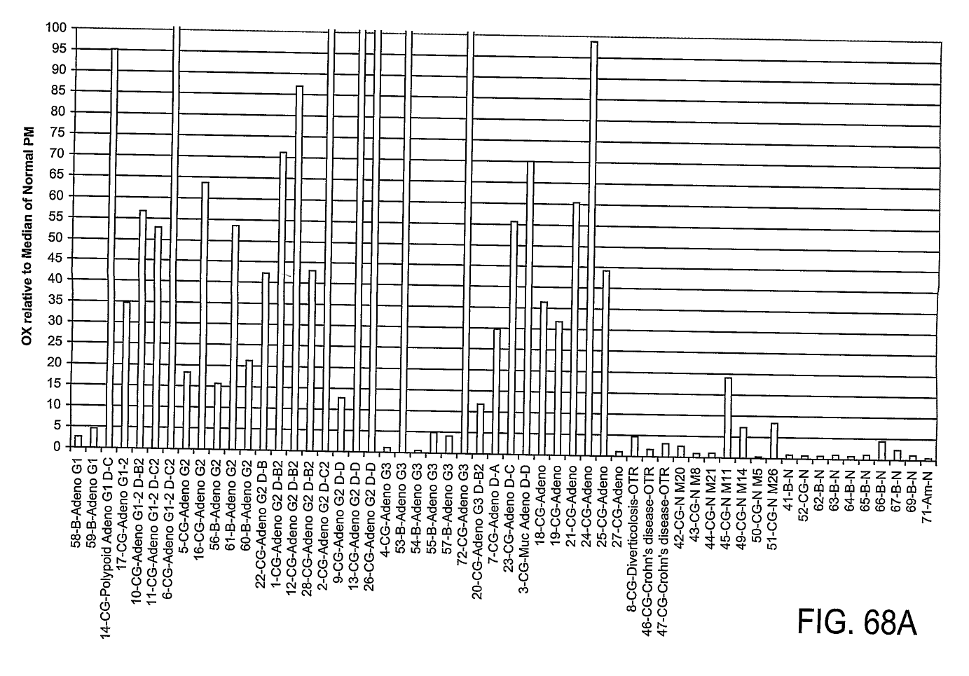

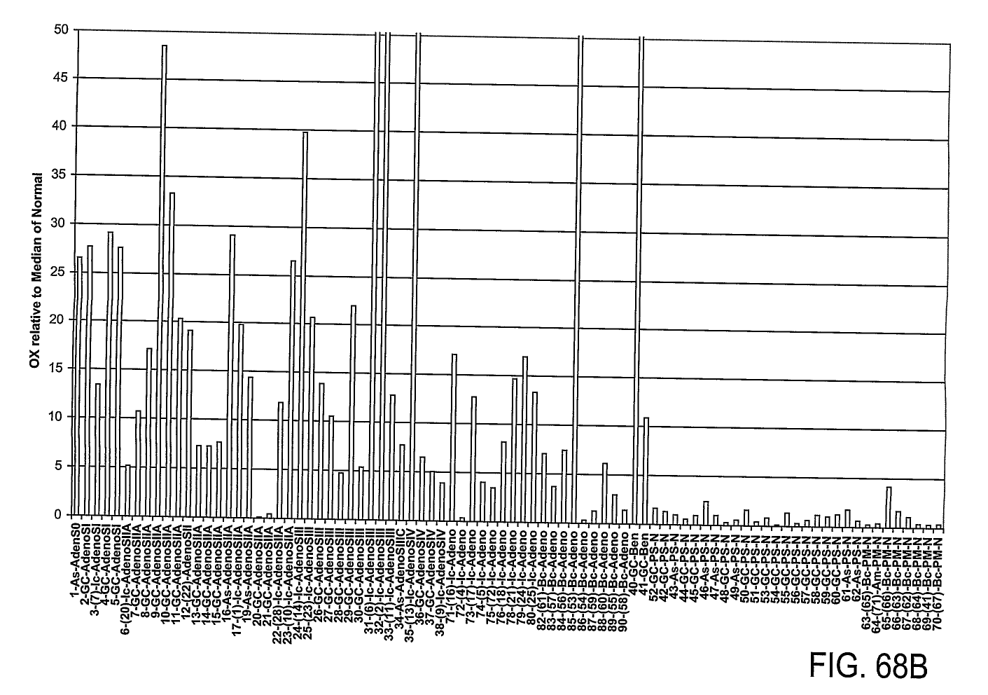

FIG. 68A is a histogram showing over expression of the Homo sapiens LEM domain containing 1 (LEMD1) transcripts in cancerous Colon samples relative to the normal samples.

As is evident from FIG. 68A, the expression of Homo sapiens LEM domain containing 1 (LEMD1) transcripts detectable by the above amplicon in cancer samples was significantly higher than in the non-cancerous samples (sample numbers 41, 52, 62, 63, 64, 65, 66, 67, 69 and 71, Table 1_{—}4 above: “Tissue samples in colon cancer testing panel”). Notably an over-expression of at least 5 fold was found in 29 out of 36 adenocarcinoma samples.

Statistical analysis was applied to verify the significance of these results, as described below.

The P value for the difference in the expression levels of Homo sapiens LEM domain containing 1 (LEMD1) transcripts detectable by the above amplicon in Colon cancer samples versus the normal tissue samples was determined by T test as 9.17e-007.

Threshold of 5 fold over expression was found to differentiate between cancer and normal samples with P value of 4.77e-006 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T58132_seg8F2 (SEQ ID NO: 274) forward primer; and T58132_seg8R2 (SEQ ID NO: 275) reverse primer.

The present invention also preferably encompasses any amplicon obtained through the use of any suitable primer pair; for example, for the above experiment, the following amplicon was obtained as a non-limiting illustrative example only of a suitable amplicon: T58132_seg8F2R2 (SEQ ID NO: 276).

Expression of Homo sapiens LEM domain containing 1 (LEMD1) transcripts detectable by or according to seg8F2R2—T58132_seg8F2R2 (SEQ ID NO: 276) amplicon and primers T58132_seg8F2 (SEQ ID NO: 274) and T58132_seg8R2 (SEQ ID NO: 275) was further measured by real time PCR using enlarged panel as shown in Table 1_{—}8. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), and G6PD (GenBank Accession No. NM_{—}000402 (SEQ ID NO:339); G6PD amplicon (SEQ ID NO: 311)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 52, 42, 43, 44, 45, 46, 47, 48, 49, 50, 51, 53, 54, 55, 56, 57, 58, 59, 60, 61, 62, 63, 64, 65, 66, 67, 68, 69 and 70, Table 1_{—}8 above), to obtain a value of fold up-regulation for each sample relative to the median of the normal samples.

FIG. 68B is a histogram showing over expression of the Homo sapiens LEM domain containing 1 (LEMD1) transcripts in cancerous Colon samples relative to the normal samples.

As is evident from FIG. 68B, the expression of Homo sapiens LEM domain containing 1 (LEMD1) transcripts detectable by the above amplicon in cancer samples was significantly higher than in the non-cancerous samples (sample numbers 52, 42, 43, 44, 45, 46, 47, 48, 49, 50, 51, 53, 54, 55, 56, 57, 58, 59, 60, 61, 62, 63, 64, 65, 66, 67, 68, 69 and 70, Table 1_{—}8 above). Notably an over-expression of at least 5 fold was found in 43 out of 55 adenocarcinoma samples.

Statistical analysis was applied to verify the significance of these results, as described below.

The P value for the difference in the expression levels of Homo sapiens LEM domain containing 1 (LEMD1) transcripts detectable by the above amplicon in Colon cancer samples versus the normal tissue samples was determined by T test as 2.73e-07 Threshold of 5 fold over expression was found to differentiate between cancer and normal samples with P value of 2.68e-13 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T58132_seg8F2 (SEQ ID NO: 274) forward primer; and T58132_seg8R2 (SEQ ID NO: 275) reverse primer.

The present invention also preferably encompasses any amplicon obtained through the use of any suitable primer pair; for example, for the above experiment, the following amplicon was obtained as a non-limiting illustrative example only of a suitable amplicon: T58132_seg8F2R2 (SEQ ID NO: 276).

Expression of Homo sapiens LEM Domain Containing 1 (LEMD1) T58132 Transcripts which are Detectable by Amplicon as Depicted in Sequence Name T58132_seg21 (SEQ ID NO: 279) in Normal and Cancerous Colon Tissues.

Expression of Homo sapiens LEM domain containing 1 (LEMD1) transcripts detectable by or according to seg21_T58132_seg21 (SEQ ID NO: 279) amplicon and primers T58132_seg21F (SEQ ID NO: 277) and T58132_seg21R (SEQ ID NO: 278) was measured by real time PCR. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), RPS27a (GenBank Accession No. NM_{—}002954 (SEQ ID NO: 340); RPS27A amplicon) and G6PD (GenBank Accession No. NM_{—}000402 (SEQ ID NO:339); G6PD amplicon (SEQ ID NO: 311)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 41, 63, 64, 65, 66, 69 and 71, Table 1_{—}4 above: “Tissue samples in colon cancer testing panel”), to obtain a value of fold up-regulation for each sample relative to the median of the normal PM samples.

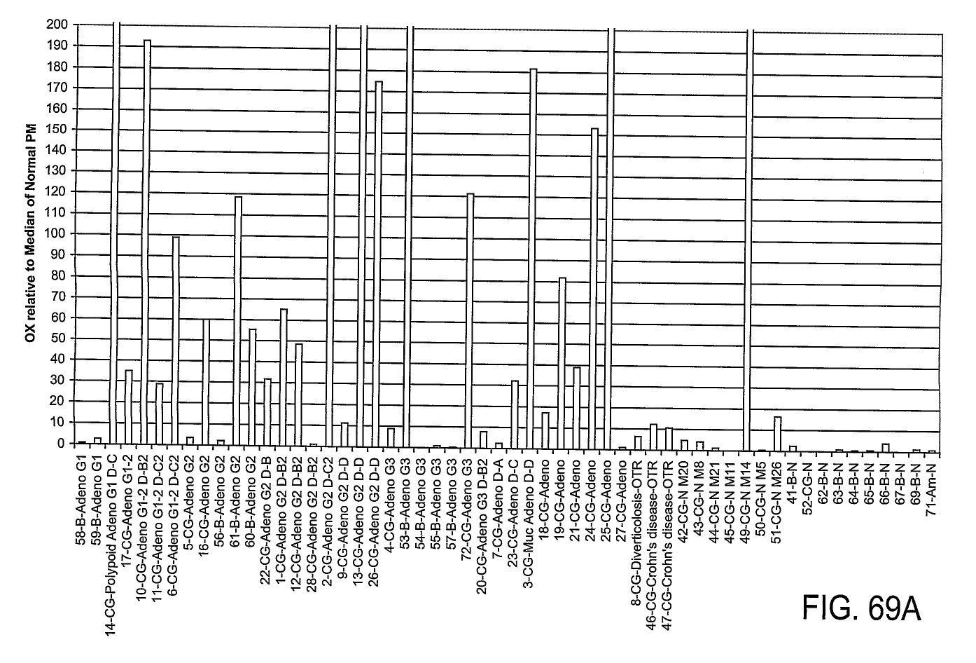

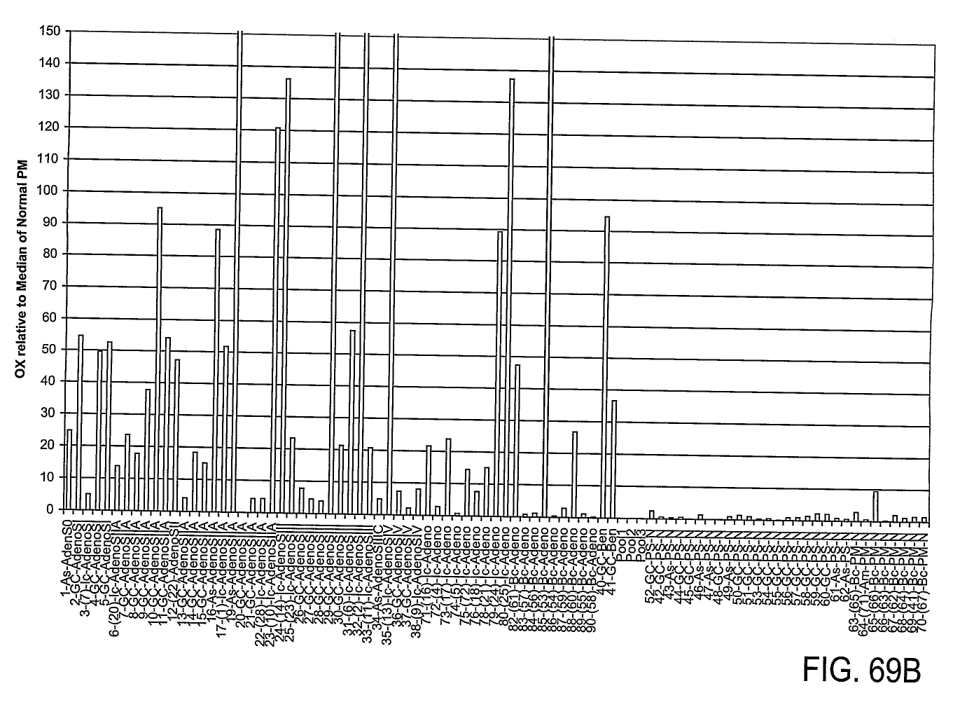

FIG. 69A is a histogram showing over expression of the Homo sapiens LEM domain containing 1 (LEMD1) transcripts in cancerous Colon samples relative to the normal samples.

As is evident from FIG. 69A, the expression of Homo sapiens LEM domain containing 1 (LEMD1) transcripts detectable by the above amplicon in cancer samples was significantly higher than in the non-cancerous samples (sample numbers 41, 63, 64, 65, 66, 69 and 71, Table 1_{—}4 above: “Tissue samples in colon cancer testing panel”). Notably an over-expression of at least 5 fold was found in 26 out of 36 adenocarcinoma samples.

Statistical analysis was applied to verify the significance of these results, as described below.

The P value for the difference in the expression levels of Homo sapiens LEM domain containing 1 (LEMD1) transcripts detectable by the above amplicon in Colon cancer samples versus the normal tissue samples was determined by T test as 8.52e-003.

Threshold of 5 fold over expression was found to differentiate between cancer and normal samples with P value of 6.04e-004 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T58132_seg21F (SEQ ID NO: 277) forward primer; and T58132_seg21R (SEQ ID NO: 278) reverse primer.

The present invention also preferably encompasses any amplicon obtained through the use of any suitable primer pair; for example, for the above experiment, the following amplicon was obtained as a non-limiting illustrative example only of a suitable amplicon: T58132_seg21 (SEQ ID NO: 279).

Expression of Homo sapiens LEM domain containing 1 (LEMD1) transcripts detectable by or according to seg21WT—T58132_seg21WT (SEQ ID NO: 279) amplicon and primers T58132_seg21WTF (SEQ ID NO: 277) and T58132_seg21WTR (SEQ ID NO: 278) was further measured by real time PCR using enlarged panel as shown in Table 1_{—}8. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), and G6PD (GenBank Accession No. NM_{—}000402 (SEQ ID NO:339); G6PD amplicon (SEQ ID NO: 311)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 52, 42, 43, 44, 45, 46, 47, 48, 49, 50, 51, 53, 54, 55, 56, 57, 58, 59, 60, 61, 62, 63, 64, 65, 66, 67, 68, 69 and 70, Table 1_{—}8 above), to obtain a value of fold up-regulation for each sample relative to the median of the normal samples.

FIG. 69B is a histogram showing over expression of the Homo sapiens LEM domain containing 1 (LEMD1) transcripts in cancerous Colon samples relative to the normal samples.

As is evident from FIG. 69B, the expression of Homo sapiens LEM domain containing 1 (LEMD1) transcripts detectable by the above amplicon in cancer samples was significantly higher than in the non-cancerous samples (sample numbers 52, 42, 43, 44, 45, 46, 47, 48, 49, 50, 51, 53, 54, 55, 56, 57, 58, 59, 60, 61, 62, 63, 64, 65, 66, 67, 68, 69 and 70, Table 1_{—}8 above). Notably an over-expression of at least 5 fold was found in 38 out of 55 adenocarcinoma samples.

Statistical analysis was applied to verify the significance of these results, as described below.

The P value for the difference in the expression levels of Homo sapiens LEM domain containing 1 (LEMD1) transcripts detectable by the above amplicon in Colon cancer samples versus the normal tissue samples was determined by T test as 1.20e-02.

Threshold of 5 fold over expression was found to differentiate between cancer and normal samples with P value of 1.48e-09 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T58132_seg21WTF (SEQ ID NO: 277) forward primer; and T58132_seg21WTR (SEQ ID NO: 278) reverse primer.

The present invention also preferably encompasses any amplicon obtained through the use of any suitable primer pair; for example, for the above experiment, the following amplicon was obtained as a non-limiting illustrative example only of a suitable amplicon: T58132_seg21WT (SEQ ID NO: 279).

Expression of Homo sapiens LEM Domain Containing 1 (LEMD1) T58132 Transcripts which are Detectable by Amplicon as Depicted in Sequence Name T58132_seg6F2R2 (SEQ ID NO: 273) in Normal and Cancerous Lung Tissues

Expression of Homo sapiens LEM domain containing 1 (LEMD1) transcripts detectable by or according to seg6F2R2—T58132_seg6F2R2 (SEQ ID NO: 273) amplicon and primers T58132_seg6F2 (SEQ ID NO: 271) and T58132_seg6R2 (SEQ ID NO: 272) was measured by real time PCR. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 47, 48, 49, 50, 90, 91, 92, 93, 96, 97, 98 and 99, Table 1_{—}2 above), to obtain a value of fold up-regulation for each sample relative to the median of the normal PM samples.

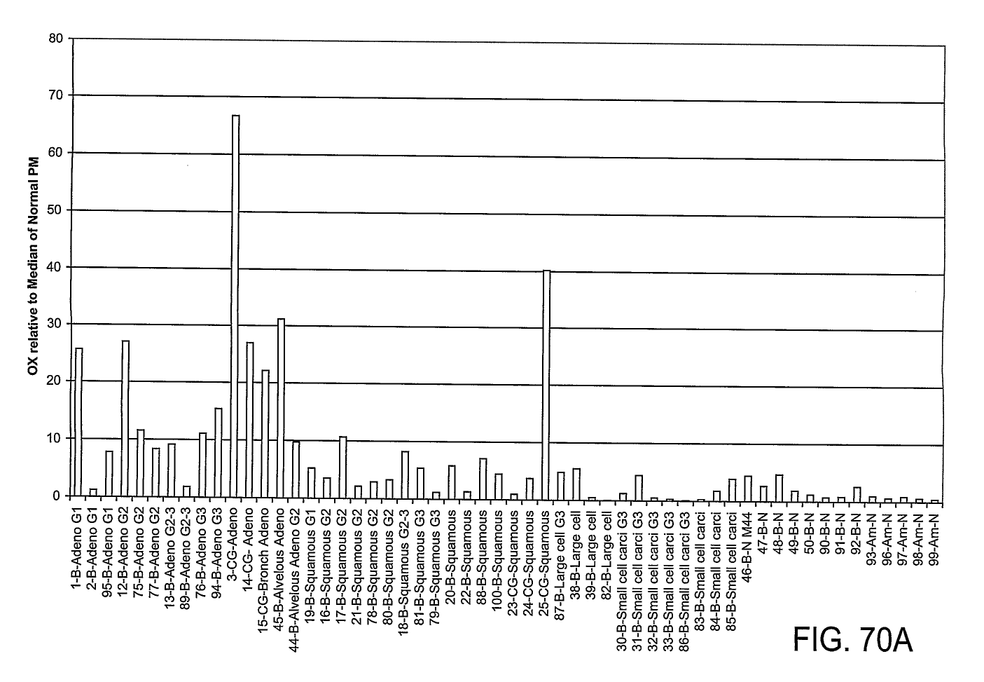

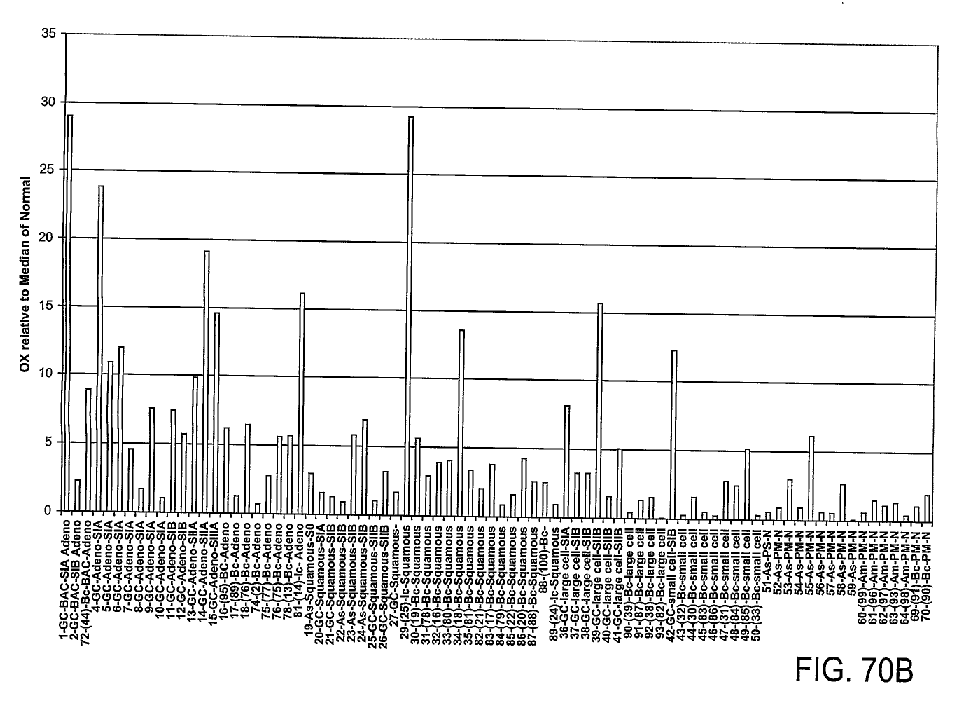

FIG. 70A is a histogram showing over expression of the Homo sapiens LEM domain containing 1 (LEMD1) transcripts in cancerous Lung samples relative to the normal samples.

As is evident from FIG. 70A, the expression of Homo sapiens LEM domain containing 1 (LEMD1) transcripts detectable by the above amplicon in non-small cell carcinoma samples, specifically, adenocarcinoma and squamous cell carcinoma, was significantly higher than in the non-cancerous samples (sample numbers 47, 48, 49, 50, 90, 91, 92, 93, 96, 97, 98 and 99, Table 1_{—}2 above). Notably an over-expression of at least 5 fold was found in 21 out of 35 non-small cell carcinoma samples, specifically—13 out of 15 adenocarcinoma samples and in 7 out of 16 squamous cell carcinoma samples.

Statistical analysis was applied to verify the significance of these results, as described below.

The P value for the difference in the expression levels of Homo sapiens LEM domain containing 1 (LEMD1) transcripts detectable by the above amplicon in Lung adenocarcinoma samples versus the normal tissue samples was determined by T test as 1.36e-003. The P value for the difference in the expression levels of Homo sapiens LEM domain containing 1 (LEMD1) transcripts detectable by the above amplicon in Lung squamous cell carcinoma samples versus the normal tissue samples was determined by T test as 4.68e-002. The P value for the difference in the expression levels of Homo sapiens LEM domain containing 1 (LEMD1) transcripts detectable by the above amplicon in Lung non-small cell carcinoma samples versus the normal tissue samples was determined by T test as 2.28e-004.

Threshold of 5 fold over expression was found to differentiate between adenocarcinoma and normal samples with P value of 5.23e-006 as checked by exact Fisher test. Threshold of 5 fold over expression was found to differentiate between squamous cell carcinoma and normal samples with P value of 9.66e-003 as checked by exact Fisher test. Threshold of 5 fold over expression was found to differentiate between non-small cell carcinoma and normal samples with P value of 1.85e-004 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T58132_seg6F2 (SEQ ID NO: 271) forward primer; and T58132_seg6R2 (SEQ ID NO: 272) reverse primer.

The present invention also preferably encompasses any amplicon obtained through the use of any suitable primer pair; for example, for the above experiment, the following amplicon was obtained as a non-limiting illustrative example only of a suitable amplicon: T58132_seg6F2R2 (SEQ ID NO: 273).

Expression of LEM domain containing 1 (LEMD1) transcripts detectable by or according to seg6F2R2-T58132_seg6F2R2 (SEQ ID NO: 273) amplicon and primers T58132_seg6F2 (SEQ ID NO: 271) and T58132_seg6R2 (SEQ ID NO: 272) was further measured by real time PCR using enlarged panel as shown in Table 1_{—}6. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 51, 52, 53, 54, 55, 56, 57, 58, 59, 60, 61, 62, 63, 64, 69 and 70, Table 1_{—}6 above), to obtain a value of fold up-regulation for each sample relative to the median of the normal samples.

FIG. 70B is a histogram showing over expression of the above-indicated LEM domain containing 1 (LEMD1) transcripts in cancerous Lung samples relative to the normal samples.

As is evident from FIG. 70B, the expression of LEM domain containing 1 (LEMD1) transcripts detectable by the above amplicon non-small cell carcinoma samples was significantly higher than in the non-cancerous samples (sample numbers 51, 52, 53, 54, 55, 56, 57, 58, 59, 60, 61, 62, 63, 64, 69 and 70, Table 1_{—}6 above).

Notably an over-expression of at least 5 fold was found in 24 out of 57 non-small cell carcinoma samples, specifically in 16 out of 23 adenocarcinoma samples, in 5 out of 24 squamous cell carcinoma samples, and in 3 out of 10 large cell carcinoma samples. Expression was also higher in 2 out of 9 small cell carcinoma samples than in the non-cancerous samples.

Statistical analysis was applied to verify the significance of these results, as described below.

The P value for the difference in the expression levels of LEM domain containing 1 (LEMD1) transcripts detectable by the above amplicon in Lung non-small cell carcinoma samples versus the normal tissue samples was determined by T test as 6.80e-006. The P value for the difference in the expression levels of LEM domain containing 1 (LEMD1) transcripts detectable by the above amplicon in Lung adenocarcinoma samples versus the normal tissue samples was determined by T test as 1.04e-004. The P value for the difference in the expression levels of LEM domain containing 1 (LEMD1) transcripts detectable by the above amplicon in Lung squamous cell carcinoma samples versus the normal tissue samples was determined by T test as 2.63e-002.

Threshold of 5 fold over expression was found to differentiate between non-small cell carcinoma and normal samples with P value of 5.60e-003 as checked by exact Fisher test. Threshold of 5 fold over expression was found to differentiate between adenocarcinoma and normal samples with P value of 7.86e-005 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T58132_seg6F2 (SEQ ID NO: 271) forward primer; and T58132_seg6R2 (SEQ ID NO: 272) reverse primer.

The present invention also preferably encompasses any amplicon obtained through the use of any suitable primer pair; for example, for the above experiment, the following amplicon was obtained as a non-limiting illustrative example only of a suitable amplicon: T58132_seg6F2R2 (SEQ ID NO: 273).

Expression of Homo sapiens LEM Domain Containing 1 (LEMD1) T58132 Transcripts which are Detectable by Amplicon as Depicted in Sequence Name T58132_seg6F2R2 (SEQ ID NO: 273) in Normal and Cancerous Ovary Tissues

Expression of Homo sapiens LEM domain containing 1 (LEMD1) transcripts detectable by or according to seg6F2R2—T58132_seg6F2R2 (SEQ ID NO: 273) amplicon and primers T58132_seg6F2 (SEQ ID NO: 271) and T58132_seg6R2 (SEQ ID NO: 272) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon —HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and GAPDH (GenBank Accession No. BC026907 (SEQ ID NO:338); GAPDH amplicon (SEQ ID NO: 308)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 45, 46, 71 and 48, Table 1_{—}1 above), to obtain a value of fold up-regulation for each sample relative to the median of the normal PM samples.

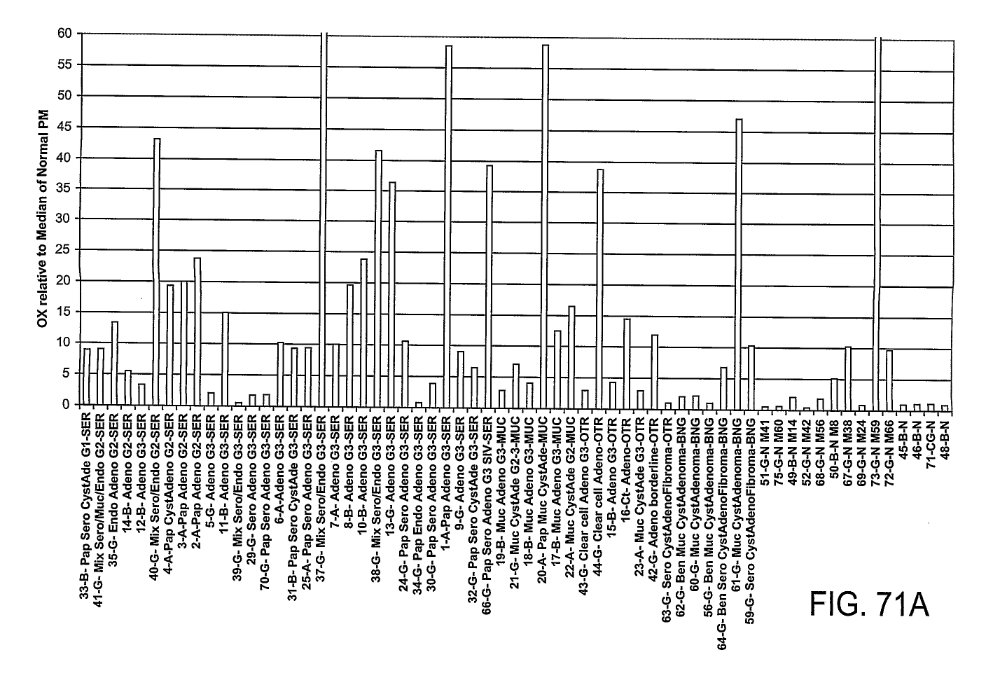

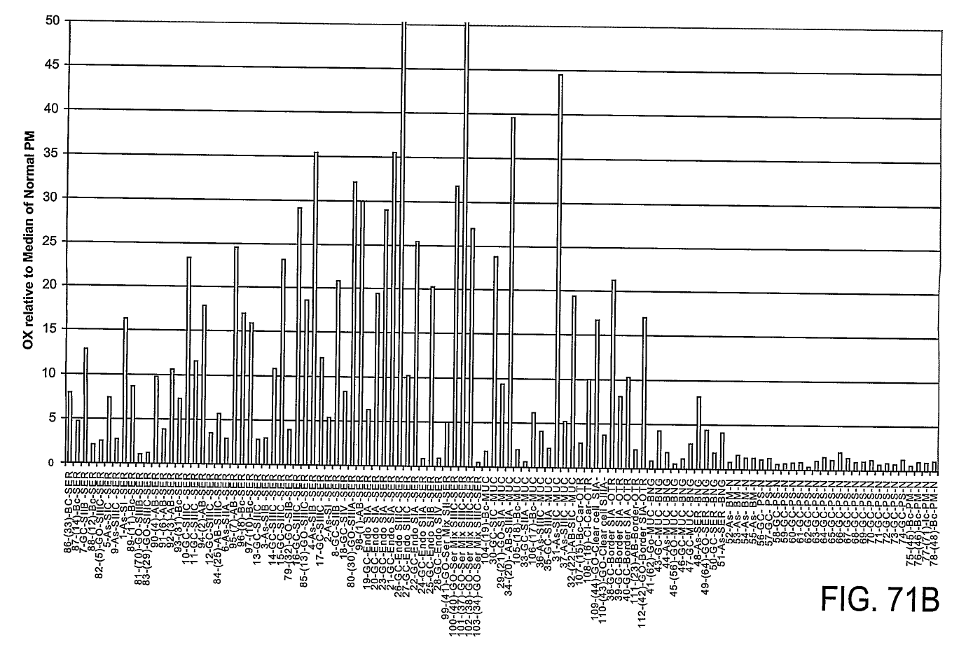

FIG. 71A is a histogram showing over expression of the Homo sapiens LEM domain containing 1 (LEMD1) transcripts in cancerous Ovary samples relative to the normal samples.

As is evident from FIG. 71A, the expression of Homo sapiens LEM domain containing 1 (LEMD1) transcripts detectable by the above amplicon in adenocarcinoma samples was significantly higher than in the non-cancerous samples (sample numbers 45, 46, 71 and 48, Table 1_{—}1 above). Notably an over-expression of at least 5 fold was found in 30 out of 43 adenocarcinoma samples, specifically in 23 out of 30 serous carcinoma samples and in 4 out of 6 mucinous carcinoma samples.

Statistical analysis was applied to verify the significance of these results, as described below.

The P value for the difference in the expression levels of Homo sapiens LEM domain containing 1 (LEMD1) transcripts detectable by the above amplicon in Ovary serous carcinoma samples versus the normal tissue samples was determined by T test as 1.04e-002. The P value for the difference in the expression levels of Homo sapiens LEM domain containing 1 (LEMI1) transcripts detectable by the above amplicon in Ovary adenocarcinoma samples versus the normal tissue samples was determined by T test as 1.94e-003.

Threshold of 5 fold over expression was found to differentiate between serous carcinoma and normal samples with P value of 7.12e-003 as checked by exact Fisher test. Threshold of 5 fold over expression was found to differentiate between all adenocarcinoma and normal samples with P value of 1.33e-002 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T58132_seg6F2 (SEQ ID NO: 271) forward primer; and T58132_seg6R2 (SEQ ID NO: 272) reverse primer.

The present invention also preferably encompasses any amplicon obtained through the use of any suitable primer pair; for example, for the above experiment, the following amplicon was obtained as a non-limiting illustrative example only of a suitable amplicon: T58132_seg6F2R2 (SEQ ID NO: 273).

Expression of Homo sapiens LEM domain containing 1 (LEMD1) transcripts detectable by or according to seg6F2R2—T58132_seg6F2R2 (SEQ ID NO: 273) amplicon and primers T58132_seg6F2 (SEQ ID NO: 271) and T58132_seg6R (SEQ ID NO: 272) was further measured by real time PCR using enlarged panel as shown in Table 1_{—}5. In parallel the expression of four housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)), and PBGD (GenBank Accession No. BC019323 (SEQ ID NO:336); amplicon—PBGD-amplicon (SEQ ID NO: 302)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 52-78, Table 1_{—}5 above), to obtain a value of fold up-regulation for each sample relative to the median of the normal samples.

FIG. 71B is a histogram showing over expression of the above-indicated Homo sapiens LEM domain containing 1 (LEMD1) transcripts in cancerous Ovary samples relative to the normal samples.

As is evident from FIG. 71B, the expression of Homo sapiens LEM domain containing 1 (LEMD1) transcripts detectable by the above amplicon in adenocarcinoma samples was significantly higher than in the non-cancerous samples (sample numbers 41, 43-78, Table 1_{—}5 above). In samples from serous adenocarcinoma correlation between over expression level and patient's age could be observed—over expression was higher as the patients were older. Notably an over-expression of at least 5 fold was found in 51 out of 74 adenocarcinoma samples, specifically in 26 out of 38 serous carcinoma samples, in 8 out of 10 endometroid-type adenocarcinoma samples, and in 7 out of 12 mucinous carcinoma samples.

Statistical analysis was applied to verify the significance of these results, as described below.

The P value for the difference in the expression levels of Homo sapiens LEM domain containing 1 (LEMD1) transcripts detectable by the above amplicon in Ovary adenocarcinoma samples versus the non cancerous tissue samples was determined by T test as 7.73e-08. Threshold of 5 fold over expression was found to differentiate between adeonocarcinoma and non-cancerous samples with P value of 1.96e-12 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T58132_seg6F2 (SEQ ID NO: 271) forward primer; and T58132_seg6R2 (SEQ ID NO: 272) reverse primer.

The present invention also preferably encompasses any amplicon obtained through the use of any suitable primer pair; for example, for the above experiment, the following amplicon was obtained as a non-limiting illustrative example only of a suitable amplicon: T58132_seg6F2R2 (SEQ ID NO: 273).

Expression of Homo sapiens LEM Domain Containing 1 (LEMD1) T58132 Transcripts which are Detectable by Amplicon as Depicted in Sequence Name T58132_seg8F2R2 (SEQ ID NO: 276) in Normal and Cancerous Lung Tissues

Expression of Homo sapiens LEM domain containing 1 (LEMD1) transcripts detectable by or according to seg8F2R2—T58132_seg8F2R2 (SEQ ID NO: 276) amplicon and primers T58132_seg8F2 (SEQ ID NO: 274) and T58132_seg8R2 (SEQ ID NO: 275) was measured by real time PCR. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 47, 48, 49, 50, 90, 91, 92, 93, 96, 97, 98 and 99, Table 1_{—}2 above), to obtain a value of fold up-regulation for each sample relative to the median of the normal PM samples.

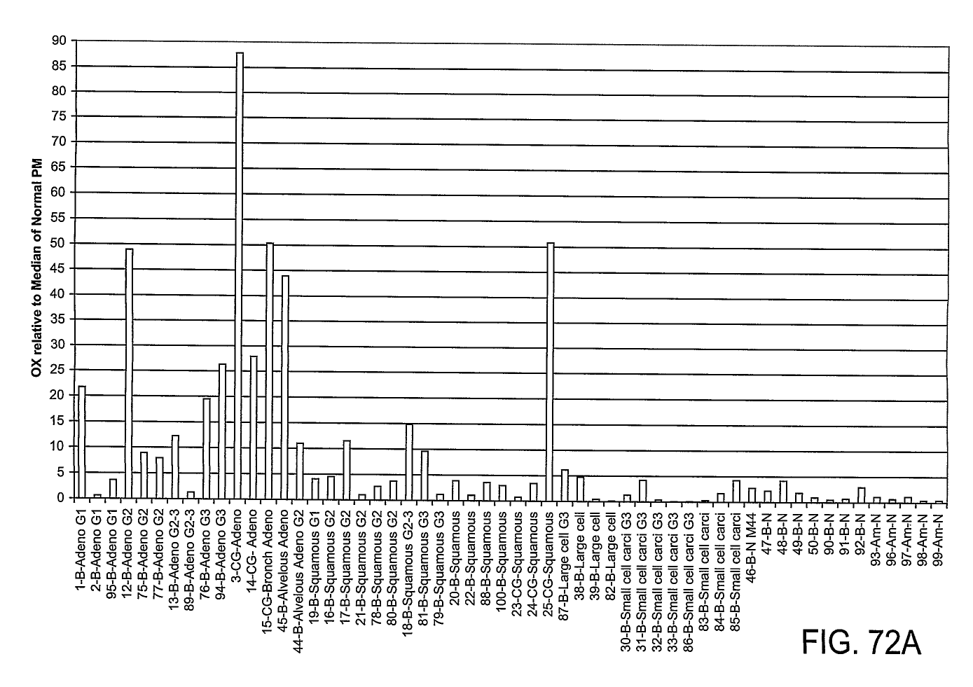

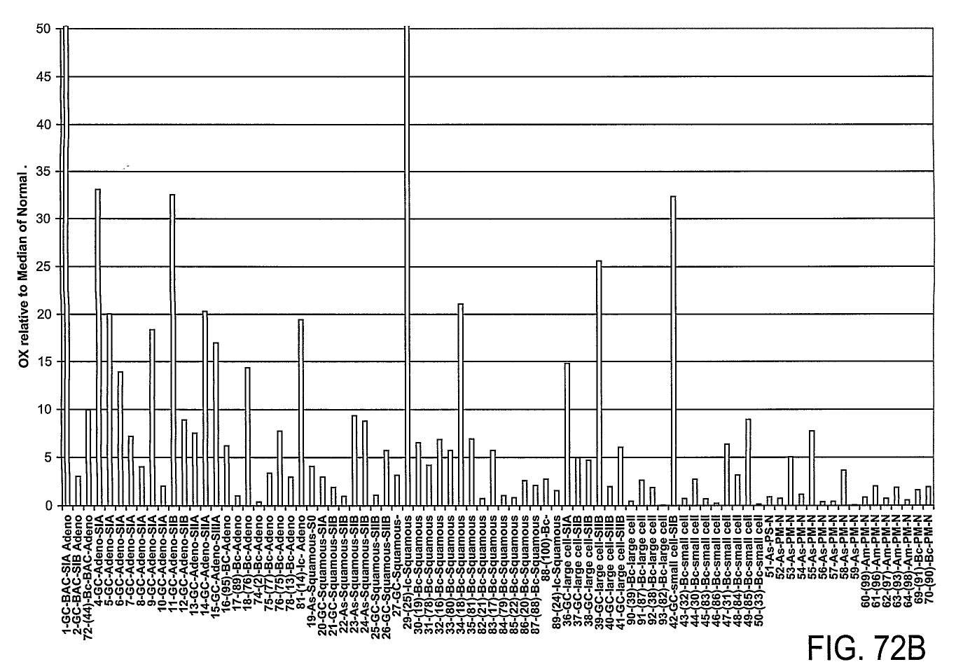

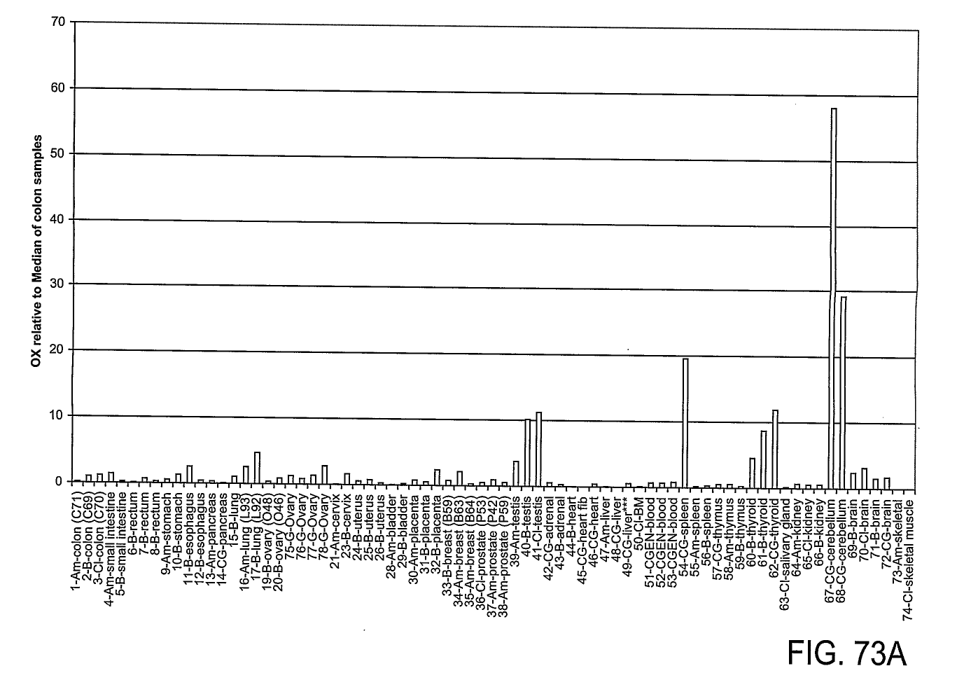

FIG. 72A is a histogram showing over expression of the Homo sapiens LEM domain containing 1 (LEMD1) transcripts in cancerous Lung samples relative to the normal samples.

As is evident from FIG. 72A, the expression of Homo sapiens LEM domain containing 1 (LEMD1) transcripts detectable by the above amplicon in adenocarcinoma was significantly higher than in the non-cancerous samples (sample numbers 47, 48, 49, 50, 90, 91, 92, 93, 96, 97, 98 and 99, Table 1_{—}2 above) and was higher in a few squamous cell carcinoma samples than in the non-cancerous samples. Notably an over-expression of at least 5 fold was in 17 out of 35 non-small cell carcinoma samples, specifically in 12 out of 15 adenocarcinoma samples and in 4 out of 16 squamous cell carcinoma samples.

Statistical analysis was applied to verify the significance of these results, as described below.

The P value for the difference in the expression levels of Homo sapiens LEM domain containing 1 (LEMD1) transcripts detectable by the above amplicon in Lung adenocarcinoma samples versus the normal tissue samples was determined by T test as 2.08e-003. The P value for the difference in the expression levels of Homo sapiens LEM domain containing 1 (LEMD1) transcripts detectable by the above amplicon in Lung non-small cell carcinoma samples versus the normal tissue samples was determined by T test as 4.66e-004.

Threshold of 5 fold over expression was found to differentiate between adenocarcinoma and normal samples with P value of 2.62e-005 as checked by exact Fisher test. Threshold of 5 fold over expression was found to differentiate between all non-small cell carcinoma and normal samples with P value of 1.66e-003 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T58132_seg8F2 (SEQ ID NO: 274) forward primer; and T58132_seg8R2 (SEQ ID NO: 275) reverse primer.

The present invention also preferably encompasses any amplicon obtained through the use of any suitable primer pair; for example, for the above experiment, the following amplicon was obtained as a non-limiting illustrative example only of a suitable amplicon: T58132_seg8F2R2 (SEQ ID NO: 276)

Expression of LEM domain containing 1 (LEMD1) transcripts detectable by or according to seg8F2R2—T58132_seg8F2R2 (SEQ ID NO: 276) amplicon and primers T58132_seg8F2 (SEQ ID NO: 274) and T58132_seg8R2 (SEQ ID NO: 275) was further measured by real time PCR using enlarged panel as shown in Table 1_{—}6. In parallel the expression of four housekeeping genes—HPRT1 (GenBank Accession No. NM 000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 51, 52, 53, 54, 55, 56, 57, 58, 59, 60, 61, 62, 63, 64, 69 and 70, Table 1_{—}6 above), to obtain a value of fold up-regulation for each sample relative to the median of the normal samples.

FIG. 72B is a histogram showing over expression of the above-indicated LEM domain containing 1 (LEMD1) transcripts in cancerous Lung samples relative to the normal samples.

As is evident from FIG. 72B, the expression of LEM domain containing 1 (LEMD1) transcripts detectable by the above amplicon non-small cell carcinoma samples was significantly higher than in the non-cancerous samples (sample numbers 51, 52, 53, 54, 55, 56, 57, 58, 59, 60, 61, 62, 63, 64, 69 and 70, Table 1_{—}6 above). Notably an over-expression of at least 5 fold was found in 29 out of 57 non-small cell carcinoma samples, specifically in 16 out of 23 adenocarcinoma samples, in 10 out of 24 squamous cell carcinoma samples, in 3 out of 10 large cell carcinoma samples. Expression was also higher in 3 out of 9 small cell carcinoma samples.

Statistical analysis was applied to verify the significance of these results, as described below.

The P value for the difference in the expression levels of LEM domain containing 1 (LEMD1) transcripts detectable by the above amplicon in Lung non small cell carcinoma samples versus the normal tissue samples was determined by T test as 1.51e-005. The P value for the difference in the expression levels of LEM domain containing 1 (LEMD1) transcripts detectable by the above amplicon in adenocarcinoma samples versus the normal tissue samples was determined by T test as 2.24e-004. The P value for the difference in the expression levels of LEM domain containing 1 (LEMD1) transcripts detectable by the above amplicon in Lung squamous cell carcinoma samples versus the normal tissue samples was determined by T test as 4.27e-002.

Threshold of 5 fold over expression was found to differentiate between non-small cell carcinoma and normal samples with P value of 5.25e-003 as checked by exact Fisher test. Threshold of 5 fold over expression was found to differentiate between adenocarcinoma and normal samples with P value of 4.94e-004 as checked by exact Fisher test. Threshold of 5 fold over expression was found to differentiate between squamous cell carcinoma and normal samples with P value of 4.92e-002 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T58132_seg8F2 (SEQ ID NO: 274) forward primer; and T58132_seg8R2 (SEQ ID NO: 275) reverse primer.

The present invention also preferably encompasses any amplicon obtained through the use of any suitable primer pair; for example, for the above experiment, the following amplicon was obtained as a non-limiting illustrative example only of a suitable amplicon: T58132_seg8F2R2 (SEQ ID NO: 276).

Expression of Homo sapiens LEM Domain Containing 1 (LEMD1) T58132 Transcripts which are Detectable by Amplicon as Depicted in Sequence Name T58132_seg8F2R2 (SEQ ID NO: 276) in Different Normal Tissues

Expression of Homo sapiens LEM domain containing 1 (LEMD1) transcripts detectable by or according to seg8F2R2—T58132_seg8F2R2 (SEQ ID NO: 276) amplicon and primers T58132 seg8F2 (SEQ ID NO: 274) and T58132_seg8R2 (SEQ ID NO: 275) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the geometric mean of the quantities of the housekeeping genes. The normalized quantity of each RT sample was then divided by the median of the quantities of the colon samples (sample numbers 1, 2 and 3, Table 1_{—}9 above), to obtain a value of relative expression of each sample relative to the median of the colon samples.

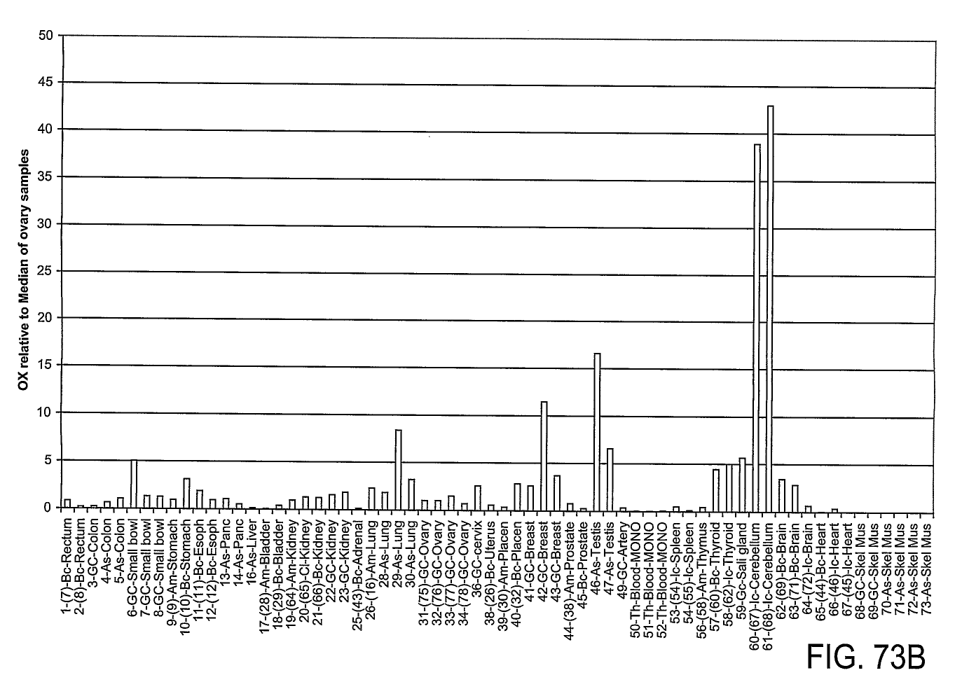

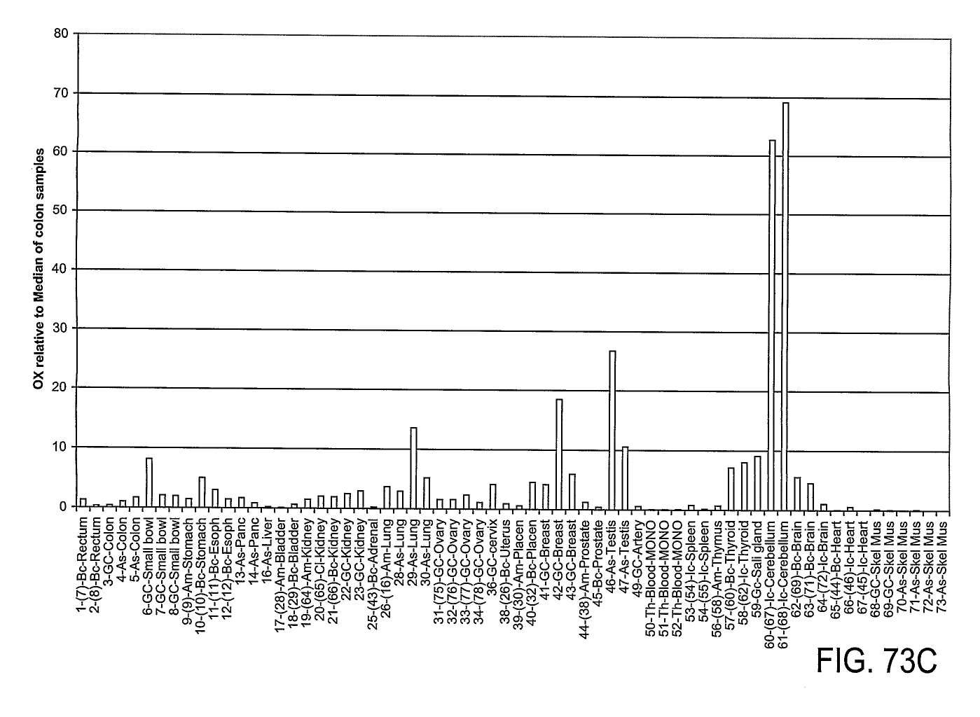

FIG. 73A is a histogram showing the expression of Homo sapiens LEM domain containing 1 (LEMD1) T58132 transcripts which are detectable by amplicon as depicted in sequence name T58132_seg8F2R2 (SEQ ID NO: 276) in different normal tissues.

Expression of Homo sapiens LEM domain containing 1 (LEMD1) transcripts detectable by or according to seg8F2R2—T58132_seg8F2R2 (SEQ ID NO: 276) amplicon and primers T58132_seg8F2 (SEQ ID NO: 274) and T58132_seg8R2 (SEQ ID NO: 275) was further measured by real time PCR using updated panel as shown in Table 1_{—}10. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), and TATA box (GenBank Accession No. NM_{—}003194 (SEQ ID NO:343); TATA amplicon (SEQ ID NO: 323)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the ovary samples (sample numbers 31, 32, 33 and 34, Table 1_{—}10 above), to obtain a value of relative expression of each sample relative to the median of the ovary samples as presented in FIG. 73B. The normalized quantity of each RT sample was also divided by the median of the quantities of the colon samples (sample numbers 3, 4, 5, Table 1_{—}10 above), to obtain a value of relative expression of each sample relative to the median of the colon samples as presented in FIG. 73C.

Expression of Homo sapiens LEM Domain Containing 1 (LEMD1) T58132 Transcripts which are Detectable by Amplicon as Depicted in Sequence Name T58132_seg8F2R2 (SEQ ID NO: 276) in Normal and Cancerous Ovary Tissues

Expression of Homo sapiens LEM domain containing 1 (LEMD1) transcripts detectable by or according to seg8F2R2—T58132_seg8F2R2 (SEQ ID NO: 276) amplicon and primers T58132_seg8F2 (SEQ ID NO: 274) and T58132_seg8R2 (SEQ ID NO: 275) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), HPRT1 (GenBank Accession No. NM 000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and GAPDH (GenBank Accession No. BC026907 (SEQ ID NO:338); GAPDH amplicon (SEQ ID NO: 308)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 45, 46, 71 and 48, Table 1_{—}1 above), to obtain a value of fold up-regulation for each sample relative to the median of the normal PM samples.

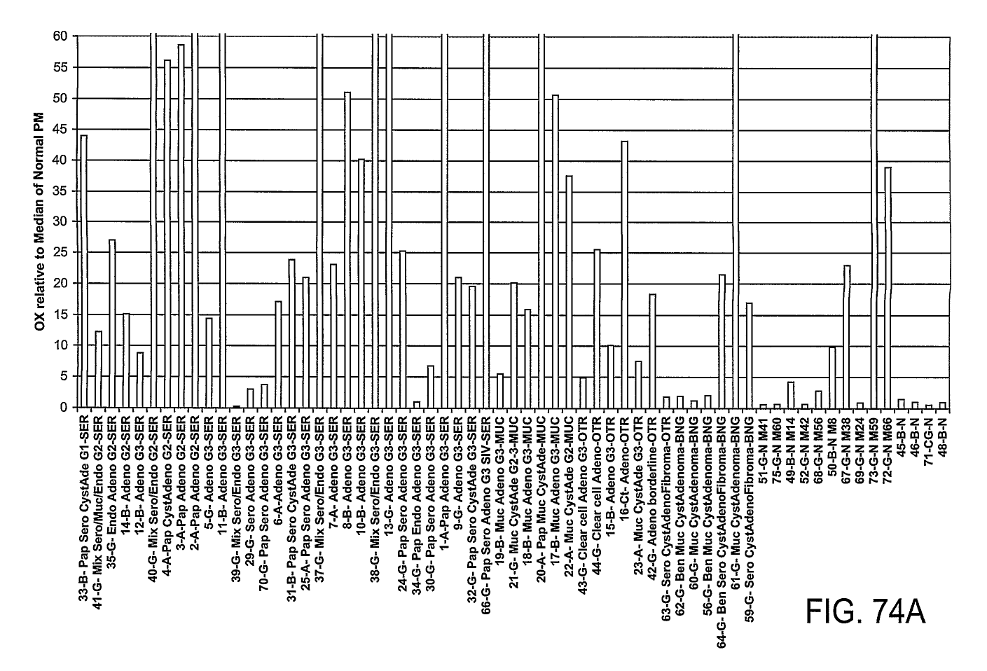

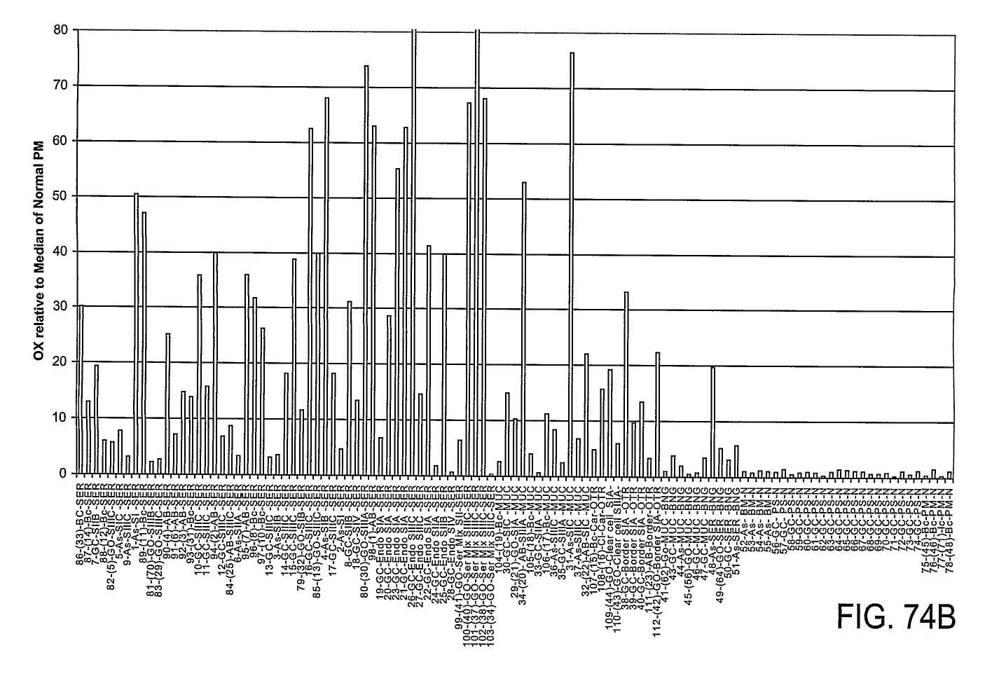

FIG. 74A is a histogram showing over expression of the Homo sapiens LEM domain containing 1 (LEMD1) transcripts in cancerous Ovary samples relative to the normal samples.

As is evident from FIG. 74A, the expression of Homo sapiens LEM domain containing 1 (LEMD1) transcripts detectable by the above amplicon in adenocarcinoma samples was significantly higher than in the non-cancerous samples (sample numbers 45, 46, 71 and 48, Table 1_{—}1 above). Notably an over-expression of at least 5 fold was found in 37 out of 43 adenocarcinoma samples, specifically in 26 out of 30 serous carcinoma samples and in 6 out of 6 mucinous carcinoma samples.

Statistical analysis was applied to verify the significance of these results, as described below.

The P value for the difference in the expression levels of Homo sapiens LEM domain containing 1 (LEMD1) transcripts detectable by the above amplicon in Ovary serous carcinoma samples versus the normal tissue samples was determined by T test as 1.24e-002. The P value for the difference in the expression levels of Homo sapiens LEM domain containing 1 (LEMD1) transcripts detectable by the above amplicon in all ovary adenocarcinoma samples versus the normal tissue samples was determined by T test as 4.14e-003.

Threshold of 5 fold over expression was found to differentiate between serous carcinoma and normal samples with P value of 1.51 e-003 as checked by exact Fisher test. Threshold of 5 fold over expression was found to differentiate between mucinous carcinoma and normal samples with P value of 4.76e-003 as checked by exact Fisher test. Threshold of 5 fold over expression was found to differentiate between all adenocarcinoma and normal samples with P value of 1.18e-003 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T58132_seg8F2 (SEQ ID NO: 274) forward primer; and T58132_seg8R2 (SEQ ID NO: 275) reverse primer.

The present invention also preferably encompasses any amplicon obtained through the use of any suitable primer pair; for example, for the above experiment, the following amplicon was obtained as a non-limiting illustrative example only of a suitable amplicon: T58132_seg8F2R2 (SEQ ID NO: 276).

Expression of Homo sapiens LEM domain containing 1 (LEMD1) transcripts detectable by or according to seg8F2R2—T58132_seg8F2R2 (SEQ ID NO: 276) amplicon and primers T58132_seg8F2 (SEQ ID NO: 274) and T58132_seg8R2 (SEQ ID NO: 275) was further measured by real time PCR using enlarged panel as shown in Table 1_{—}5. In parallel the expression of four housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)), and PBGD (GenBank Accession No. BC019323 (SEQ ID NO:336); amplicon—PBGD-amplicon (SEQ ID NO: 302)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 52-78, Table 1_{—}5 above), to obtain a value of fold up-regulation for each sample relative to the median of the normal samples.

FIG. 74B is a histogram showing over expression of the above-indicated Homo sapiens LEM domain containing 1 (LEMD1) transcripts in cancerous Ovary samples relative to the normal samples.

As is evident from FIG. 74B, the expression of Homo sapiens LEM domain containing 1 (LEMD1) transcripts detectable by the above amplicon in adenocarcinoma samples was significantly higher than in the non-cancerous samples (sample numbers 41, 43, 44-78, Table 1_{—}5 above). In samples from serous adenocarcinoma correlation between over expression level and patient's age could be observed—over expression was higher as the patients were older. Notably an over-expression of at least 5 fold was found in 58 out of 74 adenocarcinoma samples, specifically in 31 out of 38 serous carcinoma samples, in 8 out of 10 endometroid-type adenocarcinoma samples, and in 8 out of 12 mucinous carcinoma samples.

Statistical analysis was applied to verify the significance of these results, as described below.

The P value for the difference in the expression levels of Homo sapiens LEM domain containing 1 (LEMD1) transcripts detectable by the above amplicon in Ovary adenocarcinoma samples versus the non cancerous tissue samples was determined by T test as 1.38e-04. Threshold of 5 fold over expression was found to differentiate between adeonocarcinoma and non-cancerous samples with P value of 4.68e-13 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T58132_seg8F2 (SEQ ID NO: 274) forward primer; and T58132_seg8R2 (SEQ ID NO: 275) reverse primer.

The present invention also preferably encompasses any amplicon obtained through the use of any suitable primer pair; for example, for the above experiment, the following amplicon was obtained as a non-limiting illustrative example only of a suitable amplicon: T58132_seg8F2R2 (SEQ ID NO: 276).

Expression of Homo sapiens LEM Domain Containing 1 (LEMD1) T58132 Transcripts which are Detectable by Amplicon as Depicted in Sequence Name T58132_seg21 (SEQ ID NO: 279) in Normal and Cancerous Lung Tissues

Expression of Homo sapiens LEM domain containing 1 (LEMD1) transcripts detectable by or according to seg21—T58132_seg21 (SEQ ID NO: 279) amplicon and primers T58132_seg21F (SEQ ID NO: 277) and T58132_seg21R (SEQ ID NO: 278) was measured by real time PCR. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 47, 48, 49, 50, 90, 91, 92, 93, 96, 97, 98 and 99, Table 1_{—}2 above), to obtain a value of fold up-regulation for each sample relative to the median of the normal PM samples.

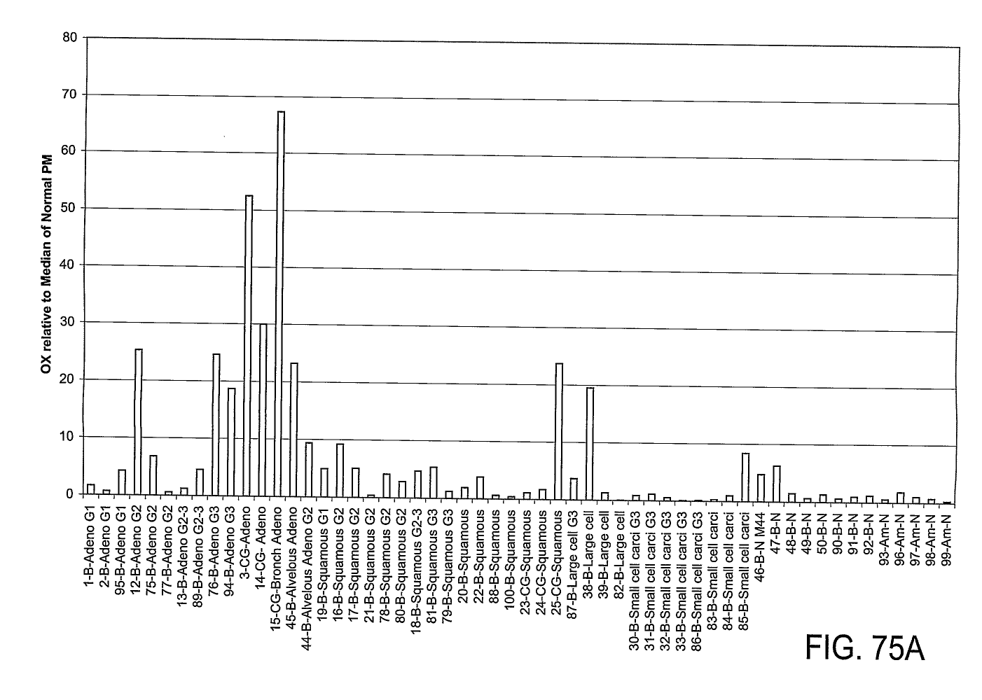

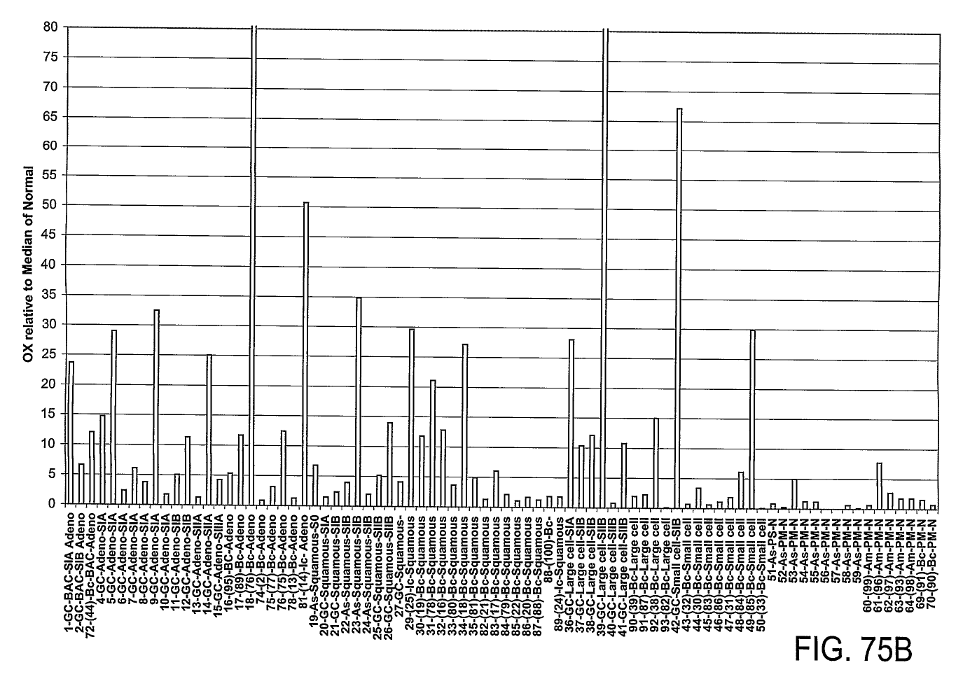

FIG. 75A is a histogram showing over expression of the Homo sapiens LEM domain containing 1 (LEMD1) transcripts in cancerous Lung samples relative to the normal samples.

As is evident from FIG. 75A, the expression of Homo sapiens LEM domain containing 1 (LEMD1) transcripts detectable by the above amplicon in adenocarcinoma was significantly higher than in the non-cancerous samples (sample numbers 47, 48, 49, 50, 90, 91, 92, 93, 96, 97, 98 and 99, Table 1_{—}2 above) and was higher in a few squamous cell carcinoma samples than in the non-cancerous samples. Notably an over-expression of at least 5 fold was found in 14 out of 35 non-small cell carcinoma samples, specifically in 9 out of 15 adenocarcinoma samples and in 4 out of 16 squamous cell carcinoma samples.

Statistical analysis was applied to verify the significance of these results, as described below.

The P value for the difference in the expression levels of Homo sapiens LEM domain containing 1 (LEMD1) transcripts detectable by the above amplicon in Lung adenocarcinoma samples versus the normal tissue samples was determined by T test as 6.20e-003. The P value for the difference in the expression levels of Homo sapiens LEM domain containing 1 (LEMD1) transcripts detectable by the above amplicon in all Lung non-small cell carcinoma samples versus the normal tissue samples was determined by T test as 1.34e-003.

Threshold of 5 fold over expression was found to differentiate between adenocarcinoma and normal samples with P value of 7.42e-003 as checked by exact Fisher test. Threshold of 5 fold over expression was found to differentiate between all non-small cell carcinoma and normal samples with P value of 4.12e-002 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T58132_seg21F (SEQ ID NO: 277) forward primer; and T58132_seg21R (SEQ ID NO: 278) reverse primer.

The present invention also preferably encompasses any amplicon obtained through the use of any suitable primer pair; for example, for the above experiment, the following amplicon was obtained as a non-limiting illustrative example only of a suitable amplicon: T58132_seg21 (SEQ ID NO: 279).

Expression of LEM domain containing 1 (LEMD1) transcripts detectable by or according to seg21WT—T58132_seg21WT (SEQ ID NO: 279) amplicon and primers T58132_seg21WTF (SEQ ID NO: 277) and T58132_seg21WTR (SEQ ID NO: 278) was further measured by real time PCR using enlarged panel as shown in Table 1_{—}6. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 51, 52, 53, 54, 55, 56, 57, 58, 59, 60, 61, 62, 63, 64, 69 and 70, Table 1_{—}6 above), to obtain a value of fold up-regulation for each sample relative to the median of the normal samples.

FIG. 75B is a histogram showing over expression of the above-indicated LEM domain containing 1 (LEMD1) transcripts in cancerous Lung samples relative to the normal samples.

As is evident from FIG. 75B, the expression of LEM domain containing 1 (LEMD1) transcripts detectable by the above amplicon in non-small cell carcinoma samples was significantly higher than in the non-cancerous samples (sample numbers 51, 52, 53, 54, 55, 56, 57, 58, 59, 60, 61, 62, 63, 64, 69 and 70, Table 1_{—}6 above).

Notably an over-expression of at least 5 fold was found in 32 out of 58 non-small cell carcinoma samples, specifically in 16 out of 24 adenocarcinoma samples, in 10 out of 24 squamous cell carcinoma samples, in 6 out of 10 large cell carcinoma samples.

Expression was also higher in 3 out of 9 small cell carcinoma samples than in the non-cancerous samples.

Statistical analysis was applied to verify the significance of these results, as described below.

The P value for the difference in the expression levels of LEM domain containing 1 (LEMD1) transcripts detectable by the above amplicon in Lung non-small cell carcinoma samples versus the normal tissue samples was determined by T test as 3.47e-004. The P value for the difference in the expression levels of LEM domain containing 1 (LEMD1) transcripts detectable by the above amplicon in Lung adenocarcinoma samples versus the normal tissue samples was determined by T test as 2.10e-003. The P value for the difference in the expression levels of LEM domain containing 1 (LEMD1) transcripts detectable by the above amplicon in Lung squamous cell carcinoma samples versus the normal tissue samples was determined by T test as 3.14e-003.

Threshold of 5 fold over expression was found to differentiate between non-small cell carcinoma and normal samples with P value of 3.26e-004 as checked by exact Fisher test. Threshold of 5 fold over expression was found to differentiate between adenocarcinoma and normal samples with P value of 1.36e-004 as checked by exact Fisher test. Threshold of 5 fold over expression was found to differentiate between squamous cell carcinoma and normal samples with P value of 1.46e-002 as checked by exact Fisher test. Threshold of 5 fold over expression was found to differentiate between large cell carcinoma and normal samples with P value of 5.24e-003 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T58132_seg21WTF (SEQ ID NO: 277) forward primer; and T58132_seg21WTR (SEQ ID NO: 278) reverse primer.

The present invention also preferably encompasses any amplicon obtained through the use of any suitable primer pair; for example, for the above experiment, the following amplicon was obtained as a non-limiting illustrative example only of a suitable amplicon: T58132_seg21WT (SEQ ID NO: 279).

Expression of Homo sapiens LEM Domain Containing 1 (LEMD1) T58132 Transcripts which are Detectable by Amplicon as Depicted in Sequence Name T58132_seg21 (SEQ ID NO: 279) in Different Normal Tissues

Expression of Homo sapiens LEM domain containing 1 (LEMD1) transcripts detectable by or according to seg21—T58132_seg21 (SEQ ID NO: 279) amplicon and primers T58132_seg21F (SEQ ID NO: 277) and T58132_seg21R (SEQ ID NO: 278) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the geometric mean of the quantities of the housekeeping genes. The normalized quantity of each RT sample was then divided by the median of the quantities of the colon samples (sample numbers 1, 2 and 3, Table 1_{—}9 above), to obtain a value of relative expression of each sample relative to the median of the colon samples.

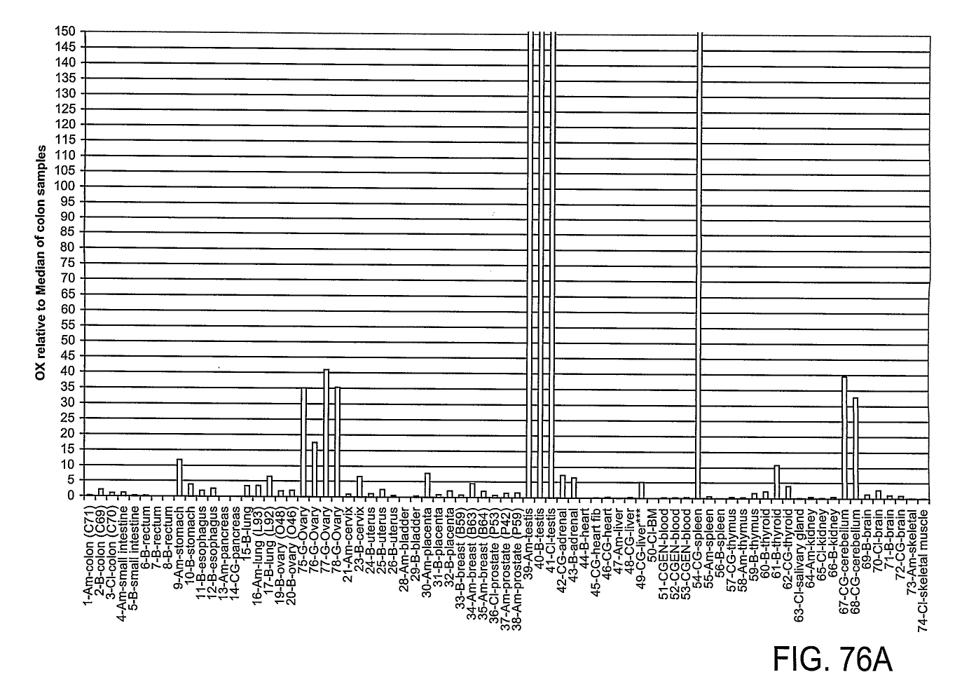

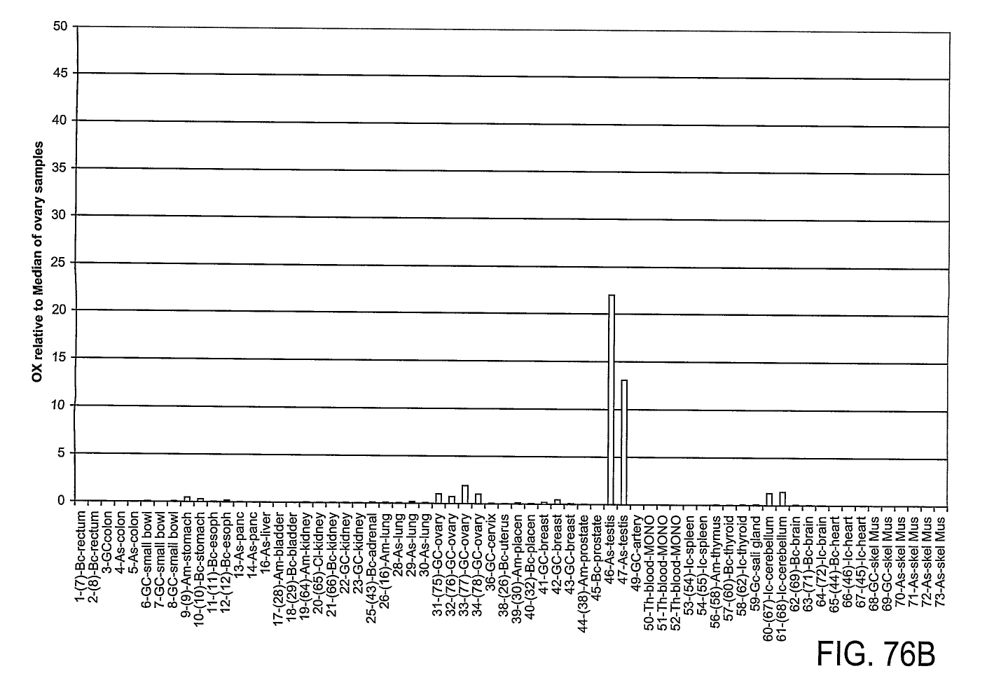

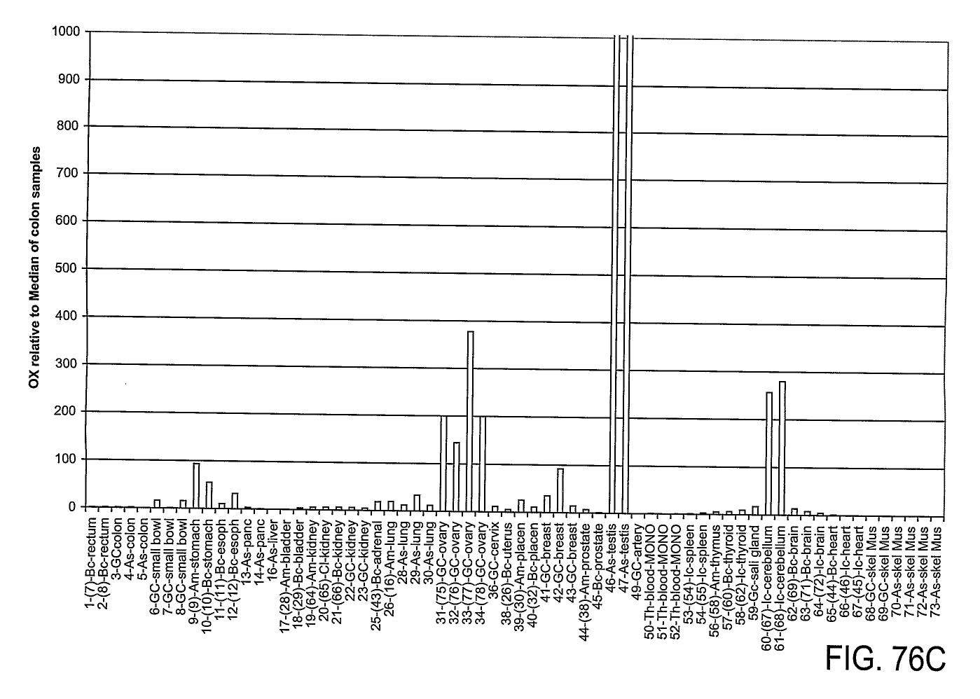

FIG. 76A is a histogram showing the expression of Homo sapiens LEM domain containing 1 (LEMD1) T58132 transcripts which are detectable by amplicon as depicted in sequence name T58132_seg21 (SEQ ID NO: 279) in different normal tissues.

Expression of Homo sapiens LEM domain containing 1 (LEMD1) transcripts detectable by or according to seg21WT-T58132_seg21WT (SEQ ID NO: 279) amplicon and primers T58132_seg21WTF (SEQ ID NO: 277) and T58132_seg21WTR (SEQ ID NO: 278) was further measured by real time PCR using updated panel as shown in Table 1_{—}10. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), and TATA box (GenBank Accession No. NM_{—}003194 (SEQ ID NO:343); TATA amplicon (SEQ ID NO: 323)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the ovary samples (sample numbers 31, 32, 33 and 34, Table 1_{—}9 above), to obtain a value of relative expression of each sample relative to the median of the ovary samples as presented in FIG. 76B. The normalized quantity of each RT sample was also divided by the median of the quantities of the colon samples (sample numbers 3, 4, 5, Table 1_{—}9 above), to obtain a value of relative expression of each sample relative to the median of the colon samples as presented in FIG. 76C.

Expression of Homo sapiens LEM Domain Containing 1 (LEMD1) T58132 Transcripts which are Detectable by Amplicon as Depicted in Sequence Name T58132_seg21 (SEQ ID NO: 279) in Normal and Cancerous Ovary Tissues

Expression of Homo sapiens LEM domain containing 1 (LEMD1) transcripts detectable by or according to seg21—T58132_seg21 (SEQ ID NO: 279) amplicon and primers T58132_seg21F (SEQ ID NO: 277) and T58132_seg21R (SEQ ID NO: 278) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and GAPDH (GenBank Accession No. BC026907 (SEQ ID NO:338); GAPDH amplicon (SEQ ID NO: 308)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 45, 46, 71 and 48, Table 1_{—}1 above), to obtain a value of fold up-regulation for each sample relative to the median of the normal PM samples.

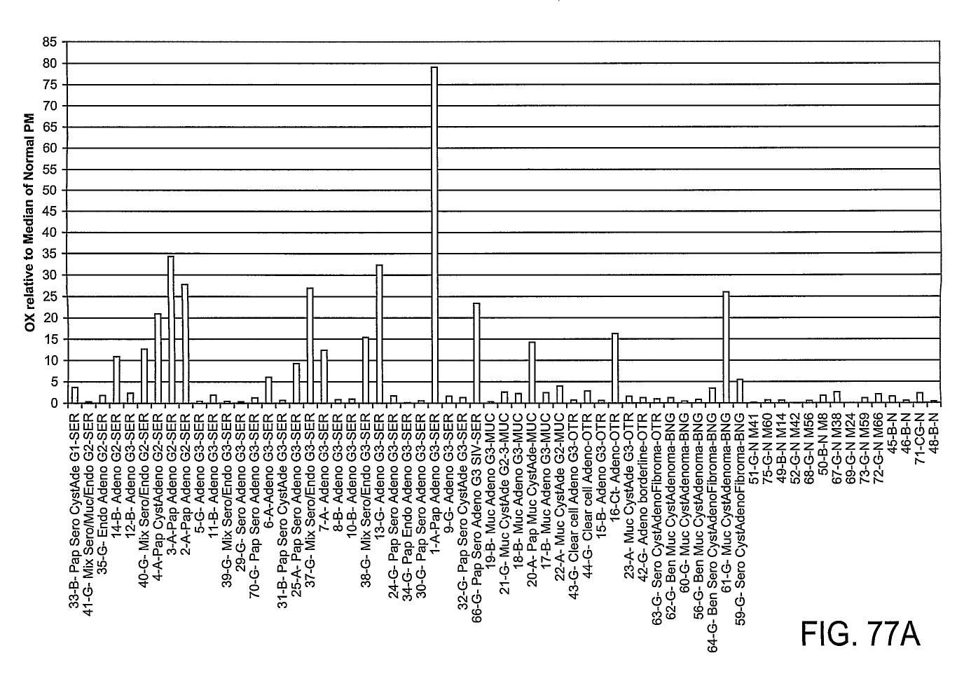

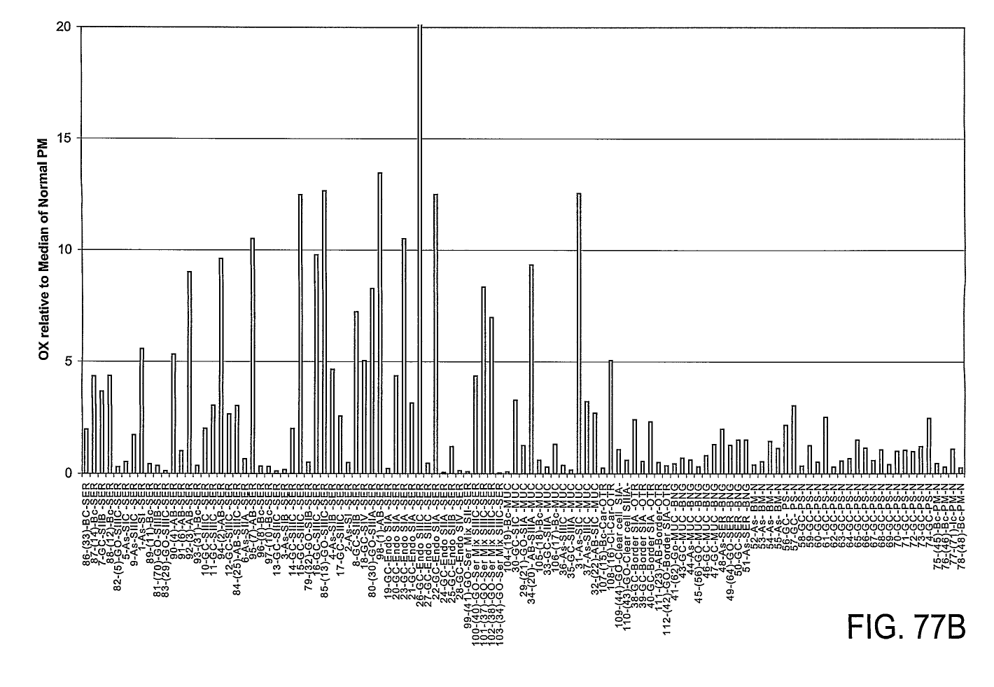

FIG. 77A is a histogram showing over expression of the Homo sapiens LEM domain containing 1 (LEMD1) transcripts in cancerous Ovary samples relative to the normal samples.

As is evident from FIG. 77A, the expression of Homo sapiens LEM domain containing 1 (LEMD1) transcripts detectable by the above amplicon adenocarcinoma samples was higher than in the non-cancerous samples (sample numbers 45, 46, 71 and 48, Table 1_{—}1 above). Notably an over-expression of at least 5 fold was found in 15 out of 43 adenocarcinoma samples.

Statistical analysis was applied to verify the significance of these results, as described below.

The P value for the difference in the expression levels of Homo sapiens LEM domain containing 1 (LEMD1) transcripts detectable by the above amplicon in Ovary serous carcinoma samples versus the normal tissue samples was determined by T test as 3.18e-003. The P value for the difference in the expression levels of Homo sapiens LEM domain containing 1 (LEMD1) transcripts detectable by the above amplicon in all ovary adenocarcinoma samples versus the normal tissue samples was determined by T test as 1.41e-003.

The above values demonstrate statistical significance of the results.

Primer pairs are also optionally and preferably encompassed within the present invention; for example, for the above experiment, the following primer pair was used as a non-limiting illustrative example only of a suitable primer pair: T58132_seg21F (SEQ ID NO: 277) forward primer; and T58132_seg21R (SEQ ID NO: 278) reverse primer.

The present invention also preferably encompasses any amplicon obtained through the use of any suitable primer pair; for example, for the above experiment, the following amplicon was obtained as a non-limiting illustrative example only of a suitable amplicon: T58132_seg21 (SEQ ID NO: 279).

Expression of Homo sapiens LEM domain containing 1 (LEMD1) transcripts detectable by or according to seg21WT—T58132_seg21WT (SEQ ID NO: 279) amplicon and primers T58132_seg21WTF (SEQ ID NO: 277) and T58132_seg21WTR (SEQ ID NO: 278) was further measured by real time PCR using enlarged panel as shown in Table 1_{—}5. In parallel the expression of four housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)), and PBGD (GenBank Accession No. BC019323 (SEQ ID NO:336); amplicon—PBGD-amplicon (SEQ ID NO: 302)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 52-78, Table 1_{—}5 above), to obtain a value of fold up-regulation for each sample relative to the median of the normal samples.

FIG. 77B is a histogram showing over expression of the above-indicated Homo sapiens LEM domain containing 1 (LEMD1) transcripts in cancerous Ovary samples relative to the normal samples.

As is evident from FIG. 77B, the expression of Homo sapiens LEM domain containing 1 (LEMD1) transcripts detectable by the above amplicon in adenocarcinoma samples was higher than in the non-cancerous samples (sample numbers 41, 43, 44-78, Table 1_{—}5 above). In samples from serous adenocarcinoma correlation between over expression level and patient's age could be observed-over expression was higher as the patients were older. Notably an over-expression of at least 5 fold was found in 20 out of 74 adenocarcinoma samples, specifically in 12 out of 38 serous carcinoma samples, in 3 out of 10 endometroid-type adenocarcinoma samples, and in 2 out of 12 mucinous carcinoma samples.

Statistical analysis was applied to verify the significance of these results, as described below.

The P value for the difference in the expression levels of Homo sapiens LEM domain containing 1 (LEMD1) transcripts detectable by the above amplicon in Ovary adenocarcinoma samples versus the non cancerous tissue samples was determined by T test as 8.20e-04. Threshold of 5 fold over expression was found to differentiate between adeonocarcinoma and non-cancerous samples with P value of 1.10e-04 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T58132_seg21WTF (SEQ ID NO: 277) forward primer; and T58132_seg21WTR (SEQ ID NO: 278) reverse primer.

The present invention also preferably encompasses any amplicon obtained through the use of any suitable primer pair; for example, for the above experiment, the following amplicon was obtained as a non-limiting illustrative example only of a suitable amplicon: T58132_seg21WT (SEQ ID NO: 279).

Expression of LEM Domain Containing 1 (LEMD1) T58132 Transcripts which are Detectable by Amplicon as Depicted in Sequence Name T58132_seg6F2R2 (SEQ ID NO: 273) in Normal and Cancerous Breast Tissues

Expression of LEM domain containing 1 (LEMD1) transcripts detectable by or according to seg6F2R2-T58132_seg6F2R2 (SEQ ID NO: 273) amplicon and primers T58132_seg6F2 (SEQ ID NO: 271) and T58132_seg6R2 (SEQ ID NO: 272) was measured by real time PCR. In parallel the expression of four housekeeping genes—PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)), RPL19 (GenBank Accession No. NM_{—}000981 (SEQ ID NO:342); RPL19 amplicon (SEQ ID NO: 320)) and G6PD (GenBank Accession No. NM_{—}000402 (SEQ ID NO:339); G6PD amplicon (SEQ ID NO: 311)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (Sample Nos. 43, 45, 46, 47, 48, 49, 50, 51, 52, 54, 56, 58, 59, 60, 61, 62, 63, 64, 65, 66, 67, 68 and 69, Table 1_{—}7, above), to obtain a value of fold differential expression for each sample relative to the median of the normal samples.

In one experiment that was carried out no differential expression in the cancerous samples relative to the normal samples was observed.

Primer pairs are also optionally and preferably encompassed within the present invention; for example, for the above experiment, the following primer pair was used as a non-limiting illustrative example only of a suitable primer pair: T58132_seg6F2 (SEQ ID NO: 271) forward primer; and T58132_seg6R2 (SEQ ID NO: 272) reverse primer.

The present invention also preferably encompasses any amplicon obtained through the use of any suitable primer pair; for example, for the above experiment, the following amplicon was obtained as a non-limiting illustrative example only of a suitable amplicon: T58132_seg6F2R2 (SEQ ID NO: 273).

### Description for Cluster T86235

Cluster T86235 (internal ID 15560128) features 10 transcript(s) and 12 segment(s) of interest, the names for which are given in Tables 150 and 151, respectively. The selected protein variants are given in table 152.

TABLE 150
Transcripts of interest
Transcript Name

T86235_T5 (SEQ ID NO: 440)
T86235_T6 (SEQ ID NO: 441)
T86235_T7 (SEQ ID NO: 442)
T86235_T11 (SEQ ID NO: 443)
T86235_T13 (SEQ ID NO: 444)
T86235_T15 (SEQ ID NO: 445)
T86235_T18 (SEQ ID NO: 446)
T86235_T26 (SEQ ID NO: 447)
T86235_T32 (SEQ ID NO: 448)
T86235_T36 (SEQ ID NO: 449)

TABLE 151
Segments of interest
Segment Name

T86235_N2 (SEQ ID NO: 450)
T86235_N19 (SEQ ID NO: 451)
T86235_N33 (SEQ ID NO: 452)
T86235_N39 (SEQ ID NO: 453)
T86235_N41 (SEQ ID NO: 454)
T86235_N53 (SEQ ID NO: 455)
T86235_N55 (SEQ ID NO: 456)
T86235_N12 (SEQ ID NO: 457)
T86235_N13 (SEQ ID NO: 458)
T86235_N14 (SEQ ID NO: 459)
T86235_N46 (SEQ ID NO: 460)
T86235_N47 (SEQ ID NO: 461)

TABLE 152
Proteins of interest

| Protein Name | Corresponding Transcript(s) |
|---|---|
| T86235_P4 (SEQ ID NO: 469) | T86235_T13 (SEQ ID NO: 444); |
| | T86235_T15 (SEQ ID NO: 445) |
| T86235_P5 (SEQ ID NO: 470) | T86235_T5 (SEQ ID NO: 440) |
| T86235_P6 (SEQ ID NO: 471) | T86235_T6 (SEQ ID NO: 441) |
| T86235_P7 (SEQ ID NO: 472) | T86235_T7 (SEQ ID NO: 442) |
| T86235_P11 (SEQ ID NO: 473) | T86235_T11 (SEQ ID NO: 443) |
| T86235_P15 (SEQ ID NO: 474) | T86235_T18 (SEQ ID NO: 446) |
| T86235_P21 (SEQ ID NO: 475) | T86235_T26 (SEQ ID NO: 447) |
| T86235_P26 (SEQ ID NO: 476) | T86235_T32 (SEQ ID NO: 448) |
| T86235_P30 (SEQ ID NO: 477) | T86235_T36 (SEQ ID NO: 449) |

These sequences are variants of the known protein Trophinin-associated protein (SEQ ID NO:462) (SwissProt accession identifier TAST_HUMAN (SEQ ID NO: 463); known also according to the synonyms Tastin; Trophinin-assisting protein), referred to herein as the previously known protein.

Protein Trophinin-associated protein (SEQ ID NO:462) is known or believed to have the following function(s): Could be involved with bystin and trophinin in a cell adhesion molecule complex that mediates an initial attachment of the blastocyst to uterine epithelial cells at the time of the embryo implantation. Protein Trophinin-associated protein (SEQ ID NO:462) localization is believed to be Cytoplasmic.

The following GO Annotation(s) apply to the previously known protein. The following annotation(s) were found: cell adhesion, which are annotation(s) related to Biological Process; and cytoplasm, which are annotation(s) related to Cellular Component.

PCT/IB2005/004037 and US Patent Application No. US 2006-0046257, filed on Jan. 27, 2005 and assigned to the applicant of the present invention, disclose protein sequence of T86235_P30 (SEQ ID NO:477) among other sequences of T86235 contig, and assays and methods of use thereof in the diagnosis of lung cancer. PCT/US05/03002 and U.S. patent application Ser. No. 11/051,646, filed on Jan. 27, 2005 and assigned to the applicant of the present invention, disclose polynucleotides of T86235 contig, and assays and methods of use thereof in the diagnosis using NAT based technologies. In this application the T86235 polynucleotides are described as being overexpressed in brain malignant tumors, epithelial malignant tumors, a mixture of malignant tumors from different tissues, skin malignancies and lung cancer.

In addition, the following proteins were previously disclosed by the inventors: T86235_P7, T86235_P11 and T86235_P26. T86235_P7 and T86235_P26 were disclosed in published PCT application no WO2004/096979, hereby incorporated by reference as if fully set forth herein. T86235_P11 was disclosed in published PCT application no WO2005/071058, hereby incorporated by reference as if fully set forth herein.

Unexpectedly, novel T86235 variant polynucleotides and their respective encoded polypeptides were identified and are now provided. The novel T86235 variants are now disclosed to be differentially expressed in breast cancer, ovarian cancer and lung cancer. Furthermore, the following variants have also been shown to have surprising new diagnostic utilities as they are now disclosed to be differentially expressed in breast cancer, ovarian cancer and lung cancer: T86235_P7 and T86235_P26. Surprisingly, T86235_P30 (SEQ ID NO:477) polypeptide and the known wild type T86235 polynucleotides and their respective encoded polypeptides are now disclosed to be differentially expressed in breast cancer and ovarian cancer, and therefore may be contemplated within the scope of the present invention as diagnostic markers for these diseases.

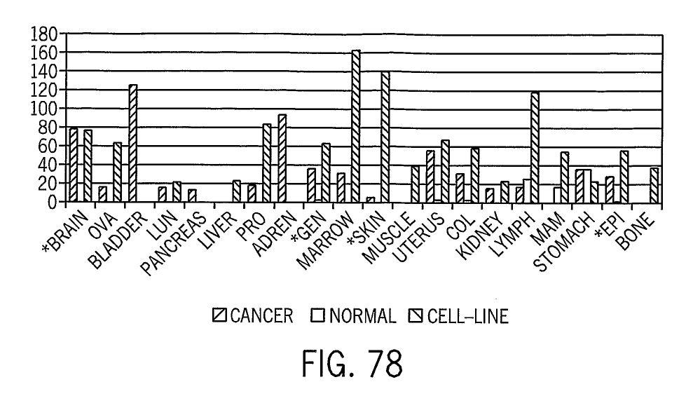

Cluster T86235 can be used as a diagnostic marker according to overexpression of transcripts of this cluster in cancer. Expression of such transcripts in normal tissues is also given according to the previously described methods. The term “number” in the left hand column of the table and the numbers on the y-axis of the FIG. 78 refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 78 and Table 153. This cluster is overexpressed (at least at a minimum level) in the following pathological conditions: brain malignant tumors, a mixture of malignant tumors from different tissues, skin malignancies and epithelial malignant tumors.

TABLE 153
Normal tissue distribution

| Name of Tissue | Number |
|---|---|
| brain | 0 |
| ovary | 0 |
| bladder | 0 |
| lung | 1 |
| pancreas | 0 |
| liver | 0 |
| prostate | 0 |
| adrenal | 0 |
| general | 3 |
| bone marrow | 0 |
| skin | 0 |
| muscle | 1 |
| uterus | 4 |
| colon | 3 |
| kidney | 0 |
| lymph nodes | 26 |
| breast | 17 |
| stomach | 36 |
| epithelial | 2 |
| bone | 0 |

TABLE 154
P values and ratios for expression in cancerous tissue

| Name of Tissue | P1 | P2 | SP1 | R3 | SP2 | R4 |
|---|---|---|---|---|---|---|
| brain | 9.8e−03 | 7.0e−05 | 1.7e−05 | 18.5 | 1.2e−07 | 18.9 |
| ovary | 6.2e−01 | 4.2e−01 | 6.8e−01 | 1.5 | 4.5e−01 | 1.9 |
| bladder | 1.2e−01 | 1.8e−01 | 1.0e−01 | 4.1 | 2.1e−01 | 2.9 |
| lung | 4.6e−01 | 4.9e−01 | 4.1e−01 | 2.4 | 2.3e−01 | 2.7 |
| pancreas | 3.1e−01 | 4.2e−01 | 4.2e−01 | 2.4 | 5.3e−01 | 1.9 |
| liver | N/A | 6.9e−01 | N/A | N/A | 7.0e−01 | 1.4 |
| prostate | 5.0e−01 | 3.1e−01 | 4.5e−01 | 2.0 | 1.8e−01 | 2.7 |
| adrenal | 3.8e−01 | 4.3e−01 | 2.1e−01 | 3.4 | 2.8e−01 | 2.8 |
| general | 6.7e−08 | 5.2e−13 | 1.0e−11 | 8.1 | 8.8e−23 | 10.5 |
| bone marrow | 5.6e−01 | 3.3e−01 | N/A | N/A | 4.4e−02 | 4.6 |
| skin | 3.3e−01 | 4.3e−02 | N/A | N/A | 5.5e−04 | 4.9 |
| muscle | 9.2e−01 | 4.8e−01 | N/A | N/A | 4.0e−01 | 2.3 |
| uterus | 4.0e−01 | 2.2e−01 | 1.3e−01 | 2.5 | 8.5e−02 | 3.0 |
| colon | 2.2e−01 | 1.4e−01 | 4.8e−01 | 1.9 | 3.5e−01 | 2.1 |
| kidney | 3.3e−01 | 2.7e−01 | 5.8e−01 | 1.7 | 4.8e−01 | 1.9 |
| lymph nodes | 6.9e−01 | 5.0e−01 | 1.0e+00 | 0.8 | 1.3e−01 | 1.8 |
| breast | 9.5e−01 | 6.2e−01 | N/A | N/A | 5.6e−01 | 1.2 |
| stomach | 4.6e−01 | 5.5e−01 | 7.5e−01 | 1.0 | 8.1e−01 | 0.9 |
| epithelial | 5.8e−04 | 1.3e−05 | 3.0e−05 | 6.6 | 2.3e−09 | 8.7 |
| bone | N/A | 4.3e−01 | N/A | N/A | 4.9e−01 | 1.9 |

As noted above, cluster T86235 features 10 transcript(s), which were listed in Table 150 above. These transcript(s) encode for protein(s) which are variant(s) of protein Trophinin-associated protein (SEQ ID NO:462). A description of each variant protein according to the present invention is now provided.

Variant protein T86235_P4 (SEQ ID NO:469) according to the present invention has an amino acid sequence encoded by transcript(s) T86235_T13 (SEQ ID NO:444) and T86235_T15 (SEQ ID NO:445). One or more alignments to one or more previously published protein sequences are given in the alignment table in the attached CDROM. A brief description of the relationship of the variant protein according to the present invention to each such aligned protein is as follows:

1. Comparison Report Between T86235_P4 (SEQ ID NO:469) and known protein(s) TAST_HUMAN (SEQ ID NO: 463):

A. An isolated chimeric polypeptide encoding for T86235_P4 (SEQ ID NO:469), comprising a first amino acid sequence being at least about 90% homologous to MTTRQATKDPLLRGVSPTPSKIPVRSQKRTPFPTVTSCAVDQENQDPRRWVQKPPLNIQRPLVDSAGPRPK ARHQAETSQRLVGISQPRNPLEELRPSPRGQNVGPGPPAQTEAPGTIEFVADPAALATILSGEGVKSCHLGR QPSLAKRVLVRGSQGGTTQRVQGVRASAYLAPRTPTHRLDPARASCFSRLEGPGPRGRTLCPQRLQALISP SGPSFHPST corresponding to amino acids 1-223 of known protein(s) TAST_HUMAN (SEQ ID NO: 463), which also corresponds to amino acids 1-223 of T86235_P4 (SEQ ID NO:469), a bridging amino acid R corresponding to amino acid 224 of T86235_P4 (SEQ ID NO:469), a second amino acid sequence being at least about 90% homologous to PSFQELRRETAGSSRTSVSQASGLLLETPVQPAFSLPKGEREVVTHSDEGGVASLGLAQRVPLRENREMSH TRDSHDSHLMPSPAPVAQPLPGHVVPCPSPFGRAQRVPSPGPPTLTSYSVLRRLTVQPKTRFTPMPSTPRVQ QAQWLRGVSPQSCSEDPALPW corresponding to amino acids 225-388 of known protein(s) TAST_HUMAN (SEQ ID NO: 463), which also corresponds to amino acids 225-388 of T86235_P4 (SEQ ID NO:469), and a third amino acid sequence being at least about 70%, optionally at least 80%, preferably at least 85%, more preferably at least 90% and most preferably at least 95% homologous to a polypeptide having the sequence VSIRSFPLLRSLALCCQPGGPGV (SEQ ID NO: 510) corresponding to amino acids 389-411 of T86235_P4 (SEQ ID NO:469), wherein said first amino acid sequence, bridging amino acid, second amino acid sequence and third amino acid sequence are contiguous and in a sequential order.

B. An isolated polypeptide encoding for an edge portion of T86235_P4 (SEQ ID NO:469), comprising an amino acid sequence being at least about 70%, optionally at least about 80%, preferably at least about 85%, more preferably at least about 90% and most preferably at least about 95% homologous to the sequence VSIRSFPLLRSLALCCQPGGPGV (SEQ ID NO: 510) of T86235_P4 (SEQ ID NO:469).

2. Comparison Report Between T86235_P4 (SEQ ID NO:469) and known protein(s) Q8N5B2_HUMAN (SEQ ID NO: 467):

A. An isolated chimeric polypeptide encoding for T86235_P4 (SEQ ID NO:469), comprising a first amino acid sequence being at least about 90% homologous to MTTRQATKDPLLRGVSPTPSKIPVRSQKRTPFPTVTSCAVDQENQDPRRWVQKPPLNIQRPLVDSAGPRPK ARHQAETSQRLVGISQPRNPLEELRPSPRGQNVGPGPPAQTEAPGTIEFVADPAALATILSGEGVKSCHLGR QPSLAKRVLVRGSQGGTTQRVQGVRASAYLAPRTPTHRLDPARASCFSRLEGPGPRGRTLCPQRLQALISP SGPSFHPSTRPSFQELRRETAGSSRTSVSQASGLLLETPVQPAFSLPKGEREVVTHSDEGGVASLGLAQRVP LRENREMSHTRDSHDSHLMPSPAPVAQPLPGHVVPCPSPFGRAQRVPSPGPPTLTSYSVLRRLTVQPKTRFT PMPSTPRVQQAQWLRGVSPQSCSEDPALPW corresponding to amino acids 1-388 of known protein(s) Q8N5B2_HUMAN (SEQ ID NO: 467), which also corresponds to amino acids 1-388 of T86235_P4 (SEQ ID NO:469), and a second amino acid sequence being at least about 70%, optionally at least 80%, preferably at least 85%, more preferably at least 90% and most preferably at least 95% homologous to a polypeptide having the sequence VSIRSFPLLRSLALCCQPGGPGV (SEQ ID NO: 510) corresponding to amino acids 389-411 of T86235_P4 (SEQ ID NO:469), wherein said first amino acid sequence and second amino acid sequence are contiguous and in a sequential order.

3. Comparison Report Between T86235_P4 (SEQ ID NO:469) and known protein(s) Q6PJU7_HUMAN (SEQ ID NO: 439):

A. An isolated chimeric polypeptide encoding for T86235_P4 (SEQ ID NO:469), comprising a first amino acid sequence being at least about 90% homologous to MTTRQATKDPLLRGVSPTPSKIPVRSQKRTPFPTVTSCAVDQENQDPRRWVQKPPLNIQRPLVDSAGPRPK ARHQAETSQRLVGISQPRNPLEELRPSPRGQNVGPGPPAQT corresponding to amino acids 1-112 of known protein(s) Q6PJU7_HUMAN (SEQ ID NO: 439), which also corresponds to amino acids 1-112 of T86235_P4 (SEQ ID NO:469), and a second amino acid sequence being at least about 70%, optionally at least 80%, preferably at least 85%, more preferably at least 90% and most preferably at least 95% homologous to a polypeptide having the sequence EAPGTIEFVADPAALATILSGEGVKSCHLGRQPSLAKRVLVRGSQGGTTQRVQGVRASAYLAPRTP THRLDPARASCFSRLEGPGPRGRTLCPQRLQALISPSGPSFHPSTRPSFQELRRETAGSSRTSVSQASGLLLET PVQPAFSLPKGEREVVTHSDEGGVASLGLAQRVPLRENREMSHTRDSHDSHLMPSPAPVAQPLPGHVVPC PSPFGRAQRVPSPGPPTLTSYSVLRRLTVQPKTRFTPMPSTPRVQQAQWLRGVSPQSCSEDPALPWVSIRSF PLLRSLALCCQPGGPGV (SEQ ID NO: 511) corresponding to amino acids 113-411 of T86235_P4 (SEQ ID NO:469), wherein said first amino acid sequence and second amino acid sequence are contiguous and in a sequential order.

B. An isolated polypeptide encoding for an edge portion of T86235_P4 (SEQ ID NO:469), comprising an amino acid sequence being at least about 70%, optionally at least about 80%, preferably at least about 85%, more preferably at least about 90% and most preferably at least about 95% homologous to the sequence EAPGTIEFVADPAALATILSGEGVKSCHLGRQPSLAKRVLVRGSQGGTTQRVQGVRASAYLAPRTPTHRLD PARASCFSRLEGPGPRGRTLCPQRLQALISPSGPSFHPSTRPSFQELRRETAGSSRTSVSQASGLLLETPVQPA FSLPKGEREVVTHSDEGGVASLGLAQRVPLRENREMSHTRDSHDSHLMPSPAPVAQPLPGHVVPCPSPFGR AQRVPSPGPPTLTSYSVLRRLTVQPKTRFTPMPSTPRVQQAQWLRGVSPQSCSEDPALPWVSIRSFPLLRSL ALCCQPGGPGV (SEQ ID NO: 511) of T86235_P4 (SEQ ID NO:469).

The localization of the variant protein was determined according to results from a number of different software programs and analyses, including analyses from SignalP and other specialized programs. The variant protein is believed to be located intracellularly with regard to the cell.

Variant protein T86235_P4 (SEQ ID NO:469) also has the following non-silent SNPs (Single Nucleotide Polymorphisms) as listed in Table 155, (given according to their position(s) on the amino acid sequence, with the alternative amino acid(s) listed).

TABLE 155
Amino acid mutations

| SNP position(s) on amino acid | |
|---|---|
| sequence | Alternative amino acid(s) |
| 82 | L -> |
| 116 | G -> |
| 120 | F -> |
| 165 | Q -> |
| 242 | V -> A |
| 293 | M -> V |
| 333 | P -> L |

Variant protein T86235_P4 (SEQ ID NO:469) is encoded by the following transcript(s): T86235_T13 (SEQ ID NO:444) and T86235—T15 (SEQ ID NO:445), for which the sequences are given.

The coding portion of transcript T86235_T13 (SEQ ID NO:444) starts at position 298 and ends at position 1530. The transcript also has the following SNPs as listed in Table 156 (given according to their position on the nucleotide sequence, with the alternative nucleic acid listed).

TABLE 156
Nucleic acid SNPs

| Polymorphism | SNP position(s) on nucleotide sequence |
|---|---|
| G -> | 240, 643, 792, 2864 |
| G -> A | 360, 1805, 2259, 3127 |
| A -> C | 439 |
| T -> | 541, 655, 2527 |
| C -> T | 909, 1272, 1295, 1374, 1874 |
| T -> C | 1022, 2445 |
| A -> G | 1174, 1911, 2187, 2536 |
| C -> G | 1660, 2126 |
| C -> | 2710 |

The coding portion of transcript T86235_T15 (SEQ ID NO:445) starts at position 298 and ends at position 1530. The transcript also has the following SNPs as listed in Table 157 (given according to their position on the nucleotide sequence, with the alternative nucleic acid listed).

TABLE 157
Nucleic acid SNPs

| Polymorphism | SNP position(s) on nucleotide sequence |
|---|---|
| G -> | 240, 643, 792, 3068 |
| G -> A | 360, 1805, 2106, 3331 |
| A -> C | 439, 3026 |
| T -> | 541, 655, 2374 |
| C -> T | 909, 1272, 1295, 1374, 1874 |
| T -> C | 1022, 2292 |
| A -> G | 1174, 1911, 2034, 2383 |
| C -> G | 1660 |
| C -> | 2644 |

Variant protein T86235_P5 (SEQ ID NO:470) according to the present invention has an amino acid sequence encoded by transcript T86235_T5 (SEQ ID NO:440). An alignment is given to the known protein (Trophinin-associated protein, SEQ ID NO:462-468) at the end of the application. One or more alignments to one or more previously published protein sequences are given in the alignment table on the attached CDROM. A brief description of the relationship of the variant protein according to the present invention to each such aligned protein is as follows:

1. Comparison Report Between T86235_P5 (SEQ ID NO:470) and known protein(s) TAST_HUMAN (SEQ ID NO: 463):

A. An isolated chimeric polypeptide encoding for T86235_P5 (SEQ ID NO:470), comprising a first amino acid sequence being at least about 90% homologous to MTTRQATKDPLLRGVSPTPSKIPVRSQKRTPFPTVTSCAVDQENQDPRRWVQKPPLNIQRPLVDSAGPRPK ARHQAETSQRLVGISQPRNPLEELRPSPRGQNVGPGPPAQTEAPGTIEFVADPAALATILSGEGVKSCHLGR QPSLAKRVLVRGSQGGTTQRVQGVRASAYLAPRTPTBRLDPARASCFSRLEGPGPRGRTLCPQRLQALISP SGPSFHPST corresponding to amino acids 1-223 of known protein(s) TAST_HUMAN (SEQ ID NO: 463), which also corresponds to amino acids 1-223 of T86235_P5 (SEQ ID NO:470), a bridging amino acid R corresponding to amino acid 224 of T86235_P5 (SEQ ID NO:470), a second amino acid sequence being at least about 90% homologous to

PSFQELRRETAGSSRTSVSQASGLLLETPVQPAFSLPKGEREVVTHSDEG
GVASLGLAQRVPLRENREMSHTRDSHDSHLMPSPAPVAQPLPGHVVPCPS
PFGRAQRVPSPGPPTLTSYSVLRRLTVQPKTRFTPMPSTPRVQQAQWLRG
VSPQSCSEDPALPWEQVAVRLFDQESCIRSLEGSGKPPVATPSGPHSNRT
PSLQEVKIQ

corresponding to amino acids 225-433 of known protein(s) TAST_HUMAN (SEQ ID NO: 463), which also corresponds to amino acids 225-433 of T86235_P5 (SEQ ID NO:470), and a third amino acid sequence being at least about 70%, optionally at least 80%, preferably at least 85%, more preferably at least 90% and most preferably at least 95% homologous to a polypeptide having the sequence VSLCGQQL (SEQ ID NO: 512) corresponding to amino acids 434-441 of T86235_P5 (SEQ ID NO:470), wherein said first amino acid sequence, bridging amino acid, second amino acid sequence and third amino acid sequence are contiguous and in a sequential order.

B. An isolated polypeptide encoding for an edge portion of T86235_P5 (SEQ ID NO:470), comprising an amino acid sequence being at least about 70%, optionally at least about 80%, preferably at least about 85%, more preferably at least about 90% and most preferably at least about 95% homologous to the sequence VSLCGQQL (SEQ ID NO: 512) of T86235_P5 (SEQ ID NO:470).

2. Comparison Report Between T86235_P5 (SEQ ID NO:470) and known protein(s) Q8N5B2_HUMAN (SEQ ID NO: 467):

A. An isolated chimeric polypeptide encoding for T86235_P5 (SEQ ID NO:470), comprising a first amino acid sequence being at least about 90% homologous to MTTRQATKDPLLRGVSPTPSKIPVRSQKRTPFPTVTSCAVDQENQDPRRWVQKPPLNIQRPLVDSAGPRPK ARHQAETSQRLVGISQPRNPLEELRPSPRGQNVGPGPPAQTEAPGTIEFVADPAALATILSGEGVKSCHLGR QPSLAKRVLVRGSQGGTTQRVQGVRASAYLAPRTPTHRLDPARASCFSRLEGPGPRGRTLCPQRLQALISP SGPSFHPSTRPSFQELRRETAGSSRTSVSQASGLLLETPVQPAFSLPKGEREVVTHSDEGGVASLGLAQRVP LRENREMSHTRDSHDSHLMPSPAPVAQPLPGHVVPCPSPFGRAQRVPSPGPPTLTSYSVLRRLTVQPKTRFT PMPSTPRVQQAQWLRGVSPQSCSEDPALPWEQVAVRLFDQESCIRSLEGSGKPPVATPSGPHSNRTPSLQE VKIQ corresponding to amino acids 1-433 of known protein(s) Q8N5B2_HUMAN (SEQ ID NO: 467), which also corresponds to amino acids 1-433 of T86235_P5 (SEQ ID NO:470), and a second amino acid sequence being at least about 70%, optionally at least 80%, preferably at least 85%, more preferably at least 90% and most preferably at least 95% homologous to a polypeptide having the sequence VSLCGQQL (SEQ ID NO: 512) corresponding to amino acids 434-441 of T86235_P5 (SEQ ID NO:470), wherein said first amino acid sequence and second amino acid sequence are contiguous and in a sequential order.

3. Comparison Report Between T86235_P5 (SEQ ID NO:470) and known protein(s) Q6PJU7_HUMAN (SEQ ID NO: 439):

A. An isolated chimeric polypeptide encoding for T86235_P5 (SEQ ID NO:470), comprising a first amino acid sequence being at least about 90% homologous to MTTRQATKDPLLRGVSPTPSKIPVRSQKRTPFPTVTSCAVDQENQDPRRWVQKPPLNIQRPLVDSAGPRPK ARHQAETSQRLVGISQPRNPLEELRPSPRGQNVGPGPPAQT corresponding to amino acids 1-112 of known protein(s) Q6PJU7_HUMAN (SEQ ID NO: 439), which also corresponds to amino acids 1-112 of T86235_P5 (SEQ ID NO:470), and a second amino acid sequence being at least about 70%, optionally at least 80%, preferably at least 85%, more preferably at least 90% and most preferably at least 95% homologous to a polypeptide having the sequence EAPGTIEFVADPAALATILSGEGVKSCJHLGRQPSLAKRVLVRGSQGGTTQRVQGVRASAYLAPRTPTHRLD PARASCFSRLEGPGPRGRTLCPQRLQALISPSGPSFHPSTRPSFQELRRETAGSSRTSVSQASGLLLETPVQPA FSLPKGEREVVTHSDEGGVASLGLAQRVPLRENREMSHTRDSHDSHLMPSPAPVAQPLPGHVVPCPSPFGR AQRVPSPGPPTLTSYSVLRRLTVQPKTRFTPMPSTPRVQQAQWLRGVSPQSCSEDPALPWEQVAVRLFDQE SCIRSLEGSGKPPVATPSGPHSNRTPSLQEVKIQVSLCGQQL (SEQ ID NO: 513) corresponding to amino acids 113-441 of T86235_P5 (SEQ ID NO:470), wherein said first amino acid sequence and second amino acid sequence are contiguous and in a sequential order.

B. An isolated polypeptide encoding for an edge portion of T86235_P5 (SEQ ID NO:470), comprising an amino acid sequence being at least about 70%, optionally at least about 80%, preferably at least about 85%, more preferably at least about 90% and most preferably at least about 95% homologous to the sequence

(SEQ ID NO: 513)

EAPGTIEFVADPAALATILSGEGVKSCHLGRQPSLAKRVLVRGSQGGTTQ
RVQGVRASAYLAPRTPTHRLDPARASCFSRLEGPGPRGRTLCPQRLQALI
SPSGPSFHPSTRPSFQELRRETAGSSRTSVSQASGLLLETPVQPAFSLPK
GEREVVTHSDEGGVASLGLAQRVPLRENREMSHTRDSHDSHLMPSPAPVA
QPLPGHVVPCPSPFGRAQRVPSPGPPTLTSYSVLRRLTVQPKTRFTPMPS
TPRVQQAQWLRGVSPQSCSEDPALPWEQVAVRLFDQESCIRSLEGSGKPP
VATPSGPHSNRTPSLQEVKIQVSLCGQQL
of

(SEQ ID NO: 470)

T86235_P5.

The localization of the variant protein was determined according to results from a number of different software programs and analyses, including analyses from SignalP and other specialized programs. The variant protein is believed to be located intracellularly with regard to the cell.

Variant protein T86235_P5 (SEQ ID NO:470) also has the following non-silent SNPs (Single Nucleotide Polymorphisms) as listed in Table 158, (given according to their position(s) on the amino acid sequence, with the alternative amino acid(s) listed).

TABLE 158
Amino acid mutations

| SNP position(s) on amino acid | |
|---|---|
| sequence | Alternative amino acid(s) |
| 82 | L -> |
| 116 | G -> |
| 120 | F -> |
| 165 | Q -> |
| 242 | V -> A |
| 293 | M -> V |
| 333 | P -> L |
| 392 | A -> V |

Variant protein T86235_P5 (SEQ ID NO:470) is encoded by the following transcript(s): T86235_T5 (SEQ ID NO:440), for which the coding portion of transcript T86235_T5 (SEQ ID NO:440) starts at position 298 and ends at position 1620. The transcript also has the following SNPs as listed in Table 159 (given according to their position on the nucleotide sequence, with the alternative nucleic acid listed).

TABLE 159
Nucleic acid SNPs

| Polymorphism | SNP position(s) on nucleotide sequence |
|---|---|
| G -> | 240, 643, 792, 2462 |
| G -> A | 360, 1857, 2725 |
| A -> C | 439 |
| T -> | 541, 655, 2125 |
| C -> T | 909, 1272, 1295, 1374, 1472 |
| T -> C | 1022, 2043 |
| A -> G | 1174, 1509, 1785, 2134 |
| C -> G | 1724 |
| C -> | 2308 |

Variant protein T86235_P6 (SEQ ID NO:471) according to the present invention has an amino acid sequence encoded by transcript(s) T86235_T6 (SEQ ID NO:441). One or more alignments to one or more previously published protein sequences are given in the alignment table on the attached CDROM. A brief description of the relationship of the variant protein according to the present invention to each such aligned protein is as follows:

1. Comparison Report Between T86235_P6 (SEQ ID NO:471) and known protein(s) TAST_HUMAN (SEQ ID NO: 463):

A. An isolated chimeric polypeptide encoding for T86235_P6 (SEQ ID NO:471), comprising a first amino acid sequence being at least about 90% homologous to MTTRQATKDPLLRGVSPTPSKIPVRSQKRTPFPTVTSCAVDQENQDPRRWVQKPPLNIQRPLVDSAGPRPK ARHQAETSQRLVGISQPRNPLEELRPSPRGQNVGPGPPAQTEAPGTIEFVADPAALATILSGEGVKSCHLGR QPSLAKRVLVRGSQGGTTQRVQGVRASAYLAPRTPTBRLDPARASCFSRLEGPGPRGRTLCPQRLQALISP SGPSFHPST corresponding to amino acids 1-223 of known protein(s) TAST_HUMAN (SEQ ID NO: 463), which also corresponds to amino acids 1-223 of T86235_P6 (SEQ ID NO:471), a bridging amino acid R corresponding to amino acid 224 of T86235_P6 (SEQ ID NO:471), a second amino acid sequence being at least about 90% homologous to PSFQELRRETAGSSRTSVSQASGLLLETPVQPAFSLPKGEREVVTHSDEGGVASLGLAQRVPLRENREMSH TRDSHDSHLMPSPAPVAQPLPGHVVPCPSPFGRAQRVPSPGPPTLTSYSVLRRLTVQPKTRFTPMPSTPRVQ QAQWLRGVSPQSCSEDPALPWEQVAVRLFDQESCIRSLEGSGKPPVATPSGPHSNRTPSLQEVKIQRIGILQ QLLRQEVEGLVGGQCVPLNGGSSLDMVELQPLLTEISRTLNATEHNSGTSHLPGLLKHSGLPKPCLPEECG EPQPCPPAEPGPPEAFCRSEPEIPEPSLQEQLEVPEPYPPAEPRPLESCCRSEPEIPESSRQEQLE corresponding to amino acids 225-576 of known protein(s) TAST_HUMAN (SEQ ID NO: 463), which also corresponds to amino acids 225-576 of T86235_P6 (SEQ ID NO:471), a third amino acid sequence being at least about 90% homologous to EQLEVPEPCPPAEPGPLQPSTQGQSGPPGPCPRVELGASEPCTLEHRSLESSLPPCCSQWAPATTSLIFSSQHP LCASPPICSLQSLRPPAGQA corresponding to amino acids 606-699 of known protein(s) TAST_HUMAN (SEQ ID NO: 463) which also corresponds to amino acids 577-670 of T86235_P6 (SEQ ID NO:471), a fourth amino acid sequence being at least about 70%, optionally at least 80%, preferably at least 85%, more preferably at least 90% and most preferably at least 95% homologous to a polypeptide having the sequence GKELAGKECEHKRSSPHCELHTCPAPTPGNLMLPHLPPWPSLALPQEEGRGCTSSPVLLIGLAVGGGGGED STWWKYRTPDLPLNFPCPS (SEQ ID NO: 514) corresponding to amino acids 671-760 of T86235_P6 (SEQ ID NO:471), and a fifth amino acid sequence being at least about 90% homologous to GLSNLAPRTLALRERLKSCLTAIHCFHEARLDDECAFYTSRAPPSGP_RVCTNPVATLLEWQDALCFIPVGS AAPQGSP corresponding to amino acids 700-778 of known protein(s) TAST_HUMAN (SEQ ID NO: 463), which also corresponds to amino acids 761-839 of T86235_P6 (SEQ ID NO:471), wherein said first amino acid sequence, bridging amino acid, second amino acid sequence, third amino acid sequence, fourth amino acid sequence and fifth amino acid sequence are contiguous and in a sequential order.

B. An isolated chimeric polypeptide encoding for an edge portion of T86235_P6 (SEQ ID NO:471), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EE, having a structure as follows: a sequence starting from any of amino acid numbers 576−x to 576; and ending at any of amino acid numbers 577+((n−2)−x), in which x varies from 0 to n−2.

2. Comparison Report Between T86235_P6 (SEQ ID NO:471) and known protein(s) Q8N5B2_HUMAN (SEQ ID NO: 467):

A. An isolated chimeric polypeptide encoding for T86235_P6 (SEQ ID NO:471), comprising a first amino acid sequence being at least about 90% homologous to

MTTRQATKDPLLRGVSPTPSKIPVRSQKRTPFPTVTSCAVDQENQDPRRW
VQKPPLNIQRPLVDSAGPRPKAHQAETSQRLVGISQPRNPLEELRPSPRG
QNVGPGPPAQTEAPGTIEFVADPAALATILSGEGVKSCHLGRQPSLAKRV
LVRGSQGGTTQRVQGVRASAYLAPRTPTHRLDPARASCFSRLEGPGPRGR
TLCPQRLQALISPSGPSFHPSTRPSFQELRRETAGSSRTSVSQASGLLLE
TPVQPAFSLPKGEREVVTHSDEGGVASLGLAQRVPLRENREMSHTRDSHD
SHLMSPAPVAQPLPGHVVPCPSPFGRAQRVPSPGPPTLTSYSVLRRLTVQ
PKTRFTPMPSTPRVQQAQWLRGVSPQSCSEDPALPWEQVAVRLFDQESCI
RSLEGSGKPPVATPSGPHSNRTPSLQEVKIQRIGILQQLLRQEVEGLVGG
QCVPLNGGSSLDMVELQPLLTEISRTLNATEHNSGTSHLPGLLKHSGLPK
PCLPEECGEPQPCPPAEPGPPEAFCRSEPEIPEPSLQEQLEVPEPYPPAE
PRPLESCCRSEPEIPESSRQEQLE

corresponding to amino acids 1-576 of known protein(s) Q8N5B2_HUMAN (SEQ ID NO: 467), which also corresponds to amino acids 1-576 of T86235_P6 (SEQ ID NO:471), a second amino acid sequence being at least about 90% homologous to EQLEVPEPCPPAEPGPLQPSTQGQSGPPGPCPRVELGASEPCTLEHRSLESSLPPCCSQWAPATTSLIFSSQHP LCASPPICSLQSLRPPAGQA corresponding to amino acids 606-699 of known protein(s) Q8N5B2_HUMAN (SEQ ID NO: 467), which also corresponds to amino acids 577-670 of T86235_P6 (SEQ ID NO:471), a third amino acid sequence being at least about 70%, optionally at least 80%, preferably at least 85%, more preferably at least 90% and most preferably at least 95% homologous to a polypeptide having the sequence GKELAGKECEHKRSSPHCELHTCPAPTPGNLMLPHLPPWPSLALPQEEGRGCTSSPVLLIGLAVGGGGGED STWWKYRTPDLPLNFPCPS (SEQ ID NO: 514) corresponding to amino acids 671-760 of T86235_P6 (SEQ ID NO:471), and a fourth amino acid sequence being at least about 90% homologous to GLSNLAPRTLALRERLKSCLTAIHCFHEARLDDECAFYTSRAPPSGPTRVCTNPVATLLEWQDALCFIPVGS APQGSP corresponding to amino acids 700-778 of known protein(s) Q8N5B2_HUMAN (SEQ ID NO: 467), which also corresponds to amino acids 761-839 of T86235_P6 (SEQ ID NO:471), wherein said first amino acid sequence, second amino acid sequence, third amino acid sequence and fourth amino acid sequence are contiguous and in a sequential order.

B. An isolated chimeric polypeptide encoding for an edge portion of T86235_P6 (SEQ ID NO:471), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EE, having a structure as follows: a sequence starting from any of amino acid numbers 576−x to 576; and ending at any of amino acid numbers 577+((n−2)−x), in which x varies from 0 to n−2.

C. An isolated polypeptide encoding for an edge portion of T86235_P6 (SEQ ID NO:471), comprising an amino acid sequence being at least about 70%, optionally at least about 80%, preferably at least about 85%, more preferably at least about 90% and most preferably at least about 95% homologous to the sequence

(SEQ ID NO: 514)

GKELAGKECEHKRSSPHCELHTCPAPTPGNLMLPHLPPWPSLALPQEEGR
GCTSSPVLLIGLAVGGGGGEDSTWWKYRTPDLPLNFPCPS
of

(SEQ ID NO: 471)

T86235_P6.

3. Comparison Report Between T86235_P6 (SEQ ID NO:471) and known protein(s) Q6PJU7_HUMAN (SEQ ID NO: 439):

A. An isolated chimeric polypeptide encoding for T86235_P6 (SEQ ID NO:471), comprising a first amino acid sequence being at least about 90% homologous to MTTRQATKDPLLRGVSPTPSKIPVRSQKRTPFPTVTSCAVDQENQDPRRWVQKPPLNIQRPLVDSAGPRPK ARHQAETSQRLVGISQPRNPLEELRPSPRGQNVGPGPPAQT corresponding to amino acids 1-112 of known protein(s) Q6PJU7_HUMAN (SEQ ID NO: 439), which also corresponds to amino acids 1-112 of T86235_P6 (SEQ ID NO:471), and a second amino acid sequence being at least about 70%, optionally at least 80%, preferably at least 85%, more preferably at least 90% and most preferably at least 95% homologous to a polypeptide having the sequence EAPGTIEFVADPAALATILSGEGVKSCHLGRQPSLAKRVLVRGSQGGTTQRVQGVRASAYLAPRTPTBRLD PARASCFSRLEGPGPRGRTLCPQRLQALISPSGPSFHPSTRPSFQELRRETAGSSRTSVSQASGLLLETPVQPA FSLPKGEREVVTHSDEGGVASLGLAQRVPLRENREMSHTRDSHDSHLMPSPAPVAQPLPGIIVVPCPSPFGR AQRVPSPGPPTLTSYSVLRRLTVQPKTRFTPMPSTPRVQQAQWLRGVSPQSCSEDPALPWEQVAVRLFDQE SCIRSLEGSGKPPVATPSGPHSNRTPSLQEVKIQRIGILQQLLRQEVEGLVGGQCVPLNGGSSLDMVELQPL LTEISRTLNATEHNSGTSHLPGLLKHSGLPKPCLPEECGEPQPCPPAEPGPPEAFCRSEPEIPEPSLQEQLEVP EPYPPAEPRPLESCCRSEPEIPESSRQEQLEEQLEVPEPCPPAEPGPLQPSTQGQSGPPGPCPRVELGASEPCT LEHRSLESSLPPCCSQWAPATTSLIFSSQHPLCASPPICSLQSLRPPAGQAGKELAGKECEHKRSSPHCELHT CPAPTPGNLMLPHLPPWPSLALPQEEGRGCTSSPVLLIGLAVGGGGGEDSTWWKYRTPDLPLNFPCPSGLS NLAPRTLALRERLKSCLTAIHCFHEARLDDECAFYTSRAPPSGPTRVCTNPVATLLEWQDALCFIPVGSAAP QGSP (SEQ ID NO: 515) corresponding to amino acids 113-839 of T86235_P6 (SEQ ID NO:471), wherein said first amino acid sequence and second amino acid sequence are contiguous and in a sequential order.

B. An isolated polypeptide encoding for an edge portion of T86235_P6 (SEQ ID NO:471), comprising an amino acid sequence being at least about 70%, optionally at least about 80%, preferably at least about 85%, more preferably at least about 90% and most preferably at least about 95% homologous to the sequence

(SEQ ID NO: 515)

EAPGTIEFVADPAALATILSGEGVKSCHLGRQPSLAKRVLVRGSQGGTTQ
RVQGVRASAYLAPRTPTHRLDPARASCFSRLEGPGPRGRTLCPQRLQALI
SPSGPSFHPSTRPSFQELRRETAGSSRTSVSQASGLLLETPVQPAFSLPK
GEREVVTHSDEGGVASLGLAQRVPLRENREMSHTRDSHDSHLMPSPAPVA
QPLPGHVVPCPSPFGRAQRVPSPGPPTLTSYSVLRRLTVQPKTRFTPMPS
TPRVQQAQWLRGVSPQSCSEDPALPWEQVAVRLFDQESCIRSLEGSGKPP
VATPSGPHSNRTPSLQEVKIQRIGILQQLLRQEVEGLVGGQCVPLNGGSS
LDMVELQPLLTEISRTLNATEHNSGTSHLPGLLKHSGLPKPCLPEECGEP
QPCPPAEPGPPEAFCRSEPELPEPSLQEQLEVPEPYPPAEPRPLESGCRS
EPEIPESSRQEQLEEQLEVPEPCPPAEPGPLQPSTQGQSGPPGPCPRVEL
GASEPCTLEHRSLESSLPPCCSQWAPATTSLLFSSQHPLCASPPICSLQS
LRPPAGQAGKELAGKEGEHKRSSPHCELHTCPAPTPGNLMLPHLPPWPSL
ALPQEEGRGCTSSPVLLIGLAVGGGGGEDSTWWKYRTPDLPLNFPCPSGL
SNLAPRTLALRERLKSCLTAIHCFHEARILDDECAFYTSRAPPSGPTRVC
TNPVATLLEWQDALCFIPVGSAAPQGSP
of

(SEQ ID NO: 471)

T86235_P6.

The localization of the variant protein was determined according to results from a number of different software programs and analyses, including analyses from SignalP and other specialized programs. The variant protein is believed to be located intracellularly with regard to the cell.

Variant protein T86235_P6 (SEQ ID NO:471) also has the following non-silent SNPs (Single Nucleotide Polymorphisms) as listed in Table 160, (given according to their position(s) on the amino acid sequence, with the alternative amino acid(s) listed).

TABLE 160
Amino acid mutations

| SNP position(s) on amino acid | |
|---|---|
| sequence | Alternative amino acid(s) |
| 82 | L -> |
| 116 | G -> |
| 120 | F -> |
| 165 | Q -> |
| 242 | V -> A |
| 293 | M -> V |
| 333 | P -> L |
| 392 | A -> V |
| 559 | C -> |
| 562 | S -> G |
| 620 | L -> |
| 747 | Y -> S |
| 761 | G -> |

Variant protein T86235_P6 (SEQ ID NO:471) is encoded by the following transcript(s): T86235_T6 (SEQ ID NO:441), for which the coding portion starts at position 298 and ends at position 2814. The transcript also has the following SNPs as listed in Table 161 (given according to their position on the nucleotide sequence, with the alternative nucleic acid listed).

TABLE 161
Nucleic acid SNPs

| Polymorphism | SNP position(s) on nucleotide sequence |
|---|---|
| G -> | 240, 643, 792, 2579 |
| G -> A | 360, 1704, 2842 |
| A -> C | 439, 2537 |
| T -> | 541, 655, 1972 |
| C -> T | 909, 1272, 1295, 1374, 1472 |
| T -> C | 1022, 1890 |
| A -> G | 1174, 1509, 1632, 1981 |
| C -> | 2155 |

Variant protein T86235_P7 (SEQ ID NO:472) according to the present invention has an amino acid sequence encoded by transcript(s) T86235_T7 (SEQ ID NO:442). One or more alignments to one or more previously published protein sequences are given in the alignment table on the attached CDROM. A brief description of the relationship of the variant protein according to the present invention to each such aligned protein is as follows:

1. Comparison Report Between T86235_P7 (SEQ ID NO:472) and known protein(s) TAST_HUMAN (SEQ ID NO: 463):

A. An isolated chimeric polypeptide encoding for T86235_P7 (SEQ ID NO:472), comprising a first amino acid sequence being at least about 90% homologous to MTTRQATKDPLLRGVSPTPSKIPVRSQKRTPFPTVTSCAVDQENQDPRRWVQKPPLNIQRPLVDSAGPRPK ARHQAETSQRLVGISQPRNPLEELRPSPRGQNVGPGPPAQTEAPGTIEFVADPAALATILSGEGVKSCHLGR QPSLAKRVLVRGSQGGTTQRVQGVRASAYLAPRTPTHRLDPARASCFSRLEGPGPRGRTLCPQRLQALISP SGPSFHPST corresponding to amino acids 1-223 of known protein(s) TAST_HUMAN (SEQ ID NO: 463), which also corresponds to amino acids 1-223 of T86235_P7 (SEQ ID NO:472), a bridging amino acid R corresponding to amino acid 224 of T86235_P7 (SEQ ID NO:472), a second amino acid sequence being at least about 90% homologous to PSFQELRRETAGSSRTSVSQASGLLLETPVQPAFSLPKGEREVVTHSDEGGVASLGLAQRVPLRENREMSH TRDSHDSHLMPSPAPVAQPLPGHVVPCPSPFGRAQRVPSPGPPTLTSYSVLRRLTVQPKTRFTPMPSTPRVQ QAQWLRGVSPQSCSEDPALPWEQVAVRLFDQESCIRSLEGSGKPPVATPSGPHSNRTPSLQEVKIQRIGILQ QLLRQEVEGLVGGQCVPLNGGSSLDMVELQPLLTEISRTLNATEHNSGTSHLPGLLKHSGLPKPCLPEECG EPQPCPPAEPGPPEAFCRSEPEIPEPSLQEQLEVPEPYPPAEPRPLESCCRSEPEIPESSRQEQLEVPEPCPPAEP RPLESYCRIEPEIPESSRQEQLEVPEPCPPAEPGPLQPSTQGQSGPPGPCPRVELGASEPCTLEHRSLESSLPPC CSQWAPATTSLIFSSQHPLCASPPICSLQSLRPPAGQA corresponding to amino acids 225-699 of known protein(s) TAST_HUMAN (SEQ ID NO: 463), which also corresponds to amino acids 225-699 of T86235_P7 (SEQ ID NO:472), a third amino acid sequence being at least about 70%, optionally at least 80%, preferably at least 85%, more preferably at least 90% and most preferably at least 95% homologous to a polypeptide having the sequence GKELAGKECEHKRSSPHCELHTCPAPTPGNLMLPHLPPWPSLALPQEEGRGCTSSPVLLIGLAVGGGGGED STWWKYRTPDLPLNFPCPS (SEQ ID NO: 514) corresponding to amino acids 700-789 of T86235_P7 (SEQ ID NO:472), and a fourth amino acid sequence being at least about 90% homologous to GLSNLAPRTLALRERLKSCLTAIHCFHEARLDDECAFYTSRAPPSGPTRVCTNPVATLLEWQDALCFIPVGS AAPQGSP corresponding to amino acids 700-778 of known protein(s) TAST_HUMAN (SEQ ID NO: 463), which also corresponds to amino acids 790-868 of T86235_P7 (SEQ ID NO:472), wherein said first amino acid sequence, bridging amino acid, second amino acid sequence, third amino acid sequence and fourth amino acid sequence are contiguous and in a sequential order.

B. An isolated polypeptide encoding for an edge portion of T86235_P7 (SEQ ID NO:472), comprising an amino acid sequence being at least about 70%, optionally at least about 80%, preferably at least about 85%, more preferably at least about 90% and most preferably at least about 95% homologous to the sequence

(SEQ ID NO: 514)

GKELAGKECEHKRSSPHCELHTCPAPTPGNLMLPHLPPWPSLALPQEEGR
GCTSSPVLLIGLAVGGGGGEDSTWWKYRTPDLPLNFPCPS
of

(SEQ ID NO: 472)

T86235_P7.

2. Comparison Report Between T86235_P7 (SEQ ID NO:472) and known protein(s) Q8N5B2_HUMAN (SEQ ID NO: 467):

A. An isolated chimeric polypeptide encoding for T86235_P7 (SEQ ID NO:472), comprising a first amino acid sequence being at least about 90% homologous to MTTRQATKDPLLRGVSPTPSKIPVRSQKRTPFPTVTSCAVDQENQDPRRWVQKPPLNIQRPLVDSAGPRPK ARHQAETSQRLVGISQPRNPLEELRPSPRGQNVGPGPPAQTEAPGTIEFVADPAALATILSGEGVKSCHLGR QPSLAKRVLVRGSQGGTTQRVQGVRASAYLAPRTPTBRLDPARASCFSRLEGPGPRGRTLCPQRLQALISP SGPSFHPSTRPSFQELRRETAGSSRTSVSQASGLLLETPVQPAFSLPKGEREVVTHSDEGGVASLGLAQRVP LRENREMSHTRDSHDSHLMPSPAPVAQPLPGHVVPCPSPFGRAQRVPSPGPPTLTSYSVLRRLTVQPKTRFT PMPSTPRVQQAQWLRGVSPQSCSEDPALPWEQVAVRLFDQESCIRSLEGSGKPPVATPSGPHSNRTPSLQE VKIQRIGILQQLLRQEVEGLVGGQCVPLNGGSSLDMVELQPLLTEISRTLNATEHNSGTSHLPGLLKHSGLP KPCLPEECGEPQPCPPAEPGPPEAFCRSEPEIPEPSLQEQLEVPEPYPPAEPRPLESCCRSEPEIPESSRQEQLE VPEPCPPAEPRPLESYCRIEPEIPESSRQEQLEVPEPCPPAEPGPLQPSTQGQSGPPGPCPRVELGASEPCTLEH RSLESSLPPCCSQWAPATTSLIFSSQHPLCASPPICSLQSLRPPAGQA corresponding to amino acids 1-699 of known protein(s) Q8N5B2_HUMAN (SEQ ID NO: 467), which also corresponds to amino acids 1-699 of T86235_P7 (SEQ ID NO:472), a second amino acid sequence being at least about 70%, optionally at least 80%, preferably at least 85%, more preferably at least 90% and most preferably at least 95% homologous to a polypeptide having the sequence GKELAGKECEHKRSSPHCELHTCPAPTPGNLMLPHLPPWPSLALPQEEGRGCTSSPVLLIGLAVGGGGGED STWWKYRTPDLPLNFPCPS (SEQ ID NO: 514) corresponding to amino acids 700-789 of T86235_P7 (SEQ ID NO:472), and a third amino acid sequence being at least about 90% homologous to GLSNLAPRTLALRERLKSCLTAIHCFHEARLDDECAFYTSRAPPSGPTRVCTNPVATLLEWQDALCFIPVGS AAPQGSP corresponding to amino acids 700-778 of known protein(s) Q8N5B2_HUMAN (SEQ ID NO: 467), which also corresponds to amino acids 790-868 of T86235_P7 (SEQ ID NO:472), wherein said first amino acid sequence, second amino acid sequence and third amino acid sequence are contiguous and in a sequential order.

B. An isolated polypeptide encoding for an edge portion of T86235_P7 (SEQ ID NO:472), comprising an amino acid sequence being at least about 70%, optionally at least about 80%, preferably at least about 85%, more preferably at least about 90% and most preferably at least about 95% homologous to the sequence

(SEQ ID NO: 514)

GKELAGKECEHKRSSPHCELHTCPAPTPGNLMLPHLPPWPSLALPQEEGR
GCTSSPVLLIGLAVGGGGGEDSTWWKYRTPDLPLNFPCPS
of

(SEQ ID NO: 472)

T86235_P7.

### 3. Comparison Report Between T86235_P7 (SEQ ID NO:472) and Known Protein(s) Q6PJU7_HUMAN (SEQ ID NO: 439):

A. An isolated chimeric polypeptide encoding for T86235_P7 (SEQ ID NO:472), comprising a first amino acid sequence being at least about 90% homologous to MTTRQATKDPLLRGVSPTPSKIPVRSQKRTPFPTVTSCAVDQENQDPRRWVQKPPLNIQRPLVDSAGPRPK ARHQAETSQRLVGISQPRNPLEELRPSPRGQNVGPGPPAQT corresponding to amino acids 1-112 of known protein(s) Q6PJU7_HUMAN (SEQ ID NO: 439), which also corresponds to amino acids 1-112 of T86235_P7 (SEQ ID NO:472), and a second amino acid sequence being at least about 70%, optionally at least 80%, preferably at least 85%, more preferably at least 90% and most preferably at least 95% homologous to a polypeptide having the sequence EAPGTIEFVADPAALATILSGEGVKSCHLGRQPSLAKRVLVRGSQGGTTQRVQGVRASAYLAPRTPTHRLD PARASCFSRLEGPGPRGRTLCPQRLQALISPSGPSFHPSTRPSFQELRRETAGSSRTSVSQASGLLLETPVQPA FSLPKGEREVVTHSDEGGVASLGLAQRVPLRENREMSHTRDSHDSHLMPSPAPVAQPLPGHVVPCPSPFGR AQRVPSPGPPTLTSYSVLRRLTVQPKTRFTPMPSTPRVQQAQWLRGVSPQSCSEDPALPWEQVAVRLFDQE SCIRSLEGSGKPPVATPSGPHSNRTPSLQEVKIQRIGILQQLLRQEVEGLVGGQCVPLNGGSSLDMVELQPL LTEISRTLNATEHNSGTSHLPGLLKHSGLPKPCLPEECGEPQPCPPAEPGPPEAFCRSEPEIPEPSLQEQLEVP EPYPPAEPRPLESCCRSEPEIPESSRQEQLEVPEPCPPAEPRPLESYCRIEPEIPESSRQEQLEVPEPCPPAEPGP LQPSTQGQSGPPGPCPRVELGASEPCTLEHRSLESSLPPCCSQWAPATTSLIFSSQHPLCASPPICSLQSLRPP AGQAGKELAGKECEHKRSSPHCELHTCPAPTPGNLMLPHLPPWPSLALPQEEGRGCTSSPVLLIGLAVGGG GGEDSTWWKYRTPDLPLNFPCPSGLSNLAPRTLALRERLKSCLTAIHCFHEARLDDECAFYTSRAPPSGPT RVCTNPVATLLEWQDALCFIPVGSAAPQGSP (SEQ ID NO: 517) corresponding to amino acids 113-868 of T86235_P7 (SEQ ID NO:472), wherein said first amino acid sequence and second amino acid sequence are contiguous and in a sequential order.

B. An isolated polypeptide encoding for an edge portion of T86235_P7 (SEQ ID NO:472), comprising an amino acid sequence being at least about 70%, optionally at least about 80%, preferably at least about 85%, more preferably at least about 90% and most preferably at least about 95% homologous to the sequence

(SEQ ID NO: 517)

EAPGTIEFVADPAALATILSEGVKSCHLGRQPSLAKRVLVRGSQGGTTQR
VQGVRASAYLAPRTPTHRLDPARASCFSRLEGPGPRGRTLCPQRLQALIS
PSGPSFHPSTRPSFQELRRETAGSSRTSVSQASGLLLETPVQPAFSLPKG
EREVVTHSDEGGVASLGLAQRVPLRENREMSHTRDSHDSHLMPSPAPVAQ
PLPGHVVPCPSPFGRAQRVPSPGPPTLTSYSVLRRTVQPKTRFTPMPSTP
RVQQAQWLRGVSPQSCSEDPALPWEQVAVRLFDQESCIRSLEGSGKPPVA
TPSGPHSNRTPSLQEVKIQRIGILQQLLRQEVEGLVGGQCVPLNGGSSLD
MVELQPLLTEISRTLNATEHNSGTSHLPGLLKHSGLPKPCLPEECGEPQP
CPPAEPGPPEAFCRSEPEIPEPSLQEQLEVPEPYPPAEPRPLESCCRSEP
EIPESSRQEQLEVPEPGPPAEPRPLESYCRIEPEIPESSRQEQLEVPEPC
PPAEPGPLQPSTQGQSGPPGPCPRVELGASEPCTLEHRSLESSLPPCCSQ
WAPATTSLIFSSQHPLCASPPICSLQSLRPPAGQAGKELAGKECEHKRSS
PHCELHTCPAPTPGNLMLPHLPPWPSLALPQEEGRGCTSSPVLLIGLAVG
GGFFEDSTWWKYRTPDLPLNFPCPSGLSNLAPRTLALRERLKSCTAIHCF
HEARLDDECARYTSRAPPSGPTRVCTNPVATLLEWQDALCFIPVGSAAPQ
GSP
of

(SEQ ID NO: 472)

T86235_P7.

The localization of the variant protein was determined according to results from a number of different software programs and analyses, including analyses from SignalP and other specialized programs. The variant protein is believed to be located intracellularly with regard to the cell.

Variant protein T86235_P7 (SEQ ID NO:472) also has the following non-silent SNPs (Single Nucleotide Polymorphisms) as listed in Table 162, (given according to their position(s) on the amino acid sequence, with the alternative amino acid(s) listed).

TABLE 162
Amino acid mutations

| SNP position(s) on amino acid | |
|---|---|
| sequence | Alternative amino acid(s) |
| 82 | L -> |
| 116 | G -> |
| 120 | F -> |
| 165 | Q -> |
| 242 | V -> A |
| 293 | M -> V |
| 333 | P -> L |
| 392 | A -> V |
| 559 | C -> |
| 562 | S -> G |
| 649 | L -> |
| 776 | Y -> S |
| 790 | G -> |

Variant protein T86235_P7 (SEQ ID NO:472) is encoded by the following transcript(s): T86235_T7 (SEQ ID NO:442), for which the coding portion starts at position 298 and ends at position 2901. The transcript also has the following SNPs as listed in Table 163 (given according to their position on the nucleotide sequence, with the alternative nucleic acid listed).

TABLE 163
Nucleic acid SNPs

| Polymorphism | SNP position(s) on nucleotide sequence |
|---|---|
| G -> | 240, 643, 792, 2666 |
| G -> A | 360, 1704, 2929 |
| A -> C | 439, 2624 |
| T -> | 541, 655, 1972 |
| C -> T | 909, 1272, 1295, 1374, 1472 |
| T -> C | 1022, 1890 |
| A -> G | 1174, 1509, 1632, 1981 |
| C -> | 2242 |

Variant protein T86235_P11 (SEQ ID NO:473) according to the present invention has an amino acid sequence encoded by transcript(s) T86235_T11 (SEQ ID NO:443). One or more alignments to one or more previously published protein sequences are given in the alignment table on the attached CDROM. A brief description of the relationship of the variant protein according to the present invention to each such aligned protein is as follows:

### 1. Comparison Report Between T86235_P111 (SEQ ID NO:473) and Known Protein(s) TAST_HUMAN (SEQ ID NO: 463):

A. An isolated chimeric polypeptide encoding for T86235_P111 (SEQ ID NO:473), comprising a first amino acid sequence being at least about 90% homologous to MTTRQATKDPLLRGVSPTPSKIPVRSQKRTPFPTVTSCAVDQENQDPRRWVQKPPLNIQRPLVDSAGPRPK ARHQAETSQRLVGISQPRNPLEELRPSPRGQNVGPGPPAQTEAPGTIEFVADPAALATILSGEGVKSCHLGR QPSLAKRVLVRGSQGGTTQRVQGVRASAYLAPRTPTHRLDPARASCFSRLEGPGPRGRTLCPQRLQALISP SGPSFHPST corresponding to amino acids 1-223 of known protein(s) TAST_HUMAN (SEQ ID NO: 463), which also corresponds to amino acids 1-223 of T86235_P11 (SEQ ID NO:473), a bridging amino acid R corresponding to amino acid 224 of T86235_P11 (SEQ ID NO:473), a second amino acid sequence being at least about 90% homologous to PSFQELRRETAGSSRTSVSQASGLLLETPVQPAFSLPKGEREVVTHSDEGGVASLGLAQRVPLRENREMSH TRDSHDSHLMPSPAPVAQPLPGHVVPCPSPFGRAQRVPSPGPPTLTSYSVLRRLTVQPKTRFTPMPSTPRVQ QAQWLRGVSPQSCSEDPALPWEQVAVRLFDQESCIRSLEGSGKPPVATPSGPHSNRTPSLQEVKIQRIGILQ QLLRQEVEGLVGGQCVPLNGGSSLDMVELQPLLTEISRTLNATEHNSGTSHLPGLLKHSGLPKPCLPEECG EPQPCPPAEPGPPEAFCRSEPEIPEPSLQEQLEVPEPYPPAEPRPLESCCRSEPEIPESSRQEQLE corresponding to amino acids 225-576 of known protein(s) TAST_HUMAN (SEQ ID NO: 463), which also corresponds to amino acids 225-576 of T86235_P11 (SEQ ID NO:473), a third amino acid sequence being at least about 90% homologous to EQLEVPEPCPPAEPGPLQPSTQGQSGPPGPCPRVELGASEPCTLEHRSLESSLPPCCSQWAPATTSLIFSSQHP LCASPPICSLQSLRPPAGQAGLSNLAPRTLALRERLKSCLTAIHCFHEARLDDECAFYTSRAPPSGPTRVCTN PVATLLEWQDAL corresponding to amino acids 606-764 of known protein(s) TAST_HUMAN (SEQ ID NO: 463), which also corresponds to amino acids 577-735 of T86235_P11 (SEQ ID NO:473), and a fourth amino acid sequence being at least about 70%, optionally at least 80%, preferably at least 85%, more preferably at least 90% and most preferably at least 95% homologous to a polypeptide having the sequence VRLQPTAQLWLHSEPDGRWGTGTGGHLGFFTERSAGRLGYSARLAEL (SEQ ID NO: 518) corresponding to amino acids 736-782 of T86235_P11 (SEQ ID NO:473), wherein said first amino acid sequence, bridging amino acid, second amino acid sequence, third amino acid sequence and fourth amino acid sequence are contiguous and in a sequential order.

B. An isolated chimeric polypeptide encoding for an edge portion of T86235_P11 (SEQ ID NO:473), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EE, having a structure as follows: a sequence starting from any of amino acid numbers 576−x to 576; and ending at any of amino acid numbers 577+((n−2)−x), in which x varies from 0 to n−2.

C. An isolated polypeptide encoding for an edge portion of T86235_P11 (SEQ ID NO:473), comprising an amino acid sequence being at least about 70%, optionally at least about 80%, preferably at least about 85%, more preferably at least about 90% and most preferably at least about 95% homologous to the sequence

(SEQ ID NO: 518)

VRLQPTAQLWLHSEPDGRWGTGTGGHLGFFTERSAGRLGYSARLAEL
of

(SEQ ID NO: 473)

T86235_P11.

### 2. Comparison Report Between T86235_P11 (SEQ ID NO:473) and Known Protein(s) Q8NSB2_HUMAN (SEQ ID NO: 467):

A. An isolated chimeric polypeptide encoding for T86235_P11 (SEQ ID NO:473), comprising a first amino acid sequence being at least about 90% homologous to

MTTRQATKDPLLRGVSPTPSKIPVRSQKRTPFPTVTSCAVDQENQDPRRW
VQKPPLNIQRPLVDSAGPRPKARHQAETSQRLVGISQPRNPLEELRPSPR
GQNVGPGPPAQTEAPGTIEFVDPAALATILSGEGVKSCHLGRQPSLAKRV
LVRGSQGGTTQRVQGVASAYLAPRTPTHRLDPARASCFSRLEGPGPRGRT
LCPQRLQALISPSGPSFHPSTRPSFQELRRETAGSSRTSVSQASGLLLET
PVQPAFSLPKGEREVVTHSDEGGVASLGLAQRVPLRENREMSHTRDSHDS
HLMPSPAPVAQPLPGHVVPCPSPFGRAQRVPSPGPPTLTSYSVLRRLTVQ
PKTRFTPMPSTPRVQQAQWLRGVSPQSCSEDPALPWEQVAVRLFDQESCI
RSLEGSGKPPVATPSGPHSNRTPSLQEVKIQRIGILQQLLRQEVEGLVGG
QCVPLNGGSSLDMVELQPLLTEISRTLNATEHNSGTSHLPGLLKHSGLPK
PCLPEECGEPQPCPPAEPGPPEAFCRSEPEIPEPSLQEQLEVPEPYPPAE
PRPLESCCRSEPEIPESSRQEQLE

corresponding to amino acids 1-576 of known protein(s) Q8N5B2_HUMAN (SEQ ID NO: 467), which also corresponds to amino acids 1-576 of T86235_P11 (SEQ ID NO:473), a second amino acid sequence being at least about 90% homologous to EQLEVPEPCPPAEPGPLQPSTQGQSGPPGPCPRVELGASEPCTLEHRSLESSLPPCCSQWAPATTSLIFSSQHP LCASPPICSLQSLRPPAGQAGLSNLAPRTLALRERLKSCLTAIHCFHEARLDDECAFYTSRAPPSGPTRVCTN PVATLLEWQDAL corresponding to amino acids 606-764 of known protein(s) Q8N5B2_HUMAN (SEQ ID NO: 467), which also corresponds to amino acids 577-735 of T86235_P11 (SEQ ID NO:473), and a third amino acid sequence being at least about 70%, optionally at least 80%, preferably at least 85%, more preferably at least 90% and most preferably at least 95% homologous to a polypeptide having the sequence VRLQPTAQLWLHSEPDGRW GTGTGGHLGFFTERSAGRLGYSARLAEL (SEQ ID NO: 518) corresponding to amino acids 736-782 of T86235_P11 (SEQ ID NO:473), wherein said first amino acid sequence, second amino acid sequence and third amino acid sequence are contiguous and in a sequential order.

B. An isolated chimeric polypeptide encoding for an edge portion of T86235_P11 (SEQ ID NO:473), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EE, having a structure as follows: a sequence starting from any of amino acid numbers 576−x to 576; and ending at any of amino acid numbers 577+((n−2)−x), in which x varies from 0 to n−2.

C. An isolated polypeptide encoding for an edge portion of T86235_P11 (SEQ ID NO:473), comprising an amino acid sequence being at least about 70%, optionally at least about 80%, preferably at least about 85%, more preferably at least about 90% and most preferably at least about 95% homologous to the sequence

(SEQ ID NO: 518)

VRLQPTAQLWLHSEPDGRWGTGTGGHLGFFTERSAGRLGYSARLAEL
of

(SEQ ID NO: 473)

T86235_P11.

### 3. Comparison Report Between T86235_P11 (SEQ ID NO:473) and Known Protein(s) Q6PJU7_HUMAN (SEQ ID NO: 439):

A. An isolated chimeric polypeptide encoding for T86235_P11 (SEQ ID NO:473), comprising a first amino acid sequence being at least about 90% homologous to MTTRQATKDPLLRGVSPTPSKIPVRSQKRTPFPTVTSCAVDQENQDPRRWVQKPPLNIQRPLVDSAGPRPK ARHQAETSQRLVGISQPRNPLEELRPSPRGQNVGPGPPAQT corresponding to amino acids 1-112 of known protein(s) Q6PJU7_HUMAN (SEQ ID NO: 439), which also corresponds to amino acids 1-112 of T86235_P11 (SEQ ID NO:473), and a second amino acid sequence being at least about 70%, optionally at least 80%, preferably at least 85%, more preferably at least 90% and most preferably at least 95% homologous to a polypeptide having the sequence EAPGTIEFVADPAALATILSGEGVKSCHLGRQPSLAKRVLVRGSQGGTTQRVQGVRASAYLAPRTPPTRLD PARASCFSRLEGPGPRGRTLCPQRLQALISPSGPSFHPSTRPSFQELRRETAGSSRTSVSQASGLLLETPVQPA FSLPKGEREVVTHSDEGGVASLGLAQRVPLRENREMSHTRDSHDSHLMPSPAPVAQPLPGHVVPCPSPFGR AQRVPSPGPPTLTSYSVLRRLTVQPKTRFTPNPSTPRVQQAQWLRGVSPQSCSEDPALPWEQVAVRLFDQE SCIRSLEGSGKPPVATPSGPHSNRTPSLQEVKIQRIGILQQLLRQEVEGLVGGQCVPLNGGSSLDMVELQPL LTEISRTLNATEHNSGTSHLPGLLKHSGLPKPCLPEECGEPQPCPPAEPGPPEAFCRSEPEIPEPSLQEQLEVP EPYPPAEPRPLESCCRSEPEIPESSRQEQLEEQLEVPEPCPPAEPGPLQPSTQGQSGPPGPCPRVELGASEPCT LEHRSLESSLPPCCSQWAPATTSLIFSSQHPLCASPPICSLQSLRPPAGQAGLSNLAPRTLALRERLKSCLTAI HCFHEARLDDECAFYTSRAPPSGPTRVCTNPVATLLEWQDALVRLQPTAQLWLHSEPDGRWGTGTGGHL GFFTERSAGRLGYSARLAEL (SEQ ID NO: 519) corresponding to amino acids 113-782 of T86235_P11 (SEQ ID NO:473), wherein said first amino acid sequence and second amino acid sequence are contiguous and in a sequential order.

B. An isolated polypeptide encoding for an edge portion of T86235_P11 (SEQ ID NO:473), comprising an amino acid sequence being at least about 70%, optionally at least about 80%, preferably at least about 85%, more preferably at least about 90% and most preferably at least about 95% homologous to the sequence

(SEQ ID NO: 519)

EAPGTIEFVADPAALATILSGEGVKSCHLGRQPSLAKRVLVRGSQGGTTQ
RVQGVRASAYLAPRTPTHRLDPARASCFSRLEGPGPRGRTLCPQRIQALI
SPSGPSFHPSTRPSFQELRRETAGSSRTSVSQASGLLLETPVQPAFSLPK
GEREVVTHSDEGGVASLGLAQRVPLRENREMSHTRDSHDSHLMPSPAPVA
QPLPGHVVPCPSPFGRAQRVPSPGPPTLTSYSVLRRLTVQPKTRFTPMPS
TPRVQQAQWLRGVSPQSCSEDPALPWEQVAVRLFDQESCIRSLEGSGKPP
VATPSGPHSNRTPSLQEVKIQRIGILQQLLRQEVEGLVGGQCVPLNGGSS
LDMVELQPLLTEISRTLNATEHNSGTSHLPGLLKHSGLPKPCLPEECGEP
QPCPPAEPGPPEAFCRSEPEIPEPSLQEQLEVPEPYPPAEPRPLESCCRS
EPEIPESSRQEQLEEQLEVPEPCPPAEPGPLQPSTQGQSGPPGPCPRVEL
GASEPCTLEHRSLESSLPPCCSQWAPATTSLIFSSQHPLCASPPICSLQS
LRPPAGQAGLSNLAPRTLALRERLKSCLTAIHCFHEARLDDECAFYTSRA
PPSGPTRVCTNPVATLLEWQDALVRLQPTAQLWLHSEPDGRWGTGTGGHL
GFFTERSAGRLGYSARLAEL
of

(SEQ ID NO: 473)

T86235_P11.

The localization of the variant protein was determined according to results from a number of different software programs and analyses, including analyses from SignalP and other specialized programs. The variant protein is believed to be located intracellularly with regard to the cell.

Variant protein T86235_P11 (SEQ ID NO:473) also has the following non-silent SNPs (Single Nucleotide Polymorphisms) as listed in Table 164, (given according to their position(s) on the amino acid sequence, with the alternative amino acid(s) listed).

TABLE 164
Amino acid mutations

| SNP position(s) on amino acid | |
|---|---|
| sequence | Alternative amino acid(s) |
| 82 | L -> |
| 116 | G -> |
| 120 | F -> |
| 165 | Q -> |
| 242 | V -> A |
| 293 | M -> V |
| 333 | P -> L |
| 392 | A -> V |
| 559 | C -> |
| 562 | S -> G |
| 620 | L -> |
| 671 | G -> |

Variant protein T86235_P11 (SEQ ID NO:473) is encoded by the following transcript(s): T86235_T11 (SEQ ID NO:443), for which the coding portion starts at position 298 and ends at position 2643. The transcript also has the following SNPs as listed in Table 165 (given according to their position on the nucleotide sequence, with the alternative nucleic acid listed).

TABLE 165
Nucleic acid SNPs

| Polymorphism | SNP position(s) on nucleotide sequence |
|---|---|
| G -> | 240, 643, 792, 2309 |
| G -> A | 360, 1704, 2737 |
| A -> C | 439 |
| T -> | 541, 655, 1972 |
| C -> T | 909, 1272, 1295, 1374, 1472 |
| T -> C | 1022, 1890, 2652 |
| A -> G | 1174, 1509, 1632, 1981 |
| C -> | 2155 |

Variant protein T86235_P15 (SEQ ID NO:474) according to the present invention has an amino acid sequence encoded by transcript(s) T86235_T18 (SEQ ID NO:446). One or more alignments to one or more previously published protein sequences are given in the alignment table on the attached CDROM. A brief description of the relationship of the variant protein according to the present invention to each such aligned protein is as follows:

### 1. Comparison Report Between T86235_P15 (SEQ ID NO:474) and Known Protein(s) TAST_HUMAN (SEQ ID NO: 463):

A. An isolated chimeric polypeptide encoding for T86235_P15 (SEQ ID NO:474), comprising a first amino acid sequence being at least about 90% homologous to MTTRQATKDPLLRGVSPTPSKIPVRSQKRTPFPTVTSCAVDQENQDPRRWVQKPPLNIQRPLVDSAGPRPK ARHQAETSQRLVGISQPRNPLEELRPSPRGQNVGPGPPAQTEAPGTIEFVADPAALATILSGEGVKSCHLGR QPSLAKRVLVRGSQGGTTQRVQGVRASAYLAPRTPTHRLDPARASCFSRLEGPGPRGRTLCPQRLQALISP SGPSFHPST corresponding to amino acids 1-223 of known protein(s) TAST_HUMAN (SEQ ID NO: 463), which also corresponds to amino acids 1-223 of T86235_P15 (SEQ ID NO:474), a bridging amino acid R corresponding to amino acid 224 of T86235_P15 (SEQ ID NO:474), a second amino acid sequence being at least about 90% homologous to PSFQELRRETAGSSRTSVSQASGLLLETPVQPAFSLPKGEREVVTHSDEGGVASLGLAQRVPLRENREMSH TRDSHDSHLMPSPAPVAQPLPGHVVPCPSPFGRAQRVPSPGPPTLTSYSVLRRLTVQPKTRFTPMPSTPRVQ QAQWLRGVSPQSCSEDPALPWEQVAVRLFDQESCIRSLEGSGKPPVATPSGPHSNRTPSLQEVKIQRIGILQ QLLRQEVEGLVGGQCVPLNGGSSLDMVELQPLLTEISRTLNATEHNSGTSHLPGLLKHSGLPKPCLPEECG EPQPCPPAEPGPPEAFCRSEPEIPEPSLQEQLEVPEPYPPAEPRPLESCCRSEPEIPESSRQEQLE corresponding to amino acids 225-576 of known protein(s) TAST_HUMAN (SEQ ID NO: 463), which also corresponds to amino acids 225-576 of T86235_P15 (SEQ ID NO:474), a third amino acid sequence being at least about 90% homologous to EQLEVPEPCPPAEPGPLQPSTQGQSGPPGPCPRVELGASEPCTLEHRSLESSLPPCCSQWAPATTSLIFSSQHP LCASPPICSLQSLRPPAGQA corresponding to amino acids 606-699 of known protein(s) TAST_HUMAN (SEQ ID NO: 463) and which also corresponds to amino acids 577-670 of T86235_P15 (SEQ ID NO:474), a fourth amino acid sequence being at least about 70%, optionally at least 80%, preferably at least 85%, more preferably at least 90% and most preferably at least 95% homologous to a polypeptide having the sequence GKELAGKECEHKRSSPHCELHTCPAPTPGNLMLPHLPPWPSLALPQEEGRGCTSSPVLLIGLAVGGGGGED STWWKYRTPDLPLNFPCPS (SEQ ID NO: 514) corresponding to amino acids 671-760 of T86235_P15 (SEQ ID NO:474), a fifth amino acid sequence being at least about 90% homologous to GLSNLAPRTLALRERLKSCLTAIHCFHEARLDDECAFYTSRAPPSGPTRVCTNPVATLLEWQDAL corresponding to amino acids 700-764 of known protein(s) TAST_HUMAN (SEQ ID NO: 463), which also corresponds to amino acids 761-825 of T86235_P15 (SEQ ID NO:474), and a sixth amino acid sequence being at least about 70%, optionally at least 80%, preferably at least 85%, more preferably at least 90% and most preferably at least 95% homologous to a polypeptide having the sequence VRLQPTAQLWLHSEPDGRWGTGTGGHLGFFTERSAGRLGYSARLAEL (SEQ ID NO: 518) corresponding to amino acids 826-872 of T86235_P15 (SEQ ID NO:474), wherein said first amino acid sequence, bridging amino acid, second amino acid sequence, third amino acid sequence, fourth amino acid sequence, fifth amino acid sequence and sixth amino acid sequence are contiguous and in a sequential order.

B. An isolated chimeric polypeptide encoding for an edge portion of T86235_P15 (SEQ ID NO:474), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EE, having a structure as follows: a sequence starting from any of amino acid numbers 576−x to 576; and ending at any of amino acid numbers 577+((n−2)−x), in which x varies from 0 to n−2.

C. An isolated polypeptide encoding for an edge portion of T86235_P15 (SEQ ID NO:474), comprising an amino acid sequence being at least about 70%, optionally at least about 80%, preferably at least about 85%, more preferably at least about 90% and most preferably at least about 95% homologous to the sequence

(SEQ ID NO: 514)

GKELAGKECEHKRSSPHCELHTCPAPTPGNLMLPHLPPWPSLALPQEEGR
GCTSSPVLLIGLAVGGGGGEDSTWWKYRTPDLPLNFPCPS
of

(SEQ ID NO: 474)

T86235_P15.

D. An isolated polypeptide encoding for an edge portion of T86235_P15 (SEQ ID NO:474), comprising an amino acid sequence being at least about 70%, optionally at least about 80%, preferably at least about 85%, more preferably at least about 90% and most preferably at least about 95% homologous to the sequence

(SEQ ID NO: 518)

VRLQPTAQLWLHSEPDGRWGTGTGGHLGFFTERSAGRLGYSARLAEL
of

(SEQ ID NO: 474)

T86235_P15.

### 2. Comparison Report Between T86235_P15 (SEQ ID NO:474) and Known Protein(s) Q8N5B2_HUMAN (SEQ ID NO: 467):

A. An isolated chimeric polypeptide encoding for T86235_P15 (SEQ ID NO:474), comprising a first amino acid sequence being at least about 90% homologous to MTTRQATKDPLLRGVSPTPSKIPVRSQKRTPFPTVTSCAVDQENQDPRRWVQKPPLNIQRPLVDSAGPRPK ARHQAETSQRLVGISQPRNPLEELRPSPRGQNVGPGPPAQTEAPGTIEFVADPAALATILSGEGVKSCHLGR QPSLAKRVLVRGSQGGTTQRVQGVRASAYLAPRTPTBRLDPARASCFSRLEGPGPRGRTLCPQRLQALISP SGPSFHPSTRPSFQELRRETAGSSRTSVSQASGLLLETPVQPAFSLPKGEREVVTHSDEGGVASLGLAQRVP LRENREMSHTRDSHDSHLMPSPAPVAQPLPGHVVPCPSPFGRAQRVPSPGPPTLTSYSVLRRLTVQPKTRFT PMPSTPRVQQAQWLRGVSPQSCSEDPALPWEQVAVRLFDQESCIRSLEGSGKPPVATPSGPHSNRTPSLQE VKIQRIGILQQLLRQEVEGLVGGQCVPLNGGSSLDMVELQPLLTEISRTLNATEHNSGTSHLPGLLKHSGLP KPCLPEECGEPQPCPPAEPGPPEAFCRSEPEIPEPSLQEQLEVPEPYPPAEPRPLESCCRSEPEIPESSRQEQLE corresponding to amino acids 1-576 of known protein(s) Q8N5B2_HUMAN (SEQ ID NO: 467), which also corresponds to amino acids 1-576 of T86235_P15 (SEQ ID NO:474), a second amino acid sequence being at least about 90% homologous to EQLEVPEPCPPAEPGPLQPSTQGQSGPPGPCPRVELGASEPCTLEHRSLESSLPPCCSQWAPATTSLIFSSQHP LCASPPICSLQSLRPPAGQA corresponding to amino acids 606-699 of known protein(s) Q8N5B2_HUMAN (SEQ ID NO: 467), which also corresponds to amino acids 577-670 of T86235_P15 (SEQ ID NO:474), a third amino acid sequence being at least about 70%, optionally at least 80%, preferably at least 85%, more preferably at least 90% and most preferably at least 95% homologous to a polypeptide having the sequence GKELAGKECEHKRSSPHCELHTCPAPTPGNLMLPHLPPWPSLALPQEEGRGCTSSPVLLIGLAVGGGGGED STWWKYRTPDLPLNFPCPS (SEQ ID NO: 514) corresponding to amino acids 671-760 of T86235_P15 (SEQ ID NO:474), a fourth amino acid sequence being at least about 90% homologous to GLSNLAPRTLALRERLKSCLTAIHCFHEARLDDECAFYTSRAPPSGPTRVCTNPVATLLEWQDAL corresponding to amino acids 700-764 of known protein(s) Q8N5B2_HUMAN (SEQ ID NO: 467), which also corresponds to amino acids 761-825 of T86235_P15 (SEQ ID NO:474), and a fifth amino acid sequence being at least about 70%, optionally at least 80%, preferably at least 85%, more preferably at least 90% and most preferably at least 95% homologous to a polypeptide having the sequence VRLQPTAQLWLHSEPDGRWGTGTGGHLGFFTERSAGRLGYSARLAEL (SEQ ID NO: 518) corresponding to amino acids 826-872 of T86235_P15 (SEQ ID NO:474), wherein said first amino acid sequence, second amino acid sequence, third amino acid sequence, fourth amino acid sequence and fifth amino acid sequence are contiguous and in a sequential order.

### 3. Comparison Report Between T86235_P15 (SEQ ID NO:474) and Known Protein(s) Q6PJU7_HUMAN (SEQ ID NO: 439):

A. An isolated chimeric polypeptide encoding for T86235_P15 (SEQ ID NO:474), comprising a first amino acid sequence being at least about 90% homologous to MTTRQATKDPLLRGVSPTPSKIPVRSQKRTPFPTVTSCAVDQENQDPRRWVQKPPLNIQRPLVDSAGPRPK ARHQAETSQRLVGISQPRNPLEELRPSPRGQNVGPGPPAQT corresponding to amino acids 1-112 of known protein(s) Q6PJU7_HUMAN (SEQ ID NO: 439), which also corresponds to amino acids 1-112 of T86235_P15 (SEQ ID NO:474), and a second amino acid sequence being at least about 70%, optionally at least 80%, preferably at least 85%, more preferably at least 90% and most preferably at least 95% homologous to a polypeptide having the sequence EAPGTIEFVADPAALATILSGEGVKSCHLGRQPSLAKRVLVRGSQGGTTQRVQGVRASAYLAPRTPTHRLD PARASCFSRLEGPGPRGRTLCPQRLQALISPSGPSFHPSTRPSFQELRRETAGSSRTSVSQASGLLLETPVQPA FSLPKGEREVVTHSDEGGVASLGLAQRVPLRENREMSHTRDSHDSHLMPSPAPVAQPLPGHVVPCPSPFGR AQRVPSPGPPTLTSYSVLRRLTVQPKTRFTPMPSTPRVQQAQWLRGVSPQSCSEDPALPWEQVAVRLFDQE SCIRSLEGSGKPPVATPSGPHSNRTPSLQEVKIQRIGILQQLLRQEVEGLVGGQCVPLNGGSSLDMVELQPL LTEISRTLNATEHNSGTSHLPGLLKHSGLPKPCLPEECGEPQPCPPAEPGPPEAFCRSEPEIPEPSLQEQLEVP EPYPPAEPRPLESCCRSEPEIPESSRQEQLEEQLEVPEPCPPAEPGPLQPSTQGQSGPPGPCPRVELGASEPCT LEHRSLESSLPPCCSQWAPATTSLIFSSQHPLCASPPICSLQSLRPPAGQAGKELAGKECEHKRSSPHCELHT CPAPTPGNLMLPHLPPWPSLALPQEEGRGCTSSPVLLIGLAVGGGGGEDSTWWKYRTPDLPLNFPCPSGLS NLAPRTLALRERLKSCLTAIHCFHEARLDDECAFYTSRAPPSGPTRVCTNPVATLLEWQDALVRLQPTAQL WLHSEPDGRWGTGTGGHLGFFTERSAGRLGYSARLAEL (SEQ ID NO: 522) corresponding to amino acids 113-872 of T86235_P15 (SEQ ID NO:474), wherein said first amino acid sequence and second amino acid sequence are contiguous and in a sequential order.

B. An isolated polypeptide encoding for an edge portion of T86235_P15 (SEQ ID NO:474), comprising an amino acid sequence being at least about 70%, optionally at least about 80%, preferably at least about 85%, more preferably at least about 90% and most preferably at least about 95% homologous to the sequence

(SEQ ID NO: 522)

EAPGTIEFVADPAALATILSGEGVKSCHLGRQPSLAKRVLVRGSQGGTTQ
RVQGVRASAYPRTPTHRLDPARASCFSRLEGPGPRGRTLCPQRLQALISP
SGPSFHPSTRPSFQELRRETAGSSRTSVSQASGLLLETPVQPAFSLPKGE
REVVTHSDEGGVASLGLAQRVPLRENEMSHTDSHDSHLMPSPAPVAQPLP
GHVVPCPSPFGRAQRVPSPGPPTLTSYSVLRRLTVQPKTRFTPMPSTPRV
QQAQWLRGVSPQSCSEDPALPWEQVAVRLFDQESCIRSLEGSGKPPVATP
SGPHSNRTPSLQEVKIQRIGILQQLLRQEVEGLVGGQCVPLNGGSSLDMV
ELQPLLTEISRTLNATEHNSGTSHLPGLLKHSGLPKPCLPEECGEPQPCP
PAEPGPPEAFCRSEPEIPEPSLQEQLEVPEPYPPAEPRPLESCCRSEPEI
PESSRQEQLEEQLEVPEPCPPAEPGPLQPSTQGQSGPPGPCPRVELGASE
PCTLEHRSLESSLPPCCSQWAPATTSLIFSSQHPLCASPPICSLQSLRPP
AGQAGKELAGKECEHKRSSPHCELHTCPAPTPGNLMLPHLPPWPSLALPQ
EEGRGCTSSPVLLIGLAVGGGGGEDSTWWKYRTPDLPLNFPCPSGLSNLA
PRTLALRERLKSCLTAIHCFHEARLDDECAFYTSRAPPSGPTRVCTNPVA
TLLEWQDALVRLQPTAQLWLHSEPDGRWGTGTGGHLGFFTERSAGRLGYS
ARLAEL
of

(SEQ ID NO: 474)

T86235_P15.

The localization of the variant protein was determined according to results from a number of different software programs and analyses, including analyses from SignalP and other specialized programs. The variant protein is believed to be located intracellularly with regard to the cell.

Variant protein T86235_P15 (SEQ ID NO:474) also has the following non-silent SNPs (Single Nucleotide Polymorphisms) as listed in Table 166, (given according to their position(s) on the amino acid sequence, with the alternative amino acid(s) listed).

TABLE 166
Amino acid mutations

| SNP position(s) on amino acid | |
|---|---|
| sequence | Alternative amino acid(s) |
| 82 | L -> |
| 116 | G -> |
| 120 | F -> |
| 165 | Q -> |
| 242 | V -> A |
| 293 | M -> V |
| 333 | P -> L |
| 392 | A -> V |
| 559 | C -> |
| 562 | S -> G |
| 620 | L -> |
| 747 | Y -> S |
| 761 | G -> |

Variant protein T86235_P15 (SEQ ID NO:474) is encoded by the following transcript(s): T86235_T18 (SEQ ID NO:446), for which the coding portion starts at position 298 and ends at position 2913. The transcript also has the following SNPs as listed in Table 167 (given according to their position on the nucleotide sequence, with the alternative nucleic acid listed).

TABLE 167
Nucleic acid SNPs

| Polymorphism | SNP position(s) on nucleotide sequence |
|---|---|
| G -> | 240, 643, 792, 2579 |
| G -> A | 360, 1704, 3007 |
| A -> C | 439, 2537 |
| T -> | 541, 655, 1972 |
| C -> T | 909, 1272, 1295, 1374, 1472 |
| T -> C | 1022, 1890, 2922 |
| A -> G | 1174, 1509, 1632, 1981 |
| C -> | 2155 |

Variant protein T86235_P21 (SEQ ID NO:475) according to the present invention has an amino acid sequence encoded by transcript(s) T86235_T26 (SEQ ID NO:447). One or more alignments to one or more previously published protein sequences are given in the alignment table on the attached CDROM. A brief description of the relationship of the variant protein according to the present invention to each such aligned protein is as follows:

### 1. Comparison Report Between T86235_P21 (SEQ ID NO:475) and Known Protein(s) TAST_HUMAN (SEQ ID NO: 463):

A. An isolated chimeric polypeptide encoding for T86235_P21 (SEQ ID NO:475), comprising a first amino acid sequence being at least about 70%, optionally at least 80%, preferably at least 85%, more preferably at least 90% and most preferably at least 95%, homologous to a polypeptide having the sequence KKKKKISQIQLNESLAHLVPQSSYARGPIPLSLPPSFSFSSFLA (SEQ ID NO: 523) corresponding to amino acids 1-44 of T86235_P21 (SEQ ID NO:475), a second amino acid sequence being at least about 90% homologous to EAPGTIEFVADPAALATILSGEGVKSCHLGRQPSLAKRVLVRGSQGGTTQRVQGVRASAYLAPRTPTBRLD PARASCFSRLEGPGPRGRTLCPQRLQALISPSGPSFHPST corresponding to amino acids 113-223 of known protein(s) TAST_HUMAN (SEQ ID NO: 463), which also corresponds to amino acids 45-155 of T86235_P21 (SEQ ID NO:475), a bridging amino acid R corresponding to amino acid 156 of T86235_P21 (SEQ ID NO:475), a third amino acid sequence being at least about 90% homologous to PSFQELRRETAGSSRTSVSQASGLLLETPVQPAFSLPKGEREVVTHSDEGGVASLGLAQRVPLRENREMSH TRDSHDSHLMPSPAPVAQPLPGHVVPCPSPFGRAQRVPSPGPPTLTSYSVLRRLTVQPKTRFTPMPSTPRVQ QAQWLRGVSPQSCSEDPALPWEQVAVRLFDQESCIRSLEGSGKPPVATPSGPHSNRTPSLQEVKIQRIGILQ QLLRQEVEGLVGGQCVPLNGGSSLDMVELQPLLTEISRTLNATEHNSGTSHLPGLLKHSGLPKPCLPEECG EPQPCPPAEPGPPEAFCRSEPEIPEPSLQEQLEVPEPYPPAEPRPLESCCRSEPEIPESSRQEQLE corresponding to amino acids 225-576 of known protein(s) TAST_HUMAN (SEQ ID NO: 463), which also corresponds to amino acids 157-508 of T86235_P21 (SEQ ID NO:475), and a fourth amino acid sequence being at least about 90% homologous to EQLEVPEPCPPAEPGPLQPSTQGQSGPPGPCPRVELGASEPCTLEHRSLESSLPPCCSQWAPATTSLIFSSQHP LCASPPICSLQSLRPPAGQAGLSNLAPRTLALRERLKSCLTAIHCFHEARLDDECAFYTSRAPPSGPTRVCTN PVATLLEWQDALCFIPVGSAAPQGSP corresponding to amino acids 606-778 of known protein(s) TAST_HUMAN (SEQ ID NO: 463), which also corresponds to amino acids 509-681 of T86235_P21 (SEQ ID NO:475), wherein said first amino acid sequence, second amino acid sequence, bridging amino acid, third amino acid sequence and fourth amino acid sequence are contiguous and in a sequential order.

B. An isolated polypeptide encoding for a head of T86235_P21 (SEQ ID NO:475), comprising a polypeptide being at least 70%, optionally at least about 80%, preferably at least about 85%, more preferably at least about 90% and most preferably at least about 95% homologous to the sequence KKKKKISQIQLNESLAHLVPQSSYARGPIPLSLPPSFSFSSFLA (SEQ ID NO: 523) of T86235_P21 (SEQ ID NO:475).

C. An isolated chimeric polypeptide encoding for an edge portion of T86235_P21 (SEQ ID NO:475), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EE, having a structure as follows: a sequence starting from any of amino acid numbers 508−x to 508; and ending at any of amino acid numbers 509+((n−2)−x), in which x varies from 0 to n−2.

### 2. Comparison Report Between T86235_P21 (SEQ ID NO:475) and Known Protein(s) Q8N5B2_HUMAN (SEQ ID NO: 467):

A. An isolated chimeric polypeptide encoding for T86235_P21 (SEQ ID NO:475), comprising a first amino acid sequence being at least about 70%, optionally at least 80%, preferably at least 85%, more preferably at least 90% and most preferably at least 95%, homologous to a polypeptide having the sequence KKKKKISQIQLNESLAHLVPQSSYARGPIPLSLPPSFSFSSFLA (SEQ ID NO: 523) corresponding to amino acids 1-44 of T86235_P21 (SEQ ID NO:475), a second amino acid sequence being at least about 90% homologous to EAPGTIEFVADPAALATILSGEGVKSCHLGRQPSLAKRVLVRGSQGGTTQRVQGVRASAYLAPRTPTHRLD PARASCFSRLEGPGPRGRTLCPQRLQALISPSGPSFHPSTRPSFQELRRETAGSSRTSVSQASGLLLETPVQPA FSLPKGEREVVTHSDEGGVASLGLAQRVPLRENREMSHTRDSHDSHLMPSPAPVAQPLPGHVVPCPSPFGR AQRVPSPGPPTLTSYSVLRRLTVQPKTRFTPMPSTPRVQQAQWLRGVSPQSCSEDPALPWEQVAVRLFDQE SCIRSLEGSGKPPVATPSGPHSNRTPSLQEVKIQRIGILQQLLRQEVEGLVGGQCVPLNGGSSLDMVELQPL LTEISRTLNATEHNSGTSHLPGLLKHSGLPKPCLPEECGEPQPCPPAEPGPPEAFCRSEPEIPEPSLQEQLEVP EPYPPAEPRPLESCCRSEPEIPESSRQEQLE corresponding to amino acids 113-576 of known protein(s) Q8N5B2_HUMAN (SEQ ID NO: 467), which also corresponds to amino acids 45-508 of T86235_P21 (SEQ ID NO:475), and a third amino acid sequence being at least about 90% homologous to EQLEVPEPCPPAEPGPLQPSTQGQSGPPGPCPRVELGASEPCTLEHRSLESSLPPCCSQWAPATTSLIFSSQHP LCASPPICSLQSLRPPAGQAGLSNLAPRTLALRERLKSCLTAIHCFHEARLDDECAFYTSRAPPSGPTRVCTN PVATLLEWQDALCFIPVGSAAPQGSP corresponding to amino acids 606-778 of known protein(s) Q8N5B2_HUMAN (SEQ ID NO: 467), which also corresponds to amino acids 509-681 of T86235_P21 (SEQ ID NO:475), wherein said first amino acid sequence, second amino acid sequence and third amino acid sequence are contiguous and in a sequential order.

The localization of the variant protein was determined according to results from a number of different software programs and analyses, including analyses from SignalP and other specialized programs. The variant protein is believed to be located intracellularly with regard to the cell.

Variant protein T86235_P21 (SEQ ID NO:475) also has the following non-silent SNPs (Single Nucleotide Polymorphisms) as listed in Table 168, (given according to their position(s) on the amino acid sequence, with the alternative amino acid(s) listed).

TABLE 168
Amino acid mutations

| SNP position(s) on amino acid | |
|---|---|
| sequence | Alternative amino acid(s) |
| 48 | G -> |
| 52 | F -> |
| 97 | Q -> |
| 174 | V -> A |
| 225 | M -> V |
| 265 | P -> L |
| 324 | A -> V |
| 491 | C -> |
| 494 | S -> G |
| 552 | L -> |
| 603 | G -> |

Variant protein T86235_P21 (SEQ ID NO:475) is encoded by the following transcript(s): T86235_T26 (SEQ ID NO:447), for which the coding portion starts at position 2 and ends at position 2044. The transcript also has the following SNPs as listed in Table 169 (given according to their position on the nucleotide sequence, with the alternative nucleic acid listed; T86235_P21 (SEQ ID NO:475)).

TABLE 169
Nucleic acid SNPs

| Polymorphism | SNP position(s) on nucleotide sequence |
|---|---|
| G -> | 143, 292, 1809 |
| T -> | 155, 1472 |
| C -> T | 409, 772, 795, 874, 972 |
| T -> C | 522, 1390 |
| A -> G | 674, 1009, 1132, 1481 |
| G -> A | 1204, 2072 |
| C -> | 1655 |

Variant protein T86235_P26 (SEQ ID NO:476) according to the present invention has an amino acid sequence encoded by transcript(s) T86235_T32 (SEQ ID NO:448). One or more alignments to one or more previously published protein sequences are given in the alignment table on the attached CDROM. A brief description of the relationship of the variant protein according to the present invention to each such aligned protein is as follows:

### 1. Comparison Report Between T86235_P26 (SEQ ID NO:476) and Known Protein(s) TAST_HUMAN (SEQ ID NO: 463):

A. An isolated chimeric polypeptide encoding for T86235_P26 (SEQ ID NO:476), comprising a first amino acid sequence being at least about 90% homologous to MVELQPLLTEISRTLNATEHNSGTSHLPGLLKHSGLPKPCLPEECGEPQPCPPAEPGPPEAFCRSEPEIPEPSL QEQLEVPEPYPPAEPRPLESCCRSEPEIPESSRQEQLE corresponding to amino acids 465-576 of known protein(s) TAST_HUMAN (SEQ ID NO: 463), which also corresponds to amino acids 1-112 of T86235_P26 (SEQ ID NO:476), and a second amino acid sequence being at least about 90% homologous to EQLEVPEPCPPAEPGPLQPSTQGQSGPPGPCPRVELGASEPCTLEHRSLESSLPPCCSQWAPATTSLIFSSQHP LCASPPICSLQSLRPPAGQAGLSNLAPRTLALRERLKSCLTAIHCFHEARLDDECAFYTSRAPPSGPTRVCTN PVATLLEWQDALCFIPVGSAAPQGSP corresponding to amino acids 606-778 of known protein(s) TAST_HUMAN (SEQ ID NO: 463), which also corresponds to amino acids 113-285 of T86235_P26 (SEQ ID NO:476), wherein said, first amino acid sequence and second amino acid sequence are contiguous and in a sequential order.

B. An isolated chimeric polypeptide encoding for an edge portion of T86235_P26 (SEQ ID NO:476), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EE, having a structure as follows: a sequence starting from any of amino acid numbers 112−x to 112; and ending at any of amino acid numbers 113+((n−2)−x), in which x varies from 0 to n−2.

### 2. Comparison Report Between T86235_P26 (SEQ ID NO:476) and Known Protein(s) Q8N5B2_HUMAN (SEQ ID NO: 467):

A. An isolated chimeric polypeptide encoding for T86235_P26 (SEQ ID NO:476), comprising a first amino acid sequence being at least about 90% homologous to MVELQPLLTEISRTLNATEHNSGTSHLPGLLKHSGLPKPCLPEECGEPQPCPPAEPGPPEAFCRSEPEIPEPSL QEQLEVPEPYPPAEPRPLESCCRSEPEIPESSRQEQLE corresponding to amino acids 465-576 of known protein(s) Q8N5B2_HUMAN (SEQ ID NO: 467), which also corresponds to amino acids 1-112 of T86235_P26 (SEQ ID NO:476), and a second amino acid sequence being at least about 90% homologous to EQLEVPEPCPPAEPGPLQPSTQGQSGPPGPCPRVELGASEPCTLEHRSLESSLPPCCSQWAPATTSLIFSSQHP LCASPPICSLQSLRPPAGQAGLSNLAPRTLALRERLKSCLTAIHCFHEARLDDECAFYTSRAPPSGPTRVCTN PVATLLEWQDALCFIPVGSAAPQGSP corresponding to amino acids 606-778 of known protein(s) Q8N5B2_HUMAN (SEQ ID NO: 467), which also corresponds to amino acids 113-285 of T86235_P26 (SEQ ID NO:476), wherein said, first amino acid sequence and second amino acid sequence are contiguous and in a sequential order.

The localization of the variant protein was determined according to results from a number of different software programs and analyses, including analyses from SignalP and other specialized programs. The variant protein is believed to be located as follows with regard to the cell: intracellularly.

Variant protein T86235_P26 (SEQ ID NO:476) also has the following non-silent SNPs (Single Nucleotide Polymorphisms) as listed in Table 170, (given according to their position(s) on the amino acid sequence, with the alternative amino acid(s) listed).

TABLE 170
Amino acid mutations

| SNP position(s) on amino acid | |
|---|---|
| sequence | Alternative amino acid(s) |
| 95 | C -> |
| 98 | S -> G |
| 156 | L -> |
| 207 | G -> |

Variant protein T86235_P26 (SEQ ID NO:476) is encoded by the following transcript(s): T86235_T32 (SEQ ID NO:448), for which the coding portion starts at position 1198 and ends at position 2052. The transcript also has the following SNPs as listed in Table 171 (given according to their position on the nucleotide sequence, with the alternative nucleic acid listed).

TABLE 171
Nucleic acid SNPs

| Polymorphism | SNP position(s) on nucleotide sequence |
|---|---|
| C -> T | 378, 401, 480, 980 |
| C -> G | 766 |
| G -> A | 911, 1212, 2080 |
| A -> G | 1017, 1140, 1489 |
| T -> C | 1398 |
| T -> | 1480 |
| C -> | 1663 |
| G -> | 1817 |

Variant protein T86235_P30 (SEQ ID NO:477) according to the present invention has an amino acid sequence encoded by transcript(s) T86235_T36 (SEQ ID NO:449). One or more alignments to one or more previously published protein sequences are given in the alignment table on the attached CDROM. A brief description of the relationship of the variant protein according to the present invention to each such aligned protein is as follows:

### 1. Comparison Report Between T86235_P30 (SEQ ID NO:477) and Known Protein(s) TAST_HUMAN (SEQ ID NO: 463):

A. An isolated chimeric polypeptide encoding for T86235_P30 (SEQ ID NO:477), comprising a first amino acid sequence being at least about 90% homologous to MTTRQATKDPLLRGVSPTPSKIPVRSQKRTPFPTVTSCAVDQENQDPRRWVQKPPLNIQRPLVDSAGPRPK ARHQAETSQRLVGISQPRNPLEELRPSPRGQNVGPGPPAQT corresponding to amino acids 1-112 of known protein(s) TAST_HUMAN (SEQ ID NO: 463), which also corresponds to amino acids 1-112 of T86235_P30 (SEQ ID NO:477), and a second amino acid sequence being at least about 70%, optionally at least 80%, preferably at least 85%, more preferably at least 90% and most preferably at least 95% homologous to a polypeptide having the sequence GTCWSHGNTASMAE (SEQ ID NO: 534) corresponding to amino acids 113-126 of T86235_P30 (SEQ ID NO:477), wherein said first amino acid sequence and second amino acid sequence are contiguous and in a sequential order.

B. An isolated polypeptide encoding for an edge portion of T86235_P30 (SEQ ID NO:477), comprising an amino acid sequence being at least about 70%, optionally at least about 80%, preferably at least about 85%, more preferably at least about 90% and most preferably at least about 95% homologous to the sequence GTCWSHGNTASMAE (SEQ ID NO: 534) of T86235_P30 (SEQ ID NO:477).

### 2. Comparison Report Between T86235_P30 (SEQ ID NO:477) and Known Protein(s) Q6PJU7_HUMAN (SEQ ID NO: 439):

A. An isolated chimeric polypeptide encoding for T86235_P30 (SEQ ID NO:477), comprising a first amino acid sequence being at least about 90% homologous to MTTRQATKDPLLRGVSPTPSKIPVRSQKRTPFPTVTSCAVDQENQDPRRWVQKPPLNIQRPLVDSAGPRPK ARHQAETSQRLVGISQPRNPLEELRPSPRGQNVGPGPPAQT corresponding to amino acids 1-112 of known protein(s) Q6PJU7_HUMAN (SEQ ID NO: 439), which also corresponds to amino acids 1-112 of T86235_P30 (SEQ ID NO:477), and a second amino acid sequence being at least about 70%, optionally at least 80%, preferably at least 85%, more preferably at least 90% and most preferably at least 95% homologous to a polypeptide having the sequence GTCWSHGNTASMAE (SEQ ID NO: 534) corresponding to amino acids 113-126 of T86235_P30 (SEQ ID NO:477), wherein said first amino acid sequence and second amino acid sequence are contiguous and in a sequential order.

B. An isolated polypeptide encoding for an edge portion of T86235_P30 (SEQ ID NO:477), comprising an amino acid sequence being at least about 70%, optionally at least about 80%, preferably at least about 85%, more preferably at least about 90% and most preferably at least about 95% homologous to the sequence GTCWSHGNTASMAE (SEQ ID NO: 534) of T86235_P30 (SEQ ID NO:477).

### 3. Comparison Report Between T86235_P30 (SEQ ID NO:477) and Known Protein(s) Q8N5B2_HUMAN (SEQ ID NO: 467):

A. An isolated chimeric polypeptide encoding for T86235_P30 (SEQ ID NO:477), comprising a first amino acid sequence being at least about 90% homologous to MTTRQATKDPLLRGVSPTPSKIPVRSQKRTPFPTVTSCAVDQENQDPRRWVQKPPLNIQRPLVDSAGPRPK ARHQAETSQRLVGISQPRNPLEELRPSPRGQNVGPGPPAQT corresponding to amino acids 1-112 of known protein(s) Q8N5B2_HUMAN (SEQ ID NO: 467), which also corresponds to amino acids 1-112 of T86235_P30 (SEQ ID NO:477), and a second amino acid sequence being at least about 70%, optionally at least 80%, preferably at least 85%, more preferably at least 90% and most preferably at least 95% homologous to a polypeptide having the sequence GTCWSHGNTASMAE (SEQ ID NO: 534) corresponding to amino acids 113-126 of T86235_P30 (SEQ ID NO:477), wherein said first amino acid sequence and second amino acid sequence are contiguous and in a sequential order.

B. An isolated polypeptide encoding for an edge portion of T86235_P30 (SEQ ID NO:477), comprising an amino acid sequence being at least about 70%, optionally at least about 80%, preferably at least about 85%, more preferably at least about 90% and most preferably at least about 95% homologous to the sequence GTCWSHGNTASMAE (SEQ ID NO: 534) of T86235_P30 (SEQ ID NO:477).

The localization of the variant protein was determined according to results from a number of different software programs and analyses, including analyses from SignalP and other specialized programs. The variant protein is believed to be located intracellularly with regard to the cell.

Variant protein T86235_P30 (SEQ ID NO:477) also has the following non-silent SNPs (Single Nucleotide Polymorphisms) as listed in Table 172, (given according to their position(s) on the amino acid sequence, with the alternative amino acid(s) listed).

TABLE 172
Amino acid mutations

| SNP position(s) on amino acid | |
|---|---|
| sequence | Alternative amino acid(s) |
| 82 | L -> |

Variant protein T86235_P30 (SEQ ID NO:477) is encoded by the following transcript(s): T86235_T36 (SEQ ID NO:449), for which the coding portion starts at position 298 and ends at position 675. The transcript also has the following SNPs as listed in Table 173 (given according to their position on the nucleotide sequence, with the alternative nucleic acid listed).

TABLE 173
Nucleic acid SNPs

| Polymorphism | SNP position(s) on nucleotide sequence |
|---|---|
| G -> | 240 |
| G -> A | 360 |
| A -> C | 439 |
| T -> | 541 |
| A -> T | 954 |

As noted above, cluster T86235 features 12 segment(s), which were listed in Table 151 above and for which the sequence(s) are given. These segment(s) are portions of nucleic acid sequence(s) which are described herein separately because they are of particular interest. A description of some of these segments according to the present invention is now provided.

Segment cluster T86235_N2 (SEQ ID NO:450) according to the present invention is supported by 73 libraries. The number of libraries was determined as previously described. This segment can be found in the following transcript(s): T86235_T11 (SEQ ID NO:443), T86235_T13 (SEQ ID NO:444), T86235_T15 (SEQ ID NO:445), T86235_T18 (SEQ ID NO:446), T86235_T36 (SEQ ID NO:449), T86235_T5 (SEQ ID NO:440), T86235_T6 (SEQ ID NO:441) and T86235_T7 (SEQ ID NO:442). Table 174 below describes the starting and ending position of this segment on each transcript.

TABLE 174
Segment location on transcripts

| | Segment | Segment |
|---|---|---|
| Transcript name | starting position | ending position |
| T86235_T11 (SEQ ID NO: 443) | 79 | 292 |
| T86235_T13 (SEQ ID NO: 444) | 79 | 292 |
| T86235_T15 (SEQ ID NO: 445) | 79 | 292 |
| T86235_T18 (SEQ ID NO: 446) | 79 | 292 |
| T86235_T36 (SEQ ID NO: 449) | 79 | 292 |
| T86235_T5 (SEQ ID NO: 440) | 79 | 292 |
| T86235_T6 (SEQ ID NO: 441) | 79 | 292 |
| T86235_T7 (SEQ ID NO: 442) | 79 | 292 |

Segment cluster T86235_N19 (SEQ ID NO:451) according to the present invention is supported by 3 libraries. The number of libraries was determined as previously described. This segment can be found in the following transcript(s): T86235_T26 (SEQ ID NO:447). Table 175 below describes the starting and ending position of this segment on each transcript.

TABLE 175
Segment location on transcripts

| | Segment | Segment |
|---|---|---|
| Transcript name | starting position | ending position |
| T86235_T26 (SEQ ID NO: 447) | 1 | 134 |

Microarray (chip) data is also available for this segment as follows. As described above with regard to the cluster itself, various oligonucleotides were tested for being differentially expressed in various disease conditions, particularly cancer. The following oligonucleotides were found to hit this segment, shown in Table 176.

TABLE 176
Oligonucleotides related to this segment

| Oligonucleotide name | Overexpressed in cancers | Chip reference |
|---|---|---|
| T86235_0_74_63 | lung malignant tumors | LUN |
| (SEQ ID NO: 492) | | |

Segment cluster T86235_N33 (SEQ ID NO:452) according to the present invention is supported by 2 libraries. The number of libraries was determined as previously described. This segment can be found in the following transcript(s): T86235_T32 (SEQ ID NO:448). Table 177 below describes the starting and ending position of this segment on each transcript.

TABLE 177
Segment location on transcripts

| | Segment | Segment |
|---|---|---|
| Transcript name | starting position | ending position |
| T86235_T32 (SEQ ID NO: 448) | 1 | 294 |

Segment cluster T86235_N39 (SEQ ID NO:453) according to the present invention is supported by 9 libraries. The number of libraries was determined as previously described. This segment can be found in the following transcript(s): T86235_T13 (SEQ ID NO:444), T86235_T15 (SEQ ID NO:445) and T86235_T32 (SEQ ID NO:448). Table 178 below describes the starting and ending position of this segment on each transcript.

TABLE 178
Segment location on transcripts

| | Segment | Segment |
|---|---|---|
| Transcript name | starting position | ending position |
| T86235_T13 (SEQ ID NO: 444) | 1462 | 1863 |
| T86235_T15 (SEQ ID NO: 445) | 1462 | 1863 |
| T86235_T32 (SEQ ID NO: 448) | 568 | 969 |

Segment cluster T86235_N41 (SEQ ID NO:454) according to the present invention is supported by 4 libraries. The number of libraries was determined as previously described. This segment can be found in the following transcript(s): T86235_T13 (SEQ ID NO:444) and T86235_T5 (SEQ ID NO:440). Table 179 below describes the starting and ending position of this segment on each transcript.

TABLE 179
Segment location on transcripts

| | Segment | Segment |
|---|---|---|
| Transcript name | starting position | ending position |
| T86235_T13 (SEQ ID NO: 444) | 1999 | 2151 |
| T86235_T5 (SEQ ID NO: 440) | 1597 | 1749 |

Segment cluster T86235_N53 (SEQ ID NO:455) according to the present invention is supported by 22 libraries. The number of libraries was determined as previously described. This segment can be found in the following transcript(s): T86235_T15 (SEQ ID NO:445), T86235_T18 (SEQ ID NO:446), T86235_T6 (SEQ ID NO:441) and T86235_T7 (SEQ ID NO:442). Table 180 below describes the starting and ending position of this segment on each transcript.

TABLE 180
Segment location on transcripts

| | Segment | Segment |
|---|---|---|
| Transcript name | starting position | ending position |
| T86235_T15 (SEQ ID NO: 445) | 2798 | 3067 |
| T86235_T18 (SEQ ID NO: 446) | 2309 | 2578 |
| T86235_T6 (SEQ ID NO: 441) | 2309 | 2578 |
| T86235_T7 (SEQ ID NO: 442) | 2396 | 2665 |

Segment cluster T86235_N55 (SEQ ID NO:456) (T86235_N55 (SEQ ID NO:456) segment name in experimental results is T86235_seg 44 (SEQ ID NO: 285)) according to the present invention is supported by 5 libraries. The number of libraries was determined as previously described. This segment can be found in the following transcript(s): T86235_T11 (SEQ ID NO:443) and T86235_T18 (SEQ ID NO:446). Table 181 below describes the starting and ending position of this segment on each transcript.

TABLE 181
Segment location on transcripts

| | Segment | Segment |
|---|---|---|
| Transcript name | starting position | ending position |
| T86235_T11 (SEQ ID NO: 443) | 2503 | 2667 |
| T86235_T18 (SEQ ID NO: 446) | 2773 | 2937 |

According to an optional embodiment of the present invention, short segments related to the above cluster are also provided. These segments are up to about 120 bp in length, and so are included in a separate description.

Segment cluster T86235 N12 (SEQ ID NO:457) according to the present invention is supported by 9 libraries. The number of libraries was determined as previously described. This segment can be found in the following transcript(s): T86235_T36 (SEQ ID NO:449). Table 182 below describes the starting and ending position of this segment on each transcript.

TABLE 182
Segment location on transcripts

| | Segment | Segment |
|---|---|---|
| Transcript name | starting position | ending position |
| T86235_T36 (SEQ ID NO: 449) | 635 | 739 |

Segment cluster T86235_N13 (SEQ ID NO:458) according to the present invention is supported by 10 libraries. The number of libraries was determined as previously described. This segment can be found in the following transcript(s): T86235_T36 (SEQ ID NO:449). Table 183 below describes the starting and ending position of this segment on each transcript.

TABLE 183
Segment location on transcripts

| | Segment | Segment |
|---|---|---|
| Transcript name | starting position | ending position |
| T86235_T36 (SEQ ID NO: 449) | 740 | 751 |

Segment cluster T86235_N14 (SEQ ID NO:459) according to the present invention is supported by 18 libraries. The number of libraries was determined as previously described. This segment can be found in the following transcript(s): T86235_T36 (SEQ ID NO:449). Table 184 below describes the starting and ending position of this segment on each transcript.

TABLE 184
Segment location on transcripts

| | Segment | Segment |
|---|---|---|
| Transcript name | starting position | ending position |
| T86235_T36 (SEQ ID NO: 449) | 752 | 808 |

Segment cluster T86235_N46 (SEQ ID NO:460) according to the present invention is supported by 30 libraries. The number of libraries was determined as previously described. This segment can be found in the following transcript(s): T86235_T15 (SEQ ID NO:445) and T86235_T7 (SEQ ID NO:442). Table 185 below describes the starting and ending position of this segment on each transcript.

TABLE 185
Segment location on transcripts

| | Segment | Segment |
|---|---|---|
| Transcript name | starting position | ending position |
| T86235_T15 (SEQ ID NO: 445) | 2428 | 2514 |
| T86235_T7 (SEQ ID NO: 442) | 2026 | 2112 |

Segment cluster T86235_N47 (SEQ ID NO:461) according to the present invention is supported by 33 libraries. The number of libraries was determined as previously described. This segment can be found in the following transcript(s): T86235_T11 (SEQ ID NO:443), T86235_T13 (SEQ ID NO:444), T86235_T15 (SEQ ID NO:445), T86235_T18 (SEQ ID NO:446), T86235_T26 (SEQ ID NO:447), T86235_T32 (SEQ ID NO:448), T86235_T5 (SEQ ID NO:440), T86235_T6 (SEQ ID NO:441) and T86235_T7 (SEQ ID NO:442). Table 186 below describes the starting and ending position of this segment on each transcript.

TABLE 186
Segment location on transcripts

| | Segment | Segment |
|---|---|---|
| Transcript name | starting position | ending position |
| T86235_T11 (SEQ ID NO: 443) | 2026 | 2037 |
| T86235_T13 (SEQ ID NO: 444) | 2581 | 2592 |
| T86235_T15 (SEQ ID NO: 445) | 2515 | 2526 |
| T86235_T18 (SEQ ID NO: 446) | 2026 | 2037 |
| T86235_T26 (SEQ ID NO: 447) | 1526 | 1537 |
| T86235_T32 (SEQ ID NO: 448) | 1534 | 1545 |
| T86235_T5 (SEQ ID NO: 440) | 2179 | 2190 |
| T86235_T6 (SEQ ID NO: 441) | 2026 | 2037 |
| T86235_T7 (SEQ ID NO: 442) | 2113 | 2124 |

Expression of Homo sapiens trophinin associated protein (tastin) (TROAP) transcripts detectable by or according to segments as described below was measured by real time PCR: seg44—T86235_seg44 (SEQ ID NO: 285) amplicon and primers T86235_seg44F (SEQ ID NO: 283) and T86235_seg44R (SEQ ID NO: 284); seg46-47WT—T86235_seg46-47WT (SEQ ID NO: 288) amplicon and primers T86235_seg46-47WTF (SEQ ID NO: 286) and T86235_seg46-47WTR (SEQ ID NO: 287).

The sequences of the corresponding amplicons and primers are given below:

Forward Primer T86235_seg44F (SEQ ID NO: 283):
AGACTCCAACCCACAGCCC
Reverse Primer T86235_seg44R (SEQ ID NO: 284):
CAGCTCAGCCAACCTTGCA
Amplicon T86235_seg44 (SEQ ID NO: 285):
AGACTCCAACCCACAGCCCAGCTGTGGCTGCACAGTGAGCCTGATGGGAG
GTGGGGAACAGGGACAGGGGGCCACCTGGGCTTCTTCACAGAGAGGTCAG
CAGGAAGGCTTGGCTACAGTGCAAGGTTGGCTGAGCTG
Forward Primer T86235_seg46-47WTF
(SEQ ID NO: 286):
GTACGTGAGCCCTGCCCT
Reverse Primer T86235_seg46-47WTR
(SEQ ID NO: 287):
CTCAAGCTGTTCCTGGCGA
Amplicon T86235_seg46-47WT (SEQ ID NO: 288):
GTACCTGAGCCCTGCCCTCCAGCAGAACCCAGGCCCCTAGAGTCCTACTG
TAGGATTGAGCCTGAGATACCGGAGTCCTCTCGCCAGGAACAGCTTGAG

The below table 187 gives a conversion between the terms for segments (nodes) in the experimental results and those listed earlier in the description for cluster T86235:

TABLE 187

| Segment name for | Segment name within the description |
|---|---|
| T86235 in experimental results | for cluster T86235 report |
| seg 44 (SEQ ID NO: 285) | seg 55 |
| 46-47WT (SEQ ID NO: 288) | 46-47WT |

Expression of Homo sapiens Trophinin Associated Protein (tastin) (TROAP) T86235 Transcripts which are Detectable by Amplicon as Depicted in Sequence Name T86235_seg44 (SEQ ID NO: 285) in Normal and Cancerous Breast Tissues

Expression of Homo sapiens trophinin associated protein (tastin) (TROAP) transcripts detectable by or according to seg44—T86235_seg44 (SEQ ID NO: 285) amplicon and primers T86235_seg44F (SEQ ID NO: 283) and T86235_seg44R (SEQ ID NO: 284) was measured by real time PCR. In parallel the expression of four housekeeping genes—G6PD (GenBank Accession No. NM_{—}000402 (SEQ ID NO:339); G6PD amplicon (SEQ ID NO: 311)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 57, 59, 60, 63, 66, 64, 56, 65, 67 and 58, Table 1_{—}3 above), to obtain a value of fold up-regulation for each sample relative to the median of the normal PM samples.

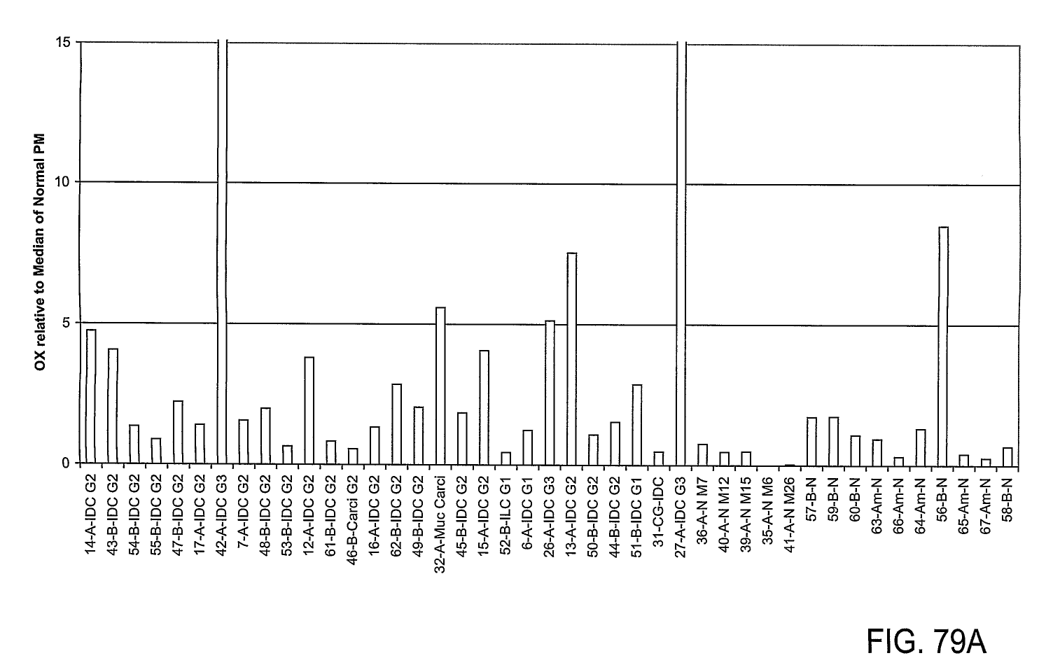

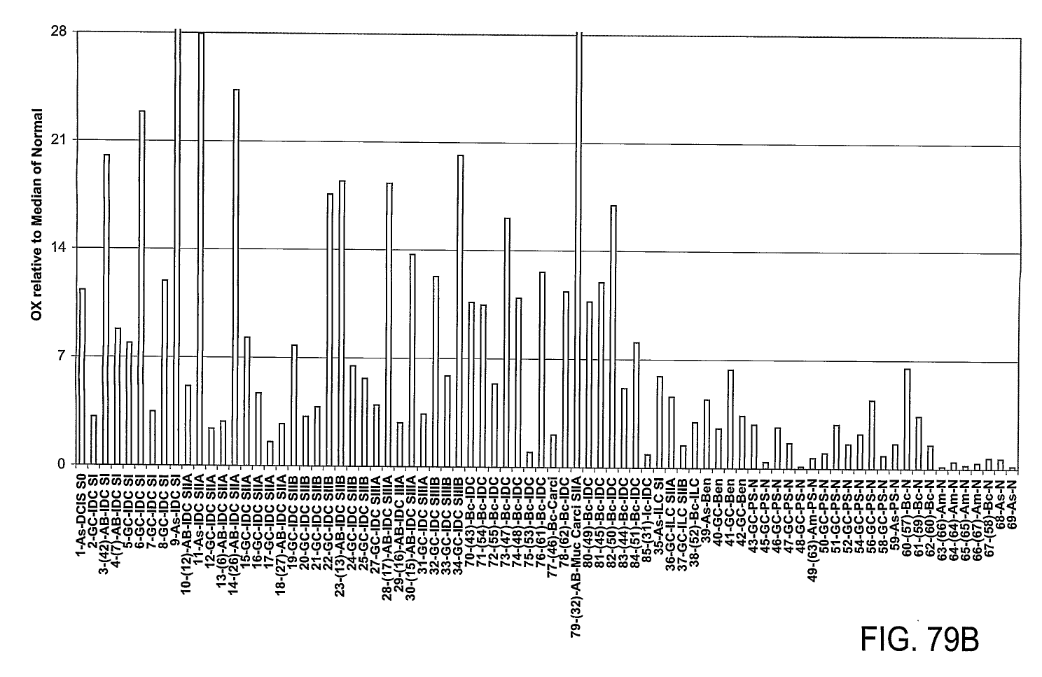

FIG. 79A is a histogram showing over expression of the Homo sapiens trophinin associated protein (tastin) (TROAP) transcripts detectable by or according to T86235_seg44 (SEQ ID NO: 285) in cancerous Breast samples relative to the normal samples.

As is evident from FIG. 79A, the expression of Homo sapiens trophinin associated protein (tastin) (TROAP) transcripts detectable by the above amplicon in cancer samples was higher than in the non-cancerous samples (sample numbers 57, 59, 60, 63, 66, 64, 56, 65, 67 and 58, Table 1_{—}3 above). Notably an over-expression of at least 5 fold was found in 5 out of 28 adenocarcinoma samples.

Primer pairs are also optionally and preferably encompassed within the present invention; for example, for the above experiment, the following primer pair was used as a non-limiting illustrative example only of a suitable primer pair: T86235_seg44F (SEQ ID NO: 283) forward primer; and T86235_seg44R (SEQ ID NO: 284) reverse primer.

The present invention also preferably encompasses any amplicon obtained through the use of any suitable primer pair; for example, for the above experiment, the following amplicon was obtained as a non-limiting illustrative example only of a suitable amplicon: T86235_seg44 (SEQ ID NO: 285).

Expression of Homo sapiens trophinin associated protein (tastin) (TROAP) transcripts detectable by or according to seg44—T86235_seg44 (SEQ ID NO: 285) amplicon and primers T86235_seg44F (SEQ ID NO: 283) and T86235_seg44R (SEQ ID NO: 284) was further measured by real time PCR using enlarged panel as shown in Table 1_{—}7. In parallel the expression of four housekeeping genes—G6PD (GenBank Accession No. NM_{—}000402 (SEQ ID NO:339); G6PD amplicon (SEQ ID NO: 311)), RPL19 (GenBank Accession No. NM_{—}000981 (SEQ ID NO:342); RPL19 amplicon (SEQ ID NO: 320)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 43, 45, 46, 47, 48, 49, 50, 51, 52, 54, 56, 58, 59, 60, 61, 62, 63, 64, 65, 66, 67, 68 and 69, Table 1_{—}7 above), to obtain a value of fold up-regulation for each sample relative to the median of the normal samples.

FIG. 79B is a histogram showing over expression of the Homo sapiens trophinin associated protein (tastin) (TROAP) transcripts in cancerous Breast samples relative to the normal samples.

As is evident from FIG. 79B, the expression of Homo sapiens trophinin associated protein (tastin) (TROAP) transcripts detectable by the above amplicon in cancer samples was significantly higher than in the non-cancerous samples (sample numbers 39-42, 43, 45, 46, 47, 48, 49, 50, 51, 52, 54, 56, 58, 59, 60, 61, 62, 63, 64, 65, 66, 67, 68 and 69, Table 1_{—}7 above). Notably an over-expression of at least 7 fold was found in 28 out of 53 adenocarcinoma samples.

Statistical analysis was applied to verify the significance of these results, as described below.

The P value for the difference in the expression levels of Homo sapiens trophinin associated protein (tastin) (TROAP) transcripts detectable by the above amplicon in Breast cancer samples versus the normal tissue samples was determined by T test as 1.50e-03.

Threshold of 7 fold over expression was found to differentiate between cancer and normal samples with P value of 3.11e-07 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T86235_seg44F (SEQ ID NO: 283) forward primer; and T86235_seg44R (SEQ ID NO: 284) reverse primer.

The present invention also preferably encompasses any amplicon obtained through the use of any suitable primer pair; for example, for the above experiment, the following amplicon was obtained as a non-limiting illustrative example only of a suitable amplicon: T86235_seg44 (SEQ ID NO: 285).

Expression of Homo sapiens Trophinin Associated Protein (Tastin) (TROAP) T86235 Transcripts which are Detectable by Amplicon as Depicted in Sequence Name T86235_seg44 (SEQ ID NO: 285) in Normal and Cancerous Ovary Tissues

Expression of Homo sapiens trophinin associated protein (tastin) (TROAP) transcripts detectable by or according to seg44—T86235_seg44 (SEQ ID NO: 285) amplicon and primers T86235_seg44F (SEQ ID NO: 283) and T86235_seg44R (SEQ ID NO: 284) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and GAPDH (GenBank Accession No. BC026907 (SEQ ID NO:338); GAPDH amplicon (SEQ ID NO: 308)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 45, 71 and 48, Table 1_{—}1 above), to obtain a value of fold up-regulation for each sample relative to the median of the normal PM samples.

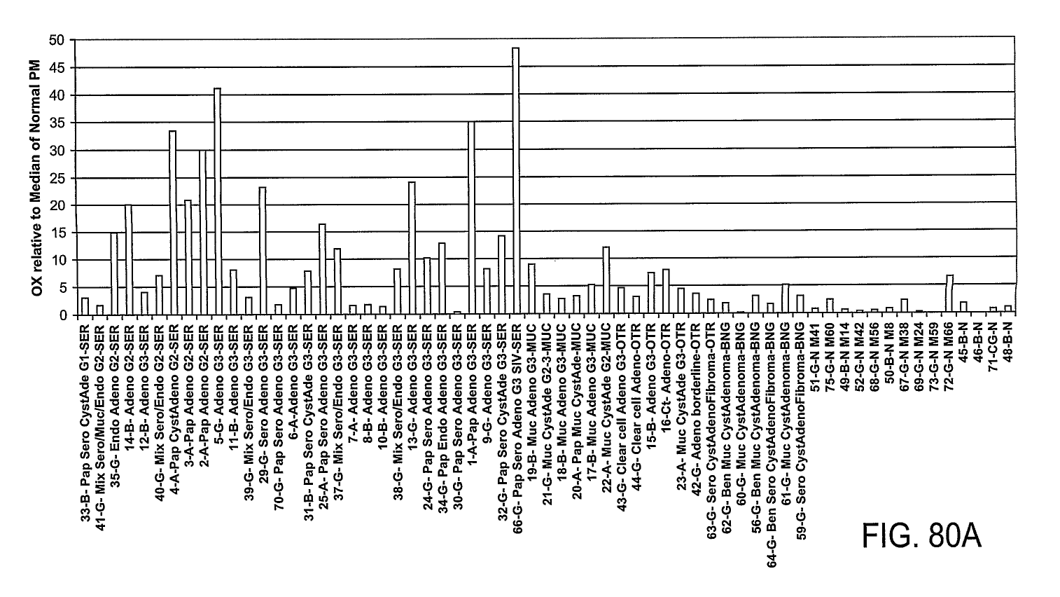

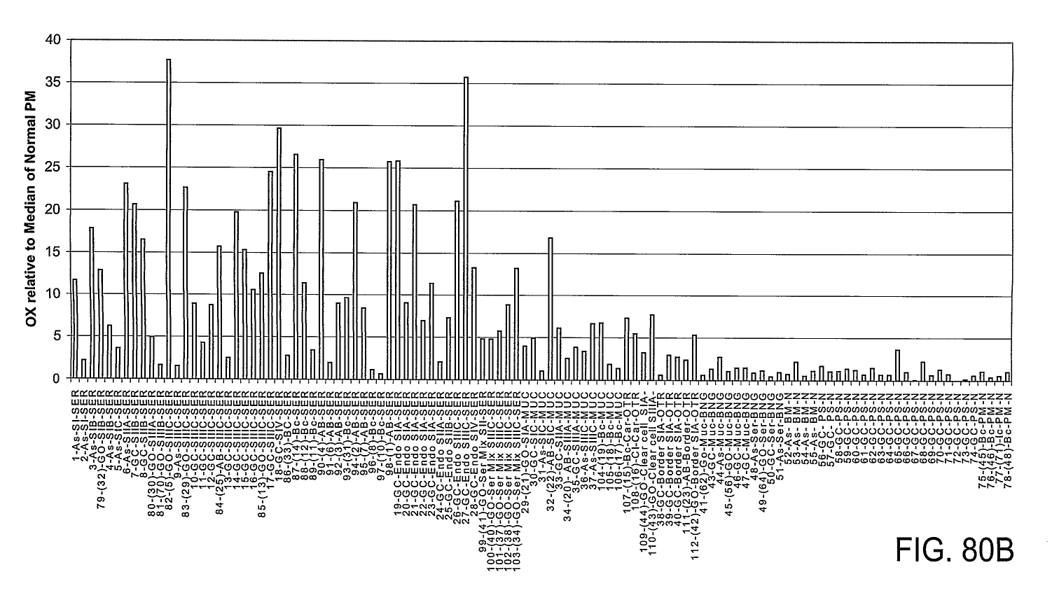

FIG. 80A is a histogram showing over expression of the Homo sapiens trophinin associated protein (tastin) (TROAP) transcripts detectable by or according to T86235_seg44 (SEQ ID NO: 285) in cancerous Ovary samples relative to the normal samples.

As is evident from FIG. 80A, the expression of Homo sapiens trophinin associated protein (tastin) (TROAP) transcripts detectable by the above amplicon in adenocarcinoma samples was higher than in the non-cancerous samples (sample numbers 45, 71 and 48, Table 1_{—}1 above). Notably an over-expression of at least 5 fold was found in 25 out of 43 adenocarcinoma samples, specifically in 20 out of 30 serous carcinoma samples and in 3 out of 6 mucinous carcinoma samples.

Statistical analysis was applied to verify the significance of these results, as described below.

The P value for the difference in the expression levels of Homo sapiens trophinin associated protein (tastin) (TROAP) transcripts detectable by the above amplicon in Ovary serous carcinoma samples versus the normal tissue samples was determined by T test as 8.05e-006. The P value for the difference in the expression levels of Homo sapiens trophinin associated protein (tastin) (TROAP) transcripts detectable by the above amplicon in Ovary mucinous carcinoma samples versus the normal tissue samples was determined by T test as 2.55e-002. The P value for the difference in the expression levels of Homo sapiens trophinin associated protein (tastin) (TROAP) transcripts detectable by the above amplicon in all ovary adenocarcinoma samples versus the normal tissue samples was determined by T test as 1.00e-006.

The above values demonstrate statistical significance of the results.

Primer pairs are also optionally and preferably encompassed within the present invention; for example, for the above experiment, the following primer pair was used as a non-limiting illustrative example only of a suitable primer pair: T86235_seg44F (SEQ ID NO: 283) forward primer; and T86235_seg44R (SEQ ID NO: 284) reverse primer.

The present invention also preferably encompasses any amplicon obtained through the use of any suitable primer pair; for example, for the above experiment, the following amplicon was obtained as a non-limiting illustrative example only of a suitable amplicon: T86235_seg44 (SEQ ID NO: 285).

Expression of sapiens trophinin associated protein (tastin) (TROAP) transcripts detectable by or according to seg44—T86235_seg44 (SEQ ID NO: 285) amplicon and primers T86235_seg44F (SEQ ID NO: 283) and T86235_seg44R (SEQ ID NO: 284) was further measured by real time PCR using enlarged panel as shown in Table 1_{—}5. In parallel the expression of three housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)), G6PD (GenBank Accession No. NM_{—}000402 (SEQ ID NO:339); G6PD amplicon (SEQ ID NO: 311)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above), to obtain a value of fold up-regulation for each sample relative to the median of the normal samples.

FIG. 80B is a histogram showing over expression of the sapiens trophinin associated protein (tastin) (TROAP) transcripts in cancerous Ovary samples relative to the normal samples.

As is evident from FIG. 80B, the expression of sapiens trophinin associated protein (tastin) (TROAP) transcripts detectable by the above amplicon in adenocarcinoma samples was significantly higher than in the non-cancerous samples (sample numbers 41, 43-78, Table 1_{—}5 above). Notably an over-expression of at least 5 fold was found in 46 out of 74 adenocarcinoma samples, specifically in 26 out of 38 serous carcinoma samples, in 9 out of 10 endometroid type adenocarcinoma, in 4 out of 12 mucinous carcinoma samples.

Statistical analysis was applied to verify the significance of these results, as described below.

The P value for the difference in the expression levels of sapiens trophinin associated protein (tastin) (TROAP) transcripts detectable by the above amplicon in Ovary adenocarcinoma samples versus the normal tissue samples was determined by T test as 1.21e-13. The P value for the difference in the expression levels of sapiens trophinin associated protein (tastin) (TROAP) transcripts detectable by the above amplicon in Ovary serous carcinoma samples versus the normal tissue samples was determined by T test as 3.60e-09. The P value for the difference in the expression levels of sapiens trophinin associated protein (tastin) (TROAP) transcripts detectable by the above amplicon in Ovary mucinous carcinoma samples versus the normal tissue samples was determined by T test as 9.40e-03.

Threshold of 5 fold over expression was found to differentiate between adenocarcinoma and normal samples with P value of 5.08e-12 as checked by exact Fisher test. Threshold of 5 fold over expression was found to differentiate between serous carcinoma and normal samples with P value of 2.68e-11 as checked by exact Fisher test. Threshold of 5 fold over expression was found to differentiate between mucinous carcinoma and normal samples with P value of 2.34e-03 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T86235_seg44F (SEQ ID NO: 283) forward primer; and T86235_seg44R (SEQ ID NO: 284) reverse primer.

The present invention also preferably encompasses any amplicon obtained through the use of any suitable primer pair; for example, for the above experiment, the following amplicon was obtained as a non-limiting illustrative example only of a suitable amplicon: T86235_seg44 (SEQ ID NO: 285).

Expression of Homo sapiens Trophinin Associated Protein (tastin) (TROAP) T86235 Transcripts which are Detectable by Amplicon as Depicted in Sequence Name T86235_seg44 (SEQ ID NO: 285) in Different Normal Tissues

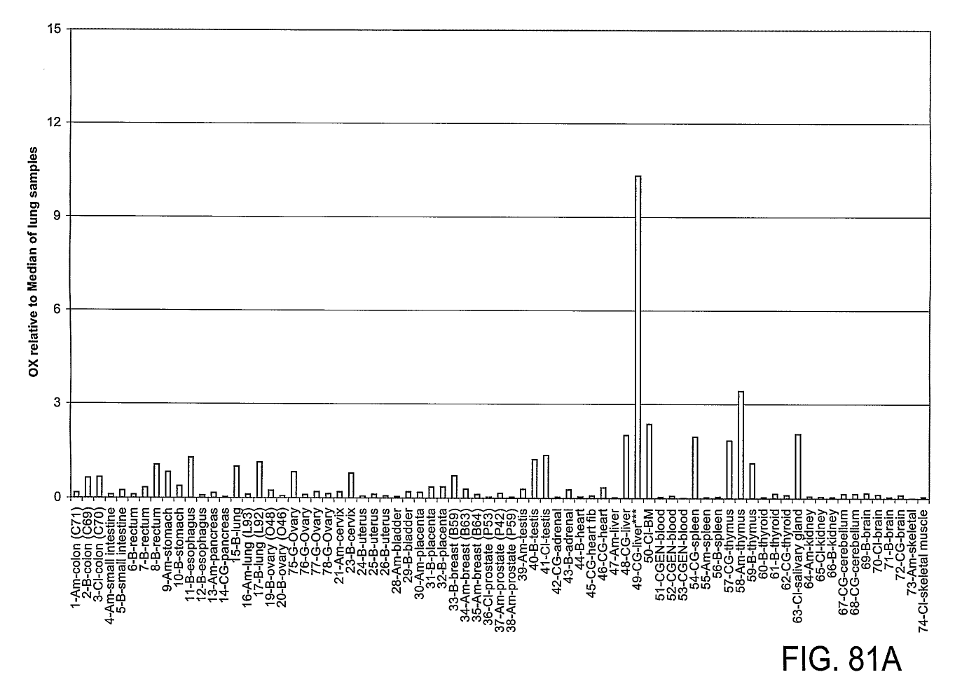

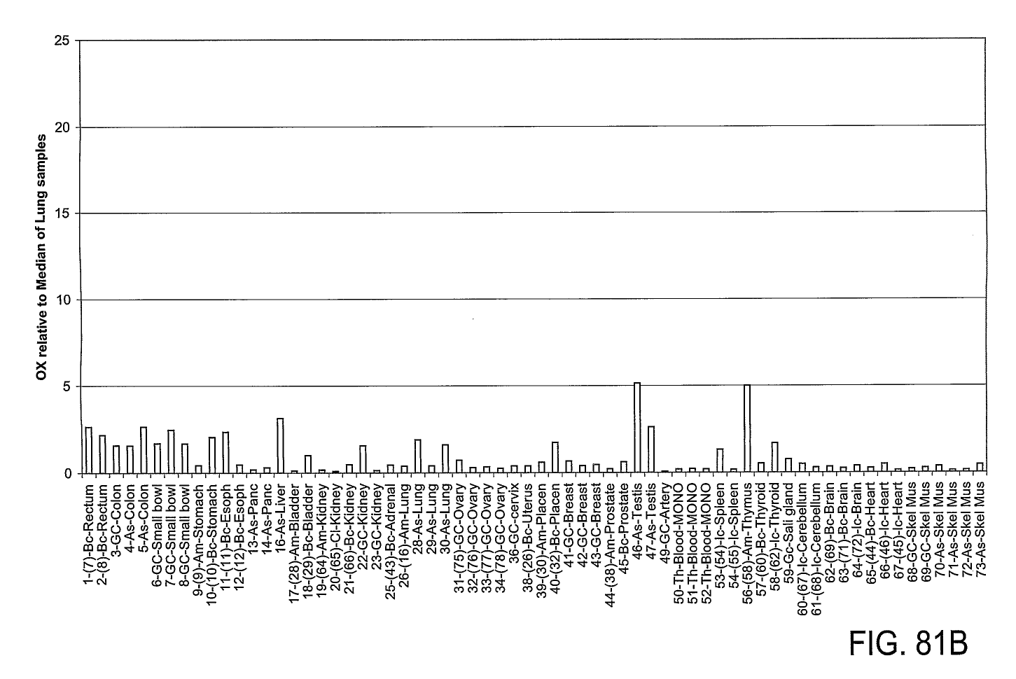

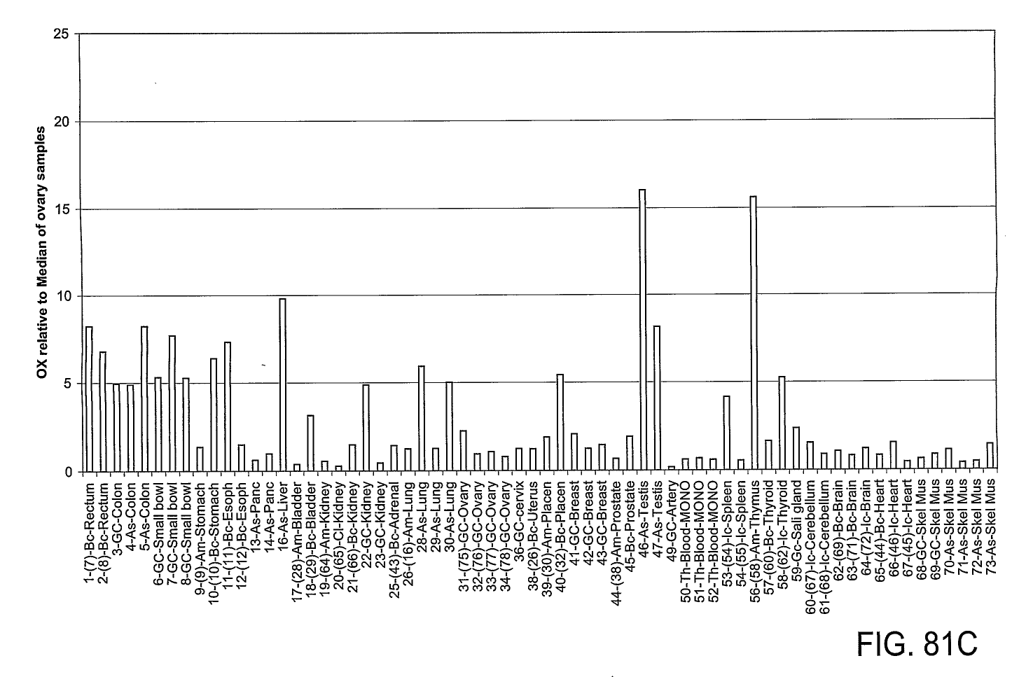

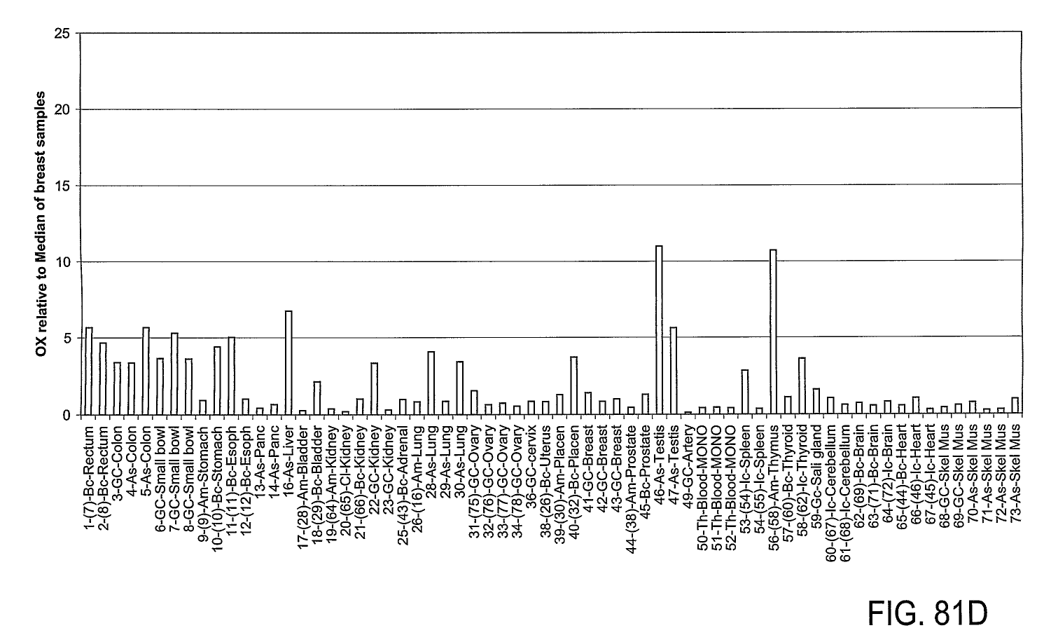

Expression of Homo sapiens trophinin associated protein (tastin) (TROAP) transcripts detectable by or according to seg44—T86235_seg44 (SEQ ID NO: 285) amplicon and primers T86235_seg44F (SEQ ID NO: 283) and T86235_seg44R (SEQ ID NO: 284) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the geometric mean of the quantities of the housekeeping genes. The normalized quantity of each RT sample was then divided by the median of the quantities of the lung samples (sample numbers 15, 16 and 17, Table 1_{—}9 above), to obtain a value of relative expression of each sample relative to the median of the lung samples. FIG. 81A shows expression of Homo sapiens trophinin associated protein (tastin) (TROAP) T86235 transcripts which are detectable by amplicon as depicted in sequence name T86235_seg44 (SEQ ID NO: 285) in different normal tissues.

Expression of Homo sapiens trophinin associated protein (tastin) (TROAP) transcripts detectable by or according to seg44—T86235_seg44 (SEQ ID NO: 285) amplicon and primers T86235_seg44F (SEQ ID NO: 283) and T86235_seg44R (SEQ ID NO: 284) was further measured by real time PCR using updated panel as shown in Table 1_{—}10. In parallel the expression of three housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), and TATA box (GenBank Accession No. NM_{—}003194 (SEQ ID NO:343); TATA amplicon (SEQ ID NO: 323)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of:
A. The Lung samples (sample numbers 26, 28, 29 and 30, Table 1_{—}10 above), to obtain a value of relative expression of each sample relative to the median of the Lung samples—as presented in FIG. 81B
B. The ovary samples (sample numbers 31-34, Table 1_{—}10 above), to obtain a value of relative expression of each sample relative to the median of the Ovary samples—as presented in FIG. 81C.
C. The breast samples (sample numbers 41-43, Table 1_{—}10 above), to obtain a value of relative expression of each sample relative to the median of the Breast samples—as presented in FIG. 81D.
   Expression of Homo sapiens Trophinin Associated Protein (tastin) (TROAP) T86235 Transcripts which are Detectable by Amplicon as Depicted in Sequence Name T86235_seg46-47WT (SEQ ID NO: 288) in Normal and Cancerous Breast Tissues

Expression of Homo sapiens trophinin associated protein (tastin) (TROAP) transcripts detectable by or according to seg46-47WT-T86235_seg46-47WT (SEQ ID NO: 288) amplicon and primers T86235_seg46-47WTF (SEQ ID NO: 286) and T86235_seg46-47WTR (SEQ ID NO: 287) was measured by real time PCR. In parallel the expression of four housekeeping genes—G6PD (GenBank Accession No. NM_{—}000402 (SEQ ID NO:339); G6PD amplicon (SEQ ID NO: 311)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 57, 59, 60, 63, 66, 64, 56, 65, 67 and 58, Table 1_{—}3 above), to obtain a value of fold up-regulation for each sample relative to the median of the normal PM samples.

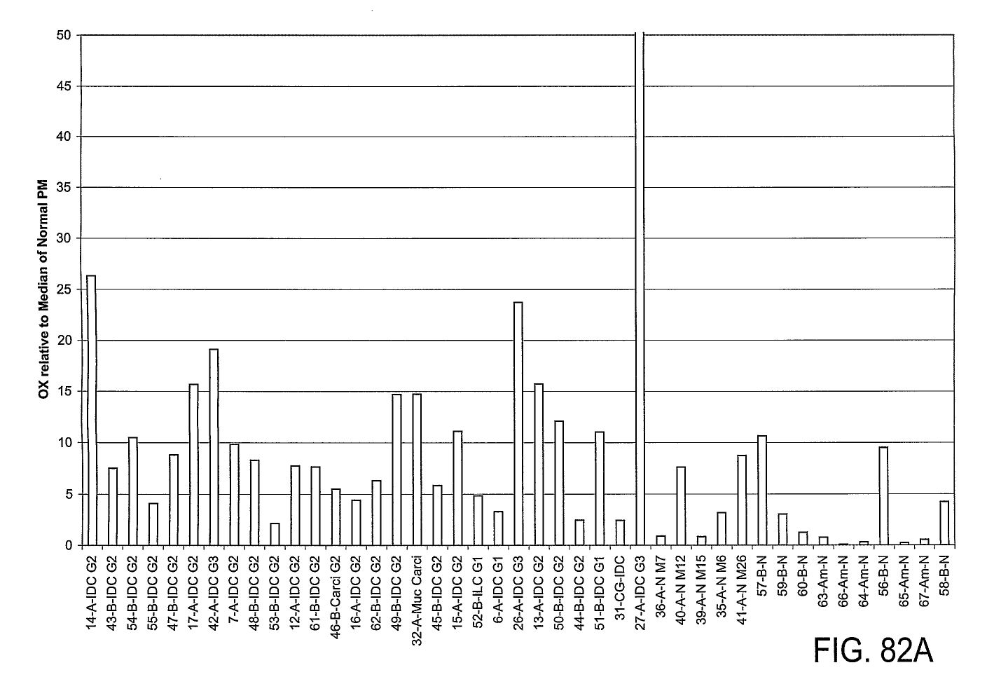

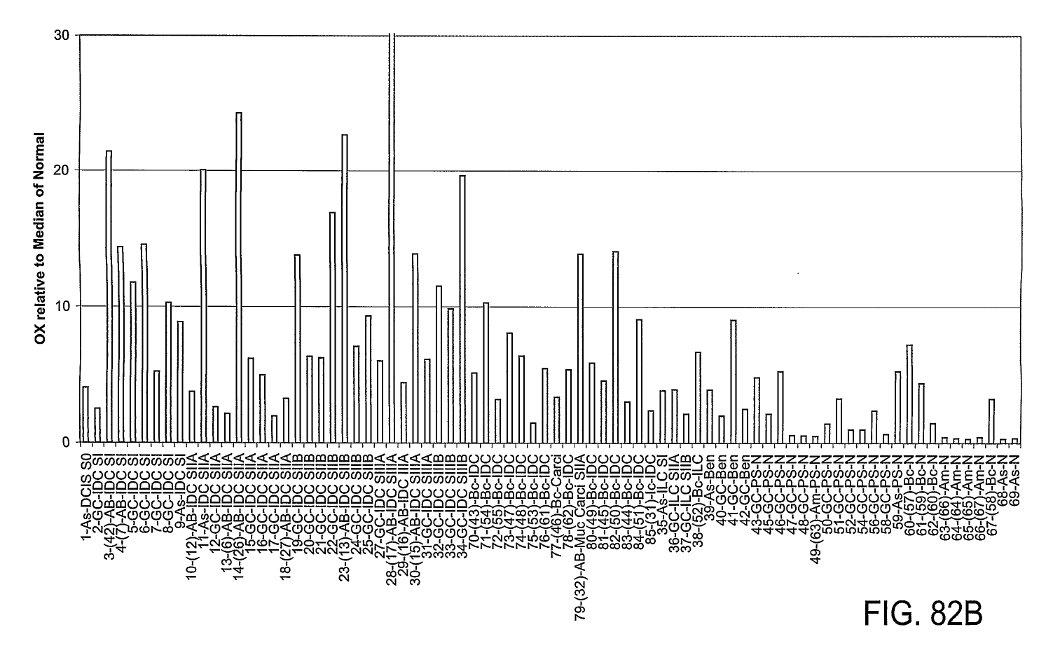

FIG. 82A is a histogram showing over expression of the Homo sapiens trophinin associated protein (tastin) (TROAP) transcripts detectable by or according to T86235_seg46-47WT (SEQ ID NO: 288) in cancerous Breast samples relative to the normal samples.

As is evident from FIG. 82A, the expression of Homo sapiens trophinin associated protein (tastin) (TROAP) transcripts detectable by the above amplicon in cancer samples was significantly higher than in the non-cancerous samples (sample numbers 57, 59, 60, 63, 66, 64, 56, 65, 67 and 58, Table 1_{—}3 above). Notably an over-expression of at least 5 fold was found in 21 out of 28 adenocarcinoma samples.

Statistical analysis was applied to verify the significance of these results, as described below.

The P value for the difference in the expression levels of Homo sapiens trophinin associated protein (tastin) (TROAP) transcripts detectable by the above amplicon in Breast cancer samples versus the normal tissue samples was determined by T test as 3.22e-02.

Threshold of 5 fold over expression was found to differentiate between cancer and normal samples with P value of 3.66e-03 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T86235_seg46-47WTF (SEQ ID NO: 286) forward primer; and T86235_seg46-47WTR (SEQ ID NO: 287) reverse primer.

The present invention also preferably encompasses any amplicon obtained through the use of any suitable primer pair; for example, for the above experiment, the following amplicon was obtained as a non-limiting illustrative example only of a suitable amplicon: T86235_seg46-47WT (SEQ ID NO: 288).

Expression of Homo sapiens trophinin associated protein (tastin) (TROAP) transcripts detectable by or according to seg46-47WT—T86235_seg46-47WT (SEQ ID NO: 288) amplicon and primers T86235_seg46-47WTF (SEQ ID NO: 286) and T86235_seg46-47WTR (SEQ ID NO: 287) was further measured by real time PCR using enlarged panel as shown in Table 1_{—}7. In parallel the expression of four housekeeping genes—G6PD (GenBank Accession No. NM_{—}000402 (SEQ ID NO:339); G6PD amplicon (SEQ ID NO: 311)), RPL19 (GenBank Accession No. NM_{—}000981 (SEQ ID NO:342); RPL19 amplicon (SEQ ID NO: 320)), PBGD (GenBank Accession No. BC019323 (SEQ ID NO:336); amplicon—PBGD-amplicon (SEQ ID NO: 302)) and SDHA (GenBank Accession No. NM_{—}004168 (SEQ ID NO:335); amplicon—SDHA-amplicon (SEQ ID NO:299)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 43, 45, 46, 47, 48, 49, 50, 51, 52, 54, 56, 58, 59, 60, 61, 62, 63, 64, 65, 66, 67, 68 and 69, Table 1_{—}7 above), to obtain a value of fold up-regulation for each sample relative to the median of the normal samples.

FIG. 82B is a histogram showing over expression of the Homo sapiens trophinin associated protein (tastin) (TROAP) transcripts in cancerous Breast samples relative to the normal samples.

As is evident from FIG. 82B, the expression of Homo sapiens trophinin associated protein (tastin) (TROAP) transcripts detectable by the above amplicon in cancer samples was significantly higher than in the non-cancerous samples (sample numbers 39-42, 43, 45, 46, 47, 48, 49, 50, 51, 52, 54, 56, 58, 59, 60, 61, 62, 63, 64, 65, 66, 67, 68 and 69, Table 117 above). Notably an over-expression of at least 10 fold was found in 17 out of 53 adenocarcinoma samples.

Statistical analysis was applied to verify the significance of these results, as described below.

The P value for the difference in the expression levels of Homo sapiens trophinin associated protein (tastin) (TROAP) transcripts detectable by the above amplicon in Breast cancer samples versus the normal tissue samples was determined by T test as 1.20e-08.

Threshold of 5 fold over expression was found to differentiate between cancer and normal samples with P value of 3.18e-04 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T86235_seg46-47WTF (SEQ ID NO: 286) forward primer; and T86235_seg46-47WTR (SEQ ID NO: 287) reverse primer.

The present invention also preferably encompasses any amplicon obtained through the use of any suitable primer pair; for example, for the above experiment, the following amplicon was obtained as a non-limiting illustrative example only of a suitable amplicon: T86235_seg46-47WT (SEQ ID NO: 288).

Expression of Homo sapiens Trophinin Associated Protein (tastin) (TROAP) T86235 Transcripts which are Detectable by Amplicon as Depicted in Sequence Name T86235_seg46-47WT (SEQ ID NO: 288) in Normal and Cancerous Lung Tissues

Expression of Homo sapiens trophinin associated protein (tastin) (TROAP) transcripts detectable by or according to seg46-47WT—T86235_seg46-47WT (SEQ ID NO: 288) amplicon and primers T86235_seg46-47WTF (SEQ ID NO: 286) and T86235_seg46-47WTR (SEQ ID NO: 287) was measured by real time PCR. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 47, 48, 49, 50, 90, 91, 92, 93, 96, 97, 98 and 99, Table 1_{—}2 above), to obtain a value of fold up-regulation for each sample relative to the median of the normal PM samples.

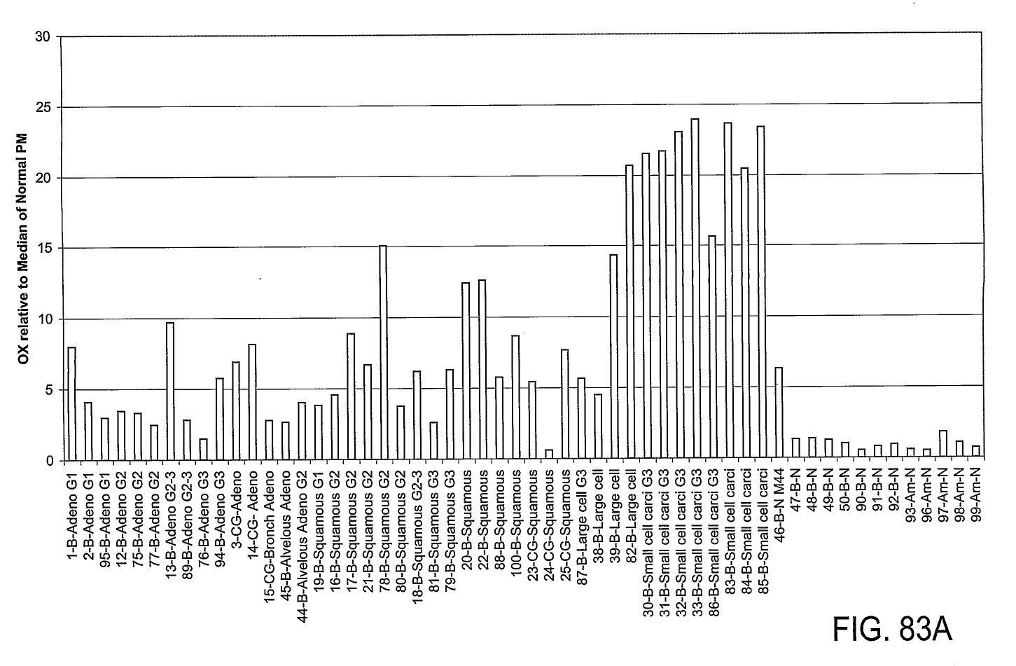

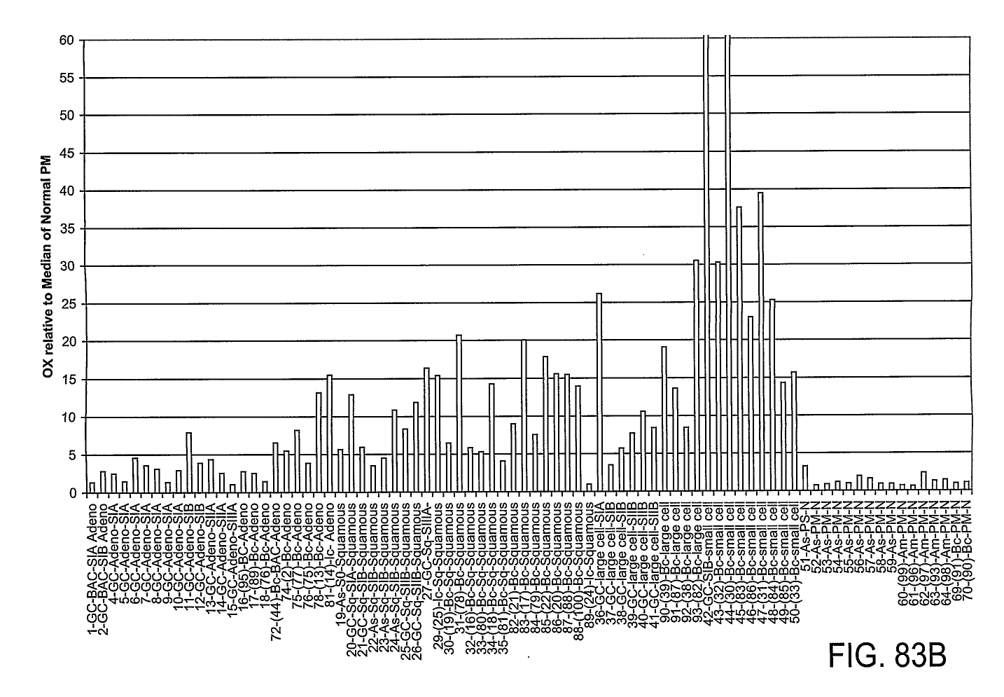

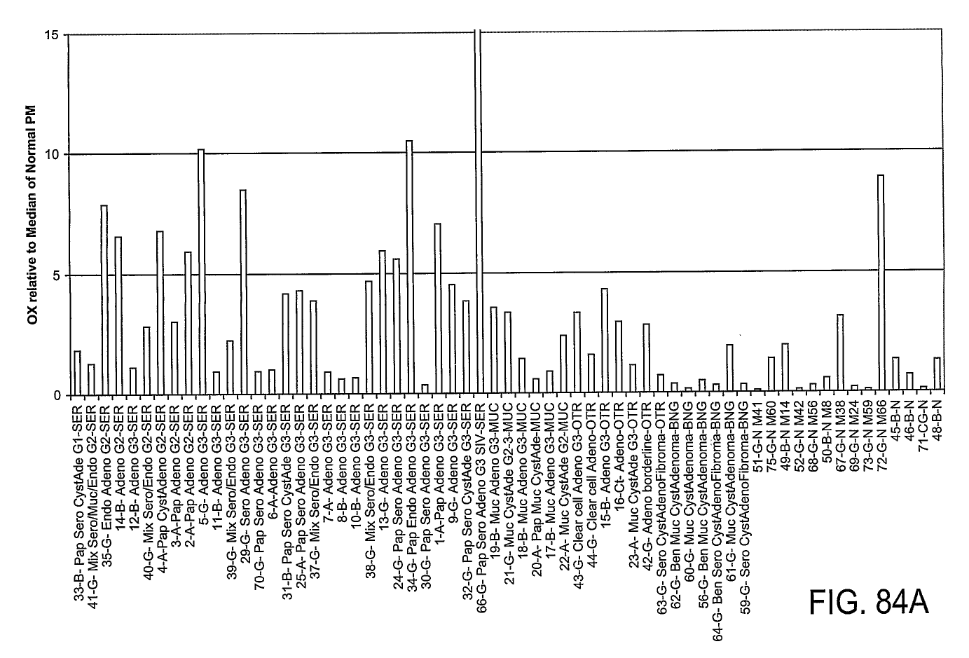

FIG. 83A is a histogram showing over expression of the Homo sapiens trophinin associated protein (tastin) (TROAP) transcripts detectable by or according to T86235_seg46-47WT (SEQ ID NO: 288) in cancerous Lung samples relative to the normal samples.

As is evident from FIG. 83A, the expression of Homo sapiens trophinin associated protein (tastin) (TROAP) transcripts detectable by the above amplicon in non-small cell carcinoma and small cell carcinoma and non-small cell carcinoma samples was significantly higher than in the non-cancerous samples (sample numbers 47, 48, 49, 50, 90, 91, 92, 93, 96, 97, 98 and 99, Table 1_{—}2 above). Notably an over-expression of at least 5 fold was found in 19 out of 35 non-small cell carcinoma samples, specifically in 5 out of 15 adenocarcinoma samples, 11 out of 16 squamous cell carcinoma samples and 3 out of 4 large cell carcinoma samples, and in 8 out of 8 small cell carcinoma samples.

Statistical analysis was applied to verify the significance of these results, as described below.

The P value for the difference in the expression levels of Homo sapiens trophinin associated protein (tastin) (TROAP) transcripts detectable by the above amplicon in all Lung non-small cell carcinoma samples versus the normal tissue samples was determined by T test as 1.36e-08. The P value for the difference in the expression levels of Homo sapiens trophinin associated protein (tastin) (TROAP) transcripts detectable by the above amplicon in Lung adenocarcinoma samples versus the normal tissue samples was determined by T test as 6.93e-05. The P value for the difference in the expression levels of Homo sapiens trophinin associated protein (tastin) (TROAP) transcripts detectable by the above amplicon in Lung squamous cell carcinoma samples versus the normal tissue samples was determined by T test as 1.79e-05. The P value for the difference in the expression levels of Homo sapiens trophinin associated protein (tastin) (TROAP) transcripts detectable by the above amplicon in Lung small cell carcinoma samples versus the normal tissue samples was determined by T test as 9.27e-008.

Threshold of 5 fold over expression was found to differentiate between non-small cell carcinoma and normal samples with P value of 5.82e-04 as checked by exact Fisher test. Threshold of 5 fold over expression was found to differentiate between adenocarcinoma and normal samples with P value of 3.72e-02 as checked by exact Fisher test. Threshold of 5 fold over expression was found to differentiate between squamous cell carcinoma and normal samples with P value of 2.03e-04 as checked by exact Fisher test. Threshold of 5 fold over expression was found to differentiate between large cell carcinoma and normal samples with P value of 7.14e-03 as checked by exact Fisher test. Threshold of 5 fold over expression was found to differentiate between small cell carcinoma and normal samples with P value of 7.94e-06 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T86235_seg46-47WTF (SEQ ID NO: 286) forward primer; and T86235_seg46-47WTR (SEQ ID NO: 287) reverse primer.

The present invention also preferably encompasses any amplicon obtained through the use of any suitable primer pair; for example, for the above experiment, the following amplicon was obtained as a non-limiting illustrative example only of a suitable amplicon: T86235_seg46-47WT (SEQ ID NO: 288).

Expression of Homo sapiens trophinin associated protein (tastin) (TROAP) transcripts detectable by or according to seg46-47WT—T86235_seg46-47WT (SEQ ID NO: 288) amplicon and primers T86235_seg46-47WTF (SEQ ID NO: 286) and T86235_seg46-47WTR (SEQ ID NO: 287) was further measured by real time PCR using enlarged panel as shown in Table 1_{—}6. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 51-64, 69 and 70, Table 1_{—}6 above), to obtain a value of fold up-regulation for each sample relative to the median of the normal samples.

FIG. 83B is a histogram showing over expression of the above-indicated Homo sapiens trophinin associated protein (tastin) (TROAP) transcripts in cancerous Lung samples relative to the normal samples.

As is evident from FIG. 83B, the expression of Homo sapiens trophinin associated protein (tastin) (TROAP) transcripts detectable by the above amplicon in small cell carcinoma and non-small cell carcinoma samples was significantly higher than in the non-cancerous samples (sample numbers 51-64, 69 and 70, Table 1_{—}6 above). Notably an over-expression of at least 5 fold was found in 35 out of 57 non-small cell carcinoma samples, specifically in 6 out of 23 adenocarcinoma samples, in 20 out of 24 squamous cell carcinoma samples, and in 9 out of 10 large cell carcinoma samples, and also in 9 out of 9 small cell carcinoma samples.

Statistical analysis was applied to verify the significance of these results, as described below.

The P value for the difference in the expression levels of Homo sapiens trophinin associated protein (tastin) (TROAP) transcripts detectable by the above amplicon in Lung non-small cell carcinoma samples versus the normal tissue samples was determined by T test as 3.06E-11. The P value for the difference in the expression levels of Homo sapiens trophinin associated protein (tastin) (TROAP) transcripts detectable by the above amplicon in Lung adenocarcinoma samples versus the normal tissue samples was determined by T test as 4.91E-04. The P value for the difference in the expression levels of Homo sapiens trophinin associated protein (tastin) (TROAP) transcripts detectable by the above amplicon in Lung squamous cell carcinoma samples versus the normal tissue samples was determined by T test as 3.72E-08. The P value for the difference in the expression levels of Homo sapiens trophinin associated protein (tastin) (TROAP) transcripts detectable by the above amplicon in Lung large cell carcinoma samples versus the normal tissue samples was determined by T test as 2.20E-03. The P value for the difference in the expression levels of Homo sapiens trophinin associated protein (tastin) (TROAP) transcripts detectable by the above amplicon in Lung small cell carcinoma samples versus the normal tissue samples was determined by T test as 1.1 E-03.

Threshold of 5 fold over expression was found to differentiate between non-small cell carcinoma and normal samples with P value of 4.22E-06 as checked by exact Fisher test. Threshold of 5 fold over expression was found to differentiate between adenocarcinoma and normal samples with P value of 3.09E-02 as checked by exact Fisher test. Threshold of 5 fold over expression was found to differentiate between squamous cell carcinoma and normal samples with P value of 7.71E-08 as checked by exact Fisher test. Threshold of 5 fold over expression was found to differentiate between large cell carcinoma and normal samples with P value of 3.20E-06 as checked by exact Fisher test. Threshold of 5 fold over expression was found to differentiate between small cell carcinoma and normal samples with P value of 4.89E-07 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T86235_seg46-47WTF (SEQ ID NO: 286) forward primer; and T86235_seg46-47WTR (SEQ ID NO: 287) reverse primer.

The present invention also preferably encompasses any amplicon obtained through the use of any suitable primer pair; for example, for the above experiment, the following amplicon was obtained as a non-limiting illustrative example only of a suitable amplicon: T86235_seg46-47WT (SEQ ID NO: 288).

Expression of Homo sapiens Trophinin Associated Protein (tastin) (TROAP) T86235 Transcripts which are Detectable by Amplicon as Depicted in Sequence Name T86235_seg46-47WT (SEQ ID NO: 288) in Normal and Cancerous Ovary Tissues

Expression of Homo sapiens trophinin associated protein (tastin) (TROAP) transcripts detectable by or according to seg46-47WT—T86235_seg46-47WT (SEQ ID NO: 288) amplicon and primers T86235_seg46-47WTF (SEQ ID NO: 286) and T86235_seg46-47WTR (SEQ ID NO: 287) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)) and GAPDH (GenBank Accession No. BC026907 (SEQ ID NO:338); GAPDH amplicon (SEQ ID NO: 308)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 45, 46, 71 and 48, Table 1_{—}1 above), to obtain a value of fold up-regulation for each sample relative to the median of the normal PM samples.

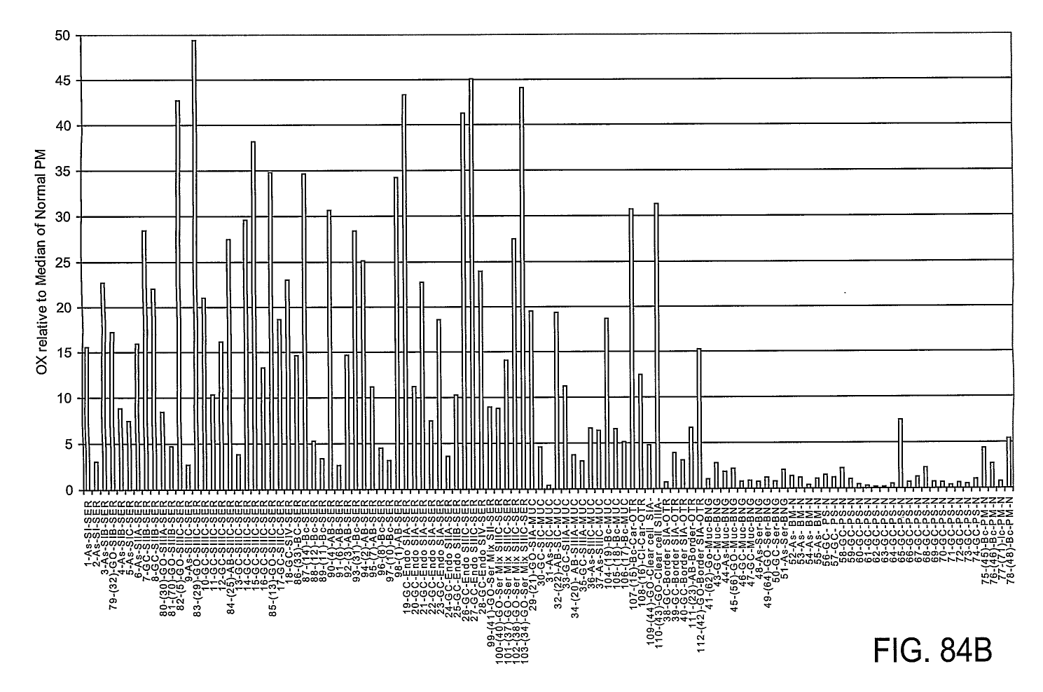

FIG. 84A is a histogram showing over expression of the Homo sapiens trophinin associated protein (tastin) (TROAP) transcripts detectable by or according to T86235_seg46-47WT (SEQ ID NO: 288) in cancerous Ovary samples relative to the normal samples.

As is evident from FIG. 84A, the expression of Homo sapiens trophinin associated protein (tastin) (TROAP) transcripts detectable by the above amplicon in serous carcinoma and adenocarcinoma samples was higher than in the non-cancerous samples (sample numbers 45, 46, 71 and 48, Table 1_{—}1 above). Notably an over-expression of at least 5 fold was found in 11 out of 30 serous carcinoma samples.

Statistical analysis was applied to verify the significance of these results, as described below.

The P value for the difference in the expression levels of Homo sapiens trophinin associated protein (tastin) (TROAP) transcripts detectable by the above amplicon in Ovary serous carcinoma samples versus the normal tissue samples was determined by T test as 2.42e-05.

The above values demonstrate statistical significance of the results.

Primer pairs are also optionally and preferably encompassed within the present invention; for example, for the above experiment, the following primer pair was used as a non-limiting illustrative example only of a suitable primer pair: T86235_seg46-47WTF (SEQ ID NO: 286) forward primer; and T86235_seg46-47WTR (SEQ ID NO: 287) reverse primer.

The present invention also preferably encompasses any amplicon obtained through the use of any suitable primer pair; for example, for the above experiment, the following amplicon was obtained as a non-limiting illustrative example only of a suitable amplicon: T86235_seg46-47WT (SEQ ID NO: 288).

Expression of sapiens trophinin associated protein (tastin) (TROAP) transcripts detectable by or according to seg46-47WT—T86235_seg46-47WT (SEQ ID NO: 288) amplicon and primers T86235_seg46-47WTF (SEQ ID NO: 286) and T86235_seg46-47WTR (SEQ ID NO: 287) was further measured by real time PCR using enlarged panel as shown in Table 1_{—}5. In parallel the expression of three housekeeping genes—SDHA (GenBank Accession No. NM_{—}004168 (SEQ ID NO:335); amplicon—SDHA-amplicon (SEQ ID NO:299)), HPRT1 (GenBank Accession No. NM_{—}000194 (SEQ ID NO:337); amplicon—HPRT1-amplicon (SEQ ID NO: 305) (SEQ ID NO: 305)), G6PD (GenBank Accession No. NM_{—}000402 (SEQ ID NO:339); G6PD amplicon (SEQ ID NO: 311)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 52, 53, 54, 55, 56, 57, 58, 59, 60, 61, 62, 63, 64, 65, 66, 67, 68, 69, 70, 71, 72, 73, 74, 75, 76, 77 and 78, Table 1_{—}5 above), to obtain a value of fold up-regulation for each sample relative to the median of the normal samples.

FIG. 84B is a histogram showing over expression of the above-indicated sapiens trophinin associated protein (tastin) (TROAP) transcripts in cancerous Ovary samples relative to the normal samples.

As is evident from FIG. 84B, the expression of sapiens trophinin associated protein (tastin) (TROAP) transcripts detectable by the above amplicon in adenocarcinoma samples was significantly higher than in the non-cancerous samples (sample numbers 41, 43-78, Table 1_{—}5 above). Notably an over-expression of at least 5 fold was found in 57 out of 74 adenocarcinoma samples, specifically in 30 out of 38 serous carcinoma samples, in 9 out of 10 endometroid type adenocarcinoma, in 8 out of 12 mucinous carcinoma samples.

Statistical analysis was applied to verify the significance of these results, as described below.

The P value for the difference in the expression levels of sapiens trophinin associated protein (tastin) (TROAP) transcripts detectable by the above amplicon in Ovary adenocarcinoma samples versus the non-cancerous tissue samples was determined by T test as 4.44e-16. The P value for the difference in the expression levels of sapiens trophinin associated protein (tastin) (TROAP) transcripts detectable by the above amplicon in Ovary serous carcinoma samples versus the non-cancerous tissue samples was determined by T test as 2.88e-10. The P value for the difference in the expression levels of sapiens trophinin associated protein (tastin) (TROAP) transcripts detectable by the above amplicon in Ovary mucinous carcinoma samples versus the non-cancerous tissue samples was determined by T test as 9 3.48e-03.

Threshold of 5 fold over expression was found to differentiate between adenocarcinoma and non-cancerous samples with P value of 9.86e-14 as checked by exact Fisher test. Threshold of 5 fold over expression was found to differentiate between serous carcinoma and non-cancerous samples with P value of 2.12e-11 as checked by exact Fisher test. Threshold of 5 fold over expression was found to differentiate between mucinous carcinoma and non-cancerous samples with P value of 4.11e-05 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T86235_seg46-47WTF (SEQ ID NO: 286) forward primer; and T86235_seg46-47WTR (SEQ ID NO: 287) reverse primer.

The present invention also preferably encompasses any amplicon obtained through the use of any suitable primer pair; for example, for the above experiment, the following amplicon was obtained as a non-limiting illustrative example only of a suitable amplicon: T86235_seg46-47WT (SEQ ID NO: 288).

Expression of Homo sapiens Trophinin Associated Protein (tastin) (TROAP) T86235 Transcripts which are Detectable by Amplicon as Depicted in Sequence Name T86235_seg46-47WT (SEQ ID NO: 288) in Different Normal Tissues

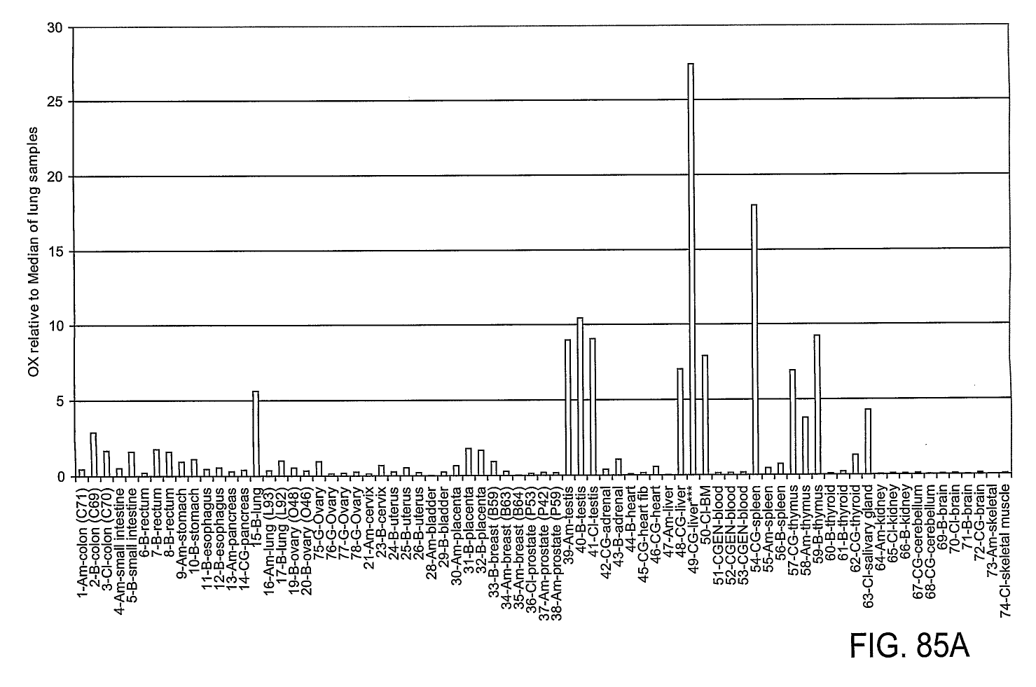

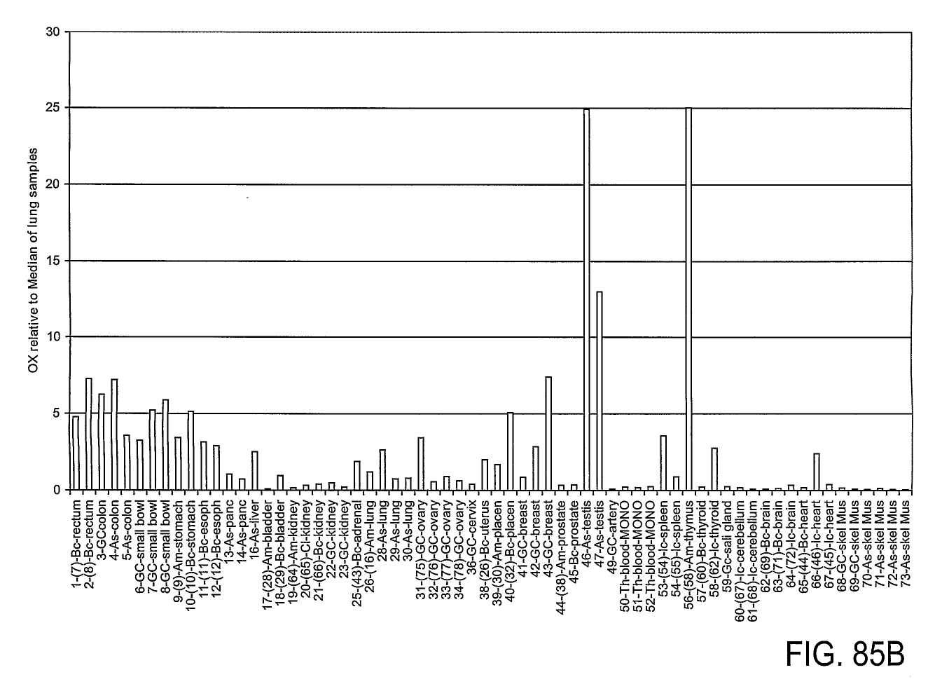

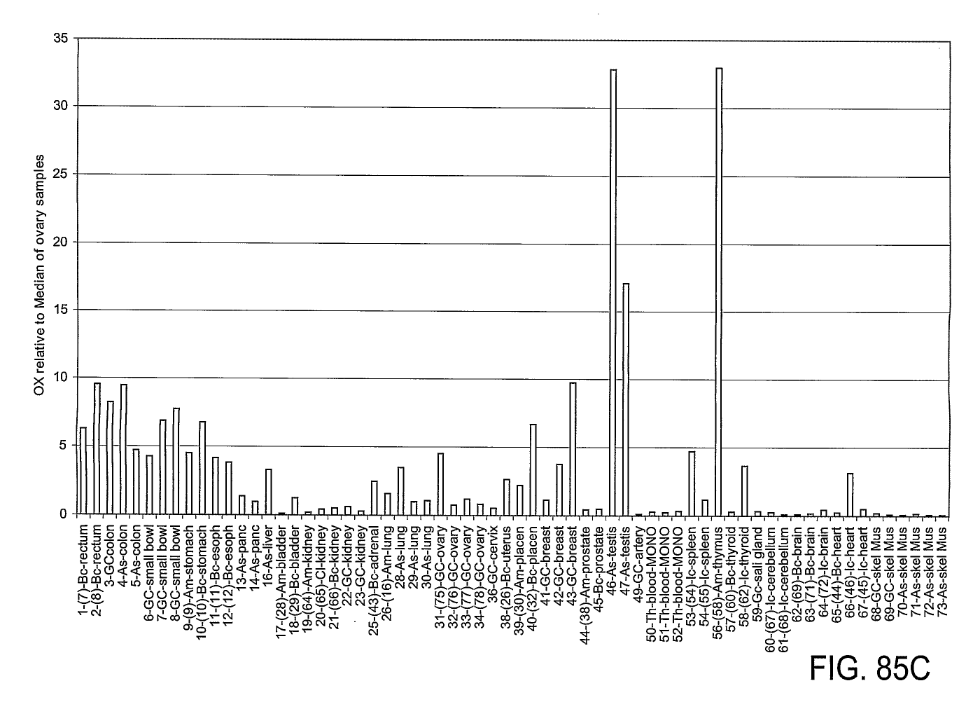

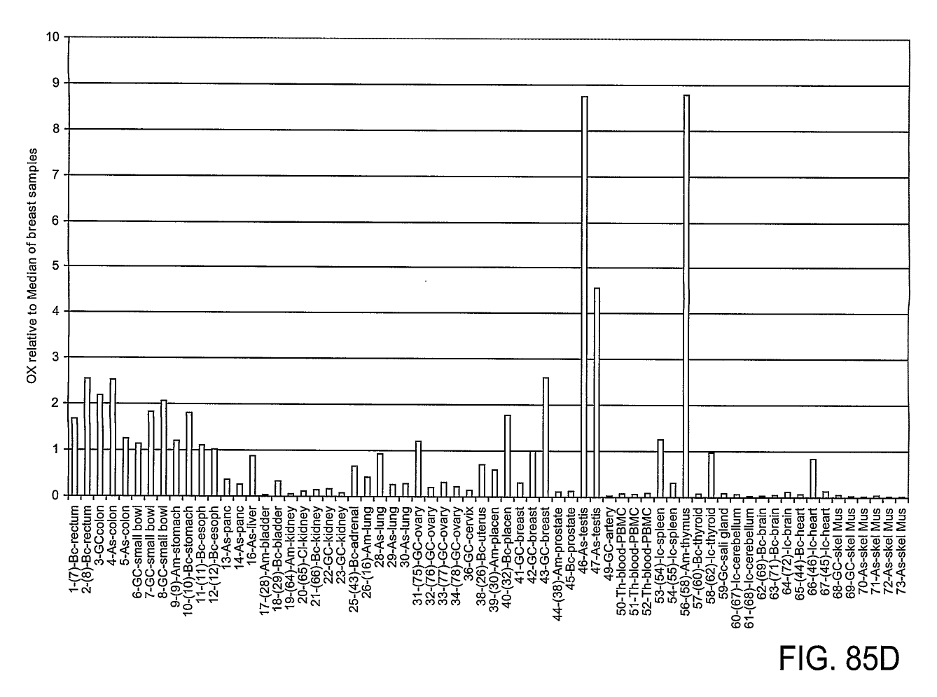

Expression of Homo sapiens trophinin associated protein (tastin) (TROAP) transcripts detectable by or according to seg46-47WT—T86235_seg46-47WT (SEQ ID NO: 288) amplicon and primers T86235_seg46-47WTF (SEQ ID NO: 286) and T86235 seg46-47WTR (SEQ ID NO: 287) was measured by real time PCR. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), RPL19 (GenBank Accession No. NM_{—}000981 (SEQ ID NO:342); RPL19 amplicon (SEQ ID NO: 320)) and TATA box (GenBank Accession No. NM_{—}003194 (SEQ ID NO:343); TATA amplicon (SEQ ID NO: 323)) was measured similarly. For each RT sample, the expression of the above amplicon was normalized to the geometric mean of the quantities of the housekeeping genes. The normalized quantity of each RT sample was then divided by the median of the quantities of the lung samples (sample numbers 15, 16 and 17, Table 1_{—}9 above), to obtain a value of relative expression of each sample relative to the median of the lung samples. FIG. 85A shows expression of Homo sapiens trophinin associated protein (tastin) (TROAP) T86235 transcripts which are detectable by amplicon as depicted in sequence name T86235_seg46-47WT (SEQ ID NO: 288) in different normal tissues.

Expression of Homo sapiens trophinin associated protein (tastin) (TROAP) transcripts detectable by or according to seg46-47WT—T86235_seg46-47WT (SEQ ID NO: 288) amplicon and primers T86235_seg46-47WTF (SEQ ID NO: 286) and T86235_seg46-47WTR (SEQ ID NO: 287) was further measured by real time PCR using updated panel as shown in Table 1_{—}10. In parallel the expression of four housekeeping genes—SDHA (GenBank Accession No. NM_{—}004168 (SEQ ID NO:335); amplicon—SDHA-amplicon (SEQ ID NO:299)), Ubiquitin (GenBank Accession No. BC000449 (SEQ ID NO:341); amplicon—Ubiquitin-amplicon (SEQ ID NO: 317)), and TATA box (GenBank Accession No. NM_{—}003194 (SEQ ID NO:343); TATA amplicon (SEQ ID NO: 323)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of:

A. The Lung samples (sample numbers 26, 28, 29 and 30, Table 1_{—}10 above), to obtain a value of relative expression of each sample relative to the median of the Lung samples—as presented in FIG. 85B.
B. The ovary samples (sample numbers 31-34, Table 1_{—}10 above), to obtain a value of relative expression of each sample relative to the median of the Ovary samples—as presented in FIG. 85C.
C. The breast samples (sample numbers 41-43, Table 1_{—}10 above), to obtain a value of relative expression of each sample relative to the median of the Breast samples—as presented in FIG. 85D.
Expression of Homo sapiens Trophinin Associated Protein (tastin) (TROAP) T86235 Transcripts which are Detectable by Amplicon as Depicted in Sequence Name T86235_seg44 (SEQ ID NO: 285) in Normal and Cancerous Lung Tissues

Expression of Homo sapiens trophinin associated protein (tastin) (TROAP) transcripts detectable by or according to seg44—T86235_seg44 (SEQ ID NO: 285) amplicon and primers T86235_seg44F (SEQ ID NO: 283) and T86235_seg44R (SEQ ID NO: 284) was measured by real time PCR. In parallel the expression of four housekeeping genes—HPRT1 (GenBank Accession No. NM_{—}000194 (SEQ ID NO:337); amplicon—HPRT1-amplicon (SEQ ID NO: 305) (SEQ ID NO: 305)), PBGD (GenBank Accession No. BC019323 (SEQ ID NO:336); amplicon—PBGD-amplicon (SEQ ID NO: 302)), SDHA (GenBank Accession No. NM_{—}004168 (SEQ ID NO:335); amplicon—SDHA-amplicon (SEQ ID NO:299)) and Ubiquitin (GenBank Accession No. BC000449 (SEQ ID NO:341); amplicon—Ubiquitin-amplicon (SEQ ID NO: 317)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 51-64, 69 and 70, Table 1_{—}6 above), to obtain a value of fold up-regulation for each sample relative to the median of the normal samples.

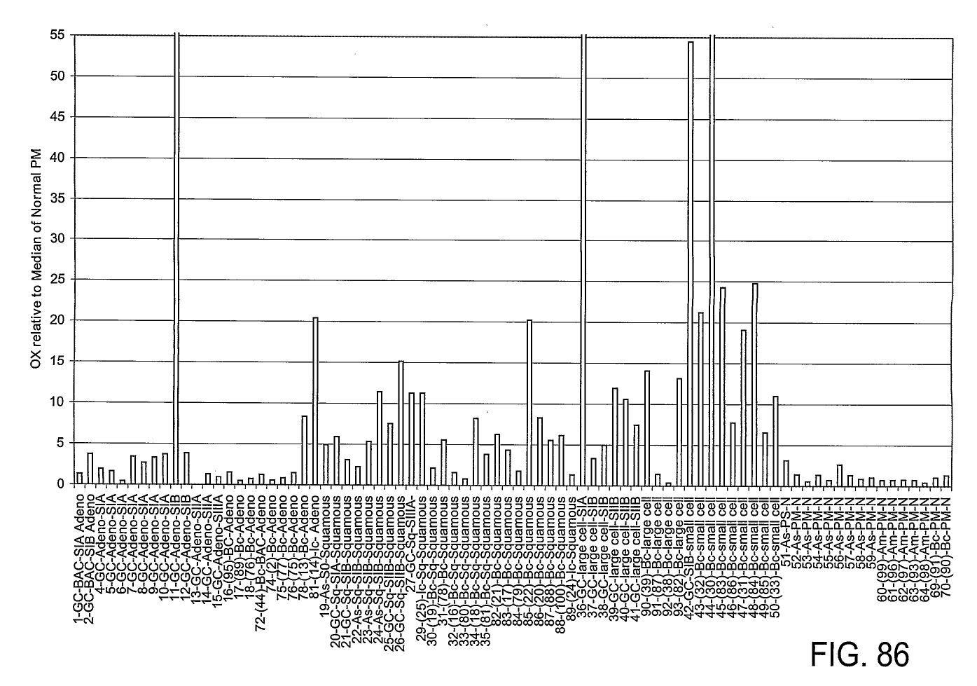

FIG. 86 is a histogram showing over expression of the above-indicated Homo sapiens trophinin associated protein (tastin) (TROAP) transcripts in cancerous Lung samples relative to the normal samples.

As is evident from FIG. 86, the expression of Homo sapiens trophinin associated protein (tastin) (TROAP) transcripts detectable by the above amplicon in small cell carcinoma and non-small cell carcinoma samples was significantly higher than in the non-cancerous samples (sample numbers 51-64, 69 and 70, Table 1_{—}6 above). Notably an over-expression of at least 5 fold was found in 23 out of 57 non-small cell carcinoma samples, specifically in 3 out of 23 adenocarcinoma samples, in 14 out of 24 squamous cell carcinoma samples and in 6 out of 10 large cell carcinoma samples, and also in 9 out of 9 small cell carcinoma samples.

Statistical analysis was applied to verify the significance of these results, as described below.

The P value for the difference in the expression levels of Homo sapiens trophinin associated protein (tastin) (TROAP) transcripts detectable by the above amplicon in Lung squamous cell carcinoma samples versus the normal tissue samples was determined by T test as 1.64E-05. The P value for the difference in the expression levels of Homo sapiens trophinin associated protein (tastin) (TROAP) transcripts detectable by the above amplicon in Lung small cell carcinoma samples versus the normal tissue samples was determined by T test as 5.39E-03.

Threshold of 5 fold over expression was found to differentiate between non-small cell carcinoma and normal samples with P value of 9.34E-04 as checked by exact Fisher test. Threshold of 5 fold over expression was found to differentiate between squamous cell carcinoma and normal samples with P value of 8.45E-05 as checked by exact Fisher test. Threshold of 5 fold over expression was found to differentiate between large cell carcinoma and normal samples with P value of 9.12E-04 as checked by exact Fisher test. Threshold of 5 fold over expression was found to differentiate between small cell carcinoma and normal samples with P value of 4.89E-07 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T86235_seg44F (SEQ ID NO: 283) forward primer; and T86235_seg44R (SEQ ID NO: 284) reverse primer.

The present invention also preferably encompasses any amplicon obtained through the use of any suitable primer pair; for example, for the above experiment, the following amplicon was obtained as a non-limiting illustrative example only of a suitable amplicon: T86235_seg44 (SEQ ID NO: 285).

Expression of Homo sapiens Trophinin Associated Protein (tastin) (TROAP) T86235 Transcripts which are Detectable by Amplicon as Depicted in Sequence Name T86235_seg44 (SEQ ID NO: 285) in Normal and Cancerous Colon Tissues

Expression of Homo sapiens trophinin associated protein (tastin) (TROAP) transcripts detectable by or according to seg44, T86235_seg44 (SEQ ID NO: 285) amplicon and primers T86235_seg44F (SEQ ID NO: 283) T86235_seg44R (SEQ ID NO: 284) was measured by real time PCR. In parallel the expression of three housekeeping genes—PBGD (GenBank Accession No. BC019323 (SEQ ID NO:336); amplicon—PBGD-amplicon (SEQ ID NO: 302)), HPRT1 (GenBank Accession No. NM_{—}000194 (SEQ ID NO:337); amplicon—HPRT1-amplicon (SEQ ID NO: 305) (SEQ ID NO: 305)) and G6PD (GenBank Accession No. NM_{—}000402 (SEQ ID NO:339); G6PD amplicon (SEQ ID NO: 311)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (Sample Nos. 52, 42, 43, 44, 45, 46, 47, 48, 49, 50, 51, 53, 54, 55, 56, 57, 58, 59, 60, 61, 62, 63 and 64, Table 1_{—}8, above), to obtain a value of fold differential expression for each sample relative to the median of the normal samples.

In one experiment that was carried out no differential expression in the cancerous samples relative to the normal samples was observed.

Primer pairs are also optionally and preferably encompassed within the present invention; for example, for the above experiment, the following primer pair was used as a non-limiting illustrative example only of a suitable primer pair: T86235_seg44F (SEQ ID NO: 283) forward primer; and T86235_seg44R (SEQ ID NO: 284) reverse primer.

The present invention also preferably encompasses any amplicon obtained through the use of any suitable primer pair; for example, for the above experiment, the following amplicon was obtained as a non-limiting illustrative example only of a suitable amplicon: T86235_seg44 (SEQ ID NO: 285).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-62. (canceled)

63. An isolated polynucleotide comprising a nucleic acid sequence set forth in a member selected from the group consisting of SEQ ID NOs: 1-3, 334 and homologues and fragments thereof.

64. The polynucleotide of claim 63, wherein the nucleic acid sequence encodes a polypeptide having an amino acid sequence set forth in a member selected from the group consisting of SEQ ID NOs: 33-36.

65. The polynucleotide of claim 63, wherein the nucleic acid sequence encodes a polypeptide comprising contiguous amino acids having at least about 70%, 80%, 85%, 90%, 95% or 100% homology to SEQ ID NO:494

66. The polynucleotide of claim 63, wherein the nucleic acid sequence encodes a polypeptide comprising contiguous amino acids having at least about 70%, 80%, 85%, 90%, 95% or 100% homology to SEQ ID NO: 495

67. The polynucleotide of claim 63, wherein the nucleic acid sequence encodes a polypeptide comprising contiguous amino acids having at least about 70%, 80%, 85%, 90%, 95% or 100% homology to SEQ ID NO: 496.

68. The polynucleotide of claim 63, wherein the nucleic acid sequence encodes a polypeptide comprising contiguous amino acids having at least about 70%, 80%, 85%, 90%, 95% or 100% homology to SEQ ID NO: 497.

69. The polynucleotide of claim 63, wherein the nucleic acid sequence encodes a polypeptide comprising contiguous amino acids having at least about 70%, 80%, 85%, 90%, 95% or 100% homology to SEQ ID NO: 498.

70. An isolated polynucleotide, comprising a nucleic acid sequence complementary to any one of the nucleic acid sequences of claim 63.

71. An isolated polynucleotide, comprising a nucleic acid sequence that hybridizes under stringent conditions to any one of the nucleic acid sequences of claim 63.

72. An isolated polypeptide having an amino acid sequence encoded by the nucleic acid sequence of claim 63.

73. An isolated polypeptide having an amino acid sequence as set forth in any one of SEQ ID NOs: 33-36.

74. The polypeptide of claim 72, comprising a first portion having an amino acid sequence being at least about 90% homologous to amino acids 1-17 of SEQ ID NO: 33, a second portion having amino acid sequence being at least about 70%, 80%, 85%, 90%, 95% or 100% homologous to SEQ ID NO: 494, and a third portion having amino acid sequence being at least about 90% homologous to amino acids 57-290 of SEQ ID NO: 33, wherein the first amino acid sequence, the second amino acid sequence and the third amino acid sequence are contiguous and in a sequential order.

75. The polypeptide of claim 72, comprising a first portion having an amino acid sequence being at least about 70%, 80%, 85%, 90%, 95% or 100% homologous to SEQ ID NO: 495, and a second portion having an amino acid sequence being at least about 90% homologous to amino acids 18-457 of SEQ ID NO: 34, wherein the first amino acid sequence and the second amino acid sequence are contiguous and in a sequential order.

76. The polypeptide of claim 72, comprising a first portion having amino acid sequence being at least about 70%, 80%, 85%, 90%, 95% or 100% homologous to SEQ ID NO: 496, a second portion having amino acid sequence being at least about 90% homologous to amino acids 168-184 of SEQ ID NO: 34, a third portion having amino acid sequence being at least about at least about 70%, 80%, 85%, 90%, 95% or 100% homologous to SEQ ID NO: 494, and a fourth portion having amino acid sequence being at least about 90% homologous to acids 224-457 of SEQ ID NO: 34, wherein the first amino acid sequence, the second amino acid sequence, the third amino acid sequence and the fourth amino acid sequence are contiguous and in a sequential order.

77. The polypeptide of claim 72, comprising a first portion having an amino acid sequence being at least about 70%, 80%, 85%, 90%, 95% or 100% homologous to SEQ ID NO: 495, a second portion having amino acid sequence being at least about 90% homologous to amino acids 18-349 of SEQ ID NO: 36, and a third portion having amino acid sequence LG corresponding to amino acids 350-351 of SEQ ID NO: 36, wherein the first amino acid sequence, the second amino acid sequence and the third amino acid sequence are contiguous and in a sequential order.

78. A polypeptide comprising an amino acid sequence at least about 70%, 80%, 85%, 90%, 95% or 100% homologous to SEQ ID NO:494.

79. A polypeptide comprising an amino acid sequence at least about 70%, 80%, 85%, 90%, 95% or 100% homologous to SEQ ID NO:495.

80. A polypeptide comprising an amino acid sequence at least about 70%, 80%, 85%, 90%, 95% or 100% homologous to SEQ ID NO:496.

81. A polypeptide comprising an amino acid sequence at least about 70%, 80%, 85%, 90%, 95% or 100% homologous to SEQ ID NO:497.

82. A polypeptide comprising an amino acid sequence at least about 70%, 80%, 85%, 90%, 95% or 100% homologous to SEQ ID NO:498.

83. An antibody which binds to at least one epitope of a polypeptide having an amino acid sequence according to claim 72.

84. The antibody of claim 83, wherein said antibody is capable of differentiating between a splice variant having said epitope and a corresponding known protein.

85. An expression vector comprising the polynucleotide sequence according to claim 63.

86. A host cell comprising the vector according to claim 85.

87. A process for producing a polypeptide comprising: culturing the host cell according to claim 86 under conditions suitable to produce the polypeptide encoded by said polynucleotide; and recovering said polypeptide.

88. An isolated polynucleotide segment, comprising a nucleic acid sequence selected from the group consisting of: SEQ ID NOs: 4-28.

89. An isolated polynucleotide segment comprising a nucleic acid sequence complementary to any one of the nucleic acid sequences of the segments of claim 88.

90. An isolated polynucleotide segment comprising a nucleic acid sequence that hybridizes under stringent conditions to any one of the nucleic acid sequences of the segments of claim 88.

91. A kit for detecting cancer, comprising a marker capable of detecting at least one polypeptide according to claim 72 or a part thereof.

92. The kit of claim 91, wherein said kit comprises an antibody according to claim 18 and wherein said kit further comprises at least one reagent for performing an immunoassay.

93. The kit of claim 92, wherein said immunoassay is selected from the group consisting of an ELISA, a RIA (radio immunoassay), a slot blot, immunohistochemical assay, FACS (fluorescence activated cell sorting), a radio-imaging assay or a Western blot.

94. The kit of claim 91, wherein the cancer is breast cancer.

95. The kit of claim 91, wherein the cancer is invasive or metastatic.

96. The kit of claim 94, wherein the breast cancer comprises cancers of the breast or surrounding tissue, or an indicative condition.

97. The kit of claim 96, wherein the breast cancer is selected from the group consisting of ductal carcinoma (in-situ or invasive), lobular carcinoma (in situ or invasive), inflammatory breast cancer, mucinous carcinoma, tubular carcinoma, or Paget's disease of the nipple.

98. The kit of claim 96, wherein the indicative condition is selected from the group consisting of ductal hyperplasia without atypia and atypical hyperplasia.

99. A kit for detecting cancer, comprising a marker capable of detecting at least one polynucleotide according to claim 63.

100. The kit of claim 99, wherein said kit comprises at least one nucleotide probe or primer pair.

101. The kit of claim 100, wherein said kit comprises at least one oligonucleotide capable of selectively hybridizing to a nucleic acid sequence as set forth in any one of SEQ ID NOs:1-28, 334, 358, 361, or a homologue or fragment thereof.

102. The kit of claim 100, wherein said at least one primer pair amplifies an amplicon comprising the sequence as set forth in SEQ ID NO:358 or 361.

103. The primer pair of claim 102, comprising a pair of isolated oligonucleotides selected from the group consisting of SEQ ID NO:356 and SEQ ID NO:357; and SEQ ID NO:359 and SEQ ID NO:360.

104. The kit of claim 99, wherein said kit comprises at least one oligonucleotide capable of selectively hybridizing to a polynucleotide according to claim 1.

105. A method for detecting cancer and/or monitoring disease progression and/or monitoring treatment efficacy and/or detecting acute or chronic exacerbation of cancer and/or selecting a therapy for cancer comprising detecting in a sample differential expression of at least one polypeptide according to claim 72 or a part thereof.

106. The method of claim 105, wherein the cancer is breast cancer.

107. The method of claim 106, wherein said cancer is invasive or metastatic.

108. The method of claim 106, wherein the breast cancer comprises cancers of the breast or surrounding tissue, or an indicative condition.

109. The method of claim 108, wherein the breast cancer is selected from the group consisting of ductal carcinoma (in-situ or invasive), lobular carcinoma (in situ or invasive), inflammatory breast cancer, mucinous carcinoma, tubular carcinoma, or Paget's disease of the nipple.

110. The method of claim 108, wherein the indicative condition is selected from the group consisting of ductal hyperplasia without atypia and atypical hyperplasia.

111. The method of claim 105, wherein detecting the differential expression of the polypeptide is performed with an antibody which binds to at least one epitope of a polypeptide of at least 70%, 80%, 85%, 95%, or 100% homologous to SEQ ID NOS:494-498.

112. A method for detecting cancer and/or monitoring disease progression and/or monitoring treatment efficacy and/or detecting acute or chronic exacerbation of cancer and/or selecting a therapy for cancer comprising detecting in a sample differential expression of at least one polynucleotide according to claim 63 or a part thereof.

113. The method of claim 112, wherein the cancer is breast cancer.

114. The method of claim 113, wherein said cancer is invasive or metastatic.

115. The method of claim 113, wherein the breast cancer comprises cancers of the breast or surrounding tissue, or an indicative condition.

116. The method of claim 115, wherein the breast cancer is selected from the group consisting of ductal carcinoma (in-situ or invasive), lobular carcinoma (in situ or invasive), inflammatory breast cancer, mucinous carcinoma, tubular carcinoma, or Paget's disease of the nipple.

117. The method of claim 115, wherein the indicative condition is selected from the group consisting of ductal hyperplasia without atypia and atypical hyperplasia.
